(19) United States
(12) Patent Application Publication
(10) Pub. No.: US 2006/0252807 A1
(43) Pub. Date: Nov. 9, 2006

(54) Novel ortho-terphenyl inhibitors of p38 kinase and methods of treating inflammatory disorders

(75) Inventors: Daniel L. Severance, San Diego, CA (US); Elisabeth M. M. Gardiner, San Diego, CA (US); Stewart A. Noble, San Diego, CA (US); Boliang Lou, Louisville, KY (US); Allen J. Borchardt, San Diego, CA (US); Jeffrey R. Roppe, Temecula, CA (US); Mehmet Kahraman, San Diego, CA (US); Dana L. Siegel, San Diego, CA (US); Shawn A. Scranton, San Diego, CA (US)

Correspondence Address: INTERNATIONAL PATENT GROUP;ATTN: SAL GRILLO, P.O. BOX 38129, ST. LOUIS, MO, 63138, US

(73) Assignee: Kalypsys, Inc.

(21) Appl. No.: 11/409,451

(22) Filed: Apr. 20, 2006

Related U.S. Application Data
(60) Provisional application No. 60/674,047, filed on Apr. 22, 2005.
(60) Provisional application No. 60/776,594, filed on Feb. 24, 2006.

(51) Int. Cl. C07D 417/04; A61K 31/433; A61K 31/426

(52) U.S. Cl. 514/362; 548/137; 514/365; 548/190

(57) ABSTRACT

The present invention relates to compounds and methods useful as inhibitors of p38 kinase for the treatment or prevention and treatment of diseases such as inflammatory diseases, autoimmune diseases, destructive bone disorders, proliferative disorders, angiogenic disorders, infectious diseases, neurodegenerative diseases, and viral diseases.

This application claims the priority of U.S. provisional application 60/674,047, filed Apr. 22, 2005 and U.S. provisional application 60/776,594, filed Feb. 24, 2006.

### FIELD OF THE INVENTION

The present invention is directed to new ortho-terphenyl compounds and compositions and their application as pharmaceuticals for the treatment of disease. Methods of inhibition of p38 kinase activity in a human or animal subject are also provided for the treatment diseases such as inflammatory diseases, autoimmune diseases, destructive bone disorders, proliferative disorders, angiogenic disorders, infectious diseases, neurodegenerative diseases, and viral diseases.

### BACKGROUND OF THE INVENTION

The present invention relates to inhibitors of p38, a mammalian protein kinase involved in cell proliferation, cell death and response to extracellular stimuli. The invention also relates to methods for producing these inhibitors. The invention also provides pharmaceutical compositions comprising the inhibitors of the present invention and methods of utilizing those compositions in the treatment and prevention of various disorders. The compounds are potent inhibitors of p38 kinase and are useful in the prophylaxis or treatment of p38 kinase mediated diseases or disorders, such as inflammatory diseases, autoimmune diseases, destructive bone disorders, proliferative disorders, angiogenic disorders, infectious diseases, neurodegenerative diseases, and viral diseases.

Four isoforms of p38 have been described (p38α/β/γ/δ). The human p38α enzyme was initially identified as a target of cytokine-suppressive anti-inflammatory drugs (CSAIDs) and the two isoenzymes found were initially termed CSAID binding protein-1 (CSBP-1) and CSBP-2 [Lee, J. C. et al, Nature (London) 1994, 372, 739-46]. CSBP-2 is now widely referred to as p38α and differs from CSBP-1 in an internal sequence of 25 amino acids as a result of differential splicing of two exons that are conserved in both mouse and human [McDonnell, P. C. et al, Genomics 1995, 29, 301-2]. CSBP-1 and p38α are expressed ubiquitously and there is no difference between the two isoforms with respect to tissue distribution, activation profile, substrate preference or CSAID binding. A second isoform is p38β which has 70% identity with p38α. A second form of p380, termed p38β2, is also known, and of the two this is believed to be the major form. P38α and p38β2 are expressed in many different tissues. However in monocytes and macrophages p38α is the predominant kinase activity [Lee, J. C., ibid; Jing, Y. et al, J. Biol. Chem. 1996, 271, 10531-34; Hale, K. K. et al, J. Immun. 1999, 162, 4246-52]. P38γ and p38δ (also termed SAP kinase-3 and SAP kinase-4 respectively) have .about.63% and .about.61% homology to p38α respectively. P38δ is predominantly expressed in skeletal muscle whilst p38δ is found in testes, pancreas, prostate, small intestine and in certain endocrine tissues.

All p38 homologues and splice variants contain a 12 amino acid activation loop that includes a Thr-Gly-Tyr motif. Dual phosphorylation of both Thr-1180 and Tyr-1182 in the TGY motif by a dual specificity upstream kinase is essential for the activation of p38 and results in a >11000-fold increase in specific activity of these enzymes [Doza, Y. N. et al FEBS Lett., 1995, 364, 7095-8012]. This dual phosphorylation is effected by MKK6 and under certain conditions the related enzyme MKK3 (see FIG. 1) [Enslen, H. et al J. Biol. Chem., 1998, 273, 1741-48]. MKK3 and MKK6 belong to a family of enzymes termed MAPKK (mitogen activating protein kinase kinase) which are in turn activated by MAPKKK (mitogen activating protein kinase kinase kinase) otherwise known as MAP3K.

Several MAP3Ks have been identified that are activated by a wide variety of stimuli including environmental stress, inflammatory cytokines and other factors. MEKK4/MTK1 (MAP or ERK kinase kinase/MAP three kinase-1), ASK1 (apoptosis stimulated kinase) and TAK1 (TGF-β-activated kinase) are some of the enzymes identified as upstream activators of for MAPKKs. MEKK4/MTK1 is thought to be activated by several GADD-45-like genes that are induced in response to environmental stimuli and which eventually lead to p38 activation [Takekawa, M. and Saito, H. Cell, 1998, 95, 521-30]. TAK1 has been shown to activate MKK6 in response to transforming growth factor-β (TGF-β). TNF-stimulated activation of p38 is believed to be mediated by the recruitment of TRAF2 [TNF receptor associated factor] and the Fas adaptor protein, Daxx, which results in the activation of ASK1 and subsequently p38.

Several substrates of p38 have been identified including other kinases [e.g. MAPK activated protein kinase 2/3/5 (MAPKAP 2/3/5), p38 regulated/activated protein kinase (PRAK), MAP kinase-interacting kinase 1/2 (MNK1/2), mitogen- and stress-activated protein kinase 1 (MSK1/RLPK) and ribosomal S6 kinase-B (RSK-B)], transcription factors [e.g. activating transcription factor 2/6 (ATF2/6), monocyte-enhancer factor-2A/C (MEF2A/C), C/EBP homologous protein (CHOP), Elk1 and Sap-1a1] and others substrates [e.g. cPLA2, p47phox].

MAPKAP K2 is activated by p38 in response to environmental stress. Mice engineered to lack MAPKAP K2 do not produce TNF in response to lipopolysaccharide (LPS). Production of several other cytokines such as IL-1, IL-6, IFN-g and IL-10 is also partially inhibited [Kotlyarov, A. et al Nature Cell Biol. 1999, 1, 94-7]. Further, MAPKAP K2 from embryonic stem cells from p38α null mice was not activated in response to stress and these cells did not produce IL-6 in response to IL-1 [Allen, M. et al, J. Exp. Med. 2000, 191, 859-69]. These results indicate that MAPKAP K2 is not only essential for TNF and IL-1 production but also for signaling induced by cytokines. In addition, MAPKAP K2 and K3 phosphorylate and thus regulate heat shock proteins HSP 25 and HSP 27, which are involved in cytoskeletal reorganization.

Several small molecule inhibitors of p38 have been reported which inhibit IL-1 and TNF synthesis in human monocytes at concentrations in the low μM range [Lee, J. C. et al, Int. J. Immunopharm. 1988, 10, 835] and exhibit activity in animal models which are refractory to cyclooxygenase inhibitors [Lee, J. C. et al, Annals N.Y. Acad. Sci. 1993, 696, 149]. In addition, these small molecule inhibitors are known to also decrease the synthesis of a wide variety of pro-inflammatory proteins including IL-6, IL-8, granulocyte/macrophage colony-stimulating factor (GM-CSF) and cyclooxygenase-2 (COX-2). TNF-induced phosphorylation and activation of cytosolic PLA2, TNF-induced expression of VCAM-1 on endothelial cells, and IL-1-stimulated synthesis of collagenase and stromelysin are also inhibited by such small molecule inhibitors of p38 [Cohen, P. Trends Cell Biol. 1997, 7, 353-61].

A variety of cells including monocytes and macrophages produce TNF and IL-1. Excessive or unregulated TNF production is implicated in a number of disease states including Crohn's disease, ulcerative colitis, pyresis, rheumatoid arthritis, rheumatoid spondylitis, osteoarthritis, gouty arthritis and other arthritic conditions, toxic shock syndrome, endotoxic shock, sepsis, septic shock, gram negative sepsis, bone resorption diseases, reperfusion injury, graft vs. host reaction, allograft rejection, adult respiratory distress syndrome, chronic pulmonary inflammatory disease, silicosis, pulmonary sarcoidosis, cerebral malaria, scar tissue formation, keloid formation, fever and myalgias due to infection such as influenza, cachexia secondary to acquired immune deficiency syndrome (AIDS), cachexia secondary to infection or malignancy, AIDS or AIDS-related complex.

The central position that p38 occupies within the cascade of signaling molecules mediating extracellular-to-intracellular signaling, and its influence over not only IL-1, TNF and IL-8 production but also the synthesis and/or action of other pro-inflammatory proteins (e.g. IL-6, GM-CSF, COX-2, collagenase and stromelysin), make it an attractive target for inhibition by small molecule inhibitors with the expectation that such inhibition would be a highly effective mechanism for regulating the excessive and destructive activation of the immune system. Such an expectation is supported by the potent and diverse anti-inflammatory activities described for p38 kinase inhibitors [Adams, ibid; Badger, et al, J. Pham. Exp. Ther. 1996, 279, 1453-61; Griswold, et al, Pharmacol. Comm., 1996, 7, 323-29].

### SUMMARY OF THE INVENTION

Novel compounds and pharmaceutical compositions that ameliorate imflammatory and immune disorders by inhibiting p38 kinase and the isoforms and splice variants thereof, especially p38α and p38β have been found, together with methods of synthesizing and using the compounds, including methods for inhibiting p38 kinase in a patient by administering the compounds.

The present invention discloses a class of compounds, useful in treating p38 kinase mediated disorders and conditions, defined by structural Formula I:

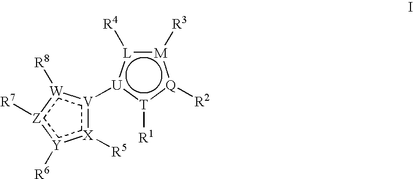

or a salt, ester, tautomer or prodrug thereof, wherein:

L, M, T, X and Y are each independently selected from the group consisting of N, C, O and S;

Q, U, V and W are each independently selected from the group consisting of N and C;

Z is selected from the group consisting of N, C(O), C, O and S;

R¹ is selected from the group consisting of alkoxy, lower alkyl, lower alkylacyl, lower alkylalkoxy, lower alkylether, amide, amino, lower aminoalkyl, halo, hydrogen, hydroxy and null, any of which may be optionally substituted;

R² is selected from the group consisting of —C(O)R⁹, —C(S)(NR¹⁰R¹¹), —C[N(OR¹²)]R¹³, —C(NR¹⁴)(NR¹⁰R¹¹) and —S(O)ₙR¹⁵;

n is 0, 1 or 2;

R³ is selected from the group consisting of alkoxy, lower alkyl, lower alkylether, amino, lower aminoalkyl, halo, haloalkyl, hydrogen, hydroxy and null, any of which may be optionally substituted;

R⁴ is selected from the group consisting of lower alkyl, halo, haloalkyl, hydrogen and null, any of which may be optionally substituted;

R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null, O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;

R⁷ is selected from the group consisting of lower alkylacyl, lower alkyl, lower alkylether, halo, hydrogen, hydroxy, lower hydroxyalkyl and null, any of which may be optionally substituted;

R⁸ is selected from the group consisting of aryl and heteroaryl, either of which may be optionally substituted;

R⁹ is selected from the group consisting of NR¹⁶R¹⁷, OR¹⁸, SR¹⁹, lower alkyl, lower alkenyl, alkynyl, amino, lower aminoalkyl, aralkyl, aryl, arylamino, arylcarbonyl, arylthio, arylsulfonyl, carbonylalkyl, carboxy, cycloalkyl, cycloalkylalkyl, cycloalkenyl, cycloalkylamino, haloalkyl, heteroaralkyl, heterocycloalkyl, heterocycloalkylalkyl, hydrogen, hydroxyalkyl, O-carbamoyl and N-carbamoyl, any of which may be optionally substituted;

R¹⁰, R¹¹, R¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted;

R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;

R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and

R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

The invention further provides compounds of the Formula II:

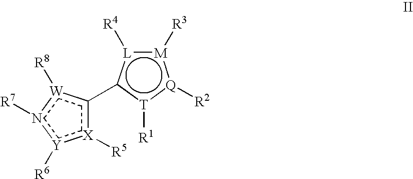

wherein:

R¹ is selected from the group consisting of lower alkyl, lower acylalkyl, lower alkoxy, amide, amino, lower aminoalkyl, lower alkylether, halo, hydrogen, hydroxy, hydroxyalkyl and null, any of which may be optionally substituted;

R² is selected from the group consisting of —C(O)R⁹, —C[N(OR¹²)]R¹³ and —S(O)ₙR¹⁵;

n is 0, 1 or 2;

R³ is selected from the group consisting of lower alkyl, lower aminoalkyl, halo, lower haloalkyl, hydrogen, hydroxy and null, any of which may be optionally substituted;

R⁴ is selected from the group consisting of lower alkyl, halo, hydrogen and null, any of which may be optionally substituted;

R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null, O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;

R⁷ is selected from the group consisting of acyl, lower alkyl, lower alkylether, hydrogen, hydroxy, hydroxyalkyl and null, any of which may be optionally substituted;

R⁸ is selected from the group consisting of aryl and heteroaryl, either of which may be optionally substituted;

R⁹ is selected from the group consisting of NR¹⁶R¹⁷, OR¹⁸, SR¹⁹, lower alkyl, lower alkenyl, lower alkynyl, lower aminoalkyl, aralkyl, aryl, arylamino, arylcarbonyl, lower carbonylalkyl, heteroaralkyl, hydrogen and thioalkyl, any of which may be optionally substituted;

R¹⁰, R¹¹, R¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted;

R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;

R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and

R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

The invention further provides compounds of the Formula III:

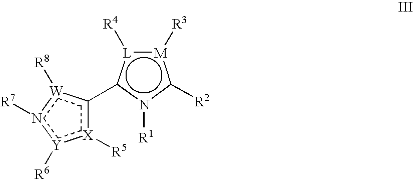

wherein:

R¹ is selected from the group consisting of lower alkoxy, lower alkyl, halo, hydrogen, hydroxy and null, any of which may be optionally substituted;

R² is selected from the group consisting of —C(O)R⁹ and —S(O)ₙR¹⁵;

n is 0, 1 or 2;

R³ is selected from the group consisting of lower alkoxy, lower alkyl, halo, hydrogen, hydroxy and null, any of which may be optionally substituted;

R⁴ is selected from the group consisting of lower alkyl, halo, haloalkyl, hydrogen and null, any of which may be optionally substituted;

R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null. O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;

R⁷ is selected from the group consisting of lower acyl, lower alkyl, halo, hydrogen, hydroxyl and null, any of which may be optionally substituted;

R⁸ is selected from the group consisting of aryl and heteroaryl, either of which may be optionally substituted;

R⁹ is selected from the group consisting of NR¹⁶R¹⁷, OR¹⁸, SR¹⁹, lower alkyl, lower alkenyl, alkynyl, amino, lower aminoalkyl, aralkyl, aryl, arylamino, arylcarbonyl, arylthio, arylsulfonyl, carbonylalkyl, carboxy, cycloalkyl, cycloalkylalkyl, cycloalkenyl, cycloalkylamino, haloalkyl, heteroaralkyl, heterocycloalkyl, heterocycloalkylalkyl, hydrogen, hydroxyalkyl, O-carbamoyl and N-carbamoyl, any of which may be optionally substituted;

R¹⁰, R¹¹, R¹¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted;

R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;

R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and

R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

The invention further provides compounds of the Formula IV:

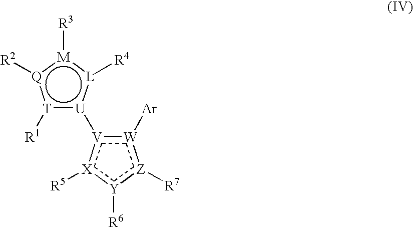

or a salt, ester, tautomer or prodrug thereof, wherein:

R¹ is selected from the group consisting of lower alkyl, lower alkylacyl, amide, amino, lower aminoalkyl, lower alkyl ether, halo, hydrogen, hydroxy, hydroxyalkyl and null;

R² may be selected from the group consisting of —C(O)R⁸, —C(S)NR⁹R¹⁰, —C[N(OR¹¹)]R⁸, C(NR¹²)(NR⁹R¹⁰) and —S(O)ₙR⁸;

n is 0, 1, or 2;

R³ is selected from the group consisting of lower alkyl, lower alkyl ether, amino, lower aminoalkyl, halo, lower haloalkyl, hydrogen, hydroxy, hydroxyalkyl and null;

R⁴ is selected from the group consisting of lower alkyl, halo, lower haloalkyl, hydrogen and null;

R⁵ and R⁶ are independently selected from the group consisting of amino, lower aminoalkyl, carbamoyl, carboxy, cyano, formyl, guanidino, halo, hydroxy, hydrogen, nitro, null, trifluoromethyl, trifluoromethoxy, ureido, C₁₋₈ alkyl, C₁₋₈ alkoxy, C₃₋₈ cycloalkoxyl, C₄₋₈ alkylcycloalkoxy, C₁₋₈ alkylcarbonyl, C₁₋₈ alkoxycarbonyl, N—C₁₋₄ alkylcarbamoyl, N,N-di-[C₁₋₄ alkyl]carbamoyl, hydroxyamino, C₄ alkoxyamino, C₂₋₄ alkanoyloxyamino, C₁₋₄ alkylamino, di[C₁₋₄ alkyl]amino, di-[C₁₋₄ alkyl]amino-C₁₋₄ alkylene-(C₁₋₄ alkyl)amino, C₁₋₄ alkylamino-C₁₋₄ alkylene-(C₁₋₄ alkyl)amino, hydroxy-C₁₋₄ alkylene-(C₁₋₄ alkyl)amino, phenyl, phenoxy, 4-pyridon-1-yl, pyrrolidin-1-yl, imidazol-1-yl, piperidino, morpholino, thiomorpholino, thiomorpholino-1-oxide, thiomorpholino-1,1-dioxide, piperazin-1-yl, 4-C₁₋₄ alkylpiperazin-1-yl, dioxolanyl, C₁₋₈ alkylthio, arylthio, C₁₋₄ alkylsulphinyl, C₁₋₄ alkylsulphonyl, arylsulphonyl, arylsulphonyl, halogen O—C₁₋₄ alkyl, hydroxy-C₁₋₄ alkyl, C₂₋₄ alkanoyloxy-C₁₋₄ alkyl, C₁₋₄ alkoxy-C₁₋₄ alkyl, carboxy-C₁₋₄ alkyl, formyl-C₁₋₄ alkyl, C₁₋₄ alkoxycarbonyl-C₁₋₄-alkyl, carbamoyl-C₁₋₄ alkyl, N—C₁₋₄ alkylcarbamoyl-C₁₋₄ alkyl, N,N-di-[C₁₋₄ alkyl]carbamoyl-C₁₋₄ alkyl, amino-C₁₋₄ alkyl, C₁₋₄ alkylamino-C₁₋₄ alkyl, di-[C₁₋₄ alkyl]amino-C₁₋₄ alkyl, phenyl-C₁₋₄ alkyl, 4-pyridon-1-yl-C₁₋₄ alkyl, pyrrolidin-1-yl-C₁₋₄ alkyl, imidazol-1-yl-C₁₋₄ alkyl, piperidino-C₁₋₄ alkyl, morpholino-C₁₋₄ alkyl, thiomorpholino-C₁₋₄ alkyl, thiomorpholino-1-oxide-C₁₋₄ alkyl, thiomorpholino-1,1-dioxide-C₁₋₄ alkyl, piperazin-1-yl-C₁₋₄ alkyl, 4-C₁₋₄ alkylpiperazin-1-yl-C₁₋₄ alkyl, hydroxy-C₂₋₄ alkoxy-C₁₋₄ alkyl, C₁₋₄ alkoxy-C₂₋₄ alkoxy-C₁₋₄ alkyl, hydroxy-C₂₋₄ alkylamino-C₁₋₄ alkyl, C₁₋₄ alkoxy-C₂₋₄ alkylamino-C₁₋₄ alkyl, C₁₋₄ alkylthio-C₁₋₄ alkyl, C₁₋₄ alkylsulphinyl-C₁₋₄ alkyl, C₁₋₄ alkylsulphonyl-C₁₋₄ alkyl, hydroxy-C₂₋₄ alkylthio-C₁₋₄ alkyl, C₁₋₄ alkoxy-C₂₋₄ alkylthio-C₁₋₄ alkyl, phenoxy-C₁₋₄ alkyl, anilino-C₁₋₄ alkyl, phenylthio-C₁₋₄ alkyl, cyano-C₁₋₄ alkyl, halogen O—C₂₋₄ alkoxy, hydroxy-C₂₋₄ alkoxy, C₂₋₄ alkanoyloxy-C₂₋₄ alkoxy, C₁₋₄ alkoxy-C₂₋₄ alkoxy, carboxy-C₁₋₄ alkoxy, formyl-C₁₋₄ alkoxy, C₁₋₄ alkoxycarbonyl-C₁₋₄ alkoxy, carbamoyl-C₁₋₄ alkoxy, N—C₁₋₄ alkylcarbamoyl-C₁₋₄ alkoxy, N,N-di-[C₁₋₄ alkyl]carbamoyl-C₁₋₄ alkoxy, amino-C₂₋₄ alkoxy, C₁₋₄ alkylamino-C₂₋₄ alkoxy, di-[C₁₋₄ alkyl]amino-C₂₋₄ alkoxy, di-[C₁₋₄ alkyl-C₂₋₄ alkoxy]amino-C₂₋₄ alkoxy, C₂₋₄ alkanoyloxy, hydroxy-C₂₋₄ alkanoyloxy, C₁₋₄ alkoxy-C₂₋₄ alkanoyloxy, phenyl-C₁₋₄ alkoxy, phenoxy-C₂₋₄ alkoxy, anilino-C₂₋₄ alkoxy, phenylthio-C₂₋₄ alkoxy, 4-pyridin-1-yl-C₂₋₄ alkoxy, piperidino-C₂₋₄ alkoxy, morpholino-C₂₋₄ alkoxy, thiomorpholino-C₂₋₄ alkoxy, thiomorpholino-1-oxide-C₂₋₄ alkoxy, thiomorpholino-1,1-dioxide-C₂₋₄ alkoxy, piperazin-1-yl-C₂₋₄ alkoxy, 4-C₁₋₄ alkylpiperazin-1-yl-C₂₋₄ alkoxy, pyrrolidin-1-yl-C₂₋₄ alkoxy, imidazol-1-yl-C₂₋₄ alkoxy, halogeno-C₂₋₄ alkylamino, hydroxy-C₂₋₄ alkylamino, C₂₋₄ alkanoyloxy-C₂₋₄ alkylamino, C₁₋₄ alkoxy-C₂₋₄ alkylamino, carboxy-C₁₋₄ alkylamino, C₁₋₄ alkoxycarbonyl-C₁₋₄ alkylamino, carbamoyl-C₁₋₄ alkylamino, N—C₁₋₄ alkylcarbamoyl-C₁₋₄ alkylamino, N,N-di-[C₁₋₄ alkyl]carbamoyl-C₁₋₄ alkylamino, amino-C₂₋₄ alkylamino, C₁₋₄ alkylamino-C₂₋₄ alkylamino, di-[C₁₋₄ alkyl]amino-C₂₋₄ alkylamino, phenyl-C₁₋₄ alkylamino, phenoxy-C₂₋₄ alkylamino, anilino-C₂₋₄ alkylamino, 4-pyridon-1-yl-C₂₋₄ alkylamino, pyrrolidin-1-yl-C₂₋₄ alkylamino, imidazol-1-yl-C₂₋₄ alkylamino, piperidino-C₂₋₄ alkylamino, morpholino-C₂₋₄ alkylamino, thiomorpholino-C₂₋₄ alkylamino, thiomorpholino-1-oxide-C₂₋₄ alkylamino, thiomorpholino-1,1-dioxide-C₂₋₄ alkylamino, piperazin-1-yl-C₂₋₄ alkylamino, 4-(C₁₋₄ alkyl)piperazin-1-yl-C₂₋₄ alkylamino, phenylthio-C₂₋₄ alkylamino, C₂₋₄ alkanoylamino, C₁₋₄ alkoxycarbonylamino, C₁₋₄ alkylsulphonylamino, C₁₋₄ alkylsulphinylamino, benzamido, benzenesulphonamido, 3-phenylureido, 2-oxopyrrolidin-1-yl, 2,5-dioxopyrrolidin-1-yl, halogeno-C₂₋₄ alkanoylamino, hydroxy-C₂₋₄ alkanoylamino, hydroxy-C₂₋₄ alkanoyl-(C₁₋₄ alkyl)-amino, C₁₋₄ alkoxy-C₂₋₄ alkanoylamino, carboxy-C₂₋₄ alkanoylamino, C₁₋₄ alkoxycarbonyl-C₂₋₄ alkanoylamino, carbamoyl-C₂₋₄ alkanoylamino, N—C₁₋₄ alkylcarbamoyl-C₂₋₄ alkanoylamino, N,N-di-[C₁₋₄ alkyl]carbamoyl-C₂₋₄ alkanoylamino, amino-C₂₋₄ alkanoylamino, C₁₋₄ alkylamino-C₂₋₄ alkanoylamino and di-[C₁₋₄ alkyl]amino-C₂₋₄ alkanoylamino; and any of which may be optionally substituted with one or more radicals independently selected from lower acylalkyl, lower alkoxy, lower alkyl, lower alkylacyl, lower aminoalkyl, amino, lower aminoalkyl, cyano, halo, haloalkyl, hydroxy, lower hydroxyalkyl and nitro, or R⁵ and R⁶ may combine to form an optionally substituted heterocycloalkyl or heteroaryl;

R⁷ is selected from the group consisting of lower alkyl, lower alkylacyl, lower alkyl ether, halo, hydrogen, hydroxyl, lower hydroxyalkyl and null, any of which may be optionally substituted with one or more radicals independently selected from alkoxy, lower alkyl, lower alkylacyl, amino, cyano, halo, haloalkyl, hydroxy and nitro;

R⁸ is selected from the group consisting of NR⁹R¹⁰, OR⁹, SR⁹, alkoxyalkyl, lower alkyl, lower alkenyl, lower alkynyl, aralkyl, lower aminoalkyl, arylaminocarbonylalkyl, aminocarbonylalkyl, arylaminocarbonylalkyl, arylcarbonylalkyl, alkylthioalkyl, cycloalkylthioalkyl, arylsulfonylaminoalkyl, carbonylalkyl, carbonylheterocyclylcarbonylalkyl, cycloalkylalkyl, cycloalkenylalkyl, haloalkyl, hydroxyalkyl and heterocycloalkyl, any of which may be optionally substituted with one or more radicals independently selected from alkoxy, lower alkyl, lower alkylacyl, amino, lower aminoalkyl, cyano, halo, haloalkyl, hydroxy and nitro;

R⁹ and R¹⁰ are independently selected from the group consisting of lower alkyl, lower alkenyl, lower alkynyl, aralkyl, arylsulfonylaminoalkyl, alkoxyalkyl, lower aminoalkyl, arylaminocarbonylalkyl, aminocarbonylalkyl, arylaminocarbonylalkyl, arylcarbonylalkyl, alkylthioalkyl, carbonylalkyl, carbonylheterocyclylcarbonylalkyl, cycloalkyl, cycloalkylalkyl, cycloalkenylalkyl, cycloalkylthioalkyl, haloalkyl, heterocyclylalkyl, heterocyclylalkyl, hydroxyalkyl and hydrogen, any of which may be optionally substituted with one or more radicals independently selected from alkoxy, lower alkylacyl, amino, lower aminoalkyl, lower alkyl, cyano, halo, haloalkyl, hydroxy and nitro; or R⁹ and R¹⁰ may combine to form an optionally substituted heterocycloalkyl or heteroaryl;

R¹¹ is selected from the group consisting of lower alkyl, lower alkenyl, lower alkynyl, aralkyl, cycloalkyl, cycloalkylalkyl, haloalkyl, heteroaralkyl, heterocyloalkyl and hydrogen, any of which may be optionally substituted with one or more radicals independently selected from alkoxy, lower alkylacyl, amino, lower aminoalkyl, lower alkyl, cyano, halo, haloalkyl, hydroxy and nitro;

R¹² is selected from the group consisting of lower alkyl, lower alkenyl, lower alkynyl, haloalkyl, heteroaralkyl, aralkyl, cycloalkyl, cycloalkylalkyl, heterocyloalkyl and hydrogen, any of which may be optionally substituted with one or more radicals independently selected from alkoxy, lower alkylacyl, amino, lower aminoalkyl, lower alkyl, cyano, halo, haloalkyl, hydroxy and nitro;

Ar is selected from the group consisting of aryl and heteroaryl, each optionally substituted with one or more radicals independently selected from lower alkenyl, lower alkynyl, lower alkoxy, alkoxyalkyl, amino, lower aminoalkyl and aminocarbonyl, lower alkylacyl, lower alkyl, lower alkyl amide, carboxy, halo, lower haloalkyl, hydroxy, hydroxyalkyl and hydrogen, any of which may be optionally substituted with one or more radicals independently selected from alkoxy, lower alkylacyl, amino, lower aminoalkyl, lower alkyl, cyano, halo, haloalkyl, hydroxy and nitro;

L, M, T, X and Y are each independently selected from the group consisting of N, C, O and S;

Q, U, V and W are each independently selected from the group consisting of N or C;

Z is selected from the group consisting of N, C(O), C, O and S;

wherein V, W, X, Y and Z taken together form an unsaturated ring containing at least one carbon atom;

with the proviso that when V and W are carbon, then X, Y, and Z are not all nitrogen;

with the proviso that when VWXZY does not form an aromatic ring, then Z must be C(O); with the proviso that when Y is oxygen then R⁷ is null and Ar is not an unsubstituted phenyl or a chloro-monosubstituted phenyl; and

with the proviso that when Z is C, then R⁷ is not hydrogen.

The invention further provides compounds of the Formula V:

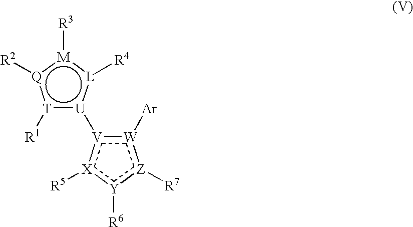

or a salt, ester, tautomer or prodrug thereof, wherein:

R¹ is selected from the group consisting of lower alkyl, lower alkylacyl, amide, lower aminoalkyl, lower alkyl ether, halo, hydrogen, hydroxy, hydroxyalkyl and null;

R² may be selected from the group consisting of —C(O)R⁸, —C[N(OR¹¹)]R⁸ and —S(O)ₙR⁸;

n is 0, 1, or 2;

R³ is selected from the group consisting of lower alkyl, amino, lower aminoalkyl, halo, lower haloalkyl, hydrogen, hydroxy and null;

R⁴ is selected from the group consisting of lower alkyl, chlorine, fluorine, hydrogen and null;

R⁵ and R⁶ are independently selected from the group consisting of amino, lower aminoalkyl, carbamoyl, carboxy, cyano, formyl, guanidino, halo, hydroxy, hydrogen, nitro, null, trifluoromethyl, trifluoromethoxy, ureido, C₁₋₈ alkyl, C₁₋₈ alkoxy, C₃₋₈ cycloalkoxyl, C₄₋₈ alkylcycloalkoxy, C₁₋₈ alkylcarbonyl, C₁₋₈ alkoxycarbonyl, N—C₁₋₄ alkylcarbamoyl, N,N-di-[C₁₋₄ alkyl]carbamoyl, hydroxyamino, C₁₋₄ alkoxyamino, C₂₋₄ alkanoyloxyamino, C₁₋₄ alkylamino, di[C₁₋₄ alkyl]amino, di-[C₁₋₄ alkyl]amino-C₁₋₄ alkylene-(C₁₋₄ alkyl)amino, C₁₋₄ alkylamino-C₁₋₄ alkylene-(C₁₋₄ alkyl)amino, hydroxy-C₁₋₄ alkylene-(C₁₋₄ alkyl)amino, phenyl, phenoxy, 4-pyridon-1-yl, pyrrolidin-1-yl, imidazol-1-yl, piperidino, morpholino, thiomorpholino, thiomorpholino-1-oxide, thiomorpholino-1,1-dioxide, piperazin-1-yl, 4-C₁₋₄ alkylpiperazin-1-yl, dioxolanyl, C₁₋₈ alkylthio, arylthio, C₁₋₄ alkylsulphinyl, C₁₋₄ alkylsulphonyl, arylsulphonyl, arylsulphonyl, halogen O—C₁₋₄ alkyl, hydroxy-C₁₋₄ alkyl, C₂₋₄ alkanoyloxy-C₁₋₄ alkyl, C₁₋₄ alkoxy-C₁₋₄ alkyl, carboxy-C₁₋₄ alkyl, formyl-C₁₋₄ alkyl, C₁₋₄ alkoxycarbonyl-C₁₋₄-alkyl, carbamoyl-C₁₋₄ alkyl, N—C₁₋₄ alkylcarbamoyl-C₁₋₄ alkyl, N,N-di-[C₁₋₄ alkyl]carbamoyl-C₁₋₄ alkyl, amino-C₁₋₄ alkyl, C₁₋₄ alkylamino-C₁₋₄ alkyl, di-[C₁₋₄ alkyl]amino-C₁₋₄ alkyl, phenyl-C₁₋₄ alkyl, 4-pyridon-1-yl-C₁₋₄ alkyl, pyrrolidin-1-yl-C₁₋₄ alkyl, imidazol-1-yl-C₁₋₄ alkyl, piperidino-C₁₋₄ alkyl, morpholino-C₁₋₄ alkyl, thiomorpholino-C₁₋₄ alkyl, thiomorpholino-1-oxide-C₁₋₄ alkyl, thiomorpholino-1,1-dioxide-C₁₋₄ alkyl, piperazin-1-yl-C₁₋₄ alkyl, 4-C₁₋₄ alkylpiperazin-1-yl-C₁₋₄ alkyl, hydroxy-C₂₋₄ alkoxy-C₁₋₄ alkyl, C₁₋₄ alkoxy-C₂₋₄ alkoxy-C₁₋₄ alkyl, hydroxy-C₂₋₄ alkylamino-C₁₋₄ alkyl, C₁₋₄ alkoxy-C₂₋₄ alkylamino-C₁₋₄ alkyl, C₁₋₄ alkylthio-C₁₋₄ alkyl, C₁₋₄ alkylsulphinyl-C₁₋₄ alkyl, C₁₋₄ alkylsulphonyl-C₁₋₄ alkyl, hydroxy-C₂₋₄ alkylthio-C₁₋₄ alkyl, C₁₋₄ alkoxy-C₂₋₄ alkylthio-C₁₋₄ alkyl, phenoxy-C₁₋₄ alkyl, anilino-C₁₋₄ alkyl, phenylthio-C₁₋₄ alkyl, cyano-C₁₋₄ alkyl, halogen O—C₂₋₄ alkoxy, hydroxy-C₂₋₄ alkoxy, C₂₋₄ alkanoyloxy-C₂₋₄ alkoxy, C₁₋₄ alkoxy-C₂₋₄ alkoxy, carboxy-C₁₋₄ alkoxy, formyl-C₁₋₄ alkoxy, C₁₋₄ alkoxycarbonyl-C₁₋₄ alkoxy, carbamoyl-C₄ alkoxy, N—C₁₋₄ alkylcarbamoyl-C₁₋₄ alkoxy, N,N-di-[C₁₋₄ alkyl]carbamoyl-C₁₋₄ alkoxy, amino-C₂₋₄ alkoxy, C₁₋₄ alkylamino-C₂₋₄ alkoxy, di-[C₁₋₄ alkyl]amino-C₂₋₄ alkoxy, di-[C₁₋₄ alkyl-C₂₋₄ alkoxy]amino-C₂₋₄ alkoxy, C₂₋₄ alkanoyloxy, hydroxy-C₂₋₄ alkanoyloxy, C₁₋₄ alkoxy-C₂₋₄ alkanoyloxy, phenyl-C₁₋₄ alkoxy, phenoxy-C₂₋₄ alkoxy, anilino-C₂₋₄ alkoxy, phenylthio-C₂₋₄ alkoxy, 4-pyridin-1-yl-C₂₋₄ alkoxy, piperidino-C₂₋₄ alkoxy, morpholino-C₂₋₄ alkoxy, thiomorpholino-C₂₋₄ alkoxy, thiomorpholino-1-oxide-C₂₋₄ alkoxy, thiomorpholino-1,1-dioxide-C₂₋₄ alkoxy, piperazin-1-yl-C₂₋₄ alkoxy, 4-C₁₋₄ alkylpiperazin-1-yl-C₂₋₄ alkoxy, pyrrolidin-1-yl-C₂₋₄ alkoxy, imidazol-1-yl-C₂₋₄ alkoxy, halogeno-C₂₋₄ alkylamino, hydroxy-C₂₋₄ alkylamino, C₂₋₄ alkanoyloxy-C₂₋₄ alkylamino, C₁₋₄ alkoxy-C₂₋₄ alkylamino, carboxy-C₁₋₄ alkylamino, C₁₋₄ alkoxycarbonyl-C₁₋₄ alkylamino, carbamoyl-C₁₋₄ alkylamino, N—C₁₋₄ alkylcarbamoyl-C₁₋₄ alkylamino, N,N-di-[C₁₋₄ alkyl]carbamoyl-C₁₋₄ alkylamino, amino-C₂₋₄ alkylamino, C₁₋₄ alkylamino-C₂₋₄ alkylamino, di-[C₁₋₄ alkyl]amino-C₂₋₄ alkylamino, phenyl-C₁₋₄ alkylamino, phenoxy-C₂₋₄ alkylamino, anilino-C₂₋₄ alkylamino, 4-pyridon-1-yl-C₂₋₄ alkylamino, pyrrolidin-1-yl-C₂₋₄ alkylamino, imidazol-1-yl-C₂₋₄ alkylamino, piperidino-C₂₋₄ alkylamino, morpholino-C₂₋₄ alkylamino, thiomorpholino-C₂₋₄ alkylamino, thiomorpholino-1-oxide-C₂₋₄ alkylamino, thiomorpholino-1,1-dioxide-C₂₋₄ alkylamino, piperazin-1-yl-C₂₋₄ alkylamino, 4-(C₁₋₄ alkyl)piperazin-1-yl-C₂₋₄ alkylamino, phenylthio-C₂₋₄ alkylamino, C₂₋₄ alkanoylamino, C₁₋₄ alkoxycarbonylamino, C₁₋₄ alkylsulphonylamino, C₁₋₄ alkylsulphinylamino, benzamido, benzenesulphonamido, 3-phenylureido, 2-oxopyrrolidin-1-yl, 2,5-dioxopyrrolidin-1-yl, halogeno-C₂₋₄ alkanoylamino, hydroxy-C₂₋₄ alkanoylamino, hydroxy-C₂₋₄ alkanoyl-(C₁₋₄ alkyl)-amino, C₁₋₄ alkoxy-C₂₋₄ alkanoylamino, carboxy-C₂₋₄ alkanoylamino, C₁₋₄ alkoxycarbonyl-C₂₋₄ alkanoylamino, carbamoyl-C₂₋₄ alkanoylamino, N—C₁₋₄ alkylcarbamoyl-C₂₋₄ alkanoylamino, N,N-di-[C₁₋₄ alkyl]carbamoyl-C₂₋₄ alkanoylamino, amino-C₂₋₄ alkanoylamino, C₁₋₄ alkylamino-C₂₋₄ alkanoylamino and di-[C₁₋₄ alkyl]amino-C₂₋₄ alkanoylamino; any of which may be optionally substituted with one or more radicals independently selected from alkoxy, lower alkylacyl, amino, lower aminoalkyl, lower alkyl, cyano, halo, haloalkyl, hydroxy and nitro, or R⁵ and R⁶ may combine to form an optionally substituted heterocycloalkyl or heteroaryl;

R⁷ is selected from the group consisting of lower alkyl ether, hydroxy, hydrogen and null, any of which may be optionally substituted with one or more radicals independently selected from lower alkoxy, lower alkylacyl, lower alkyl, lower acylalkyl, amino, lower aminoalkyl, lower aminoalkyl, cyano, halo, haloalkyl, hydroxy, lower hydroxyalkyl and nitro;

R⁸ is selected from the group consisting of NR⁹R¹⁰, OR⁹, SR⁹, lower alkyl, lower alkenyl, lower alkynyl, aralkyl, lower aminoalkyl, arylaminocarbonylalkyl, aminocarbonylalkyl, arylaminocarbonylalkyl, arylcarbonylalkyl, carbonylalkyl and haloalkyl, any of which may be optionally substituted with one or more radicals independently selected from alkoxy, lower alkylacyl, amino, lower aminoalkyl, lower alkyl, cyano, halo, haloalkyl, hydroxy and nitro;

R⁹ and R¹⁰ are independently selected from the group consisting of lower alkyl, lower alkenyl, lower alkynyl, aralkyl, alkoxyalkyl, arylsulfonylaminoalkyl, lower aminoalkyl, arylaminocarbonylalkyl, aminocarbonylalkyl, arylaminocarbonylalkyl, arylcarbonylalkyl, alkylthioalkyl, carbonylalkyl, carbonylheterocyclylcarbonylalkyl, cycloalkyl, cycloalkylalkyl, cycloalkenylalkyl, cycloalkylthioalkyl, haloalkyl, hydroxyalkyl, heterocyclylalkyl, heterocyclylalkyl and hydrogen, any of which may be optionally substituted with one or more radicals independently selected from alkoxy, lower alkylacyl, amino, lower aminoalkyl, lower alkyl, cyano, halo, haloalkyl, hydroxy and nitro; or R⁹ and R¹⁰ may combine to form an optionally substituted heterocycloalkyl or heteroaryl;

R¹¹ is selected from the group consisting of lower alkyl, lower alkenyl, lower alkynyl, aralkyl, cycloalkyl, cycloalkylalkyl, haloalkyl, heterocycloalkyl and hydrogen, any of which may be optionally substituted with one or more radicals independently selected from alkoxy, alkyl, lower alkylacyl, amino, lower aminoalkyl, cyano, halo, haloalkyl, hydroxy and nitro;

R¹² is selected from the group consisting of lower alkyl, lower alkenyl, lower alkynyl, aralkyl, cycloalkyl, cycloalkylalkyl, haloalkyl, heteroaralkyl, heterocyloalkyl and hydrogen, any of which may be optionally substituted with one or more radicals independently selected from alkoxy, lower alkyl, lower alkylacyl, amino, lower aminoalkyl, cyano, halo, haloalkyl, hydroxy and nitro;

Ar is selected from the group consisting of phenyl and 5 or 6-membered heteroaryl, any of which may be optionally substituted with one or more radicals independently selected from lower alkenyl, lower alkynyl, lower alkoxy, alkoxyalkyl, amino, lower aminoalkyl, lower alkylaminocarbonyl, lower alkylcarbonylamino, lower alkylacyl, lower alkyl, carboxy, halo, haloalkyl, hydroxy, hydroxyalkyl and hydrogen, any of which may be optionally substituted with one or more radicals independently selected from alkoxy, lower alkylacyl, amino, lower aminoalkyl, lower alkyl, cyano, halo, haloalkyl, hydroxy and nitro;

L, T, X and Y are each independently selected from the group consisting of N, C, O and S;

M is selected from the group consisting of N, C and S;

Q, U, V and W are each independently selected from the group consisting of N or C;

Z is selected from the group consisting of N, C(O), C and O;

wherein V, W, X, Y and Z taken together form an unsaturated ring containing at least one carbon atom;

with the proviso that when V and W are carbon, then X, Y, and Z are not all nitrogen;

with the proviso that when VWXZY does not form an aromatic ring, then Z must be C(O);

with the proviso that when Y is oxygen, then R⁷ is null and Ar is not an unsubstituted phenyl or a chloro-monosubstituted phenyl; and

with the proviso that when Z is C, then R⁷ is not hydrogen.

The invention further provides compounds of the Formula VI:

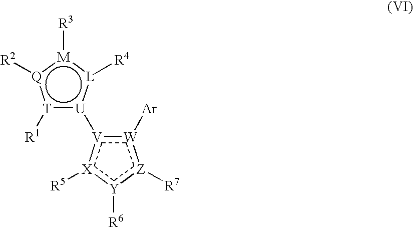

or a salt, ester, tautomer or prodrug thereof, wherein:

R¹ is selected from the group consisting of halo, hydrogen, hydroxy and null;

R² is selected from the group consisting of —C(O)R⁸ or —S(O)ₙR⁸;

n is 0, 1, or 2;

R³ is selected from the group consisting of hydrogen or null;

R⁴ is selected from the group consisting of fluorine, hydrogen and null;

R⁵ is selected from the group consisting of amino, lower aminoalkyl, cyano, halogen, hydroxy, hydrogen, null, trifluoromethoxy, C₁₋₈ alkyl, C₁₋₈ alkoxy, C₃₋₈ cycloalkoxyl, C₄₋₈ alkylcycloalkoxy, hydroxyamino, C₁₋₄ alkoxyamino, C₂₋₄ alkanoyloxyamino, C₁₋₄ alkylamino, imidazol-1-yl, piperidino, morpholino, thiomorpholino, thiomorpholino-1-oxide, thiomorpholino-1,1-dioxide, piperazin-1-yl, 4-C₁₋₄ alkylpiperazin-1-yl, dioxolanyl, C₁₋₈ alkylthio, arylthio, C₁₋₄ alkylsulphinyl, halogen O—C₁₋₄ alkyl, hydroxy-C₁₋₄ alkyl, C₂₋₄ alkanoyloxy-C₁₋₄ alkyl, C₁₋₄ alkoxy-C₁₋₄ alkyl, carboxy-C₁₋₄ alkyl, formyl-C₁₋₄ alkyl, C₁₋₄ alkoxycarbonyl-C₁₋₄-alkyl, carbamoyl-C₁₋₄ alkyl, N—C₁₋₄ alkylcarbamoyl-C₁₋₄ alkyl, N,N-di-[C₁₋₄ alkyl]carbamoyl-C₁₋₄ alkyl, amino-C₁₋₄ alkyl, C₁₋₄ alkylamino-C₁₋₄ alkyl, di-[C₁₋₄ alkyl]amino-C₁₋₄ alkyl, phenyl-C₁₋₄ alkyl, 4-pyridon-1-yl-C₁₋₄ alkyl, pyrrolidin-1-yl-C₁₋₄ alkyl, imidazol-1-yl-C₁₋₄ alkyl, piperidino-C₁₋₄ alkyl, morpholino-C₁₋₄ alkyl, thiomorpholino-C₁₋₄ alkyl, thiomorpholino-1-oxide-C₁₋₄ alkyl, thiomorpholino-1,1-dioxide-C₁₋₄ alkyl, piperazin-1-yl-C₁₋₄ alkyl, 4-C₁₋₄ alkylpiperazin-1-yl-C₁₋₄ alkyl, hydroxy-C₂₋₄ alkoxy-C₁₋₄ alkyl, C₁₋₄ alkoxy-C₂₋₄ alkoxy-C₁₋₄ alkyl, hydroxy-C₂₋₄ alkylamino-C₁₋₄ alkyl, C₁₋₄ alkoxy-C₂₋₄ alkylamino-C₁₋₄ alkyl, C₁₋₄ alkylthio-C₁₋₄ alkyl, C₁₋₄ alkylsulphinyl-C₁₋₄ alkyl, C₁₋₄ alkylsulphonyl-C₁₋₄ alkyl, hydroxy-C₂₋₄ alkylthio-C₁₋₄ alkyl, C₁₋₄ alkoxy-C₂₋₄ alkylthio-C₁₋₄ alkyl, phenoxy-C₁₋₄ alkyl, anilino-C₁₋₄ alkyl, phenylthio-C₁₋₄ alkyl, cyano-C₁₋₄ alkyl, halogen O—C₂₋₄ alkoxy, hydroxy-C₂₋₄ alkoxy, C₂₋₄ alkanoyloxy-C₂₋₄ alkoxy, C₁₋₄ alkoxy-C₂₋₄ alkoxy, carboxy-C₁₋₄ alkoxy, formyl-C₁₋₄ alkoxy, C₁₋₄ alkoxycarbonyl-C₁₋₄ alkoxy, carbamoyl-C₁₋₄ alkoxy, N—C₁₋₄ alkylcarbamoyl-C₁₋₄ alkoxy, N,N-di-[C₁₋₄ alkyl]carbamoyl-C₁₋₄ alkoxy, amino-C₂₋₄ alkoxy, C₁₋₄ alkylamino-C₂₋₄ alkoxy, di-[C₁₋₄ alkyl]amino-C₂₋₄ alkoxy, di-[C₁₋₄ alkyl-C₂₋₄ alkoxy]amino-C₂₋₄ alkoxy, C₂₋₄ alkanoyloxy, hydroxy-C₂₋₄ alkanoyloxy, C₁₋₄ alkoxy-C₂₋₄ alkanoyloxy, phenyl-C₁₋₄ alkoxy, phenoxy-C₂₋₄ alkoxy, anilino-C₂₋₄ alkoxy, phenylthio-C₂₋₄ alkoxy, 4-pyridin-1-yl-C₂₋₄ alkoxy, piperidino-C₂₋₄ alkoxy, morpholino-C₂₋₄ alkoxy, thiomorpholino-C₂₋₄ alkoxy, thiomorpholino-1-oxide-C₂₋₄ alkoxy, thiomorpholino-1,1-dioxide-C₂₋₄ alkoxy, piperazin-1-yl-C₂₋₄ alkoxy, 4-C₁₋₄ alkylpiperazin-1-yl-C₂₋₄ alkoxy, pyrrolidin-1-yl-C₂₋₄ alkoxy, imidazol-1-yl-C₂₋₄ alkoxy, halogeno-C₂₋₄ alkylamino, hydroxy-C₂₋₄ alkylamino, C₂₋₄ alkanoyloxy-C₂₋₄ alkylamino, C₁₋₄ alkoxy-C₂₋₄ alkylamino, carboxy-C₁₋₄ alkylamino, C₄ alkoxycarbonyl-C₁₋₄ alkylamino, carbamoyl-C₁₋₄ alkylamino, N—C₁₋₄ alkylcarbamoyl-C₁₋₄ alkylamino, N,N-di-[C₁₋₄ alkyl]carbamoyl-C₁₋₄ alkylamino, amino-C₂₋₄ alkylamino, C₁₋₄ alkylamino-C₂₋₄ alkylamino, di-[C₁₋₄ alkyl]amino-C₂₋₄ alkylamino, phenyl-C₁₋₄ alkylamino, phenoxy-C₂₋₄ alkylamino, anilino-C₂₋₄ alkylamino, 4-pyridon-1-yl-C₂₋₄ alkylamino, pyrrolidin-1-yl-C₂₋₄ alkylamino, imidazol-1-yl-C₂₋₄ alkylamino, piperidino-C₂₋₄ alkylamino, morpholino-C₂₋₄ alkylamino, thiomorpholino-C₂₋₄ alkylamino, thiomorpholino-1-oxide-C₂₋₄ alkylamino, thiomorpholino-1,1-dioxide-C₂₋₄ alkylamino, piperazin-1-yl-C₂₋₄ alkylamino, 4-(C₄ alkyl)piperazin-1-yl-C₂₋₄ alkylamino, phenylthio-C₂₋₄ alkylamino, C₂₋₄ alkanoylamino, C₁₋₄ alkoxycarbonylamino, C₁₋₄ alkylsulphonylamino, C₁₋₄ alkylsulphinylamino, benzamido, benzenesulphonamido, halogeno-C₂₋₄ alkanoylamino, hydroxy-C₂₋₄ alkanoylamino, hydroxy-C₂₋₄ alkanoyl-(C₁₋₄ alkyl)-amino, C₁₋₄ alkoxy-C₂₋₄ alkanoylamino, carboxy-C₂₋₄ alkanoylamino, C₁₋₄ alkoxycarbonyl-C₂₋₄ alkanoylamino, carbamoyl-C₂₋₄ alkanoylamino, N—C₁₋₄ alkylcarbamoyl-C₂₋₄ alkanoylamino, N,N-di-[C₁₋₄ alkyl]carbamoyl-C₂₋₄ alkanoylamino, amino-C₂₋₄ alkanoylamino, C₁₋₄ alkylamino-C₂₋₄ alkanoylamino and di-[C₁₋₄ alkyl]amino-C₂₋₄ alkanoylamino; and any of which may be optionally substituted with one or more radicals independently selected from alkoxy, lower alkylacyl, amino, lower aminoalkyl, lower alkyl, cyano, halo, haloalkyl, hydroxy and nitro;

R⁶ is selected from the group consisting of amino, lower aminoalkyl, carbamoyl, carboxy, cyano, formyl, guanidino, halogen, hydroxy, hydrogen, nitro, null, trifluoromethyl, trifluoromethoxy, ureido, C₁₋₈ alkyl, C₁₋₈ alkoxy, C₃₋₈ cycloalkoxyl, C₄₋₈ alkylcycloalkoxy, C₁₋₈ alkylcarbonyl, C₁₋₈ alkoxycarbonyl, N—C₁₋₄ alkylcarbamoyl, N,N-di-[C₄ alkyl]carbamoyl, hydroxyamino, C₁₋₄ alkoxyamino, C₂₋₄ alkanoyloxyamino, C₁₋₄ alkylamino, di[C₁₋₄ alkyl]amino, di-[C₁₋₄ alkyl]amino-C₁₋₄ alkylene-(C₁₋₄ alkyl)amino, C₁₋₄ alkylamino-C₁₋₄ alkylene-(C₁₋₄ alkyl)amino, hydroxy-C₁₋₄ alkylene-(C₁₋₄ alkyl)amino, phenyl, phenoxy, 4-pyridon-1-yl, pyrrolidin-1-yl, imidazol-1-yl, piperidino, morpholino, thiomorpholino, thiomorpholino-1-oxide, thiomorpholino-1,1-dioxide, piperazin-1-yl, 4-C₁₋₄ alkylpiperazin-1-yl, dioxolanyl, C₁₋₈ alkylthio, arylthio, C₁₋₄ alkylsulphinyl, C₁₋₄ alkylsulphonyl, arylsulphonyl, arylsulphonyl, halogen O—C₁₋₄ alkyl, hydroxy-C₁₋₄ alkyl, C₂₋₄ alkanoyloxy-C₁₋₄ alkyl, C₁₋₄ alkoxy-C₁₋₄ alkyl, carboxy-C₁₋₄ alkyl, formyl-C₁₋₄ alkyl, C₁₋₄ alkoxycarbonyl-C₁₋₄-alkyl, carbamoyl-C₁₋₄ alkyl, N—C₁₋₄ alkylcarbamoyl-C₁₋₄ alkyl, N,N-di-[C₁₋₄ alkyl]carbamoyl-C₁₋₄ alkyl, amino-C₁₋₄ alkyl, C₁₋₄ alkylamino-C₁₋₄ alkyl, di-[C₁₋₄ alkyl]amino-C₁₋₄ alkyl, phenyl-C₁₋₄ alkyl, 4-pyridon-1-yl-C₁₋₄ alkyl, pyrrolidin-1-yl-C₁₋₄ alkyl, imidazol-1-yl-C₁₋₄ alkyl, piperidino-C₁₋₄ alkyl, morpholino-C₁₋₄ alkyl, thiomorpholino-C₁₋₄ alkyl, thiomorpholino-1-oxide-C₁₋₄ alkyl, thiomorpholino-1,1-dioxide-C₁₋₄ alkyl, piperazin-1-yl-C₁₋₄ alkyl, 4-C₁₋₄ alkylpiperazin-1-yl-C₁₋₄ alkyl, hydroxy-C₂₋₄ alkoxy-C₁₋₄ alkyl, C₁₋₄ alkoxy-C₂₋₄ alkoxy-C₁₋₄ alkyl, hydroxy-C₂₋₄ alkylamino-C₁₋₄ alkyl, C₁₋₄ alkoxy-C₂₋₄ alkylamino-C₁₋₄ alkyl, C₁₋₄ alkylthio-C₁₋₄ alkyl, C₁₋₄ alkylsulphinyl-C₁₋₄ alkyl, C₁₋₄ alkylsulphonyl-C₁₋₄ alkyl, hydroxy-C₂₋₄ alkylthio-C₁₋₄ alkyl, C₁₋₄ alkoxy-C₂₋₄ alkylthio-C₁₋₄ alkyl, phenoxy-C₁₋₄ alkyl, anilino-C₁₋₄ alkyl, phenylthio-C₁₋₄ alkyl, cyano-C₁₋₄ alkyl, halogen O—C₂₋₄ alkoxy, hydroxy-C₂₋₄ alkoxy, C₂₋₄ alkanoyloxy-C₂₋₄ alkoxy, C₁₋₄ alkoxy-C₂₋₄ alkoxy, carboxy-C₁₋₄ alkoxy, formyl-C₁₋₄ alkoxy, C₁₋₄ alkoxycarbonyl-C₁₋₄ alkoxy, carbamoyl-C₁₋₄ alkoxy, N—C₁₋₄ alkylcarbamoyl-C₁₋₄ alkoxy, N,N-di-[C₁₋₄ alkyl]carbamoyl-C₁₋₄ alkoxy, amino-C₂₋₄ alkoxy, C₁₋₄ alkylamino-C₂₋₄ alkoxy, di-[C₁₋₄ alkyl]amino-C₂₋₄ alkoxy, di-[C₁₋₄ alkyl-C₂₋₄ alkoxy]amino-C₂₋₄ alkoxy, C₂₋₄ alkanoyloxy, hydroxy-C₂₋₄ alkanoyloxy, C₁₋₄ alkoxy-C₂₋₄ alkanoyloxy, phenyl-C₁₋₄ alkoxy, phenoxy-C₂₋₄ alkoxy, anilino-C₂₋₄ alkoxy, phenylthio-C₂₋₄ alkoxy, 4-pyridin-1-yl-C₂₋₄ alkoxy, piperidino-C₂₋₄ alkoxy, morpholino-C₂₋₄ alkoxy, thiomorpholino-C₂₋₄ alkoxy, thiomorpholino-1-oxide-C₂₋₄ alkoxy, thiomorpholino-1,1-dioxide-C₂₋₄ alkoxy, piperazin-1-yl-C₂₋₄ alkoxy, 4-C₁₋₄ alkylpiperazin-1-yl-C₂₋₄ alkoxy, pyrrolidin-1-yl-C₂₋₄ alkoxy, imidazol-1-yl-C₂₋₄ alkoxy, halogeno-C₂₋₄ alkylamino, hydroxy-C₂₋₄ alkylamino, C₂₋₄ alkanoyloxy-C₂₋₄ alkylamino, C₁₋₄ alkoxy-C₂₋₄ alkylamino, carboxy-C₁₋₄ alkylamino, C₁₋₄ alkoxycarbonyl-C₁₋₄ alkylamino, carbamoyl-C₁₋₄ alkylamino, N—C₁₋₄ alkylcarbamoyl-C₁₋₄ alkylamino, N,N-di-[C₁₋₄ alkyl]carbamoyl-C₁₋₄ alkylamino, amino-C₂₋₄ alkylamino, C₁₋₄ alkylamino-C₂₋₄ alkylamino, di-[C₁₋₄ alkyl]amino-C₂₋₄ alkylamino, phenyl-C₁₋₄ alkylamino, phenoxy-C₂₋₄ alkylamino, anilino-C₂₋₄ alkylamino, 4-pyridon-1-yl-C₂₋₄ alkylamino, pyrrolidin-1-yl-C₂₋₄ alkylamino, imidazol-1-yl-C₂₋₄ alkylamino, piperidino-C₂₋₄ alkylamino, morpholino-C₂₋₄ alkylamino, thiomorpholino-C₂₋₄ alkylamino, thiomorpholino-1-oxide-C₂₋₄ alkylamino, thiomorpholino-1,1-dioxide-C₂₋₄ alkylamino, piperazin-1-yl-C₂₋₄ alkylamino, 4-(C₁₋₄ alkyl)piperazin-1-yl-C₂₋₄ alkylamino, phenylthio-C₂₋₄ alkylamino, C₂₋₄ alkanoylamino, C₁₋₄ alkoxycarbonylamino, C₁₋₄ alkylsulphonylamino, C₁₋₄ alkylsulphinylamino, benzamido, benzenesulphonamido, 3-phenylureido, 2-oxopyrrolidin-1-yl, 2,5-dioxopyrrolidin-1-yl, halogeno-C₂₋₄ alkanoylamino, hydroxy-C₂₋₄ alkanoylamino, hydroxy-C₂₋₄ alkanoyl-(C₁₋₄ alkyl)-amino, C₁₋₄ alkoxy-C₂₋₄ alkanoylamino, carboxy-C₂₋₄ alkanoylamino, C₁₋₄ alkoxycarbonyl-C₂₋₄ alkanoylamino, carbamoyl-C₂₋₄ alkanoylamino, N—C₁₋₄ alkylcarbamoyl-C₂₋₄ alkanoylamino, N,N-di-[C₁₋₄ alkyl]carbamoyl-C₂₋₄ alkanoylamino, amino-C₂₋₄ alkanoylamino, C₁₋₄ alkylamino-C₂₋₄ alkanoylamino and di-[C₁₋₄ alkyl]amino-C₂₋₄ alkanoylamino; and any of which may be optionally substituted with one or more radicals independently selected from lower alkyl, alkoxy, lower alkylacyl, amino, lower aminoalkyl, cyano, halo, haloalkyl, hydroxy and nitro; or R⁵ and R⁶ may combine to form an optionally substituted heterocycloalkyl or heteroaryl;

R⁷ is selected from the group consisting of hydroxy, hydrogen or null;

R⁸ is selected from the group consisting of NR⁹R¹⁰, SR⁹, lower alkyl, lower alkenyl, lower alkynyl, aralkyl, lower aminoalkyl, carbonylalkyl and haloalkyl, any of which may be optionally substituted with one or more radicals independently selected from alkoxy, lower alkylacyl, amino, lower aminoalkyl, lower alkyl, cyano, halo, haloalkyl, hydroxyl and nitro;

R⁹ is selected from the group consisting of lower alkyl, lower alkenyl, lower alkynyl, aralkyl, alkoxyalkyl, lower aminoalkyl, arylaminocarbonylalkyl, aminocarbonylalkyl, arylaminocarbonylalkyl, arylcarbonylalkyl, alkylthioalkyl, arylsulfonylaminoalkyl, cycloalkyl, cycloalkylalkyl, cycloalkenylalkyl, cycloalkylthioalkyl, carbonylalkyl, carbonylheterocyclylcarbonylalkyl, haloalkyl, heterocycloalkyl and hydroxyalkyl, any of which may be optionally substituted with one or more radicals independently selected from alkoxy, lower alkylacyl, amino, lower aminoalkyl, lower alkyl, cyano, halo, haloalkyl, hydroxy and nitro;

R¹⁰ is selected from the group consisting of lower alkyl and hydrogen, or R⁹ and R¹⁰ may combine to form an optionally substituted heterocycloalkyl or heteroaryl;

Ar is selected from the group consisting of phenyl, 2-pyridyl, or 2,6 pyrimidinyl, any of which may be optionally substituted with one or more radicals independently selected from lower alkylacyl, lower alkyl, lower alkenyl, lower alkynyl, lower alkoxy, alkoxyalkyl, amino, lower aminoalkyl, lower alkylaminocarbonyl, lower alkylcarbonylamino, carboxy, halo, lower haloalkyl, hydrogen, hydroxyalkyl and hydroxy, any of which may be optionally substituted with one or more radicals independently selected from lower alkyl, alkoxy, lower alkylacyl, amino, lower aminoalkyl, cyano, halo, haloalkyl, hydroxyl and nitro;

L, T, X and Y are each independently selected from the group consisting of N, C, O and S;

M is selected from the group consisting of N, C and S;

Q, U, V, W are each independently selected from the group consisting of N and C;

Z is selected from the group consisting of N, C(O), C and O;

wherein V, W, X, Y and Z taken together form an unsaturated ring containing at least one carbon atom;

with the proviso that when V and W are carbon, then X, Y, and Z are not all nitrogen;

with the proviso that when VWXZY does not form an aromatic ring, then Z must be C(O);

with the proviso that when Y is oxygen, then R⁷ is null and Ar is not an un-substituted phenyl or a chloro monosubstituted phenyl; and

With the proviso that when Z is C, then R⁷ is not hydrogen.

In a broad aspect, the subject invention provides for novel compounds, pharmaceutical compositions and methods of making and using the compounds and compositions. These compounds possess useful p38 kinase inhibiting or modulating activity, and may be used in the treatment or prophylaxis of a disease or condition in which the activity or hyperactivity of p38 kinase forms a contributory part. These compounds can inhibit and/or modulate the activity of p38 kinase.

### DETAILED DESCRIPTION OF THE INVENTION

In certain embodiments, the compounds of the present invention have structural Formula II:

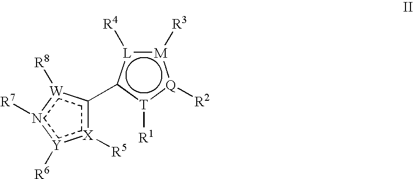

wherein:

R¹ is selected from the group consisting of lower alkyl, lower acylalkyl, lower alkoxy, amide, amino, lower aminoalkyl, lower alkylether, halo, hydrogen, hydroxy, hydroxyalkyl and null, any of which may be optionally substituted;

R² is selected from the group consisting of —C(O)R⁹, —C[N(OR¹²)]R¹³ and —S(O)ₙR¹⁵;

n is 0, 1 or 2:

R³ is selected from the group consisting of lower alkyl, lower aminoalkyl, halo, lower haloalkyl, hydrogen, hydroxy and null, any of which may be optionally substituted;

R⁴ is selected from the group consisting of lower alkyl, halo, hydrogen and null, any of which may be optionally substituted;

R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null, O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;

R⁷ is selected from the group consisting of acyl, lower alkyl, lower alkylether, hydrogen, hydroxy, hydroxyalkyl and null, any of which may be optionally substituted;

R⁸ is selected from the group consisting of aryl and heteroaryl, either of which may be optionally substituted;

R⁹ is selected from the group consisting of NR¹⁶R¹⁷, OR¹⁸, SR¹⁹, lower alkyl, lower alkenyl, lower alkynyl, lower aminoalkyl, aralkyl, aryl, arylamino, arylcarbonyl, lower carbonylalkyl, heteroaralkyl, hydrogen and thioalkyl, any of which may be optionally substituted;

R¹⁰, R¹¹, R¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted;

R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;

R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and

R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

The invention further provides for compounds of Formula III wherein:

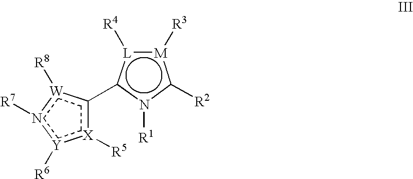

wherein:

R¹ is selected from the group consisting of lower alkoxy, lower alkyl, halo, hydrogen, hydroxy and null, any of which may be optionally substituted;

R² is selected from the group consisting of —C(O)R⁹ and —S(O)ₙR¹⁵;

n is 0, 1 or 2;

R³ is selected from the group consisting of lower alkoxy, lower alkyl, halo, hydrogen, hydroxy and null, any of which may be optionally substituted;

R⁴ is selected from the group consisting of lower alkyl, halo, haloalkyl, hydrogen and null, any of which may be optionally substituted;

R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null, O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;

R⁷ is selected from the group consisting of lower acyl, lower alkyl, halo, hydrogen, hydroxyl and null, any of which may be optionally substituted;

R⁸ is selected from the group consisting of aryl and heteroaryl, either of which may be optionally substituted;

R⁹ is selected from the group consisting of NR¹⁶R¹⁷, OR¹⁸, SR¹⁹, lower alkyl, lower alkenyl, alkynyl, amino, lower aminoalkyl, aralkyl, aryl, arylamino, arylcarbonyl, arylthio, arylsulfonyl, carbonylalkyl, carboxy, cycloalkyl, cycloalkylalkyl, cycloalkenyl, cycloalkylamino, haloalkyl, heteroaralkyl, heterocycloalkyl, heterocycloalkylalkyl, hydrogen, hydroxyalkyl, O-carbamoyl and N-carbamoyl, any of which may be optionally substituted;

R¹⁰, R¹¹, R¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted,

R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;

R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and

R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

In further embodiments the invention provides for compounds of Formula VII:

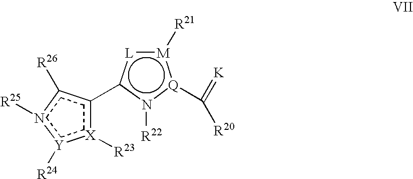

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention further provides for compounds having structural Formula VIII:

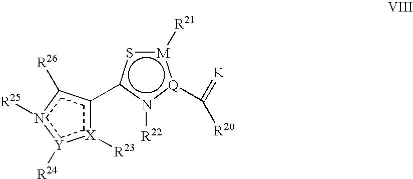

wherein:

K is selected from the group consisting of O, S and NR²⁷:

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention provides for compounds having structural Formula IX:

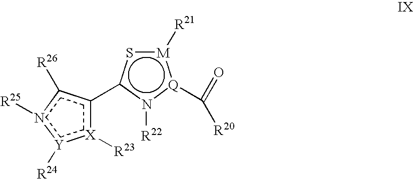

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention provides for compounds having structural Formula X:

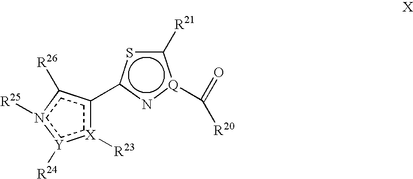

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted:

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention provides for compounds having structural Formula XI:

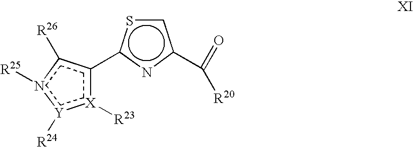

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention provides for compounds of Formula XI wherein R²⁶ is optionally substituted phenyl.

The invention provides for compounds of Formula XI wherein R²³ or R²⁴ is optionally substituted alkyl, alkoxyalkyl, aminoalkyl, heterocycloalkyl, hydrogen or null.

The invention provides for compounds of Formula XI wherein R²⁰ is optionally substituted amine, alkylamine, heteroarylalkyl or OR³².

The invention yet further provides for compounds having structural Formula XII:

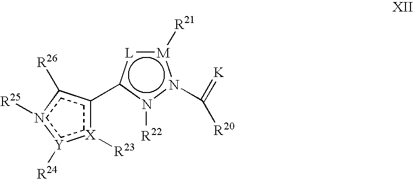

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²¹ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

In further embodiments the invention provides for compounds having structural Formula XIII:

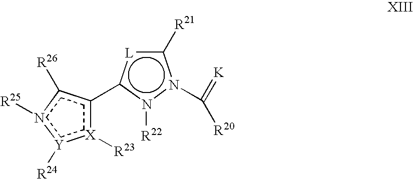

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention further provides for compounds having structural Formula XIV:

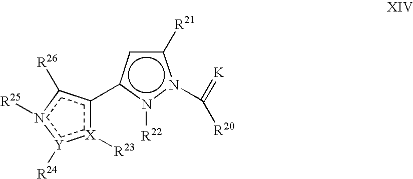

wherein:

K is selected from the group consisting of O, S and NR²⁷;

L is selected from the group consisting of CR²⁸, NR²⁹, S and N;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention provides for compounds having structural Formula XV:

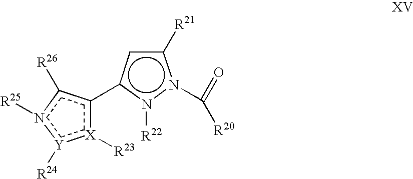

wherein:

K is selected from the group consisting of O, S and NR²⁷;

L is selected from the group consisting of CR²⁸, NR²⁹, S and O;

Y and X are each independently selected from the group consisting of N, C, O and S;

M is selected from the group consisting of C, O and S;

Q is selected from the group consisting of C, N and S;

R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;

R²¹ is selected from the group consisting of acyl, acylalkyl, alkoxy, alkoxyalkyl, alkyl, amide, amino, aminoalkyl, hydrogen, hydroxy and null, any of which may be optionally substituted;

R²² is selected from the group consisting of alkoxy, alkyl, ether, halo, lower haloalkyl, amino, hydroxyl, lower aminoalkyl, halo, hydrogen and null, any of which may be optionally substituted;

R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;

R²⁵ is selected from the group consisting of acyl, alkyl, carboxyalkyl, ether, halo, hydrogen, hydroxy, hydroxyalkyl and null, any of which may be optionally substituted;

R²⁶ is selected from the group consisting of aryl and heteroaryl, either of which may be optionally substituted;

R²⁷ is selected from the group consisting of alkoxy, alkyl, halo and hydrogen, any of which may be optionally substituted;

R²⁸ is selected from the group consisting of alkyl, alkoxy, alkynyl, halo, haloalkyl and hydrogen, any of which may be optionally substituted;

R²⁹ is selected from the group consisting of alkoxy, alkyl, amino, hydrogen and hydroxy, any of which may be optionally substituted;

R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl and heterocycloalkyl and thioalkyl any of which may be optionally substituted;

R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and

R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

The invention provides for compounds of Formula XV wherein R²⁶ is optionally substituted phenyl.

The invention provides for compounds of Formula XV wherein R²³ or R²⁴ is optionally substituted alkyl, heterocycloalkyl, hydrogen or null.

The invention provides for compounds of Formula XV wherein R²⁰ is optionally substituted alkyl, alkylamine, cycloalkylalkyl, heteroarylalkyl or arylamine.

The invention provides for compounds of Formula I-XV for use in the inhibition of p38 kinase for the treatment of disease.

The invention provides for compounds of Formula I-XV administered in combination with another therapeutic agent.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. Examples of acyl groups include formyl, alkanoyl and aroyl radicals.

The term “acylamino” embraces an amino radical substituted with an acyl group. An example of an “acylamino” radical is acetylamino (CH₃C(O)NH—).

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkoxyalkoxy,” as used herein, alone or in combination, refers to one or more alkoxy groups attached to the parent molecular moiety through another alkoxy group. Examples include ethoxyethoxy, methoxypropoxyethoxy, ethoxypentoxyethoxyethoxy and the like.

The term “alkoxyalkyl,” as used herein, alone or in combination, refers to an alkoxy group attached to the parent molecular moiety through an alkyl group. The term “alkoxyalkyl” also embraces alkoxyalkyl groups having one or more alkoxy groups attached to the alkyl group, that is, to form monoalkoxyalkyl and dialkoxyalkyl groups.

The term “alkoxycarbonyl,” as used herein, alone or in combination, refers to an alkoxy group attached to the parent molecular moiety through a carbonyl group. Examples of such “alkoxycarbonyl” groups include methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an alkyl radical. More preferred alkoxycarbonylalkyl radicals are “lower alkoxycarbonylalkyl” having lower alkoxycarbonyl radicals as defined above attached to one to six carbon atoms. Examples of such lower alkoxycarbonylalkyl radicals include methoxycarbonylmethyl.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an amino group attached to the parent molecular moiety through an alkyl group.

The term “alkylaminocarbonyl” as used herein, alone or in combination, refers to an alkylamino group attached to the parent molecular moiety through a carbonyl group. Examples of such radicals include N-methylaminocarbonyl and N,N-dimethylcarbonyl.

The term “alkylcarbonyl” and “alkanoyl,” as used herein, alone or in combination, refers to an alkyl group attached to the parent molecular moiety through a carbonyl group. Examples of such groups include methylcarbonyl and ethylcarbonyl.

The term “alkylidene,” as used herein, alone or in combination, refers to an alkenyl group in which one carbon atom of the carbon-carbon double bond belongs to the moiety to which the alkenyl group is attached.

The term “alkylsulfinyl,” as used herein, alone or in combination, refers to an alkyl group attached to the parent molecular moiety through a sulfinyl group. Examples of alkylsulfinyl groups include methylsulfinyl, ethylsulfinyl, butylsulfinyl and hexylsulfinyl.

The term “alkylsulfonyl,” as used herein, alone or in combination, refers to an alkyl group attached to the parent molecular moiety through a sulfonyl group. Examples of alkylsulfinyl groups include methanesulfonyl, ethanesulfonyl, tert-butanesulfonyl, and the like.

The term “alkylthio,” as used herein, alone or in combination, refers to an alkyl thioether (R—S—) radical wherein the term alkyl is as defined above. Examples of suitable alkyl thioether radicals include methylthio, ethylthio, n-propylthio, isopropylthio, n-butylthio, iso-butylthio, sec-butylthio, tert-butylthio, ethoxyethylthio, methoxypropoxyethylthio, ethoxypentoxyethoxyethylthio and the like.

The term “alkylthioalkyl” embraces alkylthio radicals attached to an alkyl radical. Alkylthioalkyl radicals include “lower alkylthioalkyl” radicals having alkyl radicals of one to six carbon atoms and an alkylthio radical as described above. Examples of such radicals include methylthiomethyl.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl, and the like.

The term “amido,” as used herein, alone or in combination, refers to an amino group as described below attached to the parent molecular moiety through a carbonyl group. The term “C-amido” as used herein, alone or in combination, refers to a —C(═O)—NR₂ group with R as defined herein. The term “N-amido” as used herein, alone or in combination, refers to a RC(═O)NH— group, with R as defined herein.

The term “amino,” as used herein, alone or in combination, refers to —NRR′, wherein R and R′ are independently selected from the group consisting of hydrogen, alkenyl, alkoxy, alkoxyalkyl, alkoxycarbonyl, alkyl, alkylcarbonyl, aryl, arylalkenyl, arylalkyl, cycloalkyl, haloalkylcarbonyl, heteroaryl, heteroarylalkenyl, heteroarylalkyl, heterocycle, heterocycloalkenyl, and heterocycloalkyl, wherein the aryl, the aryl part of the arylalkenyl, the arylalkyl, the heteroaryl, the heteroaryl part of the heteroarylalkenyl and the heteroarylalkyl, the heterocycle, and the heterocycle part of the heterocycloalkenyl and the heterocycloalkyl can be optionally substituted as defined herein with one, two, three, four, or five substituents.

The term “aminoalkyl,” as used herein, alone or in combination, refers to an amino group attached to the parent molecular moiety through an alkyl group. Examples include aminomethyl, aminoethyl and aminobutyl. The term “alkylamino” denotes amino groups which have been substituted with one or two alkyl radicals. Suitable “alkylamino” groups may be mono- or dialkylated, forming groups such as, for example, N-methylamino, N-ethylamino, N,N-dimethylamino, N,N-diethylamino and the like.

The terms “aminocarbonyl” and “carbamoyl,” as used herein, alone or in combination, refer to an amino-substituted carbonyl group, wherein the amino group can be a primary or secondary amino group containing substituents selected from alkyl, aryl, aralkyl, cycloalkyl, cycloalkylalkyl radicals and the like.

The term “aminocarbonylalkyl,” as used herein, alone or in combination, refers to an aminocarbonyl radical attached to an alkyl radical, as described above. An example of such radicals is aminocarbonylmethyl. The term “amidino” denotes an —C(NH)NH₂ radical. The term “cyanoamidino” denotes an —C(N—CN)NH₂ radical.

The term “aralkenyl” or “arylalkenyl,” as used herein, alone or in combination, refers to an aryl group attached to the parent molecular moiety through an alkenyl group.

The term “aralkoxy” or “arylalkoxy,” as used herein, alone or in combination, refers to an aryl group attached to the parent molecular moiety through an alkoxy group.

The term “aralkyl” or “arylalkyl,” as used herein, alone or in combination, refers to an aryl group attached to the parent molecular moiety through an alkyl group.

The term “aralkylamino” or “arylalkylamino,” as used herein, alone or in combination, refers to an arylalkyl group attached to the parent molecular moiety through a nitrogen atom, wherein the nitrogen atom is substituted with hydrogen.

The term “aralkylidene” or “arylalkylidene,” as used herein, alone or in combination, refers to an aryl group attached to the parent molecular moiety through an alkylidene group

The term “aralkylthio” or “arylalkylthio,” as used herein, alone or in combination, refers to an arylalkyl group attached to the parent molecular moiety through a sulfur atom.

The term “aralkynyl” or “arylalkynyl,” as used herein, alone or in combination, refers to an aryl group attached to the parent molecular moiety through an alkynyl group.

The term “aralkoxycarbonyl,” as used herein, alone or in combination, refers to a radical of the formula aralkyl-O—C(O)— in which the term “aralkyl,” has the significance given above. Examples of an aralkoxycarbonyl radical are benzyloxycarbonyl (Z or Cbz) and 4-methoxyphenylmethoxycarbonyl (MOS).

The term “aralkanoyl,” as used herein, alone or in combination, refers to an acyl radical derived from an aryl-substituted alkanecarboxylic acid such as benzoyl, phenylacetyl, 3-phenylpropionyl (hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like. The term “aroyl” refers to an acyl radical derived from an arylcarboxylic acid, “aryl” having the meaning given below. Examples of such aroyl radicals include substituted and unsubstituted benzoyl or napthoyl such as benzoyl, 4-chlorobenzoyl, 4-carboxybenzoyl, 4-(benzyloxycarbonyl)benzoyl, 1-naphthoyl, 2-naphthoyl, 6-carboxy-2-naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylamino” as used herein, alone or in combination, refers to an aryl group attached to the parent moiety through an amino group, such as methylamino, N-phenylamino, and the like.

The terms “arylcarbonyl” and “aroyl,” as used herein, alone or in combination, refer to an aryl group attached to the parent molecular moiety through a carbonyl group.

The term “aryloxy,” as used herein, alone or in combination, refers to an aryl group attached to the parent molecular moiety through an oxygen atom.

The term “arylsulfonyl,” as used herein, alone or in combination, refers to an aryl group attached to the parent molecular moiety through a sulfonyl group.

The term “arylthio,” as used herein, alone or in combination, refers to an aryl group attached to the parent molecular moiety through a sulfur atom.

The terms “carboxy” or “carboxyl”, whether used alone or with other terms, such as “carboxyalkyl”, denotes —CO₂H.

The terms “benzo” and “benz,” as used herein, alone or in combination, refer to the divalent radical C₆H₄═ derived from benzene. Examples include benzothiophene and benzimidazole.

The term “O-carbamyl” as used herein, alone or in combination, refers to a —OC(O)NR, group-with R as defined herein.

The term “N-carbamyl” as used herein, alone or in combination, refers to a ROC(O)NH— group, with R as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydonapthalene, octahydronapthalene as well as the multicyclic (multicentered) saturated or partially unsaturated type. The latter type of isomer is exemplified in general by bicyclo[2,2,2]octane, bicyclo[2,2,2]octane, bicyclo[1,1,1]pentane, camphor and bicyclo[3,2,1]octane.

The term “ester,” as used herein, alone or in combination, refers to a carbonyl group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a halohydrocarbyl group attached at two or more positions. Examples include fluoromethylene (—CFH—), difluoromethylene (—CF₂—), chloromethylene (—CHCl—) and the like. Examples of such haloalkyl radicals include chloromethyl, 1-bromoethyl, fluoromethyl, difluoromethyl, trifluoromethyl, 1,1,1-trifluoroethyl, perfluorodecyl and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH2-NH—OCH3.

The term “heteroaryl,” as used herein, alone or in combination, refers to 3 to 7 membered, preferably 5 to 7 membered, unsaturated heterocyclic rings wherein at least one atom is selected from the group consisting of O, S, and N. Heteroaryl groups are exemplified by: unsaturated 3 to 7 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl [e.g., 4H-1,2,4-triazolyl, 1H-1,2,3-triazolyl, 2H-1,2,3-triazolyl, etc.]tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic groups containing an oxygen atom, for example, pyranyl, furyl, etc.; unsaturated 3 to 6-membered heteromonocyclic groups containing a sulfur atom, for example, thienyl, etc.; unsaturated 3- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.]etc.; unsaturated condensed heterocyclic groups containing 1 to 2 oxygen atoms and 1 to 3 nitrogen atoms [e.g. benzoxazolyl, benzoxadiazolyl, etc.]; unsaturated 3 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.]and isothiazolyl; unsaturated condensed heterocyclic groups containing 1 to 2 sulfur atoms and 1 to 3 nitrogen atoms [e.g., benzothiazolyl, benzothiadiazolyl, etc.]and the like. The term also embraces radicals where heterocyclic radicals are fused with aryl radicals. Examples of such fused bicyclic radicals include benzofuryl, benzothienyl, and the like.

The term “heteroaralkenyl” or “heteroarylalkenyl,” as used herein, alone or in combination, refers to a heteroaryl group attached to the parent molecular moiety through an alkenyl group.

The term “heteroaralkoxy” or “heteroarylaIkoxy,” as used herein, alone or in combination, refers to a heteroaryl group attached to the parent molecular moiety through an alkoxy group.

The term “heteroaralkyl” or “heteroarylalkyl,” as used herein, alone or in combination, refers to a heteroaryl group attached to the parent molecular moiety through an alkyl group.

The term “heteroaralkylidene” or “heteroarylalkylidene,” as used herein, alone or in combination, refers to a heteroaryl group attached to the parent molecular moiety through an alkylidene group.

The term “heteroaryloxy,” as used herein, alone or in combination, refers to a heteroaryl group attached to the parent molecular moiety through an oxygen atom.

The term “heteroarylsulfonyl,” as used herein, alone or in combination, refers to a heteroaryl group attached to the parent molecular moiety through a sulfonyl group.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides. N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “heterocycloalkenyl,” as used herein, alone or in combination, refers to a heterocycle group attached to the parent molecular moiety through an alkenyl group.

The term “heterocycloalkoxy,” as used herein, alone or in combination, refers to a heterocycle group attached to the parent molecular group through an oxygen atom.

The term “heterocycloalkyl,” as used herein, alone or in combination, refers to an alkyl radical as defined above in which at least one hydrogen atom is replaced by a heterocyclo radical as defined above, such as pyrrolidinylmethyl, tetrahydrothienylmethyl, pyridylmethyl and the like.

The term “heterocycloalkylidene,” as used herein, alone or in combination, refers to a heterocycle group attached to the parent molecular moiety through an alkylidene group.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl” as used herein, alone or in combination, refers to a linear or branched alkyl group having one to about ten carbon atoms any one of which may be substituted with one or more hydroxyl radicals. Examples of such radicals include hydroxymethyl, hydroxyethyl, hydroxypropyl, hydroxybutyl and hydroxyhexyl.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptoalkyl” as used herein, alone or in combination, refers to an R′SR— group, where R and R′ are as defined herein.

The term “mercaptomercaptyl” as used herein, alone or in combination, refers to a RSR′S— group, where R is as defined herein.

The term “mercaptyl” as used herein, alone or in combination, refers to an RS— group, where R is as defined herein.

The term “null” refers to a lone electron pair.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “oxo” as used herein, alone or in combination, refers to a doubly bonded oxygen.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S and —S—.

The term “sulfinyl,” as used herein, alone or in combination, refers to —S(O)—.

The term “sulfonyl,” as used herein, alone or in combination, refers to —SO₂—.

The term “N-sulfonamido” refers to a RS(═O)₂NH— group with R as defined herein.

The term “S-sulfonamido” refers to a —S(═O)₂NR₂, group, with R as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thioether,” as used herein, alone or in combination, refers to a thio group bridging two moieties linked at carbon atoms.

The term “thiol,” as used herein, alone or in combination, refers to an —SH group.

The term “thiocarbonyl,” as used herein, when alone includes thioformyl —C(S)H and in combination is a —C(S) group.

The term “N-thiocarbamyl” refers to an ROC(S)NH— group, with R as defined herein.

The term “O-thiocarbamyl” refers to a —OC(S)NR, group with R as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, NHCH₃, N(CH₃)₂, SH, SCH₃, C(O)CH₃, CO₂CH₃, CO₂H, C(O)NH₂, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH2CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particuar moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of alkyl, cycloalkyl, heteroalkyl, aryl, heteroaryl and heterocycloalkyl. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence.

The term “bond” refers to a covalent linkage between two atoms, or two moieties when the atoms joined by the bond are considered to be part of larger substructure. A bond may be single, double, or triple unless otherwise specified.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“p38 kinase inhibitor” is used herein to refer to a compound that exhibits an IC₅₀ with respect to p38 kinase activity of no more than about 100 μM and more typically not more than about 50 μM, as measured in the p38α Assay described generally hereinbelow. “IC₅₀” is that concentration of inhibitor which reduces the activity of an enzyme (e.g., p38 kinase) to half-maximal level. Representative compounds of the present invention have been discovered to exhibit inhibitory activity against p38 kinase. Compounds of the present invention preferably exhibit an IC₅₀ with respect to p38 kinase of no more than about 10 μM, more preferably, no more than about 5 μM, even more preferably not more than about 1 μM, and most preferably, not more than about 200 nM, as measured in the p38 kinase assay(s) described herein.

The phrase “therapeutically effective” is intended to qualify the amount of active ingredients used in the treatment of a disease or disorder. This amount will achieve the goal of reducing or eliminating the said disease or disorder.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended uses.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. For a more complete discussion of the preparation and selection of salts, refer to Pharmaceutical Salts: Properties, Selection, and Use (Stahl, P. Heinrich. Wiley-VCHA, Zurich, Switzerland, 2002).

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, milling, neutralization, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. The formulation may have ingredients, such as lubricants that facilitate how it operates within a dispensing device.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described. The formulation may also be presented as a frozen bag or in a ready to use admixture.

Formulations for parenteral or ophthalmic administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation including coatings that may be applied to an implantable device such as a stent. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments, sprays or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Gels for topical or transdermal administration of compounds of the subject invention may comprise, generally, a mixture of volatile solvents, nonvolatile solvents, and water. The volatile solvent component of the buffered solvent system may preferably include lower (C1-C6) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compounds of the invention with agents found in the following pharmacotherapeutic classifications as indicated below. These lists should not be construed to be closed, but should instead serve as illustrative examples common to the relevant therapeutic area at present. Moreover, combination regimens may include a variety of routes of administration and should include intravenous, intraocular, subcutaneous, dermal, inhaled topical, oral.

For the treatment of inflammatory pain, compounds according to the present invention may be administered with an agent selected from the group comprising: a) corticosteroids including betamethasone dipropionate (augmented and nonaugemnted), betamethasone valerate, clobetasol propionate, prednisone, methyl prednisolone, diflorasone diacetate, halobetasol propionate, anicinonide, dexamethasone, dexosimethasone, fluocinolone acetononide, fluocinonide, halocinonide, clocortalone pivalate, dexosimetasone, and flurandrenalide; b) non-steroidal anti-inflammatory drugs including salicylates, ibuprofen, ketoprofen, etodolac, diclofenac, meclofenamate sodium, naproxen, piroxicam, and celecoxib; c) muscle relaxants and combinations thereof with other agents, including cyclobenzaprine, baclofen, cyclobenzaprine/lidocaine, baclofen/cyclobenzaprine, and cyclobenzaprine/lidocaine/ketoprofen; d) anaesthetics and combinations thereof with other agents, including lidocaine, lidocaine/deoxy-D-glucose (an antiviral), prilocalne, and EMLA Cream [Eutectic Mixture of Local Anesthetics (lidocaine 2.5% and prilocalne 2.5%; an emulsion in which the oil phase is a eutectic mixture of lidocaine and prilocalne in a ratio of 1:1 by weight. This eutectic mixture has a melting point below room temperature and therefore both local anesthetics exist as a liquid oil rather then as crystals)]; i) opioids including codeine, loperamide, tramadol, morphine, fentanyl, oxycodone, hydrocodone, levorphanol, and butorphanol; j) topical counter-irritants including menthol, oil of wintergreen, camphor, eucalyptus oil and turpentine oil; k) topical cannabinoids including selective and non-selective CB1/CB2 ligands; 1) agents with analgesic and antipyretic properties including acetaminophen; m) agents that modify inflammatory mediators including infliximab; n) nitric oxide synthase inhibitors, particularly inhibitors of inducible nitric oxide stnthase; and other agents, such as capsaicin.

For the treatment of autoimmune disorders, compounds according to the present invention may be administered with an agent selected from the group comprising: corticosteroids including dexamethasome, prednisone, and methylprednisolone; immunosuppressant agents including azathioprine, cyclosporine, and immunoglobulins; and prostaglandin analogs including latanoprost, travoprost, bimatoprost, and unoprostone; prostaglandin analogs that modify inflammatory mediators including infliximab and rutuximab; and antimetabolites including methotrexate.

For the treatment of respiratory disorders, compounds according to the present invention may be administered with an agent selected from the group comprising: sympathomimetic agents including salmeterol, albuterol, terbutaline, metaproterenol, and ipratropium bromide; and mast cell stabilizers including cromolyn.

For the treatment of endocrine disorders, compounds according to the present invention may be administered with an agent selected from the group comprising: insulin and insulin derivatives: sulfonylureas agents including glimepiride and glipizide; biguanide agents including metformin; and PPAR modulators such as thiazolidnedione agents including pioglitazone and rosigliatzone.

For the treatment of oncologic diseases, proliferative disorders, and cancers, compounds according to the present invention may be administered with an agent selected from the group comprising: aromatase inhibitors, antiestrogen, anti-androgen, or gonadorelin agonists, topoisomerase 1 and 2 inhibitors, microtubule active agents, alkylating agents, antineoplastic antimetabolites, or platin containing compounds, lipid or protein kinase targeting agents, protein or lipid phosphatase targeting agents, anti-angiogentic agents, agents that induce cell differentiation, bradykinin I receptor antagonists, angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, inhibitors of Ras isoforms, telomerase inhibitors, protease inhibitors, metalloproteinase inhibitors, and aminopeptidase inhibitors.

For the treatment of ophthalmologic disorders and diseases of the eye, compounds according to the present invention may be administered with an agent selected from the group comprising: beta-blockers including timolol, betaxolol, levobetaxolol, carteolol, levobunolol, and propranolol; carbonic anhydrase inhibitors including brinzolamide and dorzolamide; α- and β-adrenergic antagonists including α1-adrenergic antagonists such as nipradilol and α2 agonists such as iopidine and brimonidine; miotics including pilocarpine and epinephrine; prostaglandin analogs including latanoprost, travoprost, bimatoprost, and unoprostone; corticosteroids including dexamethasone, prednisone, and methylprednisolone; and immunosuppressant agents including azathioprine, cyclosporine, and immunoglobulins.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating p38 kinase mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of p38 kinase mediated disorders.

Diseases or disorders in which p38 kinase plays a role, either directly or via pro-inflammatory cytokines including the cytokines TNF, IL-1, IL-6 and IL-8, include, without limitation: neurological diseases, autoimmune diseases, inflammatory diseases, bone-destructive disorders, proliferative disorders, neurodegenerative disorders, viral diseases, allergies, infectious diseases, heart attacks and other cardiovascular conditions, angiogenic disorders, reperfusion/ischemia in stroke, vascular hyperplasia, organ hypoxia, cardiac hypertrophy, thrombin-induced platelet aggregation, and conditions associated with prostaglandin endoperoxidase synthetase-2 (COX-2). The invention further extends to the particular disease of inflammatory pain.

Neurological diseases that may be prevented or treated to include, without limitation: Alzheimer's disease (AD), Parkinson's disease (PD), neuropathic pain including lower back pain, peripheral neuropathy, diabetic neuropathy, and multiple sclerosis.

Autoimmune diseases which may be prevented or treated include, without limitation: osteoarthritis, spondyloarthropathies, systemic lupus nephritis, rheumatoid arthritis, inflammatory bowel disease, ulcerative colitis, Crohn's disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs. host disease, or psoriasis. The invention further extends to the particular autoimmune disease rheumatoid arthritis.

Inflammatory diseases which may be prevented or treated include, without limitation: asthma, allergies, respiratory distress syndrome or acute or chronic pancreatitis. Furthermore, respiratory system diseases may be prevented or treated including but not limited to chronic obstructive pulmonary disease, and pulmonary fibrosis.

In addition, p38 inhibitors of this invention also exhibit inhibition of expression of inducible pro-inflammatory proteins such as prostaglandin endoperoxidase synthetase-2, otherwise known as cyclooxygenase-2 (COX-2) and are therefore of use in therapy. Pro-inflammatory mediators of the cyclooxygenase pathway derived from arachidonic acid, such as prostaglandins, are produced by inducible COX-2 enzyme. Regulation of COX-2 would regulate these pro-inflammatory mediators, which affect a wide variety of cells and are important and critical inflammatory mediators of a wide variety of disease states and conditions. In particular, these inflammatory mediators have been implicated in pain, such as in the sensitization of pain receptors, and edema. Accordingly, additional p38 mediated conditions which may be prevented or treated include edema, analgesia, fever and pain such as neuromuscular pain, headache, dental pain, arthritis pain and pain caused by cancer.

Metabolic diseases which may be treated or prevented include, without limitation, metabolic syndrome, insulin resistance, and Type 1 and Type 2 diabetes.

Dermatologic diseases including, without limitation, psoriasis and persistent itch, and other diseases related to skin and skin structure, may be treated or prevented with p38 inhibitors of this invention.

Ophthalmologic dieases which may be treated or prevented include, without limitation, dry eye (including Sjögren's syndrome), macular degeneration, closed and wide angle glaucoma, inflammation, and pain of the eye.

Hematological and non-hematological malignancies which may be treated or prevented include but are not limited to multiple myeloma, acute and chronic leukemias including Acute Lymphocytic Leukemia (ALL), Chronic Lymphocytic Leukemia (CLL), and Chronic Myelogenous Leukemia (CLL), lymphomas, including Hodgkin's lymphoma and non-Hodgkin's lymphoma (low, intermediate, and high grade), malignancies of the brain, head and neck, breast, lung, reproductive tract, upper digestive tract, pancreas, liver, renal, bladder, prostate and colorectal.

As a result of their p38 inhibitory activity, compounds of the invention have utility in the prevention and treatment of diseases associated with cytokine production including but not limited to those diseases associated with TNF, IL-1, IL-6 and IL-8 production.

The present invention includes compounds listed above in the form of salts, in particular acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein. The contents of United States prov appl'n No. 60/674,047 filed on Apr. 22, 2005 are hereby incorp'd by ref in their entirety.

### General Synthetic Methods for Preparing Compounds

Molecular embodiments of the present invention can be synthesized using standard synthetic techniques known to those of skill in the art. Schemes I-IV illustrate the general synthesis of intermediates of the present invention.

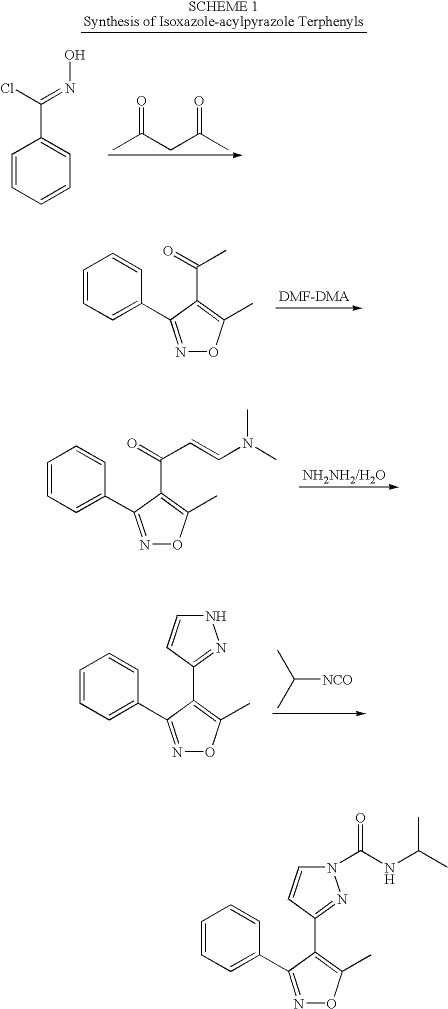

### EXAMPLE 1

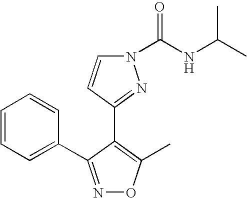

### 2-|3-(4-Fluoro-phenyl)-5-methyl-isoxazol-4-yl|-thiazole-4-carboxylic acid isopropylamide

Step 1

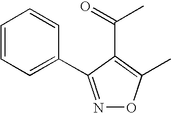

### 1-(5-Methyl-3-phenyl-isoxazol-4-yl)-ethanone

a-chlorobenzoyl oxime (15.5g, 0.1 mol, prepared as described in Journal of Heterocyclic Chemistry (2000), 37(6), 1505-1510) was dissolved in absolute EtOH (50 mL). Then acetylacetone (15 g, 0.15 mol) and triethylamine (15.2 g, 0.15 mol) were added. The resulting mixture was stirred overnight at 50° C. Reaction progress was monitored by TLC (EtOAc/Petroleum ether=1:5). Work-up: the mixture was concentrated, dissolved in EtOAc, washed with brine, dried with Na₂SO_{4,} and concentrated to an oil. The oil was further purified by column chromatography on silica gel (EtOAc/Petroleum ether 1:50) to give a colorless crystal (13 g, 65%).
Step 2

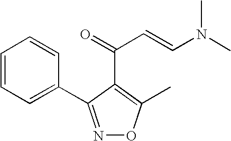

### 3-Dimethylamino-1-(5-methyl-3-phenyl-isoxazol-4-yl)-propenone

A 100 mL round bottom flask was charged with 1-(5-Methyl-3-phenyl-isoxazol-4-yl)-ethanone (4.02 g, 0.02 mol), and DMF-DMA (20 mL). The resulting mixture was refluxed overnight. Reaction progress was monitored by TLC (EtOAc/Petroleum ether=1/1). Work-up: the mixture was concentrated and purified by column chromatography (EtOAc/Petroleum ether=1/10) to afford a light yellow solid (3.4 g, 66%).
Step 3

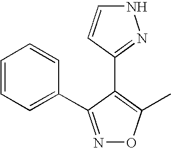

### 5-Methyl-3-phenyl-4-(1H-pyrazol-3-yl)-isoxazole

A 100 mL round bottom flask charged with hydrazine hydrate (6.3 g, 0.1 mol) at −20° C., treated with 3-Dimethylamino-1-(5-methyl-3-phenyl-isoxazol-4-yl)-propenone in EtOH (20 mL). The resulting mixture was stirred for 2 hours at this temperature, then warned to room temperature overnight, and stirred overnight. Reaction progress was monitored by TLC (EtOAc/Petroleum ether=2:1). Work-up: the mixture was concentrated and crystallized, giving a yellow solid (2.15 g, 96%).
Step 4

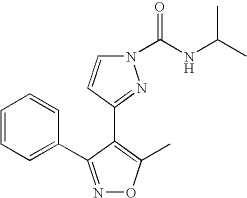

### 3-(5-Methyl-3-phenyl-isoxazol-4-yl)pyrazole-1-carboxylic acid isopropylamide

A 50 mL round bottom flask was charged with 5-methyl-3-phenyl-4-(pyrazolyl-5-)isoxazole (1.1 g, 5.0 mmol), DCM (10 mL), and isopropyl isocyanate (10 mmol). The mixture was stirred for 48 h at room temperature. Reaction progress was monitored by TLC (EtOAc/Petroleum ether=1/1). Work-up: the reaction mixture was concentrated and purified by column chromatography (EtOAc/Petroleum ether=1/10), to afford the product as white crystals (66.8% yield). ¹H NMR (400 MHz, CDCl₃) δ 8.19 (s, 1H), 7.56 (m, 2H), 7.45 (m, 3H), 6.15 (s, 1H), 4.15 (m, 1H), 2.66 (s, 3H), 1.33 (s, 3H), 1.28 (s, 3H).

### EXAMPLE 2

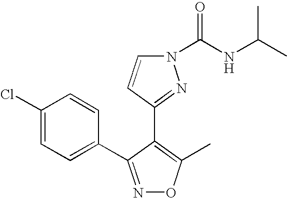

### 3-|3-(4-Chloro-phenyl)-5-methyl-isoxazol-4-yl|-pyrazole-1-carboxylic acid isopropylamide

The title compound was prepared analogously to 3-(5-Methyl-3-phenyl-isoxazol-4-yl)-pyrazole-1-carboxylic acid isopropylamide (Example 1), where a-chloro-4-chlorobenzoyl oxine was substituted for a-chlorobenzoyl oxime in step 1 of that sequence. ¹H NMR (400 MHz, CDCl₃) δ 8.20 (s, 1H), 7.50 (m, 2H), 7.38 (m, 2H), 6.76 (m, 1H), 6.17 (s, 1H), 4.12 (septet, 1H), 2.62 (s, 3H), 1.28 (d, 6H).

### EXAMPLE 3

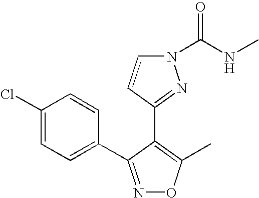

### 3-|3-(4-Chloro-phenyl)-5-methyl-isoxazol-4-yl|-pyrazole-1-carboxylic acid methylamide

The title compound was prepared analogously to 3-[3-(4-Chloro-phenyl)-5-methyl-isoxazol-4-yl]-pyrazole-1-carboxylic acid isopropylamide (Example 1), where methyl isocyanate was substituted for isopropyl isocyanate in the final step of that sequence. ¹H NMR (400 MHz, CDCl₃) δ 8.21 (s, 1H), 7.47 (d, 2H), 7.37 (d, 2H), 6.97 (bs, 1H), 6.13 (s, 1H), 3.03 (d, 3H), 2.60 (s, 3H).

### EXAMPLE 4

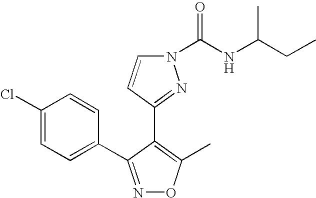

### 3-|3-(4-Chloro-phenyl)-5-methyl-isoxazol-4-yl|-pyrazole-1-carboxylic acid sec-butylamide

A 25 mL round bottom flask was charged with 5-Methyl-3-(4-chlorophenyl)-4-(1H-pyrazol-3-yl)-isoxazole (0.5 g, 1.93 mmol, prepared analogously to 5-Methyl-3-phenyl-4-(1H-pyrazol-3-yl)-isoxazole, described in step 3 of Example 1), Et₃N (0.3 g, 2.97 mmol), and DCM (10 mL). The resulting solution was treated with triphosgene in DCM (0.3 g, 11.0 mL), while at 0° C., then stirred for 3 hours at room temperature. This solution was treated with isobutylamine in DCM (0.44 g, 6 mmol in 6 mL) at 0° C., then stirred for 2 h at room temperature. Work-up: the mixture washed with brine, dried with MgSO₄, and concentrated. The crude material was purified by column chromatography (60% Ethyl Acetate/Petroleum ether) giving the product as a white solid (350 mg, 51%). ¹H NMR (400 MHz, CDCl₃) δ 8.19 (d, 1H), 7.49 (d, 2H), 7.38 (d, 2H), 6.75 (m, 1H), 6.17 (d, 1H), 3.91 (m, 1H), 2.62 (s, 3H), 1.59 (m, 3H), 1.25 (d, 3H), 0.95 (t, 3H).

### EXAMPLE 5

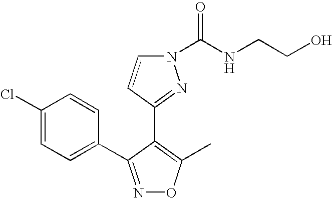

### 3-|3-(4-Chloro-phenyl)-5-methyl-isoxazol-4-yl|-pyrazole-1-carboxylic acid (2-hydroxy-ethyl)-amide

The title compound was prepared analogously to 3-[3-(4-Chloro-phenyl)-5-methyl-isoxazol-4-yl]-pyrazole-1-carboxylic acid sec-butylamide (Example 4), where EtOH amine was substituted for sec-butylamine in the final step of that sequence. ¹H NMR (400 MHz, CDCl₃) δ 8.21 (s, 1H), 7.50 (d, 2H), 7.41 (d, 2H), 6.16 (s, 1H), 3.86 (t, 2H), 3.61 (m, 2H), 2.63 (s, 3H).

### EXAMPLE 6

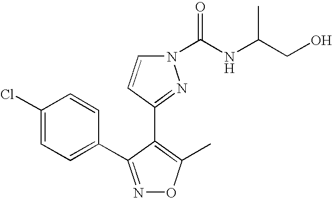

### 3-|3-(4-Chloro-phenyl)-5-methyl-isoxazol-4-yl|-pyrazole-1-carboxylic acid (2-hydroxy-1-methyl-ethyl)-amide

The title compound was prepared analogously to 3-[3-(4-Chloro-phenyl)-5-methyl-isoxazol-4-yl]-pyrazole-1-carboxylic acid sec-butylamide (Example 4), where 2-amino-propan-1-ol was substituted for sec-butylamine in the final step of that sequence. ¹H NMR (400 MHz, CDCl₃) δ 8.19 (d, 1H), 7.50 (d, 2H), 7.40 (d, 2H), 7.21 (m, 1H), 6.18 (d, 1H), 3.44 (m, 2H), 2.62 (s, 3H), 1.32 (d, 3H).

### EXAMPLE 7

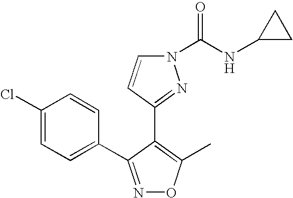

### 3-|3-(4-Chloro-phenyl)-5-methyl-isoxazol-4-yl|-pyrazole-1-carboxylic acid cyclopropylamide

The title compound was prepared analogously to 3-[3-(4-Chloro-phenyl)-5-methyl-isoxazol-4-yl]-pyrazole-1-carboxylic acid sec-butylamide (Example 4), where cyclopropyl amine was substituted for sec-butylamine in the final step of that sequence. ¹H NMR (400 MHz, CDCl₃) δ 8.21 (d, 1H), 7.49 (d, 2H), 7.38 (d, 2H), 7.02 (m, 1H), 6.15 (d, 1H), 2.80 (m, 1H), 2.60 (s, 3H), 0.90 (m, 2H), 0.68 (m, 2H).

### EXAMPLE 8

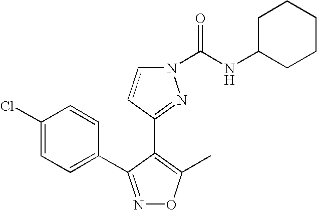

### 3-|3-(4-Chloro-phenyl)-5-methyl-isoxazol-4-yl|-pyrazole-1-carboxylic acid cyclohexylamide

The title compound was prepared analogously to 3-[3-(4-Chloro-phenyl)-5-methyl-isoxazol-4-yl]-pyrazole-1-carboxylic acid sec-butylamide (Example 4), where cyclohexyl amine was substituted for sec-butylamine in the final step of that sequence. ¹H NMR (400 MHz, CDCl₃) δ 8.20 (m, 1H), 7.49 (d, 2H), 7.38 (d, 2H), 6.82 (m, 1H), 6.18 (s, 1H), 2.61 (s, 3H), 1.99 (m, 2H), 1.74 (m, 2H), 1.62 (m, 2H), 1.43 (m, 2H), 1.26 (m, 2H).

### EXAMPLE 9

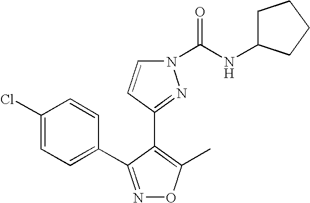

### 3-|3-(4-Chloro-phenyl)-5-methyl-isoxazol-4-yl|-pyrazole-1-carboxylic acid cyclopentylamide

The title compound was prepared analogously to 3-[3-(4-Chloro-phenyl)-5-methyl-isoxazol-4-yl]-pyrazole-1-carboxylic acid sec-butylamide (Example 4), where cyclopentyl amine was substituted for sec-butylamine in the final step of that sequence. ¹H NMR (400 MHz, CDCl₃) δ 8.20 (d, 1H), 7.47 (d, 2H), 7.38 (d, 2H), 6.83 (m, 1H), 6.17 (s, 1H), 4.24 (septet, 1H), 2.59 (s, 3H), 2.06 (m, 2H), 1.69 (m, 5H), 1.54 (m, 2H).

### EXAMPLE 10

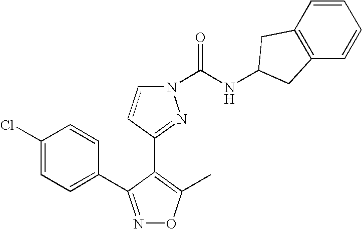

### 3-|3-(4-Chloro-phenyl)-5-methyl-isoxazol-4-yl|-pyrazole-1-carboxylic acid indan-2-ylamide

The title compound was prepared analogously to 3-[3-(4-Chloro-phenyl)-5-methyl-isoxazol-4-yl]-pyrazole-1-carboxylic acid sec-butylamide (Example 4), where 2-indanamine was substituted for sec-butylamine in the final step of that sequence. ¹H NMR (400 MHz, CDCl₃) δ 8.24 (d, 1H), 7.49 (d, 2H), 7.35 (d, 2H), 7.22-7.30 (m, 4H), 7.14 (m, 1H), 6.20 (d, 1H), 4.81 (m, 1H), 3.45 (m, 2H), 2.96 (m, 2H), 2.58 (s, 3H).

### EXAMPLE 11

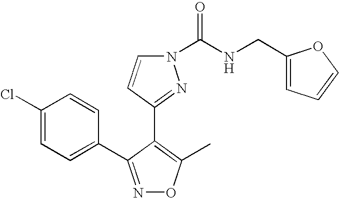

### 3-|3-(4-Chloro-phenyl)-5-methyl-isoxazol-4-yl|-pyrazole-1-carboxylic acid (furan-2-ylmethyl)-amide

The title compound was prepared analogously to 3-[3-(4-Chloro-phenyl)-5-methyl-isoxazol-4-yl]-pyrazole-1-carboxylic acid sec-butylamide (Example 4), where C-Furan-2-yl-methylamine was substituted for sec-butylamine in the final step of that sequence. ¹H NMR (400 MHz, CDCl₃) δ 8.23 (s, 1H), 7.49 (d, 2H), 7.42 (s, 1H), 7.37 (d, 2H), 6.37 (d, 1H), 6.32 (d, 1H), 4.62 (d, 2H), 2.62 (s, 3H).

### EXAMPLE 12

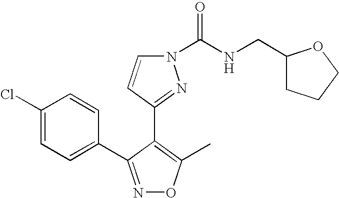

### 3-|3-(4-Chloro-phenyl)-5-methyl-isoxazol-4-yl|-pyrazole-1-carboxylic acid (tetrahydro-furan-2-ylmethyl)-amide

The title compound was prepared analogously to 3-[3-(4-Chloro-phenyl)-5-methyl-isoxazol-4-yl]-pyrazole-1-carboxylic acid sec-butylamide (Example 4), where C-(Tetrahydro-furan-2-yl)-methylamine was substituted for sec-butylamine in the final step of that sequence. ¹H NMR (400 MHz, CDCl₃) δ 8.19 (d, 1H), 7.50 (d, 2H), 7.39 (d, 2H), 7.28 (m, 1H), 6.15 (s, 1H), 4.09 (m, 1H), 3.87 (m, 1H), 3.79 (m, 1H), 3.64 (m, 1H), 3.40 (m, 1H), 2.63 (s, 3H), 2.04 (m, 1H), 1.93 (m, 2H), 1.61 (m, 2H).

### EXAMPLE 13

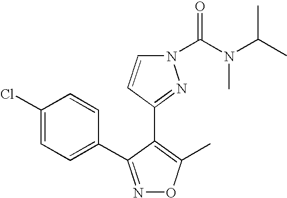

### 3-|3-(4-Chloro-phenyl)-5-methyl-isoxazol-4-yl|-pyrazole-1-carboxylic acid isopropyl-methyl-amide

The title compound was prepared analogously to 3-[3-(4-Chloro-phenyl)-5-methyl-isoxazol-4-yl]-pyrazole-1-carboxylic acid sec-butylamide (Example 4), where isopropyl-methyl-amine was substituted for sec-butylamine in the final step of that sequence. ¹H NMR (400 MHz, CDCl₃) δ 8.12 (s, 1H), 7.47 (d, 2H), 7.38 (d, 2H), 6.12 (s, 1H), 4.65 (septet, 1H), 2.99 (s, 3H), 2.60 (s, 3H), 1.21 (d, 6H).

### EXAMPLE 14

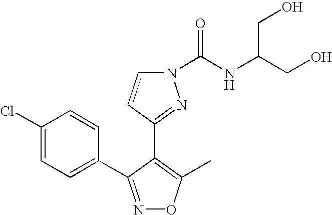

### 3-|3-(4-Chloro-phenyl)-5-methyl-isoxazol-4-yl|-pyrazole-1-carboxylic acid (2-hydroxy-1-hydroxymethyl-ethyl)-amide

The title compound was prepared analogously to 3-[3-(4-Chloro-phenyl)-5-methyl-isoxazol-4-yl]-pyrazole-1-carboxylic acid sec-butylamide (Example 4), where 2-Amino-propane-1,3-diol was substituted for sec-butylamine in the final step of that sequence. ¹H NMR (400 MHz, CDCl₃) δ 8.19 (s, 1H), 7.61 (d, 1H), 7.50 (d, 2H), 7.39 (d, 2H), 6.16 (s, 1H), 4.00 (m, 5H), 2.63 (s, 3H).

### EXAMPLE 15

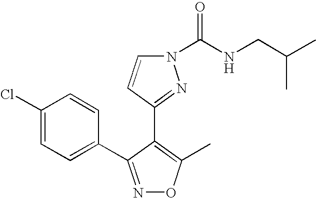

### 3-|3-(4-Chloro-phenyl)-5-methyl-isoxazol-4-yl|-pyrazole-1-carboxylic acid isobutyl-amide

The title compound was prepared analogously to 3-[3-(4-Chloro-phenyl)-5-methyl-isoxazol-4-yl]-pyrazole-1-carboxylic acid sec-butylamide (Example 4), where isobutylamine was substituted for sec-butylamine in the final step of that sequence. ¹H NMR (400 MHz, CDCl₃) δ 8.21 (s, 1H), 7.51 (d, 1H), 7.38 (d, 2H), 7.03 (m, 1H), 6.18 (s, 1H), 3.26 (t, 2H), 2.62 (s, 3H), 1.90 (m, 1H), 0.98 (d, 6H).

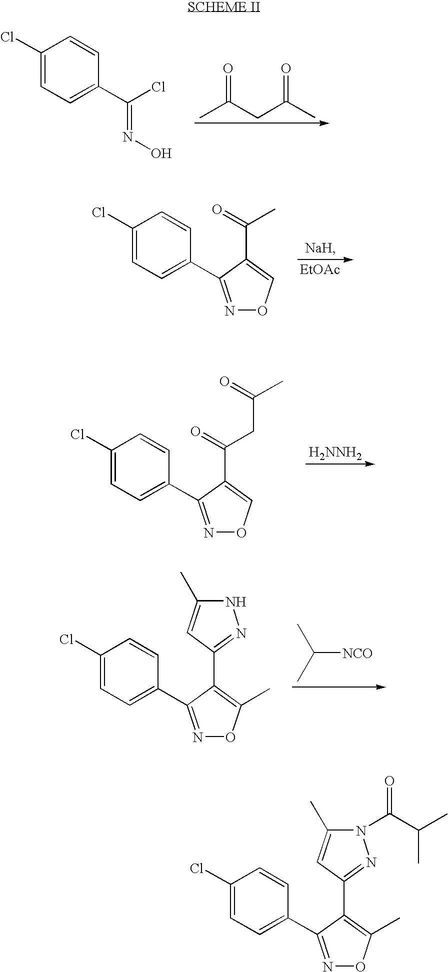

### EXAMPLE 16

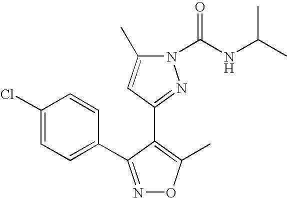

### 2-|3-(4-Fluoro-phenyl)-5-methyl-isoxazol-4-yl|-thiazole-4-carboxylic acid isopropylamide

Step 1

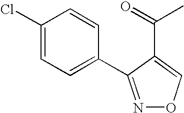

### 1-|3-(4-Chloro-phenyl)-isoxazol-4-yl|-ethanone

The title compound was prepared analogously to 1-(5-Methyl-3-phenyl-isoxazol-4-yl)-ethanone (described in step 1 of Example 1), where a-chloro-4-chlorobenzoyl oxime was substituted for a-chlorobenzoyl oxime in step 1 of that sequence.
Step 2

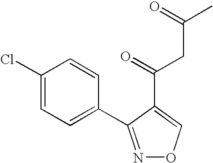

### 1-|3-(4-Chloro-phenyl)-isoxazol-4-yl|-butane-1,3-dione

A 100 mL round bottom flask was charged with 1-[3-(4-Chloro-phenyl)-isoxazol-4-yl]-ethanone (2.21 g, 10 mmol), NaH (253 mg, 11 mmol) and THF. The resulting mixture was stirred for 30 min at room temperature, under nitrogen. This mixture was treated with EtOAc (1.95 mL, 20 mmol), and stirred for 3 hr at room temperature. Reaction progress was monitored by TLC (10% EtOAc/Petroleum ether). Work-up: the mixture was diluted with EtOAc, washed with 1N HCL, NaHCO₃ (aq), brine, dried with MgSO₄, concentrated and chromatographed (10% EtOAc/Petroleum ether) to give yellow solid (2.28 g, 87%).
Step 3 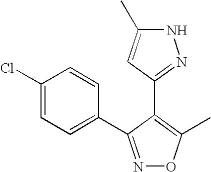
3-(4-Chloro-phenyl)-5-methyl-4-(5-methyl-1H-pyrazol-3-yl)-isoxazole: A round bottom flask was charged with 1-[3-(4-Chloro-phenyl)-isoxazol-4-yl]-butane-1,3-dione (0.8 g, 2.88 mmol) and EtOH (10 mL). To this solution was added a solution of hydrazine hydrate and EtOH (5 mL). The resulting solution stirred for 4 hours at room temperature. Work-up: the reaction was concentrated, dissolved in chloroform, washed with 1N HCl, NaHCO₃ (aq), brine, dried with MgSO₄, concentrated, chromatographed (25% AcOEt/Petroleum ether) to give the product (0.7 g, 2.56 mmol, 88% yield).
Step 4 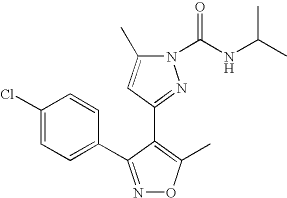
3-|3-(4-Chloro-phenyl)-5-methyl-isoxazol-4-yl|-5-methyl-pyrazole-1-carboxylic acid isopropylamide: The title compound was prepared analogously to Example 1 where 3-(4-Chloro-phenyl)-5-methyl-4-(5-methyl-1H-pyrazol-3-yl)-isoxazole was substituted for 5-Methyl-3-phenyl-4-(1H-pyrazol-3-yl)-isoxazole in step 4 of that sequence. ¹H NMR (400 MHz, CDCl₃) δ 7.50 (s, 1H), 7.38 (d, 2H), 7.28 (d, 2H), 6.82 (m, 1H), 3.77 (m, 1H), 2.58 (s, 3H), 1.25 (d, 6H), 1.15 (s, 3H).

### EXAMPLE 17

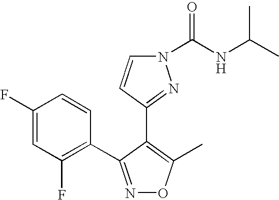

### 3-|3-(2,4-Difluoro-phenyl)-5-methyl-isoxazol-4-yl|-pyrazole-1-carboxylic acid isopropylamide

The title compound was prepared analogously to Example 1, where 2,4-difluoro-benzaldehyde was substituted for benzaldehyde in step 1 of that sequence. 2,4-difluoro-benzaldehyde was prepared as shown below. ¹H NMR (400 MHz, CDCl₃) δ 8.15 (s, 1H), 7.50 (m, 1H), 7.00 (m, 1H), 6.89 (m, 1H), 6.62 (m, 1H), 6.14 (s, 1H), 4.07 (septet, 1H), 2.69 (s, 3H), 1.25 (d, 6H).

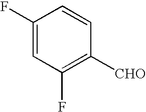

### 2,4-difluoro-benzaldehyde

A 1 L round bottom flask was charged with bromo-2,4-difluorobenzene (60.8 g, 0.315 mol), and anhydrous ethyl ether (400 mL), under a nitrogen atmosphere. The mixture was cooled to −78° C., where BuLi (2.87M, 0.315 mol) in hexane (110 mL) was added drop wise, maintain the internal temperature below −65° C. After addition of BuLi DMF (145 g, 1.987 mol) was added drop wise, followed by stirring for 30 min at this temperature. The temperature was allowed to warm to room temperature and stirred overnight. Work-up: the reaction was adjusted to pH=7 with 5% HCl, separated, dried over Na₂SO₄, concentrated, and distilled under reduced pressure (2 mm Hg). The fraction boiling between 82-88° C. was collected, giving the product as an oil (30.7 g, 68.7%).

### EXAMPLE 18

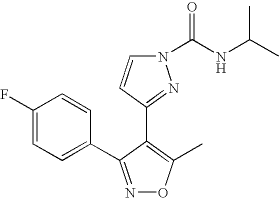

### 3-|3-(4-Fluoro-phenyl)-5-methyl-isoxazol-4-yl|-pyrazole-1-carboxylic acid isopropylamide

The title compound was prepared analogously to Example 1, where 4-fluoro-benzaldehyde was substituted for benzaldehyde in step 1 of that sequence. ¹H NMR (400 MHz, CDCl₃) δ 8.22 (s, 1H), 7.56 (m, 2H), 7.13 (t, 2H), 6.81 (m, 1H), 4.16 (septet, 1H), 2.65 (s, 3H), 1.32 (d, 6H).

### EXAMPLE 19

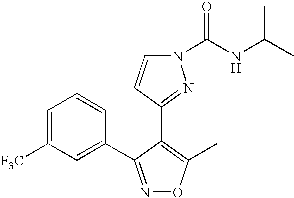

### 3-|5-Methyl-3-(3-trifluoromethyl-phenyl)-isoxazol-4-yl|-pyrazole-1-carboxylic acid isopropylamide

The title compound was prepared analogously to Example 1, where 3-trifluormethyl-benzaldehyde was substituted for benzaldehyde in step 1 of that sequence. ¹H NMR (400 MHz, CDCl₃) δ 8.23 (s, 1H), 7.96 (s, 1H), 7.76 (m, 1H), 7.71 (m, 1H), 7.54 (m, 1H), 6.72 (m, 1H), 6.20 (s, 1H), 4.13 (septet, 1H), 2.63 (s, 3H), 1.27 (d, 6H).

### EXAMPLE 20

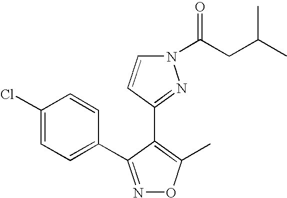

### 1-{3-|3-(4-Chloro-phenyl)-5-methyl-isoxazol-4-yl|-pyrazol-1-yl}-3-methyl-butan-1-one

The title compound was prepared analogously to Example 2, where 3-methyl-butyryl chloride was substituted for isopropyl isocyanate in final step of that sequence. ¹H NMR (400 MHz, CDCl₃) δ 8.24 (s, 1H), 7.53 (d, 2H), 7.41 (d, 2H), 6.19 (s, 1H), 2.98 (d, 2H), 2.68 (s, 3H), 2.26 (d, 1H), 1.20 (m, 1H), 1.05 (d, 6H).

### EXAMPLE 21

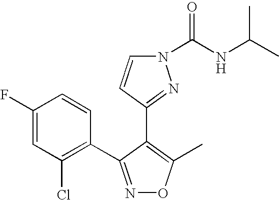

### 3-|3-(2-Chloro-4-fluoro-phenyl)-5-methyl-isoxazol-4-yl|-pyrazole-1-carboxylic acid isopropylamide

The title compound was prepared analogously to Example 1, where 2-chloro-4-fluoro-benzaldehyde was substituted for benzaldehyde in first step of that sequence. ¹H NMR (400 MHz, CDCl₃) δ 8.13 (s, 1H), 7.46 (m, 1H), 7.24 (m, 1H), 7.13 (m, 1H), 6.59 (m, 1H), 6.06 (s, 1H), 4.08 (septet, 1H), 2.73 (s, 3H), 1.26 (d, 6H).

### EXAMPLE 22

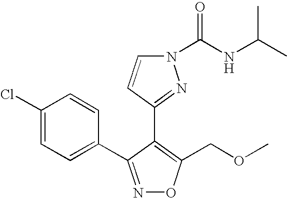

### 3-|3-(4-Chloro-phenyl)-5-methoxymethyl-isoxazol-4-yl|-pyrazole-1-carboxylic acid isopropylamide

The title compound was prepared analogously to Example 1, where 1-[3-(4-Chloro-phenyl)-5-methoxymethyl-isoxazol-4-yl]-ethanone was substituted for 2-[3-(4-Fluoro-phenyl)-5-methyl-isoxazol-4-yl]-thiazole-4-carboxylic acid isopropylamide in step 1 of that sequence. 1-[3-(4-Chloro-phenyl)-5-methoxymethyl-isoxazol-4-yl]-ethanone was prepared as described below. ¹H NMR (400 MHz, CDCl₃) δ 8.17 (s, 1H), 7.47 (d, 2H), 7.36 (d, 2H), 6.69 (d, 1H), 6.25 (d, 1H), 4.67 (s, 2H), 4.07 (septet, 1H), 3.45 (s, 3H), 1.24 (d, 6H).
Step 1

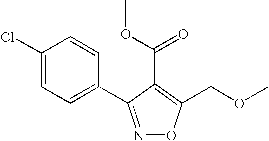

### 3-(4-Chloro-phenyl)-5-methoxymethyl-isoxazole-4-carboxylic acid methyl ester

A round bottom flask was charged with 4-methoxy-3-oxo-butyric acid methyl ester (0.4 g, 2.74 mmol, prepared as described in Tetrahedron (1986), 42(14), 3767-74), triethylamine (0.60 mL), and EtOH (20 mL). The mixture was cooled to 0° C., and treated with a-chloro-4-chlorobenzoyl oxime (0.51 g, 2.74 mmol) as a solution in EtOH (5 mL). The resulting solution was warmed to the room temperature and stirred overnight. Work-up: the solution was concentrated, dissolved in DCM washed with water, brine, dried Na₂SO₄, and concentrated. The crude material was purified by column chromatography on silica gel (EA: Petroleum ether=1:10) giving the product as a light yellow oil (0.2 g, 26%).
Step 2

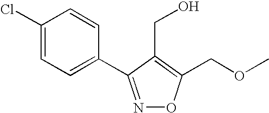

### |3-(4-Chloro-phenyl)-5-methoxymethyl-isoxazol-4-yl|-mEtOH

A 250 mL round bottom flask was charged with 3-(4-Chloro-phenyl)-5-methoxymethyl-isoxazole-4-carboxylic acid methyl ester (2.2 g, 7.20 mmol), and anhydrous ethyl ether (30 mL). The solution was cooled to 0° C., where LiAlH₄ (0.56 g, 1.44 mmol) was added carefully. The reaction was stirred at this temperature for two hours. Work-up: the reaction was quenched with water (2 mL), diluted with EtOAc, washed with 1N HCl, NaHCO₃ (aq.), brine, dried with MgSO₄, filtered, and concentrated to give the product as a yellow oil (1.2 g, 61%).
Step 3 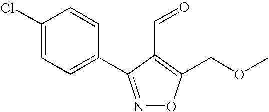
3-(4-Chloro-phenyl)-5-methoxymethyl-isoxazole-4-carbaldehyde: A 100 mL round bottom flask was charged with [3-(4-Chloro-phenyl)-5-methoxymethyl-isoxazol-4-yl]-mEtOH (0.166 g, 0.64 mmol), DCM (50 mL), and PCC (0.14 g, 9.69 mmol). The resulting mixture was stirred for 3 hours at the room temperature. Work-up: the mixture was concentrated and purified by column chromatography, eluting with DCM, to give the product as a white solid (0.12 g, 80%).
Step 4 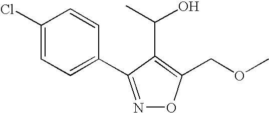
1-|3-(4-Chloro-phenyl)-5-methoxymethyl-isoxazol-4-yl|-EtOH: A round bottom flask was charged with Mg powder (0.15 g, 6.02 mmol), and anhydrous ethyl ether (3 mL). To this solution was added a solution CH₃I (1.3 g) and anhydrous ethyl ether (3 mL). After stirring until most Mg had dissolved, the resulting solution was added dropwise to a 100 mL three-necked round bottom flask containing 3-(4-Chloro-phenyl)-5-methoxymethyl-isoxazole-4-carbaldehyde (0.7 g, 3.01 mmol) and ethyl ether (6 mL). The resulting solution was stirred for 3 hours at room temperature. Work-up: the reaction was diluted ether, washed with 5% HCl (aq.), saturated brine, dried over Na₂SO₄, filtered, and concentrated to give yellow oil (0.7 g, 94.6%).
Step 5 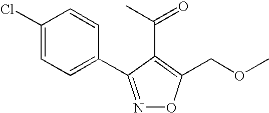
1-|3-(4-Chloro-phenyl)-5-methoxymethyl-isoxazol-4-yl|-ethanone: A round bottom flask was charged with 1-[3-(4-Chloro-phenyl)-5-methoxymethyl-isoxazol-4-yl]-EtOH (0.7 g), DCM (8 mL), and PCC (0.18 g, 5.38 mmol). The resulting mixture was stirred for 3 hours at the room temperature. The reaction was concentrated, and purified by column chromatography on silica gel (DCM eluent) giving the product as a yellow oil (0.44 g, 63.8%). ¹H NMR (400 MHz, CDCl₃) δ 7.52 (d, 2H), 7.37 (d, 2H), 6.79 (m, 1H), 6.25 (s, 1H), 4.85 (s, 1H), 4.04 (m, 1H), 3.47 (s, 3H), 2.61 (s, 3H), 1.24 (d, 6H).

### EXAMPLE 23

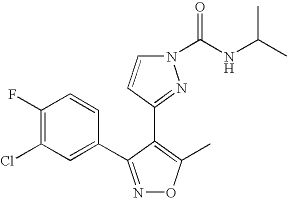

### 3-|3-(3-Chloro-4-fluoro-phenyl)-5-methyl-isoxazol-4-yl|-pyrazole-1-carboxylic acid isopropylamide

The title compound was prepared analogously to Example 1, where 4-fluoro-3-chloro-benzaldehyde was substituted for benzaldehyde in step 1 of that sequence. ¹H NMR (400 MHz, CDCl₃) δ 8.23 (s, 1H), 7.73 (m, 1H), 7.45 (m, 1H), 7.17 (t, 1H), 6.76 (m, 1H), 6.21 (s, 1H), 4.13 (septet, 1H), 2.61 (s, 3H), 1.28 (d, 6H).

### EXAMPLE 24

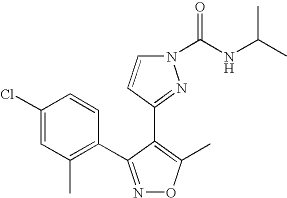

### 3-|3-(4-Chloro-2-methyl-phenyl)-5-methyl-isoxazol-4-yl|-pyrazole-1-carboxylic acid isopropylamide

The title compound was prepared analogously to Example 1, where 4-chloro-2-methyl-benzaldehyde was substituted for benzaldehyde in step 1 of that sequence. ¹H NMR (400 MHz, CDCl₃) δ8.09 (d, 1H), 7.25 (m, 3H), 6.56 (m, 1H), 5.96 (d, 1H), 4.05 (m, 1H), 2.73 (s, 3H), 3.15 (s, 3H), 1.25 (d, 6H).

### EXAMPLE 25

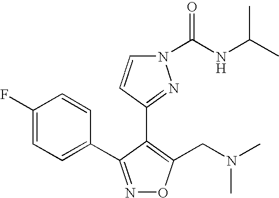

### 3-|5-Dimethylaminomethyl-3-(4-fluoro-phenyl)-isoxazol-4-yl|-pyrazole-1-carboxylic acid isopropylamide

The title compound was prepared analogously to Example 1, where 1-[5-Dimethylaminomethyl-3-(4-fluoro-phenyl)-isoxazol-4-yl]-ethanone was substituted for 1-(5-Methyl-3-phenyl-isoxazol-4-yl)-ethanone in step 1 of that sequence. 1-[5-Dimethylaminomethyl-3-(4-fluoro-phenyl)-isoxazol-4-yl]-ethanone was prepared as described below. ¹H NMR (400 MHz, CDCl₃) δ 8.23 (s, 1H), 7.57 (m, 2H), 7.15 (m, 2H), 6.95 (bs, 1H), 6.27 (s, 1H), 4.25 (m, 1H), 3.83 (bs, 2H), 2.47 (bs, 6H), 1.32 (d, 3H).
Step 1

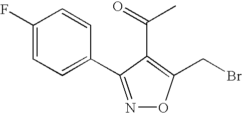

### 3-(4-Chloro-phenyl)-5-methoxymethyl-isoxazole-4-carboxylic acid methyl ester

A 1000 mL round bottom flask was charged with 1-[3-(4-Fluoro-phenyl)-5-methyl-isoxazol-4-yl]-ethanone (13.0 g, 0.060 mol), carbon tetrachloride (400 mL), AIBN (1.2 g, 7.3 mmol), and NBS (1.0 g, 0.062 mol). The resulting solution stirred overnight at 45° C. with illumination from a mercury vapor lamp. Reaction progress was monitored by TLC (EtOAc:petroleum ether=1:2). Work-up: the mixture was filtered, concentrated to give light red oil (15.0 g, 85.3%), that was used without further purification.
Step 2

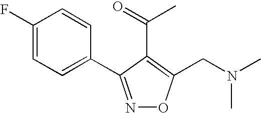

### |3-(4-Chloro-phenyl)-5-methoxymethyl-isoxazol-4-yl|-mEtOH

A 250 mL round bottom flask was charged with 3-(4-Chloro-phenyl)-5-methoxymethyl-isoxazole-4-carboxylic acid methyl ester (10.0 g, 0.033 mmol), EtOH (50 mL) and dimethylamine. The resulting solution was stirred for 30 minutes at the room temperature. Reaction progress was monitored by TLC (DCM:mEtOH=10:1). Work-up: the mixture was concentrated and purified by column chromatography (DCM:mEtOH=200:1), giving the product as a light brown oil (5.0 g, 56.8%).

### EXAMPLE 26

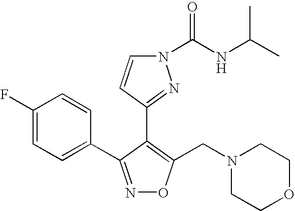

### 3-|3-(4-Fluoro-phenyl)-5-morpholin-4-ylmethyl-isoxazol-4-yl|-pyrazole-1-carboxylic acid isopropylamide

The title compound was prepared analogously to Example 25, where morpholine was substituted for dimethylamine in step 2 of that sequence. ¹H NMR (400 MHz, CDCl₃) δ 8.22 (s, 1H), 7.55 (m, 2H), 7.11 (m, 2H), 6.75 (bs, 1H), 6.27 (s, 1H), 4.13 (m, 1H), 3.88 (bs, 2H), 3.74 (bs, 4H), 2.59 (bs, 4H), 1.43 (d, 6H).

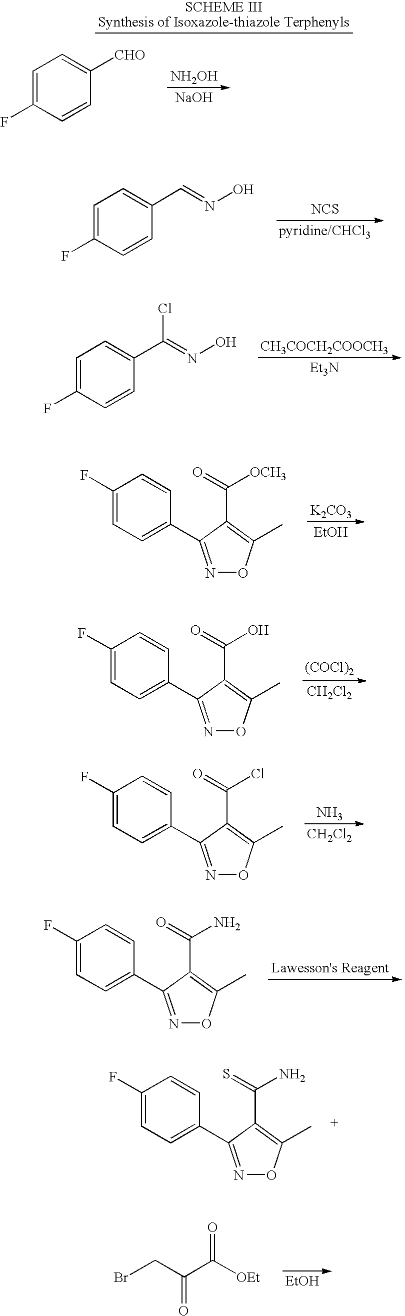

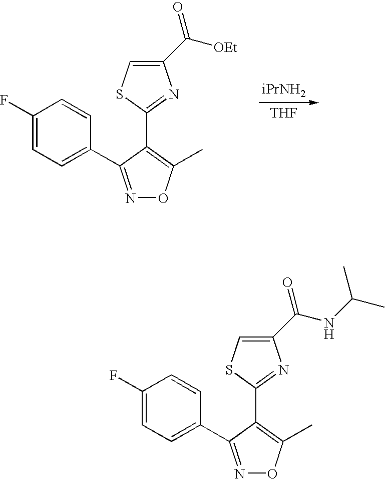

### EXAMPLE 27

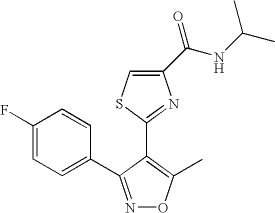

### 2-|3-(4-Fluoro-phenyl)-5-methyl-isoxazol-4-yl|-thiazole-4-carboxylic acid isopropylamide

Step 1

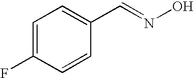

### 4-Fluoro-benzaldehyde oxime

A 500 mL 3-necked round bottom flask was charged with NH₂OH—HCl (22.41 g, 322.49 mmol), H₂O (50 mL), NaOH (12.9 g, 322.50 mmol) in H₂O (50 mL), and 4-fluorobenzaldehyde (20 g, 161.15 mmol), which was added drop-wise as a solution in EtOH (150 mL). The resulting solution stirred for 30 minutes at room temperature. The mixture was concentrated and dissolved in 30 mL of H₂O, which precipitates a white solid. The product was isolated by filtration, giving 21.5 g (96%) of 4-fluorobenzaldehyde oxime as a white solid.
Step 2

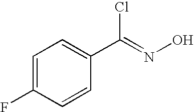

### 4-Fluoro-benzaldehyde chloro-oxime

A 1000 mL 3-necked round bottom flask was charged with 4-fluorobenzaldehyde oxime (134 g, 963.13 mmol), pyridine (9.6 g, 121.52 mmol), and CHCl₃ (500 mL). To the resulting solution was added NCS (141 g, 1.06 mol) in several batches. The solution was stirred for 8 hours at room temperature. The reaction progress was monitored by TLC (EtOAc/Petroleum ether=1:4). Work-up: the resulting mixture was washed 3 times with 120 mL of brine, dried over Na₂SO₄, and concentrated, giving 160 g (96%) of 4-fluorobenzoyl chloride oxime as a white solid.
Step 3

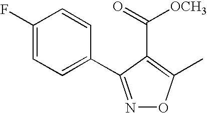

### 3-(4-Fluoro-phenyl)-5-methyl-isoxazole-4-carboxylic acid methyl ester

A 1000 mL round bottom flask was charged with methyl 3-oxobutanoate (93.7 g, 799.68 mmol), triethylamine (81.8 g, 801.80 mmol), and EtOH (500 mL). To the above was added 4-fluorobenzoyl chloride oxime (100 g, 518.73 mmol) in several batches, while maintaining a temperature of 5-10° C. The resulting solution was stirred for 3 hours at 50° C. Reaction progress was monitored by TLC (EtOAc/Petroleum ether=1:4). Work-up: the mixture was concentrated, dissolved in 500 mL of EtOAc, washed 3 times with 500 mL of saturated NaCl, dried over MgSO₄, and concentrated. The crude material was further purified by column chromatography with a 1:50 EtOAc/hexane, giving 25 g (19.5%) of product as white crystals.
Step 4

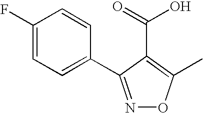

### 3-(4-Fluoro-phenyl)-5-methyl-isoxazole-4-carboxylic acid

A 250 mL round bottom flask was charged with methyl 3-(4-fluorophenyl)-5-methylisoxazole-4-carboxylate (10 g, 41.70 mmol), potassium hydroxide (7.1 g, 126.79 mmol), H₂O (50 mL), and EtOH (120 mL). The resulting solution was stirred overnight at reflux. Reaction progress was monitored by TLC (EtOAc/Petroleum ether=1:1, Rf=0.2). Work-up: the mixture was concentrated, dissolved in 30 mL of water, adjusted pH to 2 with HCl (10%). The resulting white solid was isolated by filtration, and dried in an oven under reduced pressure, resulting in 8.2 g (87%) of product as a white solid.
Step 5

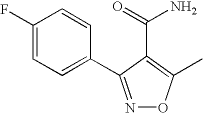

### 3-(4-Fluoro-phenyl)-5-methyl-isoxazole-4-carboxylic acid amide

A 250 mL round bottom flask was charged with 3-(4-Fluoro-phenyl)-5-methyl-isoxazole-4-carboxylic acid (5 g, 22.60 mmol) and CHCl₃ (100 mL). To this solution was added oxalyl chloride (8.61 g, 67.85 mmol), and DMF (3 drops). The resulting solution was stirred for 1 hour at room temperature and concentrated to an oil. The oil was dissolved in 100 mL of DCM, and treated with NH₃ (gas) for 2 hours at room temperature. Reaction progress was monitored by TLC (EtOAc/Petroleum ether=1:1). Work-up: product was isolated by filtration. The filter cake was washed 3 times with 20 mL of H₂O, and air dried, giving 4.54 g (90.8%) of product as a white solid.
Step 7

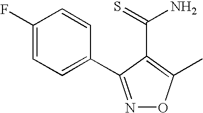

### 3-(4-Fluoro-phenyl)-5-methyl-isoxazole-4-carbothioic acid amide

A 250 mL 3-necked round bottom flask was charged with 3-(4-fluorophenyl)-5-methylisoxazole-4-carboxamide (4.5 g, 20.44 mmol), Lawesson's reagent (8.27 g, 20.45 mmol), and DME (100 mL). The resulting solution was stirred for 3 hours at 60° C. Work-up: the mixture was concentrated and purified by column chromatography with a 1:20 EtOAc/Petroleum ether, giving 5 g (97%) of product as a yellow solid.
Step 8 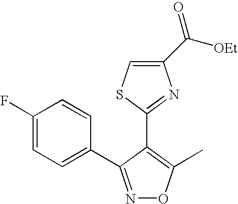
2-|3-(4-Fluoro-phenyl)-5-methyl-isoxazol-4-yl|-thiazole-4-carboxylic acid ethyl ester: A 250 mL round bottom flask was charged with 3-(4-Fluoro-phenyl)-5-methyl-isoxazole-4-carbothioic acid amide (5 g, 21.16 mmol) in EtOH (150 mL). To this was added ethyl 3-bromo-2-oxopropanoate (12.40 g, 63.59 mmol), and triethylamine (2.14 g, 21.19 mmol). The resulting solution stirred for 3 hours at 50° C. Work-up: the mixture was concentrated, dissolved in DCM (50 mL), washed 3 times with 30 mL of H₂O, dried over Na₂SO₄, concentrated, and purified by column chromatography with a 1:20 EtOAc/Petroleum ether. This resulted in 5.5 g (78.6%) of product as an orange solid.
Step 9

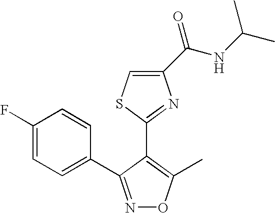

### 2-|3-(4-Fluoro-phenyl)-5-methyl-isoxazol-4-yl|-thiazole-4-carboxylic acid isopropylamide

A 200 mL sealed tube was charged with ethyl 2-[3-(4-Fluoro-phenyl)-5-methyl-isoxazol-4-yl]-thiazole-4-carboxylic acid ethyl ester (4.5 g, 13.54 mmol), and propan-2-amine (40 mL). The resulting solution was stirred overnight at 50° C. Work-up: the mixture was concentrated, purified by column chromatography with a 1:2 EtOAc/Petroleum ether. This gave 4.0 g (85.5%) of product as a white solid. ¹H NMR (400 MHz, CDCl₃) δ: 7.61 (t, 2H), 7.14 (t, 2H), 3.79 (s, 3H), 2.75 (s, 3H).

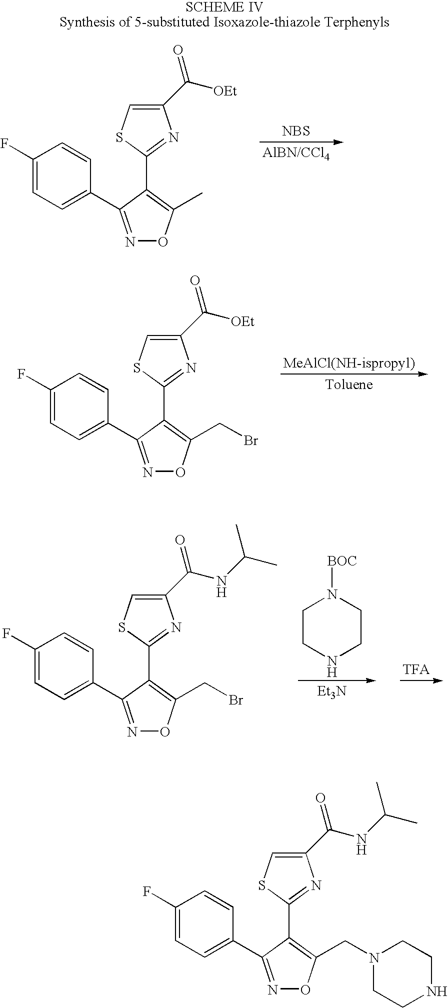

### EXAMPLE 28

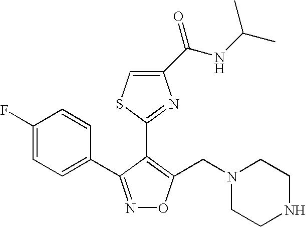

### 2-|3-(4-Fluoro-phenyl)-5-piperazin-1-ylmethyl-isoxazol-4-yl|-thiazole-4-carboxylic acid isopropylamide

Step 1

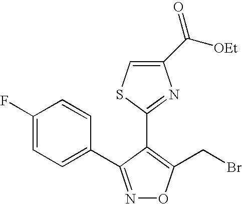

### 2-|5-Bromomethyl-3-(4-fluoro-phenyl)-isoxazol-4-yl|-thiazole-4-carboxylic acid ethyl ester

A 100 mL round bottom flask was charged with ethyl 2-[3-(4-Fluoro-phenyl)-5-methyl-isoxazol-4-yl]-thiazole-4-carboxylic acid ethyl ester (1.27 g, 3.82 mmol, prepared as described in Step 9, of Example 27, NBS (950 mg, 5.34 mmol), AIBN (100 mg, 0.61 mmol), in CCl₄ (50 mL). The resulting solution was stirred overnight at reflux. Reaction progress was monitored by reverse phase HPLC. Work-up: the resulting mixture was diluted with EtOAc (30 mL), washed 2 times with 30 mL of H₂O, dried over Na₂SO₄, filtered, and concentrated. This resulted in 1.67 g of crude product as a red-black solid, which was used in the next step without further purification.
Step 2

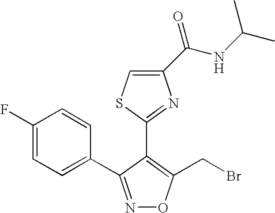

### 2-|5-Bromomethyl-3-(4-fluoro-phenyl)-isoxazol-4-yl|-thiazole-4-carboxylic acid isopropylamide

A 50 mL round bottom flask was charged with 2-[5-Bromomethyl-3-(4-fluoro-phenyl)-isoxazol-4-yl]-thiazole-4-carboxylic acid ethyl ester (822 mg, 2.0 mmol), MeAlCl(NH-ispropyl) (6.0 mL of 0.67 M solution, 4.0 mmol, prepared as described in Synthetic Communications, 12 (13), 989-993 (1982)), and toluene (6.0 mL). The resulting solution stirred for 1.5 hours at 80° C. Reaction progress was monitored by HPLC. Work-up: the reaction was diluted with DCM (20 mL), and stirred with Na₂SO₄10H₂O (10 g) for 1 hr, then filtered, and concentrated to a yellow oil (815 mg, 96%), which was used in the next step without further purification. LCMS (M+1^{+:} M+2⁺): 425.74, 427.26
Step 3

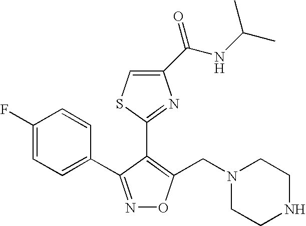

### 2-|3-(4-Fluoro-phenyl)-5-piperazin-1-ylmethyl-isoxazol-4-yl|-thiazole-4-carboxylic acid isopropylamide

A 50 mL round bottom flask was charged with 2-[5-Bromomethyl-3-(4-fluoro-phenyl)-isoxazol-4-yl]-thiazole-4-carboxylic acid isopropylamide (170 mg, 0.4 mmol), N-Boc-piperizine (90 mg, 0.48 mmol), Et₃N (278 μL, 0.8 mmol) and DMF (1.6 mL). The resulting solution stirred for 1 hour at room temperature. Reaction progress was monitored by TLC (1:1 EtOAc/Hex, Rf=0.4). The resulting solution was stripped of DMF under high vacuum, dissolved 1:1 TFA/DCM (5 mL), and stirred for 30 minutes at room temperature. Work-up: the reaction was diluted with toluene (5 mL), concentrated to an oil, and purified by C18 reverse phase HPLC, giving the product as a colorless foam (211 mg, 80%). ¹H NMR (400 MHz, CDCl₃) δ: 8.26 (m, 2H), 8.20 (s, 1H), 7.21 (t, 2H), 4.29 (septet, 1H), 3.83 (bs, 4H), 2.78 (bs, 1H), 3.03 (bs, 2H), 1.59 (s, 4H), 1.31 (d, 6H).

### EXAMPLE 29

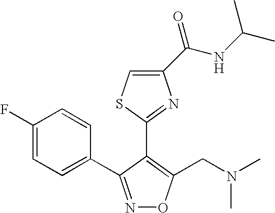

### 2-|5-Dimethylaminomethyl-3-(4-fluoro-phenyl)-isoxazol-4-yl|-thiazole-4-carboxylic acid isopropylamide

The title compound was prepared analogously to Example 28, where dimethylamine was substituted for N-Boc-piperizine in step 3 of that sequence. ¹H NMR (400 MHz, CDCl₃) δ: 8.00 (s, 1H), 7.04-7.49 (m, 4H), 6.94 (d, 1H), 4.10-4.17 (m, 1H), 3.85 (m, 2H), 2.36 (s, 6H), 1.13 (bs, 6H).

### EXAMPLE 30

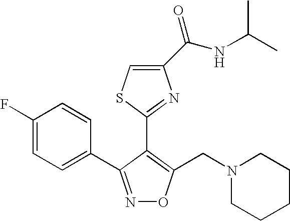

### 2-|3-(4-Fluoro-phenyl)-5-piperidin-1-ylmethyl-isoxazol-4-yl|-thiazole-4-carboxylic acid isopropylamide

A 50 mL round bottom flask was charged with 2-[5-Bromomethyl-3-(4-fluoro-phenyl)-isoxazol-4-yl]-thiazole-4-carboxylic acid isopropylamide (150 mg, 0.354 mmol), (preparation described in step 2 of Example 28), piperidine (42 μL, 0.424 mmol), Et₃N (100 μL, 0.71 mmol), and DMF (1.44 mL). The reaction stirred for 1 hour at room temperature, and progress was monitored by TLC (1:1 EtOAc/Hex). Workup: The reaction was neutralized with 1 M HCl (aq); diluted with toluene; concentrated to an oil; and purified via reverse phase HPLC, giving a colorless foam (78 mg, 52%). ¹H NMR (400 MHz, CDCl₃) δ: 8.06 (s, 1H), 8.01 (s, 1H), 7.82 (d, 1H)), 7.45 (m, 2H), 7.15 (m, 3H), 5.39 (s, 1H), 4.89 (s, 1H), 4.25 (m, 1H), 3.77 (bs, 1H), 3.38 (m, 2H), 2.81 (bs, 1H), 2.23 (bs, 1H)), 1.9 (bs, 1H), 1.59 (s, 3H), 1.21 (d, 6H). LCMS: 429.46 (M+1)⁺.

### EXAMPLE 31

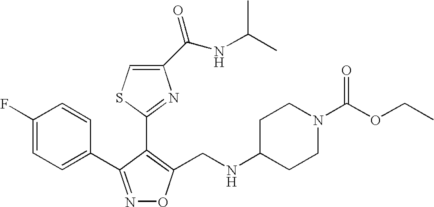

### 4-{|3-(4-isopropylcarbamoyl-thiazol-2-yl)-isoxazol-5-ylmethyl|-amino}-piperidine-1-carboxylic acid ethyl ester

The title compound was prepared analogously to Example 30, (2-[3-(4-Fluoro-phenyl)-5-piperidin-1-ylmethyl-isoxazol-4-yl]-thiazole-4-carboxylic acid isopropylamide)). ¹H NMR (400 MHz, CDCl₃) δ: 8.07 (s, 2H), 7.97 (s, 1H), 7.80 (d, 1H)), 7.41 (m, 3H), 7.15 (m, 3H), 5.37 (s, 1H), 4.89 (s, 1H), 4.61 (s, 2H), 4.23 (m, 3H), 4.09 (m, 2H), 3.42 (s, 1H), 3.38 (m, 2H), 3.15 (m, 1H), 2.71 (bs, 1H), 2.01 (d, 2H), 1.70 (m, 2H), 1.38 (s, 3H), 1.19 (d, 6H). LCMS: 515.74 (M+1)⁺.

### EXAMPLE 32

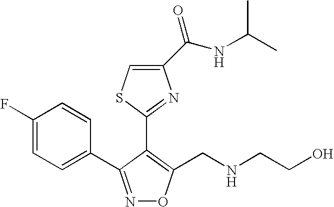

### 2-{3-(4-Fluoro-phenyl)-5-|(2-hydroxy-ethylamino)-methyl|-isoxazol-4-yl}-thiazole-4-carboxylic acid isopropylamide

The title compound was prepared analogously to Example 30, (2-[3-(4-Fluoro-phenyl)-5-piperidin-1-ylmethyl-isoxazol-4-yl]-thiazole-4-carboxylic acid isopropylamide). ¹H NMR (400 MHz, CDCl₃) δ: 8.00 (s, 1H), 7.52 (d, 1H), 7.43 (d, 2H), 7.12 (triplet, 2H), 4.64 (s, 2H), 4.23 (m, 1H), 3.92 (triplet, 2H), 3.27 (triplet, 2H), 1.18 (d, 6H). LCMS: 405.53 (M+1)⁺.

### EXAMPLE 33

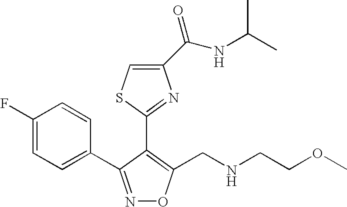

### 2-{3-(4-Fluoro-phenyl)-5-|(2-methoxy-ethylamino)-methyl|-isoxazol-4-yl}-thiazole-4-carboxylic acid isopropylamide

The title compound was prepared analogously to Example 30, (2-[3-(4-Fluoro-phenyl)-5-piperidin-1-ylmethyl-isoxazol-4-yl]-thiazole-4-carboxylic acid isopropylamide). ¹H NMR (400 MHz, CDCl₃) δ: 8.09 (s, 1H), 7.54 (m, 1H), 7.24 (m, 2H), 4.75 (s, 2H), 4.35 (m, 1H), 3.73 (triplet, 2H), 3.31 (s, 4H), 3.25 (triplet, 3H), 1.27 (d, 6H). LCMS: 420.54 (M+1)⁺.

### EXAMPLE 34

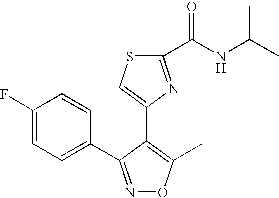

### 2-|3-(4-Fluoro-phenyl)-5-methyl-isoxazol-4-yl|-thiazole-4-carboxylic acid isopropylamide

Step 1

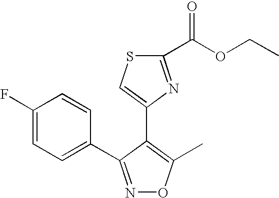

### 2-Bromo-1-|3-(4-fluoro-phenyl)-5-methyl-isoxazol-4-yl|-ethanone

A 50 mL round bottom flask was charged with 1-[3-(4-Fluoro-phenyl)-5-methyl-isoxazol-4-yl]-ethanone (1.5 g, 5.02 mmol, prepared as described in Journal of Medicinal Chemistry (1991), 34(2), 600-5), and EtOH (15 mL). The mixture was heated to reflux, where ethyl-2-amino-2-thioxoacetate (2.00 g, 15.0 mmol) was added. The resulting solution was stirred for 3 h at reflux. Reaction progress was monitored by TLC (EtOAc/Petroleum ether=1:4). Work-up: the mixture was concentrated to give 2.0 g yellow solid, that was purified by column chromatography (1:30 EtOAc/Petroleum ether), giving the product (1.38 g, 82.7%) as a white solid.
Step 2

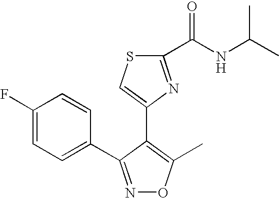

### 4-|3-(4-Fluoro-phenyl)-5-methyl-isoxazol-4-yl|-thiazole-2-carboxylic acid ethyl ester

A sealed tube was charged with 2-Bromo-1-[3-(4-fluoro-phenyl)-5-methyl-isoxazol-4-yl]-ethanone (0.4 g, 1.2 mmol), and isopropylamine (4 mL). The resulting solution was stirred for 3 hours at 40° C. Reaction progress was monitored by TLC (EtOAc/Petroleum ether=1:4). Work-up: the resulting solution was concentrated, and purified by column chromatography (1:10 EtOAc/Petroleum ether), giving the product (0.27 g, 65.8%) as a yellow solid. ¹H NMR (400 MHz, CDCl₃) δ: 7.63 (t, 2H), 7.13 (m, 2H), 4.24 (septet, 1H), 2.73 (s, 3H), 1.27 (d, 6H).

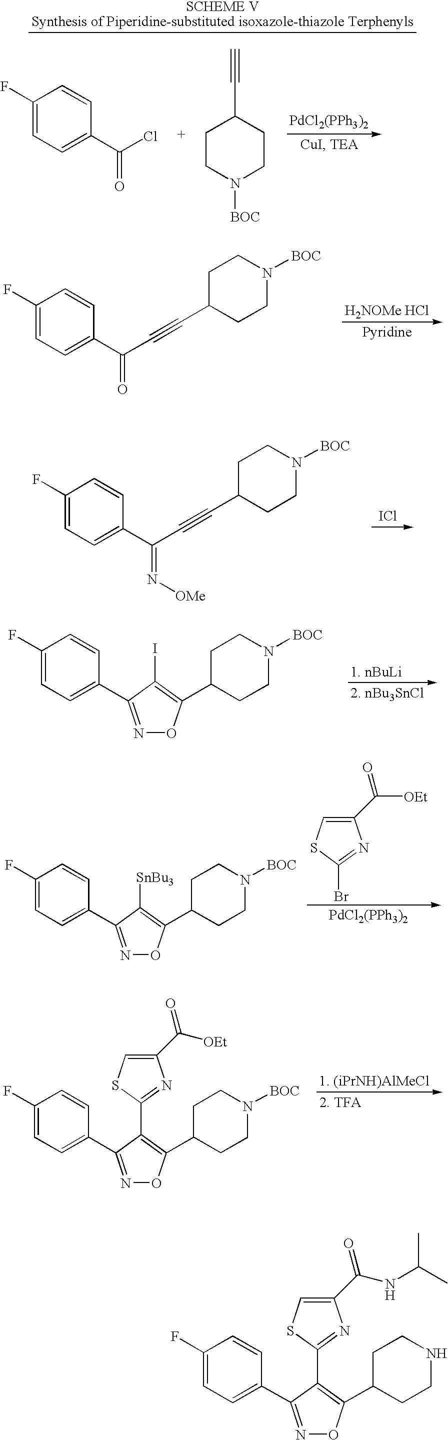

### EXAMPLE 35

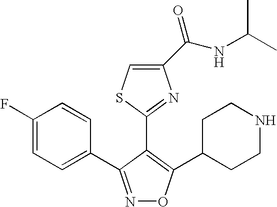

### 2-|3-(4-Fluoro-phenyl)-5-piperidin-4-yl-isoxazol-4-yl|-thiazole-4-carboxylic acid isopropylamide

Step 1

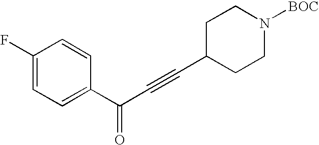

### 4-|3-(4-Fluoro-phenyl)-3-oxo-prop-1-ynyl|-piperidine-1-carboxylic acid tert-butyl ester

A 100 mL round bottom flask was charged with ethyl 4-ethynyl-piperidine-1-carboxylic acid tert-butyl ester (3.3 g, 15.8 mmol, prepared as described in J. Med. Chem. 2004, 47, 3111-3130 B. C. Raimundo et. al.), TEA (32 mL), PdCl₂(PPh₃)₂ (0.22 g, 0.32 mmol), and CuI (0.150 g, 0.79 mmol). This mixture was degassed and purged with N₂, then 4-Fluoro-benzoyl chloride (3.21 g, 20.5 mmol) was added dropwise at room temperature and allowed to stir at this temperature for 16 h. Conversion was monitored by TLC. The reaction was quenched with water, extracted with EtOAc (3×50 mL), washed with water (1×50 mL), brine (1×50 mL), dried over Na₂SO₄, filtered, and concentrated to give the product (5.2 g, 100%) as brown oil that was taken to next step without further purification. ¹H NMR (400 MHz, CDCl₃) δ: 8.20-8.13 (m, 2H), 7.23-7.14 (m, 2H), 3.82-3.76 (m, 2H), 3.27-3.15 (m, 2H), 2.92-2.86 (m, 1H), 1.79-1.73 (m, 2H), 1.62-1.55 (m, 2H), 1.47 (s, 9H).
Step 2

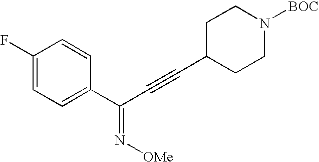

### 4-|3-(4-Fluoro-phenyl)-3-methoxyimino-prop-1-ynyl|-piperidine-1-carboxylic acid tert-butyl ester

A 100 mL round bottom flask was charged with ethyl 4-[3-(4-fluoro-phenyl)-3-oxo-prop-1-ynyl]-piperidine-1-carboxylic acid tert-butyl ester (5.2 g, 15.8 mmol), MeOH (31 mL), methoxyamine hydrochloride (1.8 g, 21.55 mmol), Na₂SO₄ (4.4 g, 31.6 mmol) and pyridine (3 mL), then stirred at room temperature for 7 h. Conversion was monitored by TLC. The reaction was quenched with water, extracted with EtOAc (3×100 mL), washed with water (1×50 mL), brine (1×50 mL), dried over Na₂SO₄ filtered, and concentrated in vacuo. The crude product was purified by silica gel (˜200 g) column chromatography with 0-20% EtOAc/Hexanes to afford the product (3.9 g, 69%). ¹H NMR (400 MHz, CDCl₃) δ: ¹H NMR (400 MHz, CDCl₃) δ: 8.24-8.10 (m, 2H), 7.16-7.12 (m, 2H), 4.06 (s, 3H), 3.78-3.70 (m, 2H), 3.31-3.26 (m, 2H), 2.96-2.91 (m, 1H), 1.77-1.69 (m, 2H), 1.62-1.57 (m, 2H), 1.47 (s, 9H); LCMS (M+1)⁺: 261.46
Step 3

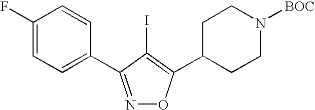

### 4-|3-(4-Fluoro-phenyl)-4-iodo-isoxazol-5-yl|-piperidine-1-carboxylic acid tert-butyl ester

A 100 mL round bottom flask was charged with 4-[3-(4-fluoro-phenyl)-3-methoxyimino-prop-1-ynyl]-piperidine-1-carboxylic acid tert-butyl ester (1 g, 2.77 mmol), and DCM (28 mL). To this solution was added dropwise iodine monochloride (0.54 mmol, 3.33 mL of 1 M solution in DCM). The resulting mixture was allowed to stir at room temperature for 3.5 h. Conversion was monitored by TLC. The reaction mixture was quenched with saturated aqueous solution of Na₂S₂O₃, extracted with EtOAc (3×100 mL), washed with water (1×50 mL), brine (1×50 mL), dried over Na₂SO₄, filtered, and concentrated. The resulting crude material was purified by silica gel (˜200 g) column chromatography with 0-20% EtOAc/Hexanes, giving the product as an off-white solid (0.8 g, 82%). ¹H NMR (400 MHz, CDCl₃) δ: 7.78-7.75 (m, 2H), 7.20-7.16 (m, 2H), 4.30-4.18 (m, 2H), 3.13-3.07 (m, 1H), 2.93-2.80 (m, 2H), 1.92-1.86 (m, 4H), 1.49 (s, 9H); LCMS (M+1-tBu)⁺: 417.23
Step 4

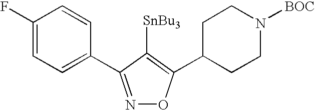

### 4-|3-(4-Fluoro-phenyl)-4-tributylstannanyl-isoxazol-5-yl|-piperidine-1-carboxylic acid tert-butyl ester

A 50 mL round bottom flask was charged with 4-[3-(4-fluoro-phenyl)-4-iodo-isoxazol-5-yl]-piperidine-1-carboxylic acid tert-butyl ester (1.8 g, 3.8 mmol), and 19 mL anhydrous THF, then cooled to −78° C., where n-BuLi (5.7 mmol, 3.5 mL of 1.6 M solution in hexanes) was added dropwise. The resulting mixture was stirred at this temperature for 30 min, then (Bu)₃SnCl (1.8 g, 5.7 mmol) was added dropwise via a syringe, and stirred for an 1 h at this temperature. Conversion was monitored by TLC. The reaction mixture was quenched with saturated aqueous Na₂S₂O₃, extracted with EtOAc (3×100 mL), washed with water (1×50 mL), brine (1×50 mL), dried over Na₂SO₄, filtered, and concentrated. The crude material was purified by silica gel (˜50 g) column chromatography with 0-10% EtOAc/Hexanes, giving the product as an off-white solid (0.95 g, 38%). ¹H NMR (400 MHz, CDCl₃) δ: 7.45-7.43 (m, 2H), 7.15-7.10 (m, 2H), 4.30-4.18 (m, 2H), 2.87-2.78 (m, 3H), 2.00-1.89 (m, 2H), 1.80-1.77 (m, 2H), 1.49 (s, 9H), 1.40-1.18 (m, 12H), 0.94-0.82 (m, 15H).
Step 5

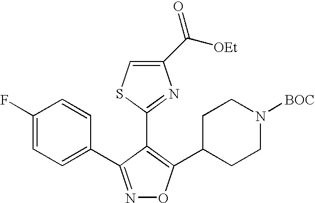

### 4-|4-(4-Ethoxycarbonyl-thiazol-2-yl)-3-(4-fluoro-phenyl)-isoxazol-5-yl|-piperidine-1-carboxylic acid tert-butyl ester

An 8 mL vial was charged with 2-bromothiazole-4-carboxylic acid ethyl ester (0.036 g, 0.15 mmol), 4-[3-(4-fluoro-phenyl)-4-tributyl-stannanyl-isoxazol-5-yl]-piperidine-1-carboxylic acid tert-butyl ester (0.1 g, 0.15 mmol), PdCl₂(PPh₃)₂ (0.011 g, 0.015 mmol), and anhydrous dioxane. The resulting mixture was heated to 110° C. and allowed to stir overnight. Conversion was monitored by TLC. The reaction mixture was concentrated in vacuo and purified by silica gel (˜50 g) column chromatography with 0-50% EtOAc/Hexanes, giving the product as off-white solid (0.070 g, 91%). ¹H NMR (400 MHz, CDCl₃) δ: 7.52-7.49 (m, 2H), 7.15-7.11, 4.42 (q, 2H), 4.28-4.18 (m, 2H), 3.55-3.53 (m, 1H), 2.90-2.80 (m, 2H), 2.10-1.84 (m, 4H), 1.48 (s, 9H), 1.41 (t, 3H); LCMS (M+1)⁺: 502.46.
Step 6

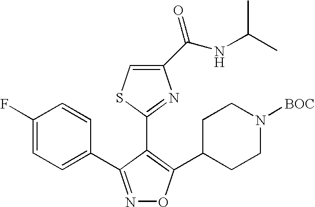

### 4-|3-(4-Fluoro-phenyl)-4-(4-isopropylcarbamoyl-thiazol-2-yl)-isoxazol-5-yl|-piperidine-1-carboxylic acid tert-butyl ester

An 8 mL vial was charged with 4-[4-(4-ethoxycarbonyl-thiazol-2-yl)-3-(4-fluoro-phenyl)-isoxazol-5-yl]-piperidine-1-carboxylic acid tert-butyl ester (0.07 g, 0.14 mmol), 0.5 mL anhydrous toluene, and MeAlCl(—NHiPr) (0.28 mmol, 0.42 mL of 0.67 M solution, prepared as described in Synthetic Communications, 1982, 12 (13), 989-993). The resulting solution was stirred for 1.5 hours at 80° C. The conversion was monitored by TLC. Work-up: the reaction was cooled, diluted with DCM (10 mL), and stirred with Na₂SO₄-10H₂O (1 g) for 1 hr, filtered, and concentrated to a yellow oil (0.06 g, 96%), which was used in the next step without further purification.
Step 7

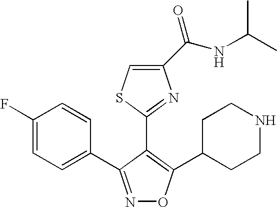

### 2-|3-(4-Fluoro-phenyl)-5-piperidin-4-yl-isoxazol-4-yl|-thiazole-4-carboxylic acid isopropylamide

A 20 mL vial was charged with 4-[3-(4-Fluoro-phenyl)-4-(4-isopropylcarbamoyl-thiazol-2-yl)-isoxazol-5-yl]-piperidine-1-carboxylic acid tert-butyl ester (0.06 g, 0.12 mmol), and dissolved in DCM (10 mL). To this solution was added 1 mL 1:1 mixture of TFA/DCM at room temperature. The resulting solution was stirred for 2 h at this temperature. Conversion was monitored by TLC. Work-up: the reaction concentrated and purified by C18 reverse phase column chromatography (10-60% MeCN/water with 0.1% TFA) to afford the product as white solid (32 mg, 67%). LCMS (M+1)⁺: 415.57

### EXAMPLE 36

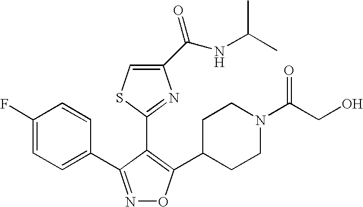

### 2-{3-(4-Fluoro-phenyl)-5-|1-(2-hydroxy-acetyl)-piperidin-4-yl|-isoxazol-4-yl}-thiazole-4-carboxylic acid isopropylamide

A 20 mL vial was charged with 2-[3-(4-fluoro-phenyl)-5-piperidin-4-yl-isoxazol-4-yl]-thiazole-4-carboxylic acid isopropylamide (50 mg, 0.095 mmol), DCM (1.9 mL), TEA (96 mg, 0.95 mmol), and acetoxyacetylchloride (20 mg, 0.14 mmol). The resulting solution was stirred at room temperature for 1 h. Conversion was monitored by TLC and LCMS. The reaction concentrated, dissolved in 1:1 THF/MeOH (1.8 mL), and stirred for 5 h at room temperature. Conversion was monitored by LCMS. Work-up: the reaction was quenched by Dowex acidic resin, stirred for 10 min and filtered. The filtrate was concentrated in vacuo and purified by C18 reverse phase column chromatography (20-60% MeCN/water with 0.1% TFA), giving the product as white solid (20 mg, 45%). ¹H NMR (400 MHz, CDCl₃) δ: ¹H NMR (400 MHz, CD₃OD) δ: 8.21 (s, 1H), 7.56-7.53 (m, 2H), 7.25-7.20, 4.61 (d, 1H), 4.34-4.14 (m, 3H), 3.90 (d, 1H), 3.76-3.68 (m, 1H), 3.20 (t, 1H), 2.88 (t, 1H), 2.10 (d, 2H), 1.99-1.83 (m, 2H), 1.25 (d, 6H); LCMS (M+1)⁺: 473.31.

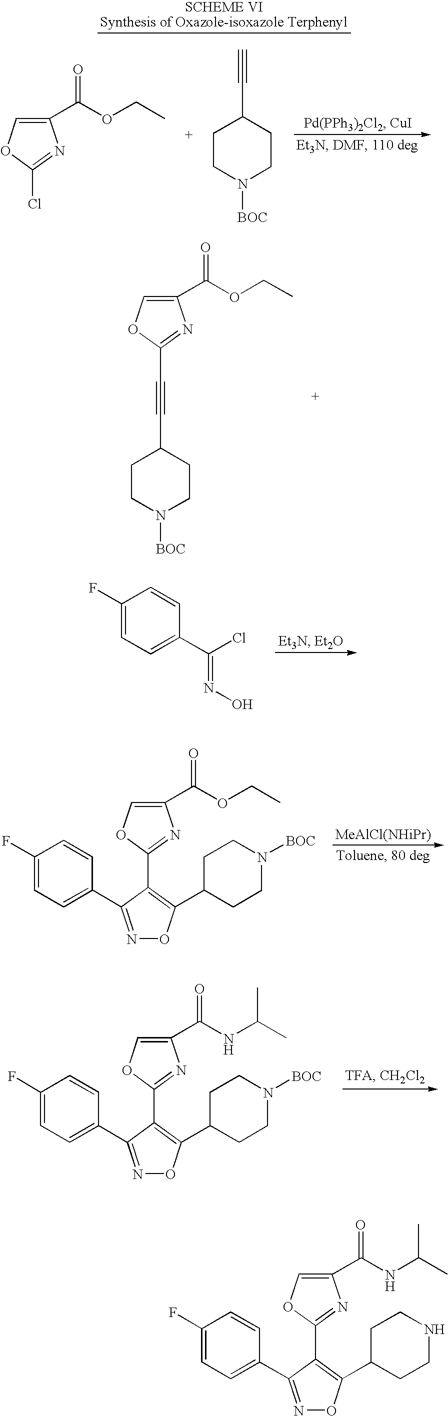

### EXAMPLE 37

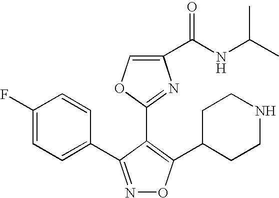

### 2-|3-(4-Fluoro-phenyl)-5-piperidin-4-yl-isoxazol-4-yl|-oxazole-4-carboxylic acid isopropylamide

Step 1 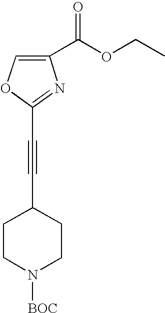
4-(4-Ethoxycarbonyl-oxazol-2-ylethynyl)-piperidine-1-carboxylic acid tert-butyl ester: A 50 mL round bottom flask was charged with 2-Chloro-oxazole-4-carboxylic acid ethyl ester (1.75 g, 10.0 mmol, prepared as described in Organic Letters (2002), 4(17), 2905-2907), 4-Ethynyl-piperidine-1-carboxylic acid tert-butyl ester (2.07 g, 10.0 mmol, prepared as described in Bioorganic & Medicinal Chemistry Letters (2004), 14(4), 947-952.), Pd(PPH₃)₂Cl₂ (350 mg, 0.50 mmol), CuI (190 mg, 1.00 mmol), Et₃N (5.0 mL), and DMF (15 mL). The resulting solution was vacuum-flushed with N₂, and then stirred for 2.0 hours at 110° C. Reaction progress was monitored by TLC (40% EtOAc/Hexane, Rf=0.4). Work-up: the mixture was concentrated, purified by column chromatography with 40% EtOAc/Hexane, resulting in 2.09 g (60%) of product as a brown oil.
Step 2

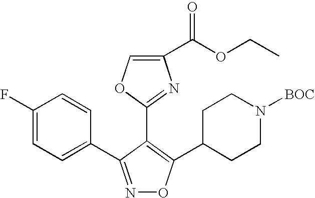

### 4-|4-(4-Ethoxycarbonyl-oxazol-2-yl)-3-(4-fluoro-phenyl)-isoxazol-5-yl|-piperidine-1-carboxylic acid tert-butyl ester

A 25 mL round bottom flask charged with methyl 4-(4-Ethoxycarbonyl-oxazol-2-ylethynyl)-piperidine-1-carboxylic acid tert-butyl ester (174.0 mg, 1.0 mmol), 4-Fluoro-benzaldehyde chloro-oxime (347.4 mg, 1.0 mmol), and 25% Et₃N/Et₂O (5 mL). The resulting solution was stirred at 50° C. for 2 days. Reaction progress was monitored by LCMS. Work-up: the mixture was concentrated, purified by C18 reverse phase HPLC, giving 29 mg (6%) of product as a white solid. LCMS (M+1⁺): 486.49
Step 3

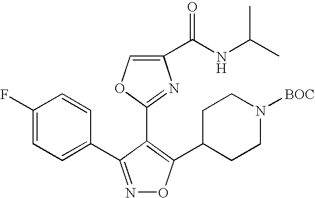

### 4-|3-(4-Fluoro-phenyl)-4-(4-isopropylcarbamoyl-oxazol-2-yl)-isoxazol-5-yl|-piperidine-1-carboxylic acid tert-butyl ester

A round bottom flask was charged with 4-[4-(4-Ethoxycarbonyl-oxazol-2-yl)-3-(4-fluoro-phenyl)-isoxazol-5-yl]-piperidine-1-carboxylic acid tert-butyl ester (70 mg, 0.144 mmol), MeAlCl(NH-ispropyl) (430 μl of 0.67 M solution, 0.288 mmol, prepared as described in Synthetic Communications, 12 (13), 989-993 (1982)), and toluene (430 μl). The resulting solution stirred for 2.5 hours at 75° C. Reaction progress was monitored by LCMS. Work-up: the reaction was diluted with DCM (10 mL), and stirred with Na₂SO₄-10H₂O (10 g) for 1 hr, then filtered, and concentrated to a light yellow solid, which was used in the next step without further purification. LCMS (M+1⁺): 499.53
Step 4

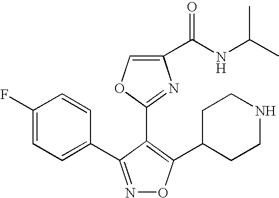

### 2-|3-(4-Fluoro-phenyl)-5-piperidin-4-yl-isoxazol-4-yl|-oxazole-4-carboxylic acid isopropylamide

A round bottom flask was charged with 4-[3-(4-Fluoro-phenyl)-4-(4-isopropylcarbamoyl-oxazol-2-yl)-isoxazol-5-yl]-piperidine-1-carboxylic acid tert-butyl ester (0.144 mmol crude from previous step) in 300% TFA/DCM (3 mL). The resulting solution stirred for 1 hour at room temperature. Reaction progress was monitored by LCMS. Work-up: the mixture was concentrated, purified by C18 reverse phase HPLC, giving 38 mg (52% for two steps, based on mass with 1 equivalent of TFA) of product as a white solid. ¹H NMR (400 MHz, CDCl₃) δ: 8.27 (s, 1H), 7.58 (dd, 2H), 7.18 (t, 2H), 6.65 (d, 1H), 6.10 (bs, 2H), 4.21 (septet, 1H), 3.75 (m, 1H), 3.65 (m, 2H), 3.23 (bs, 2H), 2.38 (bs, 4H), 1.25 (d, 6H). LCMS (M+1⁺): 399.86

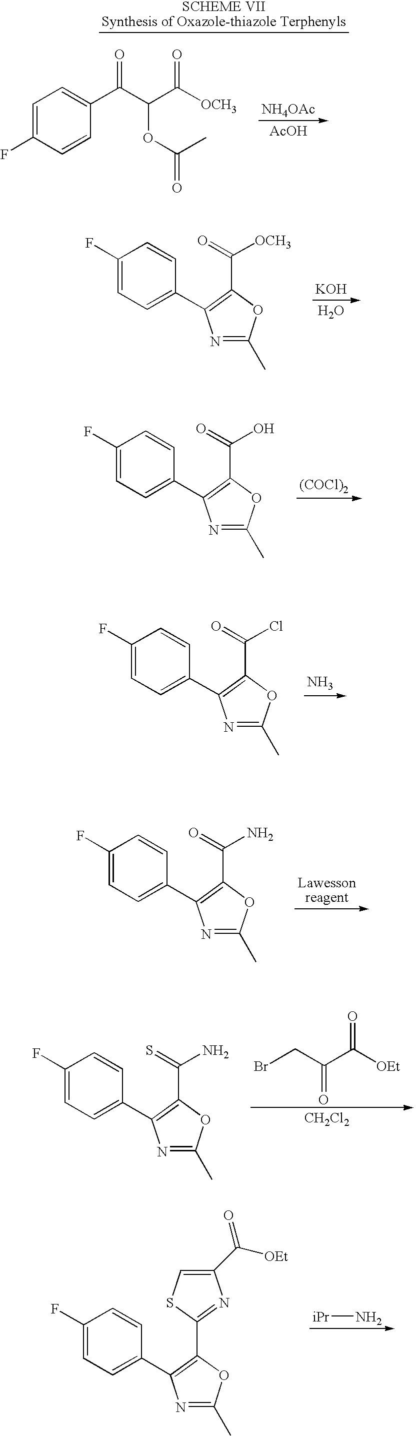

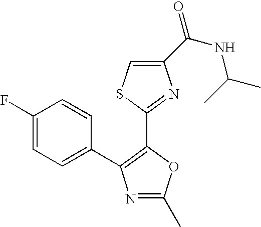

### EXAMPLE 38

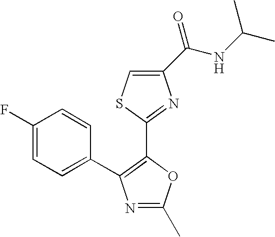

### 2-|5-(4-Fluoro-phenyl)-2-morpholin-4-ylmethyl-3H-imidazol-4-yl|-thiazole-4-carboxylic acid isopropylamide

Step 1 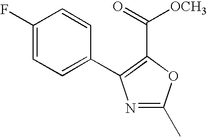
4-(4-Fluoro-phenyl)-2-methyl-oxazole-5-carboxylic acid methyl ester: A 250 mL round bottom flask was charged with 2-acetoxy-3-(4-fluoro-phenyl)-3-oxo-propionic acid methyl ester (25 g, 78.74 mmol, prepared as described in step 3 of Example 27), ammonium acetate (18.2 g, 234.00 mmol), and HOAc (30 mL). The resulting solution was stirred for 3.5 hours at 100° C. Reaction progress was monitored by TLC (EtOAc/Petroleum ether=1:4, Rf=0.4). Work-up: the mixture was concentrated, dissolved in 300 mL of EtOAc, washed 3 times with 200 mL of NaHCO₃ (10%), dried over Na₂SO₄, and concentrated. The resulting residue was purified by column chromatography with 1:20 EtOAc/Petroleum ether, resulting in 3.2 g (9%) of product as a yellow solid.
Step 2 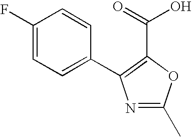
4-(4-Fluoro-phenyl)-2-methyl-oxazole-5-carboxylic acid: A 250 mL round bottom flask charged with methyl 4-(4-Fluoro-phenyl)-2-methyl-oxazole-5-carboxylic acid methyl ester (3.5 g, 14.60 mmol), potassium hydroxide (4.2 g, 75.0 mmol), H₂O (10 mL), and EtOH (30 mL). The resulting solution was refluxed for 40 minutes. Reaction progress was monitored by TLC (EtOAc/Petroleum ether=1:1). Work-up: the mixture was concentrated, and dissolved in 20 mL of H₂O, and adjusted to pH to 2 with HCl (10%). Product was isolated by filtration, resulting in 3.2 g (94%) of product as a white solid.
Step 3 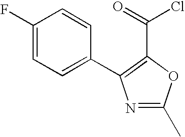
4-(4-Fluoro-phenyl)-2-methyl-oxazole-5-carbonyl chloride: A 250 mL round bottom flask was charged with 4-(4-Fluoro-phenyl)-2-methyl-oxazole-5-carboxylic acid (4.5 g, 19.95 mmol), oxalyl chloride (25.8 g, 203.26 mmol), and DCM (50 mL). To this was added N,N-dimethylformamide (catalytic amount). The resulting solution stirred for 4 hours at room temperature. Reaction progress was monitored by TLC (EtOAc/Petroleum ether=1:1). Work-up: the mixture was concentrated resulting in 4 g (67%) product as a yellow solid that was used without further purification.
Step 4 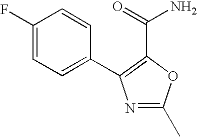
4-(4-Fluoro-phenyl)-2-methyl-oxazole-5-carboxylic acid amide: Into a 250 mL round bottom flask, was placed a solution of 4-(4-fluorophenyl)-2-methyloxazole-5-carbonyl chloride (4 g, 13.39 mmol) in DCM (50 mL). To the mixture was added ammonia gas (30 g, 1.76 mol). The resulting solution stirred for 3 hours at room temperature. Reaction progress was monitored by TLC (EtOAc/Petroleum ether=1:1). Work-up: solid product was filtered from the reaction and washed 2 times with 20 mL of H₂O, resulting in 3.4 g (92%) of product as a pale yellow solid.
Step 5 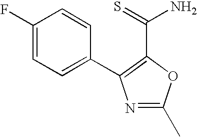
4-(4-Fluoro-phenyl)-2-methyl-oxazole-5-carbothioic acid amide: A 250 mL round bottom flask was charged with 4-(4-Fluoro-phenyl)-2-methyl-oxazole-5-carboxylic acid amide (3.4 g, 14.2 mmol), Lawesson's reagent (7.5 g, 18.56 mmol), and 1,2-dimethoxyethane (30 mL). The resulting solution was stirred for 10 at 60° C. Reaction progress was monitored by TLC (EtOAc/Petroleum ether=1:1). Work-up: the reaction was filtered. The filtrate was concentrated and purified by column chromatography with a 200:1 DCM/MeOH, giving 1.8 g (48%) of product as a yellow solid.
Step 6 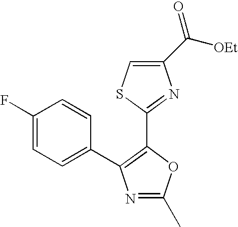
2-|4-(4-Fluoro-phenyl)-2-methyl-oxazol-5-yl|-thiazole-4-carboxylic acid ethyl ester: A 250 mL round bottom flask was charged with 4-(4-Fluoro-phenyl)-2-methyl-oxazole-5-carbothioic acid amide (1.8 g, 7.25 mmol), 3-bromo-2-oxopropanoate (7.4 g, 37.95 mmol), and EtOH (20 mL). The resulting solution stirred for 40 minutes at 60° C. Reaction progress was monitored by TLC (EtOAc/Petroleum ether=1:2). Work-up: the mixture was concentrated, dissolved in 100 mL of Et₂O, washed 2 times with 50 mL of H₂O, dried over Na₂SO₄, and concentrated to a yellow solid 1.0 g (39%).
Step 7 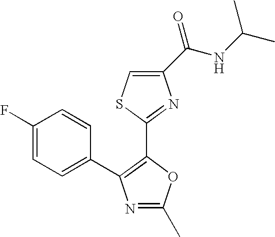
5-(4-Fluoro-phenyl)-2-methyl-3H-imidazole-4-carboxylic acid amide: A 100 mL sealed tube was charged with 2-[4-(4-Fluoro-phenyl)-2-methyl-oxazol-5-yl]-thiazole-4-carboxylic acid ethyl ester (1.5 g, 4.43 mmol), and propan-2-amine (5.3 g, 89.8 mmol). The resulting solution stirred for 6 hours at 50° C. Reaction progress was monitored by TLC (EtOAc/Petroleum ether=1:2). Work-up: the mixture was concentrated, dissolved in 100 mL of EtOAc, washed 2 times with 50 mL of H₂O, and dried over Na₂SO₄. The crude residue was purified by column chromatography with 1:10 EtOAc/Petroleum ether, resulting in 1.0 g (64%) of product as a white solid. ¹H NMR (400 MHz, CDCl₃) δ: 8.17 (m, 2H), 8.10 (s, 1H), 7.13 (t, 2H), 7.00 (m, 1H), 4.23 (septet, 1H), 2.62 (s, 3H), 1.27 (d, 6H).

### EXAMPLE 39

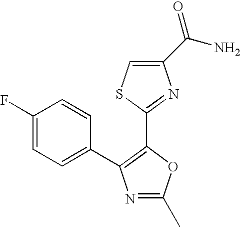

### 2-|4-(4-Fluoro-phenyl)-2-methyl-oxazol-5-yl|-thiazole-4-carboxylic acid amide

A 10 mL seal tube was charged with 2-[4-(4-Fluoro-phenyl)-2-methyl-oxazol-5-yl]-thiazole-4-carboxylic acid ethyl ester (25 mg, 0.075 mmol), ammonium hydroxide (2.5 mL), EtOH (1.5 mL), and DMSO (1.0 mL). The resulting solution stirred for 2 h at 120° C. Reaction progress was monitored by LCMS. Work-up: the mixture was concentrated, and purified by C18 semi-preparative HPLC, giving the product as a white solid 6 mg (26%). ¹H NMR (400 MHz, DMSO-d₆) δ: 8.41 (m, 2H), 8.23 (s, 1H), 7.28 (t, 2H), 6.91 (bs, 2H), 2.60 (s, 3H).

### EXAMPLE 40

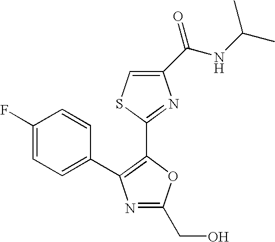

### 2-|4-(4-Fluoro-phenyl)-2-hydroxymethyl-oxazol-5-yl|-thiazole-4-carboxylic acid isopropylamide

Step 1 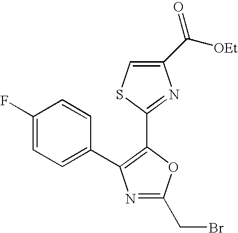
2-|2-Bromomethyl-4-(4-fluoro-phenyl)-oxazol-5-yl|-thiazole-4-carboxylic acid ethyl ester: A 100 mL round bottom flask was charged with 2-[4-(4-Fluoro-phenyl)-2-methyl-oxazol-5-yl]-thiazole-4-carboxylic acid ethyl ester (200 mg, 0.59 mmol, described in Step 6 of Example 38), NBS (120 mg, 0.67 mmol), AIBN (a catalytic amount), and CCl₄ (10 mL). The resulting solution stirred for 3 hours under light from a Hg vapor lamp at reflux. Reaction progress was monitored by TLC (EtOAc/Petroleum ether=1:4). Work-up: the resulting mixture was washed 2 times with 10 mL of H₂O, dried over Na₂SO₄, and purified by column chromatography with a 1:20 EtOAc/Petroleum ether. This gave 0.1 g (40%) of product as a pale yellow solid.
Step 2 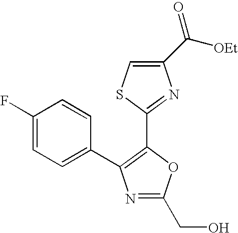
2-|4-(4-Fluoro-phenyl)-2-hydroxymethyl-oxazol-5-yl|-thiazole-4-carboxylic acid ethyl ester: A 50 mL round bottom flask was charged with ethyl 2-[2-Bromomethyl-4-(4-fluoro-phenyl)-oxazol-5-yl]-5 thiazole-4-carboxylic acid ethyl ester (500 mg, 0.61 mmol), DMSO (5 mL), and H₂O (2 mL). The resulting solution stirred overnight at 80° C. Reaction progress was monitored by TLC (EtOAc/Petroleum ether=1:1). Work-up: the reaction mixture diluted 10 mL of H₂O/ice, extracted two times with 50 mL of Et₂O, dried over Na₂SO₄, concentrated, and purified by column chromatography with 1:10 EtOAc/Petroleum ether. This gave 0.12 g (56%) of product as a pale yellow solid.
Step 3

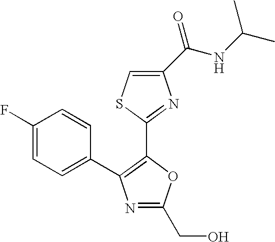

### 2-|4-(4-Fluoro-phenyl)-2-hydroxymethyl-oxazol-5-yl|-thiazole-4-carboxylic acid isopropylamide

A 10 mL sealed tube was charged with ethyl 2-[4-(4-Fluoro-phenyl)-2-hydroxymethyl-oxazol-5-yl]-thiazole-4-carboxylic acid ethyl ester (100 mg, 0.28 mmol), and propan-2-amine (170 mg, 2.88 mmol). The resulting solution stirred overnight at 50° C. Reaction progress was monitored by TLC (EtOAc/Petroleum ether=1:2). Work-up: the mixture was concentrated and purified by column chromatography with a 1:1 EtOAc/Petroleum ether. This gave 30 mg (29%) of the title compound as a pale yellow solid. ¹H NMR (400 MHz, CDCl₃) δ: 8.17 (m, 2H), 8.13 (s, 1H), 7.13 (t, 2H), 6.98 (m, 2H), 4.87 (s, 2H), 4.24 (septet, 1H), 1.26 (d, 6H).

### EXAMPLE 41

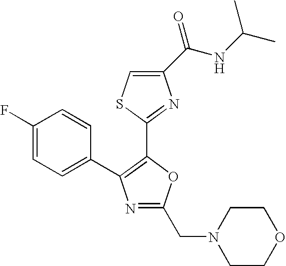

### 2-|4-(4-Fluoro-phenyl)-2-morpholin-4-ylmethyl-oxazol-5-yl|-thiazole-4-carboxylic acid isopropylamide

Step 1

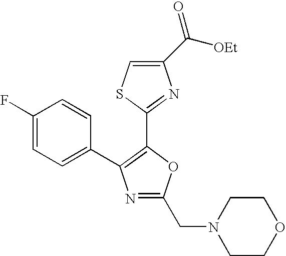

### 2-|4-(4-Fluoro-phenyl)-2-morpholin-4-ylmethyl-oxazol-5-yl|-thiazole-4-carboxylic acid ethyl ester

A 50 mL round bottom flask was charged with ethyl 2-(2-(bromomethyl)-4-(4-fluorophenyl)oxazol-5-yl)thiazole-4-carboxylate (200 mg, 0.49 mmol), morpholine (52 mg, 0.60 mmol), triethylamine (61 mg, 0.60 mmol), and EtOH (20 mL). The resulting solution was stirred for 1.5 hours at room temperature. Reaction progress was monitored by TLC (EtOAc/Petroleum ether=1:2). Work-up: the mixture was concentrated, dissolved in 30 mL of EtOAc, washed 3 times with 20 mL of brine, dried over Na₂SO₄ concentrated, resulting in 170 mg (84%) of product as yellow-red oil.
Step 2 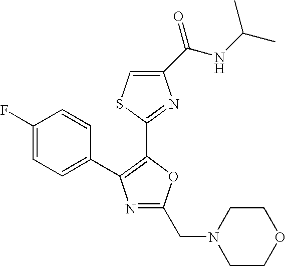
2-|4-(4-Fluoro-phenyl)-2-morpholin-4-ylmethyl-oxazol-5-yl|-thiazole-4-carboxylic acid isopropylamide: A 10 mL sealed tube was charged with 2-[4-(4-Fluoro-phenyl)-2-morpholin-4-ylmethyl-oxazol-5-yl]-thiazole-4-carboxylic acid ethyl ester (170 mg, 0.41 mmol), and propan-2-amine (3 mL). The resulting solution was stirred overnight at 60° C. Reaction progress was monitored by TLC (EtOAc/Petroleum ether=1:1). Work-up: the reaction mixture was concentrated, and purified by column chromatography with a 1:50 EtOAc/Petroleum ether, giving 30 mg (17%) of product as a yellow solid. ¹H NMR (400 MHz, CDCl₃) δ: 8.55 (bs, 2H), 8.34 (s, 1H), 7.63 (m, 2H), 7.18 (t, 2H), 4.15 (s, 2H), 4.09 (septet, 1H), 3.03 (bs, 4H), 2.71 (bs, 4H), 1.06 (d, 6H). LCMS (M+1⁺): 429.72

### EXAMPLE 42

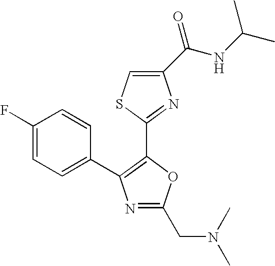

### 2-|2-Dimethylaminomethyl-4-(4-fluoro-phenyl)-oxazol-5-yl|-thiazole-4-carboxylic acid isopropylamide

The title compound was prepared analogously to Example 41, where dimethylamine was substituted for morpholine in step 2 of that sequence. ¹H NMR (400 MHz, CDCl₃) δ: 8.18 (m, 2H), 8.15 (s, 1H), 7.15 (t, 2H), 6.99 (m, 1H), 4.24 (m, 1H), 2.60 (bm, 2H), 1.54 (s, 6H), 1.27 (d, 6H).

### EXAMPLE 43

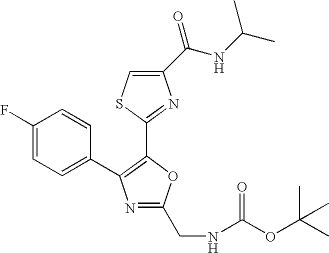

### |4-(4-Fluoro-phenyl)-5-(4-isopropylcarbamoyl-thiazol-2-yl)-oxazol-2-ylmethyl|-carbamic acid tert-butyl ester

The title compound was prepared analogously to Example 41, where sodium azide was substituted for morpholine in step 2 of that sequence. The resulting azide was reduced (RaNi/i-PrOH) and Boc protected. ¹H NMR (400 MHz, CDCl₃) δ: 8.17 (m, 2H), 8.13 (s, 1H), 7.14 (t, 2H), 7.00 (m, 1H), 5.22 (s, 1H), 4.56 (s, 2H), 4.24 (septet, 1H), 1.50 (s, 9H), 1.27 (d, 6H).

### EXAMPLE 44

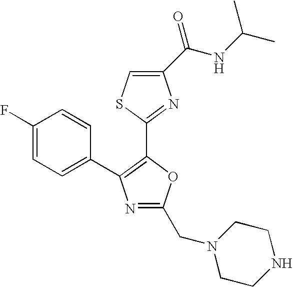

### 2-|4-(4-Fluoro-phenyl)-2-piperazin-1-ylmethyl-oxazol-5-yl|-thiazole-4-carboxylic acid isopropylamide

Step 1

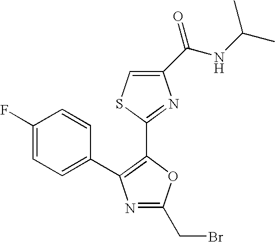

### 2-|2-Bromomethyl-4-(4-fluoro-phenyl)-oxazol-5-yl|-thiazole-4-carboxylic acid isopropylamide

A round bottom flask was charged with 2-[2-bromomethyl-4-(4-fluoro-phenyl)-oxazol-5-yl]-thiazole-4-carboxylic acid ethyl ester (300 mg, 0.73 mmol, prepared as described in Step 1 of Example 40), toluene (2 mL), and 2 mL of MeAlCl(NHiPr) (0.67 M solution in toluene, 1.46 mmol, prepared as described in Synth. Comm., 12 (13) 989-993.). The resulting mixture was warmed to 80° C. and left to stir for 2 hrs, then cooled to room temperature and poured in to a vigorously stirred slurry of sodium sulfate decahydrate (25 g) in DCM (100 mL). After 1 hr, the mixture was filtered, and the resulting filtrate was dried over MgSO₄, filtered, and concentrated in vacuo to afford the title compound (289 mg, 93% yield) as a tan solid that was determined to be sufficiently pure by available analytical methods to carry on to the next step. ¹H NMR (400 MHz, CDCl₃) δ 8.16 (m, 3H), 7.14 (m, 2H), 6.98 (m, 1H) 4.55 (s, 2H), 4.23 (m, 1H), 1.27 (d, 6H). LCMS: 423.7 (M+1)⁺.
Step 2

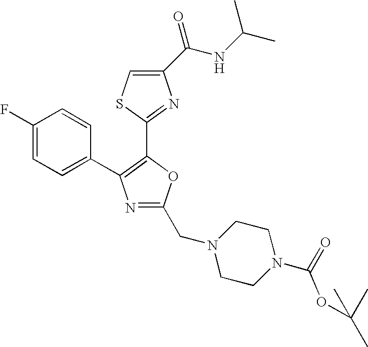

### 4-|4-(4-Fluoro-phenyl)-5-(4-isopropylcarbamoyl-thiazol-2-yl)-oxazol-2-ylmethyl|-piperazine-1-carboxylic acid tert-butyl ester

Cs₂CO₃ (861 mg, 2.65 mmol) was added to a stirred solution of 2-[2-bromomethyl-4-(4-fluoro-phenyl)-oxazol-5-yl]-thiazole-4-carboxylic acid isopropylamide (450 mg, 1.06 mmol), and tert-butyl 1-piperazinecarboxylate, (237 mg, 1.27 mmol) in DMF (22 mL) at room temperature. The resulting mixture was warmed to 80° C. and left to stir for 10 minutes, then cooled to room temperature and poured in to a separatory funnel containing 1:1 EtOAc:hexanes (200 mL) and 5% brine (100 mL). The organic layer was washed with an additional 3 portions of 5% brine (50 mL each), then dried over MgSO₄, filtered, and concentrated in vacuo. The resulting crude residue was purified by SiO₂ flash chromatography, eluting with 3:1 EtOAc:hexanes to afford the title compound (459 mg, 82%) as a white powder. ¹H NMR (400 MHz, CDCl₃) δ 8.16 (m, 2H), 8.12 (s, 1H), 7.12 (m, 2H), 6.98 (d, 1H), 4.22 (m, 1H), 3.83 (s, 2H), 3.49 (m, 4H), 2.61 (m, 4H), 1.44 (s, 9H), 1.26 (d, 6H). LCMS: 530.0 (M+1)⁺.
Step 3

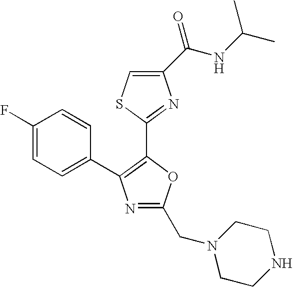

### 2-|4-(4-Fluoro-phenyl)-2-piperazin-1-ylmethyl-oxazol-5-yl|-thiazole-4-carboxylic acid isopropylamide

To a solution of 4-[4-(4-fluoro-phenyl)-5-(4-isopropylcarbamoyl-thiazol-2-yl)-oxazol-2-ylmethyl]-piperazine-1-carboxylic acid tert-butyl ester (350 mg, 0.66 mmol) in DCM (1 mL), was added 20% TFA in DCM (5 mL). After 1.25 hrs of stirring at room temperature, TLC analysis (70% EtOAc in hexanes) showed the disappearance of the Boc protected starting material. The resulting mixture was diluted with DCM (20 mL) and toluene (20 mL), and concentrated to dryness in vacuo. The crude residue was purified by automated C18 reverse phase semi-preparative HPLC to afford the title compound (220 mg, 61%, mono TFA salt) as an off white solid. ¹H NMR (400 MHz, CD₃OD) δ 8.28 (s, 1H), 8.19 (m, 2H), 7.70 (m, 1H), 7.23 (m, 2H), 4.17 (m, 1H), 3.99 (s, 2H), 3.28 (m, 4H), 2.94 (m, 4H), 1.26 (d, 6H). LCMS: 430.5 (M+1)⁺.

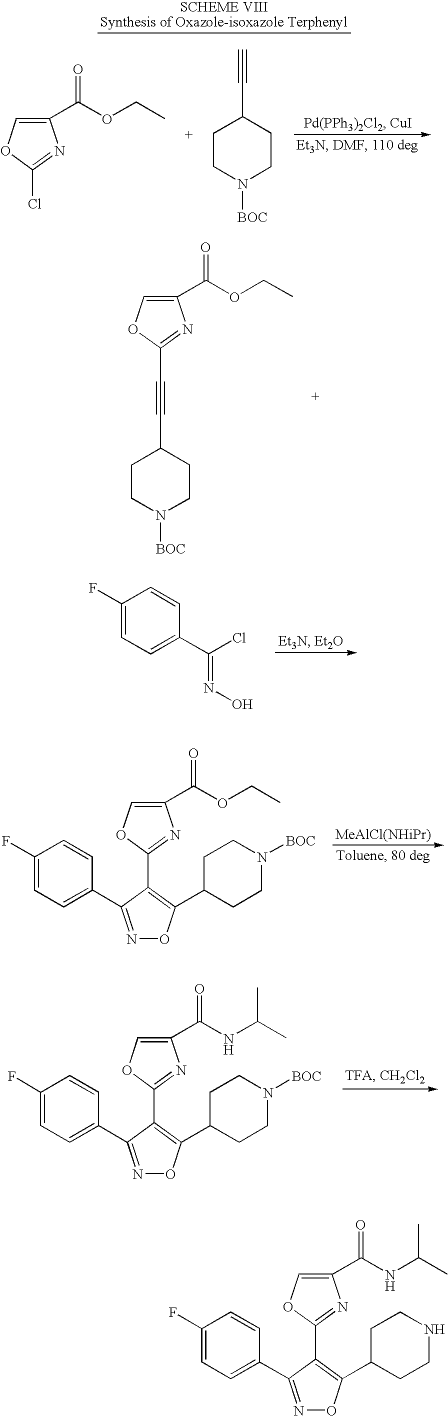

### EXAMPLE 45

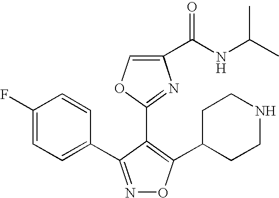

### 2-|3-(4-Fluoro-phenyl)-5-piperidin-4-yl-isoxazol-4-yl|-oxazole-4-carboxylic acid isopropylamide

Step 1

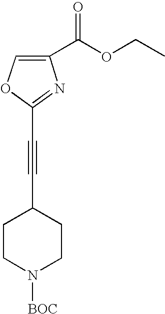

### 4-(4-Ethoxycarbonyl-oxazol-2-ylethynyl)-piperidine-1-carboxylic acid tert-butyl ester

A 50 mL round bottom flask was charged with 2-Chloro-oxazole-4-carboxylic acid ethyl ester (1.75 g, 10.0 mmol, prepared as described in Organic Letters (2002), 4(17), 2905-2907), 4-Ethynyl-piperidine-1-carboxylic acid tert-butyl ester (2.07 g, 10.0 mmol, prepared as described in Bioorganic & Medicinal Chemistry Letters (2004), 14(4), 947-952.), Pd(PPH₃)₂Cl₂ (350 mg, 0.50 mmol), CuI (190 mg, 1.00 mmol), Et₃N (5.0 mL), and DMF (15 mL). The resulting solution was vacuum-flushed with N₂, and then stirred for 2.0 hours at 110° C. Reaction progress was monitored by TLC (40% EtOAc/Hexane, Rf=0.4). Work-up: the mixture was concentrated, purified by column chromatography with 40% EtOAc/Hexane, resulting in 2.09 g (60%) of product as a brown oil.
Step 2

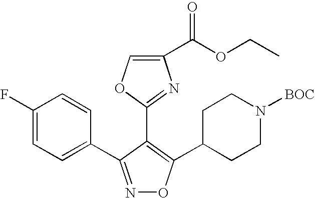

### 4-|4-(4-Ethoxycarbonyl-oxazol-2-yl)-3-(4-fluoro-phenyl)-isoxazol-5-yl|-piperidine-1-carboxylic acid tert-butyl ester

A 25 mL round bottom flask charged with methyl 4-(4-Ethoxycarbonyl-oxazol-2-ylethynyl)-piperidine-1-carboxylic acid tert-butyl ester (174.0 mg, 1.0 mmol), 4-Fluoro-benzaldehyde chloro-oxime (347.4 mg, 1.0 mmol), and 25% Et₃N/Et₂O (5 mL). The resulting solution was stirred at 50° C. for 2 days. Reaction progress was monitored by LCMS. Work-up: the mixture was concentrated, purified by C18 reverse phase HPLC, giving 29 mg (6%)of product as a white solid. LCMS (M+1⁺): 486.49
Step 3

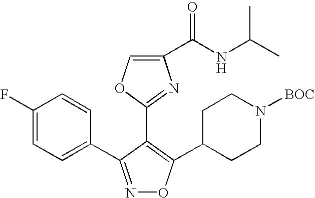

### 4-|3-(4-Fluoro-phenyl)-4-(4-isopropylcarbamoyl-oxazol-2-yl)-isoxazol-5-yl|-piperidine-1-carboxylic acid tert-butyl ester

A round bottom flask was charged with 4-[4-(4-Ethoxycarbonyl-oxazol-2-yl)-3-(4-fluoro-phenyl)-isoxazol-5-yl]-piperidine-1-carboxylic acid tert-butyl ester (70 mg, 0.144 mmol), MeAlCl(NH-ispropyl) (430 μl of 0.67 M solution, 0.288 mmol, prepared as described in Synthetic Communications, 12 (13), 989-993 (1982)), and toluene (430 μl). The resulting solution stirred for 2.5 hours at 75° C. Reaction progress was monitored by LCMS. Work-up: the reaction was diluted with DCM (10 mL), and stirred with Na₂SO₄-10H₂O (10 g) for 1 hr, then filtered, and concentrated to a light yellow solid, which was used in the next step without further purification. LCMS (M+1⁺): 499.53
Step 4

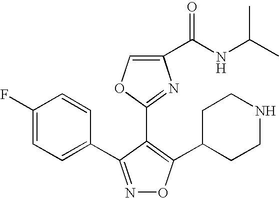

### 2-|3-(4-Fluoro-phenyl)-5-piperidin-4-yl-isoxazol-4-yl|-oxazole-4-carboxylic acid isopropylamide

A round bottom flask was charged with 4-[3-(4-Fluoro-phenyl)-4-(4-isopropylcarbamoyl-oxazol-2-yl)-isoxazol-5-yl]-piperidine-1-carboxylic acid tert-butyl ester (0.144 mmol crude from previous step) in 30% TFA/DCM (3 mL). The resulting solution stirred for 1 hour at room temperature. Reaction progress was monitored by LCMS. Work-up: the mixture was concentrated, purified by C18 reverse phase HPLC, giving 38 mg (52% for two steps, based on mass with 1 equivalent of TFA) of product as a white solid. ¹H NMR (400 MHz, CDCl₃) δ: 8.27 (s, 1H), 7.58 (dd, 2H), 7.18 (t, 2H), 6.65 (d, 1H), 6.10 (bs, 2H), 4.21 (septet, 1H), 3.75 (m, 1H), 3.65 (m, 2H), 3.23 (bs, 2H), 2.38 (bs, 4H), 1.25 (d, 6H). LCMS (M+1⁺): 399.86

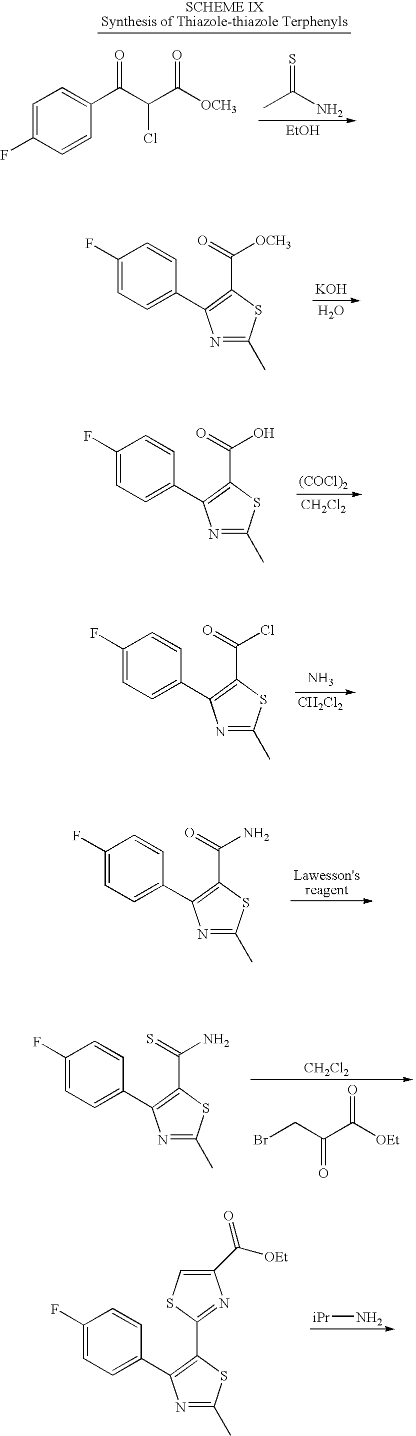

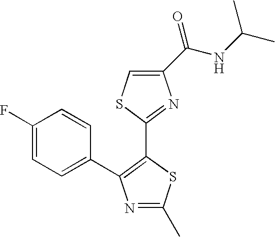

### EXAMPLE 46

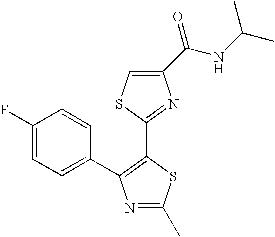

### 4′-(4-Fluoro-phenyl)-2′-methyl-|2,5′|bithiazolyl-4-carboxylic acid isopropylamide

Step 1

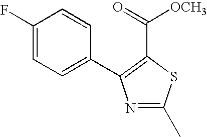

### 4-(4-Fluoro-phenyl)-2-methyl-thiazole-5-carboxylic acid methyl ester

A 500 mL round bottom flask was charged with 2-Chloro-3-(4-fluoro-phenyl)-3-oxo-propionic acid methyl ester (10 g, 0.043 mol, described in Step 2 of Example 27), and thioacetamide (5 g, 0.067 mol) and EtOH (250 mL). The resulting solution stirred overnight at reflux. Reaction progress %% as monitored by TLC (EtOAc/Petroleum ether=1:10). Work-up: the mixture was concentrated, dissolved in 100 mL of DCM, washed 2 times with 50 mL of water, dried over MgSO₄, and concentrated giving 8 g (73.4%) of product as a red solid.
Step 2

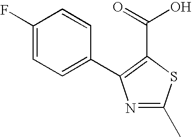

### 4-(4-Fluoro-phenyl)-2-methyl-thiazole-5-carboxylic acid

A 500 mL round bottom flask was charged with 4-(4-Fluoro-phenyl)-2-methyl-thiazole-5-carboxylic acid methyl ester (8 g, 0.032 mol), potassium hydroxide (5.35 g, 0.096 mol), H₂O (20 mL), and EtOH (100 mL). The resulting solution was stirred for 4 hours at reflux. Reaction progress was monitored by TLC (AcOEt/Petroleum ether=1:10). Work-up: the mixture was concentrated, dissolved in 100 mL of H₂O, washed 2 times with 50 mL of DCM, and adjusted to pH=2 with HCl (6N), which caused formation of a white precipitate. The precipitate was filtrated and dried to give 6.38 g (85%) of product as a white solid.
Step 3

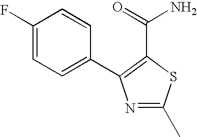

### 4-(4-Fluoro-phenyl)-2-methyl-thiazole-5-carboxylic acid amide

A 250 mL single-necked flask was charged with 4-(4-Fluoro-phenyl)-2-methyl-thiazole-5-carboxylic acid (3 g, 0.013 mol), oxalyl chloride (16.1 g, 0.13 mol), DMF (two drops), and DCM (100 mL). The resulting solution was stirred for 2 hours at room temperature. Reaction progress was monitored by TLC (EtOAc/Petroleum ether=1:1) after a MeOH quench. The crude acid chloride was concentrated, dissolved in 100 mL of DCM, and reacted with NH3(g) for 2 hours. Work-up: product was isolated by filtration. The filter cake was washed with water and dried to give 2.2 g (81%) of product as a red solid.
Step 4

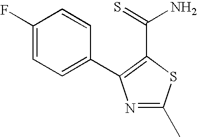

### 4-(4-Fluoro-phenyl)-2-methyl-thiazole-5-carbothioic acid amide

A 250 mL 3-necked flask was charged 4-(4-Fluoro-phenyl)-2-methyl-thiazole-5-carboxylic acid amide (2.2 g, 0.09 mol), DME (100 mL), and Lawesson's reagent (4.45 g, 0.011 mol). The resulting solution was stirred for 40 minutes at 60° C. Work-up: the mixture was concentrated to give yellow sticky solid, which was purified by column chromatography with a 1:50 EtOAc/Petroleum ether. This gave 1.7 g (72.3%) of product as a light yellow solid.
Step 5

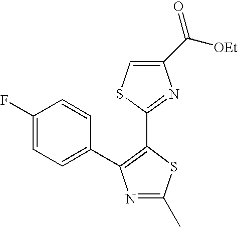

### 4′-(4-Fluoro-phenyl)-2′-methyl-|2,5′|bithiazolyl-4-carboxylic acid ethyl ester

A 100 mL round bottom flask was charged with 4-(4-Fluoro-phenyl)-2-methyl-thiazole-5-carbothioic acid amide (1.5 g, 0.006 mmol), 3-bromo-3-oxopropanoate (5.71 g, 0.03 mol), and DCM (50 mL). The resulting solution was stirred for 5 hours at reflux. Reaction progress was monitored by TLC (EtOAc/Petroleum ether=1:2). Work-up: the reaction mixture was concentrated, and purified by column chromatography with 20:1 EtOAc/Petroleum ether, giving 1.2 g (59%) of product as a yellow solid.
Step 6 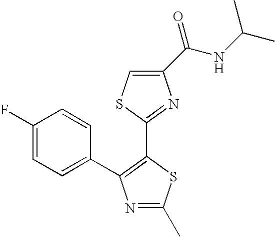
4′-(4-Fluoro-phenyl)-2′-methyl-|2,5′|bithiazolyl-4-carboxylic acid isopropylamide: A 10 mL sealed tube was charged with ethyl 4′-(4-Fluoro-phenyl)-2′-methyl-[2,5′]bithiazolyl-4-carboxylic acid ethyl ester (400 mg, 1.15 mmol), and isopropyl amine (7 mL). The resulting solution was stirred overnight at 50° C. Reaction progress was monitored by TLC (EtOAc/Petroleum ether=1:2). Work-up: the mixture was concentrated, and purified by column chromatography with a 1:5 EtOAc/Petroleum ether, giving 0.23 (55%) of the title compound as a white solid. ¹H NMR (400 MHz, CDCl₃) δ: 7.92 (s, 1H), 7.57 (m, 2H), 7.16 (t, 3H), 7.00 (d, 1H), 4.24 (septet, 1H), 2.80 (s, 3H), 1.27 (d, 6H).

### EXAMPLE 47

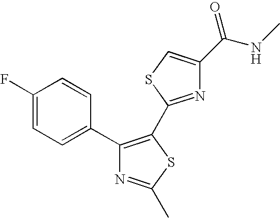

### 4′-(4-Fluoro-phenyl)-2′-methyl-|2,5′|bithiazolyl-4-carboxylic acid methylamide

Step 1

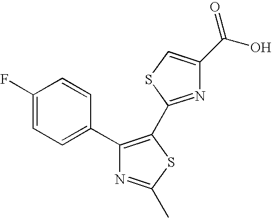

### 4′-(4-Fluoro-phenyl)-2′-methyl-|2,5′|bithiazolyl-4-carboxylic acid

To a stirred solution of 4′-(4-fluoro-phenyl)-2′-methyl-[2,5′]bithiazolyl-4-carboxylic acid ethyl ester (1.95 g, 5.6 mmol) in MeOH (20 mL) at room temperature was added LiOH (7.3 mL of a 1N aqueous solution, 7.3 mmol). The resulting mixture was warmed to 45° C. and left to stir for 2 hrs, at which time TLC analysis (1:1 EtOAc:hexanes) revealed the disappearance of the ester starting material. The reaction was cooled to room temperature and made acidic with the addition of HCl (10 mL of 1N aqueous solution) and further diluted with H₂O (200 mL). The resulting heterogeneous mixture %% as washed with EtOAc (6×100 mL portions) and the combined organic extracts were dried (MgSO₄), filtered, and concentrated in vacuo to obtain the title compound (1.68 g, 5.3 mmol, 95% yield) as a white powder. LCMS: 320.7 (M+1)⁺, 319.1 (M−1)⁻.
Step 2

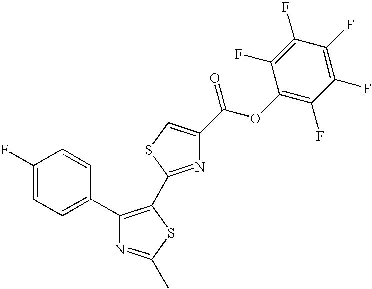

### 4′-(4-Fluoro-phenyl)-2′-methyl-|2,5′|bithiazolyl-4-carboxylic acid pentafluorophenyl ester

To a stirred solution of 4′-(4-fluoro-phenyl)-2′-methyl-[2,5′]bithiazolyl-4-carboxylic acid (1.68 g, 5.3 mmol), and pyridine (466 μL, 5.8 mmol) in DMF (50 mL), at room temperature, was added pentafluorophenyl trifluoroacetate (1.1 mL, 6.3 mmol). After 1 hr, the reaction was determined to be complete by TLC analysis (3:1 hexanes:EtOAc). The mixture was then poured in to a separatory funnel containing 1:1 hexanes:EtOAc (300 mL) and washed with aqueous HCl (50 mL, 0.1 N), 5% brine (4×50 mL), 5% NaHCO₃ (50 mL), and H₂O (100 mL). The organic layer was dried over MgSO₄, filtered, and concentrated to dryness in vacuo to afford the title compound (2.54 g, 99% yield) as a pale yellow solid. ¹H NMR (400 MHz, CDCl₃) δ 8.27 (s, 1H), 7.55 (m, 2H), 7.17 (m, 2H), 2.78 (s, 3H). LCMS: 486.7 (M+1)⁺.
Step 3

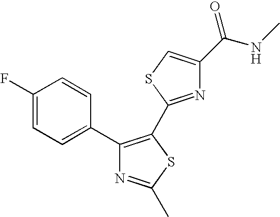

### 4′-(4-Fluoro-phenyl)-2′-methyl-|2,5′|bithiazolyl-4-carboxylic acid methylamide

Methylamine (150 μL of a 2.0 M solution in THF, 0.3 mmol) was added to a stirred solution of 4′-(4-fluoro-phenyl)-2′-methyl-[2,5′]bithiazolyl-4-carboxylic acid pentafluorophenyl ester (97 mg, 0.2 mmol), and DIEA (70 μL, 0.4 mmol) in DMF (1 mL), at room temperature. After 2 hrs the reaction was determined to be complete by HPLC analysis. The resulting crude mixture was purified by automated C18 reverse phase semi-preparative HPLC to afford the title compound (69 mg, 78% yield, mono TFA salt) as a tan solid. ¹H NMR (400 MHz, CDCl₃) δ 7.91 (s, 1H), 7.54 (m, 2H), 7.24 (m, 1H), 7.16 (m, 2H), 3.01 (d, 3H), 2.76 (s, 3H). LCMS: 333.7 (M+1)⁺.

### EXAMPLE 48

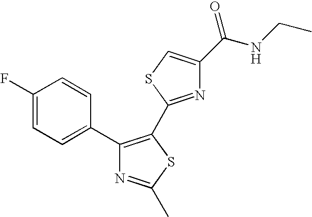

### 4′-(4-Fluoro-phenyl)-2′-methyl-|2,5′|bithiazolyl-4-carboxylic acid ethylamide

The title compound was prepared analogously to 4′-(4-fluoro-phenyl)-2′-methyl-[2,5′]bithiazolyl-4-carboxylic acid methylamide, where ethylamine was substituted for methylamine in step 4 of that sequence. ¹H NMR (400 MHz, CDCl₃) δ 7.91 (s, 1H), 7.54 (m, 2H), 7.22 (m, 1H), 7.16 (m, 2H), 3.49 (m, 2H), 2.77 (s, 1H), 1.26 (t, 3H). LCMS: 347.7 (M+1)⁺.

### EXAMPLE 49

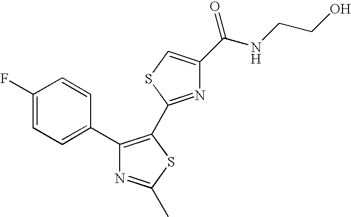

### 4′-(4-Fluoro-phenyl)-2′-methyl-|2,5′|bithiazolyl-4-carboxylic acid (2-hydroxy-ethyl)-amide

The title compound was prepared analogously to 4′-(4-fluoro-phenyl)-2′-methyl-[2,5′]bithiazolyl-4-carboxylic acid methylamide where ethanolamine was substituted for methylamine in step 4 of that sequence. ¹H NMR (400 MHz, CDCl₃) δ 7.93 (s, 1H), 7.63 (m, 1H), 7.54 (m, 2H), 7.16 (m, 2H), 3.84 (m, 2H), 3.63 (m, 2H), 2.76 (s, 3H). LCMS: 364.1 (M+1)⁺.

### EXAMPLE 50

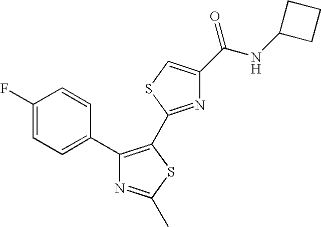

### 4′-(4-Fluoro-phenyl)-2′-methyl-|2,5′|bithiazolyl-4-carboxylic acid cyclobutylamide

The title compound was prepared analogously to 4′-(4-Fluoro-phenyl)-2′-methyl-[2,5′]bithiazolyl-4-carboxylic acid methylamide, where cyclobutylamine was substituted for methylamine in step 4 of that sequence. ¹H NMR (400 MHz, CDCl₃) δ 7.91 (s, 1H), 7.54 (m, 2H), 7.31 (m, 1H), 7.16 (m, 2H), 4.56 (m, 1H), 2.77 (s, 3H), 2.41 (m, 2H), 1.99 (m, 2H), 1.78 (m, 2H). LCMS: 373.6 (M+1)⁺.

### EXAMPLE 51

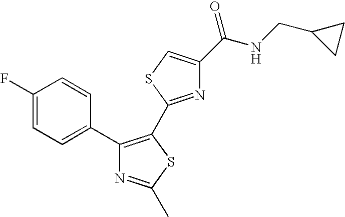

### 4′-(4-Fluoro-phenyl)-2′-methyl-|2,5′|bithiazolyl-4-carboxylic acid cyclopropylmethyl-amide

The title compound was prepared analogously to 4′-(4-fluoro-phenyl)-2′-methyl-[2,5′]bithiazolyl-4-carboxylic acid methylamide, where (aminomethyl)cyclopropane was substituted for methylamine in step 4 of that sequence. ¹H NMR (400 MHz, CDCl₃) δ 7.93 (s, 1H), 7.54 (m, 2H), 7.31 (m, 1H), 7.15 (m, 2H), 3.31 (m, 2H), 2.77 (s, 3H), 1.06 (m, 1H), 0.57 (m, 2H), 0.30 (m, 2H). LCMS: 373.7 (M+1)⁺.

### EXAMPLE 52

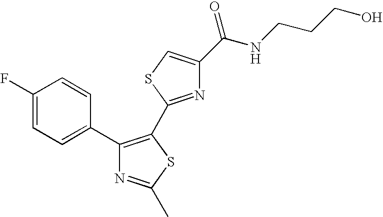

### 4′-(4-Fluoro-phenyl)-2′-methyl-|2,5′|bithiazolyl-4-carboxylic acid (3-hydroxy-propyl)-amide

The title compound was prepared analogously to 4′-(4-fluoro-phenyl)-2′-methyl-[2,5′]bithiazolyl-4-carboxylic acid methylamide, where 3-amino-1-propanol was substituted for methylamine in step 4 of that sequence. ¹H NMR (400 MHz, CDCl₃) δ7.93 (s, 1H), 7.54 (m, 3H), 7.15 (m, 2H), 3.68 (m, 2H), 3.62 (m, 2H), 2.76 (s, 3H), 1.81 (m, 2H). LCMS: 377.8 (M+1)⁺.

### EXAMPLE 53

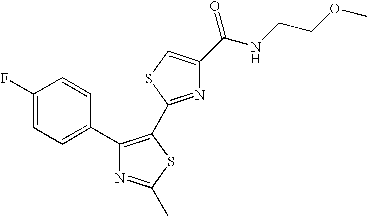

### 4′-(4-Fluoro-phenyl)-2′-methyl-|2,5′|bithiazolyl-4-carboxylic acid (2-methoxy-ethyl)-amide

The title compound was prepared analogously to 4′-(4-fluoro-phenyl)-2′-methyl-[2,5′]bithiazolyl-4-carboxylic acid methylamide, where 2-methoxyethanamine was substituted for methylamine in step 4 of that sequence. ¹H NMR (400 MHz, CDCl₃) δ 7.93 (s, 1H), 7.54 (m, 3H), 7.15 (m, 2H), 3.64 (m, 2H), 3.56 (m, 2H), 3.41 (s, 3H), 2.77 (s, 3H). LCMS: 377.7 (M+1)⁺.

### EXAMPLE 54

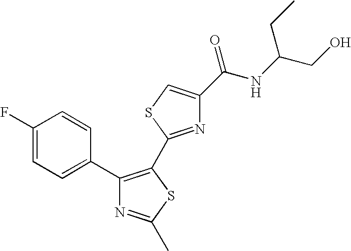

### 4′-(4-Fluoro-phenyl)-2′-methyl-|2,5′|bithiazolyl-4-carboxylic acid (1-hydroxymethyl-propyl)-amide

The title compound was prepared analogously to 4′-(4-fluoro-phenyl)-2′-methyl-[2,5′]bithiazolyl-4-carboxylic acid methylamide, where 2-amino-1-butanol was substituted for methylamine in step 4 of that sequence. ¹H NMR (400 MHz, CDCl₃) δ 7.95 (s, 1H), 7.54 (m, 2H), 7.35 (m, 1H), 7.15 (m, 2H), 4.02 (m, 1H), 3.82 (m, 1H), 3.71 (m, 1H), 2.76 (s, 3H), 1.73 (m, 1H), 1.64 (m, 1H), 1.01 (t, 3H). LCMS: 391.7 (M+1)⁺.

### EXAMPLE 55

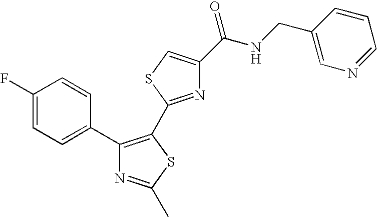

### 4′-(4-Fluoro-phenyl)-2′-methyl-|2,5′|bithiazolyl-4-carboxylic acid (pyridin-3-ylmethyl)-amide

The title compound was prepared analogously to 4′-(4-fluoro-phenyl)-2′-methyl-[2,5′]bithiazolyl-4-carboxylic acid methylamide, where 3-pyridinylmethanamine was substituted for methylamine in step 4 of that sequence. ¹H NMR (400 MHz, CDCl₃) δ 8.90 (m, 1H), 8.73 (m, 1H), 8.36 (d, 1H), 8.03 (dd, 1H), 7.96 (s, 1H), 7.79 (m, 1H), 7.52 (m, 2H), 7.15 (m, 2H), 4.78 (d, 2H), 2.75 (s, 3H). LCMS: 411.5 (M+1)⁺.

### EXAMPLE 56

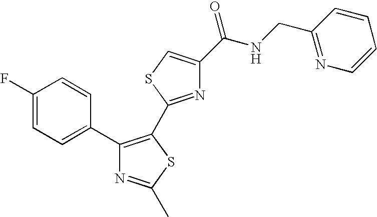

### 4′-(4-Fluoro-phenyl)-2′-methyl-|2,5′|bithiazolyl-4-carboxylic acid (pyridin-2-ylmethyl)-amide

The title compound was prepared analogously to 4′-(4-fluoro-phenyl)-2′-methyl-[2,5′]bithiazolyl-4-carboxylic acid methylamide, where 2-pyridinylmethanamine was substituted for methylamine in step 4 of that sequence. ¹H NMR (400 MHz, CDCl₃) δ 8.94 (m, 1H), 8.74 (m, 1H), 8.20 (dd, 1H), 7.94 (d, 1H), 7.87 (s, 1H), 7.68 (m, 1H), 7.52 (m, 2H), 7.15 (m, 2H), 4.96 (d, 2H), 2.77 (s, 3H). LCMS: 411.5 (M+1)⁺.

### EXAMPLE 57

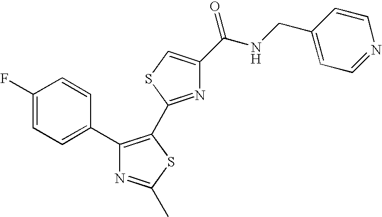

### 4′-(4-Fluoro-phenyl)-2′-methyl-|2,5′|bithiazolyl-4-carboxylic acid (pyridin-4-ylmethyl)-amide

The title compound was prepared analogously to 4′-(4-fluoro-phenyl)-2′-methyl-[2,5′]bithiazolyl-4-carboxylic acid methylamide, where 4-pyridinylmethanamine was substituted for methylamine in step 4 of that sequence. ¹H NMR (400 MHz, CDCl₃) δ 9.15 (m, 1H), 8.74 (d, 2H), 8.20 (s, 1H), 7.73 (d, 2H), 7.63 (m, 2H), 7.35 (m, 2H), 4.63 (d, 2H), 2.73 (d, 3H). LCMS: 410.9 (M+1)⁺.

### EXAMPLE 58

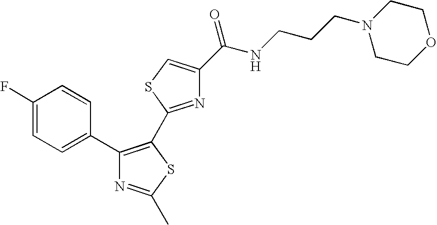

### 4′-(4-Fluoro-phenyl)-2′-methyl-|2,5′|bithiazolyl-4-carboxylic acid (3-morpholin-4-yl-propyl)-amide

The title compound was prepared analogously to 4′-(4-fluoro-phenyl)-2′-methyl-[2,5′]bithiazolyl-4-carboxylic acid methylamide, where 3-(4-morpholinyl)-1-propanamine was substituted for methylamine in step 4 of that sequence. ¹H NMR (400 MHz, CDCl₃) δ 7.89 (s, 1H), 7.63 (m, 1H), 7.53 (m, 2H), 7.16 (m, 2H), 3.98 (m, 4H), 3.54 (m, 4H), 3.14 (m, 2H), 2.88 (m, 2H), 2.77 (s, 3H), 2.15 (m, 2H). LCMS: 447.5 (M+1)⁺.

### EXAMPLE 59

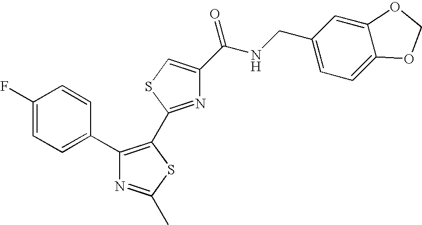

### 4′-(4-Fluoro-phenyl)-2′-methyl-|2,5′|bithiazolyl-4-carboxylic acid (benzo|1,3|dioxol-5-ylmethyl)-amide

The title compound was prepared analogously to 4′-(4-fluoro-phenyl)-2′-methyl-[2,5′]bithiazolyl-4-carboxylic acid methylamide, where 1,3-benzodioxol-5-ylmethananimine was substituted for methylamine in step 4 of that sequence. ¹H NMR (400 MHz, CDCl₃) δ 7.96 (s, 1H), 7.53 (m, 3H), 7.12 (m, 2H), 6.85 (m, 1H), 6.80 (m, 2H), 5.95 (s, 2H), 4.55 (m, 2H), 2.74 (s, 3H). LCMS: 453.9 (M+1)⁺.

### EXAMPLE 60

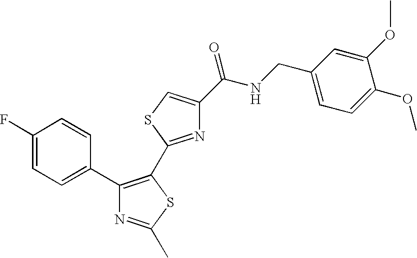

### 4′-(4-Fluoro-phenyl)-2′-methyl-|2,5′|bithiazolyl-4-carboxylic acid 3,4-dimethoxy-benzylamide

The title compound was prepared analogously to 4′-(4-fluoro-phenyl)-2′-methyl-[2,5′]bithiazolyl-4-carboxylic acid methylamide, where (3,4-dimethoxyphenyl)methanamine was substituted for methylamine in step 4 of that sequence. ¹H NMR (400 MHz, CDCl₃) δ 7.96 (s, 1H), 7.53 (m, 3H), 7.12 (m, 2H), 6.85 (m, 3H), 4.57 (d, 2H), 3.88 (s, 6H), 2.75 (s, 3H). LCMS: 469.9 (M+1)⁺.

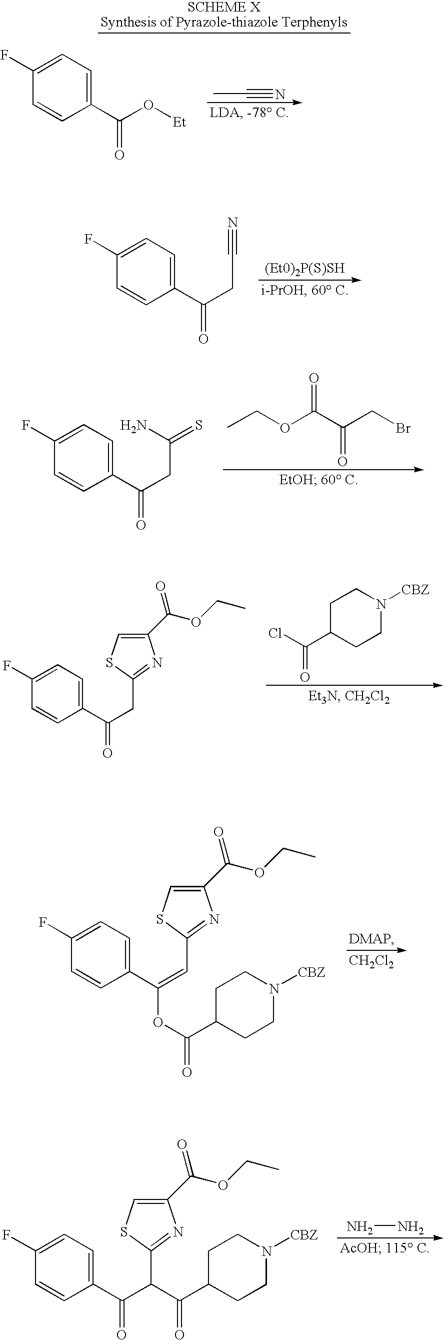

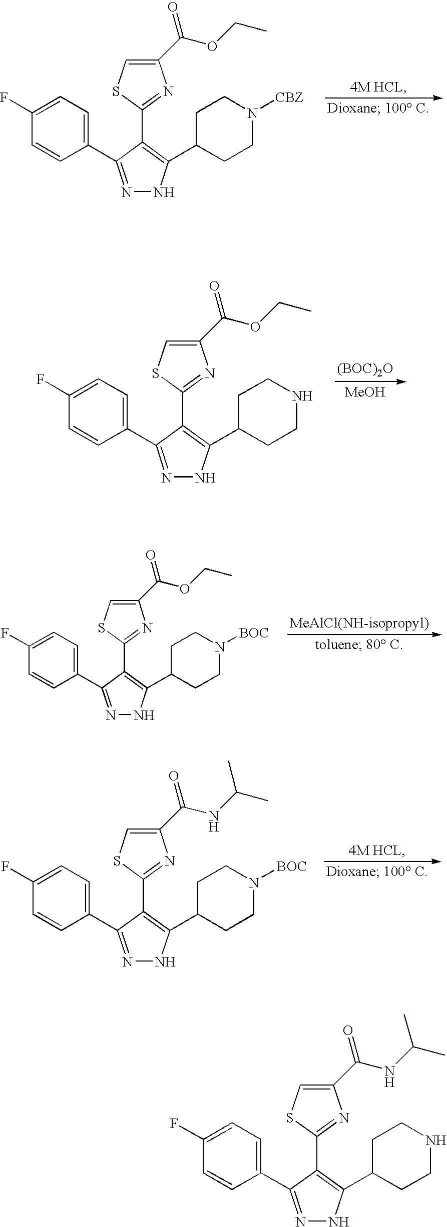

### EXAMPLE 61

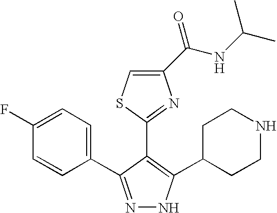

### 2-|3-(4-Fluoro-phenyl)-5-piperidin-4-yl-1H-pyrazol-4-yl|-thiazole-4-carboxylic acid isopropylamide

Step 1:

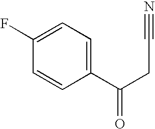

### 3-(4-Fluoro-phenyl)-3-oxo-propionitrile

A 500 mL 3-neck flask was charged with diisopropylamine (15.5 mL, 110 mmol) and THF (0.300 mL). The resultant solution was cooled to −78° C., where butyllithium (62.5 mL, 100 mmol) was added. After 10 minutes, a solution of acetonitrile (5.2 mL, 100 mmol) in THF (10 mL) was added and the reaction was stirred for 15 minutes at −78° C. Next, a solution of -fluoro-benzoic acid ethyl ester (13.2 mL, 90 mmol) in THF (26 mL) was added to the flask, and the reaction stirred at room temperature for 30 minutes. Work-up: the reaction mixture was concentrated, diluted in EtOAc (150 mL), and washed with H₂O (150 mL). The aqueous layer was acidified with 4N HCl to pH=6, extracted twice with EtOAc. The organics were dried with MgSO₄, concentrated, triturated with Et₂O, sonicated, and filtered, giving a white crystalline product (72% yield).
Step 2:

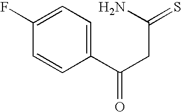

### 3-(4-Fluoro-phenyl)-3-oxo-thiopropionamide

A 300 mL round bottom flask was charged with 3-(4-Fluoro-phenyl)-3-oxo-propionitrile (10 g, 61.0 mmol), isopropanol (123 mL) and dithiophosphoric acid 0,0-diethyl ester (31 mL, 183.0 mmol). The reaction was stirred for 4 hours at 58° C. Reaction progress was monitored by TLC (100% DCM). Work-up: the crude reaction was concentrated, triturated with DCM, sonicated, and filtered, giving 7.6 g (56% yield) of a tan solid. LCMS: 198.20 (M+1)⁺.
Step 3:

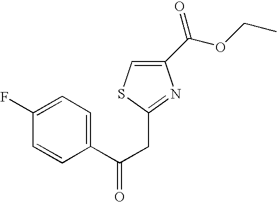

### 2-|2-(4-Fluoro-phenyl)-2-oxo-ethyl|-thiazole-4-carboxylic acid ethyl ester

A 250 mL round bottom flask was charged with 3-(4-Fluoro-phenyl)-3-oxo-thiopropionamide (5 g, 25.4 mmol), EtOH (75 mL), and ethyl bromopyruvate (3.83 mL, 30.5 mmol). The resulting mixture was stirred for 2 hours at 60° C. Reaction progress was monitored by TLC (100% DCM). Work-up: the crude reaction was diluted with water and extracted with EtOAc (3×100 mL). The resulting organics were washed with brine, dried over MgSO₄, and concentrated to a solid. The solid was suspended in ether, sonicated, and filtered; resulting in 6.3 g of bright yellow solid product (83% yield). LCMS: 293.93 (M+1)⁺.
Step 4:

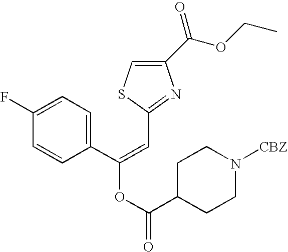

### Piperidine-1,4-dicarboxylic acid 1-benzyl ester 4-|2-(4-ethoxycarbonyl-thiazol-2-yl)-1-(4-fluoro-phenyl)-vinyl|ester

A 250 mL flask was charged with 2-[2-(4-Fluoro-phenyl)-2-oxo-ethyl]-thiazole-4-carboxylic acid ethyl ester (4 g, 13.6 mmol); DCM (50 mL), and Et₃N (5.3 mL, 38.1 mmol). 4-chlorocarbonyl-piperidine-1-carboxylic acid benzyl ester (5.1 g, 18.3 mmol) was added to the solution, and the reaction was stirred for 30 minutes at room temperature, where it was monitored by LCMS and TLC (EtOAc/Hex=1:1). The crude reaction was diluted with EtOAc (200 mL), washed with water, dried over MgSO₄, concentrated to a solid; giving 35.9 g of product (100% yield). LCMS: 539.03 (M+1)⁺.
Step 5

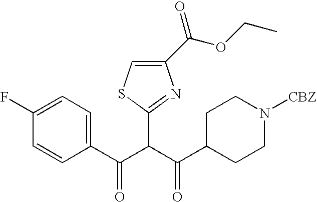

### 4-|2-(4-Ethoxycarbonyl-thiazol-2-yl)-3-(4-fluoro-phenyl)-3-oxo-propionyl|-piperidine-1-carboxylic acid benzyl ester

A 500 mL round bottom flask was charged with Piperidine-1,4-dicarboxylic acid 1-benzyl ester 4-[2-(4-ethoxycarbonyl-thiazol-2-yl)-1-(4-fluoro-phenyl)-vinyl]ester (35.9 g, 67 mmol), DCM (200 mL), and DMAP (16.3 g, 134 mmol). The reaction was stirred at room temperature for 4 hours. Reaction progress was monitored by LC/MS and TLC (EtOAc/Hex=1:1). Workup: reaction mixture was diluted DCM, washed with 1 M HCl (100 mL), dried over MgSO₄; and concentrated. The crude material was purified via flash chromatography, eluted with 100% DCM to 50% EtOAc/DCM, giving 25 g (70% yield) of product, as a yellow oil. LCMS: 539.44 (M+1)⁺.
Step 6:

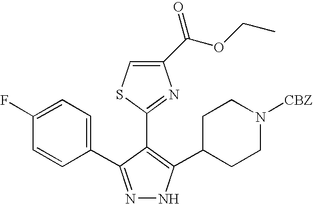

### 4-|4-(4-Ethoxycarbonyl-thiazol-2-yl)-5-(4-fluoro-phenyl)-2H-pyrazol-3-yl|-piperidine-1-carboxylic acid benzyl ester

A 50 mL flask was charged with 4-[2-(4-Ethoxycarbonyl-thiazol-2-yl)-3-(4-fluoro-phenyl)-3-oxo-propionyl]-piperidine-1-carboxylic acid benzyl ester (535 mg, 1.0 mmol), acetic acid (4.0 mL), and hydrazine (0.157 mL, 5.0 mmol). The reaction was stirred for 2 hours at 115° C. Reaction progress was monitored by LC/MS and TLC (EtOAc/Hex=1:1). Workup: the reaction was concentrated, diluted with EtOAc, washed with sodium bicarbonate(aq.), brine, dried over MgSO₄, and concentrated to a brown oil. The oil was purified via flash chromatography (15% ACN/DCM-40% ACN/DCM), giving 480 mg (90% yield) of a yellow solid. LCMS: 535.21 (M+1)⁺.
Step 7

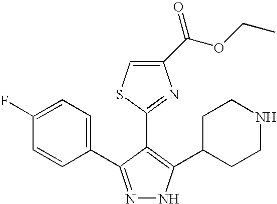

### 2-|3-(4-Fluoro-phenyl)-5-piperidin-4-yl-1H-pyrazol-4-yl|-thiazole-4-carboxylic acid ethyl ester

A 500 mL Teflon capped sealed tube was charged with 4-[4-(4-Ethoxycarbonyl-thiazol-2-yl)-5-(4-fluoro-phenyl)-2H-pyrazol-3-yl]-piperidine-1-carboxylic acid benzyl ester (12 g, 22.5 mmol), and 4 M HCl in dioxane (225 mL). The reaction was stirred for 1 hour at 100° C. Reaction progress was monitored by LC/MS. Work-up: reaction was cooled to room temperature, and concentrated to a yellow solid; (9 g, 92% yield). LCMS: 402.92 (M+1)⁺.
Step 8:

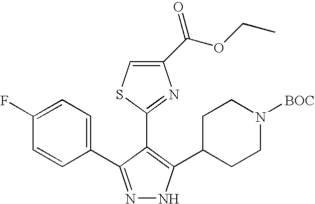

4-|4-(4-Ethoxycarbonyl-thiazol-2-yl)-5-(4-fluoro-phenyl)-2H-pyrazol-3-yl|piperidine-1-carboxylic acid tert-butyl ester

A 500 mL flask was charged with the 2-[3-(4-Fluoro-phenyl)-5-piperidin-4-yl-1H-pyrazol-4-yl]-thiazole-4-carboxylic acid ethyl ester HCl-salt (9 g, 21 mmol), Et₃N (15.6 mL, 113.0 mmol), and mEtOH (75 mL). Di-t-Butyl Dicarbonate (5.87 g, 27 mmol) was added to the solution, and the reaction was stirred at room temperature for 1 hour. Reaction progress was monitored by LC/MS and TLC (EtOAc/Hex=1:1). The mixture was diluted in EtOAc (300 mL), washed with water, dried over MgSO₄, and concentrated to a white solid (9 g, 87% yield). LCMS: 501.61 (M+1)⁺.
Step 9:

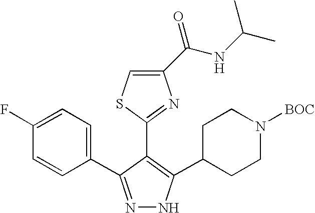

### 4-|5-(4-Fluoro-phenyl)-4-(4-isopropylcarbamoyl-thiazol-2-yl)-2H-pyrazol-3-yl|-piperidine-1-carboxylic acid tert-butyl ester

A 250 mL round bottom flask was charged with 4-[4-(4-Ethoxycarbonyl-thiazol-2-yl)-5-(4-fluoro-phenyl)-2H-pyrazol-3-yl]piperidine-1-carboxylic acid tert-butyl ester (5.0 g, 10 mmol), toluene (50.0 mL), and MeAlCl(NH-isopropyl) (30 mL, 0.67M, prepared as described in Synthetic Communications, 12 (13), 989-993 (1982)). The resulting solution was stirred at 80° C. for 1 hr. Reaction progress was monitored by LC/MS. Work-up: the reaction was diluted with DCM (800 mL), poured over Na₂SO₄-10H₂O (210 g), and stirred at room temperature for 1 hr. The crude solution was dried with MgSO₄, filtered, and concentrated to a solid. The solid was triturated with EtOAc, sonicated, and filtered; giving 5.05 g of product (98% yield). LCMS: 514.48 (M+1)⁺.
Step 10:

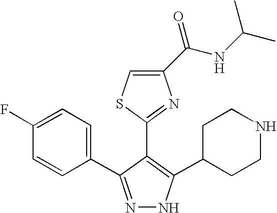

### 2-|3-(4-Fluoro-phenyl)-5-piperidin-4-yl-1H-pyrazol-4-yl|-thiazole-4-carboxylic acid isopropylamide

A Teflon capped sealed tube was charged with 4-[5-(4-Fluoro-phenyl)-4-(4-isopropyl-carbamoyl-thiazol-2-yl)-2H-pyrazol-3-yl]-piperidine-1-carboxylic acid tert-butyl ester (2.5 μg, 4.9 mmol), MeOH (6 mL), and 4M HCL in dioxane (14 mL). The mixture was stirred for 1 hour at 50° C. Reaction progress was monitored by LC/MS. Work-up: the mixture was concentrated to a solid to yield 2.06 g (94% yield) and was taken to the next step without further purification. 50 mg of the crude product was purified via reverse phase HPLC, and yielded 27% (calculated as 2×TFA-salt). ¹H NMR (400 MHz, CDCl₃) δ 10.05 (bs, 1H), 9.12 (bs, 1H), 7.99 (s, 1H), 7.35 (m, 2H), 7.17 (m, 2H), 7.02 (t, 2H), 6.88 (d, 1H), 4.18 (m, 1H), 3.59 (d, 2H), 3.41 (m, 2H), 3.08 (m, 5H), 2.23 (m, 4H), 2.19 (m, 2H), 1.18 (d, 6H). LCMS: 414.97 (M+1)⁺.

### EXAMPLE 62

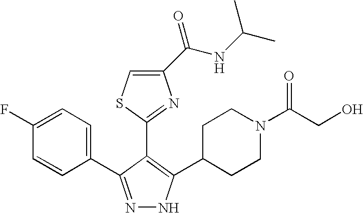

### 2-{3-(4-Fluoro-phenyl)-5-|1-(2-hydroxy-acetyl)-piperidin-4-yl|-1H-pyrazol-4-yl}thiazole-4-carboxylic acid isopropylamide

A 100 mL flask was charged with 2-[3-(4-Fluoro-phenyl)-5-piperidin-4-yl-1H-pyrazol-4-yl]-thiazole-4-carboxylic acid isopropylamide (80 mg, 0.2 mmol, prepared as described in step 10 of Example 61), DCM (0.7 mL), Et₃N (0.140 mL, 1.0 mmol), and acetoxy acetyl hydrochloride (33.0 μL, 0.30 mmol). The reaction was stirred for 15 minutes at room temperature, and reaction progress was monitored by LC/MS. Work-up: the crude mixture was diluted with EtOAc, washed with water, dried over MgSO₄, and concentrated to yield a solid. The solid was dissolved in a 1:1 solution of THF/MeOH (0.30 mL), and treated with LiOH (0.60 mL, 0.6 mmol). The solution was stirred at 50° C. for 30 minutes. Workup: the reaction was diluted in EtOAc, washed with water, dried over MgSO₄, and concentrated to a solid. The material was purified via HPLC to yield 41.8 mg of product (59% yield, calculated as TFA salt). ¹H NMR (400 MHz, CDCl₃): δ 8.87 (bs, 1H), 7.99 (s, 1H), 7.35 (m, 2H), 7.17 (m, 2H), 7.02 (t, 2H), 6.88 (d, 1H), 4.18 (m, 1H), 3.59 (d, 2H), 3.41 (m, 3H), 3.08 (m, 2H), 2.23 (m, 4H), 2.19 (m, 2H), 1.18 (d, 6H). LCMS: 472.23 (M+1)⁺.

### EXAMPLE 63

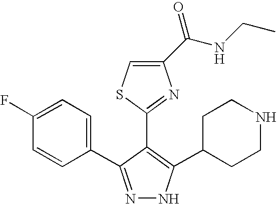

### 2-|3-(4-Fluoro-phenyl)-5-piperidin-4-yl-1H-pyrazol-4-yl|-thiazole-4-carboxylic acid ethylamide

Step 1:

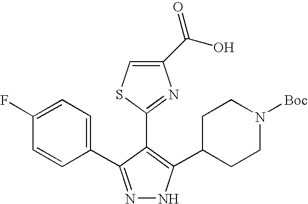

### 4-|4-(4-Carboxy-thiazol-2-yl)-5-(4-fluorophenyl)-2H-pyrazol-3-yl|-piperidine-1 carboxylic acid tert-butyl ester

A 250 mL round bottom flask was charged with 4-[4-(4-Ethoxycarbonyl-thiazol-2-yl)-5-(4-fluoro-phenyl)-2H-pyrazol-3-yl]-piperidine-1-carboxylic acid tert-butyl ester (2.6 g, 5.2 mmol, prepared as described in step 8 of Example 61), and a 1:1 solution of THF/MeOH (20.0 mL). An aqueous solution of LiOH, 1 M (12.0 mL, 12.0 mmol) was added, and the reaction was stirred at 50° C. for 1 hour. The progress was monitored by LC/MS. Work-up: the reaction was diluted with EtOAc, washed with water, dried over MgSO₄, and concentrated to a solid (2.58 g, 100%). LCMS: 473.46 (M+1)⁺.
Step 2:

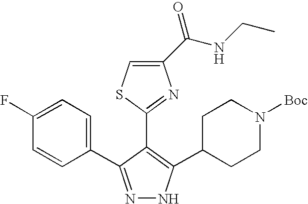

### 4-|4-(4-Ethylcarbamoyl-thiazol-2-yl)-5-(4-fluoro-phenyl)-2H-pyrazol-3-yl|-piperidine-1-carboxylic acid tert-butyl ester

A 100 mL flask was charged with 2M ethylamine in dioxane (0.465 mL, 0.8 mmol), DMF (1.76 mL), Et₃N (0.143 mL; 1.06 mmol), and 4-[4-(4-Carboxy-thiazol-2-yl)-5-(4-fluoro-phenyl)-2H-pyrazol-3-yl]-piperidine-1 carboxylic acid tert-butyl ester (250 mg, 0.53 mmol). The solution was stirred for a few minutes, and HATU was added (201 mg, 0.53 mmol) to the flask. The reaction was stirred 1 hour at room temperature, and the progress was monitored by LC/MS. Work-up: the crude mixture was the reaction was diluted with EtOAc; washed with 1 M HCl (aq), water, and brine. The solution was dried over MgSO₄, and concentrated to a solid (2.58 g, 100% yield). LCMS: 500.50 (M+1)⁺.
Step 3:

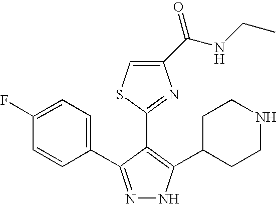

### 2-|3-(4-Fluoro-phenyl)-5-piperidin-4-yl-1H-pyrazol-4-yl|-thiazole-4-carboxylic acid ethylamide

A flask was charged with 4-[4-(4-Ethylcarbamoyl-thiazol-2-yl)-5-(4-fluoro-phenyl)-2H-pyrazol-3-yl]-piperidine-1-carboxylic acid tert-butyl ester (258 mg, 0.516 mmol), and a solution of 30% TFA/DCM (6 mL). The reaction was stirred for 20 minutes at room temperature, and the progress was monitored by TLC. Work-up: the solution was diluted with toluene (20 mL), and concentrated to dryness. The resultant material was purified via reverse phase HPLC, and yielded 133 mg of transparent, tan crystals (63%, calculated as bis-TFA salt). ¹H NMR (400 MHz, CDCl₃) δ: 8.00 (s, 1H), 7.36 (m, 2H), 7.17 (m, 2H), 7.02 (t, 2H), 6.92 (s, 1H), 3.59 (m, 2H), 3.43 (m, 2H), 3.08 (m, 2H), 2.24 (m, 1H), 1.73 (m, 2H), 1.18 (t, 3H), 0.80 (bs, 1H). LCMS: 400.75 (M+1)⁺.

### EXAMPLE 64

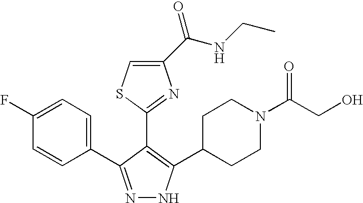

### 2-{3-(4-Fluoro-phenyl)-5-|1-(2-hydroxy-acetyl)-piperidin-4-yl|-1H-pyrazol-4-yl}-thiazole-4-carboxylic acid ethylamide

The title compound was prepared analogously to Example 62. ¹H NMR (400 MHz, CDCl₃) δ: 7.96 (s, 1H), 7.35 (m, 2H), 7.19 (m, 2H), 7.07 (m, 2H), 4.62 (m, 1H), 4.15 (d, 2H), 3.59 (m, 1H), 3.39 (m, 4H), 3.04 (m, 2H), 2.76 (m, 3H), 2.06 (m, 2H), 1.78 (m, 2H), 1.18 (t, 3H), 0.80 (bs, 1H). LCMS: 458.32 (M+1)⁺.

### EXAMPLE 65

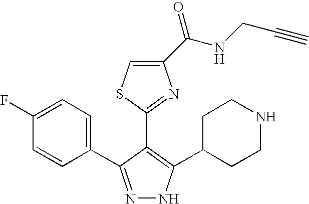

### 2-|3-(4-Fluoro-phenyl)-5-piperidin-4-yl-1H-pyrazol-4-yl|-thiazole-4-carboxylic acid prop-2-ynylamide

The title compound was prepared analogously to Example 63, (2-[3-(4-Fluoro-phenyl)-5-piperidin-4-yl-1H-pyrazol-4-yl]-thiazole-4-carboxylic acid ethylamide). ¹H NMR (400 MHz, CDCl₃) δ 8.04 (s, 1H), 7.51 (m, 2H), 7.24 (m, 1H), 7.14 (m, 2H), 4.26 (t, 2H), 3.68 (m, 4H), 3.21 (m, 2H), 2.51 (d, 1H), 2.37 (m, 2H), 1.74 (m, 1H), 1.25 (s, 1H), 0.88 (bs, 1H). LCMS: 410.65 (M+1)⁺.

### EXAMPLE 66

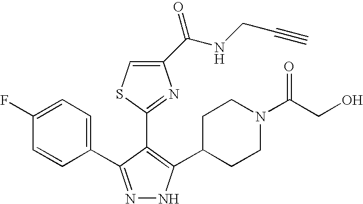

### 2-{3-(4-Fluoro-phenyl)-5-|1-(2-hydroxy-acetyl)-piperidin-4-yl|-1H-pyrazol-4-yl}-thiazole-4-carboxylic acid prop-2-ynylamide

The title compound was prepared analogously to Example 62, ((2-{3-(4-Fluoro-phenyl)-5-[1-(2-hydroxy-acetyl)-piperidin-4-yl]-1H-pyrazol-4-yl}-thiazole-4-carboxylic acid isopropylamide). ¹H NMR (400 MHz, CDCl₃) δ 8.04 (s, 1H), 7.44 (m, 3H), 7.24 (m, 1H), 7.15 (t, 3H), 4.74 (d, 1H), 4.25 (d, 2H), 4.21 (d, 3H), 3.68 (d, 1H), 3.49 (s, 1H), 3.47 (m, 1H), 3.13 (m, 2H), 2.89 (m, 2H), 2.32 (m, 2H), 2.14 (m, 3H), 1.85 (m, 2H), 0.80 (bs, 1H)). LCMS: 468.31 (M+1)⁺.

### EXAMPLE 67

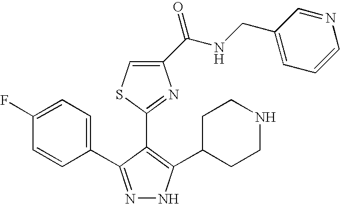

### 2-|3-(4-Fluoro-phenyl)-5-piperidin-4-yl-1H-pyrazol-4-yl|-thiazole-4-carboxylic acid (pyridin-3-ylmethyl)-amide

The title compound was prepared analogously to Example 63, (2-[3-(4-Fluoro-phenyl)-5-piperidin-4-yl-1H-pyrazol-4-yl]-thiazole-4-carboxylic acid ethylamide). ¹H NMR (400 MHz, DMSO-d₆) δ: 8.89 (m, 1H), 8.6 (dd, 2H)), 8.34 (m, 1H), 7.45 (m, 2H), 7.35 (m, 1H), 4.45 (d, 2H), 3.59 (m, 2H), 3.43 (m, 4H), 2.98 (m, 2H), 2.87 (bs, 1H), 2.14 (m, 2H), 1.89 (m, 2H). LCMS: 463.35 (M+1)⁺.

### EXAMPLE 68

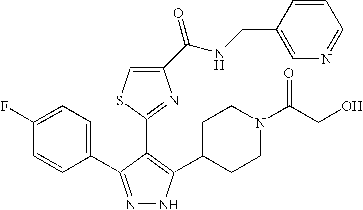

### 2-{3-(4-Fluoro-phenyl)-5-|1-(2-hydroxy-acetyl)-piperidin-4-yl|-1H-pyrazol-4-yl}thiazole-4-carboxylic acid (pyridin-3-ylmethyl)-amide

The title compound was prepared analogously to Example 62, ((2-{3-(4-Fluoro-phenyl)-5-[1-(2-hydroxy-acetyl)-piperidin-4-yl]-1H-pyrazol-4-yl}-thiazole-4-carboxylic acid isopropylamide). ¹H NMR (400 MHz, DMSO-d₆) δ: 8.82 (m, 1H), 8.62 (s, 1H)), 8.55 (d, 1H), 8.28 (s, 1H), 7.94 (d, 1H), 7.51 (m, 3H), 7.23 (m, 1H), 4.55 (d, 2H), 4.11 (d, 2H), 3.43 (m, 4H), 2.96 (m, 2H), 2.62 (m, 1H), 1.89 (m, 2H). LCMS: 521.53 (M+1)⁺.

### EXAMPLE 69

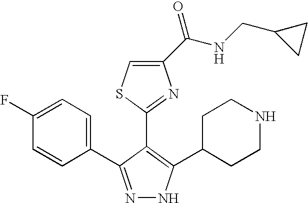

### 2-|3-(4-Fluoro-phenyl)-5-piperidin-4-yl-1H-pyrazol-4-yl|-thiazole-4-carboxylic acid cyclopropylmethyl-amide

The title compound was prepared analogously to Example 63, (2-[3-(4-Fluoro-phenyl)-5-piperidin-4-yl-1H-pyrazol-4-yl]-thiazole-4-carboxylic acid ethylamide). ¹H NMR (400 MHz, DMSO-d₆) δ: 13.38 (bs, 1H) 8.84 (bs, 1H), 8.58 (s, 1H)), 8.38 (m, 1H), 8.14 (s, 1H), 7.54 (d, 2H), 7.36 (d, 2H), 7.23, 5.76 (s, 1H), 3.53 (m, 1H), 3.12 (m, 6H), 2.12 (d, 2H), 1.89 (d, 2H), 1.14 (m, 5H), (0.3 (dd, 4). LCMS: 426.48 (M+1)⁺.

### EXAMPLE 70

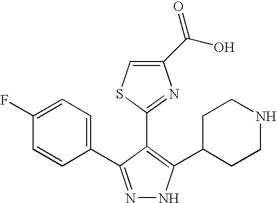

### 2-(3-(4-fluorophenyl)-5-(piperidin-4-yl)-1H-pyrazol-4-yl)thiazole-4-carboxylic acid (DS308-030)

A 50 mL flask was charged with 2-(5-(1-(tert-butoxycarbonyl)piperidin-4-yl)-3-(4-fluorophenyl)-1H-pyrazol-4-yl)thiazole-4-carboxylic acid (47.3 mg, 0.1 mmol, described in Example 63), and a solution of 30% TFA-DCM (0.30 mL). The reaction was stirred for 20 minutes at room temperature; and was monitored by LC/MS. Work-up: the solution was diluted with toluene (20 mL), and concentrated to dryness. The resulting material was purified via reverse phase HPLC, and yielded 39.2 mg of transparent, tan crystals (100%, calculated as bis-TFA salt). ¹H NMR (400 MHz, DMSO-d₆) δ: 13.34 (bs, 1H), 13.01 (bs, 1H), 8.52 (bs, 1H), 8.32 (s, 1H), 7.50 (m, 2H), 7.32 (d, 2H), 5.74 (s, 3H), 4.18 (bs, 1H), 3.36 (d, 2H), 3.18 (bs, 1H), 2.98 (bs, 3H), 2.10 (d, 2H), 1.95 (m, 3H), 1.1 (bs, 1H). LCMS: 373.17 (M+1)⁺.

### EXAMPLE 71

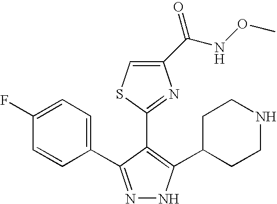

### 2-|3-(4-Fluoro-phenyl)-5-piperidin-4-yl-1H-pyrazol-4-yl|-thiazole-4-carboxylic acid methoxy-amide

Step 1:

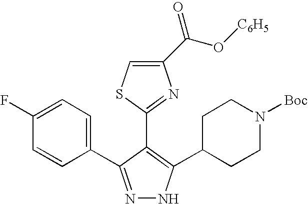

### 4-|5-(4-Fluoro-phenyl)-4-(4-pentafluorophenyloxycarbonyl-thiazol-2-yl)-2H-pyrazol-3-yl|-piperidine-1-carboxylic acid tert-butyl ester

A 100 mL flask was charged with 2-(5-(1-(tert-butoxycarbonyl)piperidin-4-yl)-3-(4-fluorophenyl)-1H-pyrazol-4-yl)thiazole-4-carboxylic acid (1.0 g, 2.1 mmol), pyridine (0.20 mL, 2.54 mmol), DMF (7.0 mL), and trifluoro-acetic acid pentafluorophenyl ester (0.472 mL, 2.75 mmol). The reaction was stirred at room temperature for 20 minutes, and the progress was monitored by LC/MS. Work-up: the crude reaction was concentrated and purified via chromatography (0-40% ACN/DCM) to give 0.89 g of a white solid (67% yield). LCMS: 639.42 (M+1)⁺.
Step 2:

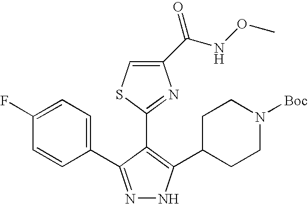

### 4-|5-(4-Fluoro-phenyl)-4-(4-methoxycarbamoyl-thiazol-2-yl)-2H-pyrazol-3-yl|-piperidine-1-carboxylic acid tert-butyl ester

A 50 mL flask was charged with 4-[5-(4-Fluoro-phenyl)-4-(4-pentafluoro-phenyloxycarbonyl-thiazol-2-yl)-2H-pyrazol-3-yl]-piperidine-1-carboxylic acid tert-butyl ester (100 mg, 0.156 mmol), Et3N (87.2 ul, 0.624 mmol), DCM (0.52 mL), and methoxyamine HCl (20 mg, 0.234 mmol). The reaction was stirred at room 55 degC. for 20 minutes, and the progress was monitored by LC/MS. Work-up: the crude mixture was the reaction was diluted with EtOAc; washed with 1 M HCl (aq), water, and brine. The solution was dried over MgSO4, and concentrated to a solid. The crude solid was taken to the next step without purification to give 78 mg of product (100% yield). However, for Examples where this was a final step in a sequence (no further deprotection), the crude material was purified via reverse phase HPLC, and isolated as a bis-TFA salt). LCMS: 502.45 (M+1)⁺.
Step 3:

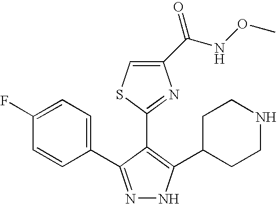

### 2-|3-(4-Fluoro-phenyl)-5-piperidin-4-yl-1H-pyrazol-4-yl|-thiazole-4-carboxylic acid methoxy-amide

A flask was charged with 4-[5-(4-Fluoro-phenyl)-4-(4-methoxycarbamoyl-thiazol-2-yl)-2H-pyrazol-3-yl]-piperidine-1-carboxylic acid tert-butyl ester (78.0 mg, 0.156 mmol), and a solution of 30% TFA/DCM (1.52 mL). The reaction was stirred for 20 minutes at room temperature, and the progress was monitored by TLC. Work-up: the solution was diluted with toluene (20 mL), and concentrated to dryness. The resulting material was purified via reverse phase HPLC, giving 51.1 mg of transparent tan crystals (81.0% yield, calculated as a Bis-TFA salt). ¹H NMR (400 MHz, DMSO-d₆) δ: 13.34 (bs, 1H), 11.53 (s, 1H), 8.52 (bs, 1H), 8.32 (bs, 1H), 8.21 (m, 1H), 7.51 (m, 2H), 7.34 (d, 2H), 7.25 (bs, 1H), 3.71 (s, 3H), 3.55 (bs, 1H), 2.04 (d, 2H), 1.87 (m, 3H), LCMS: 402.459 (M+1)⁺.

### EXAMPLE 72

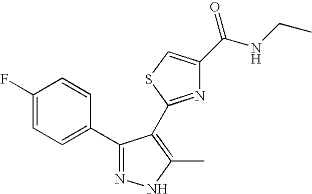

### 2-|3-(4-Fluoro-phenyl)-5-methyl-1H-pyrazol-4-yl|-thiazole-4-carboxylic acid ethylamide

The title compound was prepared analogously to Example 71, where 2-[3-(4-Fluoro-phenyl)-5-methyl-1H-pyrazol-4-yl]-thiazole-4-carboxylic acid pentafluorophenyl ester, was substituted for 4-[5-(4-Fluoro-phenyl)-4-(4 pentafluorophenyloxy-carbonyl-thiazol-2-yl)-2H-pyrazol-3-yl]-piperidine-1-carboxylic acid tert-butyl ester, and ethylamine was substituted for methoxyamine HCl in the final step of that example. ¹H NMR (400 MHz, DMSO-d₆) δ: 13.18 (s, 1H), 8.10 (bs, 1H), 7.50 (m, 2H), 7.22 (m, 2H), 3.31 (s, 3H), 3.28 (m, 2H), 2.42 (bs, 1H), 1.08 (m, 3H). LCMS: 330.96 (M+1)⁺.

### EXAMPLE 73

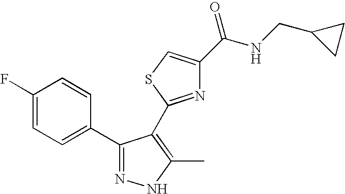

### 2-|3-(4-Fluoro-phenyl)-5-methyl-1H-pyrazol-4-yl|-thiazole-4-carboxylic acid cyclopropylmethyl-amide

The title compound was prepared analogously to Example 72. ¹H NMR (400 MHz, DMSO-d₆) δ: 13.20 (s, 1H), 8.12 (s, 1H), 8.08 (bs, 1H), 7.53 (m, 2H), 7.24 (m, 2H), 3.32 (s, 3H), 3.14 (m, 2H), 2.44 (s, 1H), 1.08 (m, 3H), 0.41 (d, 1H)). LCMS: 357.0 (M+1)⁺.

### EXAMPLE 74

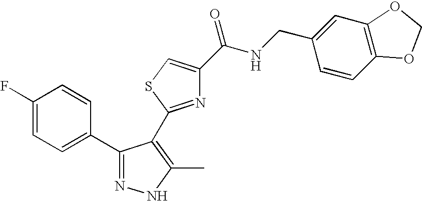

### 2-|3-(4-Fluoro-phenyl)-5-methyl-1H-pyrazol-4-yl|-thiazole-4-carboxylic acid (benzo|1,3|dioxol-5-yl methyl)-amide

The title compound was prepared analogously to Example 72. ¹H NMR (400 MHz, DMSO-d₆) δ: 13.19 (s, 1H), 8.61 (bs, 1H), 8.15 (s, 1H), 7.53 (m, 2H), 7.20 (m, 2H), 6.88 (dd, 2H), 6.79 (d, 1H), 6.02 (s, 2H), 4.37 (d, 2H), 3.32 (s, 3H), 2.50 (bs, 1H). LCMS: 437.4 (M+1)⁺.

### EXAMPLE 75

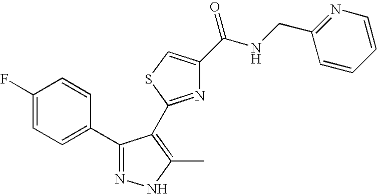

### 2-|3-(4-Fluoro-phenyl)-5-methyl-1H-pyrazol-4-yl|-thiazole-4-carboxylic acid (pyridin-2-ylmethyl)-amide

The title compound was prepared analogously to Example 72. ¹H NMR (400 MHz, DMSO-d₆) δ: 13.22 (s, 1H), 8.79 (bs, 1H), 8.51 (s, 1H), 8.16 (s, 1H), 7.76 (m, 1H), 7.55 (m, 2H), 7.29 (m, 4H), 4.59 (d, 2H), 3.59 (bs, 1H), 3.32 (s, 3H), 2.53 (s, 1H), 1.78 (bs, 1H). LCMS: 394.46 (M+1)⁺.

### EXAMPLE 76

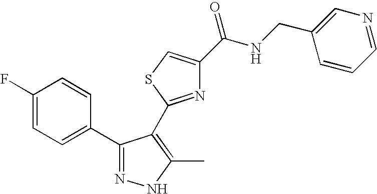

### 2-|3-(4-Fluoro-phenyl)-5-methyl-1H-pyrazol-4-yl|-thiazole-4-carboxylic acid (pyridin-3-ylmethyl)-amide

The title compound was prepared analogously to Example 72. ¹H NMR (400 MHz, DMSO-d₆) δ: 13.22 (bs, 1H), 8.83 (t, 1H), 8.61 (bs, 1H), 8.54 (m, 1H), 8.45 (m, 1H), 8.17 (m, 1H), 7.84 (m, 1H), 7.71 (m, H), 7.53 (m, 2H), 7.42 (m, 1H), 7.36 (m, 1H), 7.24 (m, 2H), 4.49 (d, 2H), 3.98 (d, 1H), 3.59 (bs, 1H), 3.37 (bs, 3H), 2.51 (s, 1H), 1.76 (bs, 1H). LCMS: 394.42 (M+1)⁺.

### EXAMPLE 77

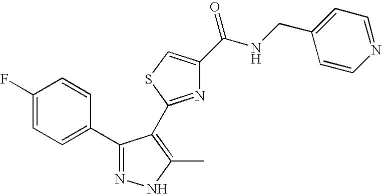

### 2-|3-(4-Fluoro-phenyl)-5-methyl-1H-pyrazol-4-yl|-thiazole-4-carboxylic acid (pyridin-4-ylmethyl)-amide

The title compound was prepared analogously to Example 72. ¹H NMR (400 MHz, DMSO-d₆) δ: 13.20 (s, 1H), 8.84 (bs, 1H), 8.50 (d, 2H), 8.18 (s, 1H), 7.54 (bs, 2H), 7.29 (m, 4H), 4.51 (d, 2H), 3.33 (bs, 1H), 2.47 (s, 1H), 1.10 (bs, 1H). LCMS: 394.7 (M+1)⁺.

### EXAMPLE 78

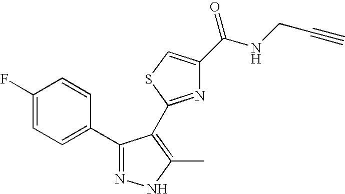

### 2-|3-(4-Fluoro-phenyl)-5-methyl-1H-pyrazol-4-yl|-thiazole-4-carboxylic acid prop-2-ynylamide

The title compound was prepared analogously to Example 72. ¹H NMR (400 MHz, DMSO-d₆) δ: 13.20 (s, 1H), 8.56 (bs, 1H), 8.16 (s, 1H), 7.51 (bs, 2H), 7.32 (m, 2H), 4.04 (d, 2H), 3.33 (s, 3H), 3.11 (s, 1H), 2.45 (bs, 1H), 1.78 (bs, 1H), 1.14 (bs, 1H). LCMS: 341.40 (M+1)⁺.

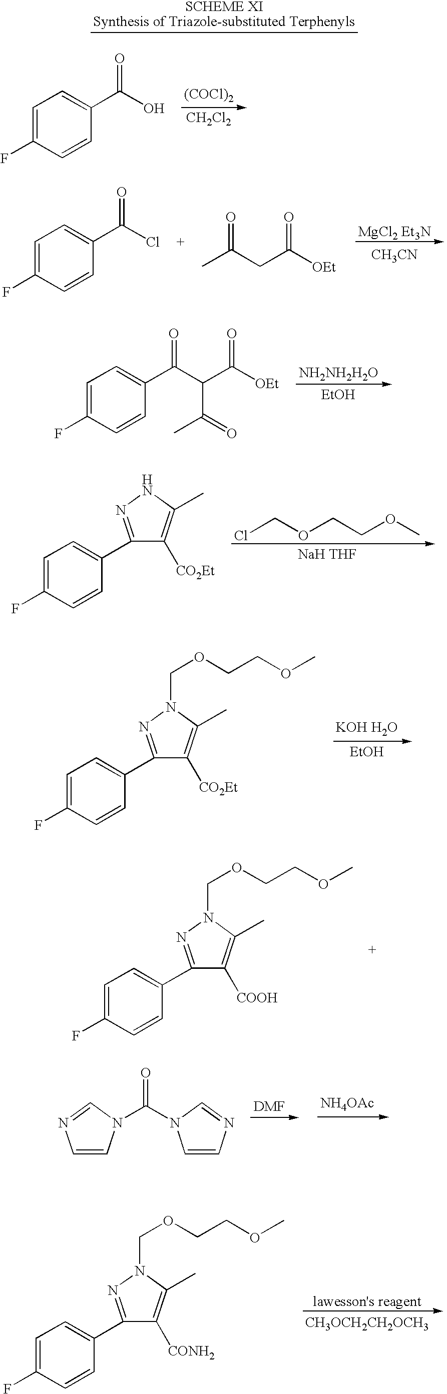

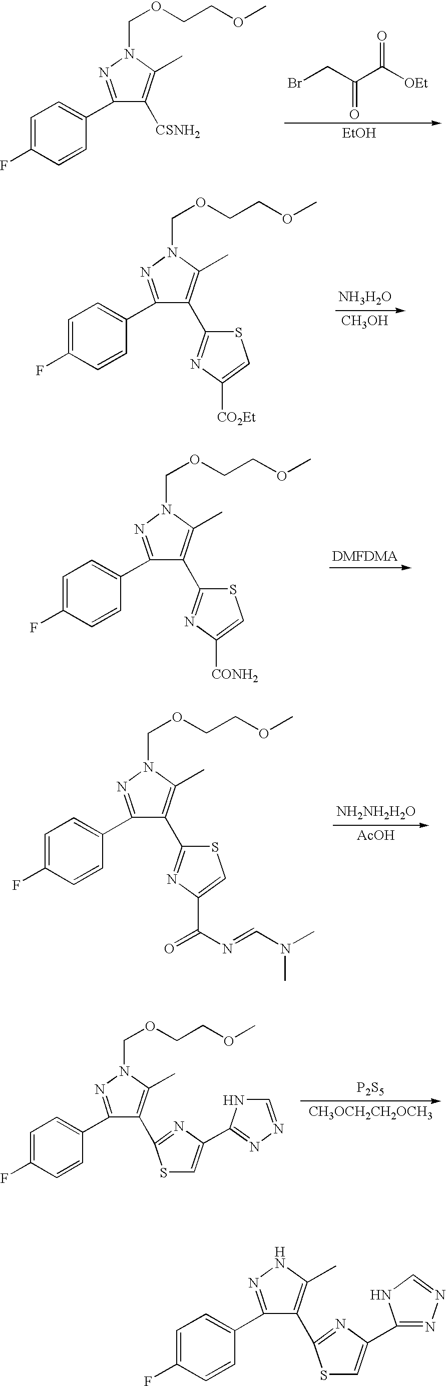

Example 79 was intentionally skipped

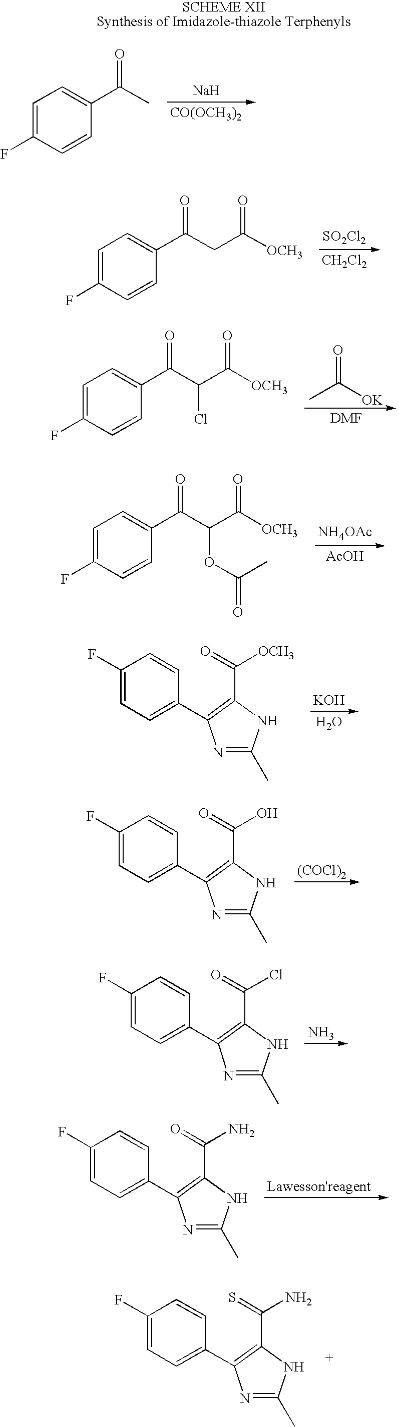

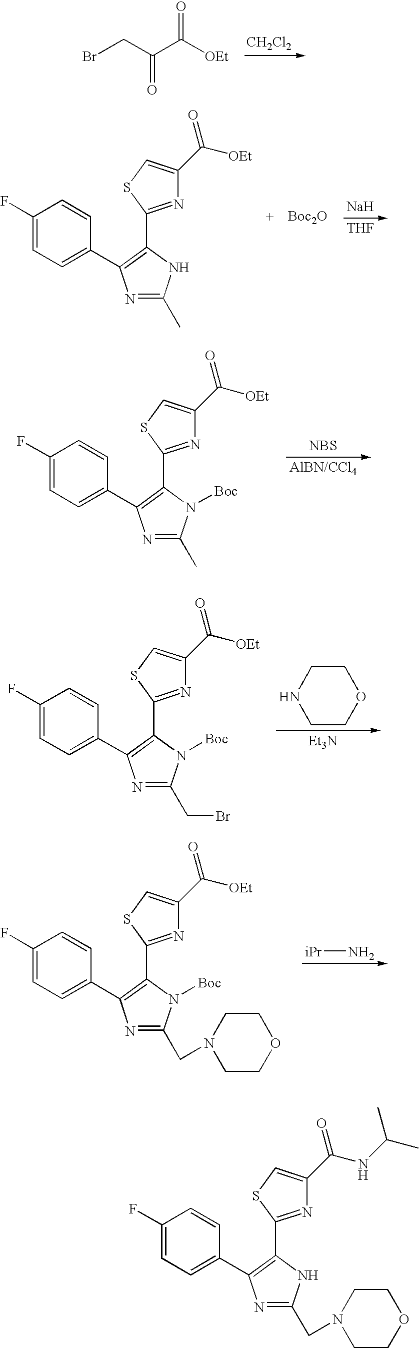

### EXAMPLE 80

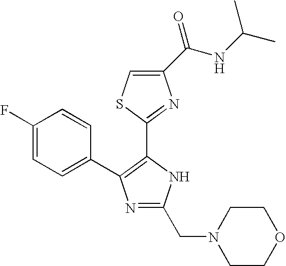

### 2-|5-(4-Fluoro-phenyl)-2-morpholin-4-ylmethyl-3H-imidazol-4-yl|-thiazole-4-carboxylic acid isopropylamide

Step 1

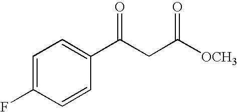

### 3-(4-Fluoro-phenyl)-3-oxo-propionic acid methyl ester

A 500 mL 3-necked round bottom flask, was charged with a solution of dimethyl carbonate (88.7 g, 975.70 mmol) and THF (300 mL). To the solution was added sodium hydride (37 g, 925.00 mmol). To the above was added 1-(4-fluorophenyl)ethanone (80 g, 573.91 mmol) dropwise with stirring, while warming to 60° C. over 30 minutes. The resulting solution was allowed to react for 2 hours while the temperature was maintained at 60° C. Work-up: pH was adjusted to −6 by the addition of HCl (18%). The resulting solution was extracted four times with 200 mL of diethylether and the organic layers combined. The resulting mixture was washed 2 times with 100 mL of brine, dried over Na₂SO₄ and concentrated by rotary evaporator. The final product was purified by distillation under reduced pressure (0.05 mm Hg). Product was collected at 100° C., resulting in 85 g (73%) of product as a light-yellow oil.
Step 2

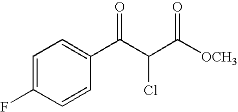

### 2-Chloro-3-(4-fluoro-phenyl)-3-oxo-propionic acid methyl ester

A 500 mL round bottom flask, was charged with 3-(4-Fluoro-phenyl)-3-oxo-propionic acid methyl ester (66 g, 323.27 mmol), and CCl₄ (200 mL). To the above was added sulfuryl dichloride (45.0 g, 330.00 mmol) dropwise with stirring, while maintaining the reaction at room temperature over a time period of 30 minutes. The resulting solution was allowed to react, for 4 hours at room temperature. The mixture was concentrated via rotary evaporator, resulting in 75 g (96%) of product as a light yellow oil.
Step 3

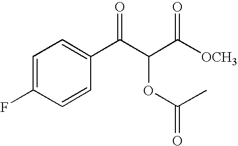

### 2-Acetoxy-3-(4-fluoro-phenyl)-3-oxo-propionic acid methyl ester

A 250 mL round bottom flask was charged with 2-Chloro-3-(4-fluoro-phenyl)-3-oxo-propionic acid methyl ester (25 g, 97.83 mmol), DMF (80 mL), and potassium acetate (21.3 g, 215.17 mmol). The resulting mixture was stirred overnight at room temperature. The reaction progress was monitored by TLC (EtOAc/Petroleum ether=1:4). Work-up: the mixture was poured into ice water (100 mL), and extracted three times with 300 mL of EtOAc. The resulting organics were washed 2 times with 100 mL of brine, dried over Na₂SO₄ and concentrated via rotary evaporator, resulting in 25 g (80%) of product as an orange oil.
Step 4

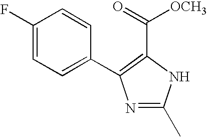

### 5-(4-Fluoro-phenyl)-2-methyl-3H-imidazole-4-carboxylic acid methyl ester

A 250 mL round bottom flask, was charged with methyl 2-Acetoxy-3-(4-fluoro-phenyl)-3-oxo-propionic acid methyl ester (5 g, 19.67 mmol), ammonium acetate (15.2 g, 196.7 mmol), and Acetic acid (30 mL). The resulting solution was allowed to reflux overnight, monitoring by TLC (EtOAc/Petroleum ether=1:2). Work-up: the mixture was concentrated, dissolved in 100 mL of EtOAc, washed 3 times with 30 mL of NaHCO3 (10%), dried over Na2SO4, and purified via column chromatography with a 1:10 EtOAc/Petroleum ether. This resulted in 2.8 g (60%) of product as a yellow solid.
Step 5

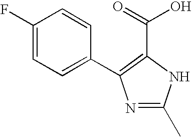

### 5-(4-Fluoro-phenyl)-2-methyl-3H-imidazole-4-carboxylic acid

A 250 mL round bottom flask was charged with 5-(4-Fluoro-phenyl)-2-methyl-3H-imidazole-4-carboxylic acid methyl ester (10 g, 42.74 mmol) and EtOH (100 mL). To the solution was added a solution of KOH (7.2 g, 128.57 mmol) in H₂O (40 mL). The resulting solution was refluxed overnight. The reaction progress was monitored by TLC (EtOAc/Petroleum ether=1:1). Work-up: the reaction pH was adjusted to 6-7 with 1N HCl. The resulting mixture was concentrated, dissolved in 200 mL of EtOH, and filtered. The filtrate was concentrated resulting in 7 g (74%) of carboxylic acid product as a white solid.
Step 6

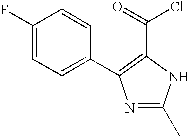

### 5-(4-Fluoro-phenyl)-2-methyl-3H-imidazole-4-carbonyl chloride

A 250 mL round bottom flask charged with 5-(4-Fluoro-phenyl)-2-methyl-3H-imidazole-4-carboxylic acid (4.8 g, 21.82 mmol), DCM (150 mL), (COCl)₂ (27.7 g, 218.23 mmol), and DMF (1 mL). The resulting solution was stirred overnight at room temperature. Reaction progress was monitored by TLC (DCM/MeOH=10:1). Work-up: the mixture was concentrated and taken-on to the next step without further purification.
Step 7

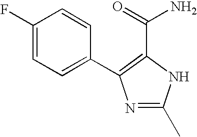

### 5-(4-Fluoro-phenyl)-2-methyl-3H-imidazole-4-carboxylic acid amide

A 250 mL round bottom flask was charged with 5-(4-Fluoro-phenyl)-2-methyl-3H-imidazole-4-carbonyl chloride (5.2 g, 21.85 mmol), DCM (150 mL), and NH₃ (gas, 18.6 g). The resulting solution was stirred for 2 hours at room temperature. The reaction progress was monitored by TLC (DCM/MeOH=10:1). Work-up: the mixture was concentrated to a solid that was washed with 20 mL of H₂O, and dried in an oven under reduced pressure, resulting in 2.6 g (54%) of product as a yellow solid.
Step 8

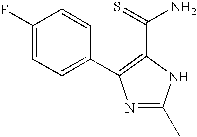

### 5-(4-Fluoro-phenyl)-2-methyl-3H-imidazole-4-carbothioic acid amide

A 250 mL round bottom flask was charged with 4-(4-fluorophenyl)-2-methyl-1H-imidazole-5-carboxamide (4.60 g, 21.00 mmol), DME (100 mL) and Lawesson's reagent (12.74 g, 31.53 mmol). The resulting solution stirred overnight in a 60° C. oil bath. Reaction progress was monitored by TLC (DCM/MeOH=10:1). The resulting mixture was concentrated and purified by chromatography through a neutral aluminum oxide eluted with a 1:10 EtOAc/Petroleum ether solvent system. This resulted in 3.1 g (63%) of product as a yellow solid.
Step 9

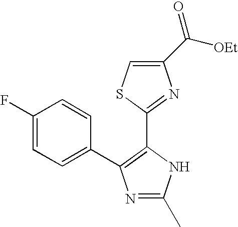

### 2-|5-(4-Fluoro-phenyl)-2-methyl-3H-imidazol-4-yl|-thiazole-4-carboxylic acid ethyl ester

A 250 mL round bottom flask, was charged with 5-(4-Fluoro-phenyl)-2-methyl-3H-imidazole-4-carbothioic acid amide (4.6 g, 19.57 mmol), ethyl 3-bromo-2-oxopropanoate (5.73 g, 29.38 mmol), and EtOH (150 mL), then stirred at reflux for 3 h with heating from an oil bath. Reaction progress was monitored by TLC (EtOAc/Petroleum ether=1:1). Work-up: the mixture was concentrated and recrystallization from EtOAc, giving 4.7 g (72%) of product as a white solid.
Step 10

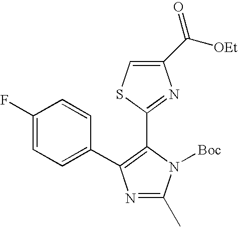

### 2-|3-tert-Butoxycarbonyl-5-(4-fluoro-phenyl)-2-methyl-3H-imidazol-4-yl|-thiazole-4-carboxylic acid ethyl ester

A 100 mL round bottom flask was charged with ethyl 2-[5-(4-Fluoro-phenyl)-2-methyl-3H-imidazol-4-yl]-thiazole-4-carboxylic acid ethyl ester (200 mg, 0.60 mmol), THF (30 mL), and NaH (40 mg, 1.67 mmol), then stirred at room temperature for 1 h. This mixture treated with di-tert-butyl dicarbonate (158 mg, 0.72 mmol), and stirred for an additional 2 h at room temperature. Reaction progress was monitored by TLC (EtOAc/Petroleum ether=1:1; Rf=0.4). Work-up: the mixture was concentrated, dissolved in 100 mL of diethyl ether, washed 2 times with 30 mL of water, dried over Na₂SO₄, filtered, and concentrated, giving 0.3 g of crude product a as yellow oil.
Step 11

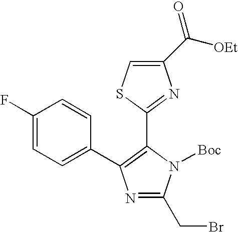

### 2-|2-Bromomethyl-3-tert-butoxycarbonyl-5-(4-fluoro-phenyl)-3H-imidazol-4-yl|-thiazole-4-carboxylic acid ethyl ester

A 100 mL round bottom flask was charged with 2-[3-tert-Butoxycarbonyl-5-(4-fluoro-phenyl)-2-methyl-3H-imidazol-4-yl]-thiazole-4-carboxylic acid ethyl ester (300 mg, 0.70 mmol), NBS (120 mg, 0.67 mmol), AIBN (20 mg, 0.12 mmol), and CCl₄ (40 mL). The resulting solution was stirred for 3 hours at reflux, while monitoring by TLC (EtOAc/Petroleum ether=1:3, Rf=0.3). The residue was dissolved in 50 mL of DCM, washed 3 times with 30 mL of water, dried over Na₂SO₄, filtered, and concentrated, giving in 0.3 g (crude) of product as a yellow oil. This material was used in the next step without further purification.
Step 12

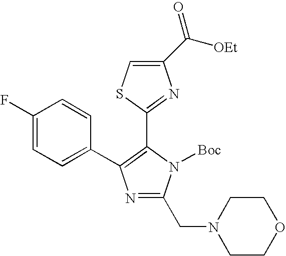

### 2-|3-tert-Butoxycarbonyl-5-(4-fluoro-phenyl)-2-morpholin-4-ylmethyl-3H-imidazol-4-yl|-thiazole-4-carboxylic acid ethyl ester

A 100 mL round bottom flask was charged with 2-[2-Bromomethyl-3-tert-butoxycarbonyl-5-(4-fluoro-phenyl)-3H-imidazol-4-yl]-thiazole-4-carboxylic acid ethyl ester (300 mg, 0.59 mmol), triethylamine (300 mg, 2.96 mmol), morpholine (160 mg, 1.84 mmol), and EtOH (50 mL). The resulting solution was stirred for 2 hours at room temperature, and monitored by TLC (EtOAc/Petroleum ether=1:2, Rf=0.1). The mixture was then dissolved in 100 mL of AcOEt, and washed 3 times with 20 mL of water. The resulting solution was dried over Na₂SO₄, filtered, concentrated, and purified by column chromatography eluting with a 1:5 EtOAc/Petroleum ether, giving 100 mg (33%) of product as a yellow solid.
Step 13

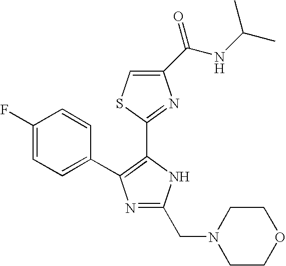

### 2-|5-(4-Fluoro-phenyl)-2-morpholin-4-ylmethyl-3H-imidazol-4-yl|-thiazole-4-carboxylic acid isopropylamide

A 10 mL sealed tube was charged with 2-[3-tert-Butoxycarbonyl-5-(4-fluoro-phenyl)-2-morpholin-4-ylmethyl-3H-imidazol-4-yl]-thiazole-4-carboxylic acid ethyl ester (100 mg, 0.19 mmol), and propan-2-amine (5 mL), then stirred for 48 hours at 65° C. with heating from an oil bath. Reaction progress was monitored by TLC (DCM/MeOH=10:1, Rf=0.3). Work-up: the mixture was concentrated, and purified by column chromatography, eluting with a 1:5 EtOAc/Petroleum ether, giving 60 mg (72%) of product as a white solid. ¹H NMR (400 MHz, CDCl₃) δ: 7.92 (s, 1H), 7.70 (m, 2H), 7.19 (m, 2H), 4.22 (m, 1H), 2.55 (s, 4H), 1.50-1.70 (m, 6H), 1.22 (d, 6H).

### EXAMPLE 81

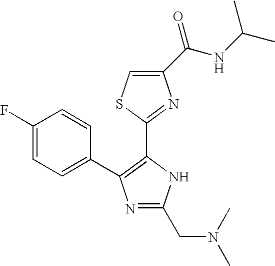

### 2-|2-Dimethylaminomethyl-5-(4-fluoro-phenyl)-3H-imidazol-4-yl|-thiazole-4-carboxylic acid isopropylamide

The title compound was prepared analogously to 2-[5-(4-Fluoro-phenyl)-2-morpholin-4-ylmethyl-3H-imidazol-4-yl]-thiazole-4-carboxylic acid isopropylamide, where dimethylamine hydrochloride was substituted for morpholine in step 12 of that sequence. ¹H NMR (400 MHz, CDCl₃) δ 9.50-10.50 (bs, 1H), 7.85 (m, 2H), 7.14 (t, 2H), 7.19 (m, 2H), 6.95 (s, 1H), 4.19 (s, 1H), 3.75 (s, 2H), 2.38 (s, 6H), 1.23 (s, 6H).

### EXAMPLE 82

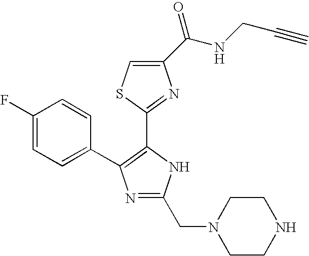

### 2-|5-(4-Fluoro-phenyl)-2-piperazin-1-ylmethyl-3H-imidazol-4-yl|-thiazole-4-carboxylic acid prop-2-ynylamide

Step 1

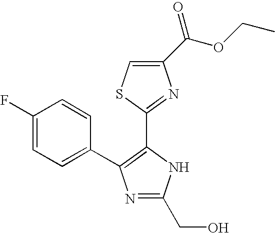

### 2-|5-(4-Fluoro-phenyl)-2-hydroxymethyl-3H-imidazol-4-yl|-thiazole-4-carboxylic acid ethyl ester

A 50 mL round bottom flask was charged with tert-butyl 2-[2-Bromomethyl-3-tert-butoxycarbonyl-5-(4-fluoro-phenyl)-3H-imidazol-4-yl]-thiazole-4-carboxylic acid ethyl ester (150 mg, 0.29 mmol, prepared as described in Step 11 of Example 80), DMSO (20 mL), and H₂O (5 mL). The resulting solution stirred for 3 hours at 80° C. Reaction progress was monitored by TLC (DCM/MeOH=10:1). Work-up: the reaction was concentrated, dissolved in 100 mL of EtOAc, washed 3 times with 20 mL of H₂O, dried over Na₂SO₄ and concentrated. This gave 100 mg (98%) of ethyl 2-(4-(4-fluorophenyl)-2-(hydroxymethyl)-1H-imidazol-5-yl)thiazole-4-carboxylate as a white solid.
Step 2

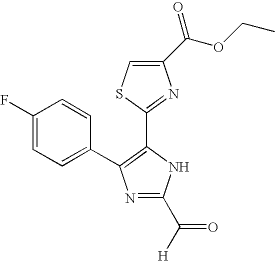

### 2-|5-(4-Fluoro-phenyl)-2-formyl-3H-imidazol-4-yl|-thiazole-4-carboxylic acid ethyl ester

A 25 mL round bottom flask was charged with 2-[5-(4-Fluoro-phenyl)-2-hydroxymethyl-3H-imidazol-4-yl]-thiazole-4-carboxylic acid ethyl ester (160 mg, 0.461 mmol), Dess-Martin periodinane (235 mg, 0.553 mmol), and DCM (3 mL). The resulting solution was stirred at room temperature for 1 hr. Work-up: mixture was filtered to remove insoluble IBX, concentrated, and used in the next step without further purification. (M+1⁺): 345.73
Step 3

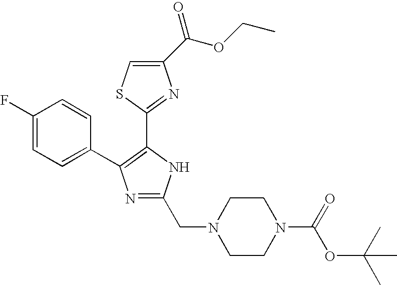

### 4-|5-(4-Ethoxycarbonyl-thiazol-2-yl)-4-(4-fluoro-phenyl)-1H-imidazol-2-ylmethyl|-piperazine-1-carboxylic acid tert-butyl ester

A 25 mL round bottom flask was charged with crude 2-[5-(4-Fluoro-phenyl)-2-formyl-3H-imidazol-4-yl]-thiazole-4-carboxylic acid ethyl ester (0.461 mmol), piperazine-1-carboxylic acid tert-butyl ester (103 mg, 0.553 mmol), sodium triacetoxy borohydride (136 mg, 0.645 mmol), and DCM (3.0 mL). The mixture was stirred at room temperature for 1 hr, at which time LCMS indicated complete conversion. Work-up: the reaction was filtered through celite, and concentrated to an oil (442 mg, theoretical=237 mg), and used in the next step without further purification. LCMS (M+1⁺): 516.01
Step 4

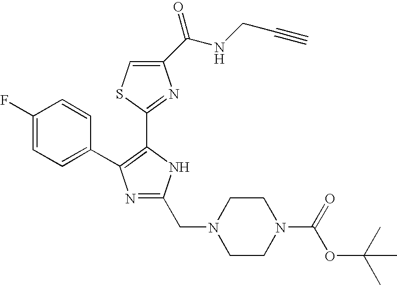

### 4-|4-(4-Fluoro-phenyl)-5-(4-prop-2-ynylcarbamoyl-thiazol-2-yl)-1H-imidazol-2-ylmethyl|-piperazine-1-carboxylic acid tert-butyl ester

A 25 mL round bottom flask was charged with crude 4-[5-(4-Ethoxycarbonyl-thiazol-2-yl)-4-(4-fluoro-phenyl)-1H-imidazol-2-ylmethyl]-piperazine-1-carboxylic acid tert-butyl ester (0.46 mmol), MeAlCl(NH-propargyl) (1.37 mL of 0.67 M solution prepared as described in Synthetic Communications, 12 (13), 989-993 (1982)), and toluene (1.4 mL). The resulting solution was heated in a 80° C. in a heating block until all starting material was consumed (1 hr), as indicated by LCMS. Work-up: the reaction was diluted with DCM (20 mL), and stirred with Na₂SO₄-10H₂O (5 g) for 1 hr, then filtered, concentrated to an oil, and used in the next step without further purification. LCMS (M+1⁺): 525.04
Step 5

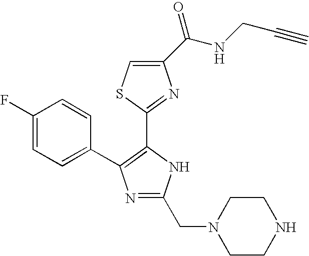

### 2-|5-(4-Fluoro-phenyl)-2-piperazin-1-ylmethyl-3H-imidazol-4-yl|-thiazole-4-carboxylic acid prop-2-ynylamide

A 25 mL round bottom flask was charged with crude 4-[4-(4-Fluoro-phenyl)-5-(4-prop-2-ynylcarbamoyl-thiazol-2-yl)-1H-imidazol-2-ylmethyl]-piperazine-1-carboxylic acid tert-butyl ester (0.46 mmol), and 1:1 TFA/DCM (10 mL). The resulting solution was stirred at room temperature for 20 min. diluted with toluene (10 mL), concentrated to an oil. The crude product was purified by C18 reverse phase semi-preparative HPLC, giving the product as white solid (mono TFA salt, 48.1 mg, 24% for four steps). ¹H NMR (400 MHz, DMSO-d₆) δ: 8.68 (bs, 2H), 8.23 (s, 1H), 8.05 (m, 3H), 7.33 (t, 2H), 4.10 (m, 2H), 4.60-5.40 (bm, 4H), 3.82 (s, 2H), 3.20 (s, 1H), 2.84 (bs, 4H). LCMS (M+1⁺): 426.28.

### EXAMPLE 83

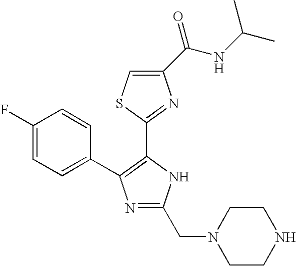

### 2-|5-(4-Fluoro-phenyl)-2-piperazin-1-ylmethyl-3H-imidazol-4-yl|-thiazole-4-carboxylic acid isopropylamide

The title compound was prepared analogously to 2-[5-(4-Fluoro-phenyl)-2-piperazin-1-ylmethyl-3H-imidazol-4-yl]-thiazole-4-carboxylic acid prop-2-ynylamide where MeAlCl(NH-isopropyl), was substituted for MeAlCl(NH-propargyl) in step 4 of that sequence. ¹H NMR (400 MHz, DMSO-d₆) δ: 8.65 (bs, 2H), 8.10 (s, 1H), 7.92 (m, 2H), 3.96 (m, 1H), 3.75 (s, 2H), 3.13 (m, 4H), 2.74 (m, 4H), 1.06 (d, 6H). LCMS (M+1⁺): 428.83

### EXAMPLE 84

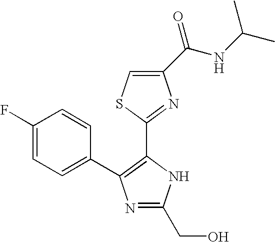

### 2-|5-(4-Fluoro-phenyl)-2-hydroxymethyl-3H-imidazol-4-yl|-thiazole-4-carboxylic acid isopropylamide

The title compound was prepared by amidation (iPrNH₂, sealed tube) of 2-[5-(4-Fluoro-phenyl)-2-hydroxymethyl-3H-imidazol-4-yl]-thiazole-4-carboxylic acid ethyl ester (described in step 1 of Example 82).

### EXAMPLE 85

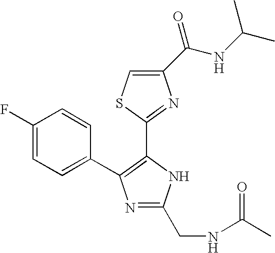

### 2-|2-(Acetylamino-methyl)-5-(4-fluoro-phenyl)-3H-imidazol-4-yl|-thiazole-4-carboxylic acid isopropylamide

The title compound was prepared from 2-[2-Bromomethyl-3-tert-butoxycarbonyl-5-(4-fluoro-phenyl)-3H-imidazol-4-yl]-thiazole-4-carboxylic acid ethyl ester (described in step 11 of Example 80. The bromide was displaced with sodium azide, reduced with catalytic hydrogenation, and acylated with actetic anhydride.

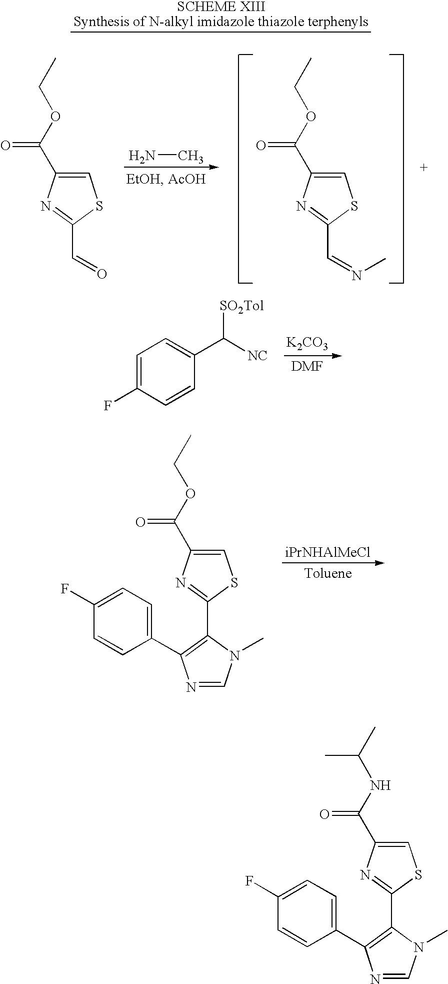

### EXAMPLE 86

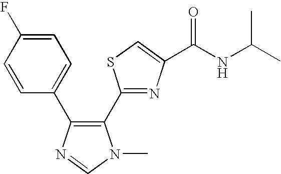

### 2-|5-(4-Fluoro-phenyl)-3-methyl-3H-imidazol-4-yl|-thiazole-4-carboxylic acid isopropylamide

Step 1:

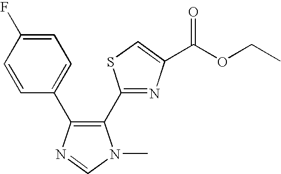

### 2-|5-(4-Fluoro-phenyl)-3-methyl-3H-imidazol-4-yl|-thiazole-4-carboxylic acid ethyl ester

To a stirred solution of 2-formyl-thiazole-4-carboxylic acid ethyl ester (100 mg, 0.54 mmol, prepared analogously to the literature method described in J. Org. Chem. 1997, 62, 3804, but using Swern oxidation methodology to form the aldehyde instead of PCC), and AcOH (10 μL) in EtOH (4 mL) at room temperature was added methylamine (2.0 mmol, 1 mL of a 2 M solution in THF). The reaction mixture was warmed to 60° C. and left to stir for 10 min, after which time TLC analysis (30% EtOAc in hexanes, triethylamine-pretreated plate) revealed disappearance of starting aldehyde. The reaction was cooled to room temperature and evaporated to dryness in vacuo. The crude mixture of isomeric imine products was then redissolved in DMF (4 mL), treated with [(4-fluoro-phenyl)-(toluene-4-sulfonyl)-methyl]-isocyanide (218 mg, 0.75 mmol, prepared analogously to the literature method described in J. Org. Chem. 1998, 63, 4529) and K₂CO₃ (372 mg, 2.7 mmol) and left to stir at room temperature for 18 hrs, after which time LC/MS analysis of the mixture revealed significant conversion of reactants to the title compound. The resulting crude reaction was poured in to 1:1 EtOAc:hexanes (100 mL), extracted with 5% NaCl (4×50 mL), dried over MgSO₄, filtered, and concentrated in vacuo. The crude residue was then purified by SiO₂ flash chromatography, eluting with EtOAc to afford the title compound (178 mg, 100% yield) as an off white solid. ¹H NMR (400 MHz, CDCl₃) δ 8.13 (s, 1H), 7.60 (s, 1H), 7.47 (m, 2H), 7.05 (m, 2H), 4.44 (q, 2H), 3.89 (s, 3H), 1.43 (t, 3H). LCMS: 331.7 (M+1)⁺.
Step 2

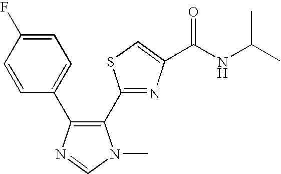

### 2-|5-(4-Fluoro-phenyl)-3-methyl-3H-imidazol-4-yl|-thiazole-4-carboxylic acid isopropylamide

To a stirred solution of 2-[5-(4-fluoro-phenyl)-3-methyl-3H-imidazol-4-yl]-thiazole-4-carboxylic acid ethyl ester (50 mg, 0.15 mmol) in toluene (2 mL), was added MeAlCl(NH-iPr) (450 μL of a 0.67 M solution in toluene, 0.30 mmol, prepared as described in Synth. Comm. 12, 13, 989.) dropwise via syringe. The resulting mixture was warmed to 80° C. and left to stir for 2 hrs, then cooled to room temperature and poured on to a vigorously stirred slurry of sodium sulfate decahydrate (5 g) in DCM (40 mL). After 1 hr. the mixture was filtered, and the resulting filtrate was dried over MgSO₄, filtered, and concentrated in vacuo to afford the title compound (45 mg, 87% yield), as a tan solid that was determined to be sufficiently pure by available analytical methods. ¹H NMR (400 MHz, CDCl₃) δ 8.09 (s, 1H), 7.62 (s, 1H), 7.48 (m, 2H), 7.05 (m, 3H), 4.28 (m, 1H), 3.85 (s, 3H), 1.28 (d, 6H). LCMS: 345.2 (M+1)⁺.

### EXAMPLE 87

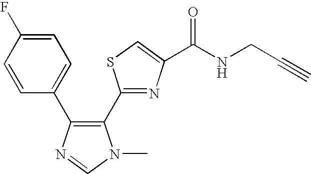

### 2-|5-(4-Fluoro-phenyl)-3-methyl-3H-imidazol-4-yl|-thiazole-4-carboxylic acid prop-2-ynylamide

The title compound was prepared analogously to 2-[5-(4-fluoro-phenyl)-3-methyl-3H-imidazol-4-yl]-thiazole-4-carboxylic acid isopropylamide, where MeAlCl(NH-Propargyl) was substituted for MeAlCl(NH-iPr) in step 2 of that sequence. ¹H NMR (400 MHz, CD₃OD) δ 8.86 (s, 1H), 8.34 (s, 1H), 7.56 (m, 2H), 7.26 (m, 2H), 4.20 (d, 2H), 4.14 (s, 3H). LCMS: 341.0 (M+1)⁺.

### EXAMPLE 88

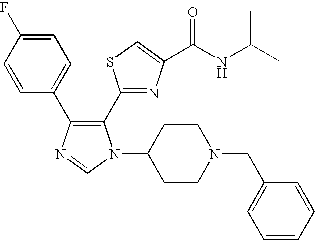

### 2-|3-(1-Benzyl-piperidin-4-yl)-5-(4-fluoro-phenyl)-3H-imidazol-4-yl|-thiazole-4-carboxylic acid isopropylamide

The title compound was prepared analogously to 2-[5-(4-fluoro-phenyl)-3-methyl-3H-imidazol-4-yl]-thiazole-4-carboxylic acid isopropylamide, where 4-Amino-1-benzylpiperidine was substituted for methylamine in step 1 of that sequence. ¹H NMR (400 MHz, CDCl₃) δ 8.10 (s, 1H), 7.81 (s, 1H), 7.45 (m, 2H), 7.26-7.37 (m, 5H), 7.03 (m, 3H), 4.40 (m, 1H), 4.24 (m, 1H), 3.58 (s, 2H), 3.09 (m, 2H), 2.0-2.22 (m, 6H), 1.23 (d, 6H). LCMS: 504.6 (M+1)⁺.

### EXAMPLE 89

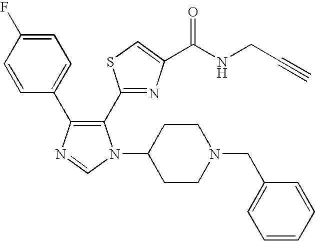

### 2-|3-(1-Benzyl-piperidin-4-yl)-5-(4-fluoro-phenyl)-3H-imidazol-4-yl|-thiazole-4-carboxylic acid prop-2-ynylamide

The title compound was prepared analogously to 2-[5-(4-fluoro-phenyl)-3-methyl-3H-imidazol-4-yl]-thiazole-4-carboxylic acid isopropylamide, where 4-Amino-1-benzylpiperidine was substituted for methylamine in step 1 of that sequence, and (propargyl-NH)AlMeCl was substituted for MeAlCl(NH-iPr) in step 2 of that sequence. ¹H NMR (400 MHz, CD₃OD) δ 8.29 (s, 1H), 8.10 (s, 1H), 7.43 (m, 2H), 7.25-7.35 (m, 5H), 7.10 (m, 3H), 4.60 (m, 1H), 4.21 (d, 2H), 3.58 (s, 2H), 3.05 (m, 2H), 2.67 (m, 1H), 2.0-2.22 (m, 6H). LCMS: 500.6 (M+1)⁺.

### EXAMPLE 90

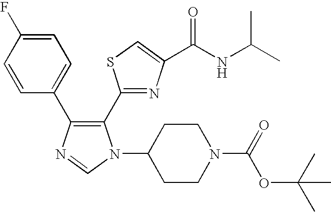

### 4-|4-(4-Fluoro-phenyl)-5-(4-isopropylcarbamoyl-thiazol-2-yl)-imidazol-1-yl|-piperidine-1-carboxylic acid tert-butyl ester

The title compound was prepared analogously to 2-[5-(4-Fluoro-phenyl)-3-methyl-3H-imidazol-4-yl]-thiazole-4-carboxylic acid isopropylamide, where tert-butyl 4-amino-1-piperidinecarboxylate was substituted for methylamine in step 1 of that sequence. ¹H NMR (400 MHz, CDCl₃) δ 8.13 (s, 1H), 7.76 (s, 1H), 7.44 (m, 2H), 7.04 (m, 3H), 4.48 (m, 1H), 4.25-4.38 (m, 3H), 2.75 (m, 2H), 2.18 (m, 2H), 1.90 (m, 2H), 1.48 (s, 9H), 1.28 (d, 6H). LCMS: 514.6 (M+1)⁺.

### EXAMPLE 91

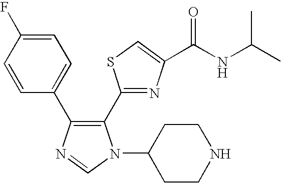

### 2-|5-(4-Fluoro-phenyl)-3-piperidin-4-yl-3H-imidazol-4-yl|-thiazole-4-carboxylic acid isopropylamide

To a stirred solution of 4-[4-(4-fluoro-phenyl)-5-(4-isopropylcarbamoyl-thiazol-2-yl)-imidazol-1-yl]-piperidine-1-carboxylic acid tert-butyl ester (51 mg, 0.10 mmol) in DCM (10 mL), at room temperature, was added trifluoroacetic acid (2 mL). After 30 minutes, TLC analysis revealed the disappearance of the BOC starting material. The reaction was diluted with DCM (100 mL) and toluene (50 mL), and then concentrated to dryness in vacuo. The crude residue was purified by automated C18 reverse phase semi-preparative HPLC to afford the title compound (47 mg, mono-TFA salt) as a pale yellow semi-solid. ¹H NMR (400 MHz, CD₃OD) δ 9.22 (s, 1H), 8.36 (s, 1H), 7.54 (m, 2H), 7.26 (m, 2H), 5.18 (m, 1H), 4.22 (m, 1H), 3.62 (d, 2H), 3.21 (m, 2H), 2.58 (d, 2H), 2.35 (m, 2H), 1.29 (d, 6H). LCMS: 414.6 (M+1)⁺.

### EXAMPLE 92

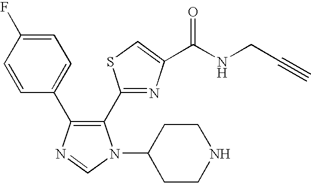

### 2-|5-(4-Fluoro-phenyl)-3-piperidin-4-yl-3H-imidazol-4-yl|-thiazole-4-carboxylic acid prop-2-ynylamide

Step 1

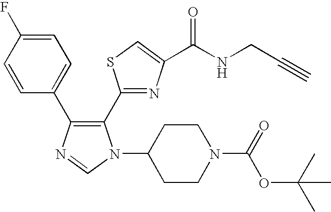

### 4-|4-(4-Fluoro-phenyl)-5-(4-prop-2-ynylcarbamoyl-thiazol-2-yl)-imidazol-1-yl|-piperidine-1-carboxylic acid tert-butyl ester

The title compound was prepared analogously to 2-[5-(4-fluoro-phenyl)-3-methyl-3H-imidazol-4-yl]-thiazole-4-carboxylic acid isopropylamide, where tert-butyl 4-amino-1-piperidinecarboxylate was substituted for methylamine in step 1 of that sequence, and (propargyl-NH)AlMeCl was substituted for MeAlCl(NH-iPr) in step 2 of that sequence. LCMS: 510.8 (M+1)⁺.
Step 2

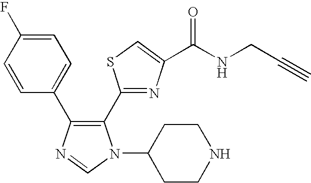

### 2-|5-(4-Fluoro-phenyl)-3-piperidin-4-yl-3H-imidazol-4-yl|-thiazole-4-carboxylic acid prop-2-ynylamide

To a stirred solution of 4-[4-(4-fluoro-phenyl)-5-(4-prop-2-ynylcarbamoyl-thiazol-2-yl)-imidazol-1-yl]-piperidine-1-carboxylic acid tert-butyl ester (56 mg, 0.11 mmol) in DCM (10 mL) at room temperature was added trifluoroacetic acid (2 mL). After 1 hr, TLC analysis revealed the disappearance of the BOC starting material. The reaction was diluted with DCM (100 mL) and toluene (50 mL) and concentrated to dryness in vacuo. The crude residue was purified by automated C18 reverse phase semi-preparative HPLC to afford the title compound (50 mg, mono-TFA salt) as a tan semi-solid. ¹H NMR (400 MHz, CD₃OD) δ 8.53 (s, 1H), 8.33 (s, 1H), 7.47 (m, 2H), 7.21 (m, 2H), 5.07 (m, 1H), 4.21 (d, 2H), 3.59 (d, 2H), 3.21 (m, 2H), 2.65 (m, 1H), 2.52 (d, 2H), 2.25 (m, 2H). LCMS: 410.6 (M+1)⁺.

### EXAMPLE 93

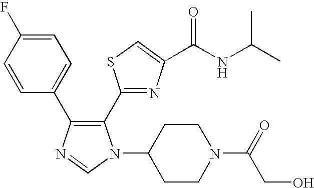

### 2-{5-(4-Fluoro-phenyl)-3-|1-(2-hydroxy-acetyl)-piperidin-4-yl|-3H-imidazol-4-yl}-thiazole-4-carboxylic acid isopropylamide

To a stirred solution of 2-[5-(4-fluoro-phenyl)-3-piperidin-4-yl-3H-imidazol-4-yl]-thiazole-4-carboxylic acid isopropylamide (43 mg, 0.10 mmol) and glycolic acid (33 mg, 0.45 mmol) in DCM (2 mL), at room temperature, was added N-(3-dimethylaminopropyl)-N′-ethylcarbodiimide hydrochloride (EDC, 93 mg, 0.50 mmol). After 1 hour, LC/MS analysis revealed the disappearance of starting material. The crude mixture was concentrated in vacuo, redissolved in MeOH (3 mL) and LiOH (1 mL of a 1N aqueous solution), and stirred for an additional 30 min. The pH was adjusted to pH-6 via the addition of 1N HCl, and the resulting mixture was concentrated to dryness in vacuo and purified by automated C18 reverse phase semi-preparative HPLC to afford the title compound (38 mg, 81%) as an off white solid. LCMS: 472.1 (M+1)⁺.

### EXAMPLE 94

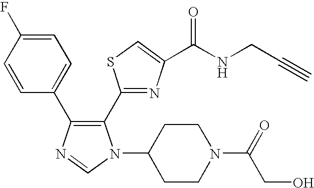

### 2-{5-(4-Fluoro-phenyl)-3-|1-(2-hydroxy-acetyl)-piperidin-4-yl|-3H-imidazol-4-yl}-thiazole-4-carboxylic acid prop-2-ynylamide

The title compound was prepared analogously to 2-{5-(4-fluoro-phenyl)-3-[1-(2-hydroxy-acetyl)-piperidin-4-yl]-3H-imidazol-4-yl}-thiazole-4-carboxylic acid isopropylamide, where 2-[5-(4-fluoro-phenyl)-3-piperidin-4-yl-3H-imidazol-4-yl]-thiazole-4-carboxylic acid prop-2-ynylamide was substituted for 2-[5-(4-fluoro-phenyl)-3-piperidin-4-yl-3H-imidazol-4-yl]-thiazole-4-carboxylic acid isopropylamide in that reaction. ¹H NMR (400 MHz, CD₃OD) δ 8.96 (s, 1H), 8.39 (s, 1H), 7.51 (m, 2H), 7.24 (m, 2H), 5.07 (m, 1H), 4.70 (d, 1H), 4.27 (d, 2H), 4.21 (d, 2H), 3.92 (d, 1H), 3.19 (m, 1H), 2.83 (m, 1H), 2.66 (m, 1H), 2.40 (m, 2H), 2.00 (m, 2H). LCMS: 468.3 (M+1)⁺.

### EXAMPLE 95

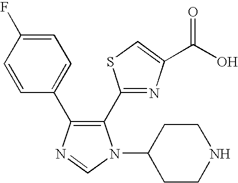

### 2-|5-(4-Fluoro-phenyl)-3-piperidin-4-yl-3H-imidazol-4-yl|-thiazole-4-carboxylic acid

Step 1

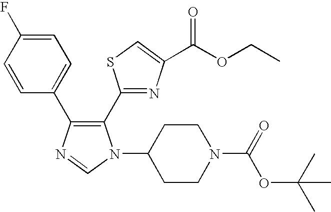

### 4-|5-(4-Ethoxycarbonyl-thiazol-2-yl)-4-(4-fluoro-phenyl)-imidazol-1-yl|-piperidine-1-carboxylic acid tert-butyl ester

The title compound was prepared analogously to 2-[5-(4-fluoro-phenyl)-3-methyl-3H-imidazol-4-yl]-thiazole-4-carboxylic acid ethyl ester, prepared as described in step 1 of Example 86, where tert-butyl 4-amino-1-piperidinecarboxylate was substituted for methylamine in that sequence. ¹H NMR (400 MHz, CDCl₃) δ 8.12 (s, 1H), 7.75 (s, 1H), 7.46 (m, 2H), 7.08 (m, 2H), 4.85 (m, 1H), 4.44 (q, 2H), 4.30 (m, 2H), 2.83 (m, 2H), 2.20 (m, 2H), 1.83 (m, 2H), 1.49 (s, 9H), 1.43 (t, 3H). LCMS: 501.3 (M+1)⁺.
Step 2

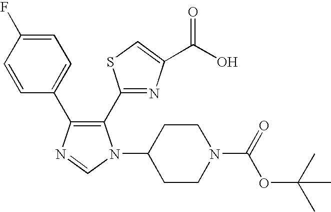

### 4-|5-(4-Carboxy-thiazol-2-yl)-4-(4-fluoro-phenyl)-imidazol-1-yl|-piperidine-1-carboxylic acid tert-butyl ester

To a stirred solution of 4-[5-(4-ethoxycarbonyl-thiazol-2-yl)-4-(4-fluoro-phenyl)-imidazol-1-yl]-piperidine-1-carboxylic acid tert-butyl ester (112 mg, 0.22 mmol) in MeOH (1 mL), at room temperature was added LiOH (330 μL of a 1N aqueous solution, 0.33 mmol). The reaction was stirred at room temperature and monitored by TLC for the disappearance of starting material. Upon completion, the reaction was neutralized with HCl (330 μL of a 1N aqueous solution, 0.33 mmol) and concentrated to dryness in vacuo to afford the title compound (100 mg, 97%) as a tan solid that was determined to be suitably pure by LCMS to carry on to the next step. LCMS: 473.2 (M+1)⁺.
Step 3

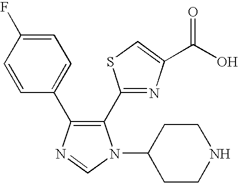

### 2-|5-(4-Fluoro-phenyl)-3-piperidin-4-yl-3H-imidazol-4-yl|-thiazole-4-carboxylic acid

To a stirred solution of 4-[5-(4-carboxy-thiazol-2-yl)-4-(4-fluoro-phenyl)-imidazol-1-yl]-piperidine-1-carboxylic acid tert-butyl ester (100 mg, 0.21 mmol) in DCM (1.0 mL) was added trifluoroacetic acid (2.0 mL of a 25% solution in DCM). After 45 min, LCMS analysis of the reaction mixture revealed disappearance of starting material, and significant conversion to the title compound. The reaction was diluted with DCM (100 mL) and toluene (50 mL) and concentrated to dryness in vacuo. The crude residue was then purified by automated C18 reverse phase semi-preparative HPLC to afford the title compound (78 mg, 76%, mono TFA salt) as a colorless solid. ¹H NMR (400 MHz, CD₃OD) δ 9.12 (s, 1H), 8.47 (s, 1H), 7.58 (m, 2H), 7.28 (m, 2H), 5.28 (m, 1H), 3.59 (d, 2H), 3.29 (m, 2H), 2.61 (d, 2H), 2.35 (m, 2H). LCMS: 373.4 (M+1)⁺.

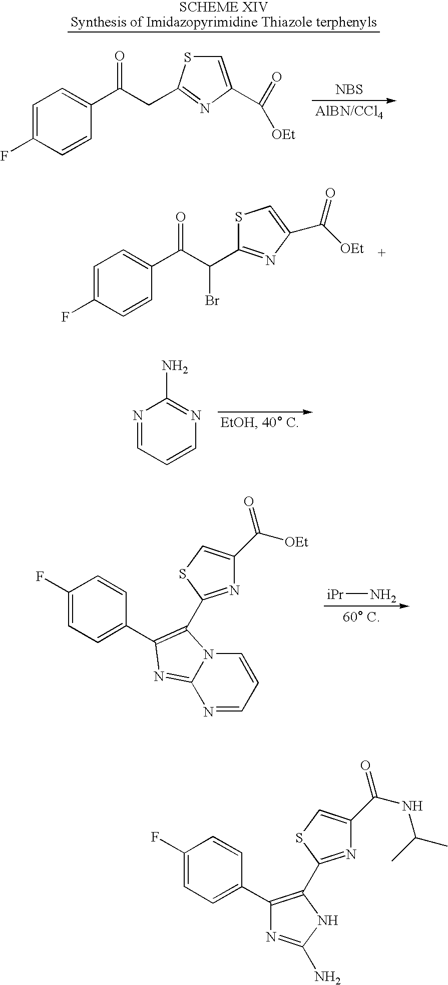

### EXAMPLE 96

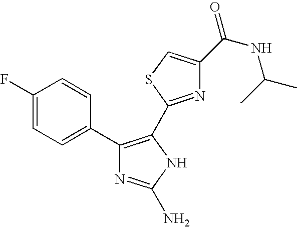

### 2-|2-Amino-5-(4-fluoro-phenyl)-3H-imidazol-4-yl|-thiazole-4-carboxylic acid isopropylamide

Step 1:

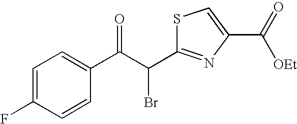

### 2-(4-Acetyl-thiazol-2-yl)-2-bromo-1-(4-fluoro-phenyl)-ethanone

A 500 ml round bottom flask was charged with ethyl 2-(2-(4-fluorophenyl)-2-oxoethyl)thiazole-4-carboxylate (14 g, 43.0 mmol) (prepared as described in step 3 of Example 61), CCl₄ (200 ml), NBS (7.65 g, 43.0 mmol), and AIBN (0.8 g, 4.87 mmol). The resulting solution was refluxed under light from a mercury vapor lamp for 2 hours. Reaction progress was monitored by TLC (EtOAc/PE=1:2). Work-up: the mixture was washed with water (4×100 ml), dried over Na₂SO₄ and concentrated to a red oil; giving 16.46 g of product (91% yield).
Step 2:

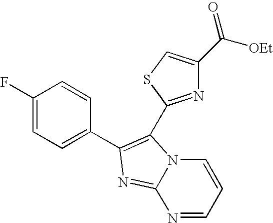

### 1-{2-|2-(4-Fluoro-phenyl)-imidazo|1,2-a|pyrimidin-3-yl|-thiazol-4-yl}-ethanone

A 250 ml round bottom flask was charged with 2-(4-Acetyl-thiazol-2-yl)-2-bromo-1-(4-fluoro-phenyl)-ethanone (5 g, 12.1 mmol), EtOH (120 ml), and pyrimidin-2-ylamine (3.45 g, 36.3 mmol). The solution was stirred overnight at room temperature. The reaction temperature was then raised to 40° C., where it stirred for an additional 24 hours. The reaction progress was monitored by TLC (1:10=MeOH/DCM). Work-up: the mixture was concentrated, dissolved in water (30 ml), extracted with Et₂O, dried over Na₂SO₄, and concentrated. The crude material was purified via column chromatography (1:50, 1:20, and 1:5=EtOAc/PE), giving yellow solid (1 g, 22% yield).
Step 3:

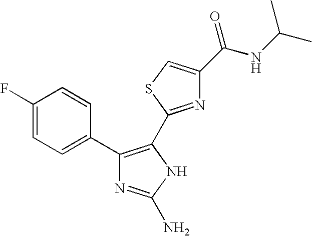

### 2-|2-Amino-5-(4-fluoro-phenyl)-3H-imidazol-4-yl|-thiazole-4-carboxylic acid isopropylamide

A 10 ml sealed tube was charged with 1-{2-[2-(4-Fluoro-phenyl)-imidazo[1,2-a]pyrimidin-3-yl]-thiazol-4-yl}-ethanone (200 mg, 0.53 mmol) and isopropylamine (5 ml). The solution was stirred at 60° C., overnight. The reaction progress was monitored by TLC (1:10=MeOH/DCM). Workup: the mixture was concentrated purified by column chromatography (1:50-1:40=MeOH/DCM), giving a yellow solid (30 mg, 16% yield) ¹H NMR (400 MHz, DMSO-d₆) δ 7.95 (m, 3H), 7.23 (m, 2H), 3.97 (m, 1H), 1.15 (d, 6H).

### EXAMPLE 97

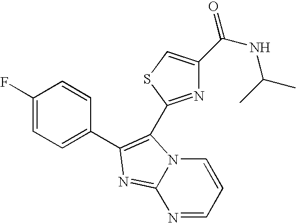

### 2-|2-(4-Fluoro-phenyl)-imidazo|1,2-a|pyrimidin-3-yl|-thiazole-4-carboxylic acid isopropylamide

Step 1:

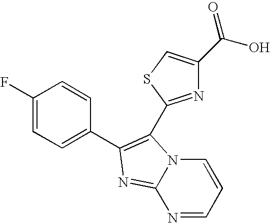

### 2-|2-(4-Fluoro-phenyl)-imidazol|1,2-a|pyrimidin-3-yl|-thiazole-4-carboxylic acid

A 50 ml round bottom flask, was charged with 1-{2-[2-(4-Fluoro-phenyl)-imidazo[1,2-a]pyrimidin-3-yl]-thiazol-4-yl}-ethanone (480 mg, 1.27 mmol), (prepared as described in step 2, of Example 96), ethanol (30 ml), and a solution of potassium hydroxide (210 mg, 3.75 mmol) dissolved in H₂O (10 ml). The solution was refluxed for 20 minutes, and the reaction progress was monitored by TLC (1:10=MeOH/DCM). The mixture was concentrated, dissolved in 30 ml of water, and acidified to pH=6 with 3M HCl (aq). A yellow solid crashed out of solution and was isolated via filtration (0.4 g, 84% yield).
Step 2:

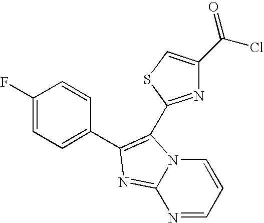

### 2-|2-(4-Fluoro-phenyl)-imidazo|1,2-a|pyrimidin-3-yl|-thiazole-4-carbonyl chloride

A 50 ml round bottom flask was charged with 2-[2-(4-Fluoro-phenyl)-imidazo[1,2-a]pyrimidin-3-yl]-thiazole-4-carboxylic acid (400 mg, 1.06 mmol), DCM (30 ml), and oxalyl chloride (1.34 g, 10.5 mmol). To this solution, DMF (two drops) was added. The reaction stirred for 2.5 hours at room temperature. Reaction progress was monitored by TLC (1:10=MeOH/DCM). The mixture was concentrated giving 0.3 g of product (71% yield), as a red solid.
Step 3:

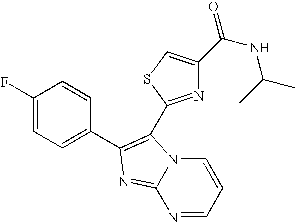

### 2-|2-(4-Fluoro-phenyl)-imidazo|1,2-a|pyrimidin-3-yl|-thiazole-4-carboxylic acid isopropylamide

A 50 ml round-bottom flask was charged with 2-[2-(4-Fluoro-phenyl)-imidazo[1,2-a]pyrimidin-3-yl]-thiazole-4-carbonyl chloride (300 mg, 0.75 mmol), DCM (30 ml), and isopropylamine (2 ml). The reaction stirred at room temperature for 0.5 hours, and the progress was monitored by TLC (1:10=MeOH/DCM). The mixture was washed with water; extracted with DCM dried over MgSO_{4,} and concentrated. The crude material was purified via column chromatography (1:100, 1:200=MeOH/DCM), giving-120 mg of a yellow solid (38% yield). ¹H NMR (400 MHz, CDCl₃) δ: 9.52 (bs, 1H), 8.78 (bs, 1H), 8.10 (s, 1H), 7.75 (m, 2H), 7.16 (s, 4H), 6.98 (bs, 1H), 4.35 (m, 1H), 3.84 (m, 1H), 11.35 (d, 6H), 1H) (d,3H), 1.20 (d, 3H), 0.88 (m, 1H).

### EXAMPLE 2158

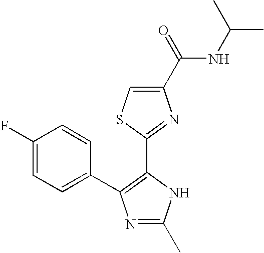

### 2-|5-(4-Fluoro-phenyl)-2-methyl-3H-imidazol-4-yl|-thiazole-4-carboxylic acid isopropylamide

A 10 ml sealed tube was charged with 2-[3-(4-Fluoro-phenyl)-5-methyl-isoxazol-4-yl]-thiazole-4-carboxylic acid ethyl ester (600 mg, 1.02 mmol, described in step 9 of example 80) and isopropylamine (5 ml). The resulting solution was stirred for 6 hours at 50° C. Reaction progress was monitored by TLC (CH₂Cl₂/MeOH=10:1). Work-up: the mixture was concentrated and purified by column chromatography with a 40:1 CH₂Cl₂/MeOH, yielding 80 mg (21%) of product as a light yellow solid. ¹H NMR (400 MHz, CDCl₃) δ: 7.96 (s, 1H), 7.70 (m, 2H), 7.17 (m, 2H), 4.25 (m, 1H), 2.74 (s, 3H), 1.19 (d, 6H).

### EXAMPLE 2159

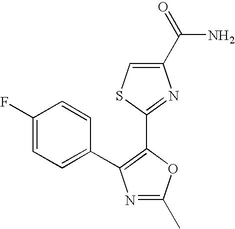

### 2-|4-(4-Fluoro-phenyl)-2-methyl-oxazol-5-yl|-thiazole-4-carboxylic acid amide

The title compound was prepared analogously to 2-[5-(4-Fluoro-phenyl)-2-morpholin-4-ylmethyl-3H-imidazol-4-yl]-thiazole-4-carboxylic acid isopropylamide (example 38), where ammonium hydroxide was substituted for isopropylamine in step 7 of that sequence. ¹H NMR (400 MHz, Acetone d₆) δ: 8.42 (m, 2H), 8.32 (s, 1H), 7.27 (m, 2H), 2.59 (s, 3H).

### EXAMPLE 2160

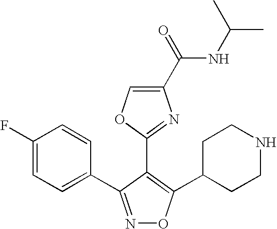

### 2-|3-(4-Fluoro-phenyl)-5-piperidin-4-yl-isoxazol-4-yl|-oxazole-4-carboxylic acid isopropylamide

Step 1

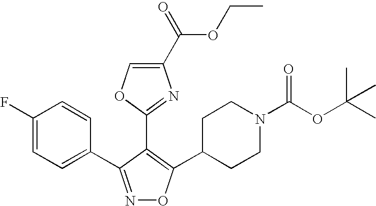

### 4-|3-(4-Fluoro-phenyl)-4-(4-isopropylcarbamoyl-oxazol-2-yl)-isoxazol-5-yl|-piperidine-1-carboxylic acid tert-butyl ester

A flask equipped with a reflux condensor was charged with ethyl 4-ethynyl-piperidine-1-carboxylic acid tert-butyl ester (174 mg, 1.0 mmol, prepared as described in J. Med. Chem. 2004, 47, 3111-3130), 4-Fluoro-benzaldehyde chloro-oxime (347 mg, 1.0 mmol, prepared as described in step 2 of example 27), and Et₃N/Et₂O (1:3 v/v, 5 mL). The resulting solution was stirred for 24 hours at 50° C. Reaction progress was monitored by LCMS. Work-up: the mixture was concentrated, dissolved in DMF, and purified by RPHPLC, giving 70 mg of the title compound, 6% yield.
Step 2

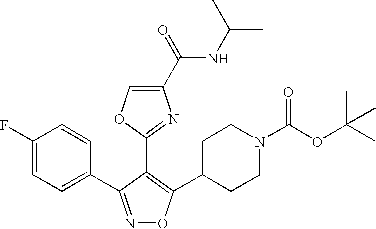

### 4-|3-(4-Fluoro-phenyl)-4-(4-isopropylcarbamoyl-oxazol-2-yl)-isoxazol-5-yl|-piperidine-1-carboxylic acid tert-butyl ester

A vial was charged with 4-[3-(4-Fluoro-phenyl)-4-(4-isopropylcarbamoyl-oxazol-2-yl)-isoxazol-5-yl]-piperidine-1-carboxylic acid tert-butyl ester (70 mg, 0.144 mmol), MeAlCl(NH-ispropyl) (430 μL of 0.67 M solution, prepared as described in Synthetic Communications, 12 (13), 989-993 (1982)), and toluene (430 μL). The resulting solution stirred for 1.5 hours at 80° C. Reaction progress was monitored by HPLC. Work-up: the reaction was diluted with DCM (20 mL), and stirred with Na₂SO₄-10H₂O (1 g) for 1 hr, then filtered, and concentrated to give 66 mg of crude product, which was used in the next step without further purification.
Step 3

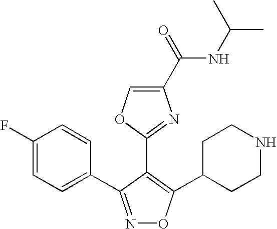

### 2-|3-(4-Fluoro-phenyl)-5-piperidin-4-yl-isoxazol-4-yl|-oxazole-4-carboxylic acid isopropylamide

A flask was charged with 4-[3-(4-Fluoro-phenyl)-4-(4-isopropylcarbamoyl-oxazol-2-yl)-isoxazol-5-yl]-piperidine-1-carboxylic acid tert-butyl ester (66 mg, 0.132 mmol), and 30% TFA/CH₂Cl₂ (3 mL). The resulting solution was stirred for 1 hour at room temperature. Reaction progress was monitored by LCMS. Work-up: the mixture was concentrated, dissolved in DMF, and purified by RPHPLC, giving 30 mg of the title compound as a pale yellow oil, 52% yield for two steps. ¹H NMR (400 MHz, CDCl₃) δ: 9.28 (bs, 1H), 8.96 (bs, 1H), 8.27 (s, 1H), 7.59 (m, 2H), 7.18 (m, 2H), 6.65 (d, 2H), 6.09 (bs, 2H), 4.21 (m, 1H), 3.75 (bm, 1H), 3.65 (bm, 2H), 3.23 (bm, 2H), 2.32 (bm, 4H), 1.25 (d, 6H).

### EXAMPLE 2161

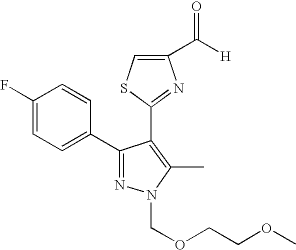

### 2-|3-(4-Fluoro-phenyl)-1-(2-methoxy-ethoxymethyl)-5-methyl-1H-pyrazol-4-yl|-thiazole-4-carbaldehyde

Step 1

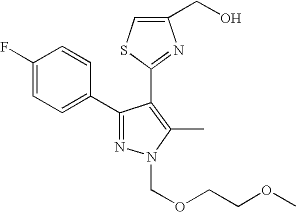

### {2-|3-(4-Fluoro-phenyl)-1-(2-methoxy-ethoxymethyl)-5-methyl-1H-pyrazol-4-yl|-thiazol-4-yl}-methanol

A 100 ml roundbottom flask was charged with 2-[3-(4-Fluoro-phenyl)-1-(2-methoxy-ethoxymethyl)-5-methyl-1H-pyrazol-4-yl]-thiazole-4-carboxylic acid ethyl ester (10.3 g, 24.6 mmol, prepared as described in step 8 of example 79) and THF (80 ml). The resulting solution was cooled to 0° C., and LiAlH₄ (1.87 g, 49.2 mmol) was added in several batches. The resulting solution was stirred for 1 hour d at room temperature. Reaction progress was monitored by TLC (CH₂Cl₂/MeOH=15:1). Work-up: the reaction mixture was diluted with 50 ml of H₂O/ice, extracted with 100 ml of EtOAc, dried over Na₂SO₄, concentrated to a yellow oil (9.28 g), and used without further purification.
Step 2

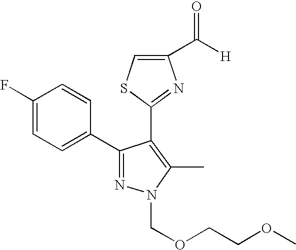

### 2-|3-(4-Fluoro-phenyl)-1-(2-methoxy-ethoxymethyl)-5-methyl-1H-pyrazol-4-yl|-thiazole-4-carbaldehyde

A round bottom flask was charged with {2-[3-(4-Fluoro-phenyl)-1-(2-methoxy-ethoxymethyl)-5-methyl-1H-pyrazol-4-yl]-thiazol-4-yl}-methanol (9.28 g, 24.62 mmol), CHCl₃ (100 ml), and PCC (10.61 g, 49.22 mmol) in several batches. The resulting solution was stirred overnight at room temperature. Reaction progress was monitored by TLC (CH₂Cl₂/MeOH=15:1). The residue was purified by column chromatography with a 1000:1 CH₂Cl₂/MeOH solvent system, resulting in 4.2 g (45.7%) of the title compound as yellow-green oil. ¹H NMR (400 MHz, CDCl₃) δ: 10.03 (s, 1H), 7.93 (s, 1H), 7.18 (m, 3H), 5.28 (s, 2H), 3.94 (s, 2H), 3.50 (s, 2H), 3.33 (s, 3H), 2.63 (s, 3H).

### EXAMPLE 2162

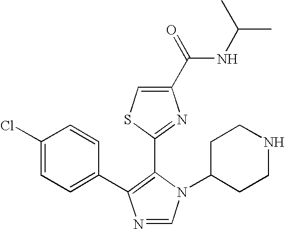

### 2-|5-(4-Chloro-phenyl)-3-piperidin-4-yl-3H-imidazol-4-yl|-thiazole-4-carboxylic acid isopropylamide

The title compound was prepared analogously to 2-[5-(4-Fluoro-phenyl)-3-piperidin-4-yl-3H-imidazol-4-yl]-thiazole-4-carboxylic acid isopropylamide, example 91. ¹H NMR (400 MHz, d₄-Methanol) δ: 8.41 (s, 1H), 8.31 (s, 1H), 7.43 (s, 4H), 4.87 (s, 2H), 4.24 (m, 1H), 3.58 (d, 2H), 3.33 (d, 2H), 3.18 (t, 2H), 2.50 (d, 2H), 2.24 (m, 2H), 1.24 (d, 6H).

### EXAMPLE 2163

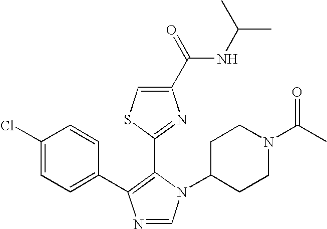

### 2-|5-(4-Chloro-phenyl)-3-piperidin-4-yl-3H-imidazol-4-yl|-thiazole-4-carboxylic acid isopropylamide

A round bottom flask was charged with 2-[5-(4-Chloro-phenyl)-3-piperidin-4-yl-3H-imidazol-4-yl]-thiazole-4-carboxylic acid isopropylamide (43 mg, 0.065 mmol), DMF (0.5 mL), acetic anhydride (12.3 μL, 0.13 mmol) and Et₃N (36 μL, 0.26 mmol). The resulting solution was stirred overnight at room temperature. Work-up: the crude reaction was purified by RPHPLC, giving the title compound as a white solid (30 mg, 84%). ¹H NMR (400 MHz, d₄-Methanol) δ: 8.70 (s, 1H), 8.34 (s, 1H), 7.44 (s, 4H), 4.88 (s, 2H), 4.70 (m, 1H), 4.23 (m, 1H), 4.07 (m, 1H), 3.25 (m, 1H), 2.70 (m, 1H), 2.27 (m, 2H), 2.14 (s, 1H), 1.96 (bm, 2H), 1.20 (d, 6H).

### EXAMPLE 2164

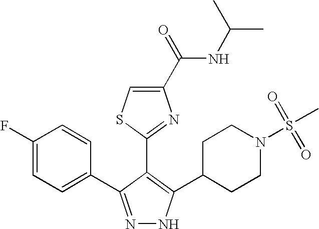

### 2-|3-(4-Fluoro-phenyl)-5-(1-methanesulfonyl-piperidin-4-yl)-1H-pyrazol-4-yl|-thiazole-4-carboxylic acid isopropylamide

A round bottom flask was charged with 2-[3-(4-Fluoro-phenyl)-5-piperidin-4-yl-1H-pyrazol-4-yl]-thiazole-4-carboxylic acid isopropylamide (67.5 mg, 0.15 mmol, described in example 61), NMP (0.5 mL), MeSO₂Cl (17.5 μL, 0.23 mmol) and N-methylmorpholine (49.5 μL, 0.45 mmol). The resulting solution was at room temperature. LCMS after 30 min showed bis acylation. The reaction was treated with NaOH (aqueous syrup, 250 μL). LCMS after 30 min shows mainly product. Work-up: the reaction was acidified with 1N HCl, extracted with CH₂Cl₂ (10×5 mL), concentrated, and purified by RPHPLC, giving the title compound (30 mg, 39%). ¹H NMR (400 MHz, d₄-Methanol) δ: 8.70 (s, 1H), 8.34 (s, 1H), 7.44 (s, 4H), 4.88 (s, 2H), 4.70 (m, 1H), 4.23 (m, 1H), 4.07 (m, 1H), 3.25 (m, 1H), 2.70 (m, 1H), 2.27 (m, 2H), 2.14 (s, 1H), 1.96 (bm, 2H), 1.20 (d, 6H).

### EXAMPLE 2165

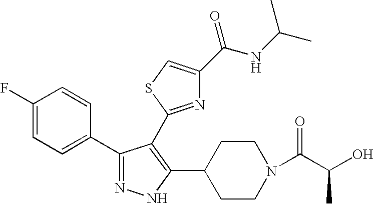

### 2-{3-(4-Fluoro-phenyl)-5-|1-(2-hydroxy-propionyl)-piperidin-4-yl|-1H-pyrazol-4-yl}-thiazole-4-carboxylic acid isopropylamide

The title compound was prepared analogously to Example 62, where (s)-(−)acetic acid-1-chlorocarbonyl-ethyl ester (42.0 μL) was substituted for acetoxy acetyl hydrochloride. (98 mg, 67% yield, calculated as TFA salt). ¹H NMR (400 MHz, d6-dmso): δ 8.13 (s, 1H), 7.62 (d, 1H), 7.51 (m, 2H), 7.28 (m, 2H), 4.53 (m, 3H), 4.06 (m, 4H), 3.40 (m, 1H), 3.07 (m, 1H), 2.66 (m, 1H), 2.49 (m, 1H), 1.93 (m, 3H), 1.65 (m, 3H), 1.20 (m, 3H), 1.15 (d, 6H). LCMS: 487.05 (M+1)⁺.

### EXAMPLE 2166

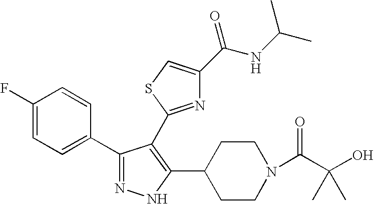

### 2-{3-(4-Fluoro-phenyl)-5-|1-(2-hydroxy-2-methyl-propionyl)-piperidin-4-yl|-1H-pyrazol-4-yl}-thiazole-4-carboxylic acid isopropylamide

The title compound was prepared analogously to Example 62, where acetic acid-1-chlorocarbonyl-1-methyl-ethyl ester (48.0 μL) was substituted for acetoxy acetyl hydrochloride. (94.7 mg, 65% yield, calculated as TFA salt). ¹H NMR (400 MHz, d6-dmso): δ 8.13 (s, 1H), 7.61 (d, 1H), 7.48 (m, 2H), 7.26 (m, 2H), 4.81 (m, 1H), 4.45 (m, 1H), 4.03 (m, 2H), 3.37 (m, 2H), 2.95 (m, 1H), 2.42 (m, 1H), 1.92 (m, 3H), 1.64 (m, 3H), 1.31 (d, 6H), 1.15 (d, 6H). LCMS: 500.71 (M+1)⁺.

### EXAMPLE 2167

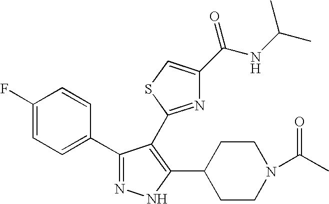

### 2-|5-(1-Acetyl-piperidin-4-yl)-3-(4-fluoro-phenyl)-1H-pyrazol-4-yl|-thiazole-4-carboxylic acid isopropylamide

A 100 mL flask was charged with 2-[3-(4-Fluoro-phenyl)-5-piperidin-4-yl-1H-pyrazol-4-yl]-thiazole-4-carboxylic acid isopropylamide (100.0 mg, 0.24 mmol, prepared as described in step 10 of Example 47), DCM (0.8 mL), Et₃N (0.17 mL, 1.2 mmol), and acetic anhydride (22.7 μL). The solution was stirred for 15 minutes at room temperature, and reaction progress was monitored by LC/MS. Work-up: the crude mixture was diluted with EtOAc, washed with water, dried over MgSO₄, and concentrated to yield a solid. This material was purified via HPLC to yield 55.0 mg of product (50% yield, calculated as TFA salt). ¹H NMR (400 MHz, d6-dmso): 8.13 (s, 1H), 7.61 (m, 2H), 7.50 (m, 2H), 7.26 (d, 3H), 4.48 (m, 1H), 3.87 (m, 1H), 3.36 (m, 2H), 2.42 (m, 1H), 2.01 (s, 3H), 1.90 (m, 2H), 1.15 (d, 6H). LCMS: 457.13 (M+1)⁺.

### EXAMPLE 2168

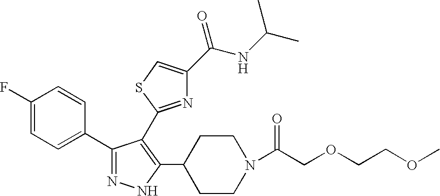

### 2-(3-(4-Fluoro-phenyl)-5-{1-|2-(2-methoxy-ethoxy)-acetyl|-piperidin-4-yl}-1H-pyrazol-4-yl)-thiazole-4-carboxylic acid isopropylamide

The title compound was prepared analogously to Example 62, where (2-Methoxy-ethoxy)-acetyl chloride (36.7 μL) was substituted for acetic anhydride, yielding 55.7 mg of product (53% yield, calculated as TFA salt). ¹H NMR (400 MHz, d6-dmso): δ 8.13 (s, 1H), 7.68 (m, 2H), 7.49 (m, 2H), 7.26 (m, 3H), 4.18 (m, 2H), 4.12 (m, 4H), 3.58 (m, 1H), 3.48(m, 2H), 3.24 (s, 3H), 2.67 (m, 2H), 2.37 (m, 2H), 1.91 (m, 4H), 1.78 (m, 1H), 1.18 (d, 6H). LCMS: 531.79 (M+1)⁺.

### EXAMPLE 2169

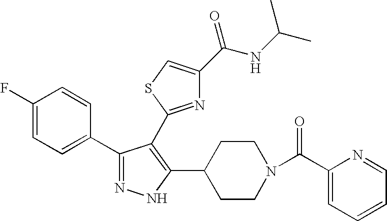

### 2-{3-(4-Fluoro-phenyl)-5-|1-(pyridine-2-carbonyl)-piperidin-4-yl|-1H-pyrazol-4-yl}-thiazole-4-carboxylic acid isopropylamide

The title compound was prepared analogously to Example 62 where pyridine-2-carbonyl chloride HCl salt (43.0 mg) was substituted for acetic anhydride, yielding 38.0 mg of product (34% yield, calculated as TFA salt). ¹H NMR (400 MHz, d6-dmso): δ 8.58 (d, 1H), 8.13 (s, 1H), 7.92 (m, 1H), 7.61 (m, 1H), 7.54 (d, 2H), 7.47 (m, 2H), 7.25 (m, 2H), 4.63 (m, 1H), 4.03 (m, 2H), 3.72 (m, 2H), 3.13 (m, 1H), 2.89 (m, 1H), 2.76 (m, 1H), 2.12 (m, 1H), 1.85 (m, 1H), 1.77 (m, 1H), 1.15 (d, 6H). LCMS: 520.85 (M+1)⁺.

### EXAMPLE 2170

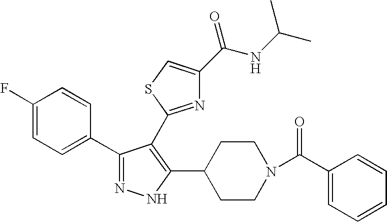

### 2-|5-(1-Benzoyl-piperidin-4-yl)-3-(4-fluoro-phenyl)-1H-pyrazol-4-yl|-thiazole-4-carboxylic acid isopropylamide

The title compound was prepared analogously to Example 62 where benzoyl chloride (26 μL) was substituted for acetic anhydride, yielding 60.0 mg of product (59% yield, calculated as TFA salt). ¹H NMR (400 MHz, d6-dmso): δ 8.14 (s, 1H), 7.62 (m, 1H), 7.49 (m, 3H), 7.43 (d, 4H), 7.39 (m, 3H), 7.23 (m, 2H), 4.03 (m, 1H), 3.67 (m, 1H), 3.37 (s, 1H), 2.37 (m, 1H), 2.05 (m, 1H) 1.78 (m, 1H), 1.15 (d, 6H). LCMS: 518.87 (M+1)⁺.

### EXAMPLE 2171

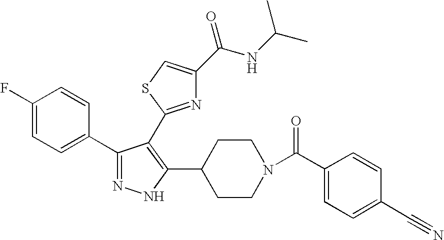

### 2-|5-|1-(4-Cyano-benzoyl)-piperidin-4-yl|-3-(4-fluoro-phenyl)-1H-pyrazol-4-yl|-thiazole-4-carboxylic acid isopropylamide

The title compound was prepared analogously to Example 62 where 4-cyano benzoyl chloride (44.0 mg) was substituted for acetic anhydride, yielding 72.6 mg of product (55% yield, calculated as TFA salt). ¹H NMR (400 MHz, d6-dmso): δ 8.14 (s, 1H), 7.93 (d, 2H), 7.63 (d, 1H), 7.58 (d, 2H), 7.50 (d, 2H), 7.26 (m, 2H), 4.57 (m, 1H), 4.03 (m, 1H), 3.52 (m, 1H), 3.44 (m, 1H), 3.32 (m, 1H), 2.92 (m, 1H), 2.03 (m, 1H), 1.83 (m, 1H), 1.76 (m, 1H), 1.15 (d, 6H). LCMS: 543.44 (M+1)⁺.

### EXAMPLE 2172

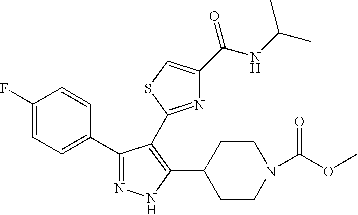

### 4-|5-(4-Fluoro-phenyl)-4-(4-isopropyl-carbamoyl-thiazol-2-yl)-2H-pyrazol-3-yl|-piperidine-1-carboxylic acid methyl ester

The title compound was prepared analogously to Example 62, where methyl chloroformate (17.6 μL, 0.24 mmol) was substituted for acetic anhydride (60 mg, 59% yield, calculated as TFA salt). ¹H NMR (400 MHz, d6-dmso): δ 8.13 (s, 1H), 7.60 (d, 1H), 7.49 (m, 2H), 7.23 (m, 2H), 4.04 (m, 3H), 3.60 (s, 3H), 3.31 (m, 1H), 2.87 (m, 2H), 1.89 (m, 2H), 1.66 (m, 2H), 1.15 (d, 6H). LCMS: 472.07 (M+1)⁺.

### EXAMPLE 2173

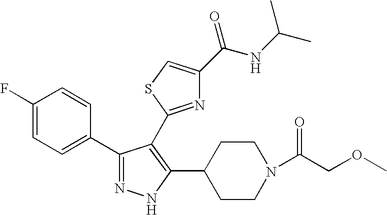

### 2-{3-(4-Fluoro-phenyl)-5-|1-(2-methoxy-acetyl)-piperidin-4-yl|-1H-pyrazol-4-yl}-thiazole-4-carboxylic acid isopropylamide

The title compound was prepared analogously to Example 62, where methoxy-acetyl chloride (20.0 μL) was substituted for acetic anhydride, yielding 50.3 mg of product (51% yield, calculated as TFA salt). ¹H NMR (400 MHz, d6-dmso): δ 8.13 (s, 1H), 7.61 (m, 2H), 7.50 (m, 4H), 7.25 (d, 3H), 4.43 (m,2H), 4.11 (m, 2H), 4.03 (m, 4H), 3.84 (m, 2H), 3.27 (s, 3H), 3.06 (m, 2H), 2.64 (m, 2H), 2.39 (m, 1H), 1.91 (m, 4H), 1.60 (m, 3H), 1.16 (d, 6H). LCMS: 487.11 (M+1)⁺.

### EXAMPLE 2174

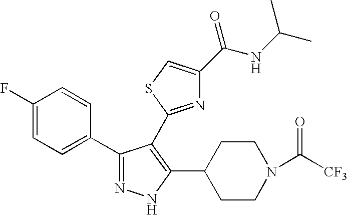

### 2-{3-(4-Fluoro-phenyl)-5-|1-(2,2,2-trifluoro-acetyl)-piperidin-4-yl|-1H-pyrazol-4-yl}-thiazole-4-carboxylic acid isopropylamide

The title compound was prepared analogously to Example 62, where TFA anhydride (31.0 μL) was substituted for acetic anhydride, yielding 60 mg of product. (59% yield, calculated as TFA salt). ¹H NMR (400 MHz, d6-dmso): δ 8.13 (s, 1H), 7.62 (m, 2H), 7.49 (m, 2H), 7.28 (d, 3H), 4.48 (m, 1H), 3.87 (m, 1H), 3.36 (m, 2H), 2.42 (m, 1H), 1.91 (m, 2H), 1.16 (d, 6H). LCMS: 511.07 (M+1)⁺.

### EXAMPLE 2175

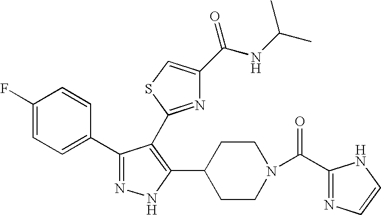

### 2-{3-(4-Fluoro-phenyl)-5-|1-(1H-imidazole-2-carbonyl)-piperidin-4-yl|-1H-pyrazol-4-yl}-thiazole-4-carboxylic acid isopropylamide

An 8 ml vial was charged with 2-[3-(4-Fluoro-phenyl)-5-piperidin-4-yl-1H-pyrazol-4-yl]-thiazole-4-carboxylic acid isopropylamide (82.6 mg, 0.20 mmol), DCM (0.8 ml), DIEA (0.17 ml, 1.0 mmol), EDC (46 mg, 0.24 mmol), and 1-imidizole-2-carboxylic acid. The solution was stirred at room temperature for 3 days, and was monitored by LC/MS. Workup: the crude mixture was diluted with EtOAc, and was washed once with 1 M HCL (50 ml). The aqueous phase was washed twice with ethyl acetate, and the organics were combined, dried over MgSO₄, and concentrated. The title compound was purified via reverse phase HPLC to yield 24.3 mg of product (24%, calculated as TFA salt). ¹H NMR (400 MHz, d6-dmso): δ 8.30 (s, 1H), 8.15 (s, 1H), 7.62 (m, 2H), 7.50 (m, 2H), 7.25 (m, 2H), 7.20 (m, 1H), 4.03 (m, 1H), 3.67 (m, 2H), 2.48 (m, 1H), 2.01 (m, 1H), 1.15 (d, 6H). LCMS: 518.87 (M+1)⁺.

### EXAMPLE 2176

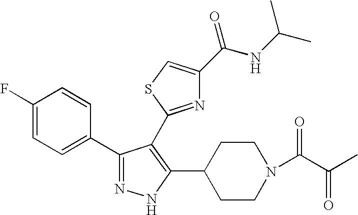

### 2-{3-(4-Fluoro-phenyl)-5-|1-(2-oxo-propionyl)-piperidin-4-yl|-1H-pyrazol-4-yl}-thiazole-4-carboxylic acid isopropylamide

The title compound was prepared analogously to Example 62, where 2-oxo-propionic acid (16.7 μL) was substituted for 1H-imidizole-2-carboxylic acid. The compound was purified via reverse phase HPLC to yield 20.1 mg of product (22%, calculated as TFA salt). ¹H NMR (400 MHz, d6-dmso): δ 8.14 (s, 1H), 7.65 (m, 2H), 7.50 (m, 3H), 7.26 (m, 3H), 4.32 (m, 2H), 4.03 (m, 2H), 3.66 (m, 2H), 2.80 (m, 2H), 2.37 (s, 3H), 1.94 (m, 3H), 1.78 (m, 1H), 1.67 (m, 2H), 1.15 (d, 6H). LCMS: 484.45 (M+1)⁺.

### EXAMPLE 2177

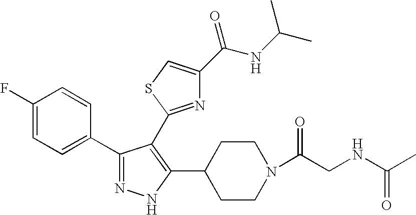

### 2-|5-|1-(2-Acetylamino-acetyl)-piperidin-4-yl|-3-(4-fluoro-phenyl)-1H-pyrazol-4-yl|-thiazole-4-carboxylic acid isopropylamide

The title compound was prepared analogously to Example 62, where acetylamino-acetic acid (28.1 mg) was substituted for 1H-imidizole-2-carboxylic acid. The title compound was purified via reverse phase HPLC to yield 56.5 mg of product (58% yield, calculated as TFA salt). ¹H NMR (400 MHz, d6-dmso): δ 8.13 (s, 1H), 7.95 (t, 1H), 7.61 (d, 1H), 7.51 (m, 2H), 7.26 (m, 2H), 4.46 (d, 1H), 4.07 (m, 1H), 3.93 (m, 2H), 3.88 (m, 1H), 3.37 (m, 1H), 3.09 (m, 1H), 2.66 (m, 1H), 1.94 (d, 1H), 1.86 (s, 3H), 1.74 (m, 1H), 1.60 (m, 1H), 1.15 (d, 6H). LCMS: 513.65 (M+1)⁺.

### EXAMPLE 2178

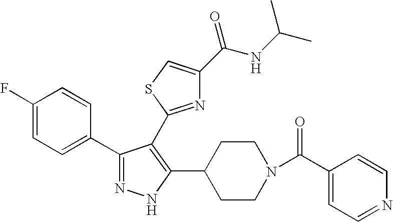

### 2-{3-(4-Fluoro-phenyl)-5-|1-(pyridine-4-carbonyl)-piperidin-4-yl|-1H-pyrazol-4-yl}-thiazole-4-carboxylic acid isopropylamide

The title compound was prepared analogously to Example 62, where isonicotinic acid (14.8 mg) was substituted for 1H-imidizole-2-carboxylic acid. The title compound was purified via reverse phase HPLC to yield 34.8 mg of product (% yield, calculated as TFA salt). ¹H NMR (400 MHz, d6-dmso): δ 8.76 (m, 3H), 8.13 (s, 1H), 7.64 (d, 1H), 7.57 (m, 3H), 7.49 (m, 3H), 7.25 (m, 3H), 4.56 (d, 2H), 4.04 (m, 2H), 3.46 (m, 3H), 3.19 (m, 1H), 2.91 (m, 1H), 2.36 (m, 1H), 2.05 (m, 1H), 1.84 (m, 1H), 1.76 (m, 2H), 1.15 (d, 6H). LCMS: 520.85 (M+1)⁺.

### EXAMPLE 2179

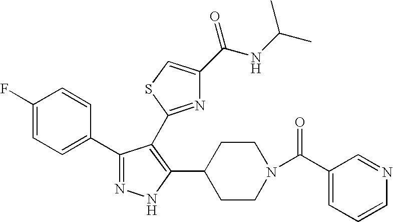

### 2-{3-(4-Fluoro-phenyl)-5-|1-(pyridine-3-carbonyl)-piperidin-4-yl|-1H-pyrazol-4-yl}-thiazole-4-carboxylic acid isopropylamide

The title compound was prepared analogously to Example 62, where nicotinic acid (123 mg) was substituted for 1H-imidazole-2-carboxylic acid. The compound was purified via reverse phase HPLC to yield 64 mg of product (58% yield, calculated as TFA salt). ¹H NMR (400 MHz, d6-dmso): δ 9.47 (m, 2H), 8.95 (s, 1H), 8.70 (d, 1H), 8.44 (d, 1H), 8.35 (m, 1H), 8.31 (m, 2H), 8.07 (m, 2H), 5.39 (m,2H), 4.82 (m, 1H), 4.44 (m, 2H), 4.24(m, 1H), 4.03 (m, 1H), 3.67 (m, 1H), 3.33 (s, 1H), 2.86 (m, 1H), 2.68 (m, 1H), 2.57 (m, 2H), 1.96 (d, 6H). LCMS: 520.89 (M+1)⁺.

### EXAMPLE 2180

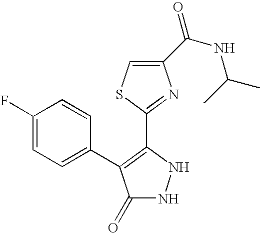

### 2-|4-(4-Fluoro-phenyl)-5-oxo-2,5-dihydro-1H-pyrazol-3-yl|-thiazole-4-carboxylic acid isopropylamide

Step 1

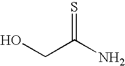

### 2-Hydroxythioacetamide

A 1000 mL 3-necked round bottom flask was charged with hydroxy-acetonitrile (100 g, 964.91 mmol). To this was added H₂S (32.8 g, 964.71 mmol). The resulting mixture was allowed to stir at room temperature overnight. The mixture was then concentrated in vacuo to afford 84 g (96%) of 2-hydroxythioacetamide as yellow solid. This product was used without further purification.
Step 2

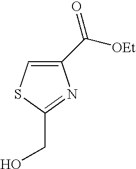

### 2-Hydroxymethylthiazole-4-carboxylic acid ethyl ester

A 500 mL round bottom flask was charged with a solution of 2-hydroxythioacetamide (30 g 263.74 mmol) in EtOH (150 mL). To this mixture was added 3-bromo-2-oxopropionic acid ethyl ester (96 g, 492.31 mmol). The resulting solution was allowed to reflux for 1.5 h. The reaction progress was monitored by TLC (CH₂Cl₂:MeOH=10:1). The mixture was the concentrated in vacuo to give a residue that was purified by a column chromatography eluted with a 100:1 CH₂Cl₂/MeOH affording 35 g (64%) of 2-hydroxymethyl thiazole-4-carboxylic acid ethyl ester a pale yellow solid. MS: 188 [M+H]⁺
Step 3

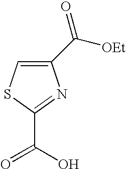

### Thiazole-2,4-dicarboxylic acid 4-ethyl ester

A 250 ml round bottom flask was charged with a solution of 2-hydroxymethyl thiazole-4-carboxylic acid ethyl ester (5 g, 26.74 mmol) in water (20 mL) followed by the addition 150 mL aqueous solution of KMnO4 (7.0 g, 44.30 mmol) drop wise at room temperature over 4 hours. The reaction progress was monitored by TLC (CH₂Cl₂:CH₃OH=8:1). The pH was adjusted to 8-9 by the addition of K₂CO₃. The mixture was filtered off and washed with water (2×20 mL). The filtrate was extracted with CHCl₃ (5×30 mL). The combined aqueous layers was added conc. HCl to adjust the pH to 2-3. The resulting solution was extracted with CHCl₃ (5×30 mL), the combined organic layers were concentrated in vacuo to afford 3.6 g (67%) of thiazole-2,4-dicarboxylic acid 4-ethyl ester as a white solid. This was used without further purification.
Step 4

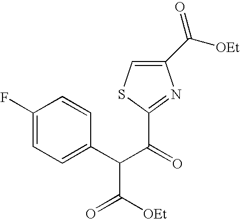

### 2-|2-Ethoxycarbonyl-2-(4-fluoro-phenyl)-acetyl|-thiazole-4-carboxylic acid ethyl ester

A 250 mL round bottom flask was charged with a solution of thiazole-2,4-dicarboxylic acid 4-ethyl ester (2.01 g, 10.00 mmol) in DMF (40 mL). To this was added carbonyldiimidazole (1.8 g, 11.25 mmol). The mixture was heated to 80-90° C. The disappearance of starting material was monitored by TLC (CH₂Cl₂:CH₃OH=10:1). Then, the flask was cooled down to room temperature. To this was added ethyl 2-(4-fluorophenyl)acetate (2.0 g, 10.99 mmol) followed by addition of NaH (1.4 g, 35.00 mmol) at −25° C. The resulting solution was allowed stir at this temperature for 15 min. Then, the flask was warned up to room temperature and allowed stir for 2 hours. The reaction progress was monitored by TLC (EtOAc:PE=1:4). The mixture was quenched by 100 mL ice water. The pH was adjusted to 5-6 with conc. HCl. The mixture was then rinsed into a separatory funnel and extracted with EtOAc (3×100 mL) and dried over Na₂SO₄. The combined organic layers were concentrated in vacuo to afford a residue that was purified by a column chromatography eluted with EtOAc/PE=1:20. This resulted in 2.1 g (57%) of 2-[2-ethoxycarbonyl-2-(4-fluoro-phenyl)-acetyl]-thiazole-4-carboxylic acid ethyl ester as a white solid. This was used without further purification. MS: 366 [M+H]⁺
Step 5

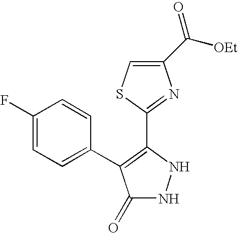

### 2-|4-(4-Fluoro-phenyl)-5-oxo-2,5-dihydro-1H-pyrazol-3-yl|-thiazole-4-carboxylic acid ethyl ester

A 50 mL round bottom flask was charged with solution of 2-[2-ethoxycarbonyl-2-(4-fluoro-phenyl)-acetyl]-thiazole-4-carboxylic acid ethyl ester (100 mg, 0.27 mmol) in AcOH (10 mL). To the mixture was added hydrazine hydrate (40 mg, 0.64 mmol). The resulting solution was allowed to reflux until the progress of the reaction was monitored by TLC (CH₂Cl₂:CH₃OH=15:1). The mixture was concentrated in vacuo. The final product was purified by recrystallized from EtOAc to afford 60 mg (66%) of 2-[4-(4-fluoro-phenyl)-5-oxo-2,5-dihydro-1H-pyrazol-3-yl]-thiazole-4-carboxylic acid ethyl ester as a white solid. This was used without further purification.
Step 6

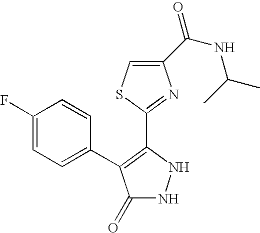

### 2-|4-(4-Fluoro-phenyl)-5-oxo-2,5-dihydro-1H-pyrazol-3-yl|-thiazole-4-carboxylic acid isopropyl amide

A 10 mL sealed tube was charged with 2-[4-(4-fluoro-phenyl)-5-oxo-2,5-dihydro-1H-pyrazol-3-yl]-thiazole-4-carboxylic acid ethyl ester (160 mg, 0.48 mmol). To this was added propan-2-amine (6 mL). The resulting solution was allowed to reflux for 48 h. The reaction progress was monitored by TLC (CH₂Cl₂:CH₃OH=10:1). The mixture was concentrated in vacuo to afford a residue that was purified by column chromatography eluted with a 99:1 CH₂Cl₂/MeOH. This resulted in 42.4 mg (26%) of 2-[4-(4-Fluoro-phenyl)-5-oxo-2,5-dihydro-1H-pyrazol-3-yl]-thiazole-4-carboxylic acid isopropyl amide as a white solid. 1H NMR (400 MHz, DMSO) δ: 12.70 (s, 1H), 8.12 (s, 1H), 7.46 (d, 2H), 7.22 (d, 2H), 6.89 (d, 1H,), 4.01 (1H,s), 3.96 (q, 1H), 1.11 (d, 6H). MS: 347.0 [M+H]⁺.

### EXAMPLE 2181

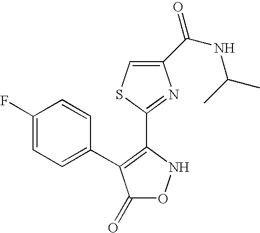

### 2-|4-(4-Fluoro-phenyl)-5-oxo-2,5-dihydro-isoxazol-3-yl|-thiazole-4-carboxylic acid isopropyl amide

The title compound was prepared analogously to 2-[4-(4-Fluoro-phenyl)-5-oxo-2,5-dihydro-1H-pyrazol-3-yl]-thiazole-4-carboxylic acid isopropylamide, Example 2180, where hydroxylamine hydrochloride was substituted for hydrazine in step 4 of that example. 1H NMR (400 MHz, DMSO) δ: 10.94 (s, 1H), 8.08 (s, 1H), 7.50-7.46 (m, 2H), 7.22-7.16 (m, 2H), 6.81 (d, 1H,), 3.94-3.87 (m, 1H), 3.66 (s, 3H), 3.31 (br, s, 1H), 1.06 (d, 6H). MS: 359.10 [M+H]⁺.

### EXAMPLE 2182

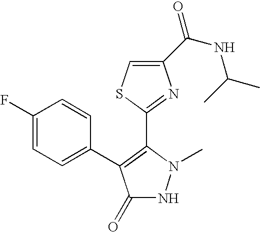

### 2-|4-(4-Fluoro-phenyl)-2-methyl-5-oxo-2,5-dihydro-1H-pyrazol-3-yl|-thiazole-4-carboxylic acid isopropyl amide

Step 1

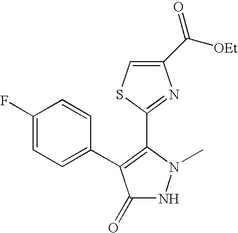

### 2-|4-(4-fluoro-phenyl)-2-methyl-5-oxo-2,5-dihydro-1H-pyrazol-3-yl|-thiazole-4-carboxylic acid ethyl

A 50 ml 3-necked round bottom flask was charged with a solution of 2-[2-ethoxycarbonyl-2-(4-fluoro-phenyl)-acetyl]-thiazole-4-carboxylic acid ethyl ester (250 mg, 0.65 mmol, described in step 4 of Example 2180 in EtOH (15 ml) followed by the addition of CH₃NHNHBoc (200 mg, 1.38 mmol) in EtOH (5 ml) drop wise at −20° C. The resulting solution was allowed to stir at room temperature for 5 hours. To the mixture was added 4-methylbenzenesulfonic acid (50 mg, 0.29 mmol) and allowed to reflux for overnight. The reaction progress was monitored by TLC (CH₂Cl₂/MeOH=15:1). The mixture was concentrated in vacuo to afford 60 mg (25%) of the title compound as a yellow solid. ¹H NMR (400 MHz, DMSO) δ: 8.39 (s, H), 7.59 (t, 2H), 7.13 (t, 2H), 4.23 (q, 2H), 3.66 (s, 3H), 1.26 (t, 3H).
Step 2

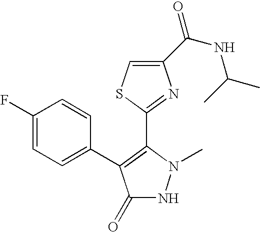

### 2-|4-(4-fluoro-phenyl)-2-methyl-5-oxo-2,5-dihydro-1H-pyrazol-3-yl|-thiazole-4-carboxylic acid isopropyl amide

A 10 mL sealed tube was charged with 2-[4-(4-fluoro-phenyl)-2-methyl-5-oxo-2,5-dihydro-1H-pyrazol-3-yl]-thiazole-4-carboxylic acid ethyl (60 mg, 0.16 mmol) and isopropylamine (3 mL). The resulting solution was stirred at 65° C. for 20 hours. The reaction was monitored by TLC (CH₂Cl₂/MeOH=15:1). Work-up: the mixture was concentrated in vacuo to afford a residue that was purified by column chromatography eluted with 200:1 to 100:1 CH₂Cl₂/MeOH gradient solvent system to give 23 mg (39%) of title compound as a yellow solid. ¹H NMR (400 MHz, DMSO) δ: 10.95 (s, 1H), 8.07 (s, 1H), 7.49 (t, 2H), 7.19 (t, 2H), 6.81 (d, 1H), 3.91 (q, 1H), 3.66 (s, 3H), 1.06 (d, 6H); MS: 360 [M+H]⁺.

### EXAMPLE 2183

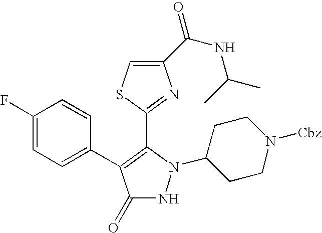

### 4-|4-(4-Fluoro-phenyl)-5-(4-isopropylcarbamoyl-thiazol-2-yl)-3-oxo-2,3-dihydro-pyrazol-1-yl|-piperidine-1-carboxylic acid benzyl ester

Step 1

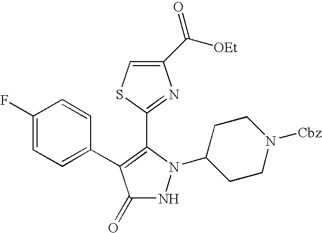

### 4-|5-(4-Ethoxycarbonyl-thiazol-2-yl)-4-(4-fluoro-phenyl)-3-oxo-2,3-dihydro-pyrazol-1-yl|-piperidine-1-carboxylic acid benzyl ester

A 50 mL round bottom flask was charged with 2-[2-Ethoxycarbonyl-2-(4-fluoro-phenyl)-acetyl]-thiazole-4-carboxylic acid ethyl ester (320 mg, 0.88 mmol, described in step 4 of Example 2180, and acetic acid (20 mL). To this solution was added 4-(N′-tert-butoxycarbonyl-hydrazino)-piperidine-1-carboxylic acid benzyl ester (920 mg, 2.64 mmol, prepared as described in Tetrahedron Letters 2005, 46(46), 7993-7996, J. Deng et. al). The resulting solution was stirred in a 120° C. in a bath of oil overnight. Reaction progress was monitored by TLC (CH₂Cl₂/MeOH=15:1). Work-up: the mixture was concentrated in vacuo to afford a residue that was dissolved in 20 mL of H₂O, extracted with CHCl₃ (3×50 mL), washed with 50 mL of saturated solution of NaHCO₃, dried over Na₂SO₄, then purified by column chromatography eluted with 500:1 to 150:1 CH₂Cl₂/MeOH. This resulted in 200 mg (38%) of title compound as a yellow oil. MS: 551 [M+H]⁺.
Step 2

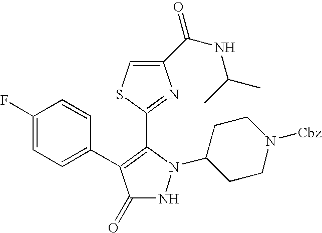

### 4-|4-(4-Fluoro-phenyl)-5-(4-isopropylcarbamoyl-thiazol-2-yl)-3-oxo-2,3-dihydro-pyrazol-1-yl|-piperidine-1-carboxylic acid benzyl ester

A 10 mL sealed tube was charged with a solution of 4-[5-(4-ethoxycarbonyl-thiazol-2-yl)-4-(4-fluoro-phenyl)-3-oxo-2,3-dihydro-pyrazol-1-yl]-piperidine-1-carboxylic acid benzyl ester (200 mg, 0.33 mmol) and isoproyl amine (6 mL). The resulting solution was allowed to stir at 65° C. for 48 hours. The mixture was then cooled to room temperature and concentrated in vacuo to afford a residue that was purified by silica gel column chromatography eluted with 150:1 CH₂Cl₂/MeOH. This gave 70 mg (37%) of the title compound as white solid. ¹HNMR (400 MHz, CDCl₃) δ: 8.58 (m, 1H), 7.76 (s, 1H), 7.37 (s, 7H), 7.00 (t, 2H), 6.72 (d, 1H), 5.09 (d, 2H), 4.46 (s, 1H), 4.32 (m, 2H), 3.94 (d, 1H), 2.97 (m, 2H), 2.19 (q, 2H), 2.01 (q, 2H), 1.09 (d, 6H); MS: 562[M−H]⁺.

### EXAMPLE 2184

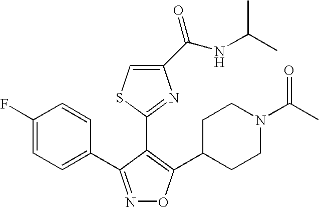

### 2-|5-(1-Acetyl-piperidin-4-yl)-3-(4-fluoro-phenyl)-isoxazol-4-yl|-thiazole-4-carboxylic acid isopropylamide

A 8 mL vial with a pearcable cap was charged with 2-[3-(4-fluoro-phenyl)-5-piperidin-4-yl-isoxazol-4-yl]-thiazole-4-carboxylic acid isopropylamide (80 mg, 0.19 mmol) from step 7 of example 13 and dissolved in DCM (1.5 mL). To this solution was added TEA (0.195 mg, 1.9 mmol) followed by acetic anhydride (29 mg, 0.28 mmol) at room temperature. This mixture was allowed to stir at this temperature for 2 h. The conversion was monitored by TLC and/or LCMS. The reaction mixture %% as then concentrated down to dryness, redissolved in a 1:1 mixture of MeOH/DMSO (1 mL) and purified by RP C18 column eluted with 20-60% MeCN in water in the presence of 0.1% TFA to afford the product as white solid (28 mg). ¹H NMR (400 MHz, CD₃OD) δ: 8.21 (s, 1H), 7.57-7.54 (m, 2H), 7.25-7.21 (m, 2H), 4.65-4.62 (m, 1H), 4.21-4.15 (m, 1H), 4.09-4.06 (m, 1H), 3.74-3.68 (m, 1H), 3.30-3.26 (m, 1H), 2.82 (t, 1H), 2.15 (s, 3H), 2.01-2.04 (m, 1H), 2.00-1.79 (m, 2H) 1.25 (d, 6H); LCMS (M+1)⁺: 457.85.

### EXAMPLE 2185

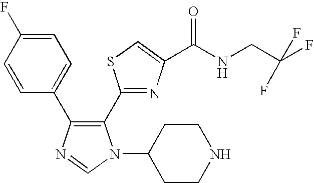

### 2-|5-(4-Fluoro-phenyl)-3-piperidin-4-yl-3H-imidazol-4-yl|-thiazole-4-carboxylic acid (2,2,2-trifluoro-ethyl)-amide

Step 1

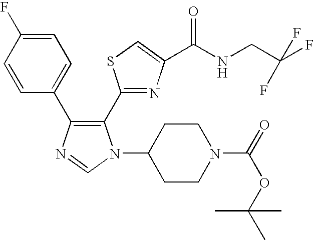

### 4-{4-(4-Fluoro-phenyl)-5-|4-(2,2,2-trifluoro-ethylcarbamoyl)-thiazol-2-yl|-imidazol-1-yl}-piperidine-1-carboxylic acid tert-butyl ester

To a stirred solution of 4-[5-(4-carboxy-thiazol-2-yl)-4-(4-fluoro-phenyl)-imidazol-1-yl]-piperidine-1-carboxylic acid tert-butyl ester (65 mg, 0.14 mmol, prepared as described in step 2 of example 95) in DCM (2 mL) at room temperature was added 2,2,2-trifluoroethylamine, 1-ethyl-3-[3-(dimethylamino)propyl]-carbodiimide hydrochloride (EDC, 32 mg, 0.17 mmol), and DIEA (61 μL, 0.35 mmol). After 1 hour, the mixture was poured on to a silica gel column, eluting with 70% EtOAc in hexanes to afford the title compound as a colorless solid. LCMS: 554.4 (M+1)⁺.
Step 2

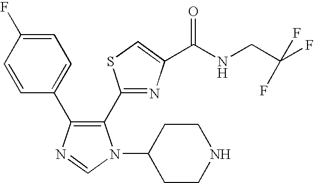

### 2-|5-(4-Fluoro-phenyl)-3-piperidin-4-yl-3H-imidazol-4-yl|-thiazole-4-carboxylic acid (2,2,2-trifluoro-ethyl)-amide

To a stirred solution of 4-{4-(4-fluoro-phenyl)-5-[4-(2,2,2-trifluoro-ethylcarbamoyl)-thiazol-2-yl]-imidazol-1-yl}-piperidine-1-carboxylic acid tert-butyl ester (6 mg, 0.01 mmol) in DCM (200 μL) at room temperature was added 20% TFA in DCM (1 mL). After 30 min, the reaction was diluted with toluene (10 mL) and concentrated to dryness in vacuo to afford the title compound as a colorless solid. ¹H NMR (400 MHz, CD₃OD) δ 8.79 (s, 1H), 8.42 (s, 1H), 7.51 (m, 2H), 7.22 (m, 2H), 7.15 (m, 1H), 5.12 (m, 1H), 4.15 (m, 2H), 3.60 (m, 2H), 3.17 (t, 2H), 2.52 (d, 2H), 2.30 (m, 2H). LCMS: 454.4 (M+1)⁺.

### EXAMPLE 2186

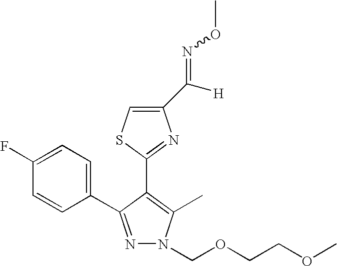

### 2-|3-(4-Fluoro-phenyl)-1-(2-methoxy-ethoxymethyl)-5-methyl-1H-pyrazol-4-yl|-thiazole-4-carbaldehyde O-methyl-oxime

To a stirred solution of 2-[3-(4-Fluoro-phenyl)-1-(2-methoxy-ethoxymethyl)-5-methyl-1H-pyrazol-4-yl]-thiazole-4-carbaldehyde (120 mg, 0.32 mmol, described in Example 2161 in MeOH (0.5 mL) at room temperature was added O-methyl-hydroxylamine hydrochloride (54 mg, 0.64 mmol), sodium sulfate (91 mg, 0.64 mmol) and pyridine (100 μL). After 18 hours, TLC analysis revealed disappearance of starting material. LC/MS analysis confirmed the presence of 2 separable oxime isomers. The mixture was purified via C18 reverse-phase preparatory HPLC (15 min gradient of 30% to 60% ACN in H2O mobile phase with 0.1% TFA), collecting 2-[3-(4-Fluoro-phenyl)-1-(2-methoxy-ethoxymethyl)-5-methyl-1H-pyrazol-4-yl]-thiazole-4-carbaldehyde O-methyl-oxime as the first-eluting peak off of the column. ¹H NMR (400 MHz, CD₃OD) δ 8.12 (s, 1H), 7.59 (s, 1H), 7.51 (m, 2H), 7.24 (m, 2H), 5.27 (s, 2H), 3.91 (s, 3H), 3.65 (m, 2H), 3.47 (m, 2H), 3.29 (s, 3H), 2.52 (s, 3H). LCMS: 405.2 (M+1)⁺.

### EXAMPLE 2187

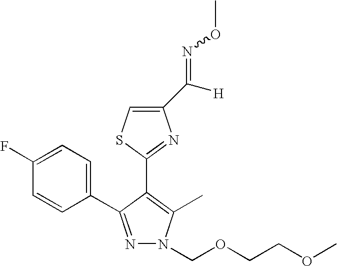

### 2-|3-(4-Fluoro-phenyl)-1-(2-methoxy-ethoxymethyl)-5-methyl-1H-pyrazol-4-yl|-thiazole-4-carbaldehyde O-methyl-oxime

To a stirred solution of 2-[3-(4-fluoro-phenyl)-1-(2-methoxy-ethoxymethyl)-5-methyl-1H-pyrazol-4-yl]-thiazole-4-carbaldehyde (120 mg, 0.32 mmol, described in Example 2161 in MeOH (0.5 mL) at room temperature was added O-methyl-hydroxylamine hydrochloride (54 mg, 0.64 mmol), sodium sulfate (91 mg, 0.64 mmol) and pyridine (100 μL). After 18 hours, TLC analysis revealed disappearance of starting material. LC/MS analysis confirmed the presence of 2 separable oxime isomers. The mixture was purified via C18 reverse-phase preparatory HPLC (15 min gradient of 30% to 60% ACN in H2O mobile phase with 0.1% TFA), collecting 2-[3-(4-Fluoro-phenyl)-1-(2-methoxy-ethoxymethyl)-5-methyl-1H-pyrazol-4-yl]-thiazole-4-carbaldehyde O-methyl-oxime as the second-eluting peak off of the column. ¹H NMR (400 MHz, CD₃OD) δ 8.20 (s, 1H), 7.61 (s, 1H), 7.51 (m, 2H), 7.23 (m, 2H), 5.27 (s, 2H), 4.03 (s, 3H), 3.67 (m, 2H), 3.47 (m, 2H), 3.28 (s, 3H), 2.52 (s, 3H). LCMS: 405.2 (M+1)⁺.

### EXAMPLE 2188

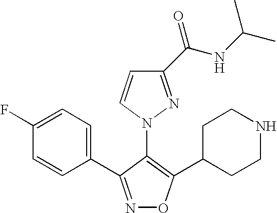

### 1-|3-(4-Fluoro-phenyl)-5-piperidin-4-yl-isoxazol-4-yl|-1H-pyrazole-3-carboxylic acid isopropylamide

Step 1

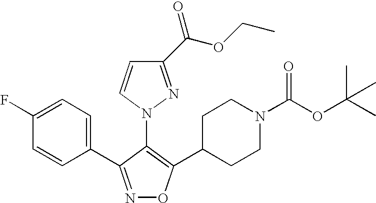

### 4-|4-(3-Ethoxycarbonyl-pyrazol-1-yl)-3-(4-fluoro-phenyl)-isoxazol-5-yl|-piperidine-1-carboxylic acid tert-butyl ester

A nitrogen-flushed 100 mL round bottom flask was charged with ethyl-pyrazole-3-carboxylate (420 mg, 3.0 mmol, prepared as described in J. Am. Chem. Soc. 2000, 122, 10810), 4-[3-(4-fluoro-phenyl)-4-iodo-isoxazol-5-yl]-piperidine-1-carboxylic acid tert-butyl ester (1.7 g, 3.6 mmol, prepared as described in Step 3 of Example 35), CuI (28 mg, 0.15 mmol), (1R,2R)-diaminomethylcyclohexane (85 mg, 0.6 mmol), and potassium carbonate (869 mg, 6.3 mmol). The solid mixture was evacuated and back-filled with nitrogen 3 times, then dry toluene (2 mL) was added via syringe. The resulting slurry was capped with a reflux condenser and heated for 3 days in a 110° C. oil bath. The crude mixture was then rinsed in to a separatory funnel containing EtOAc (200 mL) and water (50 mL). The organic layer was washed with an additional portion of water (50 mL), then dried over MgSO4 and concentrated. The crude residue was purified by reverse-phase preparative HPLC to afford the title compound (140 mg) as an off-white solid. LCMS: 485.4 (M+1)⁺.
Step 2

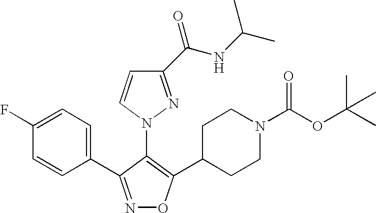

### 4-|3-(4-Fluoro-phenyl)-4-(3-isopropylcarbamoyl-pyrazol-1-yl)-isoxazol-5-yl|-piperidine-1-carboxylic acid tert-butyl ester

The title compound was prepared analogously to 4-[3-(4-fluoro-phenyl)-4-(4-isopropylcarbamoyl-thiazol-2-yl)-isoxazol-5-yl]-piperidine-1-carboxylic acid tert-butyl ester (Step 6 of Example 35) by substituting 4-[4-(3-ethoxycarbonyl-pyrazol-1-yl)-3-(4-fluoro-phenyl)-isoxazol-5-yl]-piperidine-1-carboxylic acid tert-butyl ester for 4-[4-(4-ethoxycarbonyl-thiazol-2-yl)-3-(4-fluoro-phenyl)-isoxazol-5-yl]-piperidine-1-carboxylic acid tert-butyl ester in that step. LCMS: 498.5 (M+1)⁺.
Step 3

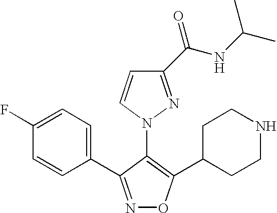

### 1-|3-(4-Fluoro-phenyl)-5-piperidin-4-yl-isoxazol-4-yl|-1H-pyrazole-3-carboxylic acid isopropylamide

To a stirred solution of 4-[3-(4-fluoro-phenyl)-4-(3-isopropylcarbamoyl-pyrazol-1-yl)-isoxazol-5-yl]-piperidine-1-carboxylic acid tert-butyl ester (120 mg, 0.24 mmol) in DCM (1 mL) was added 25% TFA in DCM (2 mL). The reaction was stirred for 1 hr, then diluted with toluene (30 ml) and concentrated to dryness in vacuo. The crude residue was purified by reverse-phase preparative HPLC to afford the title compound as a white powder. ¹H NMR (400 MHz, CD₃OD) δ 8.28 (d, 1H), 8.08 (m, 2H), 7.79 (m, 1H), 7.28 (m, 2H), 6.97 (d, 1H), 4.23 (m, 1H), 3.95 (m, 1H), 3.53 (d, 2H), 3.22 (m, 2H), 2.20 (m, 4H), 1.28 (d, 6H). LCMS: 405.2 (M+1)⁺.

### EXAMPLE 2189

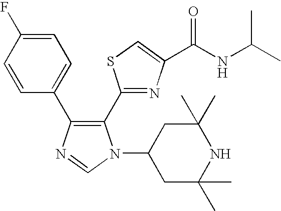

### 2-|5-(4-Fluoro-phenyl)-3-(2,2,6,6-tetramethyl-piperidin-4-yl)-3H-imidazol-4-yl|-thiazole-4-carboxylic acid isopropylamide

Step 1

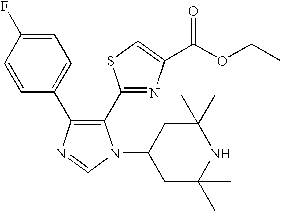

### 2-|5-(4-Fluoro-phenyl)-3-(2,2,6,6-tetramethyl-piperidin-4-yl)-3H-imidazol-4-yl|-thiazole-4-carboxylic acid ethyl ester

The title compound was prepared analogously to 2-[5-(4-fluoro-phenyl)-3-methyl-3H-imidazol-4-yl]-thiazole-4-carboxylic acid ethyl ester (Step 1, Example 86), where 4-amino-2,2,6,6-tetramethylpiperidine was substituted for methylamine in that step. LCMS: 457.6 (M+1)⁺.
Step 2

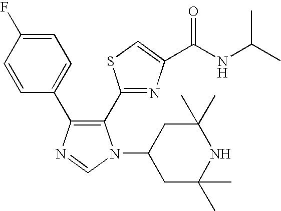

### 2-|5-(4-Fluoro-phenyl)-3-(2,2,6,6-tetra methyl-piperidin-4-yl)-3H-imidazol-4-yl|-thiazole-4-carboxylic acid isopropylamide

The title compound was prepared analogously to 2-[5-(4-fluoro-phenyl)-3-methyl-3H-imidazol-4-yl]-thiazole-4-carboxylic acid isopropylamide in step 2 of example 86 by substituting 2-[5-(4-fluoro-phenyl)-3-methyl-3H-imidazol-4-yl]-thiazole-4-carboxylic acid ethyl ester with 2-[5-(4-fluoro-phenyl)-3-(2,2,6,6-tetramethyl-piperidin-4-yl)-3H-imidazol-4-yl]-thiazole-4-carboxylic acid ethyl ester. ¹H NMR (400 MHz, CD₃OD) δ 8.43 (m, 1H), 8.34 (s, 1H), 8.28 (s, 1H), 7.46 (m, 2H), 7.17 (m, 1H), 5.48 (m, 1H), 4.24 (m, 1H), 2.33 (m, 2H), 2.07 (m, 2H), 1.50 (s, 12H), 1.25 (d, 6H). LCMS: 470.7 (M+1)⁺.

### EXAMPLE 2190

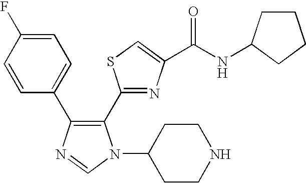

### 2-|5-(4-Fluoro-phenyl)-3-piperidin-4-yl-3H-imidazol-4-yl|-thiazole-4-carboxylic acid cyclopentylamide

Step 1

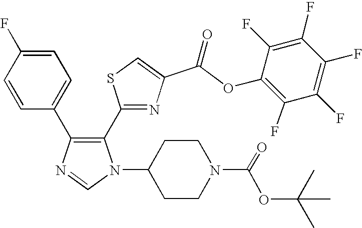

### 4-|4-(4-Fluoro-phenyl)-5-(4-pentafluorophenyloxycarbonyl-thiazol-2-yl)-imidazol-1-yl|-piperidine-1-carboxylic acid tert-butyl ester

To a solution of 4-[5-(4-carboxy-thiazol-2-yl)-4-(4-fluoro-phenyl)-imidazol-1-yl]-piperidine-1-carboxylic acid tert-butyl ester (188 mg, 0.4 mmol, prepared as described in step 2 of example 95) in DMF (5 mL) at room temperature was added pyridine (35 μL, 0.44 mmol), followed by pentafluorophenyl trifluoroacetate (82 μL, 0.48 mmol). The mixture was stirred for 10 min, at which time LCMS analysis revealed full conversion to title compound. Following aqueous extraction, the product 4-[4-(4-fluoro-phenyl)-5-(4-pentafluorophenyloxycarbonyl-thiazol-2-yl)-imidazol-1-yl]-piperidine-1-carboxylic acid tert-butyl ester determined to be of sufficient purity to carry on to the next step. LCMS: 639.7 (M+1)⁺.
Step 2

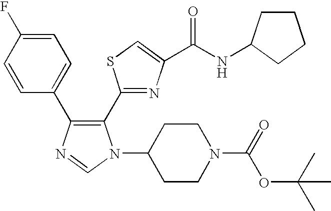

### 4-|5-(4-Cyclopentylcarbamoyl-thiazol-2-yl)-4-(4-fluoro-phenyl)-imidazol-1-yl|-piperidine-1-carboxylic acid tert-butyl ester

To a stirred solution of 4-[4-(4-fluoro-phenyl)-5-(4-pentafluorophenyloxycarbonyl-thiazol-2-yl)-imidazol-1-yl]-piperidine-1-carboxylic acid tert-butyl ester (127 mg, 0.2 mmol) in DMF (2 mL) at room temperature was added cyclopentylamine (100 μL, 1.0 mmol). After 20 min, the reaction was determined to be complete by LCMS analysis. The mixture was rinsed in to a separatory funnel containing 1:1 hexanes:EtOAc (50 mL). The resulting solution was washed with HCl (30 mL, 1N aqueous), NaOH (30 mL, 1N aqueous), and brine (50 mL), then dried, filtered, and concentrated to dryness in vacuo to afford the title compound as a tan solid that was determined to be sufficiently by LCMS to carry on to the next step. LCMS: 540.8 (M+1)⁺.
Step 3

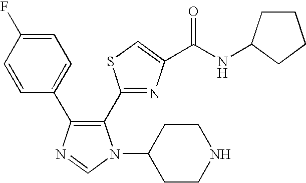

### 2-|5-(4-Fluoro-phenyl)-3-piperidin-4-yl-3H-imidazol-4-yl|-thiazole-4-carboxylic acid cyclopentylamide

To a stirred mixture of 4-[5-(4-cyclopentylcarbamoyl-thiazol-2-yl)-4-(4-fluoro-phenyl)-imidazol-1-yl]-piperidine-1-carboxylic acid tert-butyl ester (102 mg, 0.19 mmol) in DCM (8 mL) was added trifluoroacetic acid (2 mL). After 30 min. full conversion to the title compound was observed by LCMS. The mixture was diluted with toluene (30 mL), concentrated to dryness in vacuo, and purified by reverse phase preparatory HPLC, to afford the title compound as a white solid. ¹H NMR (400 MHz, CD₃OD) δ 8.39 (s, 1H), 8.24 (s, 1H), 7.46 (m, 2H), 7.17 (m, 2H), 4.98 (m, 1H), 4.35 (m, 1H), 3.59 (m, 2H), 3.18 (m, 2H), 2.50 (m, 2H), 2.23 (m, 2H), 2.06 (m, 2H), 1.77 (m, 2H), 1.56-1.85 (m, 4H) LCMS: 440.8 (M+1)⁺.

### EXAMPLE 2191

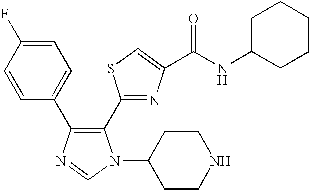

### 2-|5-(4-Fluoro-phenyl)-3-piperidin-4-yl-3H-imidazol-4-yl|-thiazole-4-carboxylic acid cyclohexylamide

The title compound was prepared analogously to 2-[5-(4-fluoro-phenyl)-3-piperidin-4-yl-3H-imidazol-4-yl]-thiazole-4-carboxylic acid cyclopentylamide Example 2190, where cyclohexylamine was substituted for cyclopentylamine in step 2 of that sequence. ¹H NMR (400 MHz, CD₃OD) δ 8.53 (s, 1H), 8.29 (s, 1H), 7.47 (m, 2H), 7.19 (m, 2H), 5.02 (m, 1H), 3.89 (m, 1H), 3.60 (d, 2H), 3.17 (t, 2H), 2.52 (d, 2H), 2.25 (m, 2H), 1.97 (m, 2H), 1.80 (m, 2H), 1.69 (m, 1H), 1.35-1.48 (m, 4H), 1.26 (m, 1H). LCMS: 454.8 (M+1)⁺.

### EXAMPLE 2192

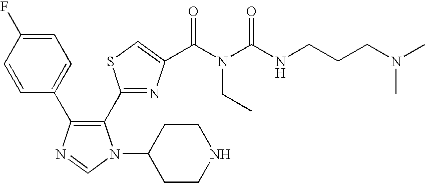

### 3-(3-Dimethylamino-propyl)-1-ethyl-1-{2-|5-(4-fluoro-phenyl)-3-piperidin-4-yl-3H-imidazol-4-yl|-thiazole-4-carbonyl}-urea

Step 1

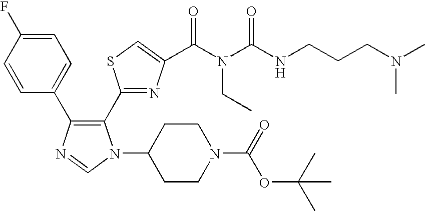

To a solution of 4-[5-(4-carboxy-thiazol-2-yl)-4-(4-fluoro-phenyl)-imidazol-1-yl]-piperidine-1-carboxylic acid tert-butyl ester (94 mg, 0.2 mmol, prepared as described in step 2 of example 95) in DCM (20 mL) at room temperature was added 1-ethyl-3-[3-(dimethylamino)propyl]-carbodiimide hydrochloride (EDC, 46 mg, 0.24 mmol), DIEA (105 μL, 0.6 mmol), and cyclohexylamine (34 μL, 0.3 mmol). After 18 hours, the reaction was poured in to a separatory funnel containing DCM (50 mL) and washed with HCl (30 mL, 1N aqueous), NaHCO₃ (30 mL, sat. aqueous), and brine (30 mL). The organic layer was dried over MgSO₄, filtered, and concentrated to dryness in vacuo to afford the title compound as a tan solid that was carried on to the next step without further purification. LCMS: 628.6 (M+1)⁺.
Step 2

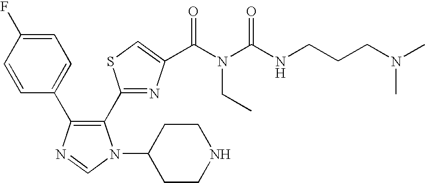

### 3-(3-Dimethylamino-propyl)-1-ethyl-1-{2-|5-(4-fluoro-phenyl)-3-piperidin-4-yl-3H-imidazol-4-yl|-thiazole-4-carbonyl}-urea

To a solution of 4-[5-{4-[3-(3-dimethylamino-propyl)-1-ethyl-ureidocarbonyl]-thiazol-2-yl}-4-(4-fluoro-phenyl)-imidazol-1-yl]-piperidine-1-carboxylic acid tert-butyl ester (60 mg, 0.1 mmol) in DCM (1 mL) at room temperature was added trifluoroacetic acid (250 μL). After 1 hr, LCMS analysis revealed full conversion to the title compound. The mixture was diluted with toluene (20 mL), concentrated to dryness in vacuo, and purified via reverse phase preparatory HPLC to afford the title compound as a white solid. LCMS: 528.5 (M+1)⁺.

### EXAMPLE 2193

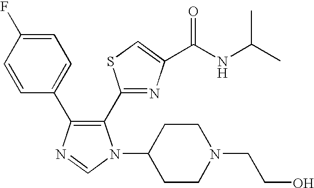

### 2-{5-(4-Fluoro-phenyl)-3-|1-(2-hydroxy-ethyl)-piperidin-4-yl|-3H-imidazol-4-yl}-thiazole-4-carboxylic acid isopropylamide

To a stirred solution of 2-[5-(4-fluoro-phenyl)-3-piperidin-4-yl-3H-imidazol-4-yl]-thiazole-4-carboxylic acid isopropylamide, (30 mg, 0.06 mmol, prepared as described in example 91) in DMF at room temperature was added diisopropylethylamine (50 μL, 0.29 mmol), followed by 2-bromoethanol (15 μL, 0.22 mmol). The mixture was warmed to 50° C. and left to stir for 2.5 hr. at which time LCMS analysis revealed full conversion to title compound. The mixture was then purified by direct injection on to a reverse phase preparatory HPLC, to afford the title compound as a white solid. ¹H NMR (400 MHz, CD₃OD) δ 8.56 (s, 1H), 8.31 (s, 1H), 7.47 (m, 2H), 7.18 (m, 2H), 4.23 (m, 1H), 3.90 (t, 2H), 3.81 (m, 2H), 3.60 (m, 1H), 3.15-3.35 (m, 4H), 2.56 (m, 2H), 2.42 (m, 2H), 1.28 (d, 6H). LCMS: 458.4 (M+1)⁺.

### EXAMPLE 2194

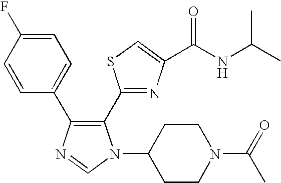

### 2-|3-(1-Acetyl-piperidin-4-yl)-5-(4-fluoro-phenyl)-3H-imidazol-4-yl|-thiazole-4-carboxylic acid isopropylamide

To a stirred solution of 2-[5-(4-fluoro-phenyl)-3-piperidin-4-yl-3H-imidazol-4-yl]-thiazole-4-carboxylic acid isopropylamide, (52 mg, 0.10 mmol, prepared as described in example 91) in dry DCM (2 mL) at room temperature was added diisopropylethylamine (86 μL, 0.5 mmol), followed by acetyl chloride (8 μL, 0.12 mmol). After stirring for 5 minutes, LCMS analysis revealed full conversion to title compound. The reaction was quenched via the addition of EtOH (1 mL), then concentrated to dryness in vacuo. The crude residue was then purified by reverse phase preparatory HPLC, to afford the title compound as a white solid. ¹H NMR (400 MHz, CDCl₃) δ 9.19 (s, 1H), 8.38 (s, 1H), 7.43 (m, 2H), 7.15 (m, 2H), 7.04 (d, 1H), 4.89 (m, 1H), 4.70 (m, 1H), 4.28 (m, 1H), 4.08 (m, 1H), 3.18 (m, 1H), 2.61 (m, 1H), 2.20-2.40 (m, 3H), 2.19 (s, 3H), 2.01 (m, 1H), 1.30 (d, 6H). LCMS: 456.1 (M+1)⁺.

### EXAMPLE 2195

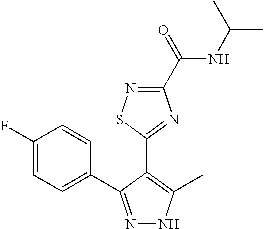

### 5-|3-(4-Fluoro-phenyl)-5-methyl-1H-pyrazol-4-yl|-|1,2,4|thiadiazole-3-carboxylic acid isopropylamide

Step 1

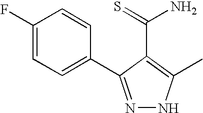

### 3-(4-Fluoro-phenyl)-5-methyl-1H-pyrazole-4-carbothioic acid amide

A 500 ml roundbottom flask was charged with 3-(4-Fluoro-phenyl)-1-(2-methoxy-ethoxymethyl)-5-methyl-1H-pyrazole-4-carboxylic acid amide (32.2 g, 105 mmol, described in step 6 of example 79), 1,2-dimethoxyethane (400 ml) and phosphorus pentasulfide (23.3 g) in several batches, while maintaining the contents at room temperature. The resulting solution was stirred for 3 hours at 30° C. Reaction progress was monitored by TLC (CH₂Cl₂/MeOH=15:1). Work-up: the mixture was concentrated and purified by column chromatography with a 100:1 CH₂Cl₂/MeOH, yielding 5.7 g of 3-(4-fluorophenyl)-5-methyl-1H-pyrazole-4-carbothioamide as a white solid, and 15.7 g of 3-(4-fluorophenyl)-1-((2-methoxyethoxy)methyl)-5-methyl-1H-pyrazole-4-carbothioamide as a yellow solid.
Step 2

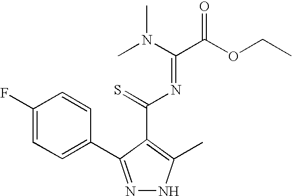

### Dimethylamino-|3-(4-fluoro-phenyl)-5-methyl-1H-pyrazole-4-carbothioylimino|-acetic acid ethyl ester

A 500 ml round bottom flask was charged with 3-(4-fluorophenyl)-5-methyl-1H-pyrazole-4-carbothioamide (600 mg, 2.55 mmol), ethyl 2-(dimethylamino)-2,2-diethoxyacetate (2 g, 9.12 mmol), and THF (20 ml). The resulting solution stirred for 26 hours at 60 degrees C. The reaction progress was monitored by TLC (CH₂Cl₂/MeOH=15:1). The mixture was concentrated by evaporation under vacuum using a rotary evaporator. The residue was purified by eluting through a column with a 1:3 EtOAc/PE solvent system. This resulted in 0.58 g (45%) of ethyl 2-(dimethylamino)-2-(3-(4-fluorophenyl)-5-methyl-1H-pyrazole-4-carbothioamido)acetate as orange oil.
Step 3

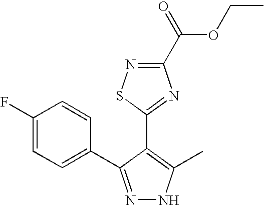

### 5-|3-(4-Fluoro-phenyl)-5-methyl-1H-pyrazol-4-yl|-|1,2,4|thiadiazole-3-carboxylic acid ethyl ester

A 100 ml round bottom flask was purged with nitrogen, then charged with (E)-ethyl 2-(dimethylamino)-2-(3-(4-fluorophenyl)-5-methyl-1H-pyrazole-4-carbothioamido)acetate (780 mg, 2.15 mmol), absolute ethanol (20 ml), pyridine (850 mg, 10.75 mmol), and hydroxylamine-O-sulfonic acid (500 mg, 4.42 mmol) in methanol (20 ml). The resulting solution was stirred for 2 hours at room temperature. Reaction progress was monitored by TLC (EtOAc/PE=1:1). Work-up: the mixture was concentrated, dissolved in 150 ml of AcOEt, washed with K₂CO₃ (aq.), brine, dried over Na₂SO₄, concentrated, and purified by column chromatography (1:30 EtOAc/PE solvent system). This gave 350 mg (50%) of the title compound mixed with the analogous oxadiazole. These compounds were purified by RPHPLC, and then recrystallized from EtOAc/PE=1:1.
Step 4

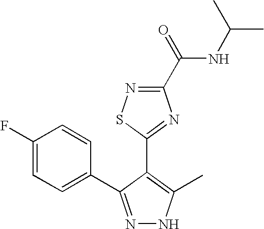

### 5-|3-(4-Fluoro-phenyl)-5-methyl-1H-pyrazol-4-yl|-|1,2,4|thiadiazole-3-carboxylic acid isopropylamide

To a stirred solution of 5-[3-(4-Fluoro-phenyl)-5-methyl-1H-pyrazol-4-yl]-[1,2,4]thiadiazole-3-carboxylic acid ethyl ester (120 mg, 0.36 mmol) in toluene (5 mL), was added MeAlCl(NH-iPr) (1.1 mL of a 0.67 M solution in toluene, 0.72 mmol, prepared as described in Synth. Comm. 12, 13, 989.) dropwise via syringe. The resulting mixture was warmed to 80° C. and left to stir for 2 hrs, then cooled to room temperature and poured on to a vigorously stirred slurry of sodium sulfate decahydrate (25 g) in DCM (100 mL). After 1 hr. the mixture was filtered, and the resulting filtrate was dried over MgSO₄, filtered, and concentrated in vacuo to afford the title compound (99 mg, 80% yield) as a white solid. ¹H NMR (400 MHz, CDCl₃) δ 7.59 (m, 2H), 7.13 (m, 2H), 6.73 (d, 1H), 4.28 (m, 1H), 2.65 (s, 3H), 1.26 (d, 6H). LCMS: 346.2 (M+1)⁺.

### EXAMPLE 2196

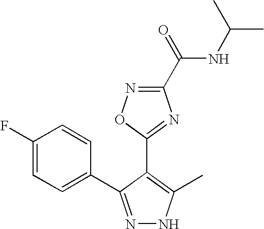

### 5-|3-(4-Fluoro-phenyl)-5-methyl-1H-pyrazol-4-yl|-|1,2,4|oxadiazole-3-carboxylic acid isopropylamide

To a stirred solution of 5-[3-(4-Fluoro-phenyl)-5-methyl-1H-pyrazol-4-yl]-[1,2,4]oxadiazole-3-carboxylic acid ethyl ester (68 mg, 0.22 mmol, described in step 3 of Example 2196) in toluene (3 mL), was added dropwise MeAlCl(NH-iPr) (800 μL of a 0.67 M solution in toluene, 0.55 mmol, prepared as described in Synth. Comm. 12, 13, 989). The resulting mixture was warmed to 80° C. and left to stir for 2 hrs, then cooled to room temperature and poured on to a vigorously stirred slurry of sodium sulfate decahydrate (25 g) in DCM (100 mL). After 1 hr, the mixture was filtered, and the resulting filtrate was dried over MgSO₄, filtered, and concentrated in vacuo to afford the title compound (57 mg, 80% yield), that was determined to be sufficiently pure by available analytical methods. ¹H NMR (400 MHz, CDCl₃) δ 7.56 (m, 2H), 7.08 (m, 2H), 6.75 (d, 1H), 4.26 (m, 1H), 2.54 (s, 3H), 1.25 (d, 6H). LCMS: 330.7 (M+1)⁺.

The following Compounds are represented herein using the Simplified Molecular Input Line Entry System, or SMILES. SMILES is a modern chemical notation system, developed by David Weininger and Daylight Chemical Information Systems, Inc., that is built into all major commercial chemical structure drawing software packages. Software is not needed to interpret SMILES text strings, and an explanation of how to translate SMILES into structures can be found in Weininger, D., J. Chem. Inf. Comput. Sci. 1988, 28, 31-36.

### Examples 98-2157 Prepared by Parallel Synthesis

### EXAMPLES 98-334

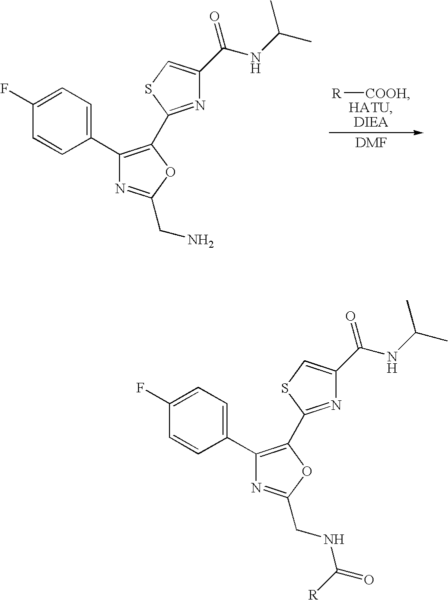

Starting amino methyl oxazole was prepared as described in Example 43, but omitting the Boc protection step. Where R—COOH is a carboxylic acid selected to afford Examples 98-334, which were prepared by General Procedure 1.

| | |
|---|---|
| Example 98 | FC═3C═CC(C1═C(OC(═N1)CN)C2═NC(═CS2)C(NC(C)C)═O)═CC═3 |
| Example 99 | O═C(C═O)NCC2═NC(═C(C1═NC(C(═O)NC(C)C)═CS1)O2)C3═CC═C(F)C═C3 |
| Example 100 | O═C(CC#N)NCC2═NC(═C(C1═NC(C(═O)NC(C)C)═CS1)O2)C3═CC═C(F)C═C3 |
| Example 101 | OC(C)C(═O)NCC2═NC(═C(C1═NC(C(═O)NC(C)C)═CS1)O2)C3═CC═C(F)C═C3 |
| Example 102 | O═C(CC)C(═O)NCC2═NC(═C(C1═NC(C(═O)NC(C)C)═CS1)O2)C3═CC═C(F)C═C3 |
| Example 103 | O═C(C(C)(C)C)NCC2═NC(═C(C1═NC(C(═O)NC(C)C)═CS1)O2)C3═CC═C(F)C═C3 |
| Example 104 | O═C(CC(C)C)NCC2═NC(═C(C1═NC(C(═O)NC(C)C)═CS1)O2)C3═CC═C(F)C═C3 |
| Example 105 | [H]N4C(C(═O)NCC2═NC(═C(C1═NC(C(═O)NC(C)C)═CS1)O2)C3═CC═C(F)C═C3)═CN═C4 |
| Example 106 | [H]N1C(═NC═C1)C(═O)NCC3═NC(═C(C2═NC(C(═O)NC(C)C)═CS2)O3)C4═CC═C(F)C═C4 |
| Example 107 | C1CC(═CC1)C(═O)NCC3═NC(═C(C2═NC(C(═O)NC(C)C)═CS2)O3)C4═CC═C(F)C═C4 |
| Example 108 | FC(F)(C(═O)NCC2═NC(═C(C1═NC(C(═O)NC(C)C)═CS1)O2)C3═CC═C(F)C═C3)F |
| Example 109 | C4CCOC4C(═O)NCC2═NC(═C(C1═NC(C(═O)NC(C)C)═CS1)O2)C3═CC═C(F)C═C3 |
| Example 110 | O═C(C)NCC(═O)NCC2═NC(═C(C1═NC(C(═O)NC(C)C)═CS1)O2)C3═CC═C(F)C═C3 |
| Example 111 | C═4C═CC═CC═4C(═O)NCC2═NC(═C(C1═NC(C(═O)NC(C)C)═CS1)O2)C3═CC═C(F)C═C3 |
| Example 112 | C═4C═CN═CC═4C(═O)NCC2═NC(═C(C1═NC(C(═O)NC(C)C)═CS1)O2)C3═CC═C(F)C═C3 |
| Example 113 | C1═CC(═CC═N1)C(═O)NCC3═NC(═C(C2═NC(C(═O)NC(C)C)═CS2)O3)C4═CC═C(F)C═C4 |
| Example 114 | C1═NC(═CC═C1)C(═O)NCC3═NC(═C(C2═NC(C(═O)NC(C)C)═CS2)O3)C4═CC═C(F)C═C4 |
| Example 115 | OC(CCl)C(═O)NCC2═NC(═C(C1═NC(C(═O)NC(C)C)═CS1)O2)C3═CC═C(F)C═C3 |
| Example 116 | [H]N1N═C(C═C1C)C(═O)NCC3═NC(═C(C2═NC(C(═O)NC(C)C)═CS2)O3)C4═CC═C(F)C═C4 |
| Example 117 | O═C(C═1N═CN(C═1)C)NC C3═NC(═C(C2═NC(C(═O)NC(C)C)═CS2)O3)C4═CC═C(F)C═C4 |
| Example 118 | C1CC(CCC1)C(═O)NCC3═NC(═C(C2═NC(C(═O)NC(C)C)═CS2)O3)C4═CC═C(F)C═C4 |
| Example 119 | N4═CSC═C4C(═O)NCC2═NC(═C(C1═NC(C(═O)NC(C)C)═CS1)O2)C3═CC═C(F)C═C3 |
| Example 120 | O═C(OCC)CC(═O)NCC2═NC(═C(C1═NC(C(═O)NC(C)C)═CS1)O2)C3═CC═C(F)C═C3 |
| Example 121 | CC4═CC(C(═O)NCC2═NC(═C(C1═NC(C(═O)NC(C)C)═CS1)O2)C3═CC═C(F)C═C3)═CC═C4 |
| Example 122 | O═C(C1═CC═CC═C1C)NCC3═NC(═C(C2═NC(C(═O)NC(C)C)═CS2)O3)C4═CC═C(F)C═C4 |
| Example 123 | O═C(C1═CC═C(C)C═C1)NCC3═NC(═C(C2═NC(C(═O)NC(C)C)═CS2)O3)C4═CC═C(F)C═C4 |
| Example 124 | OC═1C═CC═C(C═1)C(═O)NCC3═NC(═C(C2═NC(C(═O)NC(C)C)═CS2)O3)C4═CC═C(F)C═C4 |
| Example 125 | OC═4C(C(═O)NCC2═NC(═C(C1═NC(C(═O)NC(C)C)═CS1)O2)C3═CC═C(F)C═C3)═CC═CC═4 |
| Example 126 | C═1C═C(C═CC═1F)C(═O)NCC3═NC(═C(C2═NC(C(═O)NC(C)C)═CS2)O3)C4═CC═C(F)C═C4 |
| Example 127 | C1═CC═C(C═C1F)C(═O)NCC3═NC(═C(C2═NC(C(═O)NC(C)C)═CS2)O3)C4═CC═C(F)C═C4 |
| Example 128 | CCCCCCCC(═O)NCC2═NC(═C(C1═NC(C(═O)NC(C)C)═CS1)O2)C3═CC═C(F)C═C3 |
| Example 129 | O═C(C1═CC═C(C#N)C═C1)NCC3═NC(═C(C2═NC(C(═O)NC(C)C)═CS2)O3)C4═CC═C(F)C═C4 |
| Example 130 | O═C(C1═CC═CC(C#N)═C1)NCC3═NC(═C(C2═NC(C(═O)NC(C)C)═CS2)O3)C4═CC═C(F)C═C4 |
| Example 131 | C═4C═CC═CC═4/C═C/C(═O)NCC2═NC(═C(C1═NC(C(═O)NC(C)C)═CS1)O2)C3═CC═C(F)C═C3 |
| Example 132 | O═C(C1═CC═CC═C1C═O)NCC3═NC(═C(C2═NC(C(═O)NC(C)C)═CS2)O3)C4═CC═C(F)C═C4 |
| Example 133 | O═C(C1═CC═C(C═O)C═C1)NCC3═NC(═C(C2═NC(C(═O)NC(C)C)═CS2)O3)C4═CC═C(F)C═C4 |
| Example 134 | CC1═CC(C)═CC═C1C(═O)NCC3═NC(═C(C2═NC(C(═O)NC(C)C)═CS2)O3)C4═CC═C(F)C═C4 |
| Example 135 | C═1C═CC═C(C═1C)CC(═O)NCC3═NC(═C(C2═NC(C(═O)NC(C)C)═CS2)O3)C4═CC═C(F)C═C4 |
| Example 136 | C═4C═CC(═C(C═4C(═O)NCC2═NC(═C(C1═NC(C(═O)NC(C)C)═CS1)O2)C3═CC═C(F)C═C3)C)C |
| Example 137 | CC1═C(C═C(C)C═C1)C(═O)NCC3═NC(═C(C2═NC(C(═O)NC(C)C)═CS2)O3)C4═CC═C(F)C═C4 |
| Example 138 | CC1═CC(═CC(C)═C1)C(═O)NCC3═NC(═C(C2═NC(C(═O)NC(C)C)═CS2)O3)C4═CC═C(F)C═C4 |
| Example 139 | CC═4C(═CC═C(C(═O)NCC2═NC(═C(C1═NC(C(═O)NC(C)C)═CS1)O2)C3═CC═C(F)C═C3)C═4)C |
| Example 140 | C═1C═C(C═CC═1CC)C(═O)NCC3═NC(═C(C2═NC(C(═O)NC(C)C)═CS2)O3)C4═CC═C(F)C═C4 |
| Example 141 | COC4═CC═C(C(═O)NCC2═NC(═C(C1═NC(C(═O)NC(C)C)═CS1)O2)C3═CC═C(F)C═C3)C═C4 |
| Example 142 | CC1═CC═CC(═C1O)C(═O)NCC3═NC(═C(C2═NC(C(═O)NC(C)C)═CS2)O3)C4═CC═C(F)C═C4 |
| Example 143 | OC═1C═CC═CC═1CC(═O)NCC3═NC(═C(C2═NC(C(═O)NC(C)C)═CS2)O3)C4═CC═C(F)C═C4 |
| Example 144 | C1═CC═C(C═C1O)CC(═O)NCC3═NC(═C(C2═NC(C(═O)NC(C)C)═CS2)O3)C4═CC═C(F)C═C4 |
| Example 145 | C1═CC(═C(C═C1C)C(═O)NCC3═NC(═C(C2═NC(C(═O)NC(C)C)═CS2)O3)C4═CC═C(F)C═C4)O |
| Example 146 | OC1═CC═C(O)C═C1C(═O)NCC3═NC(═C(C2═NC(C(═O)NC(C)C)═CS2)O3)C4═CC═C(F)C═C4 |
| Example 147 | OC1═C(O)C═CC═C1C(═O)NCC3═NC(═C(C2═NC(C(═O)NC(C)C)═CS2)O3)C4═CC═C(F)C═C4 |
| Example 148 | OC═4C(C(═O)NCC2═NC(═C(C1═NC(C(═O)NC(C)C)═CS1)O2)C3═CC═C(F)C═C3)═CC═C(C═4)O |
| Example 149 | FC═1C(C)═CC═C(C═1)C(═O)NCC3═NC(═C(C2═NC(C(═O)NC(C)C)═CS2)O3)C4═CC═C(F)C═C4 |
| Example 150 | CC═4C(C(═O)NCC2═NC(═C(C1═NC(C(═O)NC(C)C)═CS1)O2)C3═CC═C(F)C═C3)═CC(═CC═4)F |
| Example 151 | ClC1═CC═CC═C1C(═O)NCC3═NC(═C(C2═NC(C(═O)NC(C)C)═CS2)O3)C4═CC═C(F)C═C4 |
| Example 152 | O═C(C1═CN═C(Cl)C═C1)NCC3═NC(═C(C2═NC(C(═O)NC(C)C)═CS2)O3)C4═CC═C(F)C═C4 |
| Example 153 | ClC═1N═CC═CC═1C(═O)NCC3═NC(═C(C2═NC(C(═O)NC(C)C)═CS2)O3)C4═CC═C(F)C═C4 |
| Example 154 | ClC1═NC═CC(═C1)C(═O)NCC3═NC(═C(C2═NC(C(═O)NC(C)C)═CS2)O3)C4═CC═C(F)C═C4 |
| Example 155 | O═C(C1═NC═CC(═C1)Cl)NCC3═NC(═C(C2═NC(C(═O)NC(C)C)═CS2)O3)C4═CC═C(F)C═C4 |
| Example 156 | O═C(C1═C(C═NC═C1)Cl)NCC3═NC(═C(C2═NC(C(═O)NC(C)C)═CS2)O3)C4═CC═C(F)C═C4 |
| Example 157 | FC═1C═CC═C(F)C═1C(═O)NCC3═NC(═C(C2═NC(C(═O)NC(C)C)═CS2)O3)C4═CC═C(F)C═C4 |
| Example 158 | O═C(C1═CC═CC(F)═C1F)NCC3═NC(═C(C2═NC(C(═O)NC(C)C)═CS2)O3)C4═CC═C(F)C═C4 |
| Example 159 | O═C(C1═CC═C(F)C═C1F)NCC3═NC(═C(C2═NC(C(═O)NC(C)C)═CS2)O3)C4═CC═C(F)C═C4 |
| Example 160 | FC═1C═C(F)C═C(C═1)C(═O)NCC3═NC(═C(C2═NC(C(═O)NC(C)C)═CS2)O3)C4═CC═C(F)C═C4 |
| Example 161 | O═C(C1═CC═C(F)C(═C1)F)NCC3═NC(═C(C2═NC(C(═O)NC(C)C)═CS2)O3)C4═CC═C(F)C═C4 |
| Example 162 | O═C(CC(CC(C)(C)C)C)NC C2═NC(═C(C1═NC(C(═O)NC(C)C)═CS1)O2)C3═CC═C(F)C═C3 |
| Example 163 | C═4C5═CC═CC═C5OC═4C(═O)NCC2═NC(═C(C1═NC(C(═O)NC(C)C)═CS1)O2)C3═CC═C(F)C═C3 |
| Example 164 | O═C(C2CCC1═CC═CC═C12)NCC4═NC(═C(C3═NC(C(═O)NC(C)C)═CS3)O4)C5═CC═C(F)C═C5 |
| Example 165 | ClC(C(═O)NCC2═NC(═C(C1═NC(C(═O)NC(C)C)═CS1)O2)C3═CC═C(F)C═C3)(Cl)Cl |
| Example 166 | O═C(/C═C/C═1C(O)═CC═CC═1)NCC3═NC(═C(C2═NC(C(═O)NC(C)C)═CS2)O3)C4═CC═C(F)C═C4 |
| Example 167 | O═C(C1═CC═C(C(C)═O)C═C1)NCC3═NC(═C(C2═NC(C(═O)NC(C)C)═CS2)O3)C4═CC═C(F)C═C4 |
| Example 168 | O═C(/C═C/C1═CC(O)═CC═C1)NCC3═NC(═C(C2═NC(C(═O)NC(C)C)═CS2)O3)C4═CC═C(F)C═C4 |
| Example 169 | CC(═O)C1═CC═CC(═C1)C(═O)NCC3═NC(═C(C2═NC(C(═O)NC(C)C)═CS2)O3)C4═CC═C(F)C═C4 |
| Example 170 | CC(═O)C1═CC═CC═C1C(═O)NCC3═NC(═C(C2═NC(C(═O)NC(C)C)═CS2)O3)C4═CC═C(F)C═C4 |
| Example 171 | C4═CC(═CC═C4C(═O)NCC2═NC(═C(C1═NC(C(═O)NC(C)C)═CS1)O2)C3═CC═C(F)C═C3)C(C)C |
| Example 172 | CC═1C═CC═CC═1CCC(═O)NCC3═NC(═C(C2═NC(C(═O)NC(C)C)═CS2)O3)C4═CC═C(F)C═C4 |
| Example 173 | O═C(CCC═1C═C(C)C═CC═1)NCC3═NC(═C(C2═NC(C(═O)NC(C)C)═CS2)O3)C4═CC═C(F)C═C4 |
| Example 174 | O═C(C(CC)C1═CC═CC═C1)NCC3═NC(═C(C2═NC(C(═O)NC(C)C)═CS2)O3)C4═CC═C(F)C═C4 |
| Example 175 | CC═1C═C(C)C═C(C═1C(═O)NCC3═NC(═C(C2═NC(C(═O)NC(C)C)═CS2)O3)C4═CC═C(F)C═C4)C |
| Example 176 | C1═CC(═CC═C1CCC)C(═O)NCC3═NC(═C(C2═NC(C(═O)NC(C)C)═CS2)O3)C4═CC═C(F)C═C4 |
| Example 177 | O═C(C═1C═CC═2OCOC═2C═1)NCC4═NC(═C(C3═NC(C(═O)NC(C)C)═CS3)O4)C5═CC═C(F)C═C5 |
| Example 178 | O═C(/C═C/C═1C(F)═CC═CC═1)NCC3═NC(═C(C2═NC(C(═O)NC(C)C)═CS2)O3)C4═CC═C(F)C═C4 |
| Example 179 | C═1C═CC═C(C═1OCC)C(═O)NCC3═NC(═C(C2═NC(C(═O)NC(C)C)═CS2)O3)C4═CC═C(F)C═C4 |
| Example 180 | CC═1C═CC═CC═1OCC(═O)NCC3═NC(═C(C2═NC(C(═O)NC(C)C)═CS2)O3)C4═CC═C(F)C═C4 |
| Example 181 | O═C(CCC1═CC═CC═C1O)NCC3═NC(═C(C2═NC(C(═O)NC(C)C)═CS2)O3)C4═CC═C(F)C═C4 |
| Example 182 | O═C(CC1═C(OC)C═CC═C1)NCC3═NC(═C(C2═NC(C(═O)NC(C)C)═CS2)O3)C4═CC═C(F)C═C4 |
| Example 183 | O═C(C(C═1C═CC(O)═CC═1)C)NCC3═NC(═C(C2═NC(C(═O)NC(C)C)═CS2)O3)C4═CC═C(F)C═C4 |
| Example 184 | O═[N+]([O−])C1═CC═CC═C1C(═O)NCC3═NC(═C(C2═NC(C(═O)NC(C)C)═CS2)O3)C4═CC═C(F)C═C4 |
| Example 185 | O═[N+]([O−])C═1C═CC═C(C═1)C(═O)NCC3═NC(═C(C2═NC(C(═O)NC(C)C)═CS2)O3)C4═CC═C(F)C═C4 |
| Example 186 | COC1═C(C(O)═CC═C1)C(═O)NCC3═NC(═C(C2═NC(C(═O)NC(C)C)═CS2)O3)C4═CC═C(F)C═C4 |
| Example 187 | OC1═C(C═C(OC)C═C1)C(═O)NCC3═NC(═C(C2═NC(C(═O)NC(C)C)═CS2)O3)C4═CC═C(F)C═C4 |
| Example 188 | OC═1C═C(OC)C═CC═1C(═O)NCC3═NC(═C(C2═NC(C(═O)NC(C)C)═CS2)O3)C4═CC═C(F)C═C4 |
| Example 189 | O═C(C1═CC(O)═C(OC)C═C1)NCC3═NC(═C(C2═NC(C(═O)NC(C)C)═CS2)O3)C4═CC═C(F)C═C4 |
| Example 190 | OC═1C(O)═C(C═C(C═1)C(═O)NCC3═NC(═C(C2═NC(C(═O)NC(C)C)═CS2)O3)C4═CC═C(F)C═C4)O |
| Example 191 | FC4═CC(CC(═O)NCC2═NC(═C(C1═NC(C(═O)NC(C)C)═CS1)O2)C3═CC═C(F)C═C3)═CC═C4O |
| Example 192 | O═C(C═1C═C(N═C(C═1)Cl)C)NCC3═NC(═C(C2═NC(C(═O)NC(C)C)═CS2)O3)C4═CC═C(F)C═C4 |
| Example 193 | FC═1C═CC═C(C═1CC(═O)NCC3═NC(═C(C2═NC(C(═O)NC(C)C)═CS2)O3)C4═CC═C(F)C═C4)F |
| Example 194 | O═C(C2═CC═CC1═CC═CC═C12)NCC4═NC(═C(C3═NC(C(═O)NC(C)C)═CS3)O4)C5═CC═C(F)C═C5 |
| Example 195 | O═C(C1═CC═C(Cl)C═C1O)NCC3═NC(═C(C2═NC(C(═O)NC(C)C)═CS2)O3)C4═CC═C(F)C═C4 |
| Example 196 | OC═4C(C(═O)NCC2═NC(═C(C1═NC(C(═O)NC(C)C)═CS1)O2)C3═CC═C(F)C═C3)═CC(═CC═4)Cl |
| Example 197 | O═C(C1═CC═2C═CC═NC═2C═C1)NCC4═NC(═C(C3═NC(C(═O)NC(C)C)═CS3)O4)C5═CC═C(F)C═C5 |
| Example 198 | OC4═NC═C(C(═O)NCC2═NC(═C(C1═NC(C(═O)NC(C)C)═CS1)O2)C3═CC═C(F)C═C3)C═C4Cl |
| Example 199 | O═C(C1═CC═C(F)C═C1Cl)NCC3═NC(═C(C2═NC(C(═O)NC(C)C)═CS2)O3)C4═CC═C(F)C═C4 |
| Example 200 | FC═1C═CC═C(C═1C(═O)NCC3═NC(═C(C2═NC(C(═O)NC(C)C)═CS2)O3)C4═CC═C(F)C═C4)Cl |
| Example 201 | CC(C)(OC(NCC(═O)NCC2═NC(═C(C1═NC(C(═O)NC(C)C)═CS1)O2)C3═CC═C(F)C═C3)═O)C |
| Example 202 | O═C(C═1OC2═CC═CC═C2C═1C)NCC4═NC(═C(C3═NC(C(═O)NC(C)C)═CS3)O4)C5═CC═C(F)C═C5 |
| Example 203 | C═1C2═C(C═CC═1)CC(C2)CC(═O)NCC4═NC(═C(C3═NC(C(═O)NC(C)C)═CS3)O4)C5═CC═C(F)C═C5 |
| Example 204 | C4CC5═C(CC4C(═O)NCC2═NC(═C(C1═NC(C(═O)NC(C)C)═CS1)O2)C3═CC═C(F)C═C3)C═CC═C5 |
| Example 205 | O═C(/C═C/C═1C═CC═CC═1OC)NCC3═NC(═C(C2═NC(C(═O)NC(C)C)═CS2)O3)C4═CC═C(F)C═C4 |
| Example 206 | O═C(C1═CSC2═C1C═CC═C2)NCC4═NC(═C(C3═NC(C(═O)NC(C)C)═CS3)O4)C5═CC═C(F)C═C5 |
| Example 207 | C═1C═C2C═C(SC2═CC═1)C(═O)NCC4═NC(═C(C3═NC(C(═O)NC(C)C)═CS3)O4)C5═CC═C(F)C═C5 |
| Example 208 | O═C(C1═CC═C(CCCC)C═C1)NCC3═NC(═C(C2═NC(C(═O)NC(C)C)═CS2)O3)C4═CC═C(F)C═C4 |
| Example 209 | O═C(C1═CC═C(C(C)(C)C)C═C1)NCC3═NC(═C(C2═NC(C(═O)NC(C)C)═CS2)O3)C4═CC═C(F)C═C4 |
| Example 210 | O═C(C1═CC═C(N(C═O)C)C═C1)NCC3═NC(═C(C2═NC(C(═O)NC(C)C)═CS2)O3)C4═CC═C(F)C═C4 |
| Example 211 | O═C(C═1C═C(NC(C)═O)C═CC═1)NCC3═NC(═C(C2═NC(C(═O)NC(C)C)═CS2)O3)C4═CC═C(F)C═C4 |
| Example 212 | O═C(COC═1C(C═O)═CC═CC═1)NCC3═NC(═C(C2═NC(C(═O)NC(C)C)═CS2)O3)C4═CC═C(F)C═C4 |
| Example 213 | CC(═O)OC1═CC═C(C═C1)C(═O)NCC3═NC(═C(C2═NC(C(═O)NC(C)C)═CS2)O3)C4═CC═C(F)C═C4 |
| Example 214 | O═C(OC)O4═CC═O(C(═O)NCC2═NC(═C(C1═NC(C(═O)NC(C)C)═CS1)O2)C3═CC═C(F)C═C3)C═C4 |
| Example 215 | C4═C(C═CC═C4C(═O)NCC2═NC(═C(C1═NC(C(═O)NC(C)C)═CS1)O2)C3═CC═C(F)C═C3)OC(═O)C |
| Example 216 | CC(═O)OC1═CC═CC═C1C(═O)NCC3═NC(═C(C2═NC(C(═O)NC(C)C)═CS2)O3)C4═CC═C(F)C═C4 |
| Example 217 | O1C═2C═CC(═CC═2OC1)CC(═O)NCC4═NC(═C(C3═NC(C(═O)NC(C)C)═CS3)O4)C5═CC═C(F)C═C5 |
| Example 218 | C1═CC(═CC═C1OCCC)C(═O)NCC3═NC(═C(C2═NC(C(═O)NC(C)C)═CS2)O3)C4═CC═C(F)C═C4 |
| Example 219 | O═C(C1═CC═C(OC(C)C)C═C1)NCC3═NC(═C(C2═NC(C(═O)NC(C)C)═CS2)O3)C4═CC═C(F)C═C4 |
| Example 220 | [O−][N+](═O)C1═C(C(═CC═C1)C)C(═O)NCC3═NC(═C(C2═NC(C(═O)NC(C)C)═CS2)O3)C4═CC═C(F)C═C4 |
| Example 221 | O═[N+]([O−])C1═C(C═CC═C1C(═O)NCC3═NC(═C(C2═NC(C(═O)NC(C)C)═CS2)O3)C4═CC═C(F)C═C4)C |
| Example 222 | [O−][N+](═O)C1═CC═C(C═C1C(═O)NCC3═NC(═C(C2═NC(C(═O)NC(C)C)═CS2)O3)C4═CC═C(F)C═C4)C |
| Example 223 | CC═4C(C(═O)NCC2═NC(═C(C1═NC(C(═O)NC(C)C)═CS1)O2)C3═CC═C(F)C═C3)═CC═CC═4[N+]([O−])═O |
| Example 224 | C1═CC(═C(C═C1OC)C(═O)NCC3═NC(═C(C2═NC(C(═O)NC(C)C)═CS2)O3)C4═CC═C(F)C═C4)OC |
| Example 225 | COC1═CC(OC)═CC═C1C(═O)NCC3═NC(═C(C2═NC(C(═O)NC(C)C)═CS2)O3)C4═CC═C(F)C═C4 |
| Example 226 | COC═1C(═C(OC)C═CC═1)C(═O)NCC3═NC(═C(C2═NC(C(═O)NC(C)C)═CS2)O3)C4═CC═C(F)C═C4 |
| Example 227 | C═1C(═CC(═CC═1OC)C(═O)NCC3═NC(═C(C2═NC(C(═O)NC(C)C)═CS2)O3)C4═CC═C(F)C═C4)OC |
| Example 228 | C═1C═CC(═C(C═1OC)OC)C(═O)NCC3═NC(═C(C2═NC(C(═O)NC(C)C)═CS2)O3)C4═CC═C(F)C═C4 |
| Example 229 | CC4═CC═C(SCC(═O)NCC2═NC(═C(C1═NC(C(═O)NC(C)C)═CS1)O2)C3═CC═C(F)C═C3)C═C4 |
| Example 230 | O═C(/C═C/C═1C═CC(Cl)═CC═1)NCC3═NC(═C(C2═NC(C(═O)NC(C)C)═CS2)O3)C4═CC═C(F)C═C4 |
| Example 231 | O═C(/C═C/C═1C(Cl)═CC═CC═1)NCC3═NC(═C(C2═NC(C(═O)NC(C)C)═CS2)O3)C4═CC═C(F)C═C4 |
| Example 232 | [O−][N+](C1═CC═C(C═C1O)C(═O)NCC3═NC(═C(C2═NC(C(═O)NC(C)C)═CS2)O3)C4═CC═C(F)C═C4)═O |
| Example 233 | O═C(C1═CC(O)═CC═C1[N+]([O−])═O)NCC3═NC(═C(C2═NC(C(═O)NC(C)C)═CS2)O3)C4═CC═C(F)C═C4 |
| Example 234 | OC4═CC═C(C═C4C(═O)NCC2═NC(═C(C1═NC(C(═O)NC(C)C)═CS1)O2)C3═CC═C(F)C═C3)[N+](═O)[O−] |
| Example 235 | O═[N+]([O−])C1═CC═C(C(═C1)C(═O)NCC3═NC(═C(C2═NC(C(═O)NC(C)C)═CS2)O3)C4═CC═C(F)C═O4)F |
| Example 236 | O═[N+]([O−])C1═CC═C(C═C1C(═O)NCC3═NC(═C(C2═NC(C(═O)NC(C)C)═CS2)O3)C4═CC═C(F)C═C4)F |
| Example 237 | O═[N+]([O−])C═1C(═CC═C(C═1)C(═O)NCC3═NC(═C(C2═NC(C(═O)NC(C)C)═CS2)O3)C4═CC═C(F)C═C4)F |
| Example 238 | COC1═CC═C(Cl)C═C1C(═O)NCC3═NC(═C(C2═NC(C(═O)NC(C)C)═CS2)O3)C4═CC═C(F)C═C4 |
| Example 239 | ClC═1C═C(OC)C(═CC═1)C(═O)NCC3═NC(═C(C2═NC(C(═O)NC(C)C)═CS2)O3)C4═CC═C(F)C═C4 |
| Example 240 | O═C(CC1═CC(═C(O)C═C1)Cl)NCC3═NC(═C(C2═NC(C(═O)NC(C)C)═CS2)O3)C4═CC═C(F)C═C4 |
| Example 241 | O═C(C═1C═C(N═C(C═1)Cl)OC)NCC3═NC(═C(C2═NC(C(═O)NC(C)C)═CS2)O3)C4═CC═C(F)C═C4 |
| Example 242 | OC5═CC═C1C(C═CC(═C1)C(═O)NCC3═NC(═C(C2═NC(C(═O)NC(C)C)═CS2)O3)C4═CC═C(F)C═C4)═C5 |
| Example 243 | O═C(C═1C2═CC═CC═C2C═CC═1O)NCC4═NC(═C(C3═NC(C(═O)NC(C)C)═CS3)O4)C5═CC═C(F)C═C5 |
| Example 244 | O═C(C1═CC2═CC═CC═C2C═C1O)NCC4═NC(═C(C3═NC(C(═O)NC(C)C)═CS3)O4)C5═CC═C(F)C═C5 |
| Example 245 | C1═CC═2C(C═C1)═CC═C(C═2O)C(═O)NCC4═NC(═C(C3═NC(C(═O)NC(C)C)═CS3)O4)C5═CC═C(F)C═C5 |
| Example 246 | OC1═CC═CC2═CC═C(N═C12)C(═O)NCC4═NC(═C(C3═NC(C(═O)NC(C)C)═CS3)O4)C5═CC═C(F)C═C5 |
| Example 247 | CC(C)(OC(NC(C(═O)NCC2═NC(═C(C1═NC(C(═O)NC(C)C)═CS1)O2)C3═CC═C(F)C═C3)C)═O)C |
| Example 248 | CC(C)(OC(NC(C(═O)NCC2═NC(═C(C1═NC(C(═O)NC(C)C)═CS1)O2)C3═CC═C(F)C═C3)C)═O)C |
| Example 249 | O═C(C═1C(═CC═CC═1)C(F)(F)F)NCC3═NC(═C(C2═NC(C(═O)NC(C)C)═CS2)O3)C4═CC═C(F)C═C4 |
| Example 250 | [H]N2C1═CC═CC═C1N═C2CCC(═O)NCC4═NC(═C(C3═NC(C(═O)NC(C)C)═CS3)O4)C5═CC═C(F)C═C5 |
| Example 251 | ClC1═C(Cl)C═CC═C1C(═O)NCC3═NC(═C(C2═NC(C(═O)NC(C)C)═CS2)O3)C4═CC═C(F)C═C4 |
| Example 252 | ClC1═CC(Cl)═CC═C1C(═O)NCC3═NC(═C(C2═NC(C(═O)NC(C)C)═CS2)O3)C4═CC═C(F)C═C4 |
| Example 253 | ClC═4C(C(═O)NCC2═NC(═C(C1═NC(C(═O)NC(C)C)═CS1)O2)C3═CC═C(F)C═C3)═CC(═CC═4)Cl |
| Example 254 | O═C(C1═C(C(═C(C(═C1)F)F)O)F)NCC3═NC(═C(C2═NC(C(═O)NC(C)C)═CS2)O3)C4═CC═C(F)C═C4 |
| Example 255 | O1C═2C═CC(═CC═2OC1)/ C═C/C(═O)NCC4═NC(═C(C3═NC(C(═O)NC(C)C)═CS3)O4)C5═CC═C(F)C═C5 |
| Example 256 | C1═CC(═CC═C1C)C(CCC(═O)NCC3═NC(═C(C2═NC(C(═O)NC(C)C)═CS2)O3)C4═CC═C(F)C═C4)═O |
| Example 257 | O═C(C1═CC═C(CCCCC)C═C1)NCC3═NC(═C(C2═NC(C(═O)NC(C)C)═CS2)O3)C4═CC═C(F)C═C4 |
| Example 258 | O═C(C1═CC(═C(F)C═C1Cl)F)NCC3═NC(═C(C2═NC(C(═O)NC(C)C)═CS2)O3)C4═CC═C(F)C═C4 |
| Example 259 | FC1═CC(Cl)═C(C═C1C(═O)NCC3═NC(═C(C2═NC(C(═O)NC(C)C)═CS2)O3)C4═CC═C(F)C═C4)F |
| Example 260 | O═C(C1═CC═C(N(CC)CC)C═C1)NCC3═NC(═C(C2═NC(C(═O)NC(C)C)═CS2)O3)C4═CC═C(F)C═C4 |
| Example 261 | COC4═CC(/C═C/C(═O)NCC2═NC(═C(C1═NC(C(═O)NC(C)C)═CS1)O2)C3═CC═C(F)C═C3)═CC═C4O |
| Example 262 | O═C(/C═C/C1═CC(O)═C(OC)C═C1)NCC3═NC(═C(C2═NC(C(═O)NC(C)C)═CS2)O3)C4═CC═C(F)C═C4 |
| Example 263 | O═C(C1═CC═C(OCCCC)C═C1)NCC3═NC(═C(C2═NC(C(═O)NC(C)C)═CS2)O3)C4═CC═C(F)C═C4 |
| Example 264 | O═C(C═2OC1═C(C═C(Cl)C═C1)C═2)NCC4═NC(═C(C3═NC(C(═O)NC(C)C)═CS3)O4)C5═CC═C(F)C═C5 |
| Example 265 | C1═CC(═C(C═C1OC)C(═O)NCC3═NC(═C(C2═NC(C(═O)NC(C)C)═CS2)O3)C4═CC═C(F)C═C4)[N+]([O−])═O |
| Example 266 | O═C(C1═CC(OC)═C(O)C(OC)═C1)NCC3═NC(═C(C2═NC(C(═O)NC(C)C)═CS2)O3)C4═CC═C(F)C═C4 |
| Example 267 | C5═CC═CC═C5C═4C(C(═O)NCC2═NC(═C(C1═NC(C(═O)NC(C)C)═CS1)O2)C3═CC═C(F)C═C3)═CC═CC═4 |
| Example 268 | BrC1═CC═CC(═C1)C(═O)NCC3═NC(═C(C2═NC(C(═O)NC(C)C)═CS2)O3)C4═CC═C(F)C═C4 |
| Example 269 | C═1C═C(C═CC═1Br)C(═O)NCC3═NC(═C(C2═NC(C(═O)NC(C)C)═CS2)O3)C4═CC═C(F)C═C4 |
| Example 270 | [O−][N+](═O)C1═C(C═CC═C1C(═O)NCC3═NC(═C(C2═NC(C(═O)NC(C)C)═CS2)O3)C4═CC═C(F)C═C4)Cl |
| Example 271 | O═[N+]([O−])C1═CC═C(C(═C1)C(═O)NCC3═NC(═C(C2═NC(C(═O)NC(C)C)═CS2)O3)C4═CC═C(F)C═C4)Cl |
| Example 272 | O═C(C1═C(C═C([N+]([O−])═O)C═C1)Cl)NCC3═NC(═C(C2═NC(C(═O)NC(C)C)═CS2)O3)C4═CC═C(F)C═C4 |
| Example 273 | O═[N+]([O−])C1═CC(═CC═C1C(═O)NCC3═NC(═C(C2═NC(C(═O)NC(C)C)═CS2)O3)C4═CC═C(F)C═C4)Cl |
| Example 274 | O═[N+]([O−])C1═CC(═CC═C1Cl)C(═O)NCC3═NC(═C(C2═NC(C(═O)NC(C)C)═CS2)O3)C4═CC═C(F)C═C4 |
| Example 275 | O═C(C1═CC(Cl)═CC═C1[N+](═O)[O−])NCC3═NC(═C(C2═NC(C(═O)NC(C)C)═CS2)O3)C4═CC═C(F)C═C4 |
| Example 276 | C═4C═CC(═NC═4C(═O)NCC2═NC(═C(C1═NC(C(═O)NC(C)C)═CS1)O2)C3═CC═C(F)C═C3)Br |
| Example 277 | O═C(C═1C═NC═C(Br)C═1)NCC3═NC(═C(C2═NC(C(═O)NC(C)C)═CS2)O3)C4═CC═C(F)C═C4 |
| Example 278 | [O−][N+](═O)C═4C(C(═O)NCC2═NC(═C(C1═NC(C(═O)NC(C)C)═CS1)O2)C3═CC═C(F)C═C3)═CC(F)═C(F)C═4 |
| Example 279 | FC(F)(C═1C═CC(═CC═1)CC(═O)NCC3═NC(═C(C2═NC(C(═O)NC(C)C)═CS2)O3)C4═CC═C(F)C═C4)F |
| Example 280 | CC(C)CC4═CC═C(C(C(═O)NCC2═NC(═C(C1═NC(C(═O)NC(C)C)═CS1)O2)C3═CC═C(F)C═C3)C)C═C4 |
| Example 281 | ClC1═C(O)C(═CC(═C1)C(═O)NCC3═NC(═C(C2═NC(C(═O)NC(C)C)═CS2)O3)C4═CC═C(F)C═C4)Cl |
| Example 282 | FC═1C(F)═C(C(═C(C═1F)C(═O)NCC3═NC(═C(C2═NC(C(═O)NC(C)C)═CS2)O3)C4═CC═C(F)C═C4)F)F |
| Example 283 | O═C(C1═C(OC)C═C(C═C1OC)OC)NCC3═NC(═C(C2═NC(C(═O)NC(C)C)═CS2)O3)C4═CC═C(F)C═C4 |
| Example 284 | COC1═CC(═C(C═C1OC)OC)C(═O)NCC3═NC(═C(C2═NC(C(═O)NC(C)C)═CS2)O3)C4═CC═C(F)C═C4 |
| Example 285 | COC1═C(OC)C(═CC(═C1)C(═O)NCC3═NC(═C(C2═NC(C(═O)NC(C)C)═CS2)O3)C4═CC═C(F)C═C4)OC |
| Example 286 | COC1═C(OC)C═CC(═C1OC)C(═O)NCC3═NC(═C(C2═NC(C(═O)NC(C)C)═CS2)O3)C4═CC═C(F)C═C4 |
| Example 287 | C═1C═C(C═CC═1)CC5═C(C(═O)NCC3═NC(═C(C2═NC(C(═O)NC(C)C)═CS2)O3)C4═CC═C(F)C═C4)C═CC═C5 |
| Example 288 | O═C(C2═CC═CC(OC1═CC═CC═C1)═C2)NCC4═NC(═C(C3═NC(C(═O)NC(C)C)═CS3)O4)C5═CC═C(F)C═C5 |
| Example 289 | C1═CC(═CC═C1)OC5═C(C(═O)NCC3═NC(═C(C2═NC(C(═O)NC(C)C)═CS2)O3)C4═CC═C(F)C═C4)C═CC═C5 |
| Example 290 | O═C(C2═CC═C(OC1═CC═CC═C1)C═C2)NCC4═NC(═C(C3═NC(C(═O)NC(C)C)═CS3)O4)C5═CC═C(F)C═C5 |
| Example 291 | C1═CC(═CC═C1O)C2═CC═C(C═C2)C(═O)NCC4═NC(═C(C3═NC(C(═O)NC(C)C)═CS3)O4)C5═CC═C(F)C═C5 |
| Example 292 | BrC═1C═CC(═CC═1C)C(═O)NCC3═NC(═C(C2═NC(C(═O)NC(C)C)═CS2)O3)C4═CC═C(F)C═C4 |
| Example 293 | BrC1═C(C)C═CC(═C1)C(═O)NCC3═NC(═C(C2═NC(C(═O)NC(C)C)═CS2)O3)C4═CC═C(F)C═C4 |
| Example 294 | O═C(CC1═CC═C(Br)C═C1)NCC3═NC(═C(C2═NC(C(═O)NC(C)C)═CS2)O3)C4═CC═C(F)C═C4 |
| Example 295 | CC(OC(═O)N1C(CCC1)C(═O)NCC3═NC(═C(C2═NC(C(═O)NC(C)C)═CS2)O3)C4═CC═C(F)C═C4)(C)C |
| Example 296 | O═C(/C═C/C1═C(C(F)(F)F)C═CC═C1)NCC3═NC(═C(C2═NC(C(═O)NC(C)C)═CS2)O3)C4═CC═C(F)C═C4 |
| Example 297 | O═C(C1═C(OCC)C═CC2═C1C═CC═C2)NCC4═NC(═C(C3═NC(C(═O)NC(C)C)═CS3)O4)C5═CC═C(F)C═C5 |
| Example 298 | CC(C)(OC(NC(C(═O)NCC2═NC(═C(C1═NC(C(═O)NC(C)C)═CS1)O2)C3═CC═C(F)C═C3)C(C)C)═O)C |
| Example 299 | CC(C)(OC(NC(C(═O)NCC2═NC(═C(C1═NC(C(═O)NC(C)C)═CS1)O2)C3═CC═C(F)C═C3)C(C)C)═O)C |
| Example 300 | O═C(CCC═1C═C(C═CC═1)C(F)(F)F)NCC3═NC(═C(C2═NC(C(═O)NC(C)C)═CS2)O3)C4═CC═C(F)C═C4 |
| Example 301 | O═C(C═2C═CC(N1N═C(C)CC1═O)═CC═2)NCC4═NC(═C(C3═NC(C(═O)NC(C)C)═CS3)O4)C5═CC═C(F)C═C5 |
| Example 302 | CC4═C(C(═O)NCC2═NC(═C(C1═NC(C(═O)NC(C)C)═CS1)O2)C3═CC═C(F)C═C3)SC(═N4)Br |
| Example 303 | C1═CC2═C(C═C1)C═C6C(═C2C(═O)NCC4═NC(═C(C3═NC(C(═O)NC(C)C)═CS3)O4)C5═CC═C(F)C═C5)C═CC═C6 |
| Example 304 | CC(C)C4═CC(C(═O)NCC2═NC(═C(C1═NC(C(═O)NC(C)C)═CS1)O2)C3═CC═C(F)C═C3)═C(C(═C4)C(C)C)O |
| Example 305 | CC(═O)NC(C(═O)NCC2═NC(═C(C1═NC(C(═O)NC(C)C)═CS1)O2)C3═CC═C(F)C═C3)CC4═CC═C(C═C4)O |
| Example 306 | ClC1═CC(Cl)═CC(═C1C(═O)NCC3═NC(═C(C2═NC(C(═O)NC(C)C)═CS2)O3)C4═CC═C(F)C═C4)Cl |
| Example 307 | COC1═CC(═C(C═C1OC)[N+](═O)[O−])C(═O)NCC3═NC(═C(C2═NC(C(═O)NC(C)C)═CS2)O3)C4═CC═C(F)C═C4 |
| Example 308 | O═C(C2═CC═C(OCC1═CC═CC═C1)C═C2)NCC4═NC(═C(C3═NC(C(═O)NC(C)C)═CS3)O4)C5═CC═C(F)C═C5 |
| Example 309 | BrC═4C═CC═CC═4CCC(═O)NCC2═NC(═C(C1═NC(C(═O)NC(C)C)═CS1)O2)C3═CC═C(F)C═C3 |
| Example 310 | O═C(CCC═1C═C(Br)C═CC═1)NCC3═NC(═C(C2═NC(C(═O)NC(C)C)═CS2)O3)C4═CC═C(F)C═C4 |
| Example 311 | CC(OC(N4C(C(═O)NCC2═NC(═C(C1═NC(C(═O)NC(C)C)═CS1)O2)C3═CC═C(F)C═C3)CCCC4)═O)(C)C |
| Example 312 | C5═CC═C(C═C5OC═1C═CC(═CC═1)C(═O)NCC3═NC(═C(C2═NC(C(═O)NC(C)C)═CS2)O3)C4═CC═C(F)C═C4)O |
| Example 313 | CC(C(═O)NCC2═NC(═C(C1═NC(C(═O)NC(C)C)═CS1)O2)C3═CC═C(F)C═C3)C═5C═CC4═CC(═CC═C4C═5)OC |
| Example 314 | CC(C)(OC(N1CC(NCC1)C(═O)NCC3═NC(═C(C2═NC(C(═O)NC(C)C)═CS2)O3)C4═CC═C(F)C═C4)═O)C |
| Example 315 | CC(C)(OC(N1CC(NCC1)C(═O)NCC3═NC(═C(C2═NC(C(═O)NC(C)C)═CS2)O3)C4═CC═C(F)C═C4)═O)C |
| Example 316 | COC1═CC(═C(C═C1)Br)C(═O)NCC3═NC(═C(C2═NC(C(═O)NC(C)C)═CS2)O3)C4═CC═C(F)C═C4 |
| Example 317 | CC(OC(═O)N1CC(CC1C(═O)NCC3═NC(═C(C2═NC(C(═O)NC(C)C)═CS2)O3)C4═CC═C(F)C═C4)O)(C)C |
| Example 318 | CC(C)C(C(═O)NCC2═NC(═C(C1═NC(C(═O)NC(C)C)═CS1)O2)C3═CC═C(F)C═C3)N(C(OC(C)(C)C)═O)C |
| Example 319 | FC(F)(C4═CC([N+]([O−])═O)═C(C(═O)NCC2═NC(═C(C1═NC(C(═O)NC(C)C)═CS1)O2)C3═CC═C(F)C═C3)C═C4)F |
| Example 320 | BrC1═CC═C(Cl)C(═C1)C(═O)NCC3═NC(═C(C2═NC(C(═O)NC(C)C)═CS2)O3)C4═CC═C(F)C═C4 |
| Example 321 | O═C(C═1C═C(Br)C(═CC═1)Cl)NCC3═NC(═C(C2═NC(C(═O)NC(C)C)═CS2)O3)C4═CC═C(F)C═C4 |
| Example 322 | C═1C═C(C═CC═1O)C(═O)C2═CC═CC═C2C(═O)NCC4═NC(═C(C3═NC(C(═O)NC(C)C)═CS3)O4)C5═CC═C(F)C═C5 |
| Example 323 | C1═CC(═CC═C1F)C(═O)C5═C(C(═O)NCC3═NC(═C(C2═NC(C(═O)NC(C)C)═CS2)O3)C4═CC═C(F)C═C4)C═CC═C5 |
| Example 324 | O═C(C(C2═CC(═C(C1═CC═CC═C1)C═C2)F)C)NCC4═NC(═C(C3═NC(C(═O)NC(C)C)═CS3)O4)C5═CC═C(F)C═C5 |
| Example 325 | C═1C═C(C═CC═1l)C(═O)NCC3═NC(═C(C2═NC(C(═O)NC(C)C)═CS2)O3)C4═CC═C(F)C═C4 |
| Example 326 | lC1═CC═CC(═C1)C(═O)NCC3═NC(═C(C2═NC(C(═O)NC(C)C)═CS2)O3)C4═CC═C(F)C═C4 |
| Example 327 | O═C(OCC1═CC═CC═C1)N2C(CCC2)C(═O)NCC4═NC(═C(C3═NC(C(═O)NC(C)C)═CS3)O4)C5═CC═C(F)C═C5 |
| Example 328 | CC(CC)C(C(═O)NCC2═NC(═C(C1═NC(C(═O)NC(C)C)═CS1)O2)C3═CC═C(F)C═C3)NC(OC(C)(C)C)═O |
| Example 329 | CC(C)(OC(NC(C(═O)NCC2═NC(═C(C1═NC(C(═O)NC(C)C)═CS1)O2)C3═CC═C(F)C═C3)CC(C)C)═O)C |
| Example 330 | O═C(C1═CC═CC═C1)C═2C═C(C═CC═2)C(C)C(═O)NCC4═NC(═C(C3═NC(C(═O)NC(C)C)═CS3)O4)C5═CC═C(F)C═C5 |
| Example 331 | CC(C)(OC(NC(C(═O)NCC2═NC(═C(C1═NC(C(═O)NC(C)C)═CS1)O2)C3═CC═C(F)C═C3)CC4═CC═CC═C4)═O)C |
| Example 332 | CC(C)(OC(NC(C(═O)NCC2═NC(═C(C1═NC(C(═O)NC(C)C)═CS1)O2)C3═CC═C(F)C═C3)CC4═NC═CC═C4)═O)C |
| Example 333 | CC(OC(═O)N2CC═1C═CC═CC═1CC2C(═O)NCC4═NC(═C(C3═NC(C(═O)NC(C)C)═CS3)O4)C5═CC═C(F)C═C5)(C)C |
| Example 334 | C1═CC(═CC═C1O)CC(C(═O)NCC3═NC(═C(C2═NC(C(═O)NC(C)C)═CS2)O3)C4═CC═C(F)C═C4)NC(OC(C)(C)C)═O |

### EXAMPLES 335-572

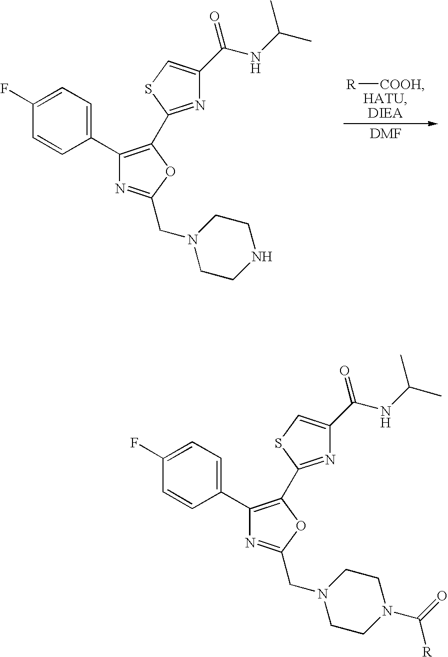

Starting amino methyl oxazole was prepared as described in Example 44. Where R—COOH is a carboxylic acid selected to afford Examples 335-572, which were prepared by General Procedure 1.

| | |
|---|---|
| Example 335 | CC(═O)N4CCN(CC═2OC(C═1SC═C(C(═O)NC(C)C)N═1)═C(N═2)C3═CC═C(F)C═C3)CC4 |
| Example 336 | O═C(C═O)N4CCN(CC═2OC(C═1SC═C(C(═O)NC(C)C)N═1)═C(N═2)C3═CC═C(F)C═C3)CC4 |
| Example 337 | O═C(CC#N)N4CCN(CC═2OC(C═1SC═C(C(═O)NC(C)C)N═1)═C(N═2)C3═CC═C(F)C═C3)CC4 |
| Example 338 | OC(C)C(═O)N4CCN(CC═2OC(C═1SC═C(C(═O)NC(C)C)N═1)═C(N═2)C3═CC═C(F)C═C3)CC4 |
| Example 339 | O═C(CC)C(═O)N4CCN(CC═2OC(C═1SC═C(C(═O)NC(C)C)N═1)═C(N═2)C3═CC═C(F)C═C3)CC4 |
| Example 340 | O═C(C(C)(C)C)N4CCN(CC═2OC(C═1SC═C(C(═O)NC(C)C)N═1)═C(N═2)C3═CC═C(F)C═C3)CC4 |
| Example 341 | O═C(CC(C)C)N4CCN(CC═2OC(C═1SC═C(C(═O)NC(C)C)N═1)═C(N═2)C3═CC═C(F)C═C3)CC4 |
| Example 342 | [H]N5C(C(═O)N4CCN(CC═2OC(C═1SC═C(C(═O)NC(C)C)N═1)═C(N═2)C3═CC═C(F)C═C3)CC4)═CN═C5 |
| Example 343 | [H]N1C(═NC═C1)C(═O)N5CCN(CC═3OC(C═2SC═C(C(═O)NC(C)C)N═2)═C(N═3)C4═CC═C(F)C═C4)CC5 |
| Example 344 | C1CC(═CC1)C(═O)N5CCN(CC═3OC(C═2SC═C(C(═O)NC(C)C)N═2)═C(N═3)C4═CC═C(F)C═C4)CC5 |
| Example 345 | FC(F)(C(═O)N4CCN(CC═2OC(C═1SC═C(C(═O)NC(C)C)N═1)═C(N═2)C3═CC═C(F)C═C3)CC4)F |
| Example 346 | C5CCOC5C(═O)N4CCN(CC═2OC(C═1SC═C(C(═O)NC(C)C)N═1)═C(N═2)C3═CC═C(F)C═C3)CC4 |
| Example 347 | O═C(C)NCC(═O)N4CCN(CC═2OC(C═1SC═C(C(═O)NC(C)C)N═1)═C(N═2)C3═CC═C(F)C═C3)CC4 |
| Example 348 | C═5C═CC═CC═5C(═O)N4CCN(CC═2OC(C═1SC═C(C(═O)NC(C)C)N═1)═C(N═2)C3═CC═C(F)C═C3)CC4 |
| Example 349 | C═5C═CN═CC═5C(═O)N4CCN(CC═2OC(C═1SC═C(C(═O)NC(C)C)N═1)═C(N═2)C3═CC═C(F)C═C3)CC4 |
| Example 350 | C1═CC(═CC═N1)C(═O)N5CCN(CC═3OC(C═2SC═C(C(═O)NC(C)C)N═2)═C(N═3)C4═CC═C(F)C═C4)CC5 |
| Example 351 | C1═NC(═CC═C1)C(═O)N5CCN(CC═3OC(C═2SC═C(C(═O)NC(C)C)N═2)═C(N═3)C4═CC═C(F)C═C4)CC5 |
| Example 352 | OC(CCl)C(═O)N4CCN(CC═2OC(C═1SC═C(C(═O)NC(C)C)N═1)═C(N═2)C3═CC═C(F)C═C3)CC4 |
| Example 353 | [H]N1N═C(C═C1C)C(═O)N5CCN(CC═3OC(C═2SC═C(C(═O)NC(C)C)N═2)═C(N═3)C4═CC═C(F)C═C4)CC5 |
| Example 354 | O═C(C═1N═CN(C═1)C)N5CCN(CC═3OC(C═2SC═C(C(═O)NC(C)C)N═2)═C(N═3)C4═CC═C(F)C═C4)CC5 |
| Example 355 | C1CC(CCC1)C(═O)N5CCN(CC═3OC(C═2SC═C(C(═O)NC(C)C)N═2)═C(N═3)C4═CC═C(F)C═C4)CC5 |
| Example 356 | N5═CSC═C5C(═O)N4CCN(CC═2OC(C═1SC═C(C(═O)NC(C)C)N═1)═C(N═2)C3═CC═C(F)C═C3)CC4 |
| Example 357 | O═C(OCC)CC(═O)N4CCN(CC═2OC(C═1SC═C(C(═O)NC(C)C)N═1)═C(N═2)C3═CC═C(F)C═C3)CC4 |
| Example 358 | CC5═CC(C(═O)N4CCN(CC═2OC(C═1SC═C(C(═O)NC(C)C)N═1)═C(N═2)C3═CC═C(F)C═C3)CC4)═CC═C5 |
| Example 359 | O═C(C1═CC═CC═C1C)N5CCN(CC═3OC(C═2SC═C(C(═O)NC(C)C)N═2)═C(N═3)C4═CC═C(F)C═C4)CC5 |
| Example 360 | O═C(C1═CC═C(C)C═C1)N5CCN(CC═3OC(C═2SC═C(C(═O)NC(C)C)N═2)═C(N═3)C4═CC═C(F)C═C4)CC5 |
| Example 361 | OC═1C═CC═C(C═1)C(═O)N5CCN(CC═3OC(C═2SC═C(C(═O)NC(C)C)N═2)═C(N═3)C4═CC═C(F)C═C4)CC5 |
| Example 362 | OC═5C(C(═O)N4CCN(CC═2OC(C═1SC═C(C(═O)NC(C)C)N═1)═C(N═2)C3═CC═C(F)C═C3)CC4)═CC═CC═5 |
| Example 363 | C═1C═C(C═CC═1F)C(═O)N5CCN(CC═3OC(C═2SC═C(C(═O)NC(C)C)N═2)═C(N═3)C4═CC═C(F)C═C4)CC5 |
| Example 364 | C1═CC═C(C═C1F)C(═O)N5CCN(CC═3OC(C═2SC═C(C(═O)NC(C)C)N═2)═C(N═3)C4═CC═C(F)C═C4)CC5 |
| Example 365 | CCCCCCCC(═O)N4CCN(CC═2OC(C═1SC═C(C(═O)NC(C)C)N═1)═C(N═2)C3═CC═C(F)C═C3)CC4 |
| Example 366 | O═C(C1═CC═C(C#N)C═C1)N5CCN(CC═3OC(C═2SC═C(C(═O)NC(C)C)N═2)═C(N═3)C4═CC═C(F)C═C4)CC5 |
| Example 367 | O═C(C1═CC═CC(C#N)═C1)N5CCN(CC═3OC(C═2SC═C(C(═O)NC(C)C)N═2)═C(N═3)C4═CC═C(F)C═C4)CC5 |
| Example 368 | C═5C═CC═CC═5/C═C/C(═O)N4CCN(CC═2OC(C═1SC═C(C(═O)NC(C)C)N═1)═C(N═2)C3═CC═C(F)C═C3)CC4 |
| Example 369 | O═C(C1═CC═CC═C1C═O)N5CCN(CC═3OC(C═2SC═C(C(═O)NC(C)C)N═2)═C(N═3)C4═CC═C(F)C═C4)CC5 |
| Example 370 | O═C(C1═CC═C(C═O)C═C1)N5CCN(CC═3OC(C═2SC═C(C(═O)NC(C)C)N═2)═C(N═3)C4═CC═C(F)C═C4)CC5 |
| Example 371 | CC1═CC(C)═CC═C1C(═O)N5CCN(CC═3OC(C═2SC═C(C(═O)NC(C)C)N═2)═C(N═3)C4═CC═C(F)C═C4)CC5 |
| Example 372 | C═1C═CC═C(C═1C)CC(═O)N5CCN(CC═3OC(C═2SC═C(C(═O)NC(C)C)N═2)═C(N═3)C4═CC═C(F)C═C4)CC5 |
| Example 373 | C═5C═CC(═C(C═5C(═O)N4CCN(CC═2OC(C═1SC═C(C(═O)NC(C)C)N═1)═C(N═2)C3═CC═C(F)C═C3)CC4)C)C |
| Example 374 | CC1═C(C═C(C)C═C1)C(═O)N5CCN(CC═3OC(C═2SC═C(C(═O)NC(C)C)N═2)═C(N═3)C4═CC═C(F)C═C4)CC5 |
| Example 375 | CC1═CC(═CC(C)═C1)C(═O)N5CCN(CC═3OC(C═2SC═C(C(═O)NC(C)C)N═2)═C(N═3)C4═CC═C(F)C═C4)CC5 |
| Example 376 | CC═5C(═CC═C(C(═O)N4CCN(CC═2OC(C═1SC═C(C(═O)NC(C)C)N═1)═C(N═2)C3═CC═C(F)C═C3)CC4)C═5)C |
| Example 377 | C═1C═C(C═CC═1CC)C(═O)N5CCN(CC═3OC(C═2SC═C(C(═O)NC(C)C)N═2)═C(N═3)C4═CC═C(F)C═C4)CC5 |
| Example 378 | COC5═CC═C(C(═O)N4 CCN(CC═2OC(C═1SC═C(C(═O)NC(C)C)N═1)═C(N═2)C3═CC═C(F)C═C3)CC4)C═C5 |
| Example 379 | CC1═CC═CC(═C1O)C(═O)N5CCN(CC═3OC(C═2SC═C(C(═O)NC(C)C)N═2)═C(N═3)C4═CC═C(F)C═C4)CC5 |
| Example 380 | OC═1C═CC═CC═1CC(═O)N5CCN(CC═3OC(C═2SC═C(C(═O)NC(C)C)N═2)═C(N═3)C4═CC═C(F)C═C4)CC5 |
| Example 381 | C1═CC═C(C═C1O)CC(═O)N5CCN(CC═3OC(C═2SC═C(C(═O)NC(C)C)N═2)═C(N═3)C4═CC═C(F)C═C4)CC5 |
| Example 382 | C1═CC(═C(C═C1C)C(═O)N5CCN(CC═3OC(C═2SC═C(C(═O)NC(C)C)N═2)═C(N═3)C4═CC═C(F)C═C4)CC5)O |
| Example 383 | OC1═CC═C(O)C═C1C(═O)N5CCN(CC═3OC(C═2SC═C(C(═O)NC(C)C)N═2)═C(N═3)C4═CC═C(F)C═C4)CC5 |
| Example 384 | OC1═C(O)C═CC═C1C(═O)N5CCN(CC═3OC(C═2SC═C(C(═O)NC(C)C)N═2)═C(N═3)C4═CC═C(F)C═C4)CC5 |
| Example 385 | OC═5C(C(═O)N4CCN(CC═2OC(C═1SC═C(C(═O)NC(C)C)N═1)═C(N═2)C3═CC═C(F)C═C3)CC4)═CC═C(C═5)O |
| Example 386 | FC═1C(C)═CC═C(C═1)C(═O)N5CCN(CC═3OC(C═2SC═C(C(═O)NC(C)C)N═2)═C(N═3)C4═CC═C(F)C═C4)CC5 |
| Example 387 | CC═5C(C(═O)N4CCN(CC═2OC(C═1SC═C(C(═O)NC(C)C)N═1)═C(N═2)C3═CC═C(F)C═C3)CC4)═CC(═CC═5)F |
| Example 388 | ClC1═CC═CC═C1C(═O)N5CCN(CC═3OC(C═2SC═C(C(═O)NC(C)C)N═2)═C(N═3)C4═CC═C(F)C═C4)CC5 |
| Example 389 | O═C(C1═CN═C(Cl)C═C1)N5CCN(CC═3OC(C═2SC═C(C(═O)NC(C)C)N═2)═C(N═3)C4═CC═C(F)C═C4)CC5 |
| Example 390 | ClC═1N═CC═CC═1C(═O)N5CCN(CC═3OC(C═2SC═C(C(═O)NC(C)C)N═2)═C(N═3)C4═CC═C(F)C═C4)CC5 |
| Example 391 | ClC1═NC═CC(═C1)C(═O)N5CCN(CC═3OC(C═2SC═C(C(═O)NC(C)C)N═2)═C(N═3)C4═CC═C(F)C═C4)CC5 |
| Example 392 | O═C(C1═NC═CC(═C1)Cl)N5CCN(CC═3OC(C═2SC═C(C(═O)NC(C)C)N═2)═C(N═3)C4═CC═C(F)C═C4)CC5 |
| Example 393 | O═C(C1═C(C═NC═C1)Cl)N5CCN(CC═3OC(C═2SC═C(C(═O)NC(C)C)N═2)═C(N═3)C4═CC═C(F)C═C4)CC5 |
| Example 394 | FC═1C═CC═C(F)C═1C(═O)N5CCN(CC═3OC(C═2SC═C(C(═O)NC(C)C)N═2)═C(N═3)C4═CC═C(F)C═C4)CC5 |
| Example 395 | O═C(C1═CC═CC(F)═C1F)N5CCN(CC═3OC(C═2SC═C(C(═O)NC(C)C)N═2)═C(N═3)C4═CC═C(F)C═C4)CC5 |
| Example 396 | O═C(C1═CC═C(F)C═C1F)N5CCN(CC═3OC(C═2SC═C(C(═O)NC(C)C)N═2)═C(N═3)C4═CC═C(F)C═C4)CC5 |
| Example 397 | FC═1C═C(F)C═C(C═1)C(═O)N5CCN(CC═3OC(C═2SC═C(C(═O)NC(C)C)N═2)═C(N═3)C4═CC═C(F)C═C4)CC5 |
| Example 398 | O═C(C1═CC═C(F)C(═C1)F)N5CCN(CC═3OC(C═2SC═C(C(═O)NC(C)C)N═2)═C(N═3)C4═CC═C(F)C═C4)CC5 |
| Example 399 | O═C(CC(CC(C)(C)C)C)N4CCN(CC═2OC(C═1SC═C(C(═O)NC(C)C)N═1)═C(N═2)C3═CC═C(F)C═C3)CC4) |
| Example 400 | C═5C6═CC═CC═C6OC═5C(═O)N4CCN(CC═2OC(C═1SC═C(C(═O)NC(C)C)N═1)═C(N═2)C3═CC═C(F)C═C3)CC4 |
| Example 401 | O═C(C2CCC1═CC═CC═C12)N6CCN(CC═4OC(C═3SC═C(C(═O)NC(C)C)N═3)═C(N═4)C5═CC═C(F)C═C5)CC6 |
| Example 402 | ClC(C(═O)N4CCN(CC═2OC(C═1SC═C(C(═O)NC(C)C)N═1)═C(N═2)C3═CC═C(F)C═C3)CC4)(Cl)Cl |
| Example 403 | O═C(/C═C/C═1C(O)═CC═CC═1)N5CCN(CC═3OC(C═2SC═C(C(═O)NC(C)C)N═2)═C(N═3)C4═CC═C(F)C═C4)CC5 |
| Example 404 | O═C(C1═CC═C(C(C)═O)C═C1)N5CCN(CC═3OC(C═2SC═C(C(═O)NC(C)C)N═2)═C(N═3)C4═CC═C(F)C═C4)CC5 |
| Example 405 | O═C(/C═C/C1═CC(O)═CC═C1)N5CCN(CC═3OC(C═2SC═C(C(═O)NC(C)C)N═2)═C(N═3)C4═CC═C(F)C═C4)CC5 |
| Example 406 | CC(═O)C1═CC═CC(═C1)C(═O)N5CCN(CC═3OC(C═2SC═C(C(═O)NC(C)C)N═2)═C(N═3)C4═CC═C(F)C═C4)CC5 |
| Example 407 | CC(═O)C1═CC═CC═C1 C(═O)N5CCN(CC═3OC(C═2SC═C(C(═O)NC(C)C)N═2)═C(N═3)C4═CC═C(F)C═C4)CC5 |
| Example 408 | C5═CC(═CC═C5C(═O)N4CCN(CC═2OC(C═1SC═C(C(═O)NC(C)C)N═1)═C(N═2)C3═CC═C(F)C═C3)CC4)C(C)C |
| Example 409 | CC═1C═CC═CC═1CCC(═O)N5CCN(CC═3OC(C═2SC═C(C(═O)NC(C)C)N═2)═C(N═3)C4═CC═C(F)C═C4)CC5 |
| Example 410 | O═C(CCC═1C═C(C)C═CC═1)N5CCN(CC═3OC(C═2SC═C(C(═O)NC(C)C)N═2)═C(N═3)C4═CC═C(F)C═C4)CC5 |
| Example 411 | O═C(C(CC)C1═CC═CC═C1)N5CCN(CC═3OC(C═2SC═C(C(═O)NC(C)C)N═2)═C(N═3)C4═CC═C(F)C═C4)CC5 |
| Example 412 | CC═1C═C(C)C═C(C═1C(═O)N5CCN(CC═3OC(C═2SC═C(C(═O)NC(C)C)N═2)═C(N═3)C4═CC═C(F)C═C4)CC5)C |
| Example 413 | C1═CC(═CC═C1CCC)C(═O)N5CCN(CC═3OC(C═2SC═C(C(═O)NC(C)C)N═2)═C(N═3)C4═CC═C(F)C═C4)CC5 |
| Example 414 | O═C(C═1C═CC═2OCOC═2C═1)N6CCN(CC═4OC(C═3SC═C(C(═O)NC(C)C)N═3)═C(N═4)C5═CC═C(F)C═C5)CC6 |
| Example 415 | O═C(/C═C/C═1C(F)═CC═CC═1)N5CCN(CC═3OC(C═2SC═C(C(═O)NC(C)C)N═2)═C(N═3)C4═CC═C(F)C═C4)CC5 |
| Example 416 | C═1C═CC═C(C═1OCC)C(═O)N5CCN(CC═3OC(C═2SC═C(C(═O)NC(C)C)N═2)═C(N═3)C4═CC═C(F)C═C4)CC5 |
| Example 417 | CC═1C═CC═CC═1OCC(═O)N5CCN(CC═3OC(C═2SC═C(C(═O)NC(C)C)N═2)═C(N═3)C4═CC═C(F)C═C4)CC5 |
| Example 418 | O═C(CCC1═CC═CC═C1O)N5CCN(CC═3OC(C═2SC═C(C(═O)NC(C)C)N═2)═C(N═3)C4═CC═C(F)C═C4)CC5 |
| Example 419 | O═C(CC1═C(OC)C═CC═C1)N5CCN(CC═3OC(C═2SC═C(C(═O)NC(C)C)N═2)═C(N═3)C4═CC═C(F)C═C4)CC5 |
| Example 420 | O═C(C(C═1C═CC(O)═CC═1)C)N5CCN(CC═3OC(C═2SC═C(C(═O)NC(C)C)N═2)═C(N═3)C4═CC═C(F)C═C4)CC5 |
| Example 421 | O═[N+]([O−])C1═CC═CC═C1C(═O)N5CCN(CC═3OC(C═2SC═C(C(═O)NC(C)C)N═2)═C(N═3)C4═CC═C(F)C═C4)CC5 |
| Example 422 | O═[N+]([O−])C═1C═CC═C(C═1)C(═O)N5CCN(CC═3OC(C═2SC═C(C(═O)NC(C)C)N═2)═C(N═3)C4═CC═C(F)C═C4)CC5 |
| Example 423 | COC1═C(C(O)═CC═C1)C(═O)N5CCN(CC═3OC(C═2SC═C(C(═O)NC(C)C)N═2)═C(N═3)C4═CC═C(F)C═C4)CC5 |
| Example 424 | OC1═C(C═C(OC)C═C1)C(═O)N5CCN(CC═3OC(C═2SC═C(C(═O)NC(C)C)N═2)═C(N═3)C4═CC═C(F)C═C4)CC5 |
| Example 425 | OC═1C═C(OC)C═CC═1 C(═O)N5CCN(CC═3OC(C═2SC═C(C(═O)NC(C)C)N═2)═C(N═3)C4═CC═C(F)C═C4)CC5 |
| Example 426 | O═C(C1═CC)(O)═C(OC)C═C1)N5CCN(CC═3OC(C═2SC═C(C(═O)NC(C)C)N═2)═C(N═3)C4═CC═C(F)C═C4)CC5 |
| Example 427 | OC═1C(O)═C(C═C(C═1)C(═O)N5CCN(CC═3OC(C═2SC═C(C(═O)NC(C)C)N═2)═C(N═3)C4═CC═C(F)C═C4)CC5)O |
| Example 428 | FC5═CC(CC(═O)N4CC N(CC═2OC(C═1SC═C(C(═O)NC(C)C)N═1)═C(N═2)C3═CC═C(F)C═C3)CC4)═CC═C5O |
| Example 429 | O═C(C═1C═C(N═C(C═1)Cl)C)N5CCN(CC═3OC(C═2SC═C(C(═O)NC(C)C)N═2)═C(N═3)C4═CC═C(F)C═C═C4)CC5 |
| Example 430 | FC═1C═CC═C(C═1CC(═O)N5CCN(CC═30C(C═2SC═C(C(═O)NC(C)C)N═2)═C(N═3)C4═CC═C(F)C═C═C4)CC5)F |
| Example 431 | O═C(C2═CC═CC1═CC═CC)═C12)N6CCN(CC═4OC(C═3SC═C(C(═O)NC(C)C)N═3)═C(N═4)C5═CC═C(F)C═C5)CC6) |
| Example 432 | O═C(C1═CC═C(Cl)C═C1O)N5CCN(CC═3OC(C═2SC═C(C(═O)NC(C)C)N═2)═C(N═3)C4═CC═C(F)C═C4)CC5 |
| Example 433 | OC═5C(C(═O)N4CCN(CC═2OC(C═1SC═C(C(═O)NC(C)C)N═1)═C(N═2)C3═CC═C(F)C═C3)CC4)═CC(═CC═5)Cl |
| Example 434 | O═C(C1═CC═2C═CC═NC═2C═C1)N6CCN(CC═4OC(C═3SC═C(C(═O)NC(C)C)N═3)═C(N═4)C5═CC═C(F)C═C5)CC6 |
| Example 435 | OC5═NC═C(C(═O)N4CCN(CC═2OC(C═1SC═C(C(═O)NC(C)C)N═1)═C(N═2)C3═CC═C(F)C═C3)CC4)C═C5Cl |
| Example 436 | O═C(C1═CC═C(F)C═C1Cl)N5CCN(CC═3OC(C═2SC═C(C(═O)NC(C)C)N═2)═C(N═3)C4═CC═C(F)C═C4)CC5 |
| Example 437 | FC═1C═CC═C(C═1C(═O)N5CCN(CC═3OC(C═2SC═C(C(═O)NC(C)C)N═2)═C(N═3)C4═CC═C(F)C═C4)CC5)Cl |
| Example 438 | CC(C)(OC(NCC(═O)N4CCN(CC═2OC(C═1SC═C(C(═O)NC(C)C)N═1)═C(N═2)C3═CC═C(F)C═C3)CC4)═O)C |
| Example 439 | O═C(C═1OC2═CC═CC═C2C═1C)N6CCN(CC═4OC(C═3SC═C(C(═O)NC(C)C)N═3)═C(N═4)C5═CC═C(F)C═C5)CC6 |
| Example 440 | C═1C2═C(C═CC═1)CC(C2)CC(═O)N6CCN(CC═4OC(C═3SC═C(C(═O)NC(C)C)N═3)═C(N═4)C5═CC═C(F)C═C5)CC6 |
| Example 441 | C5CC6═C(CC5C(═O)N4CCN(CC═2OC(C═1SC═C(C(═O)NC(C)C)N═1)═C(N═2)C3═CC═C(F)C═C3)CC4)C═CC═C6 |
| Example 442 | O═C(/C═C/C═1C═CC═CC═1OC)N5CCN(CC═3OC(C═2SC═C(C(═O)NC(C)C)N═2)═C(N═3)C4═CC═C(F)C═C4)CC5 |
| Example 443 | O═C(C1═CSC2═C1C═CC═C2)N6CCN(CC═4OC(C═3SC═C(C(═O)NC(C)C)N═3)═C(N═4)C5═CC═C(F)C═C5)CC6 |
| Example 444 | C═1C═C2C═C(SC2═CC═1)C(═O)N6CCN(CC═4OC(C═3SC═C(C(═O)NC(C)C)N═3)═C(N═4)C5═CC═C(F)C═C5)CC6 |
| Example 445 | O═C(C1═CC═C(CCCC)C═C1)N5CCN(CC═3OC(C═2SC═C(C(═O)NC(C)C)N═2)═C(N═3)C4═CC═C(F)C═C4)CC5 |
| Example 446 | O═C(C1═CC═C(C(C)(C)C)C═C1)N5CCN(CC═3OC(C═2SC═C(C(═O)NC(C)C)N═2)═C(N═3)C4═CC═C(F)C═C4)CC5 |
| Example 447 | O═C(C1═CC═C(N(C═O)C)C═C1)N5CCN(CC═3OC(C═2SC═C(C(═O)NC(C)C)N═2)═C(N═3)C4═CC═C(F)C═C4)CC5 |
| Example 448 | O═C(C═1C═C(NC(C)═O)C═CC═1)N5CCN(CC═3OC(C═2SC═C(C(═O)NC(C)C)N═2)═C(N═3)C4═CC═C(F)C═C4)CC5 |
| Example 449 | O═C(COC═1C(C═O)═CC═CC═1)N5CCN(CC═3OC(C═2SC═C(C(═O)NC(C)C)N═2)═C(N═3)C4═CC═C(F)C═C4)CC5 |
| Example 450 | CC(═O)OC1═CC═C(C═C1)C(═O)N5CCN(CC═3OC(C═2SC═C(C(═O)NC(C)C)N═2)═C(N═3)C4═CC═C(F)C═C4)CC5 |
| Example 451 | O═C(OC)C5═CC═C(C(═O)N4CCN(CC═2OC(C═1SC═C(C(═O)NC(C)C)N═1)═C(N═2)C3═CC═C(F)C═C3)CC4)C═C5 |
| Example 452 | C5═C(C═CC═C5C(═O)N4CCN(CC═2OC(C═1SC═C(C(═O)NC(C)C)N═1)═C(N═2)C3═CC═C(F)C═C3)CC4)OC(═O)C |
| Example 453 | CC(═O)OC1═CC═CC═C1C(═O)N5CCN(CC═3OC(C═2SC═C(C(═O)NC(C)C)N═2)═C(N═3)C4═CC═C(F)C═C4)CC5 |
| Example 454 | O1C═2C═CC(═CC═2OC1)CC(═O)N6CCN(CC═4OC(C═3SC═C(C(═O)NC(C)C)N═3)═C(N═4)C5═CC═C(F)C═C5)CC6 |
| Example 455 | C1═CC(═CC═C1OCCC)C(═O)N5CCN(CC═3OC(C═2SC═C(C(═O)NC(C)C)N═2)═C(N═3)C4═CC═C(F)C═C4)CC5 |
| Example 456 | O═C(C1═CC═C(OC(C)C)C═C1)N5CCN(CC═3OC(C═2SC═C(C(═O)NC(C)C)N═2)═C(N═3)C4═CC═C(F)C═C4)CC5 |
| Example 457 | [O−][N+](═O)C1═C(C(═CC═C1)C)C(═O)N5CCN(CC═3OC(C═2SC═C(C(═O)NC(C)C)N═2)═C(N═3)C4═CC═C(F)C═C4)CC5 |
| Example 458 | O═[N+]([O−])C1═C(C═CC═C1C(═O)N5CCN(CC═3OC(C═2SC═C(C(═O)NC(C)C)N═2)═C(N═3)C4═CC═C(F)C═C4)CC5)C |
| Example 459 | [O−][N+](═O)C1═CC═C(C═C1C(═O)N5CCN(CC═3OC(C═2SC═C(C(═O)NC(C)C)N═2)═C(N═3)C4═CC═C(F)C═C4)CC5)C |
| Example 460 | CC═5C(C(═O)N4CCN(CC═2OC(C═1SC═C(C(═O)NC(C)C)N═1)═C(N═2)C3═CC═C(F)C═C3)CC4)═CC═CC═5[N+]([O−])═O |
| Example 461 | C1═CC(═C(C═C1OC)C(═O)N5CCN(CC═3OC(C═2SC═C(C(═O)NC(C)C)N═2)═C(N═3)C4═CC═C(F)C═C4)CC5)OC |
| Example 462 | COC1═CC(OC)═CC═C1C(═O)N5CCN(CC═3OC(C═2SC═C(C(═O)NC(C)C)N═2)═C(N═3)C4═CC═C(F)C═C4)CC5 |
| Example 463 | COC═1C(═C(OC)C═CC═1)C(═O)N5CCN(CC═3OC(C═2SC═C(C(═O)NC(C)C)N═2)═C(N═3)C4═CC═C(F)C═C4)CC5 |
| Example 464 | C═1C(═CC(═CC═1OC)C(═O)N5CCN(CC═3OC(C═2SC═C(C(═O)NC(C)C)N═2)═C(N═3)C4═CC═C(F)C═C4)CC5)OC |
| Example 465 | C═1C═CC(═C(C═1OC)OC)C(═O)N5CCN(CC═3OC(C═2SC═C(C(═O)NC(C)C)N═2)═C(N═3)C4═CC═C(F)C═C4)CC5 |
| Example 466 | CC5═CC═C(SCC(═O)N4CCN(CC═2OC(C═1SC═C(C(═O)NC(C)C)N═1)═C(N═2)C3═CC═C(F)C═C3)CC4)C═C5 |
| Example 467 | O═C(/C═C/C═1C═CC(Cl)═CC═1)N5CCN(CC═3OC(C═2SC═C(C(═O)NC(C)C)N═2)═C(N═3)C4═CC═C(F)C═C4)CC5 |
| Example 468 | O═C(/C═C/C═1C(Cl)═CC═CC═1)N5CCN(CC═3OC(C═2SC═C(C(═O)NC(C)C)N═2)═C(N═3)C4═CC═C(F)C═C4)CC5 |
| Example 469 | [O−][N+](C1═CC═C(C═C1O)C(═O)N5CCN(CC═3OC(C═2SC═C(C(═O)NC(C)C)N═2)═C(N═3)C4═CC═C(F)C═C4)CC5)═O |
| Example 470 | O═C(C1═CC(O)═CC═C1 [N+]([O−])═O)N5CCN(CC═3OC(C═2SC═C(C(═O)NC(C)C)N═2)═C(N═3)C4═CC═C(F)C═C4)CC5 |
| Example 471 | OC5═CC═C(C═C5C(═O)N4CCN(CC═2OC(C═1SC═C(C(═O)NC(C)C)N═1)═C(N═2)C3═CC═C(F)C═C3)CC4)[N+](═O)[O−] |
| Example 472 | O═[N+]([O−])C1═CC═C(C(═C1)C(═O)N5CCN(CC═3OC(C═2SC═C(C(═O)NC(C)C)N═2)═C(N═3)C4═CC═C(F)C═C4)CC5)F |
| Example 473 | O═[N+]([O−])C1═CC═C(C═C1C(═O)N5CCN(CC═3OC(C═2SC═C(C(═O)NC(C)C)N═2)═C(N═3)C4═CC═C(F)C═C4)CC5)F |
| Example 474 | O═[N+]([O−])C═1C(═CC═C(C═1)C(═O)N5CCN(CC═3OC(C═2SC═C(C(═O)NC(C)C)N═2)═C(N═3)C4═CC═C(F)C═C4)CC5)F |
| Example 475 | COC1═CC═C(Cl)C═C1C(═O)N5CCN(CC═3OC(C═2SC═C(C(═O)NC(C)C)N═2)═C(N═3)C4═CC═C(F)C═C4)CC5 |
| Example 476 | ClC═1C═C(OC)C(═CC═1)C(═O)N5CCN(CC═3OC(C═2SC═C(C(═O)NC(C)C)N═2)═C(N═3)C4═CC═C(F)C═C4)CC5 |
| Example 477 | O═C(CC1═CC(═C(O)C═C1)Cl)N5CCN(CC═3OC(C═2SC═C(C(═O)NC(C)C)N═2)═C(N═3)C4═CC═C(F)C═C4)CC5 |
| Example 478 | O═C(C═1C═C(N═C(C═1)Cl)OC)N5CCN(CC═3OC(C═2SC═C(C(═O)NC(C)C)N═2)═C(N═3)C4═CC═C(F)C═C4)CC5 |
| Example 479 | OC6═CC═C1C(C═CC(═C1)C(═O)N5CCN(CC═3OC(C═2SC═C(C(═O)NC(C)C)N═2)═C(N═3)C4═CC═C(F)C═C4)CC5)═C6 |
| Example 480 | O═C(C═1C2═CC═CC═C2C═CC═1O)N6CCN(CC═4OC(C═3SC═C(C(═O)NC(C)C)N═3)═C(N═4)C5═CC═C(F)C═C5)CC6 |
| Example 481 | O═C(C1═CC2═CC═CC═C2C═C1O)N6CCN(CC═4OC(C═3SC═C(C(═O)NC(C)C)N═3)═C(N═4)C5═CC═C(F)C═C5)CC6 |
| Example 482 | C1═CC═2C(C═C1)═CC═C(C═2O)C(═O)N6CCN(CC═4OC(C═3SC═C(C(═O)NC(C)C)N═3)═C(N═4)C5═CC═C(F)C═C5)CC6 |
| Example 483 | OC1═CC═CC2═CC═C(N═C12)C(═O)N6CCN(CC═4OC(C═3SC═C(C(═O)NC(C)C)N═3)═C(N═4)C5═CC═C(F)C═C5)CC6 |
| Example 484 | CC(C)(OC(NC(C(═O)N4CCN(CC═2OC(C═1SC═C(C(═O)NC(C)C)N═1)═C(N═2)C3═CC═C(F)C═C3)CC4)C)═O)C |
| Example 485 | CC(C)(OC(NC(C(═O)N4CCN(CC═2OC(C═1SC═C(C(═O)NC(C)C)N═1)═C(N═2)C3═CC═C(F)C═C3)CC4)C)═O)C |
| Example 486 | O═C(C═1C(═CC═CC═1)C(F)(F)F)N5CCN(CC═3OC(C═2SC═C(C(═O)NC(C)C)N═2)═C(N═3)C4═CC═C(F)C═C4)CC5 |
| Example 487 | [H]N2C1═CC═CC═C1N═C2CCC(═O)N6CCN(CC═4OC(C═3SC═C(C(═O)NC(C)C)N═3)═C(N═4)C5═CC═C(F)C═C5)CC6 |
| Example 488 | ClC1═C(Cl)C═CC═C1C(═O)N5CCN(CC═3OC(C═2SC═C(C(═O)NC(C)C)N═2)═C(N═3)C4═CC═C(F)C═C4)CC5 |
| Example 489 | ClC1═CC(Cl)═CC═C1C(═O)N5CCN(CC═3OC(C═2SC═C(C(═O)NC(C)C)N═2)═C(N═3)C4═CC═C(F)C═C4)CC5 |
| Example 490 | ClC═5C(C(═O)N4CCN(CC═2OC(C═1SC═C(C(═O)NC(C)C)N═1)═C(N═2)C3═CC═C(F)C═C3)CC4)═CC(═CC═5)Cl |
| Example 491 | O═C(C1═C(C(═C(C(═C1)F)F)O)F)N5CCN(CC═3OC(C═2SC═C(C(═O)NC(C)C)N═2)═C(N═3)C4═CC═C(F)C═C4)CC5 |
| Example 492 | O1C═2C═CC(═CC═2OC1)/ C═C/C(═O)N6CCN(CC═4OC(C═3SC═C(C(═O)NC(C)C)N═3)═C(N═4)C5═CC═C(F)C═C5)CC6 |
| Example 493 | C1═CC(═CC═C1C)C(CCC(═O)N5CCN(CC═3OC(C═2SC═C(C(═O)NC(C)C)N═2)═C(N═3)C4═CC═C(F)C═C4)CC5)═O |
| Example 494 | O═C(C1═CC═C(CCCCC)C═C1)N5CCN(CC═3OC(C═2SC═C(C(═O)NC(C)C)N═2)═C(N═3)C4═CC═C(F)C═C4)CC5 |
| Example 495 | O═C(C1═CC(═C(F)C═C1Cl)F)N5CCN(CC═3OC(C═2SC═C(C(═O)NC(C)C)N═2)═C(N═3)C4═CC═C(F)C═C4)CC5 |
| Example 496 | FC1═CC(Cl)═C(C═C1C(═O)N5CCN(CC═3OC(C═2SC═C(C(═O)NC(C)C)N═2)═C(N═3)C4═CC═C(F)C═C4)CC5)F |
| Example 497 | O═C(C1═CC═C(N(CC)CC)C═C1)N5CCN(CC═3OC(C═2SC═C(C(═O)NC(C)C)N═2)═C(N═3)C4═CC═C(F)C═C4)CC5 |
| Example 498 | COC5═CC(/C═C/C(═O)N4CCN(CC═2OC(C═1SC═C(C(═O)NC(C)C)N═1)═C(N═2)C3═CC═C(F)C═C3)CC4)═CC═C5O |
| Example 499 | O═C(/C═C/C1═CC(O)═C(OC)C═C1)N5CCN(CC═3OC(C═2SC═C(C(═O)NC(C)C)N═2)═C(N═3)C4═CC═C(F)C═C4)CC5 |
| Example 500 | O═C(C1═CC═C(OCCCC)C═C1)N5CCN(CC═3OC(C═2SC═C(C(═O)NC(C)C)N═C)═C(N═3)C4═CC═C(F)C═C4)CC5 |
| Example 501 | O═C(C═2OC1═C(C═C(Cl)C═C1)C═2)N6CCN(CC═4OC(C═3SC═C(C(═O)NC(C)C)N═3)═C(N═4)C5═CC═C(F)C═C5)CC6 |
| Example 502 | C1═CC(═C(C═C1OC)C(═O)N5CCN(CC═3OC(C═2SC═C(C(═O)NC(C)C)N═2)═C(N═3)C4═CC═C(F)C═C4)CC5)[N+]([O−])═O |
| Example 503 | O═C(C1═CC(OC)═C(O)C(OC)═C1)N5CCN(CC═3OC(C═2SC═C(C(═O)NC(C)C)N═2)═C(N═3)C4═CC═C(F)C═C4)CC5 |
| Example 504 | C6═CC═CC═C6C═5C(C(═O)N4CCN(CC═2OC(C═1SC═C(C(═O)NC(C)C)N═1)═C(N═2)C3═CC═C(F)C═C3)CC4)═CC═CC═5 |
| Example 505 | BrC1═CC═CC(═C1)C(═O)N5CCN(CC═3OC(C═2SC═C(C(═O)NC(C)C)N═2)═C(N═3)C4═CC═C(F)C═C4)CC5 |
| Example 506 | C═1C═C(C═CC═1Br)C(═O)N5CCN(CC═3OC(C═2SC═C(C(═O)NC(C)C)N═2)═C(N═3)C4═CC═C(F)C═C4)CC5 |
| Example 507 | [O−][N+](═O)C1═C(C═CC═C1C(═O)N5CCN(CC═3OC(C═2SC═C(C(═O)NC(C)C)N═2)═C(N═3)C4═CC═C(F)C═C4)CC5)Cl |
| Example 508 | O═[N+]([O−])C1═CC═C(C(N═C1)C(═O)N5CCN(CC═3OC(C═2SC═C(C(═O)NC(C)C)N═2)═C(N═3)C4═CC═C(F)C═C4)CC5)Cl |
| Example 509 | O═C(C1═C(C═C([N+]([O−])═O)C═C1)Cl)N5CCN(CC═3OC(C═2SC═C(C(═O)NC(C)C)N═2)═C(N═3)C4═CC═C(F)C═C4)CC5 |
| Example 510 | O═[N+]([O−])C1═CC(═CC═C1C(═O)N5CCN(CC═3OC(C═2SC═C(C(═O)NC(C)C)N═2)═C(N═3)C4═CC═C(F)C═C4)CC5)Cl |
| Example 511 | O═[N+]([O−])C1═CC(═CC═C1Cl)C(═C)N5CCN(CC═3OC(C═2SC═C(C(═O)NC(C)C)N═2)═C(N═3)C4═CC═C(F)C═C4)CC5 |
| Example 512 | O═C(C1═CC(Cl)═CC═C1 [N+](═O([O−])N5CCN(CC═3OC(C═2SC═C(C(═O)NC(C)C)N═2)═C(N═3)C4═CC═C(F)C═C4)CC5 |
| Example 513 | C═5C═CC(═NC═5C(═O)N4CCN(CC═2OC(C═1SC═C(C(═O)NC(C)C)N═1)═C(N═2)C3═CC═C(F)C═C3)CC4)BR |
| Example 514 | O═C(C═1C═NC═C(Br)C═1)N5CCN(CC═3OC(C═2SC═C(C(═O)NC(C)C)N═2)═C(N═3)C4═CC═C(F)C═C4)CC5 |
| Example 515 | [O−][N+](═O)C═5C(C(═O)N4CCN(CC═2OC(C═1SC═C(C(═O)NC(C)C)N═1)═C(N═2)C3═CC═C(F)C═C3)CC4)═CC(F)═C(F)C═5 |
| Example 516 | FC(F)(C═1C═CC(═CC═1)CC(═O)N5CCN(CC═3OC(C═2SC═C(C(═O)NC(C)C)N═2)═C(N═3)C4═CC═C(F)C═C4)CC5)F |
| Example 517 | CC(C)CC5═CC═C(C(C(═O)N4CCN(CC═2OC(C═1SC═C(C(═O)NC(C)C)N═1)═C(N═2)C3═CC═C(F)C═C3)CC4)C)C═C5 |
| Example 518 | ClC1═C(O)C(═CC(═C1)C(═O)N5CCN(CC═3OC(C═2SC═C(C(═O)NC(C)C)N═2)═C(N═3)C4═CC═C(F)C═C4)CC5)Cl |
| Example 519 | FC═1C(F)═C(C(═C(C═1 F)C(═O)N5CCN(CC═3OC(C═2SC═C(C(═O)NC(C)C)N═2)═C(N═3)C4═CC═C(F)C═C4)CC5)F)F |
| Example 520 | O═C(C1═C(OC)C═C(C═C1OC)OC)N5CCN(CC═3OC(C═2SC═C(C(═O)NC(C)C)N═2)═C(N═3)C4═CC═C(F)C═C4)CC5 |
| Example 521 | COC1═CC(═C(C═C1OC)OC)C(═O)N5CCN(CC═3OC(C═2SC═C(C(═O)NC(C)C)N═2)═C(N═3)C4═CC═C(F)C═C4)CC5 |
| Example 522 | COC1═C(OC)C(═CC(═C1)C(═O)N5CCN(CC═3OC(C═2SC═C(C(═O)NC(C)C)N═2)═C(N═3)C4═CC═C(F)C═C4)CC5)OC |
| Example 523 | COC1═C(OC)C═CC(═C1OC)(═O)N5CCN(CC═3OC(C═2SC═C(C(═O)NC(C)C)N═2)═C(N═3)C4═CC═C(F)C═C4)CC5 |
| Example 524 | C═1C═C(C═CC═1)CC6═C(C(═O)N5CCN(CC═3OC(C═2SC═C(C(═O)NC(C)C)N═2)═C(N═3)C4═CC═C(F)C═C4)CC5)C═CC═C6 |
| Example 525 | O═C(C2═CC═CC(OC1═CC═CC═C1)═C2)N6CCN(CC═4OC(C═3SC═C(C(═O)NC(C)C)N═3)═C(N═4)C5═CC═C(F)C═C5)CC6 |
| Example 526 | C1═CC(═CC═C1)OC6═C(C(═O)N5CCN(CC═3OC(C═2SC═C(C(═O)NC(C)C)N═2)═C(N═3)C4═CC═C(F)C═C4)CC5)C═CC═C6 |
| Example 527 | O═C(C2═CC═C(OC1═CC═CC═C1)C═C2)N6CCN(CC═4OC(C═3SC═C(C(═O)NC(C)C)N═3)═C(N═4)C5═CC═C(F)C═C5)CC6 |
| Example 528 | C1═CC(═CC═C1O)C2═CC═C(C═C2)C(═O)N6CCN(CC═4OC(C═3SC═C(C(═O)NC(C)C)N═3)═C(N═4)C5═CC═C(F)C═C5)CC6 |
| Example 529 | BrC═1C═CC(═CC═1C)C(═O)N5CCN(CC═3OC(C═2SC═C(C(═O)NC(C)C)N═2)═C(N═3)C4═CC═C(F)C═C4)CC5 |
| Example 530 | BrC1═C(C)C═CC(═C1)C(═O)N5CCN(CC═3OC(C═2SC═C(C(═O)NC(C)C)N═2)═C(N═3)C4═CC═C(F)C═C4)CC5 |
| Example 531 | O═C(CC1═CC═C(Br)C═C1)N5CCN(CC═3OC(C═2SC═C(C(═O)NC(C)C)N═2)═C(N═3)C4═CC═C(F)C═C4)CC5 |
| Example 532 | CC(OC(═O)N1C(CCC1)C(═O)N5CCN(CC═3OC(C═2SC═C(C(═O)NC(C)C)N═2)═C(N═3)C4═CC═C(F)C═C4)CC5)(C)C |
| Example 533 | O═C(/C═C/C1═C(C(F)(F)F)C═CC═C1)N5CCN(CC═3OC(C═2SC═C(C(═O)NC(C)C)N═2)═C(N═3)C4═CC═C(F)C═C4)CC5 |
| Example 534 | O═C(C1═C(OCC)C═CC2═C1C═CC═C2)N6CCN(CC═4OC(C═3SC═C(C(═O)NC(C)C)N═3)═C(N═4)C5═CC═C(F)C═C5)CC6 |
| Example 535 | CC(C)(OC(NC(C(═O)N4CCN(CC═2OC(C═1SC═C(C(═O)NC(C)C)N═1)═C(N═2)C3═CC═C(F)C═C3)CC4)C(C)C)═O)C |
| Example 536 | CC(C)(OC(NC(C(═O)N4CCN(CC═2OC(C═1SC═C(C(═O)NC(C)C)N═1)═C(N═2)C3═CC═C(F)C═C3)CC4)C(C)C)═O)C |
| Example 537 | O═C(CCC═1C═C(C═CC═1)C(F)(F)F)N5CCN(CC═3OC(C═2SC═C(C(═O)NC(C)C)N═2)═C(N═3)C4═CC═C(F)C═C4)CC5 |
| Example 538 | O═C(C═2C═CC(N1N═C(C)CC1═O)═CC═2)N6CCN(CC═4OC(C═3SC═C(C(═O)NC(C)C)N═3)═C(N═4)C5═CC═C(F)C═C5)CC6 |
| Example 539 | CC5═C(C(═O)N4CCN(CC═2OC(C═1SC═C(C(═O)NC(C)C)N═1)═C(N═2)C3═CC═C(F)C═C3)CC4)SC(═N5)Br |
| Example 540 | C1═CC2═C(C═C1)C═C7C(═C2C(═O)N6CCN(CC═4OC(C═3SC═C(C(═O)NC(C)C)N═3)═C(N═4)C5═CC═C(F)C═C5)CC6)C═CC═C7 |
| Example 541 | CC(C)C5═CC(C(═O)N4CCN(CC═2OC(C═1SC═C(C(═O)NC(C)C)N═1)═C(N═2)C3═CC═C(F)C═C3)CC4)═C(C(═C5)C(C)C)O |
| Example 542 | CC(═O)NC(C(═O)N4CCN(CC═2OC(C═1SC═C(C(═O)NC(C)C)N═1)═C(N═2)C3═CC═C(F)C═C3)CC4)CC5═CC═C(C═C5)O |
| Example 543 | ClC1═CC(Cl)═CC(═C1C(═O)N5CCN(CC═3OC(C═2SC═C(C(═O)NC(C)C)N═2)═C(N═3)C4═CC═C(F)C═C4)CC5)Cl |
| Example 544 | COC1═CC(═C(C═C1OC)[N+](═O)[O−])C(═O)N5CCN(CC═3OC(C═2SC═C(C(═O)NC(C)C)N═2)═C(N═3)C4═CC═C(F)C═C4)CC5 |
| Example 545 | O═C(C2═CC═C(OCC1═CC═CC═C1)C═C2)N6CCN(CC═4OC(C═3SC═C(C(═O)NC(C)C)N═3)═C(N═4)C5═CC═C(F)C═C5)CC6 |
| Example 546 | BrC═5C═CC═CC═5CCC(═O)N4CCN(CC═2OC(C═1SC═C(C(═O)NC(C)C)N═1)═C(N═2)C3═CC═C(F)C═C3)CC4 |
| Example 547 | O═C(CCC═1C═C(Br)C═CC═1)N5CCN(CC═3OC(C═2SC═C(C(═O)NC(C)C)N═2)═C(N═3)C4═CC═C(F)C═C4)CC5 |
| Example 548 | CC(OC(N5C(C(═O)N4CCN(CC═2OC(C═1SC═C(C(═O)NC(C)C)N═1)═C(N═2)C3═CC═C(F)C═C3)CC4)CCCC5)═O)(C)C |
| Example 549 | C6═CC═C(C═C6OC═1C═CC(═CC═1)C(═O)N5CCN(CC═3OC(C═2SC═C(C(═O)NC(C)C)N═2)═C(N═3)C4═CC═C(F)C═C4)CC5)O |
| Example 550 | CC(C(═O)N4CCN(CC═2OC(C═1SC═C(C(═O)NC(C)C)N═1)═C(N═2)C3═CC═C(F)C═C3)CC4)C═6C═CC5═CC(═CC═C5C═6)OC |
| Example 551 | CC(C)(OC(N1CC(NCC1)C(═O)N5CCN(CC═3OC(C═2SC═C(C(═O)NC(C)C)N═2)═C(N═3)C4═CC═C(F)C═C4)CC5)═O)C |
| Example 552 | CC(C)(OC(N1CC(NCC1)C(═O)N5CCN(CC═3OC(C═2SC═C(C(═O)NC(C)C)N═2)═C(N═3)C4═CC═C(F)C═C4)CC5)═O)C |
| Example 553 | COC1═CC(═C(C═C1)Br)C(═O)N5CCN(CC═3OC(C═2SC═C(C(═O)NC(C)C)N═2)═C(N═3)C4═CC═C(F)C═C4)CC5 |
| Example 554 | CC(OC(═O)N1CC(CC1C(═O)N5CCN(CC═3OC(C═2SC═C(C(═O)NC(C)C)N═2)═C(N═3)C4═CC═C(F)C═C4)CC5)O)(C)C |
| Example 555 | CC(C)C(C(═O)N4CCN(CC═2OC(C═1SC═C(C(═O)NC(C)C)N═1)═C(N═2)C3═CC═C(F)C═C3)CC4)N(C(OC(C)(C)C)═O)C |
| Example 556 | FC(F)(C5═CC([N+]([O−])═O)═C(C(═O)N4CCN(CC═2OC(C═1SC═C(C(═O)NC(C)C)N═1)═C(N═2)C3═CC═C(F)C═C3)CC4)C═C5)F |
| Example 557 | BrC1═CC═C(Cl)C(═C1)C(═O)N5CCN(CC═3OC(C═2SC═C(C(═O)NC(C)C)N═2)═C(N═3)C4═CC═C(F)C═C4)CC5 |
| Example 558 | O═C(C═1C═C(Br)C(═CC═1)Cl)N5CCN(CC═3OC(C═2SC═C(C(═O)NC(C)C)N═2)═C(N═3)C4═CC═C(F)C═C4)CC5 |
| Example 559 | C═1C═C(C═CC═1O)C(═O)C2═CC═CC═C2C(═O)N6CCN(CC═4OC(C═3SC═C(C(═O)NC(C)C)N═3)═C(N═4)C5═CC═C(F)C═C5)CC6 |
| Example 560 | C1═CC(═CC═C1F)C(═O)C6═C(C(═O)N5CCN(CC═3OC(C═2SC═C(C(═O)NC(C)C)N═2)═C(N═3)C4═CC═C(F)C═C4)CC5)C═CC═C6 |
| Example 561 | O═C(C(C2═CC(═C(C1═CC═CC═C1)C═C2)F)C)N6CCN(CC═4OC(C═3SC═C(C(═O)NC(C)C)N═3)═C(N═4)C5═CC═C(F)C═C5)CC6 |
| Example 562 | C═1C═C(C═CC═1l)C(═O)N5CCN(CC═3OC(C═2SC═C(C(═O)NC(C)C)N═2)═C(N═3)C4═CC═C(F)C═C4)CC5 |
| Example 563 | lC1═CC═CC(═C1)C(═O)N5CCN(CC═3OC(C═2SC═C(C(═O)NC(C)C)N═2)═C(N═3)C4═CC═C(F)C═C4)CC5 |
| Example 564 | O═C(OCC1═CC═CC═C1)N2C(CCC2)C(═O)N6CCN(CC═4OC(C═3SC═C(C(═O)NC(C)C)N═3)═C(N═4)C5═CC═C(F)C═C5)CC6 |
| Example 565 | CC(CC)C(C(═O)N4CCN(CC═2OC(C═1SC═C(C(═O)NC(C)C)N═1)═C(N═2)C3═CC═C(F)C═C3)CC4)NC(OC(C)(C)C)═O |
| Example 566 | CC(C)(OC(NC(C(═O)N4CCN(CC═2OC(C═1SC═C(C(═O)NC(C)C)N═1)═C(N═2)C3═CC═C(F)C═C3)CC4)CC(C)C)═O)C |
| Example 567 | O═C(C1═CC═CC═C1)C═2C═C(C═CC═2)C(C)C(═O)N6CCN(CC═4OC(C═3SC═C(C(═O)NC(C)C)N═3)═C(N═4)C5═CC═C(F)C═C5)CC6 |
| Example 568 | CC(C)(OC(NC(C(═O)N4CCN(CC═2OC(C═1SC═C(C(═O)NC(C)C)N═1)═C(N═2)C3═CC═C(F)C═C3)CC4)CC5═CC═CC═C5)═O)C |
| Example 569 | CC(C)(OC(NC(C(═O)N4CCN(CC═2OC(C═1SC═C(C(═O)NC(C)C)N═1)═C(N═2)C3═CC═C(F)C═C3)CC4)CC5═NC═CC═C5)═O)C |
| Example 570 | CC(OC(═O)N2CC═1C═CC═CC═1CC2C(═O)N6CCN(CC═4OC(C═3SC═C(C(═O)NC(C)C)N═3)═C(N═4)C5═CC═C(F)C═C5)CC6)(C)C |
| Example 571 | C1═CC(═CC═C1O)CC(C(═O)N5CCN(CC═3OC(C═2SC═C(C(═O)NC(C)C)N═2)═C(N═3)C4═CC═C(F)C═C4)CC5)NC(OC(C)(C)C)═O |
| Example 572 | N1═C(SC═C1C(═O)NC(C)C)C═3OC(CN2CCNCC2)═NC═3C4═CC═C(F)C═C4 |

### EXAMPLES 573-810

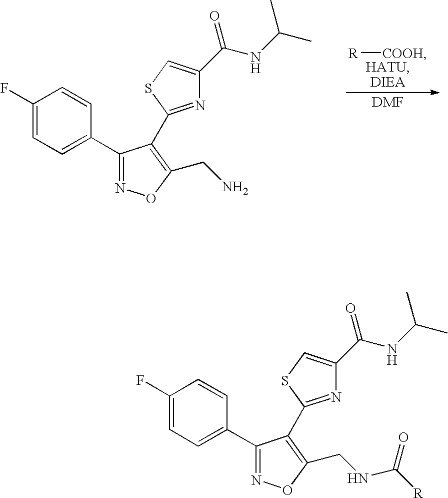

Utilizing the corresponding bromomethyl isoxazole prepared as described in Step 2 of Example 28, the starting amino methyl isoxazole shown in this example was prepared as described in Example 43, but omitting the Boc protection step. Where R—COOH is a carboxylic acid selected to afford Examples 573-810 were by General Procedure 1.

| | |
|---|---|
| Example 573 | CC(═O)NCC2═C(C═1SC═C(C(═O)NC(C)C)N═1)C(═NO2)C3═CC═C(F)C═C3 |
| Example 574 | O═C(C═O)NCC2═C(C═1SC═C(C(═O)NC(C)C)N═1)C(═NO2)C3═CC═C(F)C═C3 |
| Example 575 | O═C(CC#N)NCC2═C(C═1SC═C(C(═O)NC(C)C)N═1)C(═NO2)C3═CC═C(F)C═C3 |
| Example 576 | OC(C)C(═O)NCC2═C(C═1SC═C(C(═O)NC(C)C)N═1)C(═NO2)C3═CC═C(F)C═C3 |
| Example 577 | O═C(CC)C(═O)NCC2═C(C═1SC═C(C(═O)NC(C)C)N═1)C(═NO2)C3═CC═C(F)C═C3 |
| Example 578 | O═C(C(C)(C)C)NCC2═C(C═1SC═C(C(═O)NC(C)C)N═1)C(═NO2)C3═CC═C(F)C═C3 |
| Example 579 | O═C(CC(C)C)NCC2═C(C═1SC═C(C(═O)NC(C)C)N═1)C(═NO2)C3═CC═C(F)C═C3 |
| Example 580 | [H]N4C(C(═O)NCC2═C(C═1SC═C(C(═O)NC(C)C)N═1)C(═NO2)C3═CC═C(F)C═C3)═CN═C4 |
| Example 581 | [H]N1C(═NC═C1)C(═O)NCC3═C(C═2SC═C(C(═O)NC(C)C)N═2)C(═NO3)C4═CC═C(F)C═C4 |
| Example 582 | C1CC(═CC1)C(═O)NCC3═C(C═2SC═C(C(═O)NC(C)C)N═2)C(═NO3)C4═CC═C(F)C═C4 |
| Example 583 | FC(F)(C(═O)NCC2═C(C═1SC═C(C(═O)NC(C)C)N═1)C(═NO2)C3═CC═C(F)C═C3)F |
| Example 584 | C4CCOC4C(═O)NCC2═C(C═1SC═C(C(═O)NC(C)C)N═1)C(═NO2)C3═CC═C(F)C═C3 |
| Example 585 | O═C(C)NCC(═O)NCC2═C(C═1SC═C(C(═O)NC(C)C)N═1)C(═NO2)C3═CC═C(F)C═C3 |
| Example 586 | C═4C═CC═CC═4C(═O)NCC2═C(C═1SC═C(C(═O)NC(C)C)N═1)C(═NO2)C3═CC═C(F)C═C3 |
| Example 587 | C═4C═CN═CC═4C(═O)NCC2═C(C═1SC═C(C(═O)NC(C)C)N═1)C(═NO2)C3═CC═C(F)C═C3 |
| Example 588 | C1═CC(═CC═N1)C(═O)NCC3═C(C═2SC═C(C(═O)NC(C)C)N═2)C(═NO3)C4═CC═C(F)C═C4 |
| Example 589 | C1═NC(═CC═C1)C(═O)NCC3═C(C═2SC═C(C(═O)NC(C)C)N═2)C(═NO3)C4═CC═C(F)C═C4 |
| Example 590 | OC(CCl)C(═O)NCC2═C(C═1SC═C(C(═O)NC(C)C)N═1)C(═NO2)C3═CC═C(F)C═C3 |
| Example 591 | [H]N1N═C(C═C1C)C(═O)NCC3═C(C═2SC═C(C(═O)NC(C)C)N═2)C(═NO3)C4═CC═C(F)C═C4 |
| Example 592 | O═C(C═1N═CN(C═1)C)NCC3═C(C═2SC═C(C(═O)NC(C)C)N═2)C(═NO3)C4═CC═C(F)C═C4 |
| Example 593 | C1CC(CCC1)C(═O)NCC3═C(C═2SC═C(C(═O)NC(C)C)N═2)C(═NO3)C4═CC═C(F)C═C4 |
| Example 594 | N4═CSC═C4C(═O)NCC2═C(C═1SC═C(C(═O)NC(C)C)N═1)C(═NO2)C3═CC═C(F)C═C3 |
| Example 595 | O═C(OCC)CC(═O)NCC2═C(C═1SC═C(C(═O)NC(C)C)N═1)C(═NO2)C3═CC═C(F)C═C3 |
| Example 596 | CC4═CC(C(═O)NCC2═C(C═1SC═C(C(═O)NC(C)C)N═1)C(═NO2)C3═CC═C(F)C═C3)═CC═C4 |
| Example 597 | O═C(C1═CC═CC═C1C)NCC3═C(C═2SC═C(C(═O)NC(C)C)N═2)C(═NO3)C4═CC═C(F)C═C4 |
| Example 598 | O═C(C1═CC═C(C)C═C1)NCC3═C(C═2SC═C(C(═O)NC(C)C)N═2)C(═NO3)C4═CC═C(F)C═C4 |
| Example 599 | OC═1C═CC═C(C═1)C(═O)NCC3═C(C═2SC═C(C(═O)NC(C)C)N═2)C(═NO3)C4═CC═C(F)C═C4 |
| Example 600 | OC═4C(C(═O)NCC2═C(C═1SC═C(C(═O)NC(C)C)N═1)C(═NO2)C3═CC═C(F)C═C3)═CC═CC═4 |
| Example 601 | C═1C═C(C═CC═1F)C(═O)NCC3═C(C═2SC═C(C(═O)NC(C)C)N═2)C(═NO3)C4═CC═C(F)C═C4 |
| Example 602 | C1═CC═C(C═C1F)C(═O)NCC3═C(C═2SC═C(C(═O)NC(C)C)N═2)C(═NO3)C4═CC═C(F)C═C4 |
| Example 603 | CCCCCCCC(═O)NCC2═C(C═1SC═C(C(═O)NC(C)C)N═1)C(═NO2)C3═CC═C(F)C═C3 |
| Example 604 | O═C(C1═CC═C(C#N)C═C1)NCC3═C(C═2SC═C(C(═O)NC(C)C)N═2)C(═NO3)C4═CC═C(F)C═C4 |
| Example 605 | O═C(C1═CC═CC(C#N)═C1)NCC3═C(C═2SC═C(C(═O)NC(C)C)N═2)C(═NO3)C4═CC═C(F)C═C4 |
| Example 606 | C═4C═CC═CC═4/C═C/C(═O)NCC2═C(C═1SC═C(C(═O)NC(C)C)N═1)C(═NO2)C3═CC═C(F)C═C3 |
| Example 607 | O═C(C1═CC═CC═C1C═O)NCC3═C(C═2SC═C(C(═O)NC(C)C)N═2)C(═NO3)C4═CC═C(F)C═C4 |
| Example 608 | O═C(C1═CC═C(C═O)C═C1)NCC3═C(C═2SC═C(C(═O)NC(C)C)N═2)C(═NO3)C4═CC═C(F)C═C4 |
| Example 609 | CC1═CC(C)═CC═C1C(═O)NCC3═C(C═2SC═C(C(═O)NC(C)C)N═2)C(═NO3)C4═CC═C(F)C═C4 |
| Example 610 | C═1C═CC═C(C═1C)CC(═O)NCC3═C(C═2SC═C(C(═O)NC(C)C)N═2)C(═NO3)C4═CC═C(F)C═C4 |
| Example 611 | C═4C═CC(═C(C═4C(═O)NCC2═C(C═1SC═C(C(═O)NC(C)C)N═1)C(═NO2)C3═CC═C(F)C═C3)C)C |
| Example 612 | CC1═C(C═C(C)C═C1)C(═O)NCC3═C(C═2SC═C(C(═O)NC(C)C)N═2)C(═NO3)C4═CC═C(F)C═C4 |
| Example 613 | CC1═CC(═CC(C)═C1)C(═O)NCC3═C(C═2SC═C(C(═O)NC(C)C)N═2)C(═NO3)C4═CC═C(F)C═C4 |
| Example 614 | CC═4C(═CC═C(C(═O)NCC2═C(C═1SC═C(C(═O)NC(C)C)N═1)C(═NO2)C3═CC═C(F)C═C3)C═4)C |
| Example 615 | C═1C═C(C═CC═1CC)C(═O)NCC3═C(C═2SC═C(C(═O)NC(C)C)N═2)C(═NO3)C4═CC═C(F)C═C4 |
| Example 616 | COC4═CC═C(C(═O)NCC2═C(C═1SC═C(C(═O)NC(C)C)N═1)C(═NO2)C3═CC═C(F)C═C3)C═C4 |
| Example 617 | CC1═CC═CC(═C1O)C(═O)NCC3═C(C═2SC═C(C(═O)NC(C)C)N═2)C(═NO3)C4═CC═C(F)C═C4 |
| Example 618 | OC═1C═CC═CC═1CC(═O)NCC3═C(C═2SC═C(C(═O)NC(C)C)N═2)C(═NO3)C4═CC═C(F)C═C4 |
| Example 619 | C1═CC═C(C═C1O)CC(═O)NCC3═C(C═2SC═C(C(═O)NC(C)C)N═2)C(═NO3)C4═CC═C(F)C═C4 |
| Example 620 | C1═CC(═C(C═C1C)C(═O)NCC3═C(C═2SC═C(C(═O)NC(C)C)N═2)C(═NO3)C4═CC═C(F)C═C4)O |
| Example 621 | OC1═CC═C(O)C═C1C(═O)NCC3═C(C═2SC═C(C(═O)NC(C)C)N═2)C(═NO3)C4═CC═C(F)C═C4 |
| Example 622 | OC1═C(O)C═CC═C1C(═O)NCC3═C(C═2SC═C(C(═O)NC(C)C)N═2)C(═NO3)C4═CC═C(F)C═C4 |
| Example 623 | OC═4C(C(═O)NCC2═C(C═1SC═C(C(═O)NC(C)C)N═1)C(═NO2)C3═CC═C(F)C═C3)═CC═C(C═4)O |
| Example 624 | FC═1C(C)═CC═C(C═1)C(═O)NCC3═C(C═2SC═C(C(═O)NC(C)C)N═2)C(═NO3)C4═CC═C(F)C═C4 |
| Example 625 | CC═4C(C(═O)NCC2═C(C═1SC═C(C(═O)NC(C)C)N═1)C(═NO2)C3═CC═C(F)C═C3)═CC(═CC═4)F |
| Example 626 | ClC1═CC═CC═C1C(═O)NCC3═C(C═2SC═C(C(═O)NC(C)C)N═2)C(═NO3)C4═CC═C(F)C═C4 |
| Example 627 | O═C(C1═CN═C(Cl)C═C1)NCC3═C(C═2SC═C(C(═O)NC(C)C)N═2)C(═NO3)C4═CC═C(F)C═C4 |
| Example 628 | ClC═1N═CC═CC═1C(═O)NCC3═C(C═2SC═C(C(═O)NC(C)C)N═2)C(═NO3)C4═CC═C(F)C═C4 |
| Example 629 | ClC1═NC═CC(═C1)C(═O)NCC3═C(C═2SC═C(C(═O)NC(C)C)N═2)C(═NO3)C4═CC═C(F)C═C4 |
| Example 630 | O═C(C1═NC═CC(═C1)Cl)NCC3═C(C═2SC═C(C(═O)NC(C)C)N═2)C(═NO3)C4═CC═C(F)C═C4 |
| Example 631 | O═C(C1═C(C═NC═C1)Cl)NCC3═C(C═2SC═C(C(═O)NC(C)C)N═2)C(═NO3)C4═CC═C(F)C═C4 |
| Example 632 | FC═1C═CC═C(F)C═1C(═O)NCC3═C(C═2SC═C(C(═O)NC(C)C)N═2)C(═NO3)C4═CC═C(F)C═C4 |
| Example 633 | O═C(C1═CC═CC(F)═C1F)NCC3═C(C═2SC═C(C(═O)NC(C)C)N═2)C(═NO3)C4═CC═C(F)C═C4 |
| Example 634 | O═C(C1═CC═C(F)C═C1F)NCC3═C(C═2SC═C(C(═O)NC(C)C)N═2)C(═NO3)C4═CC═C(F)C═C4 |
| Example 635 | FC═1C═C(F)C═C(C═1)C(═O)NCC3═C(C═2SC═C(C(═O)NC(C)C)N═2)C(═NO3)C4═CC═C(F)C═C4 |
| Example 636 | O═C(C1═CC═C(F)C(═C1)F)NCC3═C(C═2SC═C(C(═O)NC(C)C)N═2)C(═NO3)C4═CC═C(F)C═C4 |
| Example 637 | O═C(CC(CC(C)(C)C)C)NCC2═C(C═1SC═C(C(═O)NC(C)C)N═1)C(═NO2)C3═CC═C(F)C═C3 |
| Example 638 | C═4C5═CC═CC═C5OC═4C(═O)NCC2═C(C═1SC═C(C(═O)NC(C)C)N═1)C(═NO2)C3═CC═C(F)C═C3 |
| Example 639 | O═C(C2CCC1═CC═CC═C12)NCC4═C(C═3SC═C(C(═O)NC(C)C)N═3)C(═NO4)C5═CC═C(F)C═C5 |
| Example 640 | ClC(C(═O)NCC2═C(C═1SC═C(C(═O)NC(C)C)N═1)C(═NO2)C3═CC═C(F)C═C3)(Cl)Cl |
| Example 641 | O═C(/C═C/C═1C(O)═CC═CC═1)NCC3═C(C═2SC═C(C(═O)NC(C)C)N═2)C(═NO3)C4═CC═C(F)C═C4 |
| Example 642 | O═C(C1═CC═C(C(C)═O)C═C1)NCC3═C(C═2SC═C(C(═O)NC(C)C)N═2)C(═NO3)C4═CC═C(F)C═C4 |
| Example 643 | O═C(/C═C/C1═CC(O)═CC═C1)NCC3═C(C═2SC═C(C(═O)NC(C)C)N═2)C(═NO3)C4═CC═C(F)C═C4 |
| Example 644 | CC(═O)C1═CC═CC(═C1)C(═O)NCC3═C(C═2SC═C(C(═O)NC(C)C)N═2)C(═NO3)C4═CC═C(F)C═C4 |
| Example 645 | CC(═O)C1═CC═CC═C1C(═O)NCC3═C(C═2SC═C(C(═O)NC(C)C)N═2)C(═NO3)C4═CC═C(F)C═C4 |
| Example 646 | C4═CC(═CC═C4C(═O)NCC2═C(C═1SC═C(C(═O)NC(C)C)N═1)C(═NO2)C3═CC═C(F)C═C3)C(C)C |
| Example 647 | CC═1C═CC═CC═1CCC(═O)NCC3═C(C═2SC═C(C(═O)NC(C)C)N═2)C(═NO3)C4═CC═C(F)C═C4 |
| Example 648 | O═C(CCC═1C═C(C)C═CC═1)NCC3═C(C═2SC═C(C(═O)NC(C)C)N═2)C(═NO3)C4═CC═C(F)C═C4 |
| Example 649 | O═C(C(CC)C1═CC═CC═C1)NCC3═C(C═2SC═C(C(═O)NC(C)C)N═2)C(═NO3)C4═CC═C(F)C═C4 |
| Example 650 | CC═1C═C(C)C═C(C═1C(═O)NCC3═C(C═2SC═C(C(═O)NC(C)C)N═2)C(═NO3)C4═CC═C(F)C═C4)C |
| Example 651 | C1═CC(═CC═C1CCC)C(═O)NCC3═C(C═2SC═C(C(═O)NC(C)C)N═2)C(═NO3)C4═CC═C(F)C═C4 |
| Example 652 | O═C(C═1C═CC═2OCOC═2C═1)NCC4═C(C═3SC═C(C(═O)NC(C)C)N═3)C(═NO4)C5═CC═C(F)C═C5 |
| Example 653 | O═C(/C═C/C═1C(F)═CC═CC═1)NCC3═C(C═2SC═C(C(═O)NC(C)C)N═2)C(═NO3)C4═CC═C(F)C═C4 |
| Example 654 | C═1C═CC═C(C═1OCC)C(═O)NCC3═C(C═2SC═C(C(═O)NC(C)C)N═2)C(═NO3)C4═CC═C(F)C═C4 |
| Example 655 | CC═1C═CC═CC═1OCC(═O)NCC3═C(C═2SC═C(C(═O)NC(C)C)N═2)C(═NO3)C4═CC═C(F)C═C4 |
| Example 656 | O═C(CCC1═CC═CC═C1O)NCC3═C(C═2SC═C(C(═O)NC(C)C)N═2)C(═NO3)C4═CC═C(F)C═C4 |
| Example 657 | O═C(CC1═C(OC)C═CC═C1)NCC3═C(C═2SC═C(C(═O)NC(C)C)N═2)C(═NO3)C4═CC═C(F)C═C4 |
| Example 658 | O═C(C(C═1C═CC(O)═CC═1)C)NCC3═C(C═2SC═C(C(═O)NC(C)C)N═2)C(═NO3)C4═CC═C(F)C═C4 |
| Example 659 | O═[N+]([O−])C1═CC═CC═C1C(═O)NCC3═C(C═2SC═C(C(═O)NC(C)C)N═2)C(═NO3)C4═CC═C(F)C═C4 |
| Example 660 | O═[N+]([O−])C═1C═CC═C(C═1)C(═O)NCC3═C(C═2SC═C(C(═O)NC(C)C)N═2)C(═NO3)C4═CC═C(F)C═C4 |
| Example 661 | COC1═C(C(O)═CC═C1)C(═O)NCC3═C(C═2SC═C(C(═O)NC(C)C)N═2)C(═NO3)C4═CC═C(F)C═C4 |
| Example 662 | OC1═C(C═C(OC)C═C1)C(═O)NCC3═C(C═2SC═C(C(═O)NC(C)C)N═2)C(═NO3)C4═CC═C(F)C═C4 |
| Example 663 | OC═1C═C(OC)C═CC═1 C(═O)NCC3═C(C═2SC═C(C(═O)NC(C)C)N═2)C(═NO3)C4═CC═C(F)C═C4 |
| Example 664 | O═C(C1═CC(O)═C(OC)C═C1)NCC3═C(C═2SC═C(C(═O)NC(C)C)N═2)C(═NO3)C4═CC═C(F)C═C4 |
| Example 665 | OC═1C(O)═C(C═C(C═1)C(═O)NCC3═C(C═2SC═C(C(═O)NC(C)C)N═2)C(═NO3)C4═CC═C(F)C═C4)O |
| Example 666 | FC4═CC(CC(═O)NCC2═C(C═1SC═C(C(═O)NC(C)C)N═1)C(═NO2)C3═CC═C(F)C═C3)═CC═C4O |
| Example 667 | O═C(C═1C═C(N═C(C═1)Cl)C)NCC3═C(C═2SC═C(C(═O)NC(C)C)N═2)C(═NO3)C4═CC═C(F)C═C4 |
| Example 668 | FC═1C═CC═C(C═1CC(═O)NCC3═C(C═2SC═C(C(═O)NC(C)C)N═2)C(═NO3)C4═CC═C(F)C═C4)F |
| Example 669 | O═C(C2═CC═CC1═CC═CC═C12)NCC4═C(C═3SC═C(C(═O)NC(C)C)N═3)C(═NO4)C5═CC═C(F)C═C5 |
| Example 670 | O═C(C1═CC═C(Cl)C═C1O)NCC3═C(C═2SC═C(C(═O)NC(C)C)N═2)C(═NO3)C4═CC═C(F)C═C4 |
| Example 671 | OC═4C(C(═O)NCC2═C(C═1SC═C(C(═O)NC(C)C)N═1)C(═NO2)C3═CC═C(F)C═C3)═CC(═CC═4)Cl |
| Example 672 | O═C(C1═CC═2C═CC═NC═2C═C1)NCC4═C(C═3SC═C(C(═O)NC(C)C)N═3)C(═NO4)C5═CC═C(F)C═C5 |
| Example 673 | OC4═NC═C(C(═O)NCC2═C(C═1SC═C(C(═O)NC(C)C)N═1)C(═NO2)C3═CC═C(F)C═C3)C═C4Cl |
| Example 674 | O═C(C1═CC═C(F)C═C1Cl)NCC3═C(C═2SC═C(C(═O)NC(C)C)N═2)C(═NO3)C4═CC═C(F)C═C4 |
| Example 675 | FC═1C═CC═C(C═1C(═O)NCC3═C(C═2SC═C(C(═O)NC(C)C)N═2)C(═NO3)C4═CC═C(F)C═C4)Cl |
| Example 676 | CC(C)(OC(NCC(═O)NCC2═C(C═1SC═C(C(═O)NC(C)C)N═1)C(═NO2)C3═CC═C(F)C═C3)═O)C |
| Example 677 | O═C(C═1OC2═CC═CC═C2C═1C)NCC4═C(C═3SC═C(C(═O)NC(C)C)N═3)C(═NO4)C5═CC═C(F)C═C5 |
| Example 678 | C═1C2═C(C═CC═1)CC(C2)CC(═O)NCC4═C(C═3SC═C(C(═O)NC(C)C)N═3)C(═NO4)C5═CC═C(F)C═C5 |
| Example 679 | C4CC5═C(CC4C(═O)NCC2═C(C═1SC═C(C(═O)NC(C)C)N═1)C(═NO2)C3═CC═C(F)C═C3)C═CC═C5 |
| Example 680 | O═C(/C═C/C═1C═CC═CC═1OC)NCC3═C(C═2SC═C(C(═O)NC(C)C)N═2)C(═NO3)C4═CC═C(F)C═C4 |
| Example 681 | O═C(C1═CSC2═C1C═CC═C2)NCC4═C(C═3SC═C(C(═O)NC(C)C)N═3)C(═NO4)C5═CC═C(F)C═C5 |
| Example 682 | C═1C═C2C═C(SC2═CC═1)C(═O)NCC4═C(C═3SC═C(C(═O)NC(C)C)N═3)C(═NO4)C5═CC═C(F)C═C5 |
| Example 683 | O═C(C1═CC═C(CCCC)C═C1)NCC3═C(C═2SC═C(C(═O)NC(C)N═2)C(═NO3)C4═CC═C(F)C═C4 |
| Example 684 | O═C(C1═CC═C(C(C)(C)C)C═C1)NCC3═C(C═2SC═C(C(═O)NC(C)C)N═2)C(═NO3)C4═CC═C(F)C═C4 |
| Example 685 | O═C(C1═CC═C(N(C═O)C)C═C1)NCC3═C(C═2SC═C(C(═O)NC(C)C)N═2)C(═NO3)C4═CC═C(F)C═C4 |
| Example 686 | O═C(C═1C═C(NC(C)═O)C═CC═1)NCC3═C(C═2SC═C(C(═O)NC(C)C)N═2)C(═NO3)C4═CC═C(F)C═C4 |
| Example 687 | O═C(COC═1C(C═O)═CC═CC═1)NCC3═C(C═2SC═C(C(═O)NC(C)C)N═2)C(═NO3)C4═CC═C(F)C═C4 |
| Example 688 | CC(═O)OC1═CC═C(C═C1)C(═O)NCC3═C(C═2SC═C(C(═O)NC(C)C)N═2)C(═NO3)C4═CC═C(F)C═C4 |
| Example 689 | O═C(OC)C4═CC═C(C(═O)NCC2═C(C═1SC═C(C(═O)NC(C)C)N═1)C(═NO2)C3═CC═C(F)C═C3)C═C4 |
| Example 690 | C4═C(C═CC═C4C(═O)NCC2═C(C═1SC═C(C(═O)NC(C)C)N═1)C(═NO2)C3═CC═C(F)C═C3)OC(═O)C |
| Example 691 | CC(═O)OC1═CC═CC═C1C(═O)NCC3═C(C═2SC═C(C(═O)NC(C)C)N═2)C(═NO3)C4═CC═C(F)C═C4 |
| Example 692 | O1C═2C═CC(═CC═2OC1)CC(═O)NCC4═C(C═3SC═C(C(═O)NC(C)C)N═3)C(═NO4)C5═CC═C(F)C═C5 |
| Example 693 | C1═CC(═CC═C1OCCC)C(═O)NCC3═C(C═2SC═C(C(═O)NC(C)C)N═2)C(═NO3)C4═CC═C(F)C═C4 |
| Example 694 | O═C(C1═CC═C(OC(C)C)C═C1)NCC3═C(C═2SC═C(C(═O)NC(C)C)N═2)C(═NO3)C4═CC═C(F)C═C4 |
| Example 695 | [O−][N+](═O)C1═C(C(═CC═C1)C)C(O)NCC3═C(C═2SC═C(C(═O)NC(C)C)N═2)C(═NO3)C4═CC═C(F)C═C4 |
| Example 696 | O═[N+]([O−])C1═C(C═CC═C1C(═O)NCC3═C(C═2SC═C(C(═O)NO(C)C)N═2)C(═NO3)C4═CC═C(F)C═C4)C |
| Example 697 | [O−][N+](═O)C1═CC═C(C═C1C(═O)NCC3═C(C═2SC═C(C(═O)NC(C)C)N═2)C(═NO3)C4═CC═C(F)C═C4)C |
| Example 698 | CC═4C(C(═O)NCC2═C(C═1SC═C(C(═O)NC(C)C)N═1)C(═NO2)C3═CC═C(F)C═C3)═CC═CC═4 [N+]([O−])═O |
| Example 699 | C1═CC(═C(C═C1OC)C(═O)NCC3═C(C═2SC═C(C(═O)NC(C)C)N═2)C(═NO3)C4═CC═C(F)C═C4)OC |
| Example 700 | COC1═CC(OC)═CC═C1C(═O)NCC3═C(C═2SC═C(C(═O)NC(C)C)N═2)C(═NO3)C4═CC═C(F)C═C4 |
| Example 701 | COC═1C(═C(OC)C═CC═1)C(═O)NCC3═C(C═2SC═C(C(═O)NC(C)C)N═2)C(═NO3)C4═CC═C(F)C═C4 |
| Example 702 | C═1C(═CC(═CC═1OC)C(═O)NCC3═C(C═2SC═C(C(═O)NC(C)C)N═2)C(═NO3)C4═CC═C(F)C═C4)OC |
| Example 703 | C═1C═CC(═C(C═1OC)OC)C(═O)NCC3═C(C═2SC═C(C(═O)NC(C)C)N═2)C(═NO3)C4═CC═C(F)C═C4 |
| Example 704 | CC4═CC═C(SCC(═O)NCC2═C(C═1SC═C(C(═O)NC(C)C)N═1)C(═NO2)C3═CC═C(F)C═C3)C═C4 |
| Example 705 | O═C(/C═C/C═1C═CC(Cl)═CC═1)NCC3═C(C═2SC═C(C(═O)NC(C)C)N═2)C(═NO3)C4═CC═C(F)C═C4 |
| Example 706 | O═C(/C═C/C═1C(Cl)═CC═CC═1)NCC3═C(C═2SC═C(C(═O)NC(C)C)N═2)C(═NO3)C4═CC═C(F)C═C4 |
| Example 707 | [O−][N+](C1═CC═C(C═C1O)C(═O)NCC3═C(C═2SC═C(C(═O)NC(C)C)N═2)C(═NO3)C4═CC═C(F)C═C4)═O |
| Example 708 | O═C(C1═CC(O)═CC═C1 [N+]([O−])═O)NCC3═C(C═2SC═C(C(═O)NC(C)C)N═2)C(═NO3)C4═CC═C(F)C═C4 |
| Example 709 | OC4═CC═C(C═C4C(═O)NCC2═C(C═1SC═C(C(═O)NC(C)C)N═1)C(═NO2)C3═CC═C(F)C═C3)[N+](═O)[O−] |
| Example 710 | O═[N+]([O−])C1═CC═C(C(═C1)C(═O)NCC3═C(C═2SC═C(C(═O)NC(C)C)N═2)C(═NO3)C4═CC═C(F)C═C4)F |
| Example 711 | O═[N+]([O−])C1═CC═C(C═C1C(═O)NCC3═C(C═2SC═C(C(═O)NC(C)C)N═2)C(═NO3)C4═CC═C(F)C═C4)F |
| Example 712 | O═[N+]([O−])C═1C(═CC═C(C═1)C(═O)NCC3═C(C═2SC═C(C(═O)NC(C)C)N═2)C(═NO3)C4═CC═C(F)C═C4)F |
| Example 713 | COC1═CC═C(Cl)C═C1C(═O)NCC3═C(C═2SC═C(C(═O)NC(C)C)N═2)C(═NO3)C4═CC═C(F)C═C4 |
| Example 714 | ClC═1C═C(OC)C(═CC═1)C(═O)NCC3═C(C═2SC═C(C(═O)NC(C)C)N═2)C(═NO3)C4═CC═C(F)C═C4 |
| Example 715 | O═C(CC1═CC(═C(O)C═C1)Cl)NCC3═C(C═2SC═C(C(═O)NC(C)C)N═2)C(═NO3)C4═CC═C(F)C═C4 |
| Example 716 | O═C(C═1C═C(N═C(C═1)Cl)OC)NCC3═C(C═2SC═C(C(═O)NC(C)C)N═2)C(═NO3)C4═CC═C(F)C═C4 |
| Example 717 | OC5═CC═C1C(C═CC(═C1)C(C═O)NCC3═C(C═2SC═C(C(═O)NC(C)C)N═2)C(═NO3)C4═CC═C(F)C═C4)═C5 |
| Example 718 | O═C(C═1C2═CC═CC═C2C═CC═1O)NCC4═C(C═3SC═C(C(═O)NC(C)C)N═3)C(═NO4)C5═CC═C(F)C═C5 |
| Example 719 | O═C(C1═CC2═CC═CC═C2C═C1O)NCC4═C(C═3SC═C(C(═O)NC(C)C)N═3)C(═NO4)C5═CC═C(F)C═C5 |
| Example 720 | C1═CC═2C(C═C1)═CC═C(C═2O)C(═O)NCC4═C(C═3SC═C(C(═O)NC(C)C)N═3)C(═NO4)C5═CC═C(F)C═C5 |
| Example 721 | OC1═CC═CC2═CC═C(N═C12)C(═O)NCC4═C(C═3SC═C(C(═O)NC(C)C)N═3)C(═NO4)C5═CC═C(F)C═C5 |
| Example 722 | CC(C)(OC(NC(C(═O)NCC2═C(C═1SC═C(C(═O)NC(C)C)N═1)C(═NO2)C3═CC═C(F)C═C3)C)═O)C |
| Example 723 | CC(C)(OC(NC(C(═O)NCC2═C(C═1SC═C(C(═O)NC(C)C)N═1)C(═NO2)C3═CC═C(F)C═C3)C)═O)C |
| Example 724 | O═C(C═1C(═CC═CC═1)C(F)(F)F)NCC3═C(C═2SC═C(C(═O)NC(C)C)N═2)C(═NO3)C4═CC═C(F)C═C4 |
| Example 725 | [H]N2C1═CC═CC═C1N═C2CCC(═O)NCC4═C(C═3SC═C(C(═O)NC(C)C)N═3)C(═NO4)C5═CC═C(F)C═C5 |
| Example 726 | ClC1═C(Cl)C═CC═C1C(═O)NCC3═C(C═2SC═C(C(═O)NC(C)C)N═2)C(═NO3)C4═CC═C(F)C═C4 |
| Example 727 | ClC1═CC(Cl)═CC═C1C(═O)NCC3═C(C═2SC═C(C(═O)NC(C)C)N═2)C(═NO3)C4═CC═C(F)C═C4 |
| Example 728 | ClC═4C(C(═O)NCC2═C(C═1SC═C(C(═O)NC(C)C)N═1)C(═NO2)C3═CC═C(F)C═C3)═CC(═CC═4)Cl |
| Example 729 | O═C(C1═C(C(═C(C(═C1)F)F)O)F)NCC3═C(C═2SC═C(C(═O)NC(C)C)N═2)C(═NO3)C4═CC═C(F)C═C4 |
| Example 730 | O1C═2C═CC(═CC═2OC1)/ C═C/C(═O)NCC4═C(C═3SC═C(C(C(═O)NC(C)C)N═3)C(═NO4)C5═CC═C(F)C═C5 |
| Example 731 | C1═CC(═CC═C1C)C(CCC(═O)NCC3═C(C═2SC═C(C(═O)NC(C)C)N═2)C(═NO3)C4═CC═C(F)C═C4)═O |
| Example 732 | O═C(C1═CC═C(CCCCC)C═C1)NCC3═C(C═2SC═C(C(═O)NC(C)C)N═2)C(═NO3)C4═CC═C(F)C═C4 |
| Example 733 | O═C(C1═CC(═C(F)C═C1Cl)F)NCC3═C(C═2SC═C(C(═O)NC(C)C)N═2)C(═NO3)C4═CC═C(F)C═C4 |
| Example 734 | FC1═CC(Cl)═C(C═C1C(═O)NCC3═C(C═2SC═C(C(═O)NC(C)C)N═2)C(═NO3)C4═CC═C(F)C═C4)F |
| Example 735 | O═C(C1═CC═C(N(CC)CC)C═C1)NCC3═C(C═2SC═C(C(═O)NC(C)C)N═2)C(═NO3)C4═CC═C(F)C═C4 |
| Example 736 | COC4═CC(/C═C/C(═O)NCC2═C(C═1SC═C(C(═O)NC(C)C)N═1)C(═NO2)C3═CC═C(F)C═C3)═CC═C4O |
| Example 737 | O═C(/C═C/C1═CC(O)═C(OC)C═C1)NCC3═C(C═2SC═C(C(═O)NC(C)C)N═2)C(═NO3)C4═CC═C(F)C═C4 |
| Example 738 | O═C(C1═CC═C(OCCCC)C═C1)NCC3═C(C═2SC═C(C(C═O)NC(C)C)N═2)C(═NO3)C4═CC═C(F)C═C4 |
| Example 739 | O═C(C═2OC1═C(C═C(Cl)C═C1)C═2)NCC4═C(C═3SC═C(C(═O)NC(C)C)N═3)C(═NO4)C5═CC═C(F)C═C5 |
| Example 740 | C1═CC(═C(C═C1OC)C(═O)NCC3═C(C═2SC═C(C(═O)NC(C)C)N═2)C(═NO3)C4═CC═C(F)C═C4)[N+]([O−])═O |
| Example 741 | O═C(C1═CC(OC)═C(O)C(OC)═C1)NCC3═C(C═2SC═C(C(═O)NC(C)C)N═2)C(═NO3)C4═CC═C(F)C═C4 |
| Example 742 | C5═CC═CC═C5C═4C(C(═O)NCC2═C(C═1SC═C(C(═O)NC(C)C)N═1)C(═NO2)C3═CC═C(F)C═C3)═CC═CC═4 |
| Example 743 | BrC1═CC═CC(═C1)C(═O)NCC3═C(C═2SC═C(C(═O)NC(C)C)N═2)C(═NO3)C4═CC═C(F)C═C4 |
| Example 744 | C═1C═C(C═CC═1Br)C(═O)NCC3═C(C═2SC═C(C(═O)NC(C)C)N═2)C(═NO3)C4═CC═C(F)C═C4 |
| Example 745 | [O−][N+](═O)C1═C(C═CC═C1C(═O)NCC3═C(C═2SC═C(C(═O)NC(C)C)N═2)C(═NO3)C4═CC═C(F)C═C4)Cl |
| Example 746 | O═[N+]([O−])C1═CC═C(C(═C1)C(═O)NCC3═C(C═2SC═C(C(═O)NC(C)C)N═2)C(═NO3)C4═CC═C(F)C═C4)Cl |
| Example 747 | O═C(C1═C(C═C([N+]([O−])═O)C═C1)Cl)NCC3═C(C═2SC═C(C(═O)NC(C)C)N═2)C(═NO3)C4═CC═C(F)C═C4 |
| Example 748 | O═[N+]([O−])C1═CC(═CC═C1C(═O)NCC3═C(C═2SC═C(C(═O)NC(C)C)N═2)C(═NO3)C4═CC═C(F)C═C4)Cl |
| Example 749 | O═[N+]([O−])C1═CC(═CC═C1Cl)C(═O)NCC3═C(C═2SC═C(C(═O)NC(C)C)N═2)C(═NO3)C4═CC═C(F)C═C4 |
| Example 750 | O═C(C1═CC(Cl)═CC═C1 [N+](═O)[O−])NCC3═C(C═2SC═C(C(═O)NC(C)C)N═2)C(═NO3)C4═CC═C(F)C═C4 |
| Example 751 | C═4C═CC(═NC═4C(═O)NCC2═C(C═1SC═C(C(═O)NC(C)C)N═1)C(═NO2)C3═CC═C(F)C═C3)Br |
| Example 752 | O═C(C═1C═NC═C(Br)C═1)NCC3═C(C═2SC═C(C(═O)NC(C)C)N═2)C(═NO3)C4═CC═C(F)C═C4 |
| Example 753 | [O−][N+](═O)C═4C(C(═O)NCC2═C(C═1SC═C(C(═O)NC(C)C)N═1)C(═NO2)C3═CC═C(F)C═C3)═CC(F)═C(F)C═4 |
| Example 754 | FC(F)(C═1C═CC(═CC═1)CC(═O)NCC3═C(C═2SC═C(C(═O)NC(C)C)N═2)C(═NO3)C4═CC═C(F)C═C4)F |
| Example 755 | CC(C)CC4═CC═C(C(C(═O)NCC2═C(C═1SC═C(C(═O)NC(C)C)N═1)C(═NO2)C3═CC═C(F)C═C3)C)C═C4 |
| Example 756 | ClC1═C(O)C(═CC(═C1)C(═O)NCC3═C(C═2SC═C(C(═O)NC(C)C)N═2)C(═NO3)C4═CC═C(F)C═C4)Cl |
| Example 757 | FC═1C(F)═C(C(═C(C═1F)C(═O)NCC3═C(C═2SC═C(C(═O)NC(C)C)N═2)C(═NO3)C4═CC═C(F)C═C4)F)F |
| Example 758 | O═C(C1═C(OC)C═C(C═C1OC)OC)NCC3═C(C═2SC═C(C(═O)NC(C)C)N═2)C(═NO3)C4═CC═C(F)C═C4 |
| Example 759 | COC1═CC(═C(C═C1OC)OC)C(═O)NCC3═C(C═2SC═C(C(═O)NC(C)C)N═2)C(═NO3)C4═CC═C(F)C═C4 |
| Example 760 | COC1═C(OC)C(═CC(═C1)C(═O)NCC3═C(C═2SC═C(C(═O)NC(C)C)N═2)C(═NO3)C4═CC═C(F)C═C4)OC |
| Example 761 | COC1═C(OC)C═CC(═C1OC)C(═O)NCC3═C(C═2SC═C(C(═O)NC(C)C)N═2)C(═NO3)C4═CC═C(F)C═C4 |
| Example 762 | C═1C═C(C═CC═1)CC5═C(C(═O)NCC3═C(C═2SC═C(C(═O)NC(C)C)N═2)C(═NO3)C4═CC═C(F)C═C4)C═CC═C5 |
| Example 763 | O═C(C2═CC═CC(OC1═CC═CC═C1)═C2)NCC4═C(C═3SC═C(C(═O)NC(C)C)N═3)C(═NO4)C5═CC═C(F)C═C5 |
| Example 764 | C1═CC(═CC═C1)OC5═C(C(═O)NCC3═C(C═2SC═C(C(═O)NC(C)C)N═2)C(═NO3)C4═CC═C(F)C═C4)C═CC═C5 |
| Example 765 | O═C(C2═CC═C(OC1═CC═CC═C1)C═C2)NCC4═C(C═3SC═C(C(═O)NC(C)C)N═3)C(═NO4)C5═CC═C(F)C═C5 |
| Example 766 | C1═CC(═CC═C1O)C2═CC═C(C═C2)C(═O)NCC4═C(C═3SC═C(C(═O)NC(C)C)N═3)C(═NO4)C5═CC═C(F)C═C5 |
| Example 767 | BrC═1C═CC(═CC═1C)C(═O)NCC3═C(C═2SC═C(C(═O)NC(C)C)N═2)C(═NO3)C4═CC═C(F)C═C4 |
| Example 768 | BrC1═C(C)C═CC(═C1)C(═O)NCC3═C(C═2SC═C(C(═O)NC(C)C)N═2)C(═NO3)C4═CC═C(F)C═C4 |
| Example 769 | O═C(CC1═CC═C(Br)C═C1)NCC3═C(C═2SC═C(C(═O)NC(C)C)N═2)C(═NO3)C4═CC═C(F)C═C4 |
| Example 770 | CC(OC(═O)N1C(CCC1)C(═O)NCC3═C(C═2SC═C(C(═O)NC(C)C)N═2)C(═NO3)C4═CC═C(F)C═C4)(C)C |
| Example 771 | O═C(/C═C/C1═C(C(F)(F)F)C═CC═C1)NCC3═C(C═2SC═C(C(═O)NC(C)C)N═2)C(═NO3)C4═CC═C(F)C═C4 |
| Example 772 | O═C(C1═C(OCC)C═CC2═C1C═CC═C2)NCC4═C(C═3SC═C(C(═O)NC(C)C)N═3)C(═NO4)C5═CC═C(F)C═C5 |
| Example 773 | CC(C)(OC(NC(C(═O)NCC2═C(C═1SC═C(C(═O)NC(C)C)N═1)C(═NO2)C3═CC═C(F)C═C3)C(C)C)═O)C |
| Example 774 | CC(C)(OC(NC(C(═O)NCC2═C(C═1SC═C(C(═O)NC(C)C)N═1)C(═NO2)C3═CC═C(F)C═C3)C(C)C)═O)C |
| Example 775 | O═C(CCC═1C═C(C═CC═1)C(F)(F)F)NCC3═C(C═2SC═C(C(═O)NC(C)C)N═2)C(═NO3)C4═CC═C(F)C═C4 |
| Example 776 | O═C(C═2C═CC(N1N═C(C)CC1═O)═CC═2)NCC4═C(C═3SC═C(C(═O)NC(C)C)N═3)C(═NO4)C5═CC═C(F)C═C5 |
| Example 777 | CC4═C(C(═O)NCC2═C(C═1SC═C(C(═O)NC(C)C)N═1)C(═NO2)C3═CC═C(F)C═C3)SC(═N4)Br |
| Example 778 | C1═CC2═C(C═C1)C═C6C(═C2C(═O)NCC4═C(C═3SC═C(C(═O)NC(C)C)N═3)C(═NO4)C5═CC═C(F)C═C5)C═CC═C6 |
| Example 779 | CC(C)C4═CC(C(═O)NCC2═C(C═1SC═C(C(═O)NC(C)C)N═1)C(═NO2)C3═CC═C(F)C═C3)═C(C(═C4)C(C)C)O |
| Example 780 | CC(═O)NC(C(═O)NCC2═C(C═1SC═C(C(═O)NC(C)C)N═1)C(═NO2)C3═CC═C(F)C═C3)CC4═CC═C(C═C4)O |
| Example 781 | ClC1═CC(Cl)═CC(═C1C(═O)NCC3═C(C═2SC═C(C(═O)NC(C)C)N═2)C(═NO3)C4═CC═C(F)C═C4)Cl |
| Example 782 | COC1═CC(═C(C═C1OC)[N+](═O)[O−])C(═O)NCC3═C(C═2SC═C(C(═O)NC(C)C)N═2)C(═NO3)C4═CC═C(F)C═C4 |
| Example 783 | O═C(C2═CC═C(OCC1═CC═CC═C1)C═C2)NCC4═C(C═3SC═C(C(═O)NC(C)C)N═3)C(═NO4)C5═CC═C(F)C═C5 |
| Example 784 | BrC═4C═CC═CC═4CCC(═O)NCC2═C(C═1SC═C(C(═O)NC(C)C)N═1)C(═NO2)C3═CC═C(F)C═C3 |
| Example 785 | O═C(CCC═1C═C(Br)C═CC═1)NCC3═C(C═2SC═C(C(═O)NC(C)C)N═2)C(═NO3)C4═CC═C(F)C═C4 |
| Example 786 | CC(OC(N4C(C(═O)NCC2═C(C═1SC═C(C(═O)NC(C)C)N═1)C(═NO2)C3═CC═C(F)C═C3)CCCC4)═O)(C)C |
| Example 787 | C5═CC═C(C═C5OC═1C═CC(═CC═1)C(═O)NCC3═C(C═2SC═C(C(═O)NC(C)C)N═2)C(═NO3)C4═CC═C(F)C═C4)O |
| Example 788 | CC(C(═O)NCC2═C(C═1 SC═C(C(═O)NC(C)C)N═1)C(═NO2)C3═CC═C(F)C═C3)C═5C═CC4═CC(═CC═C4C═5)OC |
| Example 789 | CC(C)(OC(N1CC(NCC1)C(═O)NCC3═C(C═2SC═C(C(═O)NC(C)C)N═2)C(═NO3)C4═CC═C(F)C═C4)═O)C |
| Example 790 | CC(C)(OC(N1CC(NCC1)C(═O)NCC3═C(C═2SC═C(C(═O)NC(C)C)N═2)C(═NO3)C4═CC═C(F)C═C4)═O)C |
| Example 791 | COC1═CC(═C(C═C1)Br)C(═O)NCC3═C(C═2SC═C(C(═O)NC(C)C)N═2)C(═NO3)C4═CC═C(F)C═C4 |
| Example 792 | CC(OC(═O)N1CC(CC1C(═O)NCC3═C(C═2SC═C(C(═O)NC(C)C)N═2)C(═NO3)C4═CC═C(F)C═C4)O)(C)C |
| Example 793 | CC(C)C(C(═O)NCC2═C(C═1SC═C(C(═O)NC(C)C)N═1)C(═NO2)C3═CC═C(F)C═C3)N(C(OC(C)(C)C)═O)C |
| Example 794 | FC(F)(C4═CC([N+]([O−])═O)═C(C(═O)NCC2═C(C═1SC═C(C(═O)NC(C)C)N═1)C(═NO2)C3═CC═C(F)C═C3)C═C4)F |
| Example 795 | BrC1═CC═C(Cl)C(═C1)C(═O)NCC3═C(C═2SC═C(C(═O)NC(C)C)N═2)C(═NO3)C4═CC═C(F)C═C4 |
| Example 796 | O═C(C═1C═C(Br)C(═CC═1)Cl)NCC3═C(C═2SC═C(C(═O)NC(C)C)N═2)C(═NO3)C4═CC═C(F)C═C4 |
| Example 797 | C═1C═C(C═CC═1O)C(═O)C2═CC═CC═C2C(═O)NCC4═C(C═3SC═C(C(═O)NC(C)C)N═3)C(═NO4)C5═CC═C(F)C═C5 |
| Example 798 | C1═CC(═CC═C1F)C(═O)C5═C(C(═O)NCC3═C(C═2SC═C(C(═O)NC(C)C)N═2)C(═NO3)C4═CC═C(F)C═C4)C═CC═C5 |
| Example 799 | O═C(C(C2═CC(═C(C1═CC═CC═C1)C═C2)F)C)NCC4═C(C═3SC═C(C(═O)NC(C)C)N═3)C(═NO4)C5═CC═C(F)C═C5 |
| Example 800 | C═1C═C(C═CC═1l)C(═O)NCC3═C(C═2SC═C(C(═O)NC(C)C)N═2)C(═NO3)C4═CC═C(F)C═C4 |
| Example 801 | IC1═CC═CC(═C1)C(═O)NCC3═C(C═2SC═C(C(═O)NC(C)C)N═2)C(═NO3)C4═CC═C(F)C═C4 |
| Example 802 | O═C(OCC1═CC═CC═C1)N2C(CCC2)C(═O)NCC4═C(C═3SC═C(C(═O)NC(C)C)N═3)C(═NO4)C5═CC═C(F)C═C5 |
| Example 803 | CC(CC)C(C(═O)NCC2═C(C═1SC═C(C(═O)NC(C)C)N═1)C(═NO2)C3═CC═C(F)C═C3)NC(OC(C)(C)C)═O |
| Example 804 | CC(C)(OC(NC(C(═O)NCC2═C(C═1SC═C(C(═O)NC(C)C)N═1)C(═NO2)C3═CC═C(F)C═C3)CC(C)C)═O)C |
| Example 805 | O═C(C1═CC═CC═C1)C═2C═C(C═CC═2)C(C)C(═O)NCC4═C(C═3SC═C(C(═O)NC(C)C)N═3)C(═NO4)C5═CC═C(F)C═C5 |
| Example 806 | CC(C)(OC(NC(C(═O)NCC2═C(C═1SC═C(C(═O)NC(C)C)N═1)C(═NO2)C3═CC═C(F)C═C3)CC4═CC═CC═C4)═O)C |
| Example 807 | CC(C)(OC(NC(C(═O)NCC2═C(C═1SC═C(C(═O)NC(C)C)N═1)C(═NO2)C3═CC═C(F)C═C3)CC4═NC═CC═C4)═O)C |
| Example 808 | CC(OC(═O)N2CC═1C═CC═CC═1CC2C(═O)NCC4═C(C═3SC═C(C(═O)NC(C)C)N═3)C(═NO4)C5═CC═C(F)C═C5)(C)C |
| Example 809 | C1═CC(═CC═C1O)CC(C(═O)NCC3═C(C═2SC═C(C(═O)NC(C)C)N═2)C(═NO3)C4═CC═C(F)C═C4)NC(OC(C)(C)C)═O |
| Example 810 | N1═C(SC═C1C(═O)N(C(C)C)[H])C2═C(CN)ON═C2C3═CC═C(F)C═C3 |

### EXAMPLES 811-1048

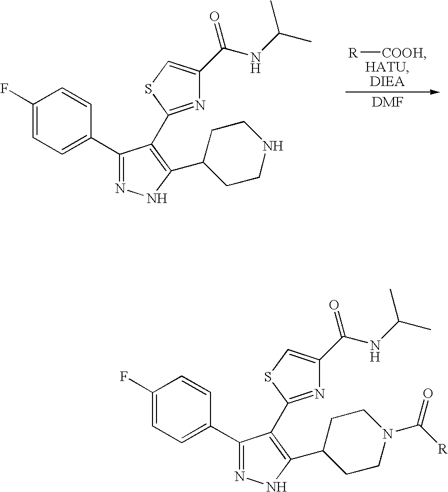

Starting piperidinyl pyrazole was prepared as described in Example 47. Where, R—COOH is a carboxylic acid selected to afford Examples 811-1048, which were prepared by General Procedure 1.

| | |
|---|---|
| Example 811 | [H]N2N═C(C1═CC═C(C═C1)F)C(═C2C3CCNCC3)C4═NC(═CS4)C(NC(C)C)═O |
| Example 812 | [H]N2N═C(C1═CC═C(F)C═C1)C(═C2C3CCN(CC3)C(═O)C)C4═NC(C(NC(C)C)═O)═CS4 |
| Example 813 | [H]N2N═C(C1═CC═C(F)C═C1)C(═C2C3CCN(CC3)C(C═O)═O)C4═NC(C(NC(C)C)═O)═CS4 |
| Example 814 | [H]N2N═C(C1═CC═C(F)C═C1)C(═C2C3CCN(CC3)C(CC#N)═O)C4═NC(C(NC(C)C)═O)═CS4 |
| Example 815 | [H]N2N═C(C1═CC═C(F)C═C1)C(═C2C3CCN(CC3)C(C(C)O)═O)C4═NC(C(NC(C)C)═O)═CS4 |
| Example 816 | [H]N2N═C(C1═CC═C(F)C═C1)C(═C2C3CCN(CC3)C(C(CC)═O)═O)C4═NC(C(NC(C)C)═O)═CS4 |
| Example 817 | [H]N2N═C(C1═CC═C(F)C═C1)C(═C2C3CCN(CC3)C(C(C)(C)C)═O)C4═NC(C(NC(C)C)═O)═CS4 |
| Example 818 | [H]N2N═C(C1═CC═C(F)C═C1)C(═C2C3CCN(CC3)C(CC(C)C)═O)C4═NC(C(NC(C)C)═O)═CS4 |
| Example 819 | [H]N5C(C(═O)N4CCC(C═2N(N═C(C1═CC═C(F)C═C1)C═2C3═NC(C(NC(C)C)═O)═CS3)[H])CC4)═CN═C5 |
| Example 820 | [H]N1C(═NC═C1)C(═O)N5CCC(C═3N(N═C(C2═CC═C(F)C═C2)C═3C4═NC(C(NC(C)C)═O)═CS4)[H])CC5 |
| Example 821 | [H]N2N═C(C1═CC═C(F)C═C1)C(═C2C3CCN(CC3)C(C4═CCCC4)═O)C5═NC(C(NC(C)C)═O)═CS5 |
| Example 822 | [H]N2N═C(C1═CC═C(F)C═C1)C(═C2C3CCN(CC3)C(C(F)(F)F)═O)C4═NC(C(NC(C)C)═O)═CS4 |
| Example 823 | [H]N2N═C(C1═CC═C(F)C═C1)C(═C2C3CCN(CC3)C(═O)C4OCCC4)C5═NC(C(NC(C)C)═O)═CS5 |
| Example 824 | [H]N2N═C(C1═CC═C(F)C═C1)C(═C2C3CCN(CC3)C(CNC(C)═O)═O)C4═NC(C(NC(C)C)═O)═CS4 |
| Example 825 | [H]N2N═C(C1═CC═C(F)C═C1)C(═C2C3CCN(CC3)C═(O)C═4C═CC═CC═4)C5═NC(C(NC(C)C)═O)═CS5 |
| Example 826 | [H]N2N═C(C1═CC═C(F)C═C1)C(═C2C3CCN(CC3)C(═O)C═4C═NC═CC═4)C5═NC(C(NC(C)C)═O)═CS5 |
| Example 827 | [H]N2N═C(C1═CC═C(F)C═C1)C(═C2C3CCN(CC3)C(C4═CC═NC═C4)═O)C5═NC(C(NC(C)C)═O)═CS5 |
| Example 828 | [H]N2N═C(C1═CC═C(F)C═C1)C(═C2C3CCN(CC3)C(C4═CC═CC═N4)═O)C5═NC(C(NC(C)C)═O)═CS5 |
| Example 829 | [H]N2N═C(C1═CC═C(F)C═C1)C(═C2C3CCN(CC3)C(C(CCl)O)═O)C4═NC(C(NC(C)C)═O)═CS4 |
| Example 830 | [H]N1N═C(C═C1C)C(═O)N5CCC(C═3N(N═C(C2═CC═C(F)C═C2)C═3C4═NC(C(NC(C)C)═O)═CS4)[H])CC5 |
| Example 831 | [H]N2N═C(C1═CC═C(F)C═C1)C(═C2C3CCN(CC3)C(C═4N═CN(C═4)C)═O)C5═NC(C(NC(C)C)═O)═CS5 |
| Example 832 | [H]N2N═C(C1═CC═C(F)C═C1)C(═C2C3CCN(CC3)C(C4CCCCC4)═O)C5═NC(C(NC(C)C)═O)═CS5 |
| Example 833 | [H]N2N═C(C1═CC═C(F)C═C1)C(═C2C3CCN(CC3)C(═O)C4═CSC═N4)C5═NC(C(NC(C)C)═O)═CS5 |
| Example 834 | [H]N2N═C(C1═CC═C(F)C═C1)C(═C2C3CCN(CC3)C(CC(OCC)═O)═O)C4═NC(C(NC(C)C)═O)═CS4 |
| Example 835 | [H]N2N═C(C1═CC═C(F)C═C1)C(═C2C3CCN(CC3)C(═O)C═4C═C(C═CC═4)C)C5═NC(C(NC(C)C)═O)═CS5 |
| Example 836 | [H]N2N═C(C1═CC═C(F)C═C1)C(═C2C3CCN(CC3)C(C4═CC═CC═C4C)═O)C5═NC(C(NC(C)C)═O)═CS5 |
| Example 837 | [H]N2N═C(C1═CC═C(F)C═C1)C(═C2C3CCN(CC3)C(C4═CC═C(C)C═C4)═O)C5═NC(C(NC(C)C)═O)═CS5 |
| Example 838 | [H]N2N═C(C1═CC═C(F)C═C1)C(═C2C3CCN(CC3)C(C═4C═C(C═CC═4)O)═O)C5═NC(C(NC(C)C)═O)═CS5 |
| Example 839 | [H]N2N═C(C1═CC═C(F)C═C1)C(═C2C3CCN(CC3)C(═O)C═4C(═CC═CC═4)O)C5═NC(C(NC(C)C)═O)═CS5 |
| Example 840 | [H]N2N═C(C1═CC═C(F)C═C1)C(═C2C3CCN(CC3)C(C═4C═CC(F)═CC═4)═O)C5═NC(C(NC(C)C)═O)═CS5 |
| Example 841 | [H]N2N═C(C1═CC═C(F)C═C1)C(═C2C3CCN(CC3)C(C═4C═C(F)C═CC═4)═O)C5═NC(C(NC(C)C)═O)═CS5 |
| Example 842 | [H]N2N═C(C1═CC═C(F)C═C1)C(═C2C3CCN(CC3)C(═O)CCCCCCC)C4═NC(C(NC(C)C)═O)═CS4 |
| Example 843 | [H]N2N═C(C1═CC═C(F)C═C1)C(═C2C3CCN(CC3)C(C4═CC═C(C#N)C═C4)═O)C5═NC(C(NC(C)C)═O)═CS5 |
| Example 844 | [H]N2N═C(C1═CC═C(F)C═C1)C(═C2C3CCN(CC3)C(C4═CC═CC(C#N)═C4)═O)C5═NC(C(NC(C)C)═O)═CS5 |
| Example 845 | [H]N2N═C(C1═CC═C(F)C═C1)C(═C2C3CCN(CC3)C(═O)/C═C/C═4C═CC═CC═4)C5═NC(C(NC(C)C)═O)═CS5 |
| Example 846 | [H]N2N═C(C1═CC═C(F)C═C1)C(═C2C3CCN(CC3)C(C4═CC═CC═C4C═O)═O)C5═NC(C(NC(C)C)═O)═CS5 |
| Example 847 | [H]N2N═C(C1═CC═C(F)C═C1)C(═C2C3CCN(CC3)C(C4═CC═C(C═O)C═C4)═O)C5═NC(C(NC(C)C)═O)═CS5 |
| Example 848 | [H]N2N═C(C1═CC═C(F)C═C1)C(═C2C3CCN(CC3)C(C═4C(═CC(C)═CC═4)C)═O)C5═NC(C(NC(C)C)═O)═CS5 |
| Example 849 | [H]N2N═C(C1═CC═C(F)C═C1)C(═C2C3CCN(CC3)C(CC═4C(C)═CC═CC═4)═O)C5═NC(C(NC(C)C)═O)═CS5 |
| Example 850 | [H]N2N═C(C1═CC═C(F)C═C1)C(═C2C3CCN(CC3)C(═O)C═4C(C)═C(C)C═CC═4)C5═NC(C(NC(C)C)═O)═CS5 |
| Example 851 | [H]N2N═C(C1═CC═C(F)C═C1)C(═C2C3CCN(CC3)C(C═4C═C(C)C═CC═4C)═O)C5═NC(C(NC(C)C)═O)═CS5 |
| Example 852 | [H]N2N═C(C1═CC═C(F)C═C1)C(═C2C3CCN(CC3)C(C4═CC(C)═CC(═C4)C)═O)C5═NC(C(NC(C)C)═O)═CS5 |
| Example 853 | [H]N2N═C(C1═CC═C(F)C═C1)C(═C2C3CCN(CC3)C(═O)C4═CC═C(C)C(═C4)C)C5═NC(C(NC(C)C)═O)═CS5 |
| Example 854 | [H]N2N═C(C1═CC═C(F)C═C1)C(═C2C3CCN(CC3)C(C═4C═CC(CC)═CC═4)═O)C5═NC(C(NC(C)C)═O)═CS5 |
| Example 855 | [H]N2N═C(C1═CC═C(F)C═C1)C(═C2C3CCN(CC3)C(═O)C4═CC═C(C═C4)OC)C5═NC(C(NC(C)C)═O)═CS5 |
| Example 856 | [H]N2N═C(C1═CC═C(F)C═C1)C(═C2C3CCN(CC3)C(C4═C(C(═CC═C4)C)O)═O)C5═NC(C(NC(C)C)═O)═CS5 |
| Example 857 | [H]N2N═C(C1═CC═C(F)C═C1)C(═C2C3CCN(CC3)C(CC4═C(C═CC═C4)O)═O)C5═NC(C(NC(C)C)═O)═CS5 |
| Example 858 | [H]N2N═C(C1═CC═C(F)C═C1)C(═C2C3CCN(CC3)C(CC═4C═C(O)C═CC═4)═O)C5═NC(C(NC(C)C)═O)═CS5 |
| Example 859 | [H]N2N═C(C1═CC═C(F)C═C1)C(═C2C3CCN(CC3)C(C═4C═C(C)C═CC═4O)═O)C5═NC(C(NC(C)C)═O)═CS5 |
| Example 860 | [H]N2N═C(C1═CC═C(F)C═C1)C(═C2C3CCN(CC3)C(C═4C(═CC═C(O)C═4)O)═O)C5═NC(C(NC(C)C)═O)═CS5 |
| Example 861 | [H]N2N═C(C1═CC═C(F)C═C1)C(═C2C3CCN(CC3)C(C═4C(═C(O)C═CC═4)O)═O)C5═NC(C(NC(C)C)═O)═CS5 |
| Example 862 | [H]N2N═C(C1═CC═C(F)C═C1)C(═C2C3CCN(CC3)C(═O)C═4C(═CC(═CC═4)O)O)C5═NC(C(NC(C)C)═O)═CS5 |
| Example 863 | [H]N2N═C(C1═CC═C(F)C═C1)C(═C2C3CCN(CC3)C(C═4C═C(C(C)═CC═4)F)═O)C5═NC(C(NC(C)C)═O)═CS5 |
| Example 864 | [H]N2N═C(C1═CC═C(F)C═C1)C(═C2C3CCN(CC3)C(═O)C═4C(═CC═C(C═4)F)C)C5═NC(C(NC(C)C)═O)═CS5 |
| Example 865 | [H]N2N═C(C1═CC═C(F)C═C1)C(═C2C3CCN(CC3)C(C═4C(═CC═CC═4)Cl)═O)C5═NC(C(NC(C)C)═O)═CS5 |
| Example 866 | [H]N2N═C(C1═CC═C(F)C═C1)C(═C2C3CCN(CC3)C(C4═CN═C(Cl)C═C4)═O)C5═NC(C(NC(C)C)═O)═CS5 |
| Example 867 | [H]N2N═C(C1═CC═C(F)C═C1)C(═C2C3CCN(CC3)C(C4═C(N═CC═C4)Cl)═O)C5═NC(C(NC(C)C)═O)═CS5 |
| Example 868 | [H]N2N═C(C1═CC═C(F)C═C1)C(═C2C3CCN(CC3)C(C4═CC(═NC═C4)Cl)═O)C5═NC(C(NC(C)C)═O)═CS5 |
| Example 869 | [H]N2N═C(C1═CC═C(F)C═C1)C(═C2C3CCN(CC3)C(C4═NC═CC(═C4)Cl)═O)C5═NC(C(NC(C)C)═O)═CS5 |
| Example 870 | [H]N2N═C(C1═CC═C(F)C═C1)C(═C2C3CCN(CC3)C(C4═C(C═NC═C4)Cl)═O)C5═NC(C(NC(C)C)═O)═CS5 |
| Example 871 | [H]N2N═C(C1═CC═C(F)C═C1)C(═C2C3CCN(CC3)C(C4═C(C═CC═C4F)F)═O)C5═NC(C(NC(C)C)═O)═CS5 |
| Example 872 | [H]N2N═C(C1═CC═C(F)C═C1)C(═C2C3CCN(CC3)C(C4═CC═CC(F)═C4F)═O)C5═NC(C(NC(C)C)═O)═CS5 |
| Example 873 | [H]N2N═C(C1═CC═C(F)C═C1)C(═C2C3CCN(CC3)C(C4═CC═C(F)C═C4F)═O)C5═NC(C(NC(C)C)═O)═CS5 |
| Example 874 | [H]N2N═C(C1═CC═C(F)C═C1)C(═C2C3CCN(CC3)C(C═4C═C(C═C(F)C═4)F)═O)C5═NC(C(NC(C)C)═O)═CS5 |
| Example 875 | [H]N2N═C(C1═CC═C(F)C═C1)C(═C2C3CCN(CC3)C(C4═CC═C(F)C(═C4)F)═O)C5═NC(C(NC(C)C)═O)═CS5 |
| Example 876 | [H]N2N═C(C1═CC═C(F)C═C1)C(═C2C3CCN(CC3)C(CC(CC(C)(C)C)C)═O)C4═NC(C(NC(C)C)═O)═CS4 |
| Example 877 | [H]N2N═C(C1═CC═C(F)C═C1)C(═C2C3CCN(CC3)C(═O)C═5OC4═CC═CC═C4C═5)C6═NC(C(NC(C)C)═O)═CS6 |
| Example 878 | [H]N2N═C(C1═CC═C(F)C═C1)C(═C2C3CCN(CC3)C(C5CCC4═CC═CC═C45)═O)C6═NC(C(NC(C)C)═O)═CS6 |
| Example 879 | [H]N2N═C(C1═CC═C(F)C═C1)C(═C2C3CCN(CC3)C(═O)C(Cl)(Cl)Cl)C4═NC(C(NC(C)C)═O)═CS4 |
| Example 880 | [H]N2N═C(C1═CC═C(F)C═C1)C(═C2C3CCN(CC3)C(/C═C/C═4C(O)═CC═CC═4)═O)C5═NC(C(NC(C)C)═O)═CS5 |
| Example 881 | [H]N2N═C(C1═CC═C(F)C═C1)C(═C2C3CCN(CC3)C(C4═CC═C(C(C)═O)C═C4)═O)C5═NC(C(NC(C)C)═O)═CS5 |
| Example 882 | [H]N2N═C(C1═CC═C(F)C═C1)C(═C2C3CCN(CC3)C(/C═C/C4═CC(O)═CC═C4)═O)C5═NC(C(NC(C)C)═O)═CS5 |
| Example 883 | [H]N2N═C(C1═CC═C(F)C═C1)C(═C2C3CCN(CC3)C(C═4C═CC═C(C(═O)C)C═4)═O)C5═NC(C(NC(C)C)═O)═CS5 |
| Example 884 | [H]N2N═C(C1═CC═C(F)C═C1)C(═C2C3CCN(CC3)C(C4═CC═CC═C4C(═O)C)═O)C5═NC(C(NC(C)C)═O)═CS5 |
| Example 885 | [H]N2N═C(C1═CC═C(F)C═C1)C(═C2C3CCN(CC3)C(═O)C4═CC═C(C(C)C)C═C4)C5═NC(C(NC(C)C)═O)═CS5 |
| Example 886 | [H]N2N═C(C1═CC═C(F)C═C1)C(═C2C3CCN(CC3)C(CCC4═C(C═CC═C4)C)═O)C5═NC(C(NC(C)C)═O)═CS5 |
| Example 887 | [H]N2N═C(C1═CC═C(F)C═C1)C(═C2C3CCN(CC3)C(CCC═4C═C(C)C═CC═4)═O)C5═NC(C(NC(C)C)═O)═CS5 |
| Example 888 | [H]N2N═C(C1═CC═C(F)C═C1)C(═C2C3CCN(CC3)C(C(CC)C4═CC═CC═C4)═O)C5═NC(C(NC(C)C)═O)═CS5 |
| Example 889 | [H]N2N═C(C1═CC═C(F)C═C1)C(═C2C3CCN(CC3)C(C4═C(C═C(C)═C4C)C)═O)C5═NC(C(NC(C)C)═O)═CS5 |
| Example 890 | [H]N2N═C(C1═CC═C(F)C═C1)C(═C2C3CCN(CC3)C(C4═CC═C(CCC)C═C4)═O)C5═NC(C(NC(C)C)═O)═CS5 |
| Example 891 | [H]N2N═C(C1═CC═C(F)C═C1)C(═C2C3CCN(CC3)C(C═4C═CC═5OCOC═5C═4)═O)C6═NC(C(NC(C)C)═O)═CS6 |
| Example 892 | [H]N2N═C(C1═CC═C(F)C═C1)C(═C2C3CCCN(CC3)C(/C═C/C═4C(F)═CC═CC═4)═O)C5═NC(C(NC(C)C)═O)═CS5 |
| Example 893 | [H]N2N═C(C1═CC═C(F)C═C1)C(═C2C3CCN(CC3)C(C═4C(OCC)═CC═CC═4)═O)C5═NC(C(NC(C)C)═O)═CS5 |
| Example 894 | [H]N2N═C(C1═CC═C(F)C═C1)C(═C2C3CCN(CC3)C(COC4═C(C═CC═C4)C)═O)C5═NC(C(NC(C)C)═O)═CS5 |
| Example 895 | [H]N2N═C(C1═CC═C(F)C═C1)C(═C2C3CCN(CC3)C(CCC4═CC═CC═C4O)═O)C5═NC(C(NC(C)C)═O)═CS5 |
| Example 896 | [H]N2N═C(C1═CC═C(F)C═C1)C(═C2C3CCN(CC3)C(CC4═C(OC)C═CC═C4)═O)C5═NC(C(NC(C)C)═O)═CS5 |
| Example 897 | [H]N2N═C(C1═CC═C(F)C═C1)C(═C2C3CCN(CC3)C(C(C═4C═CC(O)═CC═4)C)═O)C5═NC(C(NC(C)C)═O)═CS5 |
| Example 898 | [H]N2N═C(C1═CC═C(F)C═C1)C(═C2C3CCN(CC3)C(C4═CC═CC═C4[N+]([O−])═O)═O)C5═NC(C(NC(C)C)═O)═CS5 |
| Example 899 | [H]N2N═C(C1═CC═C(F)C═C1)C(═C2C3CCN(CC3)C(C4═CC═CC([N+]([O−])═O)═C4)═O)C5═NC(C(NC(C)C)═O)═CS5 |
| Example 900 | [H]N2N═C(C1═CC═C(F)C═C1)C(═C2C3CCN(CC3)C(C═4C(O)═CC═CC═4OC)═O)C5═NC(C(NC(C)C)═O)═CS5 |
| Example 901 | [H]N2N═C(C1═CC═C(F)C═C1)C(═C2C3CCN(CC3)C(C═4C═C═(OC)C═CC═4O)═O)C5═NC(C(NC(C)C)═O)═CS5 |
| Example 902 | [H]N2N═C(C1═CC═C(F)C═C1)C(═C2C3CCN(CC3)C(C4═C(C═C(OC)C═C4)O)═O)C5═NC(C(NC(C)C)═O)═CS5 |
| Example 903 | [H]N2N═C(C1═CC═C(F)C═C1)C(═C2C3CCN(CC3)C(C4═CC(O)═C(OC)C═C4)═O)C5═NC(C(NC(C)C)═O)═CS5 |
| Example 904 | [H]N2N═C(C1═CC═C(F)C═C1)C(═C2C3CCN(CC3)C(C═4C═C(C(O)═C(C═4)O)O)═O)C5═NC(C(NC(C)C)═O)═CS5 |
| Example 905 | [H]N2N═C(C1═CC═C(F)C═C1)C(═C2C3CCN(CC3)C(═O)CC═4C═C(C(═CC═4)O)F)C5═NC(C(NC(C)C)═O)═CS5 |
| Example 906 | [H]N2N═C(C1═CC═C(F)C═C1)C(═C2C3CCN(CC3)C(C═4C═C(N═C(C═4)Cl)C)═O)C5═NC(C(NC(C)C)═O)═CS5 |
| Example 907 | [H]N2N═C(C1═CC═C(F)C═C1)C(═C2C3CCN(CC3)C(CC4═C(C═CC═C4F)F)═O)C5═NC(C(NC(C)C)═O)═CS5 |
| Example 908 | [H]N2N═C(C1═CC═C(F)C═C1)C(═C2C3CCN(CC3)C(C5═CC═CC4═CC═CC═C45)═O)C6═NC(C(NC(C)C)═O)═CS6 |
| Example 909 | [H]N2N═C(C1═CC═C(F)C═C1)C(═C2C3CCN(CC3)C(C4═CC═C(Cl)C═C4 O)═O)C5═NC(C(NC(C)C)═O)═CS5 |
| Example 910 | [H]N2N═C(C1═CC═C(F)C═C1)C(═C2C3CCN(CC3)C(═O)C═4C(═CC═C(C═4)Cl)O)C5═NC(C(NC(C)C)═O)═CS5 |
| Example 911 | [H]N2N═C(C1═CC═C(F)C═C1)C(═C2C3CCN(CC3)C(C4═CC═5C═CC═NC═5C═C4)═O)C6═NC(C(NC(C)C)═O)═CS6 |
| Example 912 | [H]N2N═C(C1═CC═C(F)C═C1)C(═C2C3CCN(CC3)C(═O)C4═CN═C(C(═C4)Cl)O)C5═NC(C(NC(C)C)═O)═CS5 |
| Example 913 | [H]N2N═C(C1═CC═C(F)C═C1)C(═C2C3CCN(CC3)C(C4═CC═C(F)C═C4Cl)═O)C5═NC(C(NC(C)C)═O)═CS5 |
| Example 914 | [H]N2N═C(C1═CC═C(F)C═C1)C(═C2C3CCN(CC3)C(C4═C(C═CC═C4Cl)F)═O)C5═NC(C(NC(C)C)═O)═CS5 |
| Example 915 | [H]N2N═C(C1═CC═C(F)C═C1)C(═C2C3CCN(CC3)C(CNC(OC(C)(C)C)═O)═O)C4═NC(C(NC(C)C)═O)═CS4 |
| Example 916 | [H]N2N═C(C1═CC═C(F)C═C1)C(═C2C3CCN(CC3)C(C═4OC5═CC═CC═C5C═4C)═O)C6═NC(C(NC(C)C)═O)═CS6 |
| Example 917 | [H]N2N═C(C1═CC═C(F)C═C1)C(═C2C3CCN(CC3)C(CC5CC4═C(C═CC═C4)C5)═O)C6═NC(C(NC(C)C)═O)═CS6 |
| Example 918 | [H]N2N═C(C1═CC═C(F)C═C1)C(═C2C3CCN(CC3)C(═O)C5CC4═C(C═C C═C4)CC5)C6═NC(C(NC(C)C)═O)═CS6 |
| Example 919 | [H]N2N═C(C1═CC═C(F)C═C1)C(═C2C3CCN(CC3)C(/C═C/C═4C═CC═C C═4OC)═O)C5═NC(C(N C(C)C)═O)═CS5 |
| Example 920 | [H]N2N═C(C1═CC═C(F)C═C1)C(═C2C3CCN(CC3)C(C4═CSC5═C4C═CC═C5)═O)C6═NC(C(NC(C)C)═O)═CS6 |
| Example 921 | [H]N2N═C(C1═CC═C(F)C═C1)C(═C2C3CCN(CC3)C(C═4SC5═CC═CC═C 5C═4)═O)C6═NC(C(NC(C)C)═O)═CS6 |
| Example 922 | [H]N2N═C(C1═CC═C(F)C═C1)C(═C2C3CCN(CC3)C(C4═CC═C(CCCC)C═C4)═O)C5═NC(C(NC(C)C)═O)═CS5 |
| Example 923 | [H]N2N═C(C1═CC═C(F)C═C1)C(═C2C3CCN(CC3)C(C4═CC═C(C(C)(C)C)C═C4)═O)C5═NC(C(NC(C)C)═O)═CS5 |
| Example 924 | [H]N2N═C(C1═CC═C(F)C═C1)C(═C2C3CCN(CC3)C(C4═CC═C(N(C═O)C)C═C4)═O)C5═NC(C(NC(C)C)═O)═CS5 |
| Example 925 | [H]N2N═C(C1═CC═C(F)C═C1)C(═C2C3CCN(CC3)C(C═4C═C(NC(C)═O)C═CC═4)═O)C5═NC(C(NC(C)C)═O)═CS5 |
| Example 926 | [H]N2N═C(C1═CC═C(F)C═C1)C(═C2C3CCN(CC3)C(COC═4C(C═O)═CC═CC═4)═O)C5═NC(C(N C(C)C)═O)═CS5 |
| Example 927 | [H]N2N═C(C1═CC═C(F)C═C1)C(═C2C3CCN(CC3)C(C4═CC═C(OC(═O)C)C═C4)═O)C5═NC(C(NC(C)C)═O)═CS5 |
| Example 928 | [H]N2N═C(C1═CC═C(F)C═C1)C(═C2C3CCN(CC3)C(C4═CC═C(C(OC)═O)C═C4)═O)C5═NC(C(NC(C)C)═O)═CS5 |
| Example 929 | [H]N2N═C(C1═CC═C(F)C═C1)C(═C2C3CCN(CC3)C(═O)C4═CC═CC(OC(═O)C)═C4)C5═NC(C(NC(C)C)═O)═CS5 |
| Example 930 | [H]N2N═C(C1═CC═C(F)C═C1)C(═C2C3CCN(CC3)C(C4═CC═CC═C4OC(═O)C)═O)C5═NC(C(NC(C)C)═O)═CS5 |
| Example 931 | [H]N2N═C(C1═CC═C(F)C═C1)C(═C2C3CCN(CC3)C(CC4═CC═5OCOC═5C═C4)═O)C6═NC(C(N C(C)C)═O)═CS6 |
| Example 932 | [H]N2N═C(C1═CC═C(F)C═C1)C(═C2C3CCN(CC3)C(C4═CC═C(OCCC)C═C4)═O)C5═NC(C(NC(C)C)═O)═CS5 |
| Example 933 | [H]N2N═C(C1═CC═C(F)C═C1)C(═C2C3CCN(CC3)C(C4═CC═C(OC(C)C)C═C4)═O)C5═NC(C(NC(C)C)═O)═CS5 |
| Example 934 | [H]N2N═C(C1═CC═C(F)C═C1)C(═C2C3CCN(CC3)C(C4═C([N+](═O)[O−])C═CC═C4C)═O)C5═N C(C(NC(C)C)═O)═CS5 |
| Example 935 | [H]N2N═C(C1═CC═C(F)C═C1)C(═C2C3CCN(CC3)C(C4═CC═CC(═C4[N+]([O−])═O)C)═O)C5═NC(C(NC(C)C)═O)═CS5 |
| Example 936 | [H]N2N═C(C1═CC═C(F)C═C1)C(═C2C3CCN(CC3)C(C4═CC(═CC═C4[N+](═O)[O−])C)═O)C5═NC(C(NC(C)C)═O)═CS5 |
| Example 934 | [H]N2N═C(C1═CC═C(F)C═C1)C(═C2C3CCN(CC3)C(═O)C═4C(═C(C═CC═4)[N+]([O−])═O)C)C5═NC(C(NC(C)C)═O)═CS5 |
| Example 938 | [H]N2N═C(C1═CC═C(F)C═C1)C(═C2C3CCN(CC3)C(C═4C═C(OC)C═CC═4OC)═O)C5═NC(C(NC(C)C)═O)═CS5 |
| Example 939 | [H]N2N═C(C1═CC═C(F)C═C1)C(═C2C3CCN(CC3)C(C═4C(═CC(OC)═CC═4)OC)═O)C5═NC(C(NC(C)C)═O)═CS5 |
| Example 940 | [H]N2N═C(C1═CC═C(F)C═C1)C(═C2C3CCN(CC3)C(C4═C(OC)C═CC═C4OC)═O)C5═NC(C(NC(C)C)═O)═CS5 |
| Example 941 | [H]N2N═C(C1═CC═C(F)C═C1)C(═C2C3CCN(CC3)C(C4═CC(OC)═CC(═C4)OC)═O)C5═NC(C(NC(C)C)═O)═CS5 |
| Example 942 | [H]N2N═C(C1═CC═C(F)C═C1)C(═C2C3CCN(CC3)C(C4═C(C(OC)═CC═C4)OC)═O)C5═NC(C(NC(C)C)═O)═CS5 |
| Example 943 | [H]N2N═C(C1═CC═C(F)C═C1)C(═C2C3CCN(CC3)C(═O)CSC4═CC═C(C═C4)C)C5═NC(C(NC(C)C)═O)═CS5 |
| Example 944 | [H]N2N═C(C1═CC═C(F)C═C1)C(═C2C3CCN(CC3)C(/C═C/C═4C═CC(Cl)═CC═4)═O)C5═NC(C(N C(C)C)═O)═CS5 |
| Example 945 | [H]N2N═C(C1═CC═C(F)C═C1)C(═C2C3CCN(CC3)C(/C═C/C═4C(Cl)═CC═CC═4)═O)C5═NC(C(N C(C)C)═O)═CS5 |
| Example 946 | [H]N2N═C(C1═CC═C(F)C═C1)C(═C2C3CCN(CC3)C(C═4C═C(O)C(═CC═4)[N+](═O)[O−])═O)C5═NC(C(NC(C)C)═O)═CS5 |
| Example 947 | [H]N2N═C(C1═CC═C(F)C═C1)C(═C2C3CCN(CC3)C(C4═CC(O)═CC═C4 [N+]([O−])═O)═O)C5═NC(C(NC(C)C)═O)═CS5 |
| Example 948 | [H]N2N═C(C1═CC═C(F)C═C1)C(═C2C3CCN(CC3)C(═O)C4═CC([N+](═O)[O−])═CC═C4O)C5═NC(C(NC(C)C)═O)═CS5 |
| Example 949 | [H]N2N═C(C1═CC═C(F)C═C1)C(═C2C3CCN(CC3)C(C═4C(═CC═C([N+]([O−])═O)C═4)F)═O)C5═NC(C(NC(C)C)═O)═CS5 |
| Example 950 | [H]N2N═C(C1═CC═C(F)C═C1)C(═C2C3CCN(CC3)C(C4═CC(═CC═C4[N+]([O−])═O)F)═O)C5═NC(C(NC(C)C)═O)═CS5 |
| Example 951 | [H]N2N═C(C1═CC═C(F)C═C1)C(═C2C3CCN(CC3)C(C4═CC═C(C([N+]([O−])═O)═C4)F)═O)C5═NC(C(NC(C)C)═O)═CS5 |
| Example 952 | [H]N2N═C(C1═CC═C(F)C═C1)C(═C2C3CCN(CC3)C(C═4C(═CC═C(Cl)C═4)OC)═O)C5═NC(C(NC(C)C)═O)═CS5 |
| Example 953 | [H]N2N═C(C1═CC═C(F)C═C1)C(═C2C3CCN(CC3)C(C4═CC═C(C═C4OC)Cl)═O)C5═NC(C(NC(C)C)═O)═CS5 |
| Example 954 | [H]N2N═C(C1═CC═C(F)C═C1)C(═C2C3CCN(CC3)C(CC4═CC(═C(O)C═C4)Cl)═O)C5═NC(C(NC(C)C)═O)═CS5 |
| Example 955 | [H]N2N═C(C1═CC═C(F)C═C1)C(═C2C3CCN(CC3)C(C═4C═C(N═C(C═4)Cl)OC)═O)C5═NC(C(NC(C)C)═O)═CS5 |
| Example 956 | [H]N2N═C(C1═CC═C(F)C═C1)C(═C2C3CCN(CC3)C(C4═CC═5C(C═C4)═CC(═CC═5)O)═O)C6═NC(C(NC(C)C)═O)═CS6 |
| Example 957 | [H]N2N═C(C1═CC═C(F)C═C1)C(═C2C3CCN(CC3)C(C═4C5═CC═CC═C5C═CC═4O)═O)C6═NC(C(NC(C)C)═O)═CS6 |
| Example 958 | [H]N2N═C(C1═CC═C(F)C═C1)C(═C2C3CCN(CC3)C(C4═CC5═CC═CC═C5C═C4O)═O)C6═NC(C(NC(C)C)═O)═CS6 |
| Example 959 | [H]N2N═C(C1═CC═C(F)C═C1)C(═C2C3CCN(CC3)C(C═5C(═C4C(C═CC═C4)═CC═5)O)═O)C6═NC(C(NC(C)C)═O)═CS6 |
| Example 960 | [H]N2N═C(C1═CC═C(F)C═C1)C(═C2C3CCN(CC3)C(C═4N═C5C(═CC═CC5═CC═4)O)═O)C6═NC(C(NC(C)C)═O)═CS6 |
| Example 961 | [H]N2N═C(C1═CC═C(F)C═C1)C(═C2C3CCN(CC3)C(C(NC(OC(C)(C)C)═O)C)═O)C4═NC(C(NC(C)C)═O)═CS4 |
| Example 962 | [H]N2N═C(C1═CC═C(F)C═C1)C(═C2C3CCN(CC3)C(C(NC(OC(C)(C)C)═O)C)═O)C4═NC(C(NC(C)C)═O)═CS4 |
| Example 963 | [H]N2N═C(C1═CC═C(F)C═C1)C(═C2C3CCN(CC3)C(C═4C(═CC═CC═4)C(F)(F)F)═O)C5═NC(C(NC(C)C)═O)═CS5 |
| Example 964 | [H]N2C1═CC═CC═C1N═C2CCC(═O)N6CCC(C═4N(N═C(C3═CC═C(F)C═C3)C═4C5═NC(C(NC(C)C)═O)═CS5)[H])CC6 |
| Example 965 | [H]N2N═C(C1═CC═C(F)C═C1)C(═C2C3CCN(CC3)C(C═4C(═C(Cl)C═CC═4)Cl)═O)C5═NC(C(NC(C)C)═O)═CS5 |
| Example 966 | [H]N2N═C(C1═CC═C(F)C═C1)C(═C2C3CCN(CC3)C(C═4C(═CC(Cl)═CC═4)Cl)═O)C5═NC(C(NC(C)C)═O)═CS5 |
| Example 967 | [H]N2N═C(C1═CC═C(F)C═C1)C(═C2C3CCN(CC3)C(═O)C═4C(═CC═C(C═4)Cl)Cl)C5═NC(C(NC(C)C)═O)═CS5 |
| Example 968 | [H]N2N═C(C1═CC═C(F)C═C1)C(═C2C3CCN(CC3)C(C4═C(C(═C(C(═C4)F)F)O)F)═O)C5═NC(C(NC(C)C)═O)═CS5 |
| Example 969 | [H]N2N═C(C1═CC═C(F)C═C1)C(═C2C3CCN(CC3)C(/C═C/C4═CC═5OCOC═5C═C4)═O)C6═NC(C(NC(C)C)═O)═CS6 |
| Example 970 | [H]N2N═C(C1═CC═C(F)C═C1)C(═C2C3CCN(CC3)C(CCC(C4═CC═C(C)C═C4)═O)═O)C5═NC(C(NC(C)C)═O)═CS5 |
| Example 971 | [H]N2N═C(C1═CC═C(F)C═C1)C(═C2C3CCN(CC3)C(C4═CC═C(CCCCC)C═C4)═O)C5═NC(C(NC(C)C)═O)═CS5 |
| Example 972 | [H]N2N═C(C1═CC═C(F)C═C1)C(═C2C3CCN(CC3)C(C4═CC(═C(F)C═C4Cl)F)═O)C5═NC(C(NC(C)C)═O)═CS5 |
| Example 973 | [H]N2N═C(C1═CC═C(F)C═C1)C(═C2C3CCN(CC3)C(C═4C(═CC(Cl)═C(C═4)F)F)═O)C5═NC(C(NC(C)C)═O)═CS5 |
| Example 974 | [H]N2N═C(C1═CC═C(F)C═C1)C(═C2C3CCN(CC3)C(C4═CC═C(N(CC)CC)C═C4)═O)C5═NC(C(NC(C)C)═O)═CS5 |
| Example 975 | [H]N2N═C(C1═CC═C(F)C═C1)C(═C2C3CCN(CC3)C(═O)/C═C/C═4C═C(C(═CC═4)O)OC)C5═NC(C(NC(C)C)═O)═CS5 |
| Example 976 | [H]N2N═C(C1═CC═C(F)C═C1)C(═C2C3CCN(CC3)C(/C═C/C4═CC(O)═C(OC)C═C4)═O)C5═NC(C(NC(C)C)═O)═CS5 |
| Example 977 | [H]N2N═C(C1═CC═C(F)C═C1)C(═C2C3CCN(CC3)C(C4═CC═C(OCCCC)C═C4)═O)C5═NC(C(NC(C)C)═O)═CS5 |
| Example 978 | [H]N2N═C(C1═CC═C(F)C═C1)C(═C2C3CCN(CC3)C(C═5OC4═C(C═C(Cl)C═C4)C═5)═O)C6═NC(C(NC(C)C)═O)═CS6 |
| Example 979 | [H]N2N═C(C1═CC═C(F)C═C1)C(═C2C3CCN(CC3)C(C═4C═C(OC)C═CC═4[N+]([O−])═O)═O)C5═NC(C(NC(C)C)═O)═CS5 |
| Example 980 | [H]N2N═C(C1═CC═C(F)C═C1)C(═C2C3CCN(CC3)C(C4═CC(OC)═C(O)C(OC)═C4)═O)C5═NC(C(NC(C)C)═O)═CS5 |
| Example 981 | [H]N2N═C(C1═CC═C(F)C═C1)C(═C2C3CCN(CC3)C(═O)C═4C(═CC═CC═4)C5═CC═CC═C5)C6═NC(C(NC(C)C)═O)═CS6 |
| Example 982 | [H]N2N═C(C1═CC═C(F)C═C1)C(═C2C3CCN(CC3)C(C4═CC(═CC═C4)Br)═O)C5═NC(C(NC(C)C)═O)═CS5 |
| Example 983 | [H]N2N═C(C1═CC═C(F)C═C1)C(═C2C3CCN(CC3)C(C═4C═CC(Br)═CC═4)═O)C5═NC(C(NC(C)C)═O)═CS5 |
| Example 984 | [H]N2N═C(C1═CC═C(F)C═C1)C(═C2C3CCN(CC3)C(C4═CC═CC(═C4[N+](═O)[O−])Cl)═O)C5═NC(C(NC(C)C)═O)═CS5 |
| Example 985 | [H]N2N═C(C1═CC═C(F)C═C1)C(═C2C3CCN(CC3)C(C═4C(═CC═C([N+]([O−])═O)C═4)Cl)═O)C5═NC(C(NC(C)C)═O)═CS5 |
| Example 986 | [H]N2N═C(C1═CC═C(F)C═C1)C(═C2C3CCN(CC3)C(C4═C(C═C([N+]([O−])═O)C═C4)Cl)═O)C5═NC(C(NC(C)C)═O)═CS5 |
| Example 987 | [H]N2N═C(C1═CC═C(F)C═C1)C(═C2C3CCN(CC3)C(C4═CC═CC(C═C4[N+]([O−])═O)Cl)═O)C5═NC(C(NC(C)C)═O)═CS5 |
| Example 988 | [H]N2N═C(C1═CC═C(F)C═C1)C(═C2C3CCN(CC3)C(C═4C═C([N+]([O−])═O)C(═CC═4)Cl)═O)C5═NC(C(NC(C)C)═O)═CS5 |
| Example 989 | [H]N2N═C(C1═CC═C(F)C═C1)C(═C2C3CCN(CC3)C(C4═CC(Cl)═CC═C4 [N+](═O)[O−])═O)C5═NC(C(NC(C)C)═O)═CS5 |
| Example 990 | [H]N2N═C(C1═CC═C(F)C═C1)C(═C2C3CCN(CC3)C(═O)C═4N═C(Br)C═CC═4)C5═NC(C(NC(C)C)═O)═CS5 |
| Example 991 | [H]N2N═C(C1═CC═C(F)C═C1)C(═C2C3CCN(CC3)C(C═4C═NC═C(Br)C═4)═O)C5═NC(C(NC(C)C)═O)═CS5 |
| Example 992 | [H]N2N═C(C1═CC═C(F)C═C1)C(═C2C3CCN(CC3)C(C═4C([N+](═O)[O−])═CC(═C(C═4)F)F)═O)C5═NC(C(NC(C)C)═O)═CS5 |
| Example 993 | [H]N2N═C(C1═CC═C(F)C═C1)C(═C2C3CCN(CC3)C(CC═4C═CC(C(F)(F)F)═CC═4)═O)C5═NC(C(NC(C)C)═O)═CS5 |
| Example 994 | [H]N2N═C(C1═CC═C(F)C═C1)C(═C2C3CCN(CC3)C(C(C4═CC═C(CC(C)C)C═C4)C)═O)C5═NC(C(NC(C)C)═O)═CS5 |
| Example 995 | [H]N2N═C(C1═CC═C(F)C═C1)C(═C2C3CCN(CC3)C(C4═CC(═C(O)C(═C4)Cl)Cl)═O)C5═NC(C(NC(C)C)═O)═CS5 |
| Example 996 | [H]N2N═C(C1═CC═C(F)C═C1)C(═C2C3CCN(CC3)C(C═4C(═C(C(F)═C(C═4F)F)F)F)═O)C5═NC(C(NC(C)C)═O)═CS5 |
| Example 997 | [H]N2N═C(C1═CC═C(F)C═C1)C(═C2C3CCN(CC3)C(C4═C(OC)C═C(C═C4OC)OC)═O)C5═NC(C(NC(C)C)═O)═CS5 |
| Example 998 | [H]N2N═C(C1═CC═C(F)C═C1)C(═C2C3CCN(CC3)C(C4═C(C═C(C(═C4)OC)OC)OC)═O)C5═NC(C(NC(C)C)═O)═CS5 |
| Example 999 | [H]N2N═C(C1═CC═C(F)C═C1)C(═C2C3CCN(CC3)C(C4═CC(═C(OC)C(═C4)OC)OC)═O)C5═NC(C(NC(C)C)═O)═CS5 |
| Example 1000 | [H]N2N═C(C1═CC═C(F)C═C1)C(═C2C3CCN(CC3)C(C4═C(C(═C(OC)C═C4)OC)OC)═O)C5═NC(C(NC(C)C)═O)═CS5 |
| Example 1001 | [H]N2N═C(C1═CC═C(F)C═C1)C(═C2C3CCN(CC3)C(C5═C(CC═4C═CC═CC═4)C═CC═C5)═O)C6═NC(C(NC(C)C)═O)═CS6 |
| Example 1002 | [H]N2N═C(C1═CC═C(F)C═C1)C(═C2C3CCN(CC3)C(C5═CC═CC(OC4═CC═CC═C4)═C5)═O)C6═NC(C(NC(C)C)═O)═CS6 |
| Example 1003 | [H]N2N═C(C1═CC═C(F)C═C1)C(═C2C3CCN(CC3)C(C5═C(OC4═CC═CC═C4)C═CC═C5)═O)C6═NC(C(NC(C)C)═O)═CS6 |
| Example 1004 | [H]N2N═C(C1═CC═C(F)C═C1)C(═C2C3CCN(CC3)C(C5═CC═C(OC4═CC═CC═C4)C═C5)═O)C6═NC(C(NC(C)C)═O)═CS6 |
| Example 1005 | [H]N2N═C(C1═CC═C(F)C═C1)C(═C2C3CCN(CC3)C(C5═CC═C(C4═CC═C(O)C═C4)C═C5)═O)C6═NC(C(NC(C)C)═O)═CS6 |
| Example 1006 | [H]N2N═C(C1═CC═C(F)C═C1)C(═C2C3CCN(CC3)C(C4═CC(C)═C(C═C4)Br)═O)C5═NC(C(NC(C)C)═O)═CS5 |
| Example 1007 | [H]N2N═C(C1═CC═C(F)C═C1)C(═C2C3CCN(CC3)C(C4═CC(═C(C)C═C4)Br)═O)C5═NC(C(NC(C)C)═O)═CS5 |
| Example 1008 | [H]N2N═C(C1═CC═C(F)C═C1)C(═C2C3CCN(CC3)C(CC4═CC═C(Br)C═C4)═O)C5═NC(C(NC(C)C)═O)═CS5 |
| Example 1009 | [H]N2N═C(C1═CC═C(F)C═C1)C(═C2C3CCN(CC3)C(C4N(C(═O)OC(C)(C)C)CCC4)═O)C5═NC(C(NC(C)C)═O)═CS5 |
| Example 1010 | [H]N2N═C(C1═CC═C(F)C═C1)C(═C2C3CCN(CC3)C(/C═C/C4═C(C(F)(F)F)C═CC═C4)═O)C5═NC(C(NC(C)C)═O)═CS5 |
| Example 1011 | [H]N2N═C(C1═CC═C(F)C═C1)C(═C2C3CCN(CC3)C(C4═C(OCC)C═CC5═C4C═CC═C5)═O)C6═NC(C(NC(C)C)═O)═CS6 |
| Example 1012 | [H]N2N═C(C1═CC═C(F)C═C1)C(═C2C3CCN(CC3)C(C(NC(OC(C)(C)C)═O)C(C)C)═O)C4═NC(C(NC(C)C)═O)═CS4 |
| Example 1013 | [H]N2N═C(C1═CC═C(F)C═C1)C(═C2C3CCN(CC3)C(C(NC(OC(C)(C)C)═O)C(C)C)═O)C4═NC(C(NC(C)C)═O)═CS4 |
| Example 1014 | [H]N2N═C(C1═CC═C(F)C═C1)C(═C2C3CCN(CC3)C(CCC═4C═C(C═CC═4)C(F)(F)F)═O)C5═NC(C(NC(C)C)═O)═CS5 |
| Example 1015 | [H]N2N═C(C1═CC═C(F)C═C1)C(═C2C3CCN(CC3)C(C═5C═CC(N4N═C(C)CC4═O)═CC═5)═O)C6═NC(C(NC(C)C)═O)═CS6 |
| Example 1016 | [H]N2N═C(C1═CC═C(F)C═C1)C(═C2C3CCN(CC3)C(═O)C4═C(N═C(S4)Br)C)C5═NC(C(NC(C)C)═O)═CS5 |
| Example 1017 | [H]N2N═C(C1═CC═C(F)C═C1)C(═C2C3CCN(CC3)C(C═5C4═C(C═CC═C4)C═C6C═5C═CC═C6)═O)C7═NC(C(NC(C)C)═O)═CS7 |
| Example 1018 | [H]N2N═C(C1═CC═C(F)C═C1)C(═C2C3CCN(CC3)C(C═4C═C(C(C)C)C═C(C═4O)C(C)C)═O)C5═NC(C(NC(C)C)═O)═CS5 |
| Example 1019 | [H]N2N═C(C1═CC═C(F)C═C1)C(═C2C3CCN(CC3)C(C(NC(═O)C)CC4═CC═C(C═C4)O)═O)C5═NC(C(NC(C)C)═O)═CS5 |
| Example 1020 | [H]N2N═C(C1═CC═C(F)C═C1)C(═C2C3CCN(CC3)C(C═4C(═CC(Cl)═CC═4Cl)Cl)═O)C5═NC(C(NC(C)C)═O)═CS5 |
| Example 1021 | [H]N2N═C(C1═CC═C(F)C═C1)C(═C2C3CCN(CC3)C(C4═C(C═C(C(═C4)OC)OC)[N+](═O)[O−])═O)C5═NC(C(NC(C)C)═O)═CS5 |
| Example 1022 | [H]N2N═C(C1═CC═C(F)C═C1)C(═C2C3CCN(CC3)C(C5═CC═C(OCC4═CC═CC═C4)C═C5)═O)C6═NC(C(NC(C)C)═O)═CS6 |
| Example 1023 | [H]N2N═C(C1═CC═C(F)C═C1)C(═C2C3CCN(CC3)C(═O)CCC═4C═CC═CC═4Br)C5═NC(C(NC(C)C)═O)═CS5 |
| Example 1024 | [H]N2N═C(C1═CC═C(F)C═C1)C(═C2C3CCN(CC3)C(CCC═4C═C(Br)C═CC═4)═O)C5═NC(C(NC(C)C)═O)═CS5 |
| Example 1025 | [H]N2N═C(C1═CC═C(F)C═C1)C(═C2C3CCN(CC3)C(═O)C4N(CCCC4)C(═O)OC(C)(C)C)C5═NC(C(NC(C)C)═O)═CS5 |
| Example 1026 | [H]N2N═C(C1═CC═C(F)C═C1)C(═C2C3CCN(CC3)C(C4═CC═C(C═C4)OC5═CC(O)═CC═C5)═O)C6═NC(C(NC(C)C)═O)═CS6 |
| Example 1027 | [H]N2N═C(C1═CC═C(F)C═C1)C(═C2C3CCN(CC3)C(═O)C(C═5C═CC4═CC(═CC═C4C═5)OC)C)C6═NC(C(NC(C)C)═O)═CS6 |
| Example 1028 | [H]N2N═C(C1═CC═C(F)C═C1)C(═C2C3CCN(CC3)C(C4CN(C(OC(C)(C)C)═O)CCN4)═O)C5═NC(C(NC(C)C)═O)═CS5 |
| Example 1029 | [H]N2N═C(C1═CC═C(F)C═C1)C(═C2C3CCN(CC3)C(C4CN(C(OC(C)(C)C)═O)CCN4)═O)C5═NC(C(NC(C)C)═O)═CS5 |
| Example 1030 | [H]N2N═C(C1═CC═C(F)C═C1)C(═C2C3CCN(CC3)C(C4═C(C═CC(═C4)OC)Br)═O)C5═NC(C(NC(C)C)═O)═CS5 |
| Example 1031 | [H]N2N═C(C1═CC═C(F)C═C1)C(═C2C3CCN(CC3)C(C4CC(CN4C(═O)OC(C)(C)C)O)═O)C5═NC(C(NC(C)C)═O)═CS5 |
| Example 1032 | [H]N2N═C(C1═CC═C(F)C═C1)C(═C2C3CCN(CC3)C(C(C(C)C)N(C(OC(C)(C)C)═O)C)═O)C4═NC(C(NC(C)C)═O)═CS4 |
| Example 1033 | [H]N2N═C(C1═CC═C(F)C═C1)C(═C2C3CCN(CC3)C(C4═C(C═C(C(F)(F)F)C═C4)[N+]([O−])═O)═O)C5═NC(C(NC(C)C)═O)═CS5 |
| Example 1034 | [H]N2N═C(C1═CC═C(F)C═C1)C(═C2C3CCN(CC3)C(C4═CC(═CC═C4Cl)Br)═O)C5═NC(C(NC(C)C)═O)═CS5 |
| Example 1035 | [H]N2N═C(C1═CC═C(F)C═C1)C(═C2C3CCN(CC3)C(C═4C═C(Br)C(═CC═4)Cl)═O)C5═NC(C(NC(C)C)═O)═CS5 |
| Example 1036 | [H]N2N═C(C1═CC═C(F)C═C1)C(═C2C3CCN(CC3)C(C5═CC═CC═C5C(C═4C═CC(O)═CC═4)═O)═O)C6═NC(C(NC(C)C)═O)═CS6 |
| Example 1037 | [H]N2N═C(C1═CC═C(F)C═C1)C(═C2C3CCN(CC3)C(C5═C(C(C4═CC═C(F)C═C4)═O)C═CC═C5)═O)C6═NC(C(NC(C)C)═O)═CS6 |
| Example 1038 | [H]N2N═C(C1═CC═C(F)C═C1)C(═C2C3CCN(CC3)C(C(C5═CC(═C(C4═CC═CC═C4)C═C5)F)C)═O)C6═NC(C(NC(C)C)═O)═CS6 |
| Example 1039 | [H]N2N═C(C1═CC═C(F)C═C1)C(═C2C3CCN(CC3)C(C═4C═CC(l)═CC═4)═O)C5═NC(C(NC(C)C)═O)═CS5 |
| Example 1040 | [H]N2N═C(C1═CC═C(F)C═C1)C(═C2C3CCN(CC3)C(C4═CC(═CC═C4)l)═O)C5═NC(C(NC(C)C)═O)═CS5 |
| Example 1041 | [H]N2N═C(C1═CC═C(F)C═C1)C(═C2C3CCN(CC3)C(C5N(C(OCC4═CC═CC═C4)═O)CCC5)═O)C6═NC(C(NC(C)C)═O)═CS6 |
| Example 1042 | [H]N2N═C(C1═CC═C(F)C═C1)C(═C2C3CCN(CC3)C(C(C(CC)C)NC(OC(C)(C)C)═O)═O)C4═NC(C(NC(C)C)═O)═CS4 |
| Example 1043 | [H]N2N═C(C1═CC═C(F)C═C1)C(═C2C3CCN(CC3)C(C(NC(OC(C)(C)C)═O)CC(C)C)═O)C4═NC(C(NC(C)C)═O)═CS4 |
| Example 1044 | [H]N2N═C(C1═CC═C(F)C═C1)C(═C2C3CCN(CC3)C(C(C5═CC(C(C4═CC═CC═C4)═O)═CC═C5)C)═O)C6═NC(C(NC(C)C)═O)═CS6 |
| Example 1045 | [H]N2N═C(C1═CC═C(F)C═C1)C(═C2C3CCN(CC3)C(C(NC(OC(C)(C)C)═O)CC4═CC═CC═C4)═O)C5═NC(C(NC(C)C)═O)═CS5 |
| Example 1046 | [H]N2N═C(C1═CC═C(F)C═C1)C(═C2C3CCN(CC3)C(C(NC(OC(C)(C)C)═O)CC4═NC═CC═C4)═O)C5═NC(C(NC(C)C)═O)═CS5 |
| Example 1047 | [H]N2N═C(C1═CC═C(F)C═C1)C(═C2C3CCN(CC3)C(C4CC═5C═CC═CC═5CN4C(═O)OC(C)(C)C)═O)C6═NC(C(NC(C)C)═O)═CS6 |
| Example 1048 | [H]N2N═C(C1═CC═C(F)C═C1)C(═C2C3CCN(CC3)C(C(CC4═CC═C(O)C═C4)NC(OC(C)(C)C)═O)═O)C5═NC(C(NC(C)C)═O)═CS5 |

### EXAMPLES 1049-1374

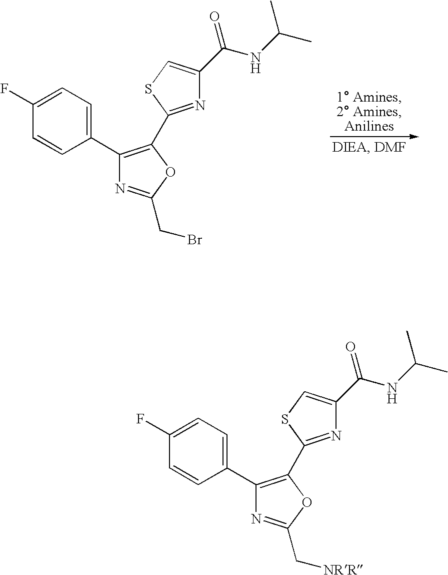 Starting bromomethyl oxazole was prepared as described in Step 1 of Example 44. Where, 1° amines, 2° amines, and anilines were selected to afford Examples 1049-1374, which were prepared by General Procedure 2.

Examples Prepared with 1° Amines:

| | |
|---|---|
| Example 1049 | N1═C(SC═C1C(═O)NC(C)C)C═2OC(CNC)═NC═2C3═CC═C(F)C═C3 |
| Example 1050 | N1═C(SC═C1C(═O)NC(C)C)C═2OC(CNCC)═NC═2C3═CC═C(F)C═C3 |
| Example 1051 | N1═C(SC═C1C(═O)NC(C)C)C═2OC(CNCC#C)═NC═2C3═CC═C(F)C═C3 |
| Example 1052 | N1═C(SC═C1C(═O)NC(C)C)C═2OC(CNCC#N)═NC═2C3═CC═C(F)C═C3 |
| Example 1053 | N1═C(SC═C1C(═O)NC(C)C)C═3OC(CNC2CC2)═NC═3C4═CC═C(F)C═C4 |
| Example 1054 | N1═C(SC═C1C(═O)NC(C)C)C═2OC(CNC(C)C)═NC═2C3═CC═C(F)C═C3 |
| Example 1055 | N1═C(SC═C1C(═O)NC(C)C)C═2OC(CNCCC)═NC═2C3═CC═C(F)C═C3 |
| Example 1056 | N1═C(SC═C1C(═O)NC(C)C)C═2OC(CNCCN)═NC═2C3═CC═C(F)C═C3 |
| Example 1057 | N1═C(SC═C1C(═O)NC(C)C)C═2OC(CNCCO)═NC═2C3═CC═C(F)C═C3 |
| Example 1058 | N1═C(SC═C1C(═O)NC(C)C)C═2OC(CNCCC#N)═NC═2C3═CC═C(F)C═C3 |
| Example 1059 | N1═C(SC═C1C(═O)NC(C)C)C═3OC(CNC2CCC2)═NC═3C4═CC═C(F)C═C4 |
| Example 1060 | N1═C(SC═C1C(═O)NC(C)C)C═3OC(CNCC2CC2)═NC═3C4═CC═C(F)C═C4 |
| Example 1061 | N1═C(SC═C1C(═O)NC(C)C)C═2OC(CNC(CC)C)═NC═2C3═CC═C(F)C═C3 |
| Example 1062 | N1═C(SC═C1C(═O)NC(C)C)C═2OC(CNCC(C)C)═NC═2C3═CC═C(F)C═C3 |
| Example 1063 | N1═C(SC═C1C(═O)NC(C)C)C═2OC(CNCCCO)═NC═2C3═CC═C(F)C═C3 |
| Example 1064 | N1═C(SC═C1C(═O)NC(C)C)C═2OC(CNC(C)CO)═NC═2C3═CC═C(F)C═C3 |
| Example 1065 | N1═C(SC═C1C(═O)NC(C)C)C═2OC(CNCCOC)═NC═2C3═CC═C(F)C═C3 |
| Example 1066 | N1═C(SC═C1C(═O)NC(C)C)C═3OC(CNC2CCCC2)═NC═3C4═CC═C(F)C═C4 |
| Example 1067 | N1═C(SC═C1C(═O)NC(C)C)C═2OC(CNC(CCC)C)═NC═2C3═CC═C(F)C═C3 |
| Example 1068 | N1═C(SC═C1C(═O)NC(C)C)C═2OC(CNCC(CC)C)═NC═2C3═CC═C(F)C═C3 |
| Example 1069 | N1═C(SC═C1C(═O)NC(C)C)C═2OC(CNC(C(C)C)C)═NC═2C3═CC═C(F)C═C3 |
| Example 1070 | N1═C(SC═C1C(═O)NC(C)C)C═2OC(CNCCC(C)C)═NC═2C3═CC═C(F)C═C3 |
| Example 1071 | N1═C(SC═C1C(═O)NC(C)C)C═2OC(CNCCN(C)C)═NC═2C3═CC═C(F)C═C3 |
| Example 1072 | N1═C(SC═C1C(═O)NC(C)C)C═2OC(CNC(COC)C)═NC═2C3═CC═C(F)C═C3 |
| Example 1073 | N1═C(SC═C1C(═O)NC(C)C)C═2OC(CNC(CC)CO)═NC═2C3═CC═C(F)C═C3 |
| Example 1074 | N1═C(SC═C1C(═O)NC(C)C)C═2OC(CNC(CC)CO)═NC═2C3═CC═C(F)C═C3 |
| Example 1075 | N1═C(SC═C1C(═O)NC(C)C)C═2OC(CNCCCCO)═NC═2C3═CC═C(F)C═C3 |
| Example 1076 | N1═C(SC═C1C(═O)NC(C)C)C═3OC(CNCC═2OC═CC═2)═NC═3C4═CC═C(F)C═C4 |
| Example 1077 | N1═C(SC═C1C(═O)NC(C)C)C═3OC(CNC2CCCCC2)═NC═3C4═CC═C(F)C═C4 |
| Example 1078 | N1═C(SC═C1C(═O)NC(C)C)C═3OC(CNCC2CCCO2)═NC═3C4═CC═C(F)C═C4 |
| Example 1079 | N1═C(SC═C1C(═O)NC(C)C)C═2OC(CNCCC(C)(C)C)═NC═2C3═CC═C(F)C═C3 |
| Example 1080 | N1═C(SC═C1C(═O)NC(C)C)C═2OC(CNC(CC(C)C)C)═NC═2C3═CC═C(F)C═C3 |
| Example 1081 | N1═C(SC═C1C(═O)NC(C)C)C═3OC(CNC2CONC2═O)═NC═3C4═CC═C(F)C═C4 |
| Example 1082 | N1═C(SC═C1C(═O)NC(C)C)C═3OC(CNN2CCOCC2)═NC═3C4═CC═C(F)C═C4 |
| Example 1083 | N1═C(SC═C1C(═O)NC(C)C)C═2OC(CNC(C(C)C)CO)═NC═2C3═CC═C(F)C═C3 |
| Example 1084 | N1═C(SC═C1C(═O)NC(C)C)C═3OC(CNCC2═CC═CC═C2)═NC═3C4═CC═C(F)C═C4 |
| Example 1085 | N1═C(SC═C1C(═O)NC(C)C)C═3OC(CNCC2═CC═CN═C2)═NC═3C4═CC═C(F)C═C4 |
| Example 1086 | N1═C(SC═C1C(═O)NC(C)C)C═3OC(CNCC2═CC═CC═N2)═NC═3C4═CC═C(F)C═C4 |
| Example 1087 | N1═C(SC═C1C(═O)NC(C)C)C═3OC(CNCC2═CC═NC═C2)═NC═3C4═CC═C(F)C═C4 |
| Example 1088 | N1═C(SC═C1C(═O)NC(C)C)C═3OC(CNCC2═CC═CS2)═NC═3C4═CC═C(F)C═C4 |
| Example 1089 | N1═C(SC═C1C(═O)NC(C)C)C═3OC(CNC2CCC(C)CC2)═NC═3C4═CC═C(F)C═C4 |
| Example 1090 | N1═C(SC═C1C(═O)NC(C)C)C═3OC(CNCC2CCCCC2)═NC═3C4═CC═C(F)C═C4 |
| Example 1091 | N1═C(SC═C1C(═O)NC(C)C)C═3OC(CNC2CCCCCC2)═NC═3C4═CC═C(F)C═C4 |
| Example 1092 | N1═C(SC═C1C(═O)NC(C)C)C═3OC(CNC2CCCCC2C)═NC═3C4═CC═C(F)C═C4 |
| Example 1093 | N1═C(SC═C1C(═O)NC(C)C)C═3OC(CNCCN2CCCC2)═NC═3C4═CC═C(F)C═C4 |
| Example 1094 | N1═C(SC═C1C(═O)NC(C)C)C═3OC(CNN2CCN(C)CC2)═NC═3C4═CC═C(F)C═C4 |
| Example 1095 | N1═C(SC═C1C(═O)NC(C)C)C═2OC(CNCCN(CC)CC)═NC═2C3═CC═C(F)C═C3 |
| Example 1096 | N1═C(SC═C1C(═O)NC(C)C)C═3OC(CNCC═2C═CC═C(C)C═2)═NC═3C4═CC═C(F)C═C4 |
| Example 1097 | N1═C(SC═C1C(═O)NC(C)C)C═3OC(CN[C@@H](C)C2═CC═CC═C2)═NC═3C4═CC═C(F)C═C4 |
| Example 1098 | N1═C(SC═C1C(═O)NC(C)C)C═3OC(CN[C@H](C)C2═CC═CC═C2)═NC═3C4═CC═C(F)C═C4 |
| Example 1099 | N1═C(SC═C1C(═O)NC(C)C)C═3OC(CNCCC2═CC═CC═C2)═NC═3C4═CC═C(F)C═C4 |
| Example 1100 | N1═C(SC═C1C(═O)NC(C)C)C═3OC(CNCC2═CC═CC═C2C)═NC═3C4═CC═C(F)C═C4 |
| Example 1101 | N1═C(SC═C1C(═O)NC(C)C)C═3OC(CNCC2═CC═C(C)C═C2)═NC═3C4═CC═C(F)C═C4 |
| Example 1102 | N1═C(SC═C1C(═O)NC(C)C)C═3OC(CNCC2═NC═C(C)N═C2)═NC═3C4═CC═C(F)C═C4 |
| Example 1103 | N1═C(SC═C1C(═O)NC(C)C)C═3OC(CNCC2═CC═CC(F)═C2)═NC═3C4═CC═C(F)C═C4 |
| Example 1104 | N1═C(SC═C1C(═O)NC(C)C)C═3OC(CNCC2═CC═C(F)C═C2)═NC═3C4═CC═C(F)C═C4 |
| Example 1105 | N1═C(SC═C1C(═O)NC(C)C)C═3OC(CNCC2═CC═CC═C2F)═NC═3C4═CC═C(F)C═C4 |
| Example 1106 | N1═C(SC═C1C(═O)NC(C)C)C═3OC(CNCCCN2C═CN═C2)═NC═3C4═CC═C(F)C═C4 |
| Example 1107 | N1═C(SC═C1C(═O)NC(C)C)C═3OC(CNCCC2CCCN2C)═NC═3C4═CC═C(F)C═C4 |
| Example 1108 | N1═C(SC═C1C(═O)NC(C)C)C═3OC(CNCCN2CCCCC2)═NC═3C4═CC═C(F)C═C4 |
| Example 1109 | N1═C(SC═C1C(═O)NC(C)C)C═2OC(CNC(CCCC(C)C)C)═NC═2C3═CC═C(F)C═C3 |
| Example 1110 | N1═C(SC═C1C(═O)NC(C)C)C═3OC(CNCCN2CCOCC2)═NC═3C4═CC═C(F)C═C4 |
| Example 1111 | N1═C(SC═C1C(═O)NC(C)C)C═2OC(CNC(CC(OCC)═O)C)═NC═2C3═CC═C(F)C═C3 |
| Example 1112 | N1═C(SC═C1C(═O)NC(C)C)C═4OC(CNC2CCC3═CC═CC═C23)═NC═4C5═CC═C(F)C═C5 |
| Example 1113 | N1═C(SC═C1C(═O)NC(C)C)C═3OC(CNCCC2CC═C(C)C═C2)═NC═3C4═CC═C(F)C═C4 |
| Example 1114 | N1═C(SC═C1C(═O)NC(C)C)C═3OC(CNCC═2C(═CC═C(C)C═2)C)═NC═3C4═CC═C(F)C═C4 |
| Example 1115 | N1═C(SC═C1C(═O)NC(C)C)C═3OC(CN[C@@H](C)C2═CC═C(C)C═C2)═NC═3C4═CC═C(F)C═C4 |
| Example 1116 | N1═C(SC═C1C(═O)NC(C)C)C═3OC(CN[C@H](C)C2═CC═C(C)C═C2)═NC═3C4═CC═C(F)C═C4 |
| Example 1117 | N1═C(SC═C1C(═O)NC(C)C)C═3OC(CNCC(C)C2═CC═CC═C2)═NC═3C4═CC═C(F)C═C4 |
| Example 1118 | N1═C(SC═C1C(═O)NC(C)C)C═3OC(CNCC(C)C2═CC═CC═C2)═NC═3C4═CC═C(F)C═C4 |
| Example 1119 | N1═C(SC═C1C(═O)NC(C)C)C═3OC(CNCCCC2═CC═CC═C2)═NC═3C4═CC═C(F)C═C4 |
| Example 1120 | N1═C(SC═C1C(═O)NC(C)C)C═3OC(CNCC2═CC═C(C)C(═C2)C)═NC═3C4═CC═C(F)C═C4 |
| Example 1121 | N1═C(SC═C1C(═O)NC(C)C)C═3OC(CNC(C)CC2═CC═NC═C2)═NC═3C4═CC═C(F)C═C4 |
| Example 1122 | N1═C(SC═C1C(═O)NC(C)C)C═3OC(CNCCC2═CC═C(O)C═C2)═NC═3C4═CC═C(F)C═C4 |
| Example 1123 | N1═C(SC═C1C(═O)NC(C)C)C═3OC(CNCCOC2═CC═CC═C2)═NC═3C4═CC═C(F)C═C4 |
| Example 1124 | N1═C(SC═C1C(═O)NC(C)C)C═3OC(CNCC2═CC═CC═C2OC)═NC═3C4═CC═C(F)C═C4 |
| Example 1125 | N1═C(SC═C1C(═O)NC(C)C)C═3OC(CNCC2═CC═C(OC)C═C2)═NC═3C4═CC═C(F)C═C4 |
| Example 1126 | N1═C(SC═C1C(═O)NC(C)C)C═3OC(CNCC═2C═CC═C(OC)C═2)═NC═3C4═CC═C(F)C═C4 |
| Example 1127 | N1═C(SC═C1C(═O)NC(C)C)C═3OC(CNCCC2═CC═C(F)C═C2)═NC═3C4═CC═C(F)C═C4 |
| Example 1128 | N1═C(SC═C1C(═O)NC(C)C)C═3OC(CNCCC2═CC═CC═C2F)═NC═3C4═CC═C(F)C═C4 |
| Example 1129 | N1═C(SC═C1C(═O)NC(C)C)C═3OC(CNCCC2═CC═CC(F)═C2)═NC═3C4═CC═(F)C═C4 |
| Example 1130 | N1═C(SC═C1C(═O)NC(C)C)C═3OC(CNCC2═CC═CC(Cl)═C2)═NC═3C4═CC═C(F)C═C4 |
| Example 1131 | N1═C(SC═C1C(═O)NC(C)C)C═3OC(CNCC2═CC═CC═C2Cl)═NC═3C4═CC═C(F)C═C4 |
| Example 1132 | N1═C(SC═C1C(═O)NC(C)C)C═3OC(CNCC2═CC═C(Cl)C═C2)═NC═3C4═CC═C(F)C═C4 |
| Example 1133 | N1═C(SC═C1C(═O)NC(C)C)C═3OC(CNCCCN2CCCC2═O)═NC═3C4═CC═C(F)C═C4 |
| Example 1134 | N1═C(SC═C1C(═O)NC(C)C)C═3OC(CNCC2═CC═C(F)C═C2F)═NC═3C4═CC═C(F)C═C4 |
| Example 1135 | N1═C(SC═C1C(═O)NC(C)C)C═3OC(CNCC2═CC═C(F)C(F)═C2)═NC═3C4═CC═C(F)C═C4 |
| Example 1136 | N1═C(SC═C1C(═O)NC(C)C)C═3OC(CNCC2═CC(F)═CC(F)═C2)═NC═3C4═CC═C(F)C═C4 |
| Example 1137 | N1═C(SC═C1C(═O)NC(C)C)C═3OC(CNCCCN2CCOCC2)═NC═3C4═CC═C(F)C═C4 |
| Example 1138 | N1═C(SC═C1C(═O)NC(C)C)C═2OC(CNCCN(C(C)C)C(C)C)═NC═2C3═CC═C(F)C═C3 |
| Example 1139 | N1═C(SC═C1C(═O)NC(C)C)C═4OC(CNC2CCCC3═CC═CC═C23)═NC═4C5═CC═C(F)C═C5 |
| Example 1140 | N1═C(SC═C1C(═O)NC(C)C)C═3OC(CNC(C)CCC2═CC═CC═C2)═NC═3C4═CC═C(F)C═C4 |
| Example 1141 | N1═C(SC═C1C(═O)NC(C)C)C═3OC(CNCC2═CC═C(C(C)C)C═C2)═NC═3C4═CC═C(F)C═C4 |
| Example 1142 | N1═C(SC═C1C(═O)NC(C)C)C═4OC(CNCC3═CC═C2OCOC2═C3)═NC═4C5═CC═C(F)C═C5 |
| Example 1143 | N1═C(SC═C1C(═O)NC(C)C)C═3OC(CNCCC═2C═CC═C(OC)C═2)═NC═3C4═CC═C(F)C═C4 |
| Example 1144 | N1═C(SC═C1C(═O)NC(C)C)C═3OC(CN[C@H](CO)CC2═CC═CC═C2)═NC═3C4═CC═C(F)C═C4 |
| Example 1145 | N1═C(SC═C1C(═O)NC(C)C)C═3OC(CNCCC2═CC═C(OC)C═C2)═NC═3C4═CC═C(F)C═C4 |
| Example 1146 | N1═C(SC═C1C(═O)NC(C)C)C═3OC(CNCCC2═CC═CC═C2OC)═NC═3C4═CC═C(F)C═C4 |
| Example 1147 | N1═C(SC═C1C(═O)NC(C)C)C═3OC(CNCC2═CC═CC═C2OCC)═NC═3C4═CC═C(F)C═C4 |
| Example 1148 | N1═C(SC═C1C(═O)NC(C)C)C═3OC(CNC2═CC═C(OC)C(═C2)OC)═NC═3C4═CC═C(F)C═C4 |
| Example 1149 | N1═C(SC═C1C(═O)NC(C)C)C═3OC(CNCCC2═CC═CC═C2Cl)═NC═3C4═CC═C(F)C═C4 |
| Example 1150 | N1═C(SC═C1C(═O)NC(C)C)C═3OC(CNCCC2═CC═C(Cl)C═C2)═NC═3C4═CC═C(F)C═C4 |
| Example 1151 | N1═C(SC═C1C(═O)NC(C)C)C═3OC(CNCCC2═CC═CC(Cl)═C2)═NC═3C4═CC═C(F)C═C4 |
| Example 1152 | N1═C(SC═C1C(═O)NC(C)C)C═3OC(CNC2CC(C)(C)NC(C2)(C)C)═NC═3C4═CC═C(F)C═C4 |
| Example 1153 | N1═C(SC═C1C(═O)NC(C)C)C═2OC(CNC(CCCN(CC)CC)C)═NC═2C3═CC═C(F)C═C3 |
| Example 1154 | N1═C(SC═C1C(═O)NC(C)C)C═3OC(CNCC2═CC═C(F)C(Cl)═C2)═NC═3C4═CC═C(F)C═C4 |
| Example 1155 | N1═C(SC═C1C(═O)NC(C)C)C═3OC(CN(CCC#N)CC2═CC═CC═C2)═NC═3C4═CC═C(F)C═C4 |
| Example 1156 | N1═C(SC═C1C(═O)NC(C)C)C═2OC(CNCCNC(OC(C)(C)C)═O)═NC═2C3═CC═C(F)C═C3 |
| Example 1157 | [H]N4C═C(CCNCC═2OC(C═1SC═C(C(═O)NC(C)C)N═1)═C(N═2)C3═CC═C(F)C═C3)C5═CC═CC═C45 |
| Example 1158 | N1═C(SC═C1C(═O)NC(C)C)C═3OC(CNCC2═CC═C(C(C)(C)C)C═C2)═NC═3C4═CC═C(F)C═C4 |
| Example 1159 | N1═C(SC═C1C(═O)NC(C)C)C═3OC(CNCCCN(C)C2═CC═CC═C2)═NC═3C4═CC═C(F)C═C4 |
| Example 1160 | N1═C(SC═C1C(═O)NC(C)C)C═3OC(CNCC2(O)CCCCC2)═NC═3C4═CC═C(F)C═C4 |
| Example 1161 | N1═C(SC═C1C(═O)NC(C)C)C═3OC(CNCC═2C═C(OC)C═C(C═2)OC)═NC═3C4═CC═C(F)C═C4 |
| Example 1162 | N1═C(SC═C1C(═O)NC(C)C)C═3OC(CNCC2═CC═C(OC)C(═C2)OC)═NC═3C4═CC═C(F)C═C4 |
| Example 1163 | N1═C(SC═C1C(═O)NC(C)C)C═3OC(CNCC(═O)C2═CC═CC═C2)═NC═3C4═CC═C(F)C═C4 |
| Example 1164 | N1═C(SC═C1C(═O)NC(C)C)C═3OC(CNC2CCN(C(OCC)═O)CC2)═NC═3C4═CC═C(F)C═C4 |
| Example 1165 | N1═C(SC═C1C(═O)NC(C)C)C═2OC(CNCCCNC(OC(C)(C)C)═O)═NC═2C3═CC═C(F)C═C3 |
| Example 1166 | N1═C(SC═C1C(═O)NC(C)C)C═3OC(CNCC2═CC═C(C═C2)C(F)(F)F)═NC═3C4═CC═C(F)C═C4 |
| Example 1167 | N1═C(SC═C1C(═O)NC(C)C)C═3OC(CNCC2═CC═CC(═C2)C(F)(F)F)═NC═3C4═CC═C(F)C═C4 |
| Example 1168 | N1═C(SC═C1C(═O)NC(C)C)C═3OC(CNCC2═CC═C(Cl)C(Cl)═C2)═NC═3C4═CC═C(F)C═C4 |
| Example 1169 | N1═C(SC═C1C(═O)NC(C)C)C═3OC(CNCC2═CC═C(Cl)C═C2Cl)═NC═3C4═CC═C(F)C═C4 |
| Example 1170 | N1═C(SC═C1C(═O)NC(C)C)C═4OC(CNC3CCN(CC2═CC═CC═C2)C3)═NC═4C5═CC═C(F)C═C5 |
| Example 1171 | N1═C(SC═C1C(═O)NC(C)C)C═3OC(CNCCC═2C(═CC═C(OC)C═2)OC)═NC═3C4═CC═C(F)C═C4 |
| Example 1172 | N1═C(SC═C1C(═O)NC(C)C)C═3OC(CNCC2═CC═C(N(C)C)C═C2)═NC═3C4═CC═C(F)C═C4 |
| Example 1173 | N1═C(SC═C1C(═O)NC(C)C)C═3OC(CNCC2═CC═C(OC)C(═C2)O)═NC═3C4═CC═C(F)C═C4 |
| Example 1174 | N1═C(SC═C1C(═O)NC(C)C)C═4OC(CNC3CCN(CC2═CC═CC═C2)CC3)═NC═4C5═CC═C(F)C═C5 |
| Example 1175 | N1═C(SC═C1C(═O)NC(C)C)C═3OC(CNCC2═CC═CC═C2OC(F)(F)F)═NC═3C4═CC═C(F)C═C4 |
| Example 1176 | N1═C(SC═C1C(═O)NC(C)C)C═3OC(CNCCC2═CC═C(C═C2)S(═O)(═O)N)═NC═3C4═CC═C(F)C═C4 |
| Example 1177 | N1═C(SC═C1C(═O)NC(C)C)C═3OC(CNCC(C2═CC═C(OC)C═C2)═O)═NC═3C4═CC═C(F)C═C4 |
| Example 1178 | N1═C(SC═C1C(═O)NC(C)C)C═4OC(CNCCC3═CC═C2OCOC2═C3)═NC═4C5═CC═C(F)C═C5 |
| Example 1179 | N1═C(SC═C1C(═O)NC(C)C)C═4OC(CNCCC(C2═CC═CC═C2)C3═CC═CC═C3)═NC═4C5═CC═C(F)C═C5 |
| Example 1180 | N1═C(SC═C1C(═O)NC(C)C)C═3OC(CNCCNS(C2═CC═C(C)C═C2)(═O)═O)═NC═3C4═CC═C(F)C═C4 |
| Example 1181 | N1═C(SC═C1C(═O)NC(C)C)C═2OC(CBr)═NC═2C3═CC═C(F)C═C3 |

Examples Prepared with 20 Amines:

| | |
|---|---|
| Example 1182 | CN(CC1═NC(═C(O1)C═2SC═C(N═2)C(═O)N(C(C)C)[H])C3═CC═C(C═C3)F)C |
| Example 1183 | C4N(CC1═NC(═C(O1)C═2SC═C(N═2)C(═O)N(C(C)C)[H])C3═CC═C(C═C3)F)CCOC4 |
| Example 1184 | N1═C(SC═C1C(═O)NC(C)C)C═3OC(CN(CCC#N)CC2═CC═CC═C2)═NC═3C4═CC═C(F)C═C4 |
| Example 1185 | N1═C(SC═C1C(═O)NC(C)C)C═2OC(CBr)═NC═2C3═CC═C(F)C═C3 |
| Example 1186 | N1═C(SC═C1C(═O)NC(C)C)C═2OC(CN(CC)C)═NC═2C3═CC═C(F)C═C3 |
| Example 1187 | N1═C(SC═C1C(═O)NC(C)C)C═2OC(CN(C)CC#C)═NC═2C3═CC═C(F)C═C3 |
| Example 1188 | N1═C(SC═C1C(═O)NC(C)C)C═2OC(CN(CC)CC)═NC═2C3═CC═C(F)C═C3 |
| Example 1189 | N1═C(SC═C1C(═O)NC(C)C)C═2OC(CN(CCC)C)═NC═2C3═CC═C(F)C═C3 |
| Example 1190 | N1═C(SC═C1C(═O)NC(C)C)C═2OC(CN(C)CCO)═NC═2C3═CC═C(F)C═C3 |
| Example 1191 | N1═C(SC═C1C(═O)NC(C)C)C═2OC(CN(C)CCC #N)═NC═2C3═CC═C(F)C═C3 |
| Example 1192 | N1═C(SC═C1C(═O)NC(C)C)C═2OC(CN(C)CC(C)C)═NC═2C3═CC═C(F)C═C3 |
| Example 1193 | N1═C(SC═C1C(═O)NC(C)C)C═2OC(CN(CC)C(C)C)═NC═2C3═CC═C(F)C═C3 |
| Example 1194 | N1═C(SC═C1C(═O)NC(C)C)C═2OC(CN(CCCC)C)═NC═2C3═CC═C(F)C═C3 |
| Example 1195 | N1═C(SC═C1C(═O)NC(C)C)C═2OC(CN(CC)CCO)═NC═2C3═CC═C(F)C═C3 |
| Example 1196 | N1═C(SC═C1C(═O)NC(C)C)C═3OC(CN2CSCC2)═NC═3C4═CC═C(F)C═C4 |
| Example 1197 | N1═C(SC═C1C(═O)NC(C)C)C═3OC(CN2CCCCC2C)═NC═3C4═CC═C(F)C═C4 |
| Example 1198 | N1═C(SC═C1C(═O)NC(C)C)C═3OC(CN2CCC(C)CC2)═NC═3C4═CC═C(F)C═C4 |
| Example 1199 | N1═C(SC═C1C(═O)NC(C)C)C═3OC(CN2CCN(C)CC2)═NC═3C4═CC═C(F)C═C4 |
| Example 1200 | N1═C(SC═C1C(═O)NC(C)C)C═3OC(CN2CCCC2CO)═NC═3C4═CC═C(F)C═C4 |
| Example 1201 | N1═C(SC═C1C(═O)NC(C)C)C═3OC(CN2CCC[C@@H]2CO)═NC═3C4═CC═C(F)C═C4 |
| Example 1202 | N1═C(SC═C1C(═O)NC(C)C)C═2OC(CN(CCCC)CC)═NC═2C3═CC═C(F)C═C3 |
| Example 1203 | N1═C(SC═C1C(═O)NC(C)C)C═2OC(CN(CCC)CCC)═NC═2C3═CC═C(F)C═C3 |
| Example 1204 | N1═C(SC═C1C(═O)NC(C)C)C═3OC(CN2CCSCC2)═NC═3C4═CC═C(F)C═C4 |
| Example 1205 | N1═C(SC═C1C(═O)NC(C)C)C═2OC(CN(CCO)CCO)═NC═2C3═CC═C(F)C═C3 |
| Example 1206 | N1═C(SC═C1C(═O)NC(C)C)C═3OC(CN2CC(C)CC(C2)C)═NC═3C4═CC═C(F)C═C4 |
| Example 1207 | N1═C(SC═C1C(═O)NC(C)C)C═3OC(CN(C)C2CCCCC2)═NC═3C4═CC═C(F)C═C4 |
| Example 1208 | N1═C(SC═C1C(═O)NC(C)C)C═3OC(CN2C(CCCC2C)C)═NC═3C4═CC═C(F)C═C4 |
| Example 1209 | N1═C(SC═C1C(═O)NC(C)C)C═3OC(CN2CCC(N(C)C)C2)═NC═3C4═CC═C(F)C═C4 |
| Example 1210 | N1═C(SC═C1C(═O)NC(C)C)C═3OC(CN2C[C@@H](C)N[C@H](C2)C)═NC═3C4═CC═C(F)C═C4 |
| Example 1211 | N1═C(SC═C1C(═O)NC(C)C)C═3OC(CN2CCCC(CO)C2)═NC═3C4═CC═C(F)C═C4 |
| Example 1212 | N1═C(SC═C1C(═O)NC(C)C)C═3OC(CN2CCC[C@H]2COC)═NC═3C4═CC═C(F)C═C4 |
| Example 1213 | N1═C(SC═C1C(═O)NC(C)C)C═3OC(CN2CC(C)OC(C2)C)═NC═3C4═CC═(F)C═C4 |
| Example 1214 | N1═C(SC═C1C(═O)NC(C)C)C═3OC(CN2CCCCC2CO)═NC═3C4═CC═C(F)C═C4 |
| Example 1215 | N1═C(SC═C1C(═O)NC(C)C)C═2OC(CN(CC)CCCCO)═NC═2C3═CC═C(F)C═C3 |
| Example 1216 | N1═C(SC═C1C(═O)NC(C)C)C═4OC(CN2CC3═CC═CC═C3C2)═NC═4C5═CC═C(F)C═C5 |
| Example 1217 | N1═C(SC═C1C(═O)NC(C)C)C═3OC(CN(C)CC2═CC═CC═C2)═NC═3C4═CC═C(F)C═C4 |
| Example 1218 | N1═C(SC═C1C(═O)NC(C)C)C═3OC(CN(CC)C2CCCCC2)═NC═3C4═CC═C(F)C═C4 |
| Example 1219 | N1═C(SC═C1C(═O)NC(C)C)C═3OC(CN2CCC(C(N)═O)CC2)═NC═3C4═CC═C(F)C═C4 |
| Example 1220 | N1═C(SC═C1C(═O)NC(C)C)C═3OC(CN2CC[C@H](NC(C)═O)C2)═NC═3C4═CC═C(F)C═C4 |
| Example 1221 | N1═C(SC═C1C(═O)NC(C)C)C═3OC(CN2CC[C@H](NC(C)═O)C2)═NC═3C4═CC═C(F)C═C4 |
| Example 1222 | N1═C(SC═C1C(═O)NC(C)C)C═3OC(CN2CCCC(C(N)═O)C2)═NC═3C4═CC═C(F)C═C4 |
| Example 1223 | N1═C(SC═C1C(═O)NC(C)C)C═3OC(CN2CCCCC2CCO)═NC═3C4═CC═C(F)C═C4 |
| Example 1224 | N1═C(SC═C1C(═O)NC(C)C)C═4OC(CN2CC3═CC═CC═C3CC2)═NC═4C5═CC═C(F)C═C5 |
| Example 1225 | N1═C(SC═C1C(═O)NC(C)C)C═3OC(CN(CC)CC2═CC═CC═C2)═NC═3C4═CC═C(F)C═C4 |
| Example 1226 | N1═C(SC═C1C(═O)NC(C)C)C═3OC(CN(CC)CC2═CC═CC═C2)═NC═3C4═CC═C(F)C═C4 |
| Example 1227 | N1═C(SC═C1C(═O)NC(C)C)C═3OC(CN(C)CCC2═CC═CC═C2)═NC═3C4═CC═C(F)C═C4 |
| Example 1228 | N1═C(SC═C1C(═O)NC(C)C)C═3OC(CN(C)CCC2═CC═CC═N2)═NC═3C4═CC═C(F)C═C4 |
| Example 1229 | N1═C(SC═C1C(═O)NC(C)C)C═3OC(CN(C)CCC2═CC═CC═N2)═NC═3C4═CC═C(F)C═C4 |
| Example 1230 | N1═C(SC═C1C(═O)NC(C)C)C═4OC(CN2C3CCCCC3CCC2)═NC═4C5═CC═C(F)C═C5 |
| Example 1231 | N1═C(SC═C1C(═O)NC(C)C)C═3OC(CN(CC═C)C2CCCCC2)═NC═3C4═CC═C(F)C═C4 |
| Example 1232 | N1═C(SC═C1C(═O)NC(C)C)C═3OC(CN2CCC(C(OC)═O)CC2)═NC═3C4═CC═C(F)C═C4 |
| Example 1233 | N1═C(SC═C1C(═O)NC(C)C)C═3OC(CN(C(C)C)CC2═CC═CC═C2)═NC═3C4═CC═C(F)C═C4 |
| Example 1234 | N1═C(SC═C1C(═O)NC(C)C)C═3OC(CN(CCO)CC2═CC═CC═C2)═NC═3C4═CC═C(F)C═C4 |
| Example 1235 | N1═C(SC═C1C(═O)NC(C)C)C═3OC(CN(C)CC(O)C2═CC═CC═C2)═NC═3C4═CC═C(F)C═C4 |
| Example 1236 | N1═C(SC═C1C(═O)NC(C)C)C═3OC(CN2CCC(O)(O)CC2)═NC═3C4═CC═C(F)C═C4 |
| Example 1237 | N1═C(SC═C1C(═O)NC(C)C)C═3OC(CN2CCC(C(OCC)═O)CC2)═NC═3C4═CC═C(F)C═C4 |
| Example 1238 | N1═C(SC═C1C(═O)NC(C)C)C═3OC(CN2CCCC(C(OCC)═O)C2)═NC═3C4═CC═C(F)C═C4 |
| Example 1239 | N1═C(SC═C1C(═O)NC(C)C)C═3OC(CN2CCN(C(OCC)═O)CC2)═NC═3C4═CC═C(F)C═C4 |
| Example 1240 | N1═C(SC═C1C(═O)NC(C)C)C═3OC(CN(CCC#N)CC2═CC═CN═C2)═NC═3C4═CC═C(F)C═C4 |
| Example 1241 | N1═C(SC═C1C(═O)NC(C)C)C═4OC(CN2CCN(CC2)C3═CC═CC═C3)═NC═4C5═CC═C(F)C═C5 |
| Example 1242 | N1═C(SC═C1C(═O)NC(C)C)C═4OC(CN2CCN(CC2)C3═CC═CC═N3)═NC═4C5═CC═C(F)C═C5 |
| Example 1243 | N1═C(SC═C1C(═O)NC(C)C)C═3OC(CN(CCCC)CC2═CC═CC═C2)═NC═3C4═CC═C(F)C═C4 |
| Example 1244 | N1═C(SC═C1C(═O)NC(C)C)C═3OC(CN([C@H](C)C2═CC═CC═C2)CCO)═NC═3C4═CC═C(F)C═C4 |
| Example 1245 | N1═C(SC═C1C(═O)NC(C)C)C═3OC(CN(CCCO)CC2═CC═CC═N2)═NC═3C4═CC═C(F)C═C4 |
| Example 1246 | N1═C(SC═C1C(═O)NC(C)C)C═3OC(CN(C)CC(O)C2═CC═C(O)C═C2)═NC═3C4═CC═C(F)C═C4 |
| Example 1247 | N1═C(SC═C1C(═O)NC(C)C)C═4OC(CN3CCN(C2CCCCC2)CC3)═NC═4C5═CC═C(F)C═C5 |
| Example 1248 | N1═C(SC═C1C(═O)NC(C)C)C═4OC(CN3CCC(CC2═CC═CC═C2)CC3)═NC═4C5═CC═C(F)C═C5 |
| Example 1249 | N1═C(SC═C1C(═O)NC(C)C)C═4OC(CN3CCN(CC2═CC═CC═C2)CC3)═NC═4C5═CC═C(F)C═C5 |
| Example 1250 | N1═C(SC═C1C(═O)NC(C)C)C═4OC(CN3CCCN(CC2═CC═CC═C2)CC3)═NC═4C5═CC═C(F)C═C5 |
| Example 1251 | N1═C(SC═C1C(═O)NC(C)C)C═4OC(CN3CCN(C2═CC═C(O)C═C2)CC3)═NC═4C5═CC═C(F)C═C5 |
| Example 1252 | N1═C(SC═C1C(═O)NC(C)C)C═3OC(CN(CCN(C)C)CC2═CC═CC═C2)═NC═3C4═CC═C(F)C═C4 |
| Example 1253 | N1═C(SC═C1C(═O)NC(C)C)C═4OC(CN3CCN(C2═CC═CC═C2F)CC3)═NC═4C5═CC═C(F)C═C5 |
| Example 1254 | N1═C(SC═C1C(═O)NC(C)C)C═4OC(CN3CCN(C2═CC═C(F)C═C2)CC3)═NC═4C5═CC═C(F)C═C5 |
| Example 1255 | N1═C(SC═C1C(═O)NC(C)C)C═4OC(CN3CCN(CC2CCCCC2)CC3)═NC═4C5═CC═C(F)C═C5 |
| Example 1256 | N1═C(SC═C1C(═O)NC(C)C)C═3OC(CN(C)C[C@H](O)C2═CC═C(O)C(O)═C2)═NC═3C4═CC═C(F)C═C4 |
| Example 1257 | N1═C(SC═C1C(═O)NC(C)C)C═3OC(CN2C(CC(OCC)═O)C(NCC2)═O)═NC═3C4═CC═C(F)C═C4 |
| Example 1258 | N1═C(SC═C1C(═O)NC(C)C)C═3OC(CN2CCN(C(OC(C)(C)C)═O)CC2)═NC═3C4═CC═C(F)C═C4 |
| Example 1259 | N1═C(SC═C1C(═O)NC(C)C)C═3OC(CN2CCC(NC(OC(C)(C)C)═O)C2)═NC═3C4═CC═C(F)C═C4 |
| Example 1260 | N1═C(SC═C1C(═O)NC(C)C)C═4OC(CN3CCN(C2═CC═CC═C2C#N)CC3)═NC═4C5═CC═C(F)C═C5 |
| Example 1261 | N1═C(SC═C1C(═O)NC(C)C)C═4OC(CN3CCN(C2═CC═C(C#N)C═N2)CC3═NC═4C5═CC═C(F)C═C5 |
| Example 1262 | N1═C(SC═C1C(═O)NC(C)C)C═4OC(CN3CCN(C═2C═CC═C(C)C═2C)CC3)═NC═4C5═CC═C(F)C═C5 |
| Example 1263 | N1═C(SC═C1C(═O)NC(C)C)C═4OC(CN3CCN(C═2C═CC═C(C)C═2C)CC3)═NC═4C5═CC═C(F)C═C5 |
| Example 1264 | N1═C(SC═C1C(═O)NC(C)C)C═4OC(CN2CC(C)N(CC2)C3═CC═CC(C)═C3)═NC═4C5═CC═C(F)C═C5 |
| Example 1265 | N1═C(SC═C1C(═O)NC(C)C)C═4OC(CN3CCN(CCC2═CC═CC═C2)CC3)═NC═4C5═CC═C(F)C═C5 |
| Example 1266 | N1═C(SC═C1C(═O)NC(C)C)C═4OC(CN3CCN(C2═CC═C(C)C(═C2)C)CC3)═NC═4C5═CC═C(F)C═C5 |
| Example 1267 | N1═C(SC═C1C(═O)NC(C)C)C═4OC(CN3CCN(C2═CC═C(C)C═C2C)CC3)═NC═4C5═CC═C(F)C═C5 |
| Example 1268 | N1═C(SC═C1C(═O)NC(C)C)C═4OC(CN3CCN(C═2C(═CC═C(C)C═2)C)CC3)═NC═4C5═CC═C(F)C═C5 |
| Example 1269 | N1═C(SC═C1C(═O)NC(C)C)C═4OC(CN3CCN(C2═CC═C(OC)C═C2)CC3)═NC═4C5═CC═C(F)C═C5 |
| Example 1270 | N1═C(SC═C1C(═O)NC(C)C)C═4OC(CN3CCN(C2═CC═CC═C2OC)CC3)═NC═4C5═CC═C(F)C═C5 |
| Example 1271 | N1═C(SC═C1C(═O)NC(C)C)C═4OC(CN3CCN(C═2C═CC═C(OC)C═2)CC3)═NC═4C5═CC═C(F)C═C5 |
| Example 1272 | N1═C(SC═C1C(═O)NC(C)C)C═3OC(CN(C)CCC2═CC═C(OC)C(OC)═C2)═NC═3C4═CC═C(F)C═C4 |
| Example 1273 | N1═C(SC═C1C(═O)NC(C)C)C═4OC(CN3CCN(C═2C═CC═C(Cl)C═2)CC3)═NC═4C5═CC═C(F)C═C5 |
| Example 1274 | N1═C(SC═C1C(═O)NC(C)C)C═4OC(CN3CCN(C2═CC═C(Cl)C═C2)CC3)═NC═4C5═CC═C(F)C═C5 |
| Example 1275 | N1═C(SC═C1C(═O)NC(C)C)C═4OC(CN3CCN(C2═CC═C(Cl)C═C2)CC3)═NC═4C5═CC═C(F)C═C5 |
| Example 1276 | N1═C(SC═C1C(═O)NC(C)C)C═4OC(CN3CCN(C2═CC═C(F)C═C2F)CC3)═NC═4C5═CC═C(F)C═C5 |
| Example 1277 | N1═C(SC═C1C(═O)NC(C)C)C═4OC(CN(CC2═CC═CN═C2)CC3═CC═CN═C3)═NC═4C5═CC═C(F)C═C5 |
| Example 1278 | N1═C(SC═C1C(═O)NC(C)C)C═4OC(CN3CCN(CCN2CCOCC2)CC3)═NC═4C5═CC═C(F)C═C5 |
| Example 1279 | N1═C(SC═C1C(═O)NC(C)C)C═3OC(CN2CCC(N(C)C(═O)OC(C)(C)C)C2)═NC═3C4═CC═C(F)C═C4 |
| Example 1280 | N1═C(SC═C1C(═O)NC(C)C)C═4OC(CN3CCN(C2═CC═C(C(C)═O)C═C2)CC3)═NC═4C5═CC═C(F)C═C5 |
| Example 1281 | N1═C(SC═C1C(═O)NC(C)C)C═3OC(CN(CCN(CC)CC)CC2═CC═CC═C2)═NC═3C4═CC═C(F)C═C4 |
| Example 1282 | N1═C(SC═C1C(═O)NC(C)C)C═4OC(CN3CCN(C2═CC═C([N+]([O−]═O)C═C2)CC3)═NC═4C5═CC═C(F)C═C5 |
| Example 1283 | N1═C(SC═C1C(═O)NC(C)C)C═4OC(CN(C)CC2═CC═CC3═CC═CC═C23)═NC═4C5═CC═C(F)C═C5 |
| Example 1284 | N1═C(SC═C1C(═O)NC(C)C)C═4OC(CN3CCN(C2═CC(═CC═C2C)Cl)CC3)═NC═4C5═CC═C(F)C═C5 |
| Example 1285 | N1═C(SC═C1C(═O)NC(C)C)C═3OC(CN(C)CC2═CC(OC)═C(OC)C(OC)═C2)═NC═3C4═CC═C(F)C═C4 |
| Example 1286 | N1═C(SC═C1C(═O)NC(C)C)C═4OC(CN(CCC2═CC═CC═C2)CC3═CC═CC═C3)═NC═4C5═CC═C(F)C═C5 |
| Example 1287 | N1═C(SC═C1C(═O)NC(C)C)C═4OC(CN(CCC2═CC═CC═C2)CC3═CC═CC═C3)═NC═4C5═CC═C(F)C═C5 |
| Example 1288 | N1═C(SC═C1C(═O)NC(C)C)C═4OC(CN(C)C(CC2═CC═CC═C2)C3═CC═CC═C3)═NC═4C5═CC═C(F)C═C5 |
| Example 1289 | N1═C(SC═C1C(═O)NC(C)C)C═4OC(CN2CCC(O)(CC2)C3═CC═C(Cl)C═C3)═NC═4C5═CC═C(F)C═C5 |
| Example 1290 | N1═C(SC═C1C(═O)NC(C)C)C═3OC(CN(CC2═CC═C(Cl)C═C2Cl)CC#C)═NC═3C4═CC═C(F)C═C4 |
| Example 1291 | N1═C(SC═C1C(═O)NC(C)C)C═4OC(CN(CC)C(CC2═CC═CC═C2)C═3OC═CC═3)═NC═4C5═CC═C(F)C═C5 |
| Example 1292 | N1═C(SC═C1C(═O)NC(C)C)C═3OC(CN2CCN(C(OC(C)(C)C)═O)C[C@H]2CO)═NC═3C4═CC═C(F)C═C4 |
| Example 1293 | N1═C(SC═C1C(═O)NC(C)C)C═4OC(CN3CCN(C2═CC═C(C(C)(C)C)C═C2)CC3)═NC═4C5═CC═C(F)C═C5 |
| Example 1294 | N1═C(SC═C1C(═O)NC(C)C)C═5OC(CN4CCN(CC3═CC═C2OCOC2═C3)CC4)═NC═5C6═CC═C(F)C═C6 |
| Example 1295 | N1═C(SC═C1C(═O)NC(C)C)C═4OC(CN2CCC═3C═C(OC)C(═CC═3C2)OC)═NC═4C5═CC═C(F)C═C5 |
| Example 1296 | N1═C(SC═C1C(═O)NC(C)C)C═4OC(CN3CCN(C2═CC═C(C(F)(F)F)C═C2)CC3)═NC═4C5═CC═C(F)C═C5 |
| Example 1297 | N1═C(SC═C1C(═O)NC(C)C)C═4OC(CN3CCN(C═2C═CC═C(C(F)(F)F)C═2)CC3)═NC═4C5═CC═C(F)C═C5 |
| Example 1298 | N1═C(SC═C1C(═O)NC(C)C)C═4OC(CN3CCN(C2═CC═C(Cl)C(═C2)Cl)CC3)═NC═4C5═CC═C(F)C═C5 |
| Example 1299 | N1═C(SC═C1C(═O)NC(C)C)C═4OC(CN3CCN(C2═CC═C(Cl)C(═C2)Cl)CC3)═NC═4C5═CC═C(F)C═C5 |
| Example 1300 | N1═C(SC═C1C(═O)NC(C)C)C═4OC(CN3CCN(C2═CC═C(C(F)(F)F)C═N2)CC3)═NC═4C5═CC═C(F)C═C5 |
| Example 1301 | N1═C(SC═C1C(═O)NC(C)C)C═4OC(CN3CCN(C2═CC═CC═C2Cl)CC3)═NC═4C5═CC═C(F)C═C5 |
| Example 1302 | N1═C(SC═C1C(═O)NC(C)C)C═4OC(CN3CCN(C2═CC═C(OC)C(═C2)OC)CC3)═NC═4C5═CC═C(F)C═C5 |
| Example 1303 | N1═C(SC═C1C(═O)NC(C)C)C═4OC(CN3CCCC(C2═CC═C(C(F)(F)F)C═C2)C3)═NC═4C5═CC═C(F)C═C5 |

Examples Prepared with Anilines:

| | |
|---|---|
| Example 1304 | N1═C(SC═C1C(═O)NC(C)C)C═4OC(CNC2CCCC3═CC═CC═C23)═NC═4C5═CC═C(F)C═C5 |
| Example 1305 | N1═C(SC═C1C(═O)NC(C)C)C═3OC(CNC2═CC═C(OC)C(═C2)OC)═NC═3C4═CC═C(F)C═C4 |
| Example 1306 | N1═C(SC═C1C(═O)NC(C)C)C═2OC(CBr)═NC═2C3═CC═C(F)C═C3 |
| Example 1307 | N1═C(SC═C1C(═O)NC(C)C)C═3OC(CNC2═CC═CC═C2)═NC═3C4═CC═C(F)C═C4 |
| Example 1308 | N1═C(SC═C1C(═O)NC(C)C)C═3OC(CNC═2C═CC═C(C)C═2)═NC═3C4═CC═C(F)C═C4 |
| Example 1309 | N1═C(SC═C1C(═O)NC(C)C)C═3OC(CNC2═CC═C(C)C═C2)═NC═3C4═CC═C(F)C═C4 |
| Example 1310 | N1═C(SC═C1C(═O)NC(C)C)C═3OC(CNC2═CC═CC═C2C)═NC═3C4═CC═C(F)C═C4 |
| Example 1311 | N1═C(SC═C1C(═O)NC(C)C)C═3OC(CNC2═CC═C(C)C═N2)═NC═3C4═CC═C(F)C═C4 |
| Example 1312 | N1═C(SC═C1C(═O)NC(C)C)C═3OC(CNC═2C═CC═C(C)N═2)═NC═3C4═CC═C(F)C═C4 |
| Example 1313 | N1═C(SC═C1C(═O)NC(C)C)C═3OC(CNC═2N═CC═C(C)C═2)═NC═3C4═CC═C(F)C═C4 |
| Example 1314 | N1═C(SC═C1C(═O)NC(C)C)C═3OC(CNC2═NC═CC═C2C)═NC═3C4═CC═C(F)C═C4 |
| Example 1315 | N1═C(SC═C1C(═O)NC(C)C)C═3OC(CNC2═CC═CC═C2F)═NC═3C4═CC═C(F)C═C4 |
| Example 1316 | N1═C(SC═C1C(═O)NC(C)C)C═3OC(CNC2═CC═C(F)C═C2)═NC═3C4═CC═C(F)C═C4 |
| Example 1317 | N1═C(SC═C1C(═O)NC(C)C)C═3OC(CNC═2C(═CC═C(C)C═2)C)═NC═3C4═CC═C(F)C═C4 |
| Example 1318 | N1═C(SC═C1C(═O)NC(C)C)C═3OC(CNC2═CC═C(C)C(═C2)C)═NC═3C4═CC═C(F)C═C4 |
| Example 1319 | N1═C(SC═C1C(═O)NC(C)C)C═3OC(CNC2═CC═CC═C2CC)═NC═3C4═CC═C(F)C═C4 |
| Example 1320 | N1═C(SC═C1C(═O)NC(C)C)C═3OC(CNC2═CC═CC═C2CC)═NC═3C4═CC═C(F)C═C4 |
| Example 1321 | N1═C(SC═C1C(═O)NC(C)C)C═3OC(CNC═2C═CC═C(C)C═2C)═NC═3C4═CC═C(F)C═C4 |
| Example 1322 | N1═C(SC═C1C(═O)NC(C)C)C═3OC(CNC2═CC═C(C)C═C2C)═NC═3C4═CC═C(F)C═C4 |
| Example 1323 | N1═C(SC═C1C(═O)NC(C)C)C═3OC(CNC═2C═C(C═C(C)C═2)C)═NC═3C4═CC═C(F)C═C4 |
| Example 1324 | N1═C(SC═C1C(═O)NC(C)C)C═3OC(CNC═2C═CC═C(OC)C═2)═NC═3C4═CC═C(F)C═C4 |
| Example 1325 | N1═C(SC═C1C(═O)NC(C)C)C═3OC(CNC2═CC═C(OC)C═C2)═NC═3C4═CC═C(F)C═C4 |
| Example 1326 | N1═C(SC═C1C(═O)NC(C)C)C═3OC(CNC2═CC═CC═C2OC)═NC═3C4═CC═C(F)C═C4 |
| Example 1327 | N1═C(SC═C1C(═O)NC(C)C)C═3OC(CNC2═CN═CC═C2OC)═NC═3C4═CC═C(F)C═C4 |
| Example 1328 | N1═C(SC═C1C(═O)NC(C)C)C═3OC(CNC2═CC═C(C)C(═C2)F)═NC═3C4═CC═C(F)C═C4 |
| Example 1329 | N1═C(SC═C1C(═O)NC(C)C)C═3OC(CNC2═C(C)C(═CC═C2)F)═NC═3C4═CC═C(F)C═C4 |
| Example 1330 | N1═C(SC═C1C(═O)NC(C)C)C═3OC(CNC2═CC(F)═CC(F)═C2)═NC═3C4═CC═C(F)C═C4 |
| Example 1331 | N1═C(SC═C1C(═O)NC(C)C)C═3OC(CNC2═CC═C(F)C═C2F)═NC═3C4═CC═C(F)C═C4 |
| Example 1332 | N1═C(SC═C1C(═O)NC(C)C)C═3OC(CNC2═CC═C(F)C═C2F)═NC═3C4═CC═C(F)C═C4 |
| Example 1333 | N1═C(SC═C1C(═O)NC(C)C)C═4OC(CNC3═CC═C2CCCC2═C3)═NC═4C5═CC═C(F)C═C5 |
| Example 1334 | N1═C(SC═C1C(═O)NC(C)C)C═3OC(CNC2═CC═CC═C2C(C)C)═NC═3C4═CC═C(F)C═C4 |
| Example 1335 | N1═C(SC═C1C(═O)NC(C)C)C═3OC(CNC═2C═CC═C(C(C)═O)C═2)═NC═3C4═CC═C(F)C═C4 |
| Example 1336 | N1═C(SC═C1C(═O)NC(C)C)C═3OC(CNC2═CC═C(C(C)═O)C═C2)═NC═3C4═CC═C(F)C═C4 |
| Example 1337 | N1═C(SC═C1C(═O)NC(C)C)C═3OC(CNC2═C(C═CC═C2CC)C)═NC═3C4═CC═C(F)C═C4 |
| Example 1338 | N1═C(SC═C1C(═O)NC(C)C)C═3OC(CNC2═C(C═C(C)C═C2C)C)═NC═3C4═CC═C(F)C═C4 |
| Example 1339 | N1═C(SC═C1C(═O)NC(C)C)C═4OC(CNC3═CC═C2OCOC2═C3)═NC═4C5═CC═C(F)C═C5 |
| Example 1340 | N1═C(SC═C1C(═O)NC(C)C)C═3OC(CNC2═CC═C(OC)C═C2C)═NC═3C4═CC═C(F)C═C4 |
| Example 1341 | N1═C(SC═C1C(═O)NC(C)C)C═3OC(CNC═2C═CC═C(OCC)C═2)═NC═3C4═CC═C(F)C═C4 |
| Example 1342 | N1═C(SC═C1C(═O)NC(C)C)C═3OC(CNC2═CC═CC═C2OCC)═NC═3C4═CC═C(F)C═C4 |
| Example 1343 | N1═C(SC═C1C(═O)NC(C)C)C═3OC(CNC2═CC═CC═C2SC)═NC═3C4═CC═C(F)C═C4 |
| Example 1344 | N1═C(SC═C1C(═O)NC(C)C)C═3OC(CNC2═C(C)C(═CC═C2)Cl)═NC═3C4═CC═C(F)C═C4 |
| Example 1345 | N1═C(SC═C1C(═O)NC(C)C)C═3OC(CNC2═CC═C(C)C(═C2)Cl)═NC═3C4═CC═C(F)C═C4 |
| Example 1346 | N1═C(SC═C1C(═O)NC(C)C)C═4OC(CNC3═CN═C2C═CC═CC2═C3)═NC═4C5═CC═C(F)C═C5 |
| Example 1347 | N1═C(SC═C1C(═O)NC(C)C)C═3OC(CNC2═CC(F)═C(F)C═C2F)═NC═3C4═CC═C(F)C═C4 |
| Example 1348 | N1═C(SC═C1C(═O)NC(C)C)C═3OC(CNC2═CC═CC═C2C(C)(C)C)═NC═3C4═CC═C(F)C═C4 |
| Example 1349 | N1═C(SC═C1C(═O)NC(C)C)C═3OC(CNC2═C(C═CC═C2C(C)C)C)═NC═3C4═CC═C(F)C═C4 |
| Example 1350 | N1═C(SC═C1C(═O)NC(C)C)C═4OC(CNC3═CC═C2N═CSC2═C3)═NC═4C5═CC═C(F)C═C5 |
| Example 1351 | N1═C(SC═C1C(═O)NC(C)C)C═3OC(CNC2═CC═C(NC(C)═O)C═C2)═NC═3C4═CC═C(F)C═C4 |
| Example 1352 | N1═C(SC═C1C(═O)NC(C)C)C═3OC(CNC2═CC═C(NC(C)═O)N═C2)═NC═3C4═CC═C(F)C═C4 |
| Example 1353 | N1═C(SC═C1C(═O)NC(C)C)C═4OC(CNC3═CC═C2OCCOC2═C3)═NC═4C5═CC═C(F)C═C5 |
| Example 1354 | N1═C(SC═C1C(═O)NC(C)C)C═4OC(CNC2═CC═CC═C2N3C═CC═C3)═NC═4C5═CC═C(F)C═C5 |
| Example 1355 | N1═C(SC═C1C(═O)NC(C)C)C═3OC(CNC2═CC═C(C═C2)C(F)(F)F)═NC═3C4═CC═C(F)C═C4 |
| Example 1356 | N1═C(SC═C1C(═O)NC(C)C)C═3OC(CNC2═CC═CC(═C2)C(F)(F)F)═NC═3C4═CC═C(F)C═C4 |
| Example 1357 | N1═C(SC═C1C(═O)NC(C)C)C═3OC(CNC2═CC═C(Cl)C(Cl)═C2)═NC═3C4═CC═C(F)C═C4 |
| Example 1358 | N1═C(SC═C1C(═O)NC(C)C)C═3OC(CNC2═C(C═CC═C2C)C(C)(C)C)═NC═3C4═CC═C(F)C═C4 |
| Example 1359 | N1═C(SC═C1C(═O)NC(C)C)C═3OC(CNC2═CC═C(N(C)C)C═C2)═NC═3C4═CC═C(F)C═C4 |
| Example 1360 | N1═C(SC═C1C(═O)NC(C)C)C═4OC(CNC2═CC═CC═C2C3═CC═CC═C3)═NC═4C5═CC═C(F)C═C5 |
| Example 1361 | N1═C(SC═C1C(═O)NC(C)C)C═3OC(CNC2═CC═C(OC(F)(F)F)C═C2)═NC═3C4═CC═C(F)C═C4 |
| Example 1362 | N1═C(SC═C1C(═O)NC(C)C)C═3OC(CNC2═C(C═CC═C2C(C)C)C(C)C)═NC═3C4═CC═C(F)C═C4 |
| Example 1363 | N1═C(SC═C1C(═O)NC(C)C)C═3OC(CNC2═CC═C(F)C═C2C(F)(F)F)═NC═3C4═CC═C(F)C═C4 |
| Example 1364 | N1═C(SC═C1C(═O)NC(C)C)C═3OC(CNC2═CC(═CC═C2F)C(F)(F)F)═NC═3C4═CC═C(F)C═C4 |
| Example 1365 | N1═C(SC═C1C(═O)NC(C)C)C═3OC(CNC2═CC═C(F)C(═C2)C(F)(F)F)═NC═3C4═CC═C(F)C═C4 |
| Example 1366 | N1═C(SC═C1C(═O)NC(C)C)C═3OC(CNC═2C(═CC═C(OCC)C═2)OCC)═NC═3C4═CC═C(F)C═C4 |
| Example 1367 | N1═C(SC═C1C(═O)NC(C)C)C═3OC(CNC2═CC(Cl)═C(Cl)C(Cl)═C2)═NC═3C4═CC═C(F)C═C4 |
| Example 1368 | N1═C(SC═C1C(═O)NC(C)C)C═4OC(CNC2═CC═CC═C2C(═O)C3═CC═CC═C3)═NC═4C5═CC═C(F)C═C5 |
| Example 1369 | N1═C(SC═C1C(═O)NC(C)C)C═4OC(CNC2═CC═CC═C2COC3═CC═CC═C3)═NC═4C5═CC═C(F)C═C5 |
| Example 1370 | N1═C(SC═C1C(═O)NC(C)C)C═4OC(CNC2═CC(═CC═C2OC)C3═CC═CC═C3)═NC═4C5═CC═C(F)C═C5 |
| Example 1371 | N1═C(SC═C1C(═O)NC(C)C)C═4OC(CNC2═NC═CC═C2OCC3═CC═CC═C3)═NC═4C5═CC═C(F)C═C5 |
| Example 1372 | N1═C(SC═C1C(═O)NC(C)C)C═3OC(CNC2═CC(═CC═C2C(F)(F)F)C(F)(F)F)═NC═3C4═CC═C(F)C═C4 |
| Example 1373 | N1═C(SC═C1C(═O)NC(C)C)C═3OC(CNC2═CC(═CC(═C2)C(F)(F)F)C(F)(F)F)═NC═3C4═CC═C(F)C═C4 |
| Example 1374 | N1═C(SC═C1C(═O)NC(C)C)C═3OC(CNC2═CC═C(C═C2C(F)(F)F)C(F)(F)F)═NC═3C4═CC═C(F)C═C4 |

### EXAMPLES 1375-1700

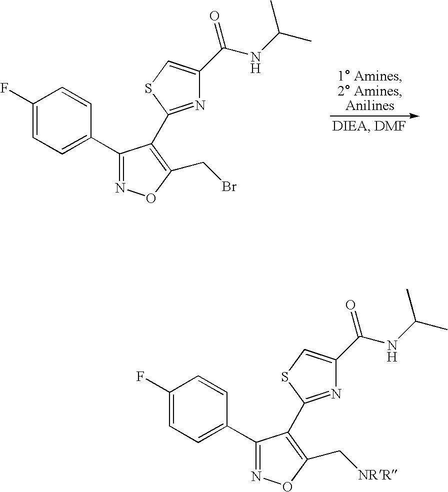 Starting bromomethyl isoxazole was prepared as described in Step 2 of Example 28. Where, 1° amines, 2° amines, and anilines were selected to afford Examples 1374-1700, which were prepared by General Procedure 2.

Examples Prepared with 1° Amines:

| | |
|---|---|
| Example 1375 | OCCNCC2═C(C(C1═CC═C(C═C1)F)═NO2)C3═NC(═CS3)C(NC(C)C)═O |
| Example 1376 | CCOC(N4CCC(NCC2═C(C(C1═CC═C(C═C1)F)═NO2)C3═NC(═CS3)C(NC(C)C)═O)CC4)═O |
| Example 1377 | COCCNCC2═C(C(C1═CC═C(C═C1)F)═NO2)C3═NC(═CS3)C(NC(C)C)═O |
| Example 1378 | C1═CC(═CC═C1F)C4═NOC(CNC2CCCC3═CC═CC═C23)═C4C5═NC(C(NC(C)C)═O)═CS5 |
| Example 1379 | C1═CC(═CC═C1F)C3═NOC(CNC2═CC═C(OC)C(═C2)OC)═C3C4═NC(C(NC(C)C)═O)═CS4 |
| Example 1380 | C1═CC(═CC═C1F)C2═NOC(CBr)═C2C3═NC(C(N([H])C(C)C)═O)═CS3 |
| Example 1381 | C1═CC(═CC═C1F)C3═NOC(CN(CCC#N)CC2═CC═CC═C2)═C3C4═NC(C(NC(C)C)═O)═CS4 |
| Example 1382 | C1═CC(═CC═C1F)C2═NOC(CNC)═C2C3═NC(C(NC(C)C)═O)═CS3 |
| Example 1383 | C1═CC(═CC═C1F)C2═NOC(CNCC)═C2C3═NC(C(NC(C)C)═O)═CS3 |
| Example 1384 | C1═CC(═CC═C1F)C2═NOC(CNCC#C)═C2C3═NC(C(NC(C)C)═O)═CS3 |
| Example 1385 | C1═CC(═CC═C1F)C2═NOC(CNCC#N)═C2C3═NC(C(NC(C)C)═O)═CS3 |
| Example 1386 | C1═CC(═CC═C1F)C3═NOC(CNC2CC2)═C3C4═NC(C(NC(C)C)═O)═CS4 |
| Example 1387 | C1═CC(═CC═C1F)C2═NOC(CNC(C)C)═C2C3═NC(C(NC(C)C)═O)═CS3 |
| Example 1388 | C1═CC(═CC═C1F)C2═NOC(CNCCC)═C2C3═NC(C(NC(C)C)═O)═CS3 |
| Example 1389 | C1═CC(═CC═C1F)C2═NOC(CNCCN)═C2C3═NC(C(NC(C)C)═O)═CS3 |
| Example 1390 | C1═CC(═CC═C1F)C2═NOC(CNCCC#N)═C2C3═NC(C(NC(C)C)═O)═CS3 |
| Example 1391 | C1═CC(═CC═C1F)C3═NOC(CNC2CCC2)═C3C4═NC(C(NC(C)C)═O)═CS4 |
| Example 1392 | C1═CC(═CC═C1F)C3═NOC(CNCC2CC2)═C3C4═NC(C(NC(C)C)═O)═CS4 |
| Example 1393 | C1═CC(═CC═C1F)C2═NOC(CNC(CC)C)═C2C3═NC(C(NC(C)C)═O)═CS3 |
| Example 1394 | C1═CC(═CC═C1F)C2═NOC(CNCC(C)C)═C2C3═NC(C(NC(C)C)═O)═CS3 |
| Example 1395 | C1═CC(═CC═C1F)C2═NOC(CNCCCO)═C2C3═NC(C(NC(C)C)═O)═CS3 |
| Example 1396 | C1═CC(═CC═C1F)C2═NOC(CNC(C)CO)═C2C3═NC(C(NC(C)C)═O)═CS3 |
| Example 1397 | C1═CC(═CC═C1F)C3═NOC(CNC2CCCC2)═C3C4═NC(C(NC(C)C)═O)═CS4 |
| Example 1398 | C1═CC(═CC═C1F)C2═NOC(CNC(CCC)C)═C2C3═NC(C(NC(C)C)═O)═CS3 |
| Example 1399 | C1═CC(═CC═C1F)C2═NOC(CNCC(CC)C)═C2C3═NC(C(NC(C)C)═O)═CS3 |
| Example 1400 | C1═CC(═CC═C1F)C2═NOC(CNC(C(C)C)C)═C2C3═NC(C(NC(C)C)═O)═CS3 |
| Example 1401 | C1═CC(═CC═C1F)C2═NOC(CNCCC(C)C)═C2C3═NC(C(NC(C)C)═O)═CS3 |
| Example 1402 | C1═CC(═CC═C1F)C2═NOC(CNCCN(C)C)═C2C3═NC(C(NC(C)C)═O)═CS3 |
| Example 1403 | C1═CC(═CC═C1F)C2═NOC(CNC(COC)C)═C2C3═NC(C(NC(C)C)═O)═CS3 |
| Example 1404 | C1═CC(═CC═C1F)C2═NOC(CNC)(CC)CO)═C2C3═NC(C(NC(C)C)═O)═CS3 |
| Example 1405 | C1═CC(═CC═C1F)C2═NOC(CNC(CC)CO)═C2C3═NC(C(NC(C)C)═O)═CS3 |
| Example 1406 | C1═CC(═CC═C1F)C2═NOC(CNCCCCO)═C2C3═NC(C(NC(C)C)═O)═CS3 |
| Example 1407 | C1═CC(═CC═C1F)C3═NOC(CNCC═2OC═CC═2)═C3C4═NC(C(NC(C)C)═O)═CS4 |
| Example 1408 | C1═CC(═CC═C1F)C3═NOC(CNC2CCCCC2)═C3C4═NC(C(NC(C)C)═O)═CS4 |
| Example 1409 | C1═CC(═CC═C1F)C3═NOC(CNCC2CCCO2)═C3C4═NC(C(NC(C)C)═O)═CS4 |
| Example 1410 | C1═CC(═CC═C1F)C2═NOC(CNCCC(C)(C)C)═C2C3═NC(C(NC(C)C)═O)═CS3 |
| Example 1411 | C1═CC(═CC═C1F)C2═NOC(CNC(CC(C)C)C)═C2C3═NC(C(NC(C)C)═O)═CS3 |
| Example 1412 | C1═CC(═CC═C1F)C3═NOC(CNC2CONC2═O)═C3C4═NC(C(NC(C)C)═O)═CS4 |
| Example 1413 | C1═CC(═CC═C1F)C3═NOC(CNN2CCOCC2)═C3C4═NC(C(NC(C)C)═O)═CS4 |
| Example 1414 | C1═CC(═CC═C1F)C2═NOC(CNC(C(C)C)CO)═C2C3═NC(C(NC(C)C)═O)═CS3 |
| Example 1415 | C1═CC(═CC═C1F)C3═NOC(CNCC2═CC═CC═C2)═C3C4═NC(C(NC(C)C)═O)═CS4 |
| Example 1416 | C1═CC(═CC═C1F)C3═NOC(CNCC2═CC═CN═C2)═C3C4═NC(C(NC(C)C)═O)═CS4 |
| Example 1417 | C1═CC(═CC═C1F)C3═NOC(CNCC2═CC═CC═N2)═C3C4═NC(C(NC(C)C)═O)═CS4 |
| Example 1418 | C1═CC(═CC═C1F)C3═NOC(CNCC2═CC═NC═C2)═C3C4═NC(C(NC(C)C)═O)═CS4 |
| Example 1419 | C1═CC(═CC═C1F)C3═NOC(CNCC2═CC═CS2═C3C4═NC(C(NC(C)C)═O)═CS4 |
| Example 1420 | C1═CC(═CC═C1F)C3═NOC(CNC2CCC(C)CC2)═C3C4═NC(C(NC(C)C)═O)═CS4 |
| Example 1421 | C1═CC(═CC═C1F)C3═NOC(CNCC2CCCCC2)═C3C4═NC(C(NC(C)C)═O)═CS4 |
| Example 1422 | C1═CC(═CC═C1F)C3═NOC(CNC2CCCCCC2)═C3C4═NC(C(NC(C)C)═O)═CS4 |
| Example 1423 | C1═CC(═CC═C1F)C3═NOC(CNC2CCCCC2C)═C3C4═NC(C(NC(C)C)═O)═CS4 |
| Example 1424 | C1═CC(═CC═C1F)C3═NOC(CNCCN2CCCC2)═C3C4═NC(C(NC(C)C)═O)═CS4 |
| Example 1425 | C1═CC(═CC═C1F)C3═NOC(CNN2CCN(C)CC2)═C3C4═NC(C(NC(C)C)═O)═CS4 |
| Example 1426 | C1═CC(═CC═C1F)C2═NOC(CNCCN(CC)CC)═C2C3═NC(C(NC(C)C)═O)═CS3 |
| Example 1427 | C1═CC(═CC═C1F)C3═NOC(CNCC═2C═CC═C(C)C═2)═C3C4═NC(C(NC(C)C)═O)═CS4 |
| Example 1428 | C1═CC(═CC═C1F)C3═NOC(CN[C@@H](C)C2═CC═CC═C2)═C3C4═NC(C(NC(C)C)═O)═CS4 |
| Example 1429 | C1═CC(═CC═C1F)C3═NOC(CN[C@H](C)C2═CC═CC═C2)═C3C4═NC(C(NC(C)C)═O)═CS4 |
| Example 1430 | C1═CC(═CC═C1F)C3═NOC(CNCCC2═CC═CC═C2)═C3C4═NC(C(NC(C)C)═O)═CS4 |
| Example 1431 | C1═CC(═CC═C1F)C3═NOC(CNCC2═CC═CC═C2C)═C3C4═NC(C(NC(C)C)═O)═CS4 |
| Example 1432 | C1═CC(═CC═C1F)C3═NOC(CNCC2═CC═C(C)C═C2)═C3C4═NC(C(NC(C)C)═O)═CS4 |
| Example 1433 | C1═CC(═CC═C1F)C3═NOC(CNCC2═NC═C(C)N═C2)═C3C4═NC(C(NC(C)C)═O)═CS4 |
| Example 1434 | C1═CC(═CC═C1F)C3═NOC(CNCC2═CC═CC(F)═C2)═C3C4═NC(C(NC(C)C)═O)═CS4 |
| Example 1435 | C1═CC(═CC═C1F)C3═NOC(CNCC2═CC═C(F)C═C2)═C3C4═NC(C(NC(C)C)═O)═CS4 |
| Example 1436 | C1═CC(═CC═C1F)C3═NOC(CNCC2═CC═CC═C2F)═C3C4═NC(C(NC(C)C)═O)═CS4 |
| Example 1437 | C1═CC(═CC═C1F)C3═NOC(CNCCCN2C═CN═C2)═C3C4═NC(C(NC(C)C)═O)═CS4 |
| Example 1438 | C1═CC(═CC═C1F)C3═NOC(CNCCC2CCCN2C)═C3C4═NC(C(NC(C)C)═O)═CS4 |
| Example 1439 | C1═CC(═CC═C1F)C3═NOC(CNCCN2CCCCC2)═C3C4═NC(C(NC(C)C)═O)═CS4 |
| Example 1440 | C1═CC(═CC═C1F)C2═NOC(CNC(CCCC(C)C)C)═C2C3═NC(C(NC(C)C)═O)═CS3 |
| Example 1441 | C1═CC(═CC═C1F)C3═NOC(CNCCN2CCOCC2)═C3C4═NC(C(NC(C)C)═O)═CS4 |
| Example 1442 | C1═CC(═CC═C1F)C2═NOC(CNC(CC(OCC)═O)C)═C2C3═NC(C(NC(C)C)═O)═CS3 |
| Example 1443 | C1═CC(═CC═C1F)C4═NOC(CNC2CCC3═CC═CC═C23)═C4C5═NC(C(NC(C)C)═O)═CS5 |
| Example 1444 | C1═CC(═CC═C1F)C3═NOC(CNCCC2═CC═C(C)C═C2)═C3C4═NC(C(NC(C)C)═O)═CS4 |
| Example 1445 | C1═CC(═CC═C1F)C3═NOC(CNCC═2C(═CC═C(C)C═2)C)═C3C4═NC(C(NC(C)C)═O)═CS4 |
| Example 1446 | C1═CC(═CC═C1F)C3═NOC(CN[C@@H](C)C2═CC═C(C)C═C2)═C3C4═NC(C(NC(C)C)═O)═CS4 |
| Example 1447 | C1═CC(═CC═C1F)C3═NOC(CN[C@H](C)C2═CC═C(C)C═C2)═C3C4═NC(C(NC(C)C)═O)═CS4 |
| Example 1448 | C1═CC(═CC═C1F)C3═NOC(CNCC(C)C2═CC═CC═C2)═C3C4═NC(C(NC(C)C)═O)═CS4 |
| Example 1449 | C1═CC(═CC═C1F)C3═NOC(CNCC(C)C2═CC═CC═C2)═C3C4═NC(C(NC(C)C)═O)═CS4 |
| Example 1450 | C1═CC(═CC═C1F)C3═NOC(CNCCCC2═CC═CC═C2)═C3C4═NC(C(NC(C)C)═O)═CS4 |
| Example 1451 | C1═CC(═CC═C1F)C3═NOC(CNCC2═CC═C(C)C(═C2)C)═C3C4═NC(C(NC(C)C)═O)═CS4 |
| Example 1452 | C1═CC(═CC═C1F)C3═NOC(CNC(C)CC2═CC═NC═C2)═C3C4═NC(C(NC(C)C)═O)═CS4 |
| Example 1453 | C1═CC(═CC═C1F)C3═NOC(CNCCC2═CC═C(O)C═C2)═C3C4═NC(C(NC(C)C)═O)═CS4 |
| Example 1454 | C1═CC(═CC═C1F)C3═NOC(CNCCOC2═CC═CC═C2)═C3C4═NC(C(NC(C)C)═O)═CS4 |
| Example 1455 | C1═CC(═CC═C1F)C3═NOC(CNCC2═CC═CC═C2OC)═C3C4═NC(C(NC(C)C)═O)═CS4 |
| Example 1456 | C1═CC(═CC═C1F)C3═NOC(CNCC2═CC═C(OC)C═C2)═C3C4═NC(C(NC(C)C)═O)═CS4 |
| Example 1457 | C1═CC(═CC═C1F)C3═NOC(CNCC═2C═CC═C(OC)C═2)═C3C4═NC(C(NCC(C)C)═O)═CS4 |
| Example 1458 | C1═CC(═CC═C1F)C3═NOC(CNCCC2═CC═C(F)C═C2)═C3C4═NC(C(NC(C)C)═O)═CS4 |
| Example 1459 | C1═CC(═CC═C1F)C3═NOC(CNCCC2═CC═CC═C2F)═C3C4═NC(C(NC(C)C)═O)═CS4 |
| Example 1460 | C1═CC(═CC═C1F)C3═NOC(CNCCC2═CC═CC(F)═C2)═C3C4═NC(C(NC(C)C)═O)═CS4 |
| Example 1461 | C1═CC(═CC═C1F)C3═NOC(CNCC2═CC═CC(Cl)═C2)═C3C4═NC(C(NC(C)C)═O)═CS4 |
| Example 1462 | C1═CC(═CC═C1F)C3═NOC(CNCC2═CC═CC═C2Cl)═C3C4═NC(C(NC(C)C)═O)═CS4 |
| Example 1463 | C1═CC(═CC═C1F)C3═NOC(CNCC2═CC═C(Cl)C═C2)═C3C4═NC(C(NC(C)C)═O)═CS4 |
| Example 1464 | C1═CC(═CC═C1F)C3═NOC(CNCCCN2CCCC2═O)═C3C4═NC(C(NC(C)C)═O)═CS4 |
| Example 1465 | C1═CC(═CC═C1F)C3═NOC(CNCC2═CC═C(F)C═C2F)═C3C4═NC(C(NC(C)C)═O)═CS4 |
| Example 1466 | C1═CC(═CC═C1F)C3═NOC(CNCC2═CC═C(F)C(F)═C2)═C3C4═NC(C(NC(C)C)═O)═CS4 |
| Example 1467 | C1═CC(═CC═C1F)C3═NOC(CNCC2═CC(F)═CC(F)═C2)═C3C4═NC(C(NC(C)C)═O)═CS4 |
| Example 1468 | C1═CC(═CC═C1F)C3═NOC(CNCCCN2CCOCC2)═C3C4═NC(C(NC(C)C)═O)═CS4 |
| Example 1469 | C1═CC(═CC═C1F)C2═NOC(CNCCN(C(C)C)C(C)C)═C2C3═NC(C(NC(C)C)═O)═CS3 |
| Example 1470 | C1═CC(═CC═C1F)C3═NOC(CNC(C)CCC2═CC═CC═C2)═C3C4═NC(C(NC(C)C)═O)═CS4 |
| Example 1471 | C1═CC(═CC═C1F)C3═NOC(CNCC2═CC═C(C(C)C)C═C2)═C3C4═NC(C(NC(C)C)═O)═CS4 |
| Example 1472 | C1═CC(═CC═C1 F)C4═NOC(CNCC3═CC═C2OCOC2═C3)═C4C5═NC(C(NC(C)C)═O)═CS5 |
| Example 1473 | C1═CC(═CC═C1F)C3═NOC(CNCCC═2C═CC═C(OC)C═2)═C3C4═NC(C(NC(C)C)═O)═CS4 |
| Example 1474 | C1═CC(═CC═C1F)C3═NOC(CN[C@H](CO)CC2═CC═CC═C2)═C3C4═NC(C(NC(C)C)═O)═CS4 |
| Example 1475 | C1═CC(═CC═C1F)C3═NOC(CNCCC2═CC═C(OC)C═C2)═C3C4═NC(C(NC(C)C)═O)═CS4 |
| Example 1476 | C1═CC(═CC═C1F)C3═NOC(CNCCC2═CC═CC═C2OC)═C3C4═NC(C(NC(C)C)═O)═CS4 |
| Example 1477 | C1═CC(═CC═C1F)C3═NOC(CNCC2═CC═CC═C2OCC)═C3C4═NC(C(NC(C)C)═O)═CS4 |
| Example 1478 | C1═CC(═CC═C1F)C3═NOC(CNCCC2═CC═CC═C2Cl)═C3C4═NC(C(NC(C)C)═O)═CS4 |
| Example 1479 | C1═CC(═CC═C1F)C3═NOC(CNCCC2═CC═C(Cl)C═C2)═C3C4═NC(C(NC(C)C)═O)═CS4 |
| Example 1480 | C1═CC(═CC═C1F)C3═NOC(CNCCC2═CC═CC(Cl)═C2)═C3C4═NC(C(NC(C)C)═O)═CS4 |
| Example 1481 | C1═CC(═CC═C1F)C3═NOC(CNC2CC(C)(C)NC(C2)(C)C)═C3C4═NC(C(NC(C)C)═O)═CS4 |
| Example 1482 | C1═CC(═CC═C1F)C2═NOC(CNC(CCCN(CC)CC)C)═C2C3═NC(C(NC(C)C)═O)═CS3 |
| Example 1483 | C1═CC(═CC═C1F)C3═NOC(CNCC2═CC═C(F)C(Cl)═C2)═C3C4═NC(C(NC(C)C)═O)═CS4 |
| Example 1484 | C1═CC(═CC═C1F)C2═NOC(CNCCNC(OC(C)(C)C)═O)═C2C3═NC(C(NC(C)C)═O)═CS3 |
| Example 1485 | [H]N4C═C(CCNCC═2ON═C(C1═CC═C(F)C═C1)C═2C3═NC(C(NC(C)C)═O)═CS3)C5═CC═CC═C45 |
| Example 1486 | C1═CC(═CC═C1F)C3═NOC(CNCC2═CC═C(C(C)(C)C)C═C2)═C3C4═NC(C(NC(C)C)═O)═CS4 |
| Example 1487 | C1═CC(═CC═C1F)C3═NOC(CNCCCN(C)C2═CC═CC═C2)═C3C4═NC(C(NC(C)C)═O)═CS4 |
| Example 1488 | C1═CC(═CC═C1F)C3═NOC(CNCC2(O)CCCCC2)═C3C4═NC(C(NC(C)C)═O)═CS4 |
| Example 1489 | C1═CC(═CC═C1F)C3═NOC(CNCC═2C═C(OC)C═C(C═2)OC)═C3C4═NC(C(NC(C)C)═O)═CS4 |
| Example 1490 | C1═CC(═CC═C1F)C3═NOC(CNCC2═CC═C(OC)C(═C2)OC)═C3C4═NC(C(NC(C)C)═O)═CS4 |
| Example 1491 | C1═CC(═CC═C1F)C3═NOC(CNCC(═O)C2═CC═CC═C2)═C3C4═NC(C(NC(C)C)═O)═CS4 |
| Example 1492 | C1═CC(═CC═C1F)C2═NOC(CNCCCNC(OC(C)(C)C)═O)═C2C3═NC(C(NC(C)C)═O)═CS3 |
| Example 1493 | C1═CC(═CC═C1F)C3═NOC(CNCC2═CC═C(C═C2)C(F)(F)F)═C3C4═NC(C(NC(C)C)═O)═CS4 |
| Example 1494 | C1═CC(═CC═C1F)C3═NOC(CNCC2═CC═CC(═C2)C(F)(F)F)═C3C4═NC(C(NC(C)C)═O)═CS4 |
| Example 1495 | C1═CC(═CC═C1F)C3═NOC(CNCC2═CC═C(Cl)C(Cl)═C2)═C3C4═NC(C(NC(C)C)═O)═CS4 |
| Example 1496 | C1═CC(═CC═C1F)C3═NOC(CNCC2═CC═C(Cl)C═C2Cl)═C3C4═NC(C(NC(C)C)═O)═CS4 |
| Example 1497 | C1═CC(═CC═C1F)C4═NOC(CNC3CCN(CC2═CC═CC═C2)C3)═C4C5═NC(C(NC(C)C)═O)═CS5 |
| Example 1498 | C1═CC(═CC═C1F)C3═NOC(CNCCC═2C(═CC═C(OC)C═2)OC)═C3C4═NCC(C(NC(C)C)═O)═CS4 |
| Example 1499 | C1═CC(═CC═C1F)C3═NOC(CNCC2═CC═C(N(C)C)C═C2)═C3C4═NC(C(NC(C)C)═O)═CS4 |
| Example 1500 | C1═CC(═CC═C1F)C3═NOC(CNCC2═CC═C(OC)C(═C2)O)═C3C4═NC(C(NC(C)C)═O)═CS4 |
| Example 1501 | C1═CC(═CC═C1F)C4═NOC(CNC3CCN(CC2═CC═CC═C2)CC3)═C4C5═NC(C(NC(C)C)═O)═CS5 |
| Example 1502 | C1═CC(═CC═C1F)C3═NOC(CNCC2═CC═CC═C2OC(F)(F)F)═C3C4═NC(C(NC(C)C)═O)═CS4 |
| Example 1503 | C1═CC(═CC═C1F)C3═NOC(CNCCC2═CC═C(C═C2)S(═O)(═O)N)═C3C4═NC(C(NC(C)C)═O)═CS4 |
| Example 1504 | C1═CC(═CC═C1F)C3═NOC(CNCC(C2═CC═C(OC)C═C2)═O)═C3C4═NC(C(NC(C)C)═O)═CS4 |
| Example 1505 | C1═CC(═CC═C1F)C4═NOC(CNCCC3═CC═C2OCOC2═C3)═C4C5═NC(C(NC(C)C)═O)═CS5 |
| Example 1506 | C1═CC(═CC═C1F)C4═NOC(CNCCC(C2═CC═CC═C2)C3═CC═CC═C3)═C4C5═NC(C(NC(C)C)═O)═CS5 |
| Example 1507 | C1═CC(═CC═C1F)C3═NOC(CNCCNS(C2═CC═C(C)C═C2)(═O)═O)═C3C4═NC(C(NC(C)C)═O)═CS4 |

Examples Prepared with 2° Amines:

| | |
|---|---|
| Example 1508 | CN(CC═1ON═C(C═1C═2SC═C(N═2)C(N(C(C)C)[H])═O)C═3C═CC(═CC═3)F)C |
| Example 1509 | O4CCN(CC═1ON═C(C═1C═2SC═C(N═2)C(N(C(C)C)[H])═O)C═3C═CC(═CC═3)F)CC4 |
| Example 1510 | C1═CC(═CC═C1F)C2═NOC(CBr)═C2C3═NC(C(N([H])C(C)C)═O)═CS3 |
| Example 1511 | C1═CC(═CC═C1F)C2═NOC(CN(CC)C)═C2C3═NC(C(NC(C)C)═O)═CS3 |
| Example 1512 | C1═CC(═CC═C1F)C2═NOC(CN(C)CC#C)═C2C3═NC(C(NC(C)C)═O)═CS3 |
| Example 1513 | C1═CC(═CC═C1F)C2═NOC(CN(CC)CC)═C2C3═NC(C(NC(C)C)═O)═CS3 |
| Example 1514 | C1═CC(═CC═C1F)C2═NOC(CN(CCC)C)═C2C3═NC(C(NC(C)C)═O)═CS3 |
| Example 1515 | C1═CC(═CC═C1F)C2═NOC(CN(C)CCO)═C2C3═NC(C(NC(C)C)═O)═CS3 |
| Example 1516 | C1═CC(═CC═C1F)C2═NOC(CN(C)CCC#N)C2C3═NC(C(NC(C)C)═O)═CS3 |
| Example 1517 | C1═CC(═CC═C1F)C2═NOC(CN(C)CC(C)C)═C2C3═NC(C(NC(C)C)═O)═CS3 |
| Example 1518 | C1═CC(═CC═C1F)C2═NOC(CN(CC)C(C)C)═C2C3═NC(C(NC(C)C)═O)═CS3 |
| Example 1519 | C1═CC(═CC═C1F)C2═NOC(CN(CCCC)C)═C2C3═NC(C(NC(C)C)═O)═CS3 |
| Example 1520 | C1═CC(═CC═C1F)C2═NOC(CN(CC)CCO)═C2C3═NC(C(NC(C)C)═O)═CS3 |
| Example 1521 | C1═CC(═CC═C1F)C3═NOC(CN2CSCC2)═C3C4═NC(C(NC(C)C)═O)═CS4 |
| Example 1522 | C1═CC(═CC═C1F)C3═NOC(CN2CCCCC2C)═C3C4═NC(C(NC(C)C)═O)═CS4 |
| Example 1523 | C1═CC(═CC═C1F)C3═NOC(CN2CCC(C)CC2)═C3C4═NC(C(NC(C)C)═O)═CS4 |
| Example 1524 | C1═CC(═CC═C1F)C3═NOC(CN2CCN(C)CC2)═C3C4═NC(C(NC(C)C)═O)═CS4 |
| Example 1525 | C1═CC(═CC═C1F)C3═NOC(CN2CCCC2CO)═C3C4═NC(C(NC(C)C)═O)═CS4 |
| Example 1526 | C1═CC(═CC═C1F)C3═NOC(CN2CCC[C@@H]2CO)═C3C4═NC(C(NC(C)C)═O)═CS4 |
| Example 1527 | C1═CC(═CC═C1F)C2═NOC(CN(CCCC)CC)═C2C3═NC(C(NC(C)C)═O)═CS3 |
| Example 1528 | C1═CC(═CC═C1F)C2═NOC(CN(CCC)CCC)═C2C3═NC(C(NC(C)C)═O)═CS3 |
| Example 1529 | C1═CC(═CC═C1F)C3═NOC(CN2CCSCC2)═C3C4═NC(C(NC(C)C)═O)═CS4 |
| Example 1530 | C1═CC(═CC═C1F)C2═NOC(CN(CCO)CCO)═C2C3═NC(C(NC(C)C)═O)═CS3 |
| Example 1531 | C1═CC(═CC═C1F)C3═NOC(CN2CC(C)CC(C2)C)═C3C4═NC(C(NC(C)C)═O)═CS4 |
| Example 1532 | C1═CC(═CC═C1F)C3═NOC(CN(C)C2CCCCC2)═C3C4═NC(C(NC(C)C)═O)═CS4 |
| Example 1533 | C1═CC(═CC═C1F)C3═NOC(CN2C(CCCC2C)C)═C3C4═NC(C(NC(C)C)═O)═CS4 |
| Example 1534 | C1═CC(═CC═C1F)C3═NOC(CN2CCC(N(C)C)C2)═C3C4═NC(C(NC(C)C)═O)═CS4 |
| Example 1535 | C1═CC(═CC═C1F)C3═NOC(CN2C[C@@H](C)N[C@H](C2)C)═C3C4═NC(C(NC(C)C)═O)═CS4 |
| Example 1536 | C1═CC(═CC═C1F)C3═NOC(CN2CCCC(CO)C2)═C3C4═NC(C(NC(C)C)═O)═CS4 |
| Example 1537 | C1═CC(═CC═C1F)C3═NOC(CN2CCC[C@H]2COC)═C3C4═NC(C(NC(C)C)═O)═CS4 |
| Example 1538 | C1═CC(═CC═C1F)C3═NOC(CN2CC(C)OC(C2)C)═C3C4═NC(C(NC(C)C)═O)═CS4 |
| Example 1539 | C1═CC(═CC═C1F)C3═NOC(CN2CCCCC2CO)═C3C4═NC(C(NC(C)C)═O)═CS4 |
| Example 1540 | C1═CC(═CC═C1F)C2═NOC(CN(CC)CCCCO)═C2C3═NC(C(NC(C)C)═O)═CS3 |
| Example 1541 | C1═CC(═CC═C1F)C4═NOC(CN2CC3═CC═CC═C3C2)═C4C5═NC(C(NC(C)C)═O)═CS5 |
| Example 1542 | C1═CC(═CC═C1F)C3═NOC(CN(C)CC2═CC═CC═C2)═C3C4═NC(C(NC(C)C)═O)═CS4 |
| Example 1543 | C1═CC(═CC═C1F)C3═NOC(CN(CC)C2CCCCC2)═C3C4═NC(C(NC(C)C)═O)═CS4 |
| Example 1544 | C1═CC(═CC═C1F)C3═NOC(CN2CCC(C(N)═O)CC2)═C3C4═NC(C(NC(C)C)═O)═CS4 |
| Example 1545 | C1═CC(═CC═C1F)C3═NOC(CN2CC[C@H](NC(C)═O)C2)═C3C4═NC(C(NC(C)C)═O)═CS4 |
| Example 1546 | C1═CC(═CC═C1F)C3═NOC(CN2CC[C@H](NC(C)═O)C2)═C3C4═NC(C(NC(C)C)═O)═CS4 |
| Example 1547 | C1═CC(═CC═C1F)C3═NOC(CN2CCCC(C(N)═O)C2)═C3C4═NC(C(NC(C)C)═O)═CS4 |
| Example 1548 | C1═CC(═CC═C1F)C3═NOC(CN2CCCCC2CCO)═C3C4═NC(C(NC(C)C)═O)═CS4 |
| Example 1549 | C1═CC(═CC═C1F)C4═NOC(CN2CC3═CC═CC═C3CC2)═C4C5═NC(C(NC(C)C)═O)═CS5 |
| Example 1550 | C1═CC(═CC═C1F)C3═NOC(CN(CC)CC2═CC═CC═C2)═C3C4═NC(C(NC(C)C)═O)═CS4 |
| Example 1551 | C1═CC(═CC═C1F)C3═NOC(CN(CC)CC2═CC═CC═C2)═C3C4═NC(C(NC(C)C)═O)═CS4 |
| Example 1552 | C1═CC(═CC═C1F)C3═NOC(CN(C)CCC2═CC═CC═C2)═C3C4═NC(C(NC(C)C)═O)═CS4 |
| Example 1553 | C1═CC(═CC═C1F)C3═NOC(CN(C)CCC2═CC═CC═N2)═C3C4═NC(C(NC(C)C)═O)═CS4 |
| Example 1554 | C1═CC(═CC═C1F)C3═NOC(CN(C)CCC2═CC═CC═N2)═C3C4═NC(C(NC(C)C)═O)═CS4 |
| Example 1555 | C1═CC(═CC═C1F)C4═NOC(CN2C3CCCCC3CCC2)═C4C5═NC(C(NC(C)C)═O)═CS5 |
| Example 1556 | C1═CC(═CC═C1F)C3═NOC(CN(CC═C)C2CCCCC2)═C3C4═NC(C(NC(C)C)═O)═CS4 |
| Example 1557 | C1═CC(═CC═C1F)C3═NOC(CN2CCC(C(OC)═O)CC2)═C3C4═NC(C(NC(C)C)═O)═CS4 |
| Example 1558 | C1═CC(═CC═C1F)C3═NOC(CN(C(C)C)CC2═CC═CC═C2)═C3C4═NC(C(NC(C)C)═O)═CS4 |
| Example 1559 | C1═CC(═CC═C1F)C3═NOC(CN(CCO)CC2═CC═CC═C2)═C3C4═NC(C(NC(C)C)═O)═CS4 |
| Example 1560 | C1═CC(═CC═C1F)C3═NOC(CN(C)CC(O)C2═CC═CC═C2)═C3C4═NC(C(NC(C)C)═O)═CS4 |
| Example 1561 | C1═CC(═CC═C1F)C3═NOC(CN2CCC(O)(O)CC2)═C3C4═NC(C(NC(C)C)═O)═CS4 |
| Example 1562 | C1═CC(═CC═C1F)C3═NOC(CN2CCC(C(OCC)═O)CC2)═C3C4═NC(C(NC(C)C)═O)═CS4 |
| Example 1563 | C1═CC(═CC═C1F)C3═NOC(CN2CCCC(C(OCC)═O)C2)═C3C4═NC(C(NC(C)C)═O)═CS4 |
| Example 1564 | C1═CC(═CC═C1F)C3═NOC(CN2CCN(C(OCC)═O)CC2)═C3C4═NC(C(NC(C)C)═O)═CS4 |
| Example 1565 | C1═CC(═CC═C1F)C3═NOC(CN(CCC#N)CC2═CC═CC═C2)═C3C4═NC(C(NC(C)C)═O)═CS4 |
| Example 1566 | C1═CC(═CC═C1F)C3═NOC(CN(CCC#N)CC2═CC═CN═C2)═C3C4═NC(C(NC(C)C)═O)═CS4 |
| Example 1567 | C1═CC(═CC═C1F)C4═NOC(CN2CCN(CC2)C3═CC═CC═C3)═C4C5═NC(C(NC(C)C)═O)═CS5 |
| Example 1568 | C1═CC(═CC═C1F)C4═NOC(CN2CCN(CC2)C3═CC═CC═N3)═C4C5═NC(C(NC(C)C)═O)═CS5 |
| Example 1569 | C1═CC(═CC═C1F)C3═NOC(CN(CCCC)CC2═CC═CC═C2)═C3C4═NC(C(NC(C)C)═O)═CS4 |
| Example 1570 | C1═CC(═CC═C1F)C3═NOC(CN([C@H](C)C2═CC═CC═C2)CCO)═C3C4═NC(C(NC(C)C)═O)═CS4 |
| Example 1571 | C1═CC(═CC═C1F)C3═NOC(CN(CCCO)CC2═CC═CC═N2)═C3C4═NC(C(NC(C)C)═O)═CS4 |
| Example 1572 | C1═CC(═CC═C1F)C3═NOC(CN(C)CC(O)C2═CC═C(O)C═C2)═C3C4═NC(C(NC(C)C)═O)═CS4 |
| Example 1573 | C1═CC(═CC═C1F)C4═NOC(CN3CCN(C2CCCCC2)CC3)═C4C5═NC(C(NC(C)C)═O)═CS5 |
| Example 1574 | C1═CC(═CC═C1F)C4═NOC(CN3CCC(CC2═CC═CC═C2)CC3)═C4C5═NC(C(NC(C)C)═O)═CS5 |
| Example 1575 | C1═CC(═CC═C1F)C4═NOC(CN3CCN(CC2═CC═CC═C2)CC3)═C4C5═NC(C(NC(C)C)═O)═CS5 |
| Example 1576 | C1═CC(═CC═C1F)C4═NOC(CN3CCCN(CC2═CC═CC═C2)CC3)═C4C5═NC(C(NC(C)C)═O)═CS5 |
| Example 1577 | C1═CC(═CC═C1F)C4═NOC(CN3CCN(C2═CC═C(O)C═C2)CC3)═C4C5═NC(C(NC(C)C)═O)═CS5 |
| Example 1578 | C1═CC(═CC═C1F)C3═NOC(CN(CCN(C)C)CC2═CC═CC═C2)═C3C4═NC(C(NC(C)C)═O)═CS4 |
| Example 1579 | C1═CC(═CC═C1F)C4═NOC(CN3CCN(C2═CC═CC═C2F)CC3)═C4C5═NC(C(NC(C)C)═O)═CS5 |
| Example 1580 | C1═CC(═CC═C1F)C4═NOC(CN3CCN(C2═CC═C(F)C═C2)CC3)═C4C5═NCC(C(NC(C)C)═O)═CS5 |
| Example 1581 | C1═CC(═CC═C1F)C4═NOC(CN3CCN(CC2CCCCC2)CC3)═C4C5═NC(C(NC(C)C)═O)═CS5 |
| Example 1582 | C1═CC(═CC═C1F)C3═NOC(CN(C)C[C@H](O)C2═CC═C(O)C(O)═C2)═C3C4═NC(C(NC(C)C)═O)═CS4 |
| Example 1583 | C1═CC(═CC═C1F)C3═NOC(CN2C(CC(OCC)═O)C(NCC2)═O)═C3C4═NC(C(NC(C)C)═O)═CS4 |
| Example 1584 | C1═CC(═CC═C1F)C3═NOC(CN2CCN(C(OC(C)(C)C)═O)CC2)═C3C4═NC(C(NC(C)C)═O)═CS4 |
| Example 1585 | C1═CC(═CC═C1F)C3═NOC(CN2CCC(NC(OC(C)(C)C)═O)C2)═C3C4═NC(C(NC(C)C)═O)═CS4 |
| Example 1586 | C1═CC(═CC═C1F)C4═NOC(CN3CCN(C2═CC═CC═C2C#N)CC3)═C4C5═NC(C(NC(C)C)═O)═CS5 |
| Example 1587 | C1═CC(═CC═C1F)C4═NOC(CN3CCN(C2═CC═C(C#N)C═N2)CC3)═C4C5═NC(C(NC(C)C)═O)═CS5 |
| Example 1588 | C1═CC(═CC═C1F)C4═NOC(CN3CCN(C═2C═CC═C(C)C═2C)CC3)═C4C5═NC(C(NC(C)C)═O)═CS5 |
| Example 1589 | C1═CC(═CC═C1F)C4═NOC(CN3CCN(C═2C═CC═C(C)C═2C)CC3)═C4C5═NC(C(NC(C)C)═O)═CS5 |
| Example 1590 | C1═CC(═CC═C1F)C4═NOC(CN2CC(C)N(CC2)C3═CC═CC(C)═C3)═C4C5═NC(C(NC(C)C)═O)═CS5 |
| Example 1591 | C1═CC(═CC═C1F)C4═NOC(CN3CCN(CCC2═CC═CC═C2)CC3)═C4C5═NC(C(NC(C)C)═O)═CS5 |
| Example 1592 | C1═CC(═CC═C1F)C4═NOC(CN3CCN(C2═CC═C(C)C(═C2)C)CC3)═C4C5═NC(C(NC(C)C)═O)═CS5 |
| Example 1593 | C1═CC(═CC═C1F)C4═NOC(CN3CCN(C2═CC═C(C)C═C2C)CC3)═C4C5═NC(C(NC(C)C)═O)═CS5 |
| Example 1594 | C1═CC(═CC═C1F)C4═NOC(CN3CCN(C═2C(═CC═C(C)C═2)C)CC3)═C4C5═NC(C(NC(C)C)═O)═CS5 |
| Example 1595 | C1═CC(═CC═C1F)C4═NOC(CN3CCN(C2═CC═C(OC)C═C2)CC3)═C4C5═NC(C(NC(C)C)═O)═CS5 |
| Example 1596 | C1═CC(═CC═C1F)C4═NOC(CN3CCN(C2═CC═CC═C2OC)CC3)═C4C5═NC(C(NC(C)C)═O)═CS5 |
| Example 1597 | C1═CC(═CC═C1F)C4═NOC(CN3CCN(C═2C═CC═C(OC)C═2)CC3)═C4C5═NC(C(NC(C)C)═O)═CS5 |
| Example 1598 | C1═CC(═CC═C1F)C3═NOC(CN(C)CCC2═CC═C(OC)C(OC)═C2)═C3C4═NC(C(NC(C)C)═O)═CS4 |
| Example 1599 | C1═CC(═CC═C1F)C4═NOC(CN3CCN(C═2C═CC═C(Cl)C═2)CC3)═C4C5═NC(C(NC(C)C)═O)═CS5 |
| Example 1600 | C1═CC(═CC═C1F)C4═NOC(CN3CCN(C2═CC═C(Cl)C═C2)CC3)═C4C5═NC(C(NC(C)C)═O)═CS5 |
| Example 1601 | C1═CC(═CC═C1F)C4═NOC(CN3CCN(C2═CC═C(Cl)C═C2)CC3)═C4C5═NC(C(NC(C)C)═O)═CS5 |
| Example 1602 | C1═CC(═CC═C1F)C4═NOC(CN3CCN(C2═CC═C(F)C═C2F)CC3)═C4C5═NC(C(NC(C)C)═O)═CS5 |
| Example 1603 | C1═CC(═CC═C1F)C4═NOC(CN(CC2═CC═CN═C2)CC3═CC═CN═C3)═C4C5═NC(C(NC(C)C)═O)═CS5 |
| Example 1604 | C1═CC(═CC═C1F)C4═NOC(CN3CCN(CCN2CCOCC2)CC3)═C4C5═NC(C(NC(C)C)═O)═CS5 |
| Example 1605 | C1═CC(═CC═C1F)C3═NOC(CN2CCC(N(C)C(═O)OC(C)(C)C)C2)═C3C4═NC(C(NC(C)C)═O)═CS4 |
| Example 1606 | C1═CC(═CC═C1F)C4═NOC(CN3CCN(C2═CC═C(C(C)═O)C═C2)CC3)═C4C5═NC(C(NC(C)C)═O)═CS5 |
| Example 1607 | C1═CC(═CC═C1F)C3═NOC(CN(CCN(CC)CC)CC2═CC═CC═C2)═C3C4═NC(C(NC(C)C)═O)═CS4 |
| Example 1608 | C1═CC(═CC═C1F)C4═NOC(CN3CCN(C2═CC═C([N+]([O−])═O)C═C2)CC3)═C4C5═NC(C(NC(C)C)═O)═CS5 |
| Example 1609 | C1═CC(═CC═C1F)C4═NOC(CN(C)CC2═CC═CC3═CC═CC═C23)═C4C5═NC(C(NC(C)C)═O)═CS5 |
| Example 1610 | C1═CC(═CC═C1F)C4═NOC(CN3CCN(C2═CC(═CC═C2C)Cl)CC3)═C4C5═NC(C(NC(C)C)═O)═CS5 |
| Example 1611 | C1═CC(═CC═C1F)C3═NOC(CN(C)CC2═CC(OC)═C(OC)C(OC)═C2)═C3C4═NC(C(NC(C)C)═O)═CS4 |
| Example 1612 | C1═CC(═CC═C1F)C4═NOC(CN(CCC2═CC═CC═C2)CC3═CC═CC═C3)═C4C5═NC(C(NC(C)C)═O)═CS5 |
| Example 1613 | C1═CC(═CC═C1F)C4═NOC(CN(CCC2═CC═CC═C2)CC3═CC═CC═C3)═C4C5═NC(C(NC(C)C)═O)═CS5 |
| Example 1614 | C1═CC(═CC═C1F)C4═NOC(CN(C)C(CC2═CC═CC═C2)C3═CC═CC═C3)═C4C5═NC(C(NC(C)C)═O)═CS5 |
| Example 1615 | C1═CC(═CC═C1F)C4═NOC(CN2CCC(O)(CC2)C3═CC═C(Cl)C═C3)═C4C5═NC(C(NC(C)C)═O)═CS5 |
| Example 1616 | C1═CC(═CC═C1F)C3═NOC(CN(CC2═CC═C(Cl)C═C2Cl)CC#C)═C3C4═NC(C(NC(C)C)═O)═CS4 |
| Example 1617 | C1═CC(═CC═C1F)C4═NOC(CN(CC)C(CC2═CC═CC═C2)C═3OC═CC═3)═C4C5═NC(C(NC(C)C)═O)═CS5 |
| Example 1618 | C1═CC(═CC═C1F)C3═NOC(CN2CCN(C(OC(C)(C)C)═O)C[C@H]2CO)═C3C4═NC(C(NC(C)C)═O)═CS4 |
| Example 1619 | C1═CC(═CC═C1F)C4═NOC(CN3CCN(C2═CC═C(C(C)(C)C)C═C2)CC3)═C4C5═NC(C(NC(C)C)═O)═CS5 |
| Example 1620 | C1═CC(═CC═C1F)C5═NOC(CN4CCN(CC3═CC═C2OCOC2═C3)CC4)═C5C6═NC(C(NC(C)C)═O)═CS6 |
| Example 1621 | C1═CC(═CC═C1F)C4═NOC(CN2CCC═3C═C(OC)C(═CC═3C2)OC)═C4C5═NC(C(NC(C)C)═O)═CS5 |
| Example 1622 | C1═CC(═CC═C1F)C4═NOC(CN3CCN(C2═CC═C(C(F)(F)F)C═C2)CC3)═C4C5═NC(C(NC(C)C)═O)═CS5 |
| Example 1623 | C1═CC(═CC═C1F)C4═NOC(CN3CCN(C═2C═CC═C(C(F)(F)F)C═2)CC3)═C4C5═NC(C(NC(C)C)═O)═CS5 |
| Example 1624 | C1═CC(═CC═C1F)C4═NOC(CN3CCN(C2═CC═C(Cl)C(═C2)Cl)CC3)═C4C5═NC(C(NC(C)C)═O)═CS5 |
| Example 1625 | C1═CC(═CC═C1F)C4═NOC(CN3CCN(C2═CC═C(Cl)C(═C2)Cl)CC3)═C4C5═NC(C(NC(C)C)═O)═CS5 |
| Example 1626 | C1═CC(═CC═C1F)C4═NOC(CN3CCN(C2═CC═C(C(F)(F)F)C═N2)CC3)═C4C5═NC(C(NC(C)C)═O)═CS5 |
| Example 1627 | C1═CC(═CC═C1F)C4═NOC(CN3CCN(C2═CC═CC═C2Cl)CC3)═C4C5═NC(C(NC(C)C)═O)═CS5 |
| Example 1628 | C1═CC(═CC═C1F)C4═NOC(CN3CCN(C2═CC═C(OC)C(═C2)OC)CC3)═C4C5═NC(C(NC(C)C)═O)═CS5 |
| Example 1629 | C1═CC(═CC═C1F)C4═NOC(CN3CCCC(C2═CC═C(C(F)(F)F)C═C2)C3)═C4C5═NC(C(NC(C)C)═O)═CS5 |

Examples Prepared with Anilines:

| | |
|---|---|
| Example 1630 | C1═CC(═CC═C1F)C3═NOC(CNC2═CC═CC═C2)═C3C4═NC(C(NC(C)C)═O)═CS4 |
| Example 1631 | C1═CC(═CC═C1F)C3═NOC(CNC═2C═CC═C(C)C═2)═C3C4═NC(C(NC(C)C)═O)═CS4 |
| Example 1632 | C1═CC(═CC═C1F)C3═NOC(CNC2═CC═C(C)C═C2)═C3C4═NC(C(NC(C)C)═O)═CS4 |
| Example 1633 | C1═CC(═CC═C1F)C3═NOC(CNC2═CC═CC═C2C)═C3C4═NC(C(NC(C)C)═O)═CS4 |
| Example 1634 | C1═CC(═CC═C1F)C3═NOC(CNC2═CC═C(C)C═N2)═C3C4═NC(C(NC(C)C)═O)═CS4 |
| Example 1635 | C1═CC(═CC═C1F)C3═NOC(CNC═2C═CC═C(C)N═2)═C3C4═NC(C(NC(C)C)═O)═CS4 |
| Example 1636 | C1═CC(═CC═C1F)C3═NOC(CNC═2N═CC═C(C)C═2)═C3C4═NC(C(NC(C)C)═O)═CS4 |
| Example 1637 | C1═CC(═CC═C1F)C3═NOC(CNC2═NC═CC═C2C)═C3C4═NC(C(NC(C)C)═O)═CS4 |
| Example 1638 | C1═CC(═CC═C1F)C3═NOC(CNC2═CC═CC═C2F)═C3C4═NC(C(NC(C)C)═O)═CS4 |
| Example 1639 | C1═CC(═CC═C1F)C3═NOC(CNC2═CC═C(F)C═C2)═C3C4═NC(C(NC(C)C)═O)═CS4 |
| Example 1640 | C1═CC(═CC═C1F)C3═NOC(CNC═2C(═CC═C(C)C═2)C)═C3C4═NC(C(NC(C)C)═O)═CS4 |
| Example 1641 | C1═CC(═CC═C1F)C3═NOC(CNC2═CC═C(C)C(═C2)C)═C3C4═NC(C(NC(C)C)═O)═CS4 |
| Example 1642 | C1═CC(═CC═C1F)C3═NOC(CNC2═CC═CC═C2CC)═C3C4═NC(C(NC(C)C)═O)═CS4 |
| Example 1643 | C1═CC(═CC═C1F)C3═NOC(CNC2═CC═CC═C2CC)═C3C4═NC(C(NC(C)C)═O)═CS4 |
| Example 1644 | C1═CC(═CC═C1F)C3═NOC(CNC═2C═CC═C(C)C═2C)═C3C4═NC(C(NC(C)C)═O)═CS4 |
| Example 1645 | C1═CC(═CC═C1F)C3═NOC(CNC2═CC═C(C)C═C2C)═C3C4═NC(C(NC(C)C)═O)═CS4 |
| Example 1646 | C1═CC(═CC═C1F)C3═NOC(CNC═2C═C(C═C(C)C═2)C)═C3C4═NC(C(NC(C)C)═O)═CS4 |
| Example 1647 | C1═CC(═CC═C1F)C3═NOC(CNC═2C═CC═C(OC)C═2)═C3C4═NC(C(NC(C)C)═O)═CS4 |
| Example 1648 | C1═CC(═CC═C1F)C3═NOC(CNC2═CC═C(OC)C═C2)═C3C4═NC(C(NC(C)C)═O)═CS4 |
| Example 1649 | C1═CC(═CC═C1F)C3═NOC(CNC2═CC═CC═C2OC)═C3C4═NC(C(NC(C)C)═O)═CS4 |
| Example 1650 | C1═CC(═CC═C1F)C3═NOC(CNC2═CN═CC═C2OC)═C3C4═NC(C(NC(C)C)═O)═CS4 |
| Example 1651 | C1═CC(═CC═C1F)C3═NOC(CNC2═CC═C(C)C(═C2)F)═C3C4═NC(C(NC(C)C)═O)═CS4 |
| Example 1652 | C1═CC(═CC═C1F)C3═NOC(CNC2═C(C)C(═CC═C2)F)═C3C4═NC(C(NC(C)C)═O)═CS4 |
| Example 1653 | C1═CC(═CC═C1F)C3═NOC(CNC2═CC(F)═CC(F)═C2)═C3C4═NC(C(NC(C)C)═O)═CS4 |
| Example 1654 | C1═CC(═CC═C1F)C3═NOC(CNC2═CC═C(F)C═C2F)═C3C4═NC(C(NC(C)C)═O)═CS4 |
| Example 1655 | C1═CC(═CC═C1F)C3═NOC(CNC2═CC═C(F)C═C2F)═C3C4═NC(C(NC(C)C)═O)═CS4 |
| Example 1656 | C1═CC(═CC═C1F)C4═NOC(CNC3═CC═C2CCCC2═C3)═C4C5═NC(C(NC(C)C)═O)═CS5 |
| Example 1657 | C1═CC(═CC═C1F)C3═NOC(CNC2═CC═CC═C2C(C)C)═C3C4═NC(C(NC(C)C)═O)═CS4 |
| Example 1658 | C1═CC(═CC═C1F)C3═NOC(CNC═2C═CC═C(C(C)═O)C═2)═C3C4═NC(C(NC(C)C)═O)═CS4 |
| Example 1659 | C1═CC(═CC═C1F)C3═NOC(CNC2═CC═C(C(C)═O)C═C2)═C3C4═NC(C(NC(C)C)═O)═CS4 |
| Example 1660 | C1═CC(═CC═C1F)C3═NOC(CNC2═C(C═CC═C2CC)C)═C3C4═NC(C(NC(C)C)═O)═CS4 |
| Example 1661 | C1═CC(═CC═C1F)C3═NOC(CNC2═C(C═C(C)C═C2C)C)═C3C4═NC(C(NC(C)C)═O)═CS4 |
| Example 1662 | C1═CC(═CC═C1F)C4═NOC(CNC3═CC═C2OCOC2═C3)═C4C5═NC(C(NC(C)C)═O)═CS5 |
| Example 1663 | C1═CC(═CC═C1F)C3═NOC(CNC2═CC═C(OC)C═C2C)═C3C4═NC(C(NC(C)C)═O)═CS4 |
| Example 1664 | C1═CC(═CC═C1F)C3═NOC(CNC═2C═CC═C(OCC)C═2)═C3C4═NC(C(NC(C)C)═O)═CS4 |
| Example 1665 | C1═CC(═CC═C1F)C3═NOC(CNC2═CC═CC═C2OCC)═C3C4═NC(C(NC(C)C)═O)═CS4 |
| Example 1666 | C1═CC(═CC═C1F)C3═NOC(CNC2═CC═CC═C2SC)═C3C4═NC(C(NC(C)C)═O)═CS4 |
| Example 1667 | C1═CC(═CC═C1F)C3═NOC(CNC2═C(C)C(═CC═C2)Cl)═C3C4═NC(C(NC(C)C)═O)═CS4 |
| Example 1668 | C1═CC(═CC═C1F)C3═NOC(CNC2═CC═C(C)C(═C2)Cl)═C3C4═NC(C(NC(C)C)═O)═CS4 |
| Example 1669 | C1═CC(═CC═C1F)C4═NOC(CNC3═CN═C2C═CC═CC2═C3)═C4C5═NC(C(NC(C)C)═O)═CS5 |
| Example 1670 | C1═CC(═CC═C1F)C3═NOC(CNC2═CC(F)═C(F)C═C2F)═C3C4═NC(C(NC(C)C)═O)═CS4 |
| Example 1671 | C1═CC(═CC═C1F)C4═NOC(CNC2CCCC3═CC═CC═C23)═C4C5═NC(C(NC(C)C)═O)═CS5 |
| Example 1672 | C1═CC(═CC═C1F)C3═NOC(CNC2═CC═CC═C2C(C)(C)C)═C3C4═NC(C(NC(C)C)═O)═CS4 |
| Example 1673 | C1═CC(═CC═C1F)C3═NOC(CNC2═C(C═CC═C2C(C)C)C)═C3C4═NC(C(NC(C)C)═O)═CS4 |
| Example 1674 | C1═CC(═CC═C1F)C4═NOC(CNC3═CC═C2N═CSC2═C3)═C4C5═NC(C(NC(C)C)═O)═CS5 |
| Example 1675 | C1═CC(═CC═C1F)C3═NOC(CNC2═CC═C(NC(C)═O)C═C2)═C3C4═NC(C(NC(C)C)═O)═CS4 |
| Example 1676 | C1═CC(═CC═C1F)C3═NOC(CNC2═CC═C(NC(C)═O)N═C2)═C3C4═NC(C(NC(C)C)═O)═CS4 |
| Example 1677 | C1═CC(═CC═C1F)C4═NOC(CNC3═CC═C2OCCOC2═C3)═C4C5═NC(C(NC(C)C)═O)═CS5 |
| Example 1678 | C1═CC(═CC═C1F)C3═NOC(CNC2═CC═C(OC)C(═C2)OC)═C3C4═NC(C(NC(C)C)═O)═CS4 |
| Example 1679 | C1═CC(═CC═C1F)C4═NOC(CNC2═CC═CC═C2N3C═CC═C3)═C4C5═NC(C(NC(C)C)═O)═CS5 |
| Example 1680 | C1═CC(═CC═C1F)C3═NOC(CNC2═CC═C(C═C2)C(F)(F)F)═C3C4═NC(C(NC(C)C)═O)═CS4 |
| Example 1681 | C1═CC(═CC═C1F)C3═NOC(CNC2═CC═CC(═C2)C(F)(F)F)═C3C4═NC(C(NC(C)C)═O)═CS4 |
| Example 1682 | C1═CC(═CC═C1F)C3═NOC(CNC2═CC═C(Cl)C(Cl)═C2)═C3C4═NC(C(NC(C)C)═O)═CS4 |
| Example 1683 | C1═CC(═CC═C1F)C3═NOC(CNC2═C(C═CC═C2C)C(C)(C)C)═C3C4═NC(C(NC(C)C)═O)═CS4 |
| Example 1684 | C1═CC(═CC═C1F)C3═NOC(CNC2═CC═C(N(C)C)C═C2)═C3C4═NC(C(NC(C)C)═O)═CS4 |
| Example 1685 | C1═CC(═CC═C1F)C4═NOC(CNC2═CC═CC═C2C3═CC═CC═C3)═C4C5═NC(C(NC(C)C)═O)═CS5 |
| Example 1686 | C1═CC(═CC═C1F)C3═NOC(CNC2═CC═C(OC(F)(F)F)C═C2)═C3C4═NC(C(NC(C)C)═O)═CS4 |
| Example 1687 | C1═CC(═CC═C1F)C3═NOC(CNC2═C(C═CC═C2C(C)C)C(C)C)═C3C4═NC(C(NC(C)C)═O)═CS4 |
| Example 1688 | C1═CC(═CC═C1F)C3═NOC(CNC2═CC═C(F)C═C2C(F)(F)F)═C3C4═NC(C(NC(C)C)═O)═CS4 |
| Example 1689 | C1═CC(═CC═C1F)C3═NOC(CNC2═CC(═CC═C2F)C(F)(F)F)═C3C4═NC(C(NC(C)C)═O)═CS4 |
| Example 1690 | C1═CC(═CC═C1F)C3═NOC(CNC2═CC═C(F)C(═C2)C(F)(F)F)═C3C4═NC(C(NC(C)C)═O)═CS4 |
| Example 1691 | C1═CC(═CC═C1F)C3═NOC(CNC═2C(═CC═C(OCC)C═2)OCC)═C3C4═NC(C(NC(C)C)═O)═CS4 |
| Example 1692 | C1═CC(═CC═C1F)C3═NOC(CNC2═CC(Cl)═C(Cl)C(Cl)═C2)═C3C4═NC(C(NC(C)C)═O)═CS4 |
| Example 1693 | C1═CC(═CC═C1F)C4═NOC(CNC2═CC═CC═C2C(═O)C3═CC═CC═C3)═C4C5═NC(C(NC(C)C)═O)═CS5 |
| Example 1694 | C1═CC(═CC═C1F)C4═NOC(CNC2═CC═CC═C2COC3═CC═CC═C3)═C4C5═NC(C(NC(C)C)═O)═CS5 |
| Example 1695 | C1═CC(═CC═C1F)C4═NOC(CNC2═CC(═CC═C2OC)C3═CC═CC═C3)═C4C5═NC(C(NC(C)C)═O)═CS5 |
| Example 1696 | C1═CC(═CC═C1F)C4═NOC(CNC2═NC═CC═C2OCC3═CC═CC═C3)═C4C5═NC(C(NC(C)C)═O)═CS5 |
| Example 1697 | C1═CC(═CC═C1F)C3═NOC(CNC2═CC(═CC═C2C(F)(F)F)C(F)(F)F)═C3C4═NC(C(NC(C)C)═O)═CS4 |
| Example 1698 | C1═CC(═CC═C1F)C3═NOC(CNC2═CC(═CC(═C2)C(F)(F)F)C(F)(F)F)═C3C4═NC(C(NC(C)C)═O)═CS4 |
| Example 1699 | C1═CC(═CC═C1F)C3═NOC(CNC2═CC═C(C═C2C(F)(F)F)C(F)(F)F)═C3C4═NC(C(NC(C)C)═O)═CS4 |
| Example 1700 | C1═CC(═CC═C1F)C2═NOC(CBr)═C2C3═NC(C(N([H])C(C)C)═O)═CS3 |

### EXAMPLES 1701-2025

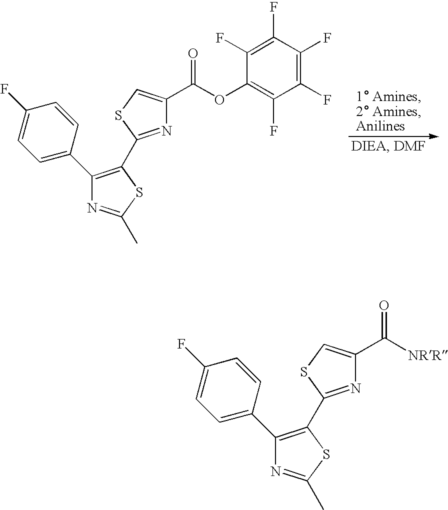 Starting pentafluorophenyl ester was prepared as described in Step 2 of Example 47. Where, 1° amines, 2° amines, and anilines were selected to afford Examples 1698-2025, which were prepared by General Procedure 2.

Examples Prepared with 1° Amines:

| | |
|---|---|
| Example 1701 | N3═C(SC═C3C(═O)NC1CCCC2═CC═CC═C12)C═4SC(C)═NC═4C5═CC═C(F)C═C5 |
| Example 1702 | N2═C(SC═C2C(═O)NC1═CC═C(OC)C(═C1)OC)C═3SC(C)═NC═3C4═CC═C(F)C═C4 |
| Example 1703 | N1═C(SC═C1C(═O)O)C═2SC(C)═NC═2C3═CC═C(F)C═C3 |
| Example 1704 | N2═C(SC═C2C(═O)N(CCC#N)CC1═CC═CC═C1)C═3SC(C)═NC═3C4═CC═C(F)C═C4 |
| Example 1705 | N1═C(SC═C1C(═O)NC)C═2SC(C)═NC═2C3═CC═C(F)C═C3 |
| Example 1706 | N1═C(SC═C1C(═O)NCC)C═2SC(C)═NC═2C3═CC═C(F)C═C3 |
| Example 1707 | N1═C(SC═C1C(═O)NCC#C)C═2SC(C)═NC═2C3═CC═C(F)C═C3 |
| Example 1708 | N1═C(SC═C1C(═O)NCC#N)C═2SC(C)═NC═2C3═CC═C(F)C═C3 |
| Example 1709 | N2═C(SC═C2C(═O)NC1CC1)C═3SC(C)═NC═3C4═CC═C(F)C═C4 |
| Example 1710 | N1═C(SC═C1C(═O)NCCC)C═2SC(C)═NC═2C3═CC═C(F)C═C3 |
| Example 1711 | N1═C(SC═C1C(═O)NCCN)C═2SC(C)═NC═2C3═CC═C(F)C═C3 |
| Example 1712 | N1═C(SC═C1C(═O)NCCO)C═2SC(C)═NC═2C3═CC═C(F)C═C3 |
| Example 1713 | N1═C(SC═C1C(═O)NCCC#N)C═2SC(C)═NC═2C3═CC═C(F)C═C3 |
| Example 1714 | N2═C(SC═C2C(═O)NC1CCC1)C═3SC(C)═NC═3C4═CC═C(F)C═C4 |
| Example 1715 | N2═C(SC═C2C(═O)NCC1CC1)C═3SC(C)═NC═3C4═CC═C(F)C═C4 |
| Example 1716 | N1═C(SC═C1C(═O)NC(CC)C)C═2SC(C)═NC═2C3═CC═C(F)C═C3 |
| Example 1717 | N1═C(SC═C1C(═O)NCC(C)C)C═2SC(C)═NC═2C3═CC═C(F)C═C3 |
| Example 1718 | N1═C(SC═C1C(═O)NCCCO)C═2SC(C)═NC═2C3═CC═C(F)C═C3 |
| Example 1719 | N1═C(SC═C1C(═O)NC(C)CO)C═2SC(C)═NC═2C3═CC═C(F)C═C3 |
| Example 1720 | N1═C(SC═C1C(═O)NCCOC)C═2SC(C)═NC═2C3═CC═C(F)C═C3 |
| Example 1721 | N2═C(SC═C2C(═O)NC1CCCC1)C═3SC(C)═NC═3C4═CC═C(F)C═C4 |
| Example 1722 | N1═C(SC═C1C(═O(NC(CCC)C)C═2SC(C)═NC═2C3═CC═C(F)C═C3 |
| Example 1723 | N1═C(SC═C1C(═O)NCC(CC)C)C═2SC(C)═NC═2C3═CC═C(F)C═C3 |
| Example 1724 | N1═C(SC═C1C(═O)NC(C(C)C)C)C═2SC(C)═NC═2C3═CC═C(F)C═C3 |
| Example 1725 | N1═C(SC═C1C(═O)NCCC(C)C)C═2SC(C)═NC═2C3═CC═C(F)C═C3 |
| Example 1726 | N1═C(SC═C1C(═O)NCC N(C)C)C═2SC(C)═NC═2C3═CC═C(F)C═C3 |
| Example 1727 | N1═C(SC═C1C(═O)NC(COC)C)C═2SC(C)═NC═2C3═CC═C(F)C═C3 |
| Example 1728 | N1═C(SC═C1C(═O)NC(CC)CO)C═2SC(C)═NC═2C3═CC═C(F)C═C3 |
| Example 1729 | N1═C(SC═C1C(═O)NC(CC)CO)C═2SC(C)═NC═2C3═CC═C(F)C═C3 |
| Example 1730 | N1═C(SC═C1C(═O)NCCCCO)C═2SC(C)═NC═2C3═CC═C(F)C═C3 |
| Example 1731 | N2═C(SC═C2C(═O)NCC═1OC═CC═1)C═3SC(C)═NC═3C4═CC═C(F)C═C4 |
| Example 1732 | N2═C(SC═C2C(═O)NC1CCCCC1)C═3SC(C)═NC═3C4═CC═C(F)C═C4 |
| Example 1733 | N2═C(SC═C2C(═O)NCC1CCCO1)C═3SC(C)═NC═3C4═CC═C(F)C═C4 |
| Example 1734 | N1═C(SC═C1C(═O)NCCC(C)(C)C)C═2SC(C)═NC═2C3═CC═C(F)C═C3 |
| Example 1735 | N1═C(SC═C1C(═O)NC(CC(C)C)C)C═2SC(C)═NC═2C3═CC═C(F)C═C3 |
| Example 1736 | N2═C(SC═C2C(═O)NC1CONC1═O)C═3SC(C)═NC═3C4═CC═C(F)C═C4 |
| Example 1737 | N2═C(SC═C2C(═O)NN1CCOCC1)C═3SC(C)═NC═3C4═CC═C(F)C═C4 |
| Example 1738 | N1═C(SC═C1C(═O)NC(C(C)C)CO)C═2SC(C)═NC═2C3═CC═C(F)C═C3 |
| Example 1739 | N2═C(SC═C2C(═O)NCC1═CC═CC═C1)C═3SC(C)═NC═3C4═CC═C(F)C═C4 |
| Example 1740 | N2═C(SC═C2C(═O)NCC1═CC═CN═C1)C═3SC(C)═NC═3C4═CC═C(F)C═C4 |
| Example 1741 | N2═C(SC═C2C(═O)NCC1═CC═CC═N1)C═3SC(C)═NC═3C4═CC═C(F)C═C4 |
| Example 1742 | N2═C(SC═C2C(═O)NCC1═CC═NC═C1)C═3SC(C)═NC═3C4═CC═C(F)C═C4 |
| Example 1743 | N2═C(SC═C2C(═O)NCC1═CC═CS1)C═3SC(C)═NC═3C4═CC═C(F)C═C4 |
| Example 1744 | N2═C(SC═C2C(═O)NC1CCC(C)CC1)C═3SC(C)═NC═3C4═CC═C(F)C═C4 |
| Example 1745 | N2═C(SC═C2C(═O)NCC1CCCCC1)C═3SC(C)═NC═3C4═CC═C(F)C═C4 |
| Example 1746 | N2═C(SC═C2C(═O)NC1CCCCCC1)C═3SC(C)═NC═3C4═CC═C(F)C═C4 |
| Example 1747 | N2═C(SC═C2C(═O)NC1CCCCC1C)C═3SC(C)═NC═3C4═CC═C(F)C═C4 |
| Example 1748 | N2═C(SC═C2C(═O)NCCN1CCCC1)C═3SC(C)═NC═3C4═CC═C(F)C═C4 |
| Example 1749 | N2═C(SC═C2C(═O)NN1 CCN(C)CC1)C═3SC(C)═NC═3C4═CC═C(F)C═C4 |
| Example 1750 | N1═C(SC═C1C(═O)NCC N(CC)CC)C═2SC(C)═NC═2C3═CC═C(F)C═C3 |
| Example 1751 | N2═C(SC═C2C(═O)NCC═1C═CC═C(C)C═1)C═3SC(C)═NC═3C4═CC═C(F)C═C4 |
| Example 1752 | N2═C(SC═C2C(═O)N[C@@H](C)C1═CC═CC═C1)C═3SC(C)═NC═3C4═CC═C(F)C═C4 |
| Example 1753 | N2═C(SC═C2C(═O)N[C@H](C)C1═CC═CC═C1)C═3SC(C)═NC═3C4═CC═C(F)C═C4 |
| Example 1754 | N2═C(SC═C2C(═O)NCCC1═CC═CC═C1)C═3SC(C)═NC═3C4═CC═C(F)C═C4 |
| Example 1755 | N2═C(SC═C2C(═O)NCC1═CC═CC═C1C)C═3SC(C)═NC═3C4═CC═C(F)C═C4 |
| Example 1756 | N2═C(SC═C2C(═O)NCC1═CC═C(C)C═C1)C═3SC(C)═NC═3C4═CC═C(F)C═C4 |
| Example 1757 | N2═C(SC═C2C(═O)NCC1═NC═C(C)N═C1)C═3SC(C)═NC═3C4═CC═C(F)C═C4 |
| Example 1758 | N2═C(SC═C2C(═O)NCC1═CC═CC(F)═C1)C═3SC(C)═NC═3C4═CC═C(F)C═C4 |
| Example 1759 | N2═C(SC═C2C(═O)NCC1═CC═C(F)C═C1)C═3SC(C)═NC═3C4═CC═C(F)C═C4 |
| Example 1760 | N2═C(SC═C2C(═O)NCC1═CC═CC═C1F)C═3SC(C)═NC═3C4═CC═C(F)C═C4 |
| Example 1761 | N2═C(SC═C2C(═O)NCCCN1C═CN═C1)C═3SC(C)═NC═3C4═CC═C(F)C═C4 |
| Example 1762 | N2═C(SC═C2C(═O)NCCC1CCCN1C)C═3SC(C)═NC═3C4═CC═C(F)C═C4 |
| Example 1763 | N2═C(SC═C2C(═O)NCCN1CCCCC1)C═3SC(C)═NC═3C4═CC═C(F)C═C4 |
| Example 1764 | N1═C(SC═C1C(═O)NC(CCCC(C)C)C)C═2SC(C)═NC═2C3═CC═C(F)C═C3 |
| Example 1765 | N2═C(SC═C2C(═O)NCCN1CCOCC1)C═3SC(C)═NC═3C4═CC═C(F)C═C4 |
| Example 1766 | N1═C(SC═C1C(═O)NC(CC(OCC)═O)C)C═2SC(C)═NC═2C3═CC═C(F)C═C3 |
| Example 1767 | N3═C(SC═C3C(═O)NC1CCC2═CC═CC═C12)C═4SC(C)═NC═4C5═CC═C(F)C═C5 |
| Example 1768 | N2═C(SC═C2C(═O)NCCC1═CC═C(C)C═C1)C═3SC(C)═NC═3C4═CC═C(F)C═C4 |
| Example 1769 | N2═C(SC═C2C(═O)NCC═1C(═CC═C(C)C═1)C)C═3SC(C)═NC═3C4═CC═C(F)C═C4 |
| Example 1770 | N2═C(SC═C2C(═O)N[C @@H](C)C1═CC═C(C)C═C1)C═3SC(C)═NC═3C4═CC═C(F)C═C4 |
| Example 1771 | N2═C(SC═C2C(═O)N[C@H](C)C1═CC═C(C)C═C1)C═3SC(C)═NC═3C4═CC═C(F)C═C4 |
| Example 1772 | N2═C(SC═C2C(═O)NCC(C)C1═CC═CC═C1)C═3SC(C)═NC═3C4═CC═C(F)C═C4 |
| Example 1773 | N2═C(SC═C2C(═O)NCC(C)C1═CC═CC═C1)C═3SC(C)═NC═3C4═CC═C(F)C═C4 |
| Example 1774 | N2═C(SC═C2C(═O)NCCCC1═CC═CC═C1)C═3SC(C)═NC═3C4═CC═C(F)C═C4 |
| Example 1775 | N2═C(SC═C2C(═O)NCC1═CC═C(C)C(═C1)C)C═3SC(C)═NC═3C4═CC═C(F)C═C4 |
| Example 1776 | N2═C(SC═C2C(═O)NC(C)CC1═CC═NC═C1)C═3SC(C)═NC═3C4═CC═C(F)C═C4 |
| Example 1777 | N2═C(SC═C2C(═O)NCCC1═CC═C(O)C═C1)C═3SC(C)═NC═3C4═CC═C(F)C═C4 |
| Example 1778 | N2═C(SC═C2C(═O)NCCOC1═CC═CC═C1)C═3SC(C)═NC═3C4═CC═C(F)C═C4 |
| Example 1779 | N2═C(SC═C2C(═O)NCC1═CC═CC═C1OC)C═3SC(C)═NC═3C4═CC═C(F)C═C4 |
| Example 1780 | N2═C(SC═C2C(═O)NCC1═CC═C(OC)C═C1)C═3SC(C)═NC═3C4═CC═C(F)C═C4 |
| Example 1781 | N2═C(SC═C2C(═O)NCC═1C═CC═C(OC)C═1)C═3SC(C)═NC═3C4═CC═C(F)C═C4 |
| Example 1782 | N2═C(SC═C2C(═O)NCCC1═CC═C(F)C═C1)C═3SC(C)═NC═3C4═CC═C(F)C═C4 |
| Example 1783 | N2═C(SC═C2C(═O)NCCC1═CC═CC═C1F)C═3SC(C)═NC═3C4═CC═C(F)C═C4 |
| Example 1784 | N2═C(SC═C2C(═O)NCCC1═CC═CC(F)═C1)C═3SC(C)═NC═3C4═CC═C(F)C═C4 |
| Example 1785 | N2═C(SC═C2C(═O)NCC1═CC═CC(Cl)═C1)C═3SC(C)═NC═3C4═CC═C(F)C═C4 |
| Example 1786 | N2═C(SC═C2C(═O)NCC1═CC═CC═C1Cl)C═3SC(C)═NC═3C4═CC═C(F)C═C4 |
| Example 1787 | N2═C(SC═C2C(═O)NCC1═CC═C(Cl)C═C1)C═3SC(C)═NC═3C4═CC═C(F)C═C4 |
| Example 1788 | N2═C(SC═C2C(═O)NCCCN1CCCC1═O)C═3SC(C)═NC═3C4═CC═C(F)C═C4 |
| Example 1789 | N2═C(SC═C2C(═O)NCC1═CC═C(F)C═C1F)C═3SC(C)═NC═3C4═CC═C(F)C═C4 |
| Example 1790 | N2═C(SC═C2C(═O)NCC1═CC═C(F)C(F)═C1)C═3SC(C)═NC═3C4═CC═C(F)C═C4 |
| Example 1791 | N2═C(SC═C2C(═O)NCC1═CC(F)═CC(F)═C1)C═3SC(C)═NC═3C4═CC═C(F)C═C4 |
| Example 1792 | N2═C(SC═C2C(═O)NCCCN1CCOCC1)C═3SC(C)═NC═3C4═CC═C(F)C═C4 |
| Example 1793 | N1═C(SC═C1C(═O)NCCN(C(C)C)C(C)C)C═2SC(C)═NC═2C3═CC═C(F)C═C3 |
| Example 1794 | N2═C(SC═C2C(═O)NC(C)CCC1═CC═CC═C1)C═3SC(C)═NC═3C4═CC═C(F)C═C4 |
| Example 1795 | N2═C(SC═C2C(═O)NCC1═CC═C(C(C)C)C═C1)C═3SC(C)═NC═3C4═CC═C(F)C═C4 |
| Example 1796 | N3═C(SC═C3C(═O)NCC2═CC═C1OCOC1═C2)C═4SC(C)═NC═4C5═CC═C(F)C═C5 |
| Example 1797 | N2═C(SC═C2C(═O)NCCC═1C═CC═C(OC)C═1)C═3SC(C)═NC═3C4═CC═C(F)C═C4 |
| Example 1798 | N2═C(SC═C2C(═O)N[C@H](CO)CC1═CC═CC═C1)C═3SC(C)═NC═3C4═CC═C(F)C═C4 |
| Example 1799 | N2═C(SC═C2C(═O)NCCC1═CC═C(OC)C═C1)C═3SC(C)═NC═3C4═CC═C(F)C═C4 |
| Example 1800 | N2═C(SC═C2C(═O)NCCC1═CC═CC═C1OC)C═3SC(C)═NC═3C4═CC═C(F)C═C4 |
| Example 1801 | N2═C(SC═C2C(═O)NCC1═CC═CC═C1OCC)C═3SC(C)═NC═3C4═CC═C(F)C═C4 |
| Example 1802 | N2═C(SC═C2C(═O)NCCC1═CC═CC═C1Cl)C═3SC(C)═NC═3C4═CC═C(F)C═C4 |
| Example 1803 | N2═C(SC═C2C(═O)NCCC1═CC═C(Cl)C═C1)C═3SC(C)═NC═3C4═CC═C(F)C═C4 |
| Example 1804 | N2═C(SC═C2C(═O)NCCC1═CC═CC(Cl)═C1)C═3SC(C)═NC═3C4═CC═C(F)C═C4 |
| Example 1805 | N2═C(SC═C2C(═O)NC1CC(C)(C)NC(C1)(C)C)C═3SC(C)═NC═3C4═CC═C(F)C═C4 |
| Example 1806 | N1═C(SC═C1C(═O)NC(CCCN(CC)CC)C)C═2SC(C)═NC═2C3═CC═C(F)C═C3 |
| Example 1807 | N2═C(SC═C2C(═O)NCC1═CC═C(F)C(Cl)═C1)C═3SC(C)═NC═3C4═CC═C(F)C═C4 |
| Example 1808 | N1═C(SC═C1C(═O)NCCNC(OC(C)(C)C)═O)C═2SC(C)═NC═2C3═CC═C(F)C═C3 |
| Example 1809 | [H]N4C═C(CCNC(═O)C3═CSC(C═1SC(C)═NC═1C2═CC═C(F)C═C2)═N3)C5═CC═CC═C45 |
| Example 1810 | N2═C(SC═C2C(═O)NCC1═CC═C(C(C)(C)C)C═C1)C═3SC(C)═NC═3C4═CC═C(F)C═C4 |
| Example 1811 | N2═C(SC═C2C(═O)NCCCN(C)C1═CC═CC═C1)C═3SC(C)═NC═3C4═CC═C(F)C═C4 |
| Example 1812 | N2═C(SC═C2C(═O)NCC1(O)CCCCC1)C═3SC(C)═NC═3C4═CC═C(F)C═C4 |
| Example 1813 | N2═C(SC═C2C(═O)NCC═1C═C(OC)C═C(C═1)OC)C═3SC(C)═NC═3C4═CC═C(F)C═C4 |
| Example 1814 | N2═C(SC═C2C(═O)NCC1═CC═C(OC)C(═C1)OC)C═3SC(C)═NC═3C4═CC═C(F)C═C4 |
| Example 1815 | N2═C(SC═C2C(═O)NCC(═O)C1═CC═CC═C1)C═3SC(C)═NC═3C4═CC═C(F)C═C4 |
| Example 1816 | N2═C(SC═C2C(═O)NC1CCN(C(OCC)═O)CC1)C═3SC(C)═NC═3C4═CC═C(F)C═C4 |
| Example 1817 | N1═C(SC═C1C(═O)NCCCNC(OC(C)(C)C)═O)C═2SC(C)═NC═2C3═CC═C(F)C═C3 |
| Example 1818 | N2═C(SC═C2C(═O)NCC1═CC═C(C═C1)C(F)(F)F)C═3SC(C)═NC═3C4═CC═C(F)C═C4 |
| Example 1819 | N2═C(SC═C2C(═O)NCC1═CC═CC(═C1)C(F)(F)F)C═3SC(C)═NC═3C4═CC═C(F)C═C4 |
| Example 1820 | N2═C(SC═C2C(═O)NCC1═CC═C(Cl)C(Cl)═C1)C═3SC(C)═NC═3C4═CC═C(F)C═C4 |
| Example 1821 | N2═C(SC═C2C(═O)NCC1═CC═C(Cl)C═C1Cl)C═3SC(C)═NC═3C4═CC═C(F)C═C4 |
| Example 1822 | N3═C(SC═C3C(═O)NC2CCN(CC1═CC═CC═C1)C2)C═4SC(C)═NC═4C5═CC═C(F)C═C5 |
| Example 1823 | N2═C(SC═C2C(═O)NCCC═1C(═CC═C(OC)C═1)OC)C═3SC(C)═NC═3C4═CC═C(F)C═C4 |
| Example 1824 | N2═C(SC═C2C(═O)NCC1═CC═C(N(C)C)C═C1)C═3SC(C)═NC═3C4═CC═C(F)C═C4 |
| Example 1825 | N2═C(SC═C2C(═O)NCC1═CC═C(OC)C(═C1)O)C═3SC(C)═NC═3C4═CC═C(F)C═C4 |
| Example 1826 | N3═C(SC═C3C(═O)NC2CCN(CC1═CC═CC═C1)CC2)C═4SC(C)═NC═4C5═CC═C(F)C═C5 |
| Example 1827 | N2═C(SC═C2C(═O)NCC1═CC═CC═C1OC(F)(F)F)C═3SC(C)═NC═3C4═CC═C(F)C═C4 |
| Example 1828 | N2═C(SC═C2C(═O)NCCC1═CC═C(C═C1)S(═O)(═O)N)C═3SC(C)═NC═3C4═CC═C(F)C═C4 |
| Example 1829 | N2═C(SC═C2C(═O)NCC(C1═CC═C(OC)C═C1)═O)C═3SC(C)═NC═3C4═CC═C(F)C═C4 |
| Example 1830 | N3═C(SC═C3C(═O)NCCC2═CC═C1OCOC1═C2)C═4SC(C)═NC═4C5═CC═C(F)C═C5 |
| Example 1831 | N3═C(SC═C3C(═O)NCCC(C1═CC═CC═C1)C2═CC═CC═C2)C═4SC(C)═NC═4C5═CC═C(F)C═C5 |
| Example 1832 | N2═C(SC═C2C(═O)NCCNS(C1═CC═C(C)C═C1)(═O)═O)C═3SC(C)═NC═3C4═CC═C(F)C═C4 |

Examples Prepared with 2° Amines:

| | |
|---|---|
| Example 1833 | N1═C(SC═C1C(═O)O)C═2SC(C)═NC═2C3═CC═C(F)C═C3 |
| Example 1834 | N1═C(SC═C1C(═O)N(C)C)C═2SC(C)═NC═2C3═CC═C(F)C═C3 |
| Example 1835 | N1═C(SC═C1C(═O)N(CC)C)C═2SC(C)═NC═2C3═CC═C(F)C═C3 |
| Example 1836 | N1═C(SC═C1C(═O)N(C)CC#C)C═2SC(C)═NC═2C3═CC═C(F)C═C3 |
| Example 1837 | N1═C(SC═C1C(═O)N(CC)CC)C═2SC(C)═NC═2C3═CC═C(F)C═C3 |
| Example 1838 | N1═C(SC═C1C(═O)N(CCC)C)C═2SC(C)═NC═2C3═CC═C(F)C═C3 |
| Example 1839 | N1═C(SC═C1C(═O)N(C)CCO)C═2SC(C)═NC═2C3═CC═C(F)C═C3 |
| Example 1840 | N1═C(SC═C1C(═O)N(C)CCC#N)C═2SC(C)═NC═2C3═CC═C(F)C═C3 |
| Example 1841 | N2═C(SC═C2C(═O)N1CCOCC1)C═3SC(C)═NC═3C4═CC═C(F)C═C4 |
| Example 1842 | N1═C(SC═C1C(═O)N(C)CC(C)C)C═2SC(C)═NC═2C3═CC═C(F)C═C3 |
| Example 1843 | N1═C(SC═C1C(═O)N(CC)C(C)C)C═2SC(C)═NC═2C3═CC═C(F)C═C3 |
| Example 1844 | N1═C(SC═C1C(═O)N(CCCC)C)C)═2SC(C)═NC═2C3═CC═C(F)C═C3 |
| Example 1845 | N1═C(SC═C1C(═O)N(CC)CCO)C═2SC(C)═NC═2C3═CC═C(F)C═C3 |
| Example 1846 | N2═C(SC═C2C(═O)N1CSCC1)C═3SC(C)═NC═3C4═CC═C(F)C═C4 |
| Example 1847 | N2═C(SC═C2C(═O)N1CCCCC1C)C═3SC(C)═NC═3C4═CC═C(F)C═C4 |
| Example 1848 | N2═C(SC═C2C(═O)N1CCC(C)CC1)C═3SC(C)═NC═3C4═CC═C(F)C═C4 |
| Example 1849 | N2═C(SC═C2C(═O)N1CCN(C)CC1)C═3SC(C)═NC═3C4═CC═C(F)C═C4 |
| Example 1850 | N2═C(SC═C2C(═O)N1CCCC1CO)C)═3SC(C)═NC═3C4═CC═C(F)C═C4 |
| Example 1851 | N2═C(SC═C2C(═O)N1CCC[C@@H]1CO)C═3SC(C)═NC═3C4═CC═C(F)C═C4 |
| Example 1852 | N1═C(SC═C1C(═O)N(CCCC)CC)C═2SC(C)═NC═2C3═CC═C(F)C═C3 |
| Example 1853 | N1═C(SC═C1C(═O)N(CCC)CCC)C═2SC(C)═NC═2C3═CC═C(F)C═C3 |
| Example 1854 | N2═C(SC═C2C(═O)N1CCSCC1)C═3SC(C)═NC═3C4═CC═C(F)C═C4 |
| Example 1855 | N1═C(SC═C1C(═O)N(CCO)CCO)C═2SC(C)═NC═2C3═CC═C(F)C═C3 |
| Example 1856 | N2═C(SC═C2C(═O)N1CC(C)CC(C1)C)C═3SC(C)═NC═3C4═CC═C(F)C═C4 |
| Example 1857 | N2═C(SC═C2C(═O)N(C)C1CCCCC1)C═3SC(C)═NC═3C4═CC═C(F)C═C4 |
| Example 1858 | N2═C(SC═C2C(═O)N1C(CCCC1C)C)C═3SC(C)═NC═3C4═CC═C(F)C═C4 |
| Example 1859 | N2═C(SC═C2C(═O)N1CCC(N(C)C)C1)C═3SC(C)═NC═3C4═CC═C(F)C═C4 |
| Example 1860 | N2═C(SC═C2C(═O)N1C[C@@H](C)N[C@H](C1)C)C═3SC(C)═NC═3C4═CC═C(F)C═C4 |
| Example 1861 | N2═C(SC═C2C(═O)N1CCCC(CO)C1)C═3SC(C)═NC═3C4═CC═C(F)C═C4 |
| Example 1862 | N2═C(SC═C2C(═O)N1CCC[C@H]1COC)C═3SC(C)═NC═3C4═CC═C(F)C═C4 |
| Example 1863 | N2═C(SC═C2C(═O)N1CC(C)OC(C1)C)C═3SC(C)═NC═3C4═CC═C(F)C═C4 |
| Example 1864 | N2═C(SC═C2C(═O)N1CCCCC1CO)C═3SC(C)═NC═3C4═CC═C(F)C═C4 |
| Example 1865 | N1═C(SC═C1C(═O)N(CC)CCCCO)C═2SC(C)═NC═2C3═CC═C(F)C═C3 |
| Example 1866 | N3═C(SC═C3C(═O)N1CC2═CC═CC═C2C1)C═4SC(C)═NC═4C5═CC═C(F)C═C5 |
| Example 1867 | N2═C(SC═C2C(═O)N(C)CC1═CC═CC═C1)C═3SC(C)═NC═3C4═CC═C(F)C═C4 |
| Example 1868 | N2═C(SC═C2C(═O)N(CC)C1CCCCC1)C═3SC(C)═NC═3C4═CC═C(F)C═C4 |
| Example 1869 | N2═C(SC═C2C(═O)N1CCC(C(N)═O)CC1)C═3SC(C)═NC═3C4═CC═C(F)C═C4 |
| Example 1870 | N2═C(SC═C2C(═O)N1CC[C@H](NC(C)═O)C1)C═3SC(C)═NC═3C4═CC═C(F)C═C4 |
| Example 1871 | N2═C(SC═C2C(═O)N1CC[C@H](NC(C)═O)C1)C═3SC(C)═NC═3C4═CC═C(F)C═C4 |
| Example 1872 | N2═C(SC═C2C(═O)N1CCCC(C(N)═O)C1)C═3SC(C)═NC═3C4═CC═C(F)C═C4 |
| Example 1873 | N2═C(SC═C2C(═O)N1CCCCC1CCO)C═3SC(C)═NC═3C4═CC═C(F)C═C4 |
| Example 1874 | N3═C(SC═C3C(═O)N1CC2═CC═CC═C2CC1)C═4SC(C)═NC═4C5═CC═C(F)C═C5 |
| Example 1875 | N2═C(SC═C2C(═O)N(CC)CC1═CC═CC═C1)C═3SC(C)═NC═3C4═CC═C(F)C═C4 |
| Example 1876 | N2═C(SC═C2C(═O)N(CC)CC1═CC═CC═C1)C═3SC(C)═NC═3C4═CC═C(F)C═C4 |
| Example 1877 | N2═C(SC═C2C(═O)N(C)CCC1═CC═CC═C1)C═3SC(C)═NC═3C4═CC═C(F)C═C4 |
| Example 1878 | N2═C(SC═C2C(═O)N(C)CCC1═CC═CC═N1)C═3SC(C)═NC═3C4═CC═C(F)C═C4 |
| Example 1879 | N2═C(SC═C2C(═O)N(C)CCC1═CC═CC═N1)C═3SC(C)═NC═3C4═CC═C(F)C═C4 |
| Example 1880 | N3═C(SC═C3C(═O)N1C2CCCCC2CCC1)C═4SC(C)═NC═4C5═CC═C(F)C═C5 |
| Example 1881 | N2═C(SC═C2C(═O)N(CC═C)C1CCCCC1)C═3SC(C)═NC═3C4═CC═C(F)C═C4 |
| Example 1882 | N2═C(SC═C2C(═O)N1CCC(C(OC)═O)CC1)C═3SC(C)═NC═3C4═CC═C(F)C═C4 |
| Example 1883 | N2═C(SC═C2C(═O)N(C(C)C)CC1═CC═CC═C1)C═3SC(C)═NC═3C4═CC═C(F)C═C4 |
| Example 1884 | N2═C(SC═C2C(═O)N(CCO)CC1═CC═CC═C1)C═3SC(C)═NC═3C4═CC═C(F)C═C4 |
| Example 1885 | N2═C(SC═C2C(═O)N(C)CC(O)C1═CC═CC═C1)C═3SC(C)═NC═3C4═CC═C(F)C═C4 |
| Example 1886 | N2═C(SC═C2C(═O)N1CCC(O)(O)CC1)C═3SC(C)═NC═3C4═CC═C(F)C═C4 |
| Example 1887 | N2═C(SC═C2C(═O)N1CCC(C(OCC)═O)CC1)C═3SC(C)═NC═3C4═CC═C(F)C═C4 |
| Example 1888 | N2═C(SC═C2C(═O)N1CCCC(C(OCC)═O)C1)C═3SC(C)═NC═3C4═CC═C(F)C═C4 |
| Example 1889 | N2═C(SC═C2C(═O)N1CCN(C(OCC)═O)CC1)C═3SC(C)═NC═3C4═CC═C(F)C═C4 |
| Example 1890 | N2═C(SC═C2C(═O)N(CCC#N)CC1═CC═CC═C1)C═3SC(C)═NC═3C4═CC═C(F)C═C4 |
| Example 1891 | N2═C(SC═C2C(═O)N(CCC#N)CC1═CC═CN═C1)C═3SC(C)═NC═3C4═CC═C(F)C═C4 |
| Example 1892 | N3═C(SC═C3C(═O)N1CCN(CC1)C2═CC═CC═C2)C═4SC(C)═NC═4C5═CC═C(F)C═C5 |
| Example 1893 | N3═C(SC═C3C(═O)N1CCN(CC1)C2═CC═CC═N2)C═4SC(C)═NC═4C5═CC═C(F)C═C5 |
| Example 1894 | N2═C(SC═C2C(═O)N(CCCC)CC1═CC═CC═C1)C═3SC(C)═NC═3C4═CC═C(F)C═C4 |
| Example 1895 | N2═C(SC═C2C(═O)N([C@H](C)C1═CC═CC═C1)CCO)C═3SC(C)═NC═3C4═CC═C(F)C═C4 |
| Example 1896 | N2═C(SC═C2C(═O)N(CCCO)CC1═CC═CC═N1)C═3SC(C)═NC═3C4═CC═C(F)C═C4 |
| Example 1897 | N2═C(SC═C2C(═O)N(C)CC(O)C1═CC═C(O)C═C1)C═3SC(C)═NC═3C4═CC═C(F)C═C4 |
| Example 1898 | N3═C(SC═C3C(═O)N2CCN(C1CCCCC1)CC2)C═4SC(C)═NC═4C5═CC═C(F)C═C5 |
| Example 1899 | N3═C(SC═C3C(═O)N2CCC(CC1═CC═CC═C1)CC2)C═4SC(C)═NC═4C5═CC═C(F)C═C5 |
| Example 1900 | N3═C(SC═C3C(═O)N2CCN(CC1═CC═CC═C1)CC2)C═4SC(C)═NC═4C5═CC═C(F)C═C5 |
| Example 1901 | N3═C(SC═C3C(═O)N2CCCN(CC1═CC═CC═C1)CC2)C═4SC(C)═NC═4C5═CC═C(F)C═C5 |
| Example 1902 | N3═C(SC═C3C(═O)N2CCN(C1═CC═C(O)C═C1)CC2)C═4SC(C)═NC═4C5═CC═C(F)C═C5 |
| Example 1903 | N2═C(SC═C2C(═O)N(CCN(C)C)CC1═CC═CC═C1)C═3SC(C)═NC═3C4═CC═C(F)C═C4 |
| Example 1904 | N3═C(SC═C3C(═O)N2CCN(C1═CC═CC═C1F)CC2)C═4SC(C)═NC═4C5═CC═C(F)C═C5 |
| Example 1905 | N3═C(SC═C3C(═O)N2CCN(C1═CC═C(F)C═C1)CC2)C═4SC(C)═NC═4C5═CC═C(F)C═C5 |
| Example 1906 | N3═C(SC═C3C(═O)N2CCN(CC1CCCCC1)CC2)C═4SC(C)═NC═4C5═CC═C(F)C═C5 |
| Example 1907 | N2═C(SC═C2C(═O)N(C)C[C@H](O)C1═CC═C(O)C(O)═C1)C═3SC(C)═NC═3C4═CC═C(F)C═C4 |
| Example 1908 | N2═C(SC═C2C(═O)N1C(CC(OCC)═O)C(NCC1)═O)C═3SC(C)═NC═3C4═CC═C(F)C═C4 |
| Example 1909 | N2═C(SC═C2C(═O)N1CCN(C(OC(C)(C)C)═O)CC1)C═3SC(C)═NC═3C4═CC═C(F)C═C4 |
| Example 1910 | N2═C(SC═C2C(═O)N1CCC(NC(OC(C)(C)C)═O)C1)C═3SC(C)═NC═3C4═CC═C(F)C═C4 |
| Example 1911 | N3═C(SC═C3C(═O)N2CCN(C1═CC═CC═C1C#N)CC2)C═4SC(C)═NC═4C5═CC═C(F)C═C5 |
| Example 1912 | N3═C(SC═C3C(═O)N2CCN(C1═CC═C(C#N)C═N1)CC2)C═4SC(C)═NC═4C5═CC═C(F)C═C5 |
| Example 1913 | N3═C(SC═C3C(═O)N2CCN(C═1C═CC═C(C)C═1C)CC2)C═4SC(C)═NC═4C5═CC═C(F)C═C5 |
| Example 1914 | N3═C(SC═C3C(═O)N2CCN(C═1C═CC═C(C)C═1C)CC2)C═4SC(C)═NC═4C5═CC═C(F)C═C5 |
| Example 1915 | N3═C(SC═C3C(═O)N1CC(C)N(CC1)C2═CC═CC(C)═C2)C═4SC(C)═NC═4C5═CC═C(F)C═C5 |
| Example 1916 | N3═C(SC═C3C(═O)N2CCN(CCC1═CC═CC═C1)CC2)C═4SC(C)═NC═4C5═CC═C(F)C═C5 |
| Example 1917 | N3═C(SC═C3C(═O)N2CCN(C1═CC═C(C)C(═C1)C)CC2)C═4SC(C)═NC═4C5═CC═C(F)C═C5 |
| Example 1918 | N3═C(SC═C3C(═O)N2CCN(C1═CC═C(C)C═C1C)CC2)C═4SC(C)═NC═4C5═CC═C(F)C═C5 |
| Example 1919 | N3═C(SC═C3C(═O)N2CCN(C═1C(═CC═C(C)C═1)C)CC2)C═4SC(C)═NC═4C5═CC═C(F)C═C5 |
| Example 1920 | N3═C(SC═C3C(═O)N2CCN(C1═CC═C(OC)C═C1)CC2)C═4SC(C)═NC═4C5═CC═C(F)C═C5 |
| Example 1921 | N3═C(SC═C3C(═O)N2CCN(C1═CC═CC═C1OC)CC2)C═4SC(C)═NC═4C5═CC═C(F)C═C5 |
| Example 1922 | N3═C(SC═C3C(═O)N2CCN(C═1C═CC═C(OC)C═1)CC2)C═4SC(C)═NC═4C5═CC═C(F)C═C5 |
| Example 1923 | N2═C(SC═C2C(═O)N(C)CCC1═CC═C(OC)C(OC)═C1)C═3SC(C)═NC═3C4═CC═C(F)C═C4 |
| Example 1924 | N3═C(SC═C3C(═O)N2CCN(C═1C═CC═C(Cl)C═1)CC2)C═4SC(C)═NC═4C5═CC═C(F)C═C5 |
| Example 1925 | N3═C(SC═C3C(═O)N2CCN(C1═CC═C(Cl)C═C1)CC2)C═4SC(C)═NC═4C5═CC═C(F)C═C5 |
| Example 1926 | N3═C(SC═C3C(═O)N2CCN(C1═CC═C(Cl)C═C1)CC2)C═4SC(C)═NC═4C5═CC═C(F)C═C5 |
| Example 1927 | N3═C(SC═C3C(═O)N2CCN(C1═CC═C(F)C═C1F)CC2)C═4SC(C)═NC═4C5═CC═C(F)C═C5 |
| Example 1928 | N3═C(SC═C3C(═O)N(CC1═CC═CN═C1)CC2═CC═CN═C2)C═4SC(C)═NC═4C5═CC═C(F)C═C5 |
| Example 1929 | N3═C(SC═C3C(═O)N2CCN(CCN1CCOCC1)CC2)C═4SC(C)═NC═4C5═CC═C(F)C═C5 |
| Example 1930 | N2═C(SC═C2C(═O)N1CCC(N(C)C(═O)OC(C)(C)C)C1)C═3SC(C)═NC═3C4═CC═C(F)C═C4 |
| Example 1931 | N3═C(SC═C3C(═O)N2CCN(C1═CC═C(C(C)═O)C═C1)CC2)C═4SC(C)═NC═4C5═CC═C(F)C═C5 |
| Example 1932 | N2═C(SC═C2C(═O)N(CCN(CC)CC)CC1═CC═CC═C1)C═3SC(C)═NC═3C4═CC═C(F)C═C4 |
| Example 1933 | N3═C(SC═C3C(═O)N2CCN(C1═CC═C([N+]([O−])═O)C═C1)CC2)C═4SC(C)═NC═4C5═CC═C(F)C═C5 |
| Example 1934 | N3═C(SC═C3C(═O)N(C)CC1═CC═CC2═CC═CC═C12)C═4SC(C)═NC═4C5═CC═C(F)C═C5 |
| Example 1935 | N3═C(SC═C3C(═O)N2CCN(C1═CC(═CC═C1C)Cl)CC2)C═4SC(C)═NC═4C5═CC═C(F)C═C5 |
| Example 1936 | N2═C(SC═C2C(═O)N(C)CC1═CC(OC)═C(OC)C(OC)═C1)C═3SC(C)═NC═3C4═CC═C(F)C═C4 |
| Example 1937 | N3═C(SC═C3C(═O)N(CCC1═CC═CC═C1)CC2═CC═CC═C2)C═4SC(C)═NC═4C5═CC═C(F)C═C5 |
| Example 1938 | N3═C(SC═C3C(═O)N(CCC1═CC═CC═C1)CC2═CC═CC═C2)C═4SC(C)═NC═4C5═CC═C(F)C═C5 |
| Example 1939 | N3═C(SC═C3C(═O)N(C)C(CC1═CC═CC═C1)C2═CC═CC═C2)C═4SC(C)═NC═4C5═CC═C(F)C═C5 |
| Example 1940 | N3═C(SC═C3C(═O)N1CCC(O)(CC1)C2═CC═C(Cl)C═C2)C═4SC(C)═NC═4C5═CC═C(F)C═C5 |
| Example 1941 | N2═C(SC═C2C(═O)N(CC1═CC═C(Cl)C═C1Cl)CC #C)C═3SC(C)═NC═3C4═CC═C(F)C═C4 |
| Example 1942 | N3═C(SC═C3C(═O)N(CC)C(CC1═CC═CC═C1)C═2OC═CC═2)C═4SC(C)═NC═4C5═CC═C(F)C═C5 |
| Example 1943 | N2═C(SC═C2C(═O)N1CCN(C(OC(C)(C)C)═O)C[C@H]1CO)C═3SC(C)═NC═3C4═CC═C(F)C═C4 |
| Example 1944 | N3═C(SC═C3C(═O)N2CCN(C1═CC═C(C(C)(C)C)C═C1)CC2)C═4SC(C)═NC═4C5═CC═C(F)C═C5 |
| Example 1945 | N4═C(SC═C4C(═O)N3CCN(CC2═CC═C1OCOC1═C2)CC3)C═5SC(C)═NC═5C6═CC═C(F)C═C6 |
| Example 1946 | N3═C(SC═C3C(═O)N1CCC═2C═C(OC)C(═CC═2C1)OC)C═4SC(C)═NC═4C5═CC═C(F)C═C5 |
| Example 1947 | N3═C(SC═C3C(═O)N2CCN(C1═CC═C(C(F)(F)F)C═C1)CC2)C═4SC(C)═NC═4C5═CC═C(F)C═C5 |
| Example 1948 | N3═C(SC═C3C(═O)N2CCN(C═1C═CC═C(C(F)(F)F)C═1)CC2)C═4SC(C)═NC═4C5═CC═C(F)C═C5 |
| Example 1949 | N3═C(SC═C3C(═O)N2CCN(C1═CC═C(Cl)C(═C1)Cl)CC2)C═4SC(C)═NC═4C5═CC═C(F)C═C5 |
| Example 1950 | N3═C(SC═C3C(═O)N2CCN(C1═CC═C(Cl)C(═C1)Cl)CC2)C═4SC(C)═NC═4C5═CC═C(F)C═C5 |
| Example 1951 | N3═C(SC═C3C(═O)N2CCN(C1═CC═C(C(F)(F)F)C═N1)CC2)C═4SC(C)═NC═4C5═CC═C(F)C═C5 |
| Example 1952 | N3═C(SC═C3C(═O)N2CCN(C1═CC═CC═C1Cl)CC2)C═4SC(C)═NC═4C5═CC═C(F)C═C5 |
| Example 1953 | N3═C(SC═C3C(═O)N2CCN(C1═CC═C(OC)C(═C1)OC)CC2)C═4SC(C)═NC═4C5═CC═C(F)C═C5 |
| Example 1954 | N3═C(SC═C3C(═O)N2CCCC(C1═CC═C(C(F)(F)F)C═C1)C2)C═4SC(C)═NC═4C5═CC═C(F)C═C5 |

Examples Prepared with Anilines:

| | |
|---|---|
| Example 1955 | N2═C(SC═C2C(═O)NC1═CC═CC═C1)C═3SC(C)═NC═3C4═CC═C(F)C═C4 |
| Example 1956 | N2═C(SC═C2C(═O)NC═1C═CC═C(C)C═1)C═3SC(C)═NC═3C4═CC═C(F)C═C4 |
| Example 1957 | N2═C(SC═C2C(═O)NC1═CC═C(C)C═C1)C═3SC(C)═NC═3C4═CC═C(F)C═C4 |
| Example 1958 | N2═C(SC═C2C(═O)NC1═CC═CC═C1C)C═3SC(C)═NC═3C4═CC═C(F)C═C4 |
| Example 1959 | N2═C(SC═C2C(═O)NC1═CC═C(C)C═N1)C═3SC(C)═NC═3C4═CC═C(F)C═C4 |
| Example 1960 | N2═C(SC═C2C(═O)NC═1C═CC═C(C)N═1)C═3SC(C)═NC═3C4═CC═C(F)C═C4 |
| Example 1961 | N2═C(SC═C2C(═O)NC═1N═CC═C(C)C═1)C═3SC(C)═NC═3C4═CC═C(F)C═C4 |
| Example 1962 | N2═C(SC═C2C(═O)NC1═NC═CC═C1C)C═3SC(C)═NC═3C4═CC═C(F)C═C4 |
| Example 1963 | N2═C(SC═C2C(═O)NC1═CC═CC═C1F)C═3SC(C)═NC═3C4═CC═C(F)C═C4 |
| Example 1964 | N2═C(SC═C2C(═O)NC1═CC═C(F)C═C1)C═3SC(C)═NC═3C4═CC═C(F)C═C4 |
| Example 1965 | N2═C(SC═C2C(═O)NC═1C(═CC═C(C)C═1)C)C═3SC(C)═NC═3C4═CC═C(F)C═C4 |
| Example 1966 | N2═C(SC═C2C(═O)NC1═CC═C(C)C(═C1)C)C═3SC(C)═NC═3C4═CC═C(F)C═C4 |
| Example 1967 | N2═C(SC═C2C(═O)NC1═CC═CC═C1CC)C═3SC(C)═NC═3C4═CC═C(F)C═C4 |
| Example 1968 | N2═C(SC═C2C(═O)NC1═CC═CC═C1CC)C═3SC(C)═NC═3C4═CC═C(F)C═C4 |
| Example 1969 | N2═C(SC═C2C(═O)NC═1C═CC═(C)C═1C)C═3SC(C)═NC═3C4═CC═C(F)C═C4 |
| Example 1970 | N2═C(SC═C2C(═O)NC1═CC═C(C)C═C1C)C═3SC(C)═NC═3C4═CC═C(F)C═C4 |
| Example 1971 | N2═C(SC═C2C(═O)NC═1C═C(C═C(C)C═1)C)C═3SC(C)═NC═3C4═CC═C(F)C═C4 |
| Example 1972 | N2═C(SC═C2C(═O)NC═1C═CC═C(OC)C═1)C═3SC(C)═NC═3C4═CC═C(F)C═C4 |
| Example 1973 | N2═C(SC═C2C(═O)NC1═CC═C(OC)C═C1)C═3SC(C)═NC═3C4═CC═C(F)C═C4 |
| Example 1974 | N2═C(SC═C2C(═O)NC1═CC═CC═C1OC)C═3SC(C)═NC═3C4═CC═C(F)C═C4 |
| Example 1975 | N2═C(SC═C2C(═O)NC1═CN═CC═C1OC)C═3SC(C)═NC═3C4═CC═C(F)C═C4 |
| Example 1976 | N2═C(SC═C2C(═O)NC1═CC═C(C)C(═C1)F)C═3SC(C)═NC═3C4═CC═C(F)C═C4 |
| Example 1977 | N2═C(SC═C2C(═O)NC1═C(C)C(═CC═C1)F)C═3SC(C)═NC═3C4═CC═C(F)C═C4 |
| Example 1978 | N2═C(SC═C2C(═O)NC1═CC═(F)═CC(F)═C1)C═3SC(C)═NC═3C4═CC═C(F)C═C4 |
| Example 1979 | N2═C(SC═C2C(═O)NC1═CC═C(F)C═C1F)C═3SC(C)═NC═3C4═CC═C(F)C═C4 |
| Example 1980 | N2═C(SC═C2C(═O)NC1═CC═C(F)C═C1F)C═3SC(C)═NC═3C4═CC═C(F)C═C4 |
| Example 1981 | N3═C(SC═C3C(═O)NC2═CC═C1CCCC1═C2)C═4SC(C)═NC═4C5═CC═C(F)C═C5 |
| Example 1982 | N2═C(SC═C2C(═O)NC1═CC═CC═C1C(C)C)C═3SC(C)═NC═3C4═CC═C(F)C═C4 |
| Example 1983 | N2═C(SC═C2C(═O)NC═1C═CC═C(C(C)═O)C═1)C═3SC(C)═NC═3C4═CC═C(F)C═C4 |
| Example 1984 | N2═C(SC═C2C(═O)NC1═CC═C(C(C)═O)C═C1)C═3SC(C)═NC═3C4═CC═C(F)C═C4 |
| Example 1985 | N2═C(SC═C2C(═O)NC1═C(C═CC═C1CC)C)C═3SC(C)═NC═3C4═CC═C(F)C═C4 |
| Example 1986 | N2═C(SC═C2C(═O)NC1═C(C═C(C)C═C1C)C)C═3SC(C)═NC═3C4═CC═C(F)C═C4 |
| Example 1987 | N3═C(SC═C3C(═O)NC2═CC═C1OCOC1═C2)C═4SC(C)═NC═4C5═CC═C(F)C═C5 |
| Example 1988 | N2═C(SC═C2C(═O)NC1═CC═C(OC)C═C1C)C═3SC(C)═NC═3C4═CC═C(F)C═C4 |
| Example 1989 | N2═C(SC═C2C(═O)NC═1C═CC═C(OCC)C═1)C═3SC(C)═NC═3C4═CC═C(F)C═C4 |
| Example 1990 | N2═C(SC═C2C(═O)NC1═CC═CC═C1OCC)C═3SC(C)═NC═3C4═CC═C(F)C═C4 |
| Example 1991 | N2═C(SC═C2C(═O)NC1═CC═CC═C1SC)C═3SC(C)═NC═3C4═CC═C(F)C═C4 |
| Example 1992 | N2═C(SC═C2C(═O)NC1═C(C)C(═CC═C1)Cl)C═3SC(C)═NC═3C4═CC═C(F)C═C4 |
| Example 1993 | N2═C(SC═C2C(═O)NC1═CC═C(C)C(═C1)Cl)C═3SC(C)═NC═3C4═CC═C(F)C═C4 |
| Example 1994 | N3═C(SC═C3C(═O)NC2═CN═C1C═CC═CC1═C2)C═4SC(C)═NC═4C5═CC═C(F)C═C5 |
| Example 1995 | N2═C(SC═C2C(═O)NC1═CC(F)═C(F)C═C1F)C═3SC(C)═NC═3C4═CC═C(F)C═C4 |
| Example 1996 | N3═C(SC═C3C(═O)NC1CCCC2═CC═CC═C12)C═4SC(C)═NC═4C5═CC═C(F)C═C5 |
| Example 1997 | N2═C(SC═C2C(═O)NC1═CC═CC═C1C(C)(C)C)C═3SC(C)═NC═3C4═CC═C(F)C═C4 |
| Example 1998 | N2═C(SC═C2C(═O)NC1═C(C═CC═C1C(C)C)C)C═3SC(C)═NC═3C4═CC═C(F)C═C4 |
| Example 1999 | N3═C(SC═C3C(═O)NC2═CC═C1N═CSC1═C2)C═4SC(C)═NC═4C5═CC═C(F)C═C5 |
| Example 2000 | N2═C(SC═C2C(═O)NC1═CC═C(NC(C)═O)C═C1)C═3SC(C)═NC═3C4═CC═C(F)C═C4 |
| Example 2001 | N2═C(SC═C2C(═O)NC1═CC═C(NC(C)═O)N═C1)C═3SC(C)═NC═3C4═CC═C(F)C═C4 |
| Example 2002 | N3═C(SC═C3C(═O)NC2═CC═C1OCCOC1═C2)C═4SC(C)═NC═4C5═CC═C(F)C═C5 |
| Example 2003 | N2═C(SC═C2C(═O)NC1═CC═C(OC)C(═C1)OC)C═3SC(C)═NC═3C4═CC═C(F)C═C4 |
| Example 2004 | N3═C(SC═C3C(═O)NC1═CC═CC═C1N2C═CC═C2)C═4SC(C)═NC═4C5═CC═C(F)C═C5 |
| Example 2005 | N2═C(SC═C2C(═O)NC1═CC═C(C═C1)C(F)(F)F)C═3SC(C)═NC═3C4═CC═C(F)C═C4 |
| Example 2006 | N2═C(SC═C2C(═O)NC1═CC═CC(═C1)C(F)(F)F)C═3SC(C)═NC═3C4═CC═C(F)C═C4 |
| Example 2007 | N2═C(SC═C2C(═O)NC1═CC═C(Cl)C(Cl)═C1)C═3SC(C)═NC═3C4═CC═C(F)C═C4 |
| Example 2008 | N2═C(SC═C2C(═O)NC1═C(C═CC═C1C)C(C)(C)C)C═3SC(C)═NC═3C4═CC═C(F)C═C4 |
| Example 2009 | N2═C(SC═C2C(═O)NC1═CC═C(N(C)C)C═C1)C═3SC(C)═NC═3C4═CC═C(F)C═C4 |
| Example 2010 | N3═C(SC═C3C(═O)NC1═CC═CC═C1C2═CC═CC═C2)C═4SC(C)═NC═4C5═CC═C(F)C═C5 |
| Example 2011 | N2═C(SC═C2C(═O)NC1═CC═C(OC(F)(F)F)C═C1)C═3SC(C)═NC═3C4═CC═C(F)C═C4 |
| Example 2012 | N2═C(SC═C2C(═O)NC1═C(C═CC═C1C(C)C)C(C)C)C═3SC(C)═NC═3C4═CC═C(F)C═C4 |
| Example 2013 | N2═C(SC═C2C(═O)NC1═CC═C(F)C═C1C(F)(F)F)C═3SC(C)═NC═3C4═CC═C(F)C═C4 |
| Example 2014 | N2═C(SC═C2C(═O)NC1═CC(═CC═C1F)C(F)(F)F)C═3SC(C)═NC═3C4═CC═C(F)C═C4 |
| Example 2015 | N2═C(SC═C2C(═O)NC1═CC═C(F)C(═C1)C(F)(F)F)C═3SC(C)═NC═3C4═CC═C(F)C═C4 |
| Example 2016 | N2═C(SC═C2C(═O)NC═1C(═CC═C(OCC)C═1)OCC)C═3SC(C)═NC═3C4═CC═C(F)C═C4 |
| Example 2017 | N2═C(SC═C2C(═O)NC1═CC(Cl)═C(Cl)C(Cl)═C1)C═3SC(C)═NC═3C4═CC═C(F)C═C4 |
| Example 2018 | N3═C(SC═C3C(═O)NC1═CC═CC═C1C(═O)C2═CC═CC═C2)C═4SC(C)═NC═4C5═CC═C(F)C═C5 |
| Example 2019 | N3═C(SC═C3C(═O)NC1═CC═CC═C1COC2═CC═CC═C2)C═4SC(C)═NC═4C5═CC═C(F)C═C5 |
| Example 2020 | N3═C(SC═C3C(═O)NC1═CC(═CC═C1OC)C2═CC═CC═C2)C═4SC(C)═NC═4C5═CC═C(F)C═C5 |
| Example 2021 | N3═C(SC═C3C(═O)NC1═NC═CC═C1OCC2═CC═CC═C2)C═4SC(C)═NC═4C5═CC═C(F)C═C5 |
| Example 2022 | N2═C(SC═C2C(═O)NC1═CC(═CC═C1C(F)(F)F)C(F)(F)F)C═3SC(C)═NC═3C4═CC═C(F)C═C4 |
| Example 2023 | N2═C(SC═C2C(═O)NC1═CC(═CC(═C1)C(F)(F)F)C(F)(F)F)C═3SC(C)═NC═3C4═CC═C(F)C═C4 |
| Example 2024 | N2═C(SC═C2C(═O)NC1═CC═C(C═C1C(F)(F)F)C(F)(F)F)C═3SC(C)═NC═3C4═CC═C(F)C═C4 |
| Example 2025 | N1═C(SC═C1C(═O)O)C═2SC(C)═NC═2C3═CC═C(F)C═C3 |

### EXAMPLES 2026-2157

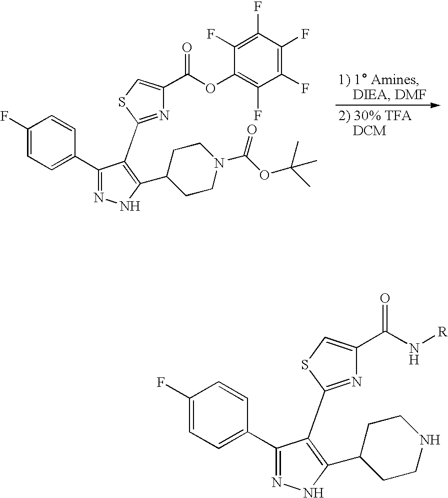

Starting pentafluorophenyl ester was prepared as described in Step 1 of Example 71. Where, 1° amines were selected to afford Examples 2026-2157, which were prepared by General Condition 2, followed by boc deprotection of the piperidine moiety with 30% TFA in DCM (42 μL per well).

| | |
|---|---|
| Example 2026 | [H]N2C(═C(C═1SC═C(C(═O)NCC)N═1)C(═N2)C═3C═CC(═CC═3)F)C4CCNCC4 |
| Example 2027 | [H]N2C(═C(C═1SC═C(C(═O)NCC#C)N═1)C(═N2)C═3C═CC(═CC═3)F)C4CCNCC4 |
| Example 2028 | [H]N3C(═C(C═2SC═C(C(═O)NCC1═CN═CC═C1)N═2)C(═N3)C═4C═CC(═CC═4)F)C5CCNCC5 |
| Example 2029 | [H]N3C(═C(C═2SC═C(C(═O)NCC1CC1)N═2)C(═N3)C═4C═CC(═CC═4)F)C5CCNCC5 |
| Example 2030 | [H]N2N═C(C1═CC═C(C═C1)F)C(═C2C3CCNCC3)C4═NC(═CS4)C(O[H])═O |
| Example 2031 | [H]N2C(═C(C═1SC═C(C(═O)NC)N═1)C(═N2)C3═CC═C(F)C═C3)C4CCNCC4 |
| Example 2032 | [H]N2C(═C(C═1SC═C(C(═O)NCC#N)N═1)C(═N2)C3═CC═C(F)C═C3)C4CCNCC4 |
| Example 2033 | [H]N3C(═C(C═2SC═C(C(═O)NC1CC1)N═2)C(═N3)C4═CC═C(F)C═C4)C5CCNCC5 |
| Example 2034 | [H]N2C(═C(C═1SC═C(C(═O)NCCC)N═1)C(═N2)C3═CC═C(F)C═C3)C4CCNCC4 |
| Example 2035 | [H]N2C(═C(C═1SC═C(C(═O)NCCN)N═1)C(═N2)C3═CC═C(F)C═C3)C4CCNCC4 |
| Example 2036 | [H]N2C(═C(C═1SC═C(C(═O)NCCO)N═1)C(═N2)C3═CC═C(F)C═C3)C4CCNCC4 |
| Example 2037 | [H]N2C(═C(C═1SC═C(C(═O)NCCC#N)N═1)C(═N2)C3═CC═C(F)C═C3)C4CCNCC4 |
| Example 2038 | [H]N3C(═C(C═2SC═C(C(═O)NC1CCC1)N═2)C(═N3)C4═CC═C(F)C═C4)C5CCNCC5 |
| Example 2039 | [H]N2C(═C(C═1SC═C(C(═O)NC(CC)C)N═1)C(═N2)C3═CC═C(F)C═C3)C4CCNCC4 |
| Example 2040 | [H]N2C(═C(C═1SC═C(C(═O)NCC(C)C)N═1)C(═N2)C3═CC═C(F)C═C3)C4CCNCC4 |
| Example 2041 | [H]N2C(═C(C═1SC═C(C(═O)NCCCO)N═1)C(═N2)C3═CC═C(F)C═C3)C4CCNCC4 |
| Example 2042 | [H]N2C(═C(C═1SC═C(C(═O)NC(C)CO)N═1)C(═N2)C3═CC═C(F)C═C3)C4CCNCC4 |
| Example 2043 | [H]N2C(═C(C═1SC═C(C(═O)NCCOC)N═1)C(═N2)C3═CC═C(F)C═C3)C4CCNCC4 |
| Example 2044 | [H]N3C(═C(C═2SC═C(C(═O)NC1CCCC1)N═2)C(═N3)C4═CC═C(F)C═C4)C5CCNCC5 |
| Example 2045 | [H]N2C(═C(C═1SC═C(C(═O)NC(CCC)C)N═1)C(═N2)C3═CC═C(F)C═C3)C4CCNCC4 |
| Example 2046 | [H]N2C(═C(C═1SC═C(C(═O)NCC(CC)C)N═1)C(═N2)C3═CC═C(F)C═C3)C4CCNCC4 |
| Example 2047 | [H]N2C(═C(C═1SC═C(C(═O)NC(C(C)C)C)N═1)C(═N2)C3═CC═C(F)C═C3)C4CCNCC4 |
| Example 2048 | [H]N2C(═C(C═1SC═C(C(═O)NCCC(C)C)N═1)C(═N2)C3═CC═C(F)C═C3)C4CCNCC4 |
| Example 2049 | [H]N2C(═C(C═1SC═C(C(═O)NCCN(C)C)N═1)C(═N2)C3═CC═C(F)C═C3)C4CCNCC4 |
| Example 2050 | [H]N2C(═C(C═1SC═C(C(═O)NC(COC)C)N═1)C(═N2)C3═CC═C(F)C═C3)C4CCNCC4 |
| Example 2051 | [H]N2C(═C(C═1SC═C(C(═O)NC(CC)CO)N═1)C(═N2)C3═CC═C(F)C═C3)C4CCNCC4 |
| Example 2052 | [H]N2C(═C(C═1SC═C(C(═O)NC(CC)CO)N═1)C(═N2)C3═CC═C(F)C═C3)C4CCNCC4 |
| Example 2053 | [H]N2C(═C(C═1SC═C(C(═O)NCCCCO)N═1)C(═N2)C3═CC═C(F)C═C3)C4CCNCC4 |
| Example 2054 | [H]N3C(═C(C═2SC═C(C(═O)NCC═1OC═CC═1)N═2)C(═N3)C4═CC═C(F)C═C4)C5CCNCC5 |
| Example 2055 | [H]N3C(═C(C═2SC═C(C(═O)NC1CCCCC1)N═2)C(═N3)C4═CC═C(F)C═C4)C5CCNCC5 |
| Example 2056 | [H]N3C(═C(C═2SC═C(C(═O)NCC1CCCO1)N═2)C(═N3)C4═CC═C(F)C═C4)C5CCNCC5 |
| Example 2057 | [H]N2C(═C(C═1SC═C(C(═O)NCCC(C)(C)C)N═1)C(═N2)C3═CC═C(F)C═C3)C4CCNCC4 |
| Example 2058 | [H]N2C(═C(C═1SC═C(C(═O)NC(CC(C)C)C)N═1)C(═N2)C3═CC═C(F)C═C3)C4CCNCC4 |
| Example 2059 | [H]N3C(═C(C═2SC═C(C(═O)NC1CONC1═O)N═2)C(═N3)C4═CC═C(F)C═C4)C5CCNCC5 |
| Example 2060 | [H]N3C(═C(C═2SC═C(C(═O)NN1CCOCC1)N═2)C(═N3)C4═CC═C(F)C═C4)C5CCNCC5 |
| Example 2061 | [H]N2C(═C(C═1SC═C(C(═O)NC(C(C)C)CO)N═1)C(═N2)C3═CC═C(F)C═C3)C4CCNCC4 |
| Example 2062 | [H]N3C(═C(C═2SC═C(C(═O)NCC1═CC═CC═C1)N═2)C(═N3)C4═CC═C(F)C═C4)C5CCNCC5 |
| Example 2063 | [H]N3C(═C(C═2SC═C(C(═O)NCC1═CC═CC═N1)N═2)C(═N3)C4═CC═C(F)C═C4)C5CCNCC5 |
| Example 2064 | [H]N3C(═C(C═2SC═C(C(═O)NCC1═CC═NC═C1)N═2)C(═N3)C4═CC═C(F)C═C4)C5CCNCC5 |
| Example 2065 | [H]N3C(═C(C═2SC═C(C(═O)NCC1═CC═CS1)N═2)C(═N3)C4═CC═C(F)C═C4)C5CCNCC5 |
| Example 2066 | [H]N3C(═C(C═2SC═C(C(═O)NC1CCC(C)CC1)N═2)C(═N3)C4═CC═C(F)C═C4)C5CCNCC5 |
| Example 2067 | [H]N3C(═C(C═2SC═C(C(═O)NCC1CCCCC1)N═2)C(═N3)C4═CC═C(F)C═C4)C5CCNCC5 |
| Example 2068 | [H]N3C(═C(C═2SC═C(C(═O)NC1CCCCCC1)N═2)C(═N3)C4═CC═C(F)C═C4)C5CCNCC5 |
| Example 2069 | [H]N3C(═C(C═2SC═C(C(═O)NC1CCCCC1C)N═2)C(═N3)C4═CC═C(F)C═C4)C5CCNCC5 |
| Example 2070 | [H]N3C(═C(C═2SC═C(C(═O)NCCN1CCCC1)N═2)C(═N3)C4═CC═C(F)C═C4)C5CCNCC5 |
| Example 2071 | [H]N3C(═C(C═2SC═C(C(═O)NN1CCN(C)CC1)N═2)C(═N3)C4═CC═C(F)C═C4)C5CCNCC5 |
| Example 2072 | [H]N2C(═C(C═1SC═C(C(═O)NCCN(CC)CC)N═1)C(═N2)C3═CC═C(F)C═C3)C4CCNCC4 |
| Example 2073 | [H]N3C(═C(C═2SC═C(C(═O)NCC═1C═CC═C(C)C═1)N═2)C(═N3)C4═CC═C(F)C═C4)C5CCNCC5 |
| Example 2074 | [H]N3C(═C(C═2SC═C(C(═O)N[C@@H](C)C1═CC═CC═C1)N═2)C(═N3)C4═CC═C(F)C═C4)C5CCNCC5 |
| Example 2075 | [H]N3C(═C(C═2SC═C(C(═O)N[C@H](C)C1═CC═CC═C1)N═2)C(═N3)C4═CC═C(F)C═C4)C5CCNCC5 |
| Example 2076 | [H]N3C(═C(C═2SC═C(C(═O)NCCC1═CC═CC═C1)N═2)C(═N3)C4═CC═C(F)C═C4)C5CCNCC5 |
| Example 2077 | [H]N3C(═C(C═2SC═C(C(═O)NCC1═CC═CC═C1C)N═2)C(═N3)C4═CC═C(F)C═C4)C5CCNCC5 |
| Example 2078 | [H]N3C(═C(C═2SC═C(C(═O)NCC1═CC═C(C)C═C1)N═2)C(═N3)C4═CC═C(F)C═C4)C5CCNCC5 |
| Example 2079 | [H]N3C(═C(C═2SC═C(C(═O)NCC1═NC═C(C)N═C1)N═2)C(═N3)C4═CC═C(F)C═C4)C5CCNCC5 |
| Example 2080 | [H]N3C(═C(C═2SC═C(C(═O)NCC1═CC═CC(F)═C1)N═2)C(═N3)C4═CC═C(F)C═C4)C5CCNCC5 |
| Example 2081 | [H]N3C(═C(C═2SC═C(C(═O)NCC1═CC═C(F)C═C1)N═2)C(═N3)C4═CC═C(F)C═C4)C5CCNCC5 |
| Example 2082 | [H]N3C(═C(C═2SC═C(C(═O)NCC1═CC═CC═C1F)N═2)C(═N3)C4═CC═C(F)C═C4)C5CCNCC5 |
| Example 2083 | [H]N3C(═C(C═2SC═C(C(═O)NCCCN1C═CN═C1)N═2)C(═N3)C4═CC═C(F)C═C4)C5CCNCC5 |
| Example 2084 | [H]N3C(═C(C═2SC═C(C(═O)NCCC1CCCN1C)N═2)C(═N3)C4═CC═C(F)C═C4)C5CCNCC5 |
| Example 2085 | [H]N3C(═C(C═2SC═C(C(═O)NCCN1CCCCC1)N═2)C(═N3)C4═CC═C(F)C═C4)C5CCNCC5 |
| Example 2086 | [H]N2C(═C(C═1SC═C(C(═O)NC(CCCC(C)C)C)N═1)C(═N2)C3═CC═C(F)C═C3)C4CCNCC4 |
| Example 2087 | [H]N3C(═C(C═2SC═C(C(═O)NCCN1CCOCC1)N═2)C(═N3)C4═CC═C(F)C═C4)C5CCNCC5 |
| Example 2088 | [H]N2C(═C(C═1SC═C(C(═O)NC(CC(OCC)═O)C)N═1)C(═N2)C3═CC═C(F)C═C3)C4CCNCC4 |
| Example 2089 | [H]N4C(═(C═3SC═C(C(═O)NC1CCC2═CC═CC═C12)N═3)C(═N4)C5═CC═C(F)C═C5)C6CCNCC6 |
| Example 2090 | [H]N3C(═C(C═2SC═C(C(═O)NCCC1═CC═C(C)C═C1)N═2)C(═N3)C4═CC═C(F)C═C4)C5CCNCC5 |
| Example 2091 | [H]N3C(═C(C═2SC═C(C(═O)NCC═1C(═CC═C(C)C═1)C)N═2)C(═N3)C4═CC═C(F)C═C4)C5CCNCC5 |
| Example 2092 | [H]N3C(═C(C═2SC═C(C(═O)N[C@@H](C)C1═CC═C(C)C═C1)N═2)C(═N3)C4═CC═C(F)C═C4)C5CCNCC5 |
| Example 2093 | [H]N3C(═C(C═2SC═C(C(═O)N[C@H](C)C1═CC═C(C)C═C1)N═2)C(═N3)C4═CC═C(F)C═C4)C5CCNCC5 |
| Example 2094 | [H]N3C(═C(C═2SC═C(C(═O)NCC(C)C1═CC═CC═C1)N═2)C(═N3)C4═CC═C(F)C═C4)C5CCNCC5 |
| Example 2095 | [H]N3C(═C(C═2SC═C(C(═O)NCC(C)C1═CC═CC═C1)N═2)C(═N3)C4═CC═C(F)C═C4)C5CCNCC5 |
| Example 2096 | [H]N3C(═C(C═2SC═C(C(═O)NCCCC1═CC═CC═C1)N═2)C(═N3)C4═CC═C(F)C═C4)C5CCNCC5 |
| Example 2097 | [H]N3C(═C(C═2SC═C(C(═O)NCC1═CC═C(C)C(═C1)C)N═2)C(═N3)C4═CC═C(F)C═C4)C5CCNCC5 |
| Example 2098 | [H]N3C(═C(C═2SC═C(C(═O)NC(C)CC1═CC═NC═C1)N═2)C(═N3)C4═CC═C(F)C═C4)C5CCNCC5 |
| Example 2099 | [H]N3C(═C(C═2SC═C(C(═O)NCCC1═CC═C(O)C═C1)N═2)C(═N3)C4═CC═C(F)C═C4)C5CCNCC5 |
| Example 2100 | [H]N3C(═C(C═2SC═C(C(═O)NCCOC1═CC═CC═C1)N═2)C(═N3)C4═CC═C(F)C═C4)C5CCNCC5 |
| Example 2101 | [H]N3C(═C(C═2SC═C(C(═O)NCC1═CC═CC═C1OC)N═2)C(═N3)C4═CC═C(F)C═C4)C5CCNCC5 |
| Example 2102 | [H]N3C(═C(C═2SC═C(C(═O)NCC1═CC═C(OC)C═C1)N═2)C(═N3)C4═CC═C(F)C═C4)C5CCNCC5 |
| Example 2103 | [H]N3C(═C(C═2SC═C(C(═O)NCC═1C═CC═C(OC)C═1)N═2)C(═N3)C4═CC═C(F)C═C4)C5CCNCC5 |
| Example 2104 | [H]N3C(═C(C═2SC═C(C(═O)NCCC1═CC═C(F)C═C1)N═2)C(═N3)C4═CC═C(F)C═C4)C5CCNCC5 |
| Example 2105 | [H]N3C(═C(C═2SC═C(C(═O)NCCC1═CC═CC═C1F)N═2)C(═N3)C4═CC═C(F)C═C4)C5CCNCC5 |
| Example 2106 | [H]N3C(═C(C═2SC═C(C(═O)NCCC1═CC═CC(F)═C1)N═2)C(═N3)C4═CC═C(F)C═C4)C5CCNCC5 |
| Example 2107 | [H]N3C(═C(C═2SC═C(C(═O)NCC1═CC═CC(Cl)═C1)N═2)C(═N3)C4═CC═C(F)C═C4)C5CCNCC5 |
| Example 2108 | [H]N3C(═C(C═2SC═C(C(═O)NCC1═CC═CC═C1Cl)N═2)C(═N3)C4═CC═C(F)C═C4)C5CCNCC5 |
| Example 2109 | [H]N3C(═C(C═2SC═C(C(═O)NCC1═CC═C(Cl)C═C1)N═2)C(═N3)C4═CC═C(F)C═C4)C5CCNCC5 |
| Example 2110 | [H]N3C(═C(C═2SC═C(C(═O)NCCCN1CCCC1═O)N═2)C(═N3)C4═CC═C(F)C═C4)C5CCNCC5 |
| Example 2111 | [H]N3C(═C(C═2SC═C(C(═O)NCC1═CC═C(F)C═C1F)N═2)C(═N3)C4═CC═C(F)C═C4)C5CCNCC5 |
| Example 2112 | [H]N3C(═C(C═2SC═C(C(═O)NCC1═CC═C(F)C(F)═C1)N═2)C(═N3)C4═CC═C(F)C═C4)C5CCNCC5 |
| Example 2113 | [H]N3C(═C(C═2SC═C(C(═O)NCC1═CC(F)═CC(F)═C1)N═2)C(═N3)C4═CC═C(F)C═C4)C5CCNCC5 |
| Example 2114 | [H]N3C(═C(C═2SC═C(C(═O)NCCCN1CCOCC1)N═2)C(═N3)C4═CC═C(F)C═C4)C5CCNCC5 |
| Example 2115 | [H]N2C(═C(C═1SC═C(C(═O)NCCN(C(C)C)C(C)C)N═1)C(═N2)C3═CC═C(F)C═C3)C4CCNCC4 |
| Example 2116 | [H]N4C(═C(C═3SC═C(C(═O)NC1CCCC2═CC═CC═C12)N═3)C(═N4)C5═CC═C(F)C═C5)C6CCNCC6 |
| Example 2117 | [H]N3C(═C(C═2SC═C(C(═O)NC(C)CCC1═CC═CC═C1)N═2)C(═N3)C4═CC═C(F)C═C4)C5CCNCC5 |
| Example 2118 | [H]N3C(═C(C═2SC═C(C(═O)NCC1═CC═C(C(C)C)C═C1)N═2)C(═N3)C4═CC═C(F)C═C4)C5CCNCC5 |
| Example 2119 | [H]N4C(═C(C═3SC═C(C(═O)NCC2═CC═C1OCOC1═C2)N═3)C(═N4)C5═CC═C(F)C═C5)C6CCNCC6 |
| Example 2120 | [H]N3C(═C(C═2SC═C(C(═O)NCCC═1C═CC═C(OC)C═1)N═2)C(═N3)C4═CC═C(F)C═C4)C5CCNCC5 |
| Example 2121 | [H]N3C(═C(C═2SC═C(C(═O)N[C@H](CO)CC1═CC═CC═C1)N═2)C(═N3)C4═CC═C(F)C═C4)C5CCNCC5 |
| Example 2122 | [H]N3C(═C(C═2SC═C(C(═O)NCCC1═CC═C(OC)C═C1)N═2)C(═N3)C4═CC═C(F)C═C4)C5CCNCC5 |
| Example 2123 | [H]N3C(═C(C═2SC═C(C(═O)NCCC1═CC═CC═C1OC)N═2)C(═N3)C4═CC═C(F)C═C4)C5CCNCC5 |
| Example 2124 | [H]N3C(═C(C═2SC═C(C(═O)NCC1═CC═CC═C1OCC)N═2)C(═N3)C4═CC═C═C(F)C═C4)C5CCNCC5 |
| Example 2125 | [H]N3C(═C(C═2SC═C(C(═O)NC1═CC═C(OC)C(═C1)OC)N═2)C(═N3)C4═CC═C(F)C═C4)C5CCNCC5 |
| Example 2126 | [H]N3C(═C(C═2SC═C(C(═O)NCCC1═CC═CC═C1Cl)N═2)C(═N3)C4═CC═C(F)C═C4)C5CCNCC5 |
| Example 2127 | [H]N3C(═C(C═2SC═C(C(═O)NCCC1═CC═C(Cl)C═C1)N═2)C(═N3)C4═CC═C(F)C═C4)C5CCNCC5 |
| Example 2128 | [H]N3C(═C(C═2SC═C(C(═O)NCCC1═CC═CC(Cl)═C1)N═2)C(═N3)C4═CC═C(F)C═C4)C5CCNCC5 |
| Example 2129 | [H]N3C(═C(C═2SC═C(C(═O)NC1CC(C)(C)NC(C1)(C)C)N═2)C(═N3)C4═CC═C(F)C═C4)C5CCNCC5 |
| Example 2130 | [H]N2C(═C(C═1SC═C(C(═O)NC(CCCN(CC)CC)C)N═1)C(═N2)C3═CC═C(F)C═C3)C4CCNCC4 |
| Example 2131 | [H]N3C(═C(C═2SC═C(C(═O)NCC1═CC═C(F)C(Cl)═C1)N═2)C(═N3)C4═CC═C(F)C═C4)C5CCNCC5 |
| Example 2132 | [H]N3C(═C(C═2SC═C(C(═O)N(CCC#N)CC1═CC═CC═C1)N═2)C(═N3)C4═CC═C(F)C═C4)C5CCNCC5 |
| Example 2133 | [H]N2C(═C(C═1SC═C(C(═O)NCCNC(OC(C)(C)C)═O)N═1)C(═N2)C3═CC═C(F)C═C3)C4CCNCC4 |
| Example 2134 | [H]N4C(═C(C═3SC═C(C(═O)NCCC1═CN(C2═CC═CC═C12)[H])N═3)C(═N4)C5═CC═C(F)C═C5)C6CCNCC6 |
| Example 2135 | [H]N3C(═C(C═2SC═C(C(═O)NCC1═CC═C(C(C)(C)C)C═C1)N═2)C(═N3)C4═CC═C(F)C═C4)C5CCNCC5 |
| Example 2136 | [H]N3C(═C(C═2SC═C(C(═O)NCCCN(C)C1═CC═CC═C1)N═2)C(═N3)C4═CC═C(F)C═C4)C5CCNCC5 |
| Example 2137 | [H]N3C(═C(C═2SC═C(C(═O)NCC1(O)CCCCC1)N═2)C(═N3)C4═CC═C(F)C═C4)C5CCNCC5 |
| Example 2138 | [H]N3C(═C(C═2SC═C(C(═O)NCC═1C═C(OC)C═C(C═1)OC)N═2)C(═N3)C4═CC═C(F)C═C4)C5CCNCC5 |
| Example 2139 | [H]N3C(═C(C═2SC═C(C(═O)NCC1═CC═C(OC)C(═C1)OC)N═2)C(═N3)C4═CC═C(F)C═C4)C5CCNCC5 |
| Example 2140 | [H]N3C(═C(C═2SC═C(C(═O)NCC(═O)C1═CC═CC═C1)N═2)C(═N3)C4═CC═C(F)C═C4)C5CCNCC5 |
| Example 2141 | [H]N3C(═C(C═2SC═C(C(═O)NC1CCN(C(OCC)═O)CC1)N═2)C(═N3)C4═CC═C(F)C═C4)C5CCNCC5 |
| Example 2142 | [H]N2C(═C(C═1SC═C(C(═O)NCCCNC(OC(C)(C)C)═O)N═1)C(═N2)C3═CC═C(F)C═C3)C4CCNCC4 |
| Example 2143 | [H]N3C(═C(C═2SC═C(C(═O)NCC1═CC═C(C═C1)C(F)(F)F)N═2)C(═N3)C4═CC═C(F)C═C4)C5CCNCC5 |
| Example 2144 | [H]N3C(═C(C═2SC═C(C(═O)NCC1═CC═CC(═C1)C(F)(F)F)N═2)C(═N3)C4═CC═C(F)C═C4)C5CCNCC5 |
| Example 2145 | [H]N3C(═C(C═2SC═C(C(═O)NCC1═CC═C(Cl)C(Cl)═C1)N═2)C(═N3)C4═CC═C(F)C═C4)C5CCNCC5 |
| Example 2146 | [H]N3C(═C(C═2SC═C(C(═O)NCC1═CC═C(Cl)C═C1Cl)N═2)C(═N3)C4═CC═C(F)C═C4)C5CCNCC5 |
| Example 2147 | [H]N4C(═C(C═3SC═C(C(═O)NC2CCN(CC1═CC═CC═C1)C2)N═3)C(═N4)C5═CC═C(F)C═C5)C6CNCC6 |
| Example 2148 | [H]N3C(═C(C═2SC═C(C(═O)NCCC═1C(═CC═C(OC)C═1)OC)N═2)C(═N3)C4═CC═C(F)C═C4)C5CCNCC5 |
| Example 2149 | [H]N3C(═C(C═2SC═C(C(═O)NCC1═CC═C(N(C)C)C═C1)N═2)C(═N3)C4═CC═C(F)C═C4)C5CCNCC5 |
| Example 2150 | [H]N3C(═C(C═2SC═C(C(═O)NCC1═CC═C(OC)C(═C1)O)N═2)C(═N3)C4═CC═C(F)C═C4)C5CCNCC5 |
| Example 2151 | [H]N4C(═C(C═3SC═C(C(═O)NC2CCN(CC1═CC═CC═C1)CC2)N═3)C(═N4)C5═CC═C(F)C═C5)C6CCNCC6 |
| Example 2152 | [H]N3C(═C(C═2SC═C(C(═O)NCC1═CC═CC═C1OC(F)(F)F)N═2)C(═N3)C4═CC═C(F)C═C4)C5CCNCC5 |
| Example 2153 | [H]N3C(═C(C═2SC═C(C(═O)NCCC1═CC═C(C═C1)S(═O)(═O)N)N═2)C(═N3)C4═CC═C(F)C═C4)C5CCNCC5 |
| Example 2154 | [H]N3C(═C(C═2SC═C(C(═O)NCC(C1═CC═C(OC)C═C1)═O)N═2)C(═N3)C4═CC═C(F)C═C4)C5CCNCC5 |
| Example 2155 | [H]N4C(═C(C═3SC═C(C(═O)NCCC2═CC═C1OC OC1═C2)N═3)C(═N4)C5═CC═C(F)C═C5)C6CCNCC6 |
| Example 2156 | [H]N4C(═C(C═3SC═C(C(═O)NCCC(C1═CC═CC═C1)C2═CC═CC═C2)N═3)C(═N4)C5═CC═C(F)C═C5)C6CCNCC6 |
| Example 2157 | [H]N3C(═C(C═2SC═C(C(═O)NCCNS(C1═CC═C(C)C═C1)(═O)═O)N═2)C(═N3)C4═CC═C(F)C═C4)C5CCNCC5 |

General Procedure 1: 6 μL of each acid monomer (Table 1, 0.5 M each) in DMF was transfered to a single well of a microwell plate. To these were added 4 μL of core scaffold/DIEA solution (0.5 M each) in DMF, followed by 5 μL HATU solution (0.5 M) in DMF. The reaction plate was sealed and shaken at room temperature for 16 hours, solvent was removed, and products were identified and analyzed for purity by LCMS.
General Procedure 2: 8 μL of each amine or aniline monomer (Table 2, 3, or 4, 0.5 M each, 4 mmol) in DMF was transferred to a single well of a microwell plate. To these were added 25 μL of core scaffold/DIEA solution (0.08 M core, 0.16 M DIEA) in DMF. The plate was sealed, mixed, warmed to 40° C. and left static for 16 hours. The solvent was then removed, and products were identified and analyzed for purity by LCMS.

The following compounds can generally be made using the methods described above. It is expected that these compounds when made will have activity similar to those that have been made in the examples above.

CC(C)NC(═O)c1nc(cs1)n2c(nnc2c3ccc(F)cn3)C4CCOCC4

CC(C)NC(═O)n1ccc(n1)n2c(nnc2c3ccc(F)cc3)C4CCOCC4

COc1cc(F)ccc1c2nnc(C3CCOCC3)n2c4nc(cs4)C(═O)NC(C)C

CC(C)CNC(═O)n1ccc(n1)n2c(nnc2c3ccc(F)cn3)C4CCOCC4

COc1cc(F)ccc1c2nnc(C3CCOCC3)n2c4csc(n4)C(═O)NCC(C)C

OCC(CO)CNC(═O)c1csc(n1)n2c(nnc2c3ccc(F)cn3)C4CCOCC4

OCC(CO)CNC(═O)c1nc(cs1)n2c(nnc2c3ccc(F)cc3)C4CCOCC4

COc1cc(F)ccc1c2nnc(C3CCOCC3)n2c4ccn(n4)C(═O)NCC(CO)CO

CC(C)NC(═O)n1ccc(n1)n2c(nnc2c3ccc(F)cn3)C4CCNCC4

CC(C)NC(═O)c1csc(n1)n2c(nnc2c3ccc(F)cn3)C4CCNCC4

CC(C)NC(═O)c1nc(cs1)n2c(nnc2c3ccc(F)cc3)C4CCNCC4

COc1cc(F)ccc1c2nnc(C3CCNCC3)n2c4ccn(n4)C(═O)NC(C)C

CC(C)CNC(═O)n1ccc(n1)n2c(nnc2c3ccc(F)cc3)C4CCNCC4

OCC(CO)CNC(═O)c1nc(cs1)n2c(nnc2c3ccc(F)cn3)C4CCNCC4

OCC(CO)CNC(═O)c1csc(n1)n2c(nnc2c3ccc(F)cc3)C4CCNCC4

COCC(═O)N1CCC(CC1)c2nnc(c3ccc(F)cn3)n2c4ccn(n4)C(═O)NC(C)C

COCC(═O)N1CCC(CC1)c2nnc(c3ccc(F)cn3)n2c4nc(cs4)C(═O)NC(C)C

COCC(═O)N1CCC(CC1)c2nnc(c3ccc(F)cc3)n2c4csc(n4)C(═O)NC(C)C

COCC(═O)N1CCC(CC1)c2nnc(c3ccc(F)cc3OC)n2c4ccn(n4)C(═O)NC(C)C

COCC(═O)N1CCC(CC1)c2nnc(c3ccc(F)cn3)n2c4csc(n4)C(═O)NCC(C)C

COCC(═O)N1CCC(CC1)c2nnc(c3ccc(F)cc3)n2c4nc(cs4)C(═O)NCC(C)C

COCC(═O)N1CCC(CC1)c2nnc(c3ccc(F)cc3)n2c4ccn(n4)C(═O)NCC(CO)CO

CC(C)NC(═O)n1ccc(n1)n2c(OCCCN3CCOCC3)nnc2c4ccc(F)cn4

CC(C)NC(═O)c1csc(n1)n2c(OCCCN3CCOCC3)nnc2c4ccc(F)cn4

CC(C)NC(═O)c1nc(cs1)n2c(OCCCN3CCOCC3)nnc2c4ccc(F)cc4

COc1cc(F)ccc1c2nnc(OCCCN3CCOCC3)n2c4nc(cs4)C(═O)NC(C)C

OCC(CO)CNC(═O)n1ccc(n1)n2c(OCCCN3CCOCC3)nnc2c4ccc(F)cc4

CC(C)NC(═O)c1nc(cs1)n2c(OCCCN3CCNCC3)nnc2c4ccc(F)cn4

CC(C)NC(═O)c1csc(n1)n2c(OCCCN3CCNCC3)nnc2c4ccc(F)cc4

COc1cc(F)ccc1c2nnc(OCCCN3CCNCC3)n2c4ccn(n4)C(═O)NC(C)C

CC(C)CNC(═O)c1csc(n1)n2c(OCCCN3CCNCC3)nnc2c4ccc(F)cn4

COc1cc(F)ccc1c2nnc(OCCCN3CCNCC3)n2c4csc(n4)C(═O)NCC(C)C

COc1cc(F)ccc1c2nnc(OCCCN3CCNCC3)n2c4nc(cs4)C(═O)NCC(C)C

OCC(CO)CNC(═O)n1ccc(n1)n2c(OCCCN3CCNCC3)nnc2c4ccc(F)cn4

CC(C)NC(═O)c1csc(n1)n2c(NCCN3CCOCC3)nnc2c4ccc(F)cn4

CC(C)NC(═O)c1nc(cs1)n2c(NCCN3CCOCC3)nnc2c4ccc(F)cc4

COc1cc(F)ccc1c2nnc(NCCN3CCOCC3)n2c4ccn(n4)C(═O)NCC(C)C

OCC(CO)CNC(═O)c1csc(n1)n2c(NCCN3CCOCC3)nnc2c4ccc(F)cc4

CC(C)NC(═O)n1ccc(n1)n2c(NCCN3CCNCC3)nnc2c4ccc(F)cn4

COc1cc(F)ccc1c2nnc(NCCN3CCNCC3)n2c4ccn(n4)C(═O)NC(C)C

COc1cc(F)ccc1c2nnc(NCCN3CCNCC3)n2c4nc(cs4)C(═O)NC(C)C

CC(C)CNC(═O)c1nc(cs1)n2c(NCCN3CCNCC3)nnc2c4ccc(F)cn4

COc1cc(F)ccc1c2nnc(NCCN3CCNCC3)n2c4csc(n4)C(═O)NCC(C)C

OCC(CO)CNC(═O)c1csc(n1)n2c(NCCN3CCNCC3)nnc2c4ccc(F)cn4

OCC(CO)CNC(═O)n1ccc(n1)n2c(NCCN3CCNCC3)nnc2c4ccc(F)cc4

CC(C)NC(═O)c1nc(cs1)n2c(NCCN3CCN(CC3)C(═O)CO)nnc2c4ccc(F)cn4

CC(C)NC(═O)n1ccc(n1)n2c(NCCN3CCN(CC3)C(═O)CO)nnc2c4ccc(F)cc4

CC(C)NC(═O)c1csc(n1)n2c(NCCN3CCN(CC3)C(═O)CO)nnc2c4ccc(F)cc4

CC(C)CNC(═O)n1ccc(n1)n2c(NCCN3CCN(CC3)C(═O)CO)nnc2c4ccc(F)cn4

CC(C)NC(═O)c1nc(cs1)n2c(CCCN3CCOCC3)nnc2c4ccc(F)cn4

CC(C)NC(═O)n1ccc(n1)n2c(CCCN3CCOCC3)nnc2c4ccc(F)cc4

CC(C)NC(═O)c1csc(n1)n2c(CCCN3CCOCC3)nnc2c4ccc(F)cc4

CC(C)CNC(═O)c1csc(n1)n2c(CCCN3CCOCC3)nnc2c4ccc(F)cn4

CC(C)CNC(═O)c1nc(cs1)n2c(CCCN3CCOCC3)nnc2c4ccc(F)cc4

OCC(CO)CNC(═O)n1ccc(n1)n2c(CCCN3CCOCC3)nnc2c4ccc(F)cn4

CC(C)NC(═O)n1ccc(n1)n2c(CCCN3CCNCC3)nnc2c4ccc(F)cn4

COc1cc(F)ccc1c2nnc(CCCN3CCNCC3)n2c4csc(n4)C(═O)NC(C)C

COc1cc(F)ccc1c2nnc(CCCN3CCNCC3)n2c4ccn(n4)C(═O)NCC(C)C

COc1cc(F)ccc1c2nnc(CCCN3CCNCC3)n2c4nc(cs4)C(═O)NCC(C)C

OCC(CO)CNC(═O)c1nc(cs1)n2c(CCCN3CCNCC3)nnc2c4ccc(F)cn4

CC(C)NC(═O)n1ccc(n1)n2c(CCN3CCOCC3)nnc2c4ccc(F)cn4

CC(C)NC(═O)c1nc(cs1)n2c(CCN3CCOCC3)nnc2c4ccc(F)cc4

COc1cc(F)ccc1c2nnc(CCN3CCOCC3)n2c4ccn(n4)C(═O)NC(C)C

COc1cc(F)ccc1c2nnc(CCN3CCOCC3)n2c4nc(cs4)C(═O)NC(C)C

CC(C)CNC(═O)c1nc(cs1)n2c(CCN3CCOCC3)nnc2c4ccc(F)cn4

COc1cc(F)ccc1c2nnc(CCN3CCOCC3)n2c4csc(n4)C(═O)NCC(C)C

OCC(CO)CNC(═O)n1ccc(n1)n2c(CCN3CCOCC3)nnc2c4ccc(F)cc4

OCC(CO)CNC(═O)c1csc(n1)n2c(CCN3CCOCC3)nnc2c4ccc(F)cc4

CC(C)NC(═O)c1csc(n1)n2c(CCN3CCNCC3)nnc2c4ccc(F)cn4

CC(C)NC(═O)n1ccc(n1)n2c(CCN3CCNCC3)nnc2c4ccc(F)cc4

CC(C)CNC(═O)n1ccc(n1)n2c(CCN3CCNCC3)nnc2c4ccc(F)cn4

CC(C)CNC(═O)c1csc(n1)n2c(CCN3CCNCC3)nnc2c4ccc(F)cc4

OCC(CO)CNC(═O)c1nc(cs1)n2c(CCN3CCNCC3)nnc2c4ccc(F)cc4

CNC(═O)N1CCN(CCc2nnc(c3ccc(F)cn3)n2c4csc(n4)C(═O)NC(C)C)CC1

CNC(═O)N1CCN(CCc2nnc(c3ccc(F)cc3)n2c4ccn(n4)C(═O)NC(C)C)CC1

CNC(═O)N1CCN(CCc2nnc(c3ccc(F)cc3)n2c4nc(cs4)C(═O)NC(C)C)CC1

CNC(═O)N1CCN(CCc2nnc(c3ccc(F)cn3)n2c4ccn(n4)C(═O)NCC(C)C)CC1

CNC(═O)N1CCN(CCc2nnc(c3ccc(F)cn3)n2c4nc(cs4)C(═O)NCC(C)C)CC1

CNC(═O)N1CCN(CCc2nnc(c3ccc(F)cc3)n2c4csc(n4)C(═O)NCC(C)C)CC1

CC(C)NC(═O)c1csc(n1)n2c(CN3CCOCC3)nnc2c4ccc(F)cn4

COc1cc(F)ccc1c2nnc(CN3CCOCC3)n2c4csc(n4)C(═O)NC(C)C

CC(C)CNC(═O)n1ccc(n1)n2c(CN3CCOCC3)nnc2c4ccc(F)cn4

COc1cc(F)ccc1c2nnc(CN3CCOCC3)n2c4ccn(n4)C(═O)NCC(C)C

COc1cc(F)ccc1c2nnc(CN3CCOCC3)n2c4nc(cs4)C(═O)NCC(C)C

OCC(CO)CNC(═O)c1nc(cs1)n2c(CN3CCOCC3)nnc2c4ccc(F)cn4

OCC(CO)CNC(═O)c1nc(cs1)n2c(CN3CCOCC3)nnc2c4ccc(F)cc4

CC(C)NC(═O)n1ccc(n1)n2c(CN3CCNCC3)nnc2c4ccc(F)cn4

CC(C)NC(═O)c1nc(cs1)n2c(CN3CCNCC3)nnc2c4ccc(F)cn4

CC(C)NC(═O)c1nc(cs1)n2c(CN3CCNCC3)nnc2c4ccc(F)cc4

CC(C)NC(═O)c1csc(n1)n2c(CN3CCNCC3)nnc2c4ccc(F)cc4

COc1cc(F)ccc1c2nnc(CN3CCNCC3)n2c4ccn(n4)C(═O)NC(C)C

CC(C)CNC(═O)c1csc(n1)n2c(CN3CCNCC3)nnc2c4ccc(F)cn4

CC(C)CNC(═O)n1ccc(n1)n2c(CN3CCNCC3)nnc2c4ccc(F)cc4

COc1cc(F)ccc1c2nnc(CN3CCNCC3)n2c4csc(n4)C(═O)NCC(C)C

CN═C(S)N1CCN(Cc2nnc(c3ccc(F)cn3)n2c4ccn(n4)C(═O)NC(C)C)CC1

CN═C(S)N1CCN(Cc2nnc(c3ccc(F)cn3)n2c4csc(n4)C(═O)NC(C)C)CC1

CN═C(S)N1CCN(Cc2nnc(c3ccc(F)cc3)n2c4ccn(n4)C(═O)NC(C)C)CC1

CN═C(S)N1CCN(Cc2nnc(c3ccc(F)cc3)n2c4nc(cs4)C(═O)NC(C)C)CC1

CNC(═S)N1CCN(Cc2nnc(c3ccc(F)cc3OC)n2c4ccn(n4)C(═O)NC(C)C)CC1

CN═C(S)N1CCN(Cc2nnc(c3ccc(F)cn3)n2c4nc(cs4)C(═O)NCC(C)C)CC1

CN═C(S)N1CCN(Cc2nnc(c3ccc(F)cc3)n2c4csc(n4)C(═O)NCC(C)C)CC1

CC(C)NC(═O)c1nc(cs1)n2c(nnc2c3ccc(F)cn3)N4CCOCC4

CC(C)NC(═O)n1ccc(n1)n2c(nnc2c3ccc(F)cc3)N4CCOCC4

CC(C)NC(═O)c1csc(n1)n2c(nnc2c3ccc(F)cc3)N4CCOCC4

COc1cc(F)ccc1c2nnc(N3CCOCC3)n2c4csc(n4)C(═O)NC(C)C

CC(C)CNC(═O)n1ccc(n1)n2c(nnc2c3ccc(F)cn3)N4CCOCC4

CC(C)CNC(═O)c1csc(n1)n2c(nnc2c3ccc(F)cn3)N4CCOCC4

COc1cc(F)ccc1c2nnc(N3CCOCC3)n2c4ccn(n4)C(═O)NCC(C)C

COc1cc(F)ccc1c2nnc(N3CCOCC3)n2c4nc(cs4)C(═O)NCC(C)C

OCC(CO)CNC(═O)c1nc(cs1)n2c(nnc2c3ccc(F)cc3)N4CCOCC4

CC(C)NC(═O)n1ccc(n1)n2c(nnc2c3ccc(F)cn3)N4CCNCC4

CC(C)NC(═O)c1csc(n1)n2c(nnc2c3ccc(F)cn3)N4CCNCC4

CC(C)NC(═O)c1nc(cs1)n2c(nnc2c3ccc(F)cc3)N4CCNCC4

CC(C)CNC(═O)c1nc(cs1)n2c(nnc2c3ccc(F)cn3)N4CCNCC4

CC(C)CNC(═O)n1ccc(n1)n2c(nnc2c3ccc(F)cc3)N4CCNCC4

CC(C)CNC(═O)c1csc(n1)n2c(nnc2c3ccc(F)cc3)N4CCNCC4

COc1cc(F)ccc1c2nnc(N3CCNCC3)n2c4csc(n4)C(═O)NCC(CO)CO

CC(C)NC(═O)c1nc(cs1)n2c(nnc2c3ccc(F)cc3)N4CCN(CC4)C(═O)CO

COc1cc(F)ccc1c2nnc(N3 CCN(CC3)C(═O)CO)n2c4ccn(n4)C(═O)NC(C)C

COc1cc(F)ccc1c2nnc(N3CCN(CC3)C(═O)CO)n2c4nc(cs4)C(═O)NC(C)C

CC(C)CNC(═O)c1csc(n1)n2c(nnc2c3ccc(F)cc3)N4CCN(CC4)C(═O)CO

COc1cc(F)ccc1c2nnc(N3CCN(CC3)C(═O)CO)n2c4csc(n4)C(═O)NCC(C)C

OCC(CO)CNC(═O)n1ccc(n1)n2c(nnc2c3ccc(F)cn3)N4CCN(CC4)C(═O)CO

OCC(CO)CNC(═O)n1ccc(n1)n2c(nnc2c3ccc(F)cc3)N4CCN(CC4)C(═O)CO

CCN1CN(CCc2nnc(c3ccc(F)cn3)n2c4csc(n4)C(═O)NC(C)C)C1

CCN1CN(CCc2nnc(c3ccc(F)cc3)n2c4nc(cs4)C(═O)NC(C)C)C1

CCN1CN(CCc2nnc(c3ccc(F)cc3OC)n2c4csc(n4)C(═O)NC(C)C)C1

CCN1CN(CCc2nnc(c3ccc(F)cc3)n2c4ccn(n4)C(═O)NCC(C)C)C1

CCN1CN(CCc2nnc(c3ccc(F)cc3)n2c4csc(n4)C(═O)NCC(C)C)C1

CCN1CN(CCc2nnc(c3ccc(F)cc3OC)n2c4nc(cs4)C(═O)NCC(C)C)C1

CCN1CN(CCc2nnc(c3ccc(F)cn3)n2c4nc(cs4)C(═O)NCC(CO)CO)C1

CC(C)NC(═O)c1csc(n1)n2c(nnc2c3ccc(F)cc3)C4CCNC4

COc1cc(F)ccc1c2nnc(C3CCNC3)n2c4csc(n4)C(═O)NC(C)C

CC(C)CNC(═O)c1nc(cs1)n2c(nnc2c3ccc(F)cc3)C4CCNC4

COc1cc(F)ccc1c2nnc(C3CCNC3)n2c4nc(cs4)C(═O)NCC(C)C

OCC(CO)CNC(═O)n1ccc(n1)n2c(nnc2c3ccc(F)cn3)C4CCNC4

COCC(═O)N1CCN(CC1)c2nnc(c3ccc(F)cn3)n2c4ccn(n4)C(═O)NC(C)C

COCC(═O)N1CCN(CC1)c2nnc(c3ccc(F)cn3)n2c4nc(cs4)C(═O)NC(C)C

COCC(═O)N1CCN(CC1)c2nnc(c3ccc(F)cc3)n2c4nc(cs4)C(═O)NC(C)C

COCC(═O)N1CCN(CC1)c2nnc(c3ccc(F)cn3)n2c4csc(n4)C(═O)NCC(C)C

CC(C)NC(═O)n1ccc(n1)n2c(SCCCN(C)C)nnc2c3ccc(F)cc3

COc1cc(F)ccc1c2nnc(SCCCN(C)C)n2c3csc(n3)C(═O)NC(C)C

CC(C)CNC(═O)c1nc(cs1)n2c(SCCCN(C)C)nnc2c3ccc(F)cc3

CC(C)CNC(═O)c1csc(n1)n2c(SCCCN(C)C)nnc2c3ccc(F)cc3

CN(C)CCCSc1nnc(c2ccc(F)cn2)n1c3csc(n3)C(═O)NCC(CO)CO

COc1cc(F)ccc1c2nnc(SCCCN(C)C)n2c3 ccn(n3)C(═O)NCC(CO)CO

COCCCc1nnc(c2ccc(F)cn2)n1c3ccn(n3)C(═O)NC(C)C

COCCCc1nnc(c2ccc(F)cn2)n1c3nc(cs3)C(═O)NC(C)C

COCCCc1nnc(c2ccc(F)cc2)n1c3csc(n3)C(═O)NC(C)C

COCCCc1nnc(c2ccc(F)cc2OC)n1c3ccn(n3)C(═O)NC(C)C

COCCCc1nnc(c2ccc(F)cn2)n1c3csc(n3)C(═O)NCC(C)C

COCCCc1nnc(c2ccc(F)cc2)n1c3ccn(n3)C(═O)NCC(C)C

COCCCc1nnc(c2ccc(F)cc2)n1c3nc(cs3)C(═O)NCC(C)C

COCCCc1nnc(c2ccc(F)cc2)n1c3csc(n3)C(═O)NCC(CO)CO

COc1cc(F)ccc1c2nnc(N(C)C)n2c3 ccn(n3)C(═O)NC(C)C

COc1cc(F)ccc1c2nnc(N(C)C)n2c3nc(cs3)C(═O)NC(C)C

CN(C)c1nnc(c2ccc(F)cn2)n1c3ccn(n3)C(═O)NCC(CO)CO

CN(C)c1nnc(c2ccc(F)cc2)n1c3ccn(n3)C(═O)NCC(CO)CO

CN(C)c1nnc(c2ccc(F)cc2)n1c3nc(cs3)C(═O)NCC(CO)CO

CC(C)NC(═O)n1ccc(n1)n2cnnc2c3ccc(F)cn3

CC(C)NC(═O)c1csc(n1)n2cnnc2c3ccc(F)cn3

CC(C)NC(═O)n1ccc(n1)n2cnnc2c3ccc(F)cc3

CC(C)NC(═O)c1nc(cs1)n2cnnc2c3ccc(F)cc3

CC(C)CNC(═O)c1nc(cs1)n2cnnc2c3ccc(F)cn3

CC(C)CNC(═O)c1csc(n1)n2cnnc2c3ccc(F)cc3

COc1cc(F)ccc1c2nncn2c3 ccn(n3)C(═O)NCC(C)C

COc1cc(F)ccc1c2nncn2c3csc(n3)C(═O)NCC(C)C

OCC(CO)CNC(═O)n1ccc(n1)n2cnnc2c3ccc(F)cc3

OCC(CO)CNC(═O)c1nc(cs1)n2cnnc2c3ccc(F)cc3

COc1cc(F)ccc1c2nncn2c3nc(cs3)C(═O)NCC(CO)CO

CC(C)NC(═O)c1ncn(n1)C2=C(C(═O)NN2C3CCOCC3)c4ccc(F)cn4

CC(C)NC(═O)c1ccn(n1)C2=C(C(═O)ON2C3CCOCC3)c4ccc(F)cc4

CC(C)NC(═O)c1c[nH]c(n1)n2c(nnc2c3ccc(F)cc3)C4CCNCC4

CC(C)CNC(═O)c1cnn(n1)C2=C(C(═O)ON2C3CCNCC3)c4ccc(F)cn4

COc1cc(F)ccc1C2=C(N(NC2=O)C3CCNCC3)c4oc(cc4)C(═O)NCC(CO)CO

COCC(═O)N1CCC(CC1)N2NC(═O)C(═C2c3 ccn(n3)C(═O)NC(C)C)c4ccc(F)cc4

COCC(═O)N1CCC(CC1)N2OC(═O)C(═C2c3c[nH]c(n3)C(═O)NC(C)C)c4ccc(F)cc4OC

COCC(═O)N1CCC(CC1)c2nnc(c3ccc(F)cn3)n2c4occ(n4)C(═O)NCC(C)C

CC(C)NC(═O)c1oc(cc1)n2c(OCCCN3CCOCC3)nnc2c4ccc(F)cc4

CC(C)NC(═O)c1occ(n1)n2c(NCCN3CCNCC3)nnc2c4ccc(F)cn4

CC(C)CNC(═O)c1c[nH]c(n1)n2c(NCCN3CCN(CC3)C(═O)CO)nnc2c4ccc(F)cc4

OCC(CO)CNC(═O)c1ncn(n1)C2=C(C(═O)NN2CCCN3CCOCC3)c4ccc(F)cc4

CC(C)CNC(═O)n1ccc(n1)C2=C(C(═O)ON2CCN3CCOCC3)c4ccc(F)cc4

COc1cc(F)ccc1c2nnc(CCN3CCNCC3)n2c4c[nH]c(n4)C(═O)NCC(CO)CO

CNC(═O)N1CCN(CCN2NC(═O)C(═C2n3ncc(n3)C(═O)NC(C)C)c4ccc(F)cc4)CC1

CNC(═O)N1CCN(CCc2nnc(c3ccc(F)cc3OC)n2c4occ(n4)C(═O)NC(C)C)CC1

CNC(═O)N1CCN(CCN2OC(═O)C(═C2c3 nc(c[n H]3)C(═O)NCC(C)C)c4ccc(F)cn4)CC1

CC(C)NC(═O)c1nc(c[nH]1)C2=C(C(═O)NN2CN3CCNCC3)c4ccc(F)cc4

COc1cc(F)ccc1C2=C(N(CN3CCNCC3)SC2=O)n4cnc(n4)C(═O)NCC(C)C

OCC(CO)CNC(═O)n1ccc(n1)C2=C(C(═O)NN2CN3CCNCC3)c4ccc(F)cn4

CN═C(S)N1CCN(CN2OC(═O)C(═C2c3oc(cn3)C(═O)NC(C)C)c4ccc(F)cn4)CC1

CN═C(S)N1CCN(Cc2nnc(c3ccc(F)cc3)n2c4coc(n4)C(═O)NCC(C)C)CC1

COc1cc(F)ccc1C2=C(N(NC2=O)N3 CCOCC3)c4occ(n4)C(═O)NC(C)C

CC(C)CNC(═O)c1ncn(n1)C2=C(C(═O)ON2N3CCOCC3)c4ccc(F)cn4

OCC(CO)CNC(═O)c1nc(c[nH]1)C2=C(C(═O)NN2N3CCOCC3)c4ccc(F)cn4

CC(C)NC(═O)c1cnn(n1)C2=C(C(═O)NN2N3CCNCC3)c4ccc(F)cn4

CC(C)NC(═O)c1oc(cc1)C2=C(C(═O)SN2N3CCNCC3)c4ccc(F)cc4

CC(C)CNC(═O)c1ccn(n1)C2=C(C(═O)NN2N3CCNCC3)c4ccc(F)cc4

OCC(CO)CNC(═O)c1coc(n1)n2c(nnc2c3ccc(F)cc3)N4CCNCC4

CC(C)NC(═O)n1ccc(n1)C2=C(C(═O)ON2N3CCN(CC3)C(═O)CO)c4ccc(F)cn4

CC(C)NC(═O)c1nc(c[nH]1)n2c(nnc2c3ccc(F)cn3)N4CCN(CC4)C(═O)CO

COc1cc(F)ccc1C2=C(N(OC2=O)N3CCN(CC3)C(═O)CO)n4ncc(n4)C(═O)NCC(C)C

CCN1CN(CCN2NC(═O)C(═C2c3oc(nc3)C(═O)NCC(C)C)c4ccc(F)cc4)C1

CCN1CN(CCN2OC(═O)C(═C2n3ccc(n3)C(═O)NCC(CO)CO)c4ccc(F)cn4)C1

COCC(═O)N1CCN(CC1)N2NC(═O)C(═C2n3cnc(n3)C(═O)NC(C)C)c4ccc(F)cc4

COCC(═O)N1CCN(CC1)c2nnc(c3ccc(F)cc3OC)n2c4coc(n4)C(═O)NC(C)C

CN(C)CCCSc1nnc(c2ccc(F)cn2)n1c3nc(c[nH]3)C(═O)NCC(CO)CO

COCCCN1SC(═O)C(═C1c2oc(cn2)C(═O)NCC(CO)CO)c3ccc(F)cc3

CC(C)NC(═O)c1c[nH]c(n1)c2n[nH]c(O)c2c3ccc(F)cn3

CC(C)NC(═O)c1oc(cc1)n2cnnc2c3ccc(F)cn3

CC(C)NC(═O)c1nc(c[nH]1)c2nsc(O)c2c3ccc(F)cc3

CC(C)NC(═O)c1ccn(n1)c2nsc(O)c2c3ccc(F)cc3

COc1cc(F)ccc1c2c(O)[nH]nc2c3 ccn(n3)C(═O)NC(C)C

CC(C)CNC(═O)c1cnn(n1)c2nsc(O)c2c3ccc(F)cn3

CC(C)CNC(═O)c1ncn(n1)c2n[nH]c(O)c2c3ccc(F)cc3

COc1cc(F)ccc1c2c(O)snc2c3csc(n3)C(═O)NCC(C)C

OCC(CO)CNC(═O)n1ccc(n1)c2nsc(O)c2c3ccc(F)cn3

OCC(CO)CNC(═O)c1coc(n1)c2n[nH]c(O)c2c3ccc(F)cc3

CC(C)NC(═O)c1oc(cc1)C2=C(C(═O)NN2C)c3ccc(F)cn3

CC(C)CNC(═O)c1oc(cc1)n2c(CO)nnc2c3ccc(F)cc3

CC(C)NC(═O)n1ccc(n1)n2nc(nc2c3ccc(F)cn3)C4CCOCC4

CC(C)NC(═O)n1ccc(n1)c2oc(nc2c3ccc(F)cc3)C4CCOCC4

COc1cc(F)ccc1c2nc(nn2c3nc(cs3)C(═O)NC(C)C)C4CCOCC4

OCC(CO)CNC(═O)c1nc(cs1)c2oc(nc2c3ccc(F)cn3)C4CCOCC4

OCC(CO)CNC(═O)c1csc(n1)c2oc(nc2c3ccc(F)cc3)C4CCOCC4

COc1cc(F)ccc1c2nc(nn2c3csc(n3)C(═O)NCC(CO)CO)C4CCOCC4

COc1cc(F)ccc1c2nc(oc2n3ccc(n3)C(═O)NCC(CO)CO)C4CCOCC4

CC(C)NC(═O)c1nc(cs1)n2nc(nc2c3ccc(F)cn3)C4CCNCC4

CC(C)NC(═O)c1csc(n1)c2oc(nc2c3ccc(F)cn3)C4CCNCC4

CC(C)NC(═O)c1nc(cs1)c2oc(nc2c3ccc(F)cc3)C4CCNCC4

CC(C)CNC(═O)c1ccn(n1)c2oc(nc2c3ccc(F)cn3)C4CCNCC4

CC(C)CNC(═O)c1nc(cs1)n2nc(nc2c3ccc(F)cc3)C4CCNCC4

COc1cc(F)ccc1c2nc(oc2c3 ccn(n3)C(═O)NCC(C)C)C4CCNCC4

OCC(CO)CNC(═O)c1csc(n1)n2nc(nc2c3ccc(F)cn3)C4CCNCC4

OCC(CO)CNC(═O)n1ccc(n1)n2nc(nc2c3ccc(F)cc3)C4CCNCC4

COc1cc(F)ccc1c2nc(oc2c3csc(n3)C(═O)NCC(CO)CO)C4CCNCC4

COCC(═O)N1CCC(CC1)c2nc(c3ccc(F)cn3)n(n2)c4nc(cs4)C(═O)NC(C)C

COCC(═O)N1CCC(CC1)c2oc(c3csc(n3)C(═O)NC(C)C)c(n2)c4ccc(F)cn4

COCC(═O)N1CCC(CC1)c2oc(c(n2)c3ccc(F)cc3)n4ccc(n4)C(═O)NC(C)C

COCC(═O)N1CCC(CC1)c2nc(c3ccc(F)cc3OC)n(n2)c4csc(n4)C(═O)NC(C)C

COCC(═O)N1CCC(CC1)c2oc(c3nc(cs3)C(═O)NC(C)C)c(n2)c4ccc(F)cc4OC

COCC(═O)N1CCC(CC1)c2nc(c3ccc(F)cn3)n(n2)c4csc(n4)C(═O)NCC(C)C

COCC(═O)N1CCC(CC1)c2oc(c3nc(cs3)C(═O)NCC(C)C)c(n2)c4ccc(F)cn4

COCC(═O)N1CCC(CC1)c2oc(c3csc(n3)C(═O)NCC(C)C)c(n2)c4ccc(F)cc4

COCC(═O)N1CCC(CC1)c2nc(c3ccc(F)cc3OC)n(n2)c4ccn(n4)C(═O)NCC(C)C

COCC(═O)N1CCC(CC1)c2oc(c3 ccn(n3)C(═O)NCC(CO)CO)c(n2)c4ccc(F)cn4

COCC(═O)N1CCC(CC1)c2oc(c3ccc(F)cn3)c(n2)n4ccc(n4)C(═O)NCC(CO)CO

CC(C)NC(═O)n1ccc(n1)n2nc(OCCCN3CCOCC3)nc2c4ccc(F)cn4

CC(C)NC(═O)c1nc(cs1)c2nc(OCCCN3CCOCC3)oc2c4ccc(F)cc4

COc1cc(F)ccc1c2nc(OCCCN3CCOCC3)oc2n4 ccc(n4)C(═O)NC(C)C

CC(C)CNC(═O)c1csc(n1)c2oc(OCCCN3CCOCC3)nc2c4ccc(F)cn4

CC(C)CNC(═O)c1csc(n1)n2nc(OCCCN3CCOCC3)nc2c4ccc(F)cc4

CC(C)NC(═O)c1nc(cs1)n2nc(OCCCN3CCNCC3)nc2c4ccc(F)cn4

CC(C)NC(═O)c1csc(n1)c2nc(OCCCN3CCNCC3)oc2c4ccc(F)cc4

COc1cc(F)ccc1c2oc(OCCCN3CCNCC3)nc2c4ccn(n4)C(═O)NC(C)C

CC(C)CNC(═O)c1nc(cs1)c2oc(OCCCN3CCNCC3)nc2c4ccc(F)cn4

OCC(CO)CNC(═O)n1ccc(n1)c2oc(OCCCN3CCNCC3)nc2c4ccc(F)cn4

OCC(CO)CNC(═O)c1ccn(n1)c2nc(OCCCN3CCNCC3)oc2c4ccc(F)cn4

OCC(CO)CNC(═O)n1ccc(n1)n2nc(OCCCN3CCNCC3)nc2c4ccc(F)cc4

CC(C)NC(═O)c1nc(cs1)n2nc(NCCN3CCOCC3)nc2c4ccc(F)cc4

COc1cc(F)ccc1c2oc(NCCN3CCOCC3)nc2c4nc(cs4)C(═O)NC(C)C

CC(C)CNC(═O)c1nc(cs1)c2oc(NCCN3CCOCC3)nc2c4ccc(F)cn4

COc1cc(F)ccc1c2nc(NCCN3CCOCC3)nn2c4nc(cs4)C(═O)NCC(C)C

OCC(CO)CNC(═O)n1ccc(n1)n2nc(NCCN3CCOCC3)nc2c4ccc(F)cn4

OCC(CO)CNC(═O)n1ccc(n1)c2nc(NCCN3CCOCC3)oc2c4ccc(F)cn4

OCC(CO)CNC(═O)c1ccn(n1)c2nc(NCCN3CCOCC3)oc2c4ccc(F)cc4

CC(C)NC(═O)c1csc(n1)n2nc(NCCN3CCNCC3)nc2c4ccc(F)cn4

CC(C)NC(═O)n1ccc(n1)c2nc(NCCN3CCNCC3)oc2c4ccc(F)cn4

CC(C)NC(═O)c1nc(cs1)c2oc(NCCN3CCNCC3)nc2c4ccc(F)cc4

COc1cc(F)ccc1c2nc(NCCN3CCNCC3)nn2c4ccn(n4)C(═O)NC(C)C

COc1cc(F)ccc1c2nc(NCCN3CCNCC3)nn2c4csc(n4)C(═O)NCC(C)C

COc1cc(F)ccc1c2oc(NCCN3CCNCC3)nc2n4ccc(n4)C(═O)NCC(C)C

OCC(CO)CNC(═O)c1nc(cs1)n2nc(NCCN3CCNCC3)nc2c4ccc(F)cn4

OCC(CO)CNC(═O)c1csc(n1)c2oc(NCCN3CCNCC3)nc2c4ccc(F)cn4

OCC(CO)CNC(═O)c1csc(n1)n2nc(NCCN3CCNCC3)nc2c4ccc(F)cc4

COc1cc(F)ccc1c2nc(NCCN3CCNCC3)oc2c4ccn(n4)C(═O)NCC(CO)CO

CC(C)NC(═O)c1nc(cs1)n2nc(NCCN3CCN(CC3)C(═O)CO)nc2c4ccc(F)cn4

CC(C)NC(═O)c1ccn(n1)c2oc(NCCN3CCN(CC3)C(═O)CO)nc2c4ccc(F)cn4

CC(C)NC(═O)c1csc(n1)c2nc(NCCN3CCN(CC3)C(═O)CO)oc2c4ccc(F)cn4

CC(C)NC(═O)c1csc(n1)n2nc(NCCN3CCN(CC3)C(═O)CO)nc2c4ccc(F)cc4

CC(C)CNC(═O)n1ccc(n1)n2nc(NCCN3 CCN(CC3)C(═O)CO)nc2c4ccc(F)cc4

CC(C)CNC(═O)n1ccc(n1)c2nc(NCCN3CCN(CC3)C(═O)CO)oc2c4ccc(F)cc4

CC(C)NC(═O)n1ccc(n1)n2nc(CCCN3CCOCC3)nc2c4ccc(F)cc4

CC(C)NC(═O)c1csc(n1)c2nc(CCCN3CCOCC3)oc2c4ccc(F)cc4

CC(C)CNC(═O)n1ccc(n1)c2oc(CCCN3CCOCC3)nc2c4ccc(F)cn4

CC(C)CNC(═O)c1ccn(n1)c2oc(CCCN3CCOCC3)nc2c4ccc(F)cc4

COc1cc(F)ccc1c2nc(CCCN3CCOCC3)nn2c4csc(n4)C(═O)NCC(C)C

COc1cc(F)ccc1c2oc(CCCN3CCOCC3)nc2c4ccn(n4)C(═O)NCC(CO)CO

CC(C)NC(═O)c1nc(cs1)c2oc(CCCN3CCNCC3)nc2c4ccc(F)cn4

CC(C)NC(═O)c1csc(n1)n2nc(CCCN3CCNCC3)nc2c4ccc(F)cc4

CC(C)CNC(═O)n1ccc(n1)n2nc(CCCN3CCNCC3)nc2c4ccc(F)cn4

COc1cc(F)ccc1c2nc(CCCN3CCNCC3)oc2c4nc(cs4)C(═O)NCC(C)C

OCC(CO)CNC(═O)c1nc(cs1)c2nc(CCCN3CCNCC3)oc2c4ccc(F)cc4

CC(C)NC(═O)n1ccc(n1)c2nc(CCN3CCOCC3)oc2c4ccc(F)cc4

CC(C)CNC(═O)c1csc(n1)n2nc(CCN3CCOCC3)nc2c4ccc(F)cn4

COc1cc(F)ccc1c2nc(CCN3CCOCC3)oc2c4csc(n4)C(═O)NCC(C)C

COc1cc(F)ccc1c2nc(CCN3CCOCC3)nn2c4ccn(n4)C(═O)NCC(CO)CO

CC(C)NC(═O)c1ccn(n1)c2nc(CCN3CCNCC3)oc2c4ccc(F)cn4

CC(C)CNC(═O)n1ccc(n1)c2oc(CCN3CCNCC3)nc2c4ccc(F)cc4

OCC(CO)CNC(═O)c1nc(cs1)n2nc(CCN3CCNCC3)nc2c4ccc(F)cc4

OCC(CO)CNC(═O)c1ccn(n1)c2nc(CCN3CCNCC3)oc2c4ccc(F)cc4

OCC(CO)CNC(═O)c1csc(n1)c2nc(CCN3CCNCC3)oc2c4ccc(F)cc4

CNC(═O)N1CCN(CCc2nc(c3ccc(F)cn3)n(n2)c4csc(n4)C(═O)NC(C)C)CC1

CNC(═O)N1CCN(CCc2oc(c3nc(cs3)C(═O)NC(C)C)c(n2)c4ccc(F)cn4)CC1

CNC(═O)N1CCN(CCc2oc(c3csc(n3)C(═O)NC(C)C)c(n2)c4ccc(F)cc4)CC1

CNC(═O)N1CCN(CCc2oc(c3ccc(F)cc3)c(n2)n4ccc(n4)C(═O)NC(C)C)CC1

CNC(═O)N1CCN(CCc2oc(c3 ccn(n3)C(═O)NC(C)C)c(n2)c4ccc(F)cc4OC)CC1

CNC(═O)N1CCN(CCc2oc(c3 csc(n3)C(═O)NCC(C)C)c(n2)c4ccc(F)cn4)CC1

CNC(═O)N1CCN(CCc2nc(c3ccc(F)cc3)n(n2)c4ccn(n4)C(═O)NCC(C)C)CC1

CNC(═O)N1CCN(CCc2nc(c3ccc(F)cc3)n(n2)c4nc(cs4)C(═O)NCC(C)C)CC1

CNC(═O)N1CCN(CCc2oc(c3nc(cs3)C(═O)NCC(C)C)c(n2)c4ccc(F)cc4)CC1

CC(C)NC(═O)n1ccc(n1)c2oc(CN3CCOCC3)nc2c4ccc(F)cn4

CC(C)NC(═O)c1nc(cs1)c2oc(CN3CCOCC3)nc2c4ccc(F)cn4

CC(C)CNC(═O)c1nc(cs1)n2nc(CN3CCOCC3)nc2c4ccc(F)cn4

CC(C)CNC(═O)c1csc(n1)c2nc(CN3CCOCC3)oc2c4ccc(F)cc4

COc1cc(F)ccc1c2oc(CN3CCOCC3)nc2n4ccc(n4)C(═O)NCC(C)C

OCC(CO)CNC(═O)c1ccn(n1)c2oc(CN3CCOCC3)nc2c4ccc(F)cn4

OCC(CO)CNC(═O)c1csc(n1)n2nc(CN3CCOCC3)nc2c4ccc(F)cc4

OCC(CO)CNC(═O)n1ccc(n1)c2oc(CN3CCOCC3)nc2c4ccc(F)cc4

OCC(CO)CNC(═O)c1nc(cs1)c2oc(CN3CCOCC3)nc2c4ccc(F)cc4

CC(C)NC(═O)n1ccc(n1)c2oc(CN3CCNCC3)nc2c4ccc(F)cc4

CC(C)NC(═O)c1ccn(n1)c2nc(CN3CCNCC3)oc2c4ccc(F)cc4

COc1cc(F)ccc1c2nc(CN3CCNCC3)nn2c4csc(n4)C(═O)NC(C)C

COc1cc(F)ccc1c2nc(CN3CCNCC3)oc2c4csc(n4)C(═O)NC(C)C

CC(C)CNC(═O)n1ccc(n1)n2nc(CN3CCNCC3)nc2c4ccc(F)cc4

OCC(CO)CNC(═O)c1nc(cs1)c2oc(CN3CCNCC3)nc2c4ccc(F)cn4

CN═C(S)N1CCN(Cc2nc(c3ccc(F)cn3)n(n2)c4ccn(n4)C(═O)NC(C)C)CC1

CN═C(S)N1CCN(Cc2oc(c(n2)c3ccc(F)cn3)n4ccc(n4)C(═O)NC(C)C)CC1

CN═C(S)N1CCN(Cc2nc(c3ccc(F)cc3)n(n2)c4nc(cs4)C(═O)NC(C)C)CC1

CN═C(S)N1CCN(Cc2oc(c3csc(n3)C(═O)NC(C)C)c(n2)c4ccc(F)cc4)CC1

CNC(═S)N1CCN(Cc2nc(c3ccc(F)cc3OC)n(n2)c4ccn(n4)C(═O)NC(C)C)CC1

CN═C(S)N1CCN(Cc2oc(c3 ccn(n3)C(═O)NCC(C)C)c(n2)c4ccc(F)cn4)CC1

CN═C(S)N1CCN(Cc2oc(c3ccc(F)cn3)c(n2)c4nc(cs4)C(═O)NCC(C)C)CC1

CN═C(S)N1CCN(Cc2nc(c3ccc(F)cc3)n(n2)c4csc(n4)C(═O)NCC(C)C)CC1

CC(C)NC(═O)c1csc(n1)n2nc(nc2c3ccc(F)cn3)N4CCOCC4

CC(C)NC(═O)c1ccn(n1)c2oc(nc2c3ccc(F)cn3)N4CCOCC4

CC(C)NC(═O)c1nc(cs1)n2nc(nc2c3ccc(F)cc3)N4CCOCC4

CC(C)NC(═O)c1csc(n1)c2oc(nc2c3ccc(F)cc3)N4CCOCC4

COc1cc(F)ccc1c2nc(nn2c3 ccn(n3)C(═O)NC(C)C)N4CCOCC4

CC(C)CNC(═O)n1ccc(n1)n2nc(nc2c3ccc(F)cn3)N4CCOCC4

CC(C)CNC(═O)n1ccc(n1)c2oc(nc2c3ccc(F)cc3)N4CCOCC4

CC(C)CNC(═O)c1nc(cs1)c2oc(nc2c3ccc(F)cc3)N4CCOCC4

OCC(CO)CNC(═O)c1nc(cs1)n2nc(nc2c3ccc(F)cn3)N4CCOCC4

OCC(CO)CNC(═O)c1csc(n1)c2oc(nc2c3ccc(F)cn3)N4CCOCC4

OCC(CO)CNC(═O)c1ccn(n1)c2oc(nc2c3ccc(F)cc3)N4CCOCC4

COc1cc(F)ccc1c2nc(nn2c3nc(cs3)C(═O)NCC(CO)CO)N4CCOCC4

CC(C)NC(═O)n1ccc(n1)c2oc(nc2c3ccc(F)cn3)N4CCNCC4

CC(C)NC(═O)c1nc(cs1)c2oc(nc2c3ccc(F)cn3)N4CCNCC4

CC(C)NC(═O)c1csc(n1)n2nc(nc2c3ccc(F)cc3)N4CCNCC4

CC(C)NC(═O)c1ccn(n1)c2oc(nc2c3ccc(F)cc3)N4CCNCC4

COc1cc(F)ccc1c2nc(nn2c3csc(n3)C(═O)NCC(C)C)N4CCNCC4

COc1cc(F)ccc1c2oc(nc2c3nc(cs3)C(═O)NCC(C)C)N4CCNCC4

OCC(CO)CNC(═O)c1csc(n1)n2nc(nc2c3ccc(F)cn3)N4CCNCC4

COc1cc(F)ccc1c2nc(oc2c3ccn(n3)C(═O)NCC(CO)CO)N4CCNCC4

COc1cc(F)ccc1c2nc(oc2c3ccn(n3)C(═O)NC(C)C)N4CCN(CC4)C(═O)CO

COc1cc(F)ccc1c2nc(oc2c3csc(n3)C(═O)NC(C)C)N4CCN(CC4)C(═O)CO

COc1cc(F)ccc1c2oc(nc2n3ccc(n3)C(═O)NC(C)C)N4CCN(CC4)C(═O)CO

CC(C)CNC(═O)c1nc(cs1)c2nc(oc2c3ccc(F)cn3)N4CCN(CC4)C(═O)CO

CC(C)CNC(═O)n1ccc(n1)n2nc(nc2c3ccc(F)cc3)N4CCN(CC4)C(═O)CO

CC(C)CNC(═O)c1csc(n1)n2nc(nc2c3ccc(F)cc3)N4CCN(CC4)C(═O)CO

OCC(CO)CNC(═O)c1ccn(n1)c2oc(nc2c3ccc(F)cn3)N4CCN(CC4)C(═O)CO

OCC(CO)CNC(═O)n1ccc(n1)c2oc(nc2c3ccc(F)cn3)N4CCN(CC4)C(═O)CO

CCN1CN(CCc2nc(c3ccc(F)cn3)n(n2)c4ccn(n4)C(═O)NC(C)C)C1

CCN1CN(CCc2nc(c3ccc(F)cc3)n(n2)c4csc(n4)C(═O)NC(C)C)C1

CCN1CN(CCc2oc(c3ccc(F)cc3OC)c(n2)n4ccc(n4)C(═O)NC(C)C)C1

CCN1CN(CCc2oc(c3ccc(F)cc3OC)c(n2)c4nc(cs4)C(═O)NC(C)C)C1

CCN1CN(CCc2oc(c3nc(cs3)C(═O)NCC(C)C)c(n2)c4ccc(F)cn4)C1

CCN1CN(CCc2nc(c3ccc(F)cc3OC)n(n2)c4nc(cs4)C(═O)NCC(C)C)C1

CCN1CN(CCc2nc(c3ccc(F)cn3)n(n2)c4csc(n4)C(═O)NCC(CO)CO)C1

CCN1CN(CCc2oc(c3 ccn(n3)C(═O)NCC(CO)CO)c(n2)c4ccc(F)cn4)C1

CC(C)NC(═O)n1ccc(n1)c2oc(nc2c3ccc(F)cn3)C4CCNC4

CC(C)NC(═O)c1ccn(n1)c2nc(oc2c3ccc(F)cc3)C4CCNC4

COc1cc(F)ccc1c2nc(nn2c3 ccn(n3)C(═O)NC(C)C)C4CCNC4

CC(C)CNC(═O)c1csc(n1)n2nc(nc2c3ccc(F)cc3)C4CCNC4

COCC(═O)N1CCN(CC1)c2nc(c3ccc(F)cn3)n(n2)c4ccn(n4)C(═O)NC(C)C

COCC(═O)N1CCN(CC1)c2nc(c3ccc(F)cn3)n(n2)c4csc(n4)C(═O)NC(C)C

COCC(═O)N1CCN(CC1)c2oc(c3nc(cs3)C(═O)NC(C)C)c(n2)c4ccc(F)cn4

COCC(═O)N1CCN(CC1)c2nc(c3ccc(F)cc3OC)n(n2)c4nc(cs4)C(═O)NC(C)C

COCC(═O)N1CCN(CC1)c2nc(c3ccc(F)cc3)n(n2)c4csc(n4)C(═O)NCC(C)C

COCC(═O)N1CCN(CC1)c2oc(c3nc(cs3)C(═O)NCC(C)C)c(n2)c4ccc(F)cc4

CC(C)NC(═O)c1csc(n1)n2nc(SCCCN(C)C)nc2c3ccc(F)cn3

CC(C)NC(═O)n1ccc(n1)c2nc(oc2c3ccc(F)cn3)SCCCN(C)C

COc1cc(F)ccc1c2nc(SCCCN(C)C)nn2c3csc(n3)C(═O)NC(C)C

COc1cc(F)ccc1c2nc(SCCCN(C)C)nn2c3 ccn(n3)C(═O)NCC(C)C

CN(C)CCCSc1oc(c2ccc(F)cc2)c(n1)c3csc(n3)C(═O)NCC(CO)CO

COCCCc1nc(c2ccc(F)cc2)n(n1)c3csc(n3)C(═O)NC(C)C

COCCCc1oc(c2csc(n2)C(═O)NC(C)C)c(n1)c3ccc(F)cc3OC

COCCCc1oc(c2ccc(F)cc2OC)c(n1)c3 ccn(n3)C(═O)NC(C)C

COCCCc1oc(c2csc(n2)C(═O)NCC(C)C)c(n1)c3ccc(F)cn3

COCCCc1oc(c2nc(cs2)C(═O)NCC(CO)CO)c(n1)c3ccc(F)cn3

COCCCc1nc(c2ccc(F)cc2OC)n(n1)c3nc(cs3)C(═O)NCC(CO)CO

CC(C)NC(═O)n1ccc(n1)c2oc(nc2c3ccc(F)cc3)N(C)C

CC(C)NC(═O)c1nc(cs1)c2oc(nc2c3ccc(F)cc3)N(C)C

CN(C)c1oc(c2ccc(F)cn2)c(n1)c3csc(n3)C(═O)NCC(CO)CO

CN(C)c1nc(c2ccc(F)cc2)n(n1)c3csc(n3)C(═O)NCC(CO)CO

CN(C)c1oc(c2nc(cs2)C(═O)NCC(CO)CO)c(n1)c3ccc(F)cc3

COc1cc(F)ccc1c2nc(nn2c3 ccn(n3)C(═O)NCC(CO)CO)N(C)C

CC(C)NC(═O)c1nc(cs1)c2ocnc2c3ccc(F)cn3

CC(C)NC(═O)c1csc(n1)c2ncoc2c3ccc(F)cn3

CC(C)NC(═O)n1ccc(n1)n2ncnc2c3ccc(F)cc3

CC(C)NC(═O)c1csc(n1)n2ncnc2c3ccc(F)cc3

CC(C)NC(═O)c1ccn(n1)c2ncoc2c3ccc(F)cc3

COc1cc(F)ccc1c2ncnn2c3csc(n3)C(═O)NC(C)C

CC(C)CNC(═O)c1nc(cs1)n2ncnc2c3ccc(F)cn3

CC(C)CNC(═O)c1csc(n1)n2ncnc2c3ccc(F)cn3

CC(C)CNC(═O)n1ccc(n1)c2ocnc2c3ccc(F)cn3

CC(C)CNC(═O)c1csc(n1)c2ocnc2c3ccc(F)cc3

COc1cc(F)ccc1c2ncnn2c3 ccn(n3)C(═O)NCC(C)C

COc1cc(F)ccc1c2ncnn2c3nc(cs3)C(═O)NCC(C)C

COc1cc(F)ccc1c2ocnc2n3ccc(n3)C(═O)NCC(C)C

OCC(CO)CNC(═O)n1ccc(n1)n2ncnc2c3ccc(F)cn3

OCC(CO)CNC(═O)c1ccn(n1)c2ocnc2c3ccc(F)cn3

OCC(CO)CNC(═O)n1ccc(n1)c2ncoc2c3ccc(F)cc3

CC(C)NC(═O)c1csc(n1)c2nc(C)oc2c3ccc(F)cc3

CC(C)CNC(═O)c1ccn(n1)c2nc(C)oc2c3ccc(F)cc3

COc1cc(F)ccc1c2nc(C)oc2c3nc(cs3)C(═O)NCC(CO)CO

CC(C)NC(═O)c1csc(n1)n2nc(CO)nc2c3ccc(F)cn3

CC(C)NC(═O)c1ccn(n1)c2nc(CO)oc2c3ccc(F)cn3

CC(C)CNC(═O)c1nc(cs1)c2nc(CO)oc2c3ccc(F)cn3

OCC(CO)CNC(═O)c1nc(cs1)c2oc(CO)nc2c3ccc(F)cc3

CC(C)NC(═O)c1ncn(n1)c2nn(C3CCOCC3)C(O)c2c4ccc(F)cn4

CC(C)NC(═O)c1ncn(n1)C2=C(ON(C3CCNCC3)C2=O)c4ccc(F)cc4

COc1cc(F)ccc1c2nc(nn2c3nc(c[nH]3)C(═O)NC(C)C)C4CCNCC4

OCC(CO)CNC(═O)c1coc(n1)c2oc(nc2c3ccc(F)cn3)C4CCNCC4

COc1cc(F)ccc1c2c(O)n(nc2n3ccc(n3)C(═O)NCC(CO)CO)C4CCNCC4

COc1cc(F)ccc1C2=C(ON(C3CCNCC3)C2=O)c4c[nH]c(n4)C(═O)NCC(CO)CO

COCC(═O)N1CCC(CC1)N2OC(═C(C2=O)c3ccc(F)cn3)n4ncc(n4)C(═O)NC(C)C

COCC(═O)N1CCC(CC1)c2oc(c3coc(n3)C(═O)NC(C)C)c(n2)c4ccc(F)cc4

COCC(═O)N1CCC(CC1)c2nc(c3ccc(F)cn3)n(n2)c4c[n H]c(n4)C(═O)NCC(C)C

COCC(═O)N1CCC(CC1)n2nc(c3oc(cc3)C(═O)NCC(C)C)c(c20)c4ccc(F)cc4

OCC(CO)CNC(═O)c1c[nH]c(n1)n2nc(OCCCN3CCOCC3)nc2c4ccc(F)cn4

CC(C)NC(═O)c1nc(c[nH]1)c2oc(OCCCN3CCN CC3)nc2c4ccc(F)cc4

CC(C)CNC(═O)c1oc(cn1)c2oc(NCCN3CCOCC3)nc2c4ccc(F)cn4

CC(C)CNC(═O)c1coc(n1)n2nc(NCCN3CCN(CC3)C(═O)CO)nc2c4ccc(F)cc4

CC(C)NC(═O)n1ccc(n1)C2=C(ON(CCCN3CCOCC3)C2=O)c4ccc(F)cc4

COc1cc(F)ccc1c2c(O)n(CCCN3CCNCC3)nc2c4ccn(n4)C(═O)NCC(C)C

COc1cc(F)ccc1c2nc(CCN3CCOCC3)oc2n4cnc(n4)C(═O)NCC(CO)CO

OCC(CO)CNC(═O)c1cnn(n1)C2=C(ON(CCN3CCNCC3)C2=O)c4ccc(F)cc4

CNC(═O)N1CCN(CCc2oc(c3nc(c[nH]3)C(═O)NC(C)C)c(n2)c4ccc(F)cn4)CC1

CNC(═O)N1CCN(CCN2OC(═C(C2=O)c3ccc(F)cc3)n4cnc(n4)C(═O)NCC(C)C)CC1

CC(C)NC(═O)c1occ(n1)n2nc(CN3CCOCC3)nc2c4ccc(F)cc4

CC(C)CNC(═O)c1cnn(n1)c2nn(CN3CCOCC3)C(O)c2c4ccc(F)cc4

CC(C)NC(═O)c1oc(cn1)C2=C(ON(CN3CCNCC3)C2=O)c4ccc(F)cn4

COc1cc(F)ccc1c2nc(CN3CCNCC3)oc2n4ncc(n4)C(═O)NC(C)C

CN═C(S)N1CCN(Cn2nc(c3 ccn(n3)C(═O)NC(C)C)c(c20)c4ccc(F)cn4)CC1

COc1cc(F)ccc1c2oc(nc2c3oc(cc3)C(═O)NC(C)C)N4CCOCC4

CC(C)CNC(═O)c1ccn(n1)C2=C(ON(N3CCOCC3)C2=O)c4ccc(F)cn4

OCC(CO)CNC(═O)c1oc(cn1)c2nn(N3CCOCC3)C(O)c2c4ccc(F)cc4

CC(C)NC(═O)c1coc(n1)n2nc(nc2c3ccc(F)cn3)N4CCNCC4

OCC(CO)CNC(═O)c1ncn(n1)C2=C(C(═O)N(O2)N3CCNCC3)c4ccc(F)cn4

OCC(CO)CNC(═O)c1c[nH]c(n1)c2nc(oc2c3ccc(F)cc3)N4CCNCC4

CC(C)NC(═O)c1cnn(n1)c2nn(N3CCN(CC3)C(═O)CO)C(O)c2c4ccc(F)cn4

CC(C)NC(═O)c1ncn(n1)c2oc(nc2c3ccc(F)cc3)N4CCN(CC4)C(═O)CO

COc1cc(F)ccc1C2=C(C(═O)N(O2)N3CCN(CC3)C(═O)CO)c4occ(n4)C(═O)NC(C)C

CCN1CN(CCN2OC(═C(C2=O)c3ccc(F)cc3)c4nc(c[nH]4)C(═O)NC(C)C)C1

CCN1CN(CCc2oc(c3ccc(F)cc3)c(n2)c4occ(n4)C(═O)NC(C)C)C1

CCN1CN(CCn2nc(c3c[nH]c(n3)C(═O)NCC(CO)CO)c(c20)c4ccc(F)cn4)C1

COCC(═O)N1CCN(CC1)n2nc(c3c[nH]c(n3)C(═O)NC(C)C)c(c20)c4ccc(F)cc4OC

COCC(═O)N1CCN(CC1)c2nc(c3ccc(F)cc3)n(n2)c4oc(cc4)C(═O)NCC(CO)CO

COCCCn1nc(c2nc(c[nH]2)C(═O)NC(C)C)c(c1O)c3ccc(F)cc3

COCCCN1SC(═C(C I═O)c2 ccc(F)cc2)n3ccc(n3)C(═O)NCC(CO)CO

CC(C)NC(═O)c1nc(c[nH]1)n2nc(nc2c3ccc(F)cc3)N(C)C

CC(C)NC(═O)c1nc(c[nH]1)C2=C(ONC2=O)c3ccc(F)cn3

CC(C)NC(═O)c1ccn(n1)c2n[nH]c(O)c2c3ccc(F)cc3

CC(C)NC(═O)c1ccn(n1)C2=C(C(═O)NO2)c3ccc(F)cc3

COc1cc(F)ccc1c2c(O)[nH]nc2c3oc(cn3)C(═O)NCC(C)C

COc1cc(F)ccc1C2=C(ONC2=O)c3oc(cc3)C(═O)NCC(C)C

OCC(CO)CNC(═O)n1ccc(n1)c2n[nH]c(O)c2c3ccc(F)cn3

OCC(CO)CNC(═O)n1ccc(n1)C2=C(C(═O)NO2)c3ccc(F)cn3

OCC(CO)CNC(═O)c1occ(n1)n2ncnc2c3ccc(F)cn3

COCC(═O)N1CCC(CC1)c2nnc(c3ccc(F)cc3OC)n2c4nc(cs4)C(═O)NCC(C)C

COCC(═O)N1CCC(CC1)c2nnc(c3ccc(F)cn3)n2c4nc(cs4)C(═O)NCC(CO)CO

COCC(═O)N1CCC(CC1)c2nnc(c3ccc(F)cc3OC)n2c4csc(n4)C(═O)NCC(CO)CO

CC(C)CNC(═O)c1nc(cs1)n2c(OCCCN3CCOCC3)nnc2c4ccc(F)cc4

OCC(CO)CN C(═O)c1csc(n1)n2c(OCCCN3CCOCC3)nnc2c4ccc(F)cn4

COc1cc(F)ccc1c2nnc(OCCCN3CCOCC3)n2c4ccn(n4)C(═O)NCC(CO)CO

OCC(CO)CNC(═O)c1nc(cs1)n2c(OCCCN3CCNCC3)nnc2c4ccc(F)cn4

COCC(═O)N1CCN(CCCOc2nnc(c3ccc(F)cn3)n2c4ccn(n4)C(═O)NC(C)C)CC1

COCC(═O)N1CCN(CCCOc2nnc(c3ccc(F)cc3OC)n2c4nc(cs4)C(═O)NC(C)C)CC1

COc1cc(F)ccc1c2nnc(NCCN3CCNCC3)n2c4nc(cs4)C(═O)NCC(CO)CO

COc1cc(F)ccc1c2nnc(NCCN3CCN(CC3)C(═O)CO)n2c4ccn(n4)C(═O)NC(C)C

CC(C)CNC(═O)c1csc(n1)n2c(NCCN3CCN(CC3)C(═O)CO)nnc2c4ccc(F)cn4

OCC(CO)CNC(═O)n1ccc(n1)n2c(NCCN3CCN(CC3)C(═O)CO)nnc2c4ccc(F)cn4

OCC(CO)CNC(═O)c1nc(cs1)n2c(NCCN3CCN(CC3)C(═O)CO)nnc2c4ccc(F)cc4

CC(C)NC(═O)c1csc(n1)n2c(CCCN3CCN(CC3)C(═O)OC(C)C)nnc2c4ccc(F)cc4

COc1cc(F)ccc1c2nnc(CCCN3CCN(CC3)C(═O)OC(C)C)n2c4ccn(n4)C(═O)NC(C)C

CC(C)CNC(═O)c1nc(cs1)n2c(CCCN3CCN(CC3)C(═O)OC(C)C)nnc2c4ccc(F)cn4

CNC(═O)N1CCN(CCc2nnc(c3ccc(F)cc3OC)n2c4ccn(n4)C(═O)NCC(C)C)CC1

CNC(═O)N1CCN(CCc2nnc(c3ccc(F)cc3OC)n2c4nc(cs4)C(═O)NCC(C)C)CC1

CNC(═O)N1CCN(CCc2nnc(c3ccc(F)cn3)n2c4ccn(n4)C(═O)NCC(CO)CO)CC1

CNC(═O)N1CCN(CCc2nnc(c3ccc(F)cc3)n2c4csc(n4)C(═O)NCC(CO)CO)CC1

CNC(═S)N1CCN(Cc2nnc(c3ccc(F)cc3OC)n2c4csc(n4)C(═O)NC(C)C)CC1

CN═C(S)N1CCN(Cc2nnc(c3ccc(F)cn3)n2c4nc(cs4)C(═O)NCC(CO)CO)CC1

CN═C(S)N1CCN(Cc2nnc(c3ccc(F)cc3)n2c4ccn(n4)C(═O)NCC(CO)CO)CC1

OCC(CO)CNC(═O)c1nc(cs1)n2c(nnc2c3ccc(F)cn3)N4CCN(CC4)C(═O)CO

OCC(CO)CNC(═O)c1csc(n1)n2c(nnc2c3ccc(F)cc3)N4CCN(CC4)C(═O)CO

CCN1CN(CCc2nnc(c3ccc(F)cc3OC)n2c4csc(n4)C(═O)NCC(CO)CO)C1

COCC(═O)N1CCN(CC1)c2nnc(c3ccc(F)cc3OC)n2c4csc(n4)C(═O)NCC(C)C

COCC(═O)N1CCN(CC1)c2nnc(c3ccc(F)cc3OC)n2c4ccn(n4)C(═O)NCC(CO)CO

COc1cc(F)ccc1c2nnc(SCCCN(C)C)n2c3nc(cs3)C(═O)NCC(CO)CO

COCC(═O)N1CCC(CC1)c2nc(c3ccc(F)cc3OC)n(n2)c4nc(cs4)C(═O)NCC(C)C

COCC(═O)N1CCC(CC1)c2oc(c3nc(cs3)C(═O)NCC(CO)CO)c(n2)c4ccc(F)cc4

CC(C)CNC(═O)n1ccc(n1)c2oc(OCCCN3CCOCC3)nc2c4ccc(F)cc4

OCC(CO)CNC(═O)c1csc(n1)c2oc(OCCCN3CCOCC3)nc2c4ccc(F)cn4

COCC(═O)N1CCN(CCCOc2oc(c3 csc(n3)C(═O)NC(C)C)c(n2)c4ccc(F)cn4)CC1

COCC(═O)N1CCN(CCCOc2nc(c3ccc(F)cn3)n(n2)c4ccn(n4)C(═O)NCC(C)C)CC1

COCC(═O)N1CCN(CCCOc2nc(c3ccc(F)cc3)n(n2)c4csc(n4)C(═O)NCC(CO)CO)CC1

CC(C)CNC(═O)c1nc(cs1)c2oc(NCCN3 CCNCC3)nc2c4ccc(F)cc4

COc1cc(F)ccc1c2nc(NCCN3CCNCC3)nn2c4nc(cs4)C(═O)NCC(CO)CO

COc1cc(F)ccc1c2nc(NCCN3CCN(CC3)C(═O)CO)nn2c4csc(n4)C(═O)NC(C)C

OCC(CO)CNC(═O)c1ccn(n1)c2oc(NCCN3CCN(CC3)C(═O)CO)nc2c4ccc(F)cn4

OCC(CO)CNC(═O)n1ccc(n1)c2oc(NCCN3CCN(CC3)C(═O)CO)nc2c4ccc(F)cc4

CC(C)NC(═O)n1ccc(n1)c2oc(CCCN3CCN(CC3)C(═O)OC(C)C)nc2c4ccc(F)cn4

CC(C)NC(═O)c1csc(n1)n2nc(CCCN3CCN(CC3)C(═O)OC(C)C)nc2c4ccc(F)cc4

CC(C)CNC(═O)c1nc(cs1)n2nc(CCCN3CCN(CC3)C(═O)OC(C)C)nc2c4ccc(F)cn4

COc1cc(F)ccc1c2nc(CCCN3CCN(CC3)C(═O)OC(C)C)oc2n4 ccc(n4)C(═O)NCC(CO)CO

CNC(═O)N1CCN(CCc2oc(c3nc(cs3)C(═O)NC(C)C)c(n2)c4ccc(F)cc4OC)CC1

CNC(═O)N1CCN(CCc2nc(c3ccc(F)cn3)n(n2)c4nc(cs4)C(═O)NCC(CO)CO)CC1

CNC(═S)N1CCN(Cc2oc(c3 ccn(n3)C(═O)NCC(C)C)c(n2)c4ccc(F)cc4OC)CC1

CN═C(S)N1CCN(Cc2oc(c3csc(n3)C(═O)NCC(CO)CO)c(n2)c4ccc(F)cn4)CC1

CN═C(S)N1CCN(Cc2nc(c3ccc(F)cc3)n(n2)c4ccn(n4)C(═O)NCC(CO)CO)CC1

OCC(CO)CNC(═O)c1nc(cs1)n2nc(nc2c3ccc(F)cn3)N4CCN(CC4)C(═O)CO

OCC(CO)CNC(═O)c1nc(cs1)c2nc(oc2c3ccc(F)cc3)N4CCN(CC4)C(═O)CO

CCN1CN(CCc2oc(c3csc(n3)C(═O)NC(C)C)c(n2)c4ccc(F)cc4)C1

CCN1CN(CCc2nc(c3ccc(F)cc3)n(n2)c4csc(n4)C(═O)NCC(C)C)C1

COCC(═O)N1CCN(CC1)c2oc(c3ccc(F)cc3OC)c(n2)c4ccn(n4)C(═O)NCC(CO)CO

CC(C)NC(═O)c1ccn(n1)c2oc(SCCCN(C)C)nc2c3ccc(F)cc3

CC(C)CNC(═O)c1csc(n1)n2nc(SCCCN(C)C)nc2c3ccc(F)cc3

COc1cc(F)ccc1c2oc(SCCCN(C)C)nc2c3csc(n3)C(═O)NCC(CO)CO

CC(C)CNC(═O)c1csc(n1)c2oc(nc2c3ccc(F)cc3)N(C)C

### Biological Activity Assay

Assays

The activity of the compounds in examples 1-2196 has been shown to be p38 inhibitors by using the following assays. The other compounds listed above, which have not yet been made, are predicted to have activity in these assays as well.

p38α Biochemical Assay

The p38α biochemical assay employed is based on measurement of total ATP turnover following enzyme incubation with substrate in the presence of ATP with the use of a luminescent detection reagent (Cambrex PKlight). The assays were performed in 1536-well white opaque plates.

The final volume was 7.5005 μL as prepared prepared from the addition of 5 ul of kinase reaction (p38 alpah+MapkapK2+ATP) with 0.0005 μL compound dissolved in DMSO, and 2.5 ul of the detection reagent. Assay buffer contains the following reagents to give final concentration in the assay: 200 mM Tris, 100 mM MgCl2, 1.5 mM EGTA, 4 mM CaCl2, 20 mM MOPS, 1 mM EDTA, 1% glycerol, 0.1% B-Mecaptoethanol, and 1 mg/ml BSA. Test compounds are pinned using proprietary pintool technology (Kalypsys, Inc) and delivered as 40 nl amounts into the 5 ul mixture of active p38 alpha enzyme (Upstate Biotechnology) and MapkapK2 (Upstate Biotechnology) whole protein as a substrate for phosphorylation in the presence of 1.4 uM final concentration ATP. Reactions are incubated at 30C for 2 hours and detection reagent is added in 2.5 ul/well amounts. Assay is read using a Perkin Elmer Viewlux. Data is represented as IC50 in uM as determined by GraphPad Prism (GraphPad Software, Inc) as shown in Table 1 below.

Results

IC₅₀ data were obtained for the compounds provided herein. Most of the compounds exhibited p38α kinase IC₅₀ values of less than 10 μM, many less than 1 μM. Data for selected compounds is shown in the Table 1 below.

In the p38 inhibitor assay compounds of the invention generally have IC₅₀ values of around 30 μM and below. The more active compounds have IC₅₀ values of around 500 nM and below. The compounds of the invention are clearly potent inhibitors of p38 kinase, especially p38α kinase. In Table 1 below, (+) indicates that a compound had an IC₅₀ of ≦1 μM, whereas a (−) indicates that a compound had an IC₅₀ of >1 μM (but were not necessarily inactive).

TABLE 1
Biological Activity

| Example | IC₅₀ |
|---|---|
| 1 | + |
| 2 | + |
| 3 | − |
| 4 | + |
| 5 | + |
| 6 | + |
| 7 | − |
| 8 | + |
| 9 | + |
| 10 | − |
| 11 | − |
| 12 | − |
| 13 | − |
| 14 | − |
| 15 | + |
| 16 | + |
| 17 | + |
| 18 | + |
| 19 | − |
| 20 | − |
| 21 | + |
| 22 | + |
| 23 | + |
| 24 | − |
| 25 | + |
| 26 | + |
| 27 | + |
| 28 | + |
| 29 | + |
| 30 | − |
| 31 | − |
| 32 | − |
| 33 | − |
| 34 | + |
| 35 | + |
| 36 | + |
| 37 | + |
| 38 | + |
| 39 | − |
| 40 | ND |
| 41 | + |
| 42 | + |
| 43 | − |
| 44 | + |
| 46 | + |
| 47 | + |
| 48 | + |
| 49 | + |
| 50 | + |
| 51 | + |
| 52 | + |
| 53 | + |
| 54 | + |
| 55 | + |
| 56 | + |
| 57 | + |
| 58 | − |
| 59 | + |
| 60 | − |
| 61 | + |
| 62 | + |
| 63 | + |
| 64 | + |
| 65 | + |
| 66 | + |
| 67 | + |
| 68 | + |
| 69 | + |
| 70 | − |
| 71 | + |
| 72 | + |
| 73 | + |
| 74 | + |
| 75 | + |
| 76 | + |
| 77 | + |
| 78 | + |
| 79 | + |
| 80 | + |
| 81 | + |
| 82 | + |
| 83 | + |
| 84 | + |
| 85 | + |
| 86 | + |
| 87 | + |
| 88 | − |
| 89 | − |
| 90 | − |
| 91 | + |
| 92 | + |
| 93 | + |
| 94 | + |
| 95 | − |
| 96 | + |
| 97 | − |
| 98 | − |
| 99 | − |
| 100 | + |
| 101 | + |
| 102 | + |
| 103 | + |
| 104 | + |
| 105 | + |
| 106 | + |
| 107 | − |
| 108 | + |
| 109 | + |
| 110 | + |
| 111 | − |
| 112 | + |
| 113 | + |
| 114 | − |
| 115 | + |
| 116 | + |
| 117 | − |
| 118 | + |
| 119 | + |
| 120 | + |
| 121 | − |
| 122 | − |
| 123 | − |
| 124 | + |
| 125 | − |
| 126 | − |
| 127 | − |
| 128 | + |
| 129 | + |
| 130 | + |
| 131 | + |
| 132 | − |
| 133 | + |
| 134 | − |
| 135 | − |
| 136 | − |
| 137 | − |
| 138 | − |
| 139 | − |
| 140 | − |
| 141 | − |
| 142 | − |
| 143 | + |
| 144 | + |
| 145 | − |
| 146 | + |
| 147 | + |
| 148 | + |
| 149 | − |
| 150 | + |
| 151 | + |
| 152 | + |
| 153 | + |
| 154 | − |
| 155 | − |
| 156 | + |
| 157 | + |
| 158 | + |
| 159 | + |
| 160 | − |
| 161 | − |
| 162 | − |
| 163 | − |
| 164 | − |
| 165 | − |
| 166 | + |
| 167 | + |
| 168 | + |
| 169 | − |
| 170 | − |
| 171 | − |
| 172 | − |
| 173 | − |
| 174 | − |
| 175 | − |
| 176 | − |
| 177 | − |
| 178 | + |
| 179 | − |
| 180 | − |
| 181 | + |
| 182 | + |
| 183 | + |
| 184 | + |
| 185 | − |
| 186 | − |
| 187 | − |
| 188 | − |
| 189 | − |
| 190 | + |
| 191 | + |
| 192 | − |
| 193 | + |
| 194 | + |
| 195 | − |
| 196 | − |
| 197 | + |
| 198 | + |
| 199 | + |
| 200 | + |
| 201 | − |
| 202 | − |
| 203 | − |
| 204 | − |
| 205 | − |
| 206 | − |
| 207 | + |
| 208 | − |
| 209 | − |
| 210 | − |
| 211 | − |
| 212 | − |
| 213 | − |
| 214 | − |
| 215 | − |
| 216 | − |
| 217 | − |
| 218 | − |
| 219 | − |
| 220 | + |
| 221 | + |
| 222 | + |
| 223 | − |
| 224 | − |
| 225 | − |
| 226 | + |
| 227 | − |
| 228 | − |
| 229 | − |
| 230 | + |
| 231 | − |
| 232 | − |
| 233 | + |
| 234 | + |
| 235 | − |
| 236 | + |
| 237 | + |
| 238 | − |
| 239 | − |
| 240 | + |
| 241 | − |
| 242 | + |
| 243 | − |
| 244 | − |
| 245 | − |
| 246 | + |
| 247 | − |
| 248 | − |
| 249 | + |
| 250 | + |
| 251 | + |
| 252 | + |
| 253 | + |
| 254 | − |
| 255 | − |
| 256 | − |
| 257 | − |
| 258 | + |
| 259 | − |
| 260 | + |
| 261 | + |
| 262 | + |
| 263 | − |
| 264 | + |
| 265 | + |
| 266 | + |
| 267 | − |
| 268 | − |
| 269 | − |
| 270 | + |
| 271 | + |
| 272 | + |
| 273 | + |
| 274 | − |
| 275 | + |
| 276 | − |
| 277 | + |
| 278 | + |
| 279 | − |
| 280 | − |
| 281 | + |
| 282 | + |
| 283 | + |
| 284 | − |
| 285 | − |
| 286 | − |
| 287 | − |
| 288 | − |
| 289 | − |
| 290 | − |
| 291 | + |
| 292 | − |
| 293 | − |
| 294 | − |
| 295 | − |
| 296 | − |
| 297 | − |
| 298 | − |
| 299 | − |
| 300 | + |
| 301 | + |
| 302 | + |
| 303 | + |
| 304 | + |
| 305 | + |
| 306 | + |
| 307 | + |
| 308 | + |
| 309 | − |
| 310 | − |
| 311 | − |
| 312 | + |
| 313 | + |
| 314 | + |
| 315 | + |
| 316 | + |
| 317 | − |
| 318 | − |
| 319 | + |
| 320 | + |
| 321 | − |
| 322 | + |
| 323 | − |
| 324 | − |
| 325 | − |
| 326 | − |
| 327 | − |
| 328 | − |
| 329 | − |
| 330 | − |
| 331 | + |
| 332 | − |
| 333 | − |
| 334 | + |
| 335 | + |
| 336 | + |
| 337 | + |
| 338 | − |
| 339 | − |
| 340 | + |
| 341 | − |
| 342 | + |
| 343 | + |
| 344 | − |
| 345 | + |
| 346 | − |
| 347 | − |
| 348 | − |
| 349 | − |
| 350 | + |
| 351 | − |
| 352 | − |
| 353 | + |
| 354 | + |
| 355 | + |
| 356 | + |
| 357 | + |
| 358 | − |
| 359 | − |
| 360 | − |
| 361 | − |
| 362 | + |
| 363 | − |
| 364 | − |
| 365 | − |
| 366 | − |
| 367 | − |
| 368 | − |
| 369 | + |
| 370 | − |
| 371 | + |
| 372 | − |
| 373 | − |
| 374 | − |
| 375 | − |
| 376 | − |
| 377 | − |
| 378 | − |
| 379 | + |
| 380 | + |
| 381 | + |
| 382 | + |
| 383 | + |
| 384 | + |
| 385 | + |
| 386 | − |
| 387 | − |
| 388 | − |
| 389 | − |
| 390 | − |
| 391 | − |
| 392 | − |
| 393 | − |
| 394 | − |
| 395 | − |
| 396 | − |
| 397 | − |
| 398 | − |
| 399 | + |
| 400 | − |
| 401 | + |
| 402 | + |
| 403 | + |
| 404 | − |
| 405 | − |
| 406 | − |
| 407 | + |
| 408 | − |
| 409 | + |
| 410 | + |
| 411 | − |
| 412 | + |
| 413 | − |
| 414 | − |
| 415 | − |
| 416 | − |
| 417 | − |
| 418 | + |
| 419 | + |
| 420 | − |
| 421 | − |
| 422 | − |
| 423 | + |
| 424 | + |
| 425 | + |
| 426 | + |
| 427 | + |
| 428 | + |
| 429 | − |
| 430 | − |
| 431 | + |
| 432 | + |
| 433 | + |
| 434 | + |
| 435 | + |
| 436 | − |
| 437 | − |
| 438 | − |
| 439 | − |
| 440 | + |
| 441 | + |
| 442 | + |
| 443 | − |
| 444 | − |
| 445 | − |
| 446 | − |
| 447 | + |
| 448 | + |
| 449 | − |
| 450 | − |
| 451 | − |
| 452 | + |
| 453 | − |
| 454 | − |
| 455 | − |
| 456 | + |
| 457 | + |
| 458 | − |
| 459 | − |
| 460 | − |
| 461 | − |
| 462 | + |
| 463 | + |
| 464 | − |
| 465 | − |
| 466 | − |
| 467 | − |
| 468 | − |
| 469 | − |
| 470 | − |
| 471 | + |
| 472 | − |
| 473 | − |
| 474 | − |
| 475 | − |
| 476 | − |
| 477 | − |
| 478 | − |
| 479 | − |
| 480 | − |
| 481 | − |
| 482 | − |
| 483 | + |
| 484 | − |
| 485 | − |
| 486 | − |
| 487 | − |
| 488 | − |
| 489 | − |
| 490 | − |
| 491 | − |
| 492 | − |
| 493 | − |
| 494 | − |
| 495 | − |
| 496 | − |
| 497 | + |
| 498 | + |
| 499 | + |
| 500 | + |
| 501 | − |
| 502 | − |
| 503 | + |
| 504 | + |
| 505 | − |
| 506 | − |
| 507 | + |
| 508 | + |
| 509 | − |
| 510 | − |
| 511 | − |
| 512 | − |
| 513 | − |
| 514 | − |
| 515 | + |
| 516 | + |
| 517 | + |
| 518 | − |
| 519 | + |
| 520 | + |
| 521 | + |
| 522 | − |
| 523 | + |
| 524 | + |
| 525 | − |
| 526 | − |
| 527 | + |
| 528 | + |
| 529 | − |
| 530 | − |
| 531 | + |
| 532 | + |
| 533 | − |
| 534 | + |
| 535 | − |
| 536 | − |
| 537 | − |
| 538 | − |
| 539 | − |
| 540 | − |
| 541 | − |
| 542 | − |
| 543 | − |
| 544 | − |
| 545 | − |
| 546 | − |
| 547 | − |
| 548 | − |
| 549 | − |
| 550 | − |
| 551 | − |
| 552 | − |
| 553 | − |
| 554 | − |
| 555 | − |
| 556 | − |
| 557 | − |
| 558 | − |
| 559 | − |
| 560 | − |
| 561 | − |
| 562 | − |
| 563 | − |
| 564 | − |
| 565 | − |
| 566 | − |
| 567 | − |
| 568 | − |
| 569 | − |
| 570 | − |
| 571 | + |
| 572 | − |
| 573 | − |
| 574 | − |
| 575 | + |
| 576 | − |
| 577 | − |
| 578 | − |
| 579 | − |
| 580 | − |
| 581 | − |
| 582 | − |
| 583 | + |
| 584 | − |
| 585 | − |
| 586 | − |
| 587 | − |
| 588 | − |
| 589 | − |
| 590 | + |
| 591 | − |
| 592 | − |
| 593 | − |
| 594 | − |
| 595 | − |
| 596 | − |
| 597 | − |
| 598 | − |
| 599 | − |
| 600 | − |
| 601 | − |
| 602 | − |
| 603 | − |
| 604 | − |
| 605 | − |
| 606 | − |
| 607 | − |
| 608 | − |
| 609 | − |
| 610 | − |
| 611 | − |
| 612 | − |
| 613 | − |
| 614 | − |
| 615 | − |
| 616 | − |
| 617 | − |
| 618 | − |
| 619 | + |
| 620 | − |
| 621 | − |
| 622 | − |
| 623 | − |
| 624 | − |
| 625 | − |
| 626 | − |
| 627 | − |
| 628 | − |
| 629 | − |
| 630 | − |
| 631 | + |
| 632 | − |
| 633 | − |
| 634 | − |
| 635 | − |
| 636 | − |
| 637 | − |
| 638 | − |
| 639 | − |
| 640 | − |
| 641 | − |
| 642 | − |
| 643 | − |
| 644 | − |
| 645 | − |
| 646 | − |
| 647 | − |
| 648 | − |
| 649 | − |
| 650 | − |
| 651 | − |
| 652 | − |
| 653 | + |
| 654 | − |
| 655 | − |
| 656 | − |
| 657 | − |
| 658 | − |
| 659 | − |
| 660 | − |
| 661 | − |
| 662 | − |
| 663 | − |
| 664 | − |
| 665 | − |
| 666 | − |
| 667 | − |
| 668 | − |
| 669 | + |
| 670 | − |
| 671 | − |
| 672 | − |
| 673 | − |
| 674 | − |
| 675 | − |
| 676 | − |
| 677 | − |
| 678 | − |
| 679 | − |
| 680 | − |
| 681 | − |
| 682 | − |
| 683 | − |
| 684 | − |
| 685 | − |
| 686 | − |
| 687 | − |
| 688 | − |
| 689 | − |
| 690 | − |
| 691 | + |
| 692 | − |
| 693 | − |
| 694 | − |
| 695 | − |
| 696 | − |
| 697 | + |
| 698 | − |
| 699 | − |
| 700 | − |
| 701 | − |
| 702 | − |
| 703 | − |
| 704 | − |
| 705 | − |
| 706 | − |
| 707 | − |
| 708 | − |
| 709 | − |
| 710 | − |
| 711 | − |
| 712 | − |
| 713 | − |
| 714 | − |
| 715 | − |
| 716 | − |
| 717 | − |
| 718 | − |
| 719 | − |
| 720 | − |
| 721 | − |
| 722 | − |
| 723 | − |
| 724 | − |
| 725 | − |
| 726 | − |
| 727 | − |
| 728 | − |
| 729 | − |
| 730 | − |
| 731 | − |
| 732 | − |
| 733 | − |
| 734 | − |
| 735 | − |
| 736 | − |
| 737 | − |
| 738 | − |
| 739 | − |
| 740 | − |
| 741 | − |
| 742 | − |
| 743 | − |
| 744 | − |
| 745 | − |
| 746 | − |
| 747 | + |
| 748 | − |
| 749 | − |
| 750 | − |
| 751 | − |
| 752 | − |
| 753 | − |
| 754 | − |
| 755 | − |
| 756 | − |
| 757 | − |
| 758 | − |
| 759 | − |
| 760 | − |
| 761 | − |
| 762 | − |
| 763 | − |
| 764 | − |
| 765 | − |
| 766 | − |
| 767 | − |
| 768 | − |
| 769 | − |
| 770 | − |
| 771 | − |
| 772 | − |
| 773 | − |
| 774 | − |
| 775 | − |
| 776 | − |
| 777 | − |
| 778 | − |
| 779 | − |
| 780 | − |
| 781 | − |
| 782 | − |
| 783 | − |
| 784 | − |
| 785 | − |
| 786 | − |
| 787 | − |
| 788 | − |
| 789 | − |
| 790 | − |
| 791 | − |
| 792 | − |
| 793 | − |
| 794 | − |
| 795 | − |
| 796 | − |
| 797 | − |
| 798 | − |
| 799 | − |
| 800 | − |
| 801 | − |
| 802 | − |
| 803 | − |
| 804 | − |
| 805 | − |
| 806 | − |
| 807 | − |
| 808 | − |
| 809 | − |
| 810 | + |
| 811 | + |
| 812 | + |
| 813 | + |
| 814 | + |
| 815 | + |
| 816 | − |
| 817 | + |
| 818 | − |
| 819 | − |
| 820 | + |
| 821 | − |
| 822 | + |
| 823 | + |
| 824 | + |
| 825 | + |
| 826 | + |
| 827 | + |
| 828 | + |
| 829 | + |
| 830 | − |
| 831 | − |
| 832 | − |
| 833 | + |
| 834 | + |
| 835 | − |
| 836 | + |
| 837 | − |
| 838 | + |
| 839 | − |
| 840 | − |
| 841 | − |
| 842 | − |
| 843 | − |
| 844 | − |
| 845 | − |
| 846 | + |
| 847 | + |
| 848 | − |
| 849 | − |
| 850 | − |
| 851 | − |
| 852 | − |
| 853 | − |
| 854 | − |
| 855 | − |
| 856 | − |
| 857 | + |
| 858 | − |
| 859 | − |
| 860 | − |
| 861 | − |
| 862 | − |
| 863 | − |
| 864 | − |
| 865 | − |
| 866 | − |
| 867 | + |
| 868 | + |
| 869 | + |
| 870 | + |
| 871 | − |
| 872 | − |
| 873 | − |
| 874 | − |
| 875 | − |
| 876 | − |
| 877 | − |
| 878 | − |
| 879 | − |
| 880 | − |
| 881 | − |
| 882 | − |
| 883 | − |
| 884 | − |
| 885 | − |
| 886 | − |
| 887 | − |
| 888 | − |
| 889 | − |
| 890 | − |
| 891 | − |
| 892 | − |
| 893 | − |
| 894 | − |
| 895 | − |
| 896 | − |
| 897 | − |
| 898 | − |
| 899 | − |
| 900 | − |
| 901 | − |
| 902 | − |
| 903 | − |
| 904 | + |
| 905 | − |
| 906 | − |
| 907 | − |
| 908 | + |
| 909 | − |
| 910 | − |
| 911 | − |
| 912 | − |
| 913 | − |
| 914 | − |
| 915 | − |
| 916 | + |
| 917 | − |
| 918 | − |
| 919 | − |
| 920 | − |
| 921 | − |
| 922 | − |
| 923 | + |
| 924 | − |
| 925 | + |
| 926 | − |
| 927 | − |
| 928 | − |
| 929 | − |
| 930 | − |
| 931 | − |
| 932 | − |
| 933 | − |
| 934 | − |
| 935 | − |
| 936 | − |
| 937 | − |
| 938 | − |
| 939 | − |
| 940 | − |
| 941 | − |
| 942 | − |
| 943 | − |
| 944 | − |
| 945 | − |
| 946 | − |
| 947 | + |
| 948 | − |
| 949 | − |
| 950 | − |
| 951 | − |
| 952 | − |
| 953 | − |
| 954 | − |
| 955 | − |
| 956 | − |
| 957 | − |
| 958 | − |
| 959 | − |
| 960 | − |
| 961 | − |
| 962 | − |
| 963 | − |
| 964 | + |
| 965 | − |
| 966 | − |
| 967 | − |
| 968 | − |
| 969 | − |
| 970 | − |
| 971 | − |
| 972 | − |
| 973 | − |
| 974 | − |
| 975 | − |
| 976 | − |
| 977 | + |
| 978 | − |
| 979 | − |
| 980 | − |
| 981 | − |
| 982 | − |
| 983 | − |
| 984 | − |
| 985 | − |
| 986 | − |
| 987 | − |
| 988 | + |
| 989 | − |
| 990 | − |
| 991 | + |
| 992 | − |
| 993 | − |
| 994 | − |
| 995 | − |
| 996 | − |
| 997 | − |
| 998 | − |
| 999 | − |
| 1000 | − |
| 1001 | − |
| 1002 | − |
| 1003 | − |
| 1004 | − |
| 1005 | − |
| 1006 | − |
| 1007 | − |
| 1008 | − |
| 1009 | − |
| 1010 | − |
| 1011 | − |
| 1012 | − |
| 1013 | − |
| 1014 | − |
| 1015 | − |
| 1016 | − |
| 1017 | − |
| 1018 | − |
| 1019 | − |
| 1020 | − |
| 1021 | − |
| 1022 | − |
| 1023 | − |
| 1024 | − |
| 1025 | − |
| 1026 | − |
| 1027 | − |
| 1028 | − |
| 1029 | + |
| 1030 | − |
| 1031 | + |
| 1032 | + |
| 1033 | − |
| 1034 | − |
| 1035 | − |
| 1036 | − |
| 1037 | − |
| 1038 | − |
| 1039 | − |
| 1040 | − |
| 1041 | − |
| 1042 | − |
| 1043 | + |
| 1044 | − |
| 1045 | − |
| 1046 | − |
| 1047 | − |
| 1048 | − |
| 1049 | − |
| 1050 | − |
| 1051 | − |
| 1052 | − |
| 1053 | − |
| 1054 | − |
| 1055 | − |
| 1056 | − |
| 1057 | − |
| 1058 | − |
| 1059 | − |
| 1060 | − |
| 1061 | − |
| 1062 | − |
| 1063 | − |
| 1064 | + |
| 1065 | + |
| 1066 | − |
| 1067 | − |
| 1068 | − |
| 1069 | + |
| 1070 | − |
| 1071 | − |
| 1072 | − |
| 1073 | − |
| 1074 | + |
| 1075 | − |
| 1076 | − |
| 1077 | − |
| 1078 | − |
| 1079 | + |
| 1080 | − |
| 1081 | − |
| 1082 | − |
| 1083 | − |
| 1084 | − |
| 1085 | − |
| 1086 | + |
| 1087 | − |
| 1088 | − |
| 1089 | − |
| 1090 | − |
| 1091 | − |
| 1092 | − |
| 1093 | − |
| 1094 | − |
| 1095 | − |
| 1096 | − |
| 1097 | − |
| 1098 | − |
| 1099 | − |
| 1100 | − |
| 1101 | − |
| 1102 | − |
| 1103 | − |
| 1104 | − |
| 1105 | − |
| 1106 | − |
| 1107 | − |
| 1108 | − |
| 1109 | − |
| 1110 | − |
| 1111 | − |
| 1112 | − |
| 1113 | − |
| 1114 | − |
| 1115 | − |
| 1116 | − |
| 1117 | − |
| 1118 | − |
| 1119 | − |
| 1120 | − |
| 1121 | − |
| 1122 | + |
| 1123 | − |
| 1124 | − |
| 1125 | − |
| 1126 | − |
| 1127 | − |
| 1128 | − |
| 1129 | − |
| 1130 | − |
| 1131 | − |
| 1132 | − |
| 1133 | − |
| 1134 | − |
| 1135 | − |
| 1136 | − |
| 1137 | − |
| 1138 | − |
| 1139 | − |
| 1140 | − |
| 1141 | − |
| 1142 | − |
| 1143 | − |
| 1144 | − |
| 1145 | − |
| 1146 | − |
| 1147 | − |
| 1148 | − |
| 1149 | − |
| 1150 | − |
| 1151 | − |
| 1152 | − |
| 1153 | − |
| 1154 | − |
| 1155 | − |
| 1156 | − |
| 1157 | − |
| 1158 | − |
| 1159 | − |
| 1160 | − |
| 1161 | − |
| 1162 | − |
| 1163 | − |
| 1164 | − |
| 1165 | − |
| 1166 | − |
| 1167 | − |
| 1168 | − |
| 1169 | − |
| 1170 | + |
| 1171 | − |
| 1172 | − |
| 1173 | + |
| 1174 | − |
| 1175 | + |
| 1176 | + |
| 1177 | − |
| 1178 | − |
| 1179 | − |
| 1180 | + |
| 1181 | − |
| 1182 | + |
| 1183 | + |
| 1184 | − |
| 1185 | + |
| 1186 | − |
| 1187 | − |
| 1188 | − |
| 1189 | − |
| 1190 | − |
| 1191 | − |
| 1192 | − |
| 1193 | − |
| 1194 | − |
| 1195 | − |
| 1196 | − |
| 1197 | − |
| 1198 | − |
| 1199 | − |
| 1200 | − |
| 1201 | − |
| 1202 | − |
| 1203 | − |
| 1204 | − |
| 1205 | − |
| 1206 | − |
| 1207 | − |
| 1208 | − |
| 1209 | − |
| 1210 | − |
| 1211 | − |
| 1212 | − |
| 1213 | − |
| 1214 | − |
| 1215 | − |
| 1216 | − |
| 1217 | − |
| 1218 | − |
| 1219 | − |
| 1220 | − |
| 1221 | − |
| 1222 | − |
| 1223 | − |
| 1224 | − |
| 1225 | − |
| 1226 | − |
| 1227 | − |
| 1228 | − |
| 1229 | − |
| 1230 | − |
| 1231 | − |
| 1232 | − |
| 1233 | − |
| 1234 | − |
| 1235 | − |
| 1236 | − |
| 1237 | − |
| 1238 | − |
| 1239 | − |
| 1240 | − |
| 1241 | − |
| 1242 | − |
| 1243 | − |
| 1244 | − |
| 1245 | − |
| 1246 | − |
| 1247 | − |
| 1248 | − |
| 1249 | − |
| 1250 | − |
| 1251 | − |
| 1252 | − |
| 1253 | − |
| 1254 | − |
| 1255 | − |
| 1256 | − |
| 1257 | − |
| 1258 | − |
| 1259 | − |
| 1260 | − |
| 1261 | − |
| 1262 | − |
| 1263 | − |
| 1264 | − |
| 1265 | − |
| 1266 | − |
| 1267 | − |
| 1268 | − |
| 1269 | − |
| 1270 | − |
| 1271 | − |
| 1272 | − |
| 1273 | − |
| 1274 | − |
| 1275 | − |
| 1276 | − |
| 1277 | − |
| 1278 | − |
| 1279 | − |
| 1280 | − |
| 1281 | − |
| 1282 | − |
| 1283 | − |
| 1284 | − |
| 1285 | − |
| 1286 | − |
| 1287 | − |
| 1288 | − |
| 1289 | − |
| 1290 | − |
| 1291 | − |
| 1292 | − |
| 1293 | − |
| 1294 | − |
| 1295 | − |
| 1296 | − |
| 1297 | − |
| 1298 | − |
| 1299 | − |
| 1300 | − |
| 1301 | − |
| 1302 | − |
| 1303 | − |
| 1304 | − |
| 1305 | − |
| 1306 | + |
| 1307 | − |
| 1308 | − |
| 1309 | − |
| 1310 | − |
| 1311 | − |
| 1312 | − |
| 1313 | − |
| 1314 | − |
| 1315 | − |
| 1316 | − |
| 1317 | − |
| 1318 | − |
| 1319 | − |
| 1320 | − |
| 1321 | − |
| 1322 | − |
| 1323 | − |
| 1324 | − |
| 1325 | − |
| 1326 | − |
| 1327 | − |
| 1328 | − |
| 1329 | − |
| 1330 | − |
| 1331 | − |
| 1332 | − |
| 1333 | − |
| 1334 | − |
| 1335 | − |
| 1336 | − |
| 1337 | − |
| 1338 | − |
| 1339 | − |
| 1340 | − |
| 1341 | − |
| 1342 | − |
| 1343 | − |
| 1344 | − |
| 1345 | − |
| 1346 | − |
| 1347 | − |
| 1348 | − |
| 1349 | − |
| 1350 | − |
| 1351 | − |
| 1352 | − |
| 1353 | − |
| 1354 | − |
| 1355 | − |
| 1356 | − |
| 1357 | − |
| 1358 | − |
| 1359 | − |
| 1360 | − |
| 1361 | − |
| 1362 | − |
| 1363 | − |
| 1364 | − |
| 1365 | − |
| 1366 | − |
| 1367 | − |
| 1368 | − |
| 1369 | − |
| 1370 | − |
| 1371 | − |
| 1372 | − |
| 1373 | − |
| 1374 | − |
| 1375 | − |
| 1376 | − |
| 1377 | − |
| 1378 | − |
| 1379 | − |
| 1380 | − |
| 1381 | − |
| 1382 | − |
| 1383 | − |
| 1384 | − |
| 1385 | + |
| 1386 | − |
| 1387 | − |
| 1388 | − |
| 1389 | − |
| 1390 | − |
| 1391 | − |
| 1392 | − |
| 1393 | − |
| 1394 | − |
| 1395 | − |
| 1396 | − |
| 1397 | − |
| 1398 | − |
| 1399 | − |
| 1400 | − |
| 1401 | − |
| 1402 | − |
| 1403 | − |
| 1404 | − |
| 1405 | − |
| 1406 | − |
| 1407 | − |
| 1408 | − |
| 1409 | − |
| 1410 | − |
| 1411 | − |
| 1412 | − |
| 1413 | − |
| 1414 | − |
| 1415 | − |
| 1416 | − |
| 1417 | − |
| 1418 | − |
| 1419 | − |
| 1420 | − |
| 1421 | − |
| 1422 | − |
| 1423 | − |
| 1424 | + |
| 1425 | − |
| 1426 | − |
| 1427 | − |
| 1428 | − |
| 1429 | − |
| 1430 | − |
| 1431 | − |
| 1432 | − |
| 1433 | − |
| 1434 | − |
| 1435 | − |
| 1436 | − |
| 1437 | − |
| 1438 | − |
| 1439 | − |
| 1440 | − |
| 1441 | − |
| 1442 | − |
| 1443 | − |
| 1444 | − |
| 1445 | − |
| 1446 | − |
| 1447 | − |
| 1448 | − |
| 1449 | − |
| 1450 | − |
| 1451 | − |
| 1452 | − |
| 1453 | − |
| 1454 | − |
| 1455 | − |
| 1456 | − |
| 1457 | − |
| 1458 | − |
| 1459 | − |
| 1460 | − |
| 1461 | − |
| 1462 | − |
| 1463 | − |
| 1464 | + |
| 1465 | − |
| 1466 | − |
| 1467 | − |
| 1468 | − |
| 1469 | − |
| 1470 | − |
| 1471 | − |
| 1472 | − |
| 1473 | − |
| 1474 | − |
| 1475 | − |
| 1476 | − |
| 1477 | − |
| 1478 | − |
| 1479 | − |
| 1480 | − |
| 1481 | − |
| 1482 | − |
| 1483 | − |
| 1484 | − |
| 1485 | − |
| 1486 | − |
| 1487 | − |
| 1488 | − |
| 1489 | − |
| 1490 | − |
| 1491 | − |
| 1492 | − |
| 1493 | − |
| 1494 | − |
| 1495 | − |
| 1496 | − |
| 1497 | − |
| 1498 | − |
| 1499 | − |
| 1500 | − |
| 1501 | − |
| 1502 | − |
| 1503 | − |
| 1504 | − |
| 1505 | − |
| 1506 | − |
| 1507 | − |
| 1508 | − |
| 1509 | − |
| 1510 | − |
| 1511 | − |
| 1512 | − |
| 1513 | − |
| 1514 | + |
| 1515 | + |
| 1516 | − |
| 1517 | − |
| 1518 | − |
| 1519 | − |
| 1520 | − |
| 1521 | − |
| 1522 | − |
| 1523 | − |
| 1524 | − |
| 1525 | + |
| 1526 | − |
| 1527 | − |
| 1528 | − |
| 1529 | − |
| 1530 | − |
| 1531 | − |
| 1532 | − |
| 1533 | − |
| 1534 | − |
| 1535 | − |
| 1536 | − |
| 1537 | − |
| 1538 | − |
| 1539 | − |
| 1540 | − |
| 1541 | − |
| 1542 | − |
| 1543 | − |
| 1544 | − |
| 1545 | − |
| 1546 | − |
| 1547 | − |
| 1548 | − |
| 1549 | − |
| 1550 | − |
| 1551 | − |
| 1552 | − |
| 1553 | − |
| 1554 | + |
| 1555 | − |
| 1556 | − |
| 1557 | − |
| 1558 | − |
| 1559 | − |
| 1560 | − |
| 1561 | − |
| 1562 | − |
| 1563 | − |
| 1564 | − |
| 1565 | − |
| 1566 | − |
| 1567 | − |
| 1568 | − |
| 1569 | − |
| 1570 | − |
| 1571 | − |
| 1572 | − |
| 1573 | − |
| 1574 | − |
| 1575 | − |
| 1576 | − |
| 1577 | − |
| 1578 | − |
| 1579 | − |
| 1580 | − |
| 1581 | − |
| 1582 | − |
| 1583 | − |
| 1584 | − |
| 1585 | − |
| 1586 | − |
| 1587 | − |
| 1588 | − |
| 1589 | − |
| 1590 | − |
| 1591 | − |
| 1592 | − |
| 1593 | − |
| 1594 | − |
| 1595 | − |
| 1596 | − |
| 1597 | − |
| 1598 | − |
| 1599 | − |
| 1600 | − |
| 1601 | − |
| 1602 | − |
| 1603 | − |
| 1604 | − |
| 1605 | − |
| 1606 | − |
| 1607 | − |
| 1608 | − |
| 1609 | − |
| 1610 | − |
| 1611 | − |
| 1612 | − |
| 1613 | − |
| 1614 | − |
| 1615 | − |
| 1616 | − |
| 1617 | + |
| 1618 | − |
| 1619 | − |
| 1620 | − |
| 1621 | − |
| 1622 | − |
| 1623 | − |
| 1624 | − |
| 1625 | − |
| 1626 | − |
| 1627 | − |
| 1628 | − |
| 1629 | − |
| 1630 | − |
| 1631 | − |
| 1632 | − |
| 1633 | − |
| 1634 | − |
| 1635 | − |
| 1636 | − |
| 1637 | − |
| 1638 | − |
| 1639 | − |
| 1640 | − |
| 1641 | − |
| 1642 | − |
| 1643 | − |
| 1644 | − |
| 1645 | − |
| 1646 | − |
| 1647 | − |
| 1648 | − |
| 1649 | − |
| 1650 | − |
| 1651 | − |
| 1652 | − |
| 1653 | − |
| 1654 | − |
| 1655 | − |
| 1656 | − |
| 1657 | − |
| 1658 | + |
| 1659 | − |
| 1660 | − |
| 1661 | − |
| 1662 | − |
| 1663 | − |
| 1664 | − |
| 1665 | − |
| 1666 | − |
| 1667 | − |
| 1668 | − |
| 1669 | − |
| 1670 | − |
| 1671 | − |
| 1672 | − |
| 1673 | − |
| 1674 | − |
| 1675 | − |
| 1676 | − |
| 1677 | − |
| 1678 | − |
| 1679 | − |
| 1680 | − |
| 1681 | − |
| 1682 | − |
| 1683 | − |
| 1684 | − |
| 1685 | − |
| 1686 | − |
| 1687 | − |
| 1688 | − |
| 1689 | − |
| 1690 | − |
| 1691 | − |
| 1692 | − |
| 1693 | − |
| 1694 | − |
| 1695 | + |
| 1696 | − |
| 1697 | − |
| 1698 | − |
| 1699 | − |
| 1700 | − |
| 1701 | − |
| 1702 | + |
| 1703 | − |
| 1704 | − |
| 1705 | + |
| 1706 | + |
| 1707 | + |
| 1708 | + |
| 1709 | − |
| 1710 | − |
| 1711 | − |
| 1712 | + |
| 1713 | − |
| 1714 | + |
| 1715 | + |
| 1716 | − |
| 1717 | − |
| 1718 | + |
| 1719 | + |
| 1720 | − |
| 1721 | + |
| 1722 | − |
| 1723 | − |
| 1724 | − |
| 1725 | + |
| 1726 | − |
| 1727 | + |
| 1728 | + |
| 1729 | + |
| 1730 | + |
| 1731 | + |
| 1732 | + |
| 1733 | + |
| 1734 | − |
| 1735 | − |
| 1736 | − |
| 1737 | − |
| 1738 | + |
| 1739 | + |
| 1740 | + |
| 1741 | + |
| 1742 | + |
| 1743 | + |
| 1744 | + |
| 1745 | + |
| 1746 | + |
| 1747 | + |
| 1748 | + |
| 1749 | − |
| 1750 | + |
| 1751 | + |
| 1752 | + |
| 1753 | + |
| 1754 | + |
| 1755 | + |
| 1756 | + |
| 1757 | + |
| 1758 | + |
| 1759 | + |
| 1760 | + |
| 1761 | + |
| 1762 | − |
| 1763 | − |
| 1764 | − |
| 1765 | − |
| 1766 | + |
| 1767 | − |
| 1768 | − |
| 1769 | − |
| 1770 | − |
| 1771 | − |
| 1772 | + |
| 1773 | − |
| 1774 | − |
| 1775 | + |
| 1776 | − |
| 1777 | + |
| 1778 | + |
| 1779 | + |
| 1780 | + |
| 1781 | + |
| 1782 | − |
| 1783 | + |
| 1784 | + |
| 1785 | + |
| 1786 | + |
| 1787 | + |
| 1788 | + |
| 1789 | + |
| 1790 | + |
| 1791 | + |
| 1792 | + |
| 1793 | − |
| 1794 | − |
| 1795 | − |
| 1796 | + |
| 1797 | + |
| 1798 | + |
| 1799 | + |
| 1800 | + |
| 1801 | + |
| 1802 | + |
| 1803 | + |
| 1804 | + |
| 1805 | − |
| 1806 | − |
| 1807 | + |
| 1808 | + |
| 1809 | + |
| 1810 | − |
| 1811 | + |
| 1812 | + |
| 1813 | + |
| 1814 | + |
| 1815 | − |
| 1816 | − |
| 1817 | + |
| 1818 | + |
| 1819 | + |
| 1820 | + |
| 1821 | + |
| 1822 | + |
| 1823 | + |
| 1824 | + |
| 1825 | + |
| 1826 | − |
| 1827 | + |
| 1828 | + |
| 1829 | − |
| 1830 | + |
| 1831 | − |
| 1832 | + |
| 1833 | − |
| 1834 | − |
| 1835 | − |
| 1836 | − |
| 1837 | − |
| 1838 | − |
| 1839 | − |
| 1840 | − |
| 1841 | − |
| 1842 | − |
| 1843 | − |
| 1844 | − |
| 1845 | − |
| 1846 | − |
| 1847 | − |
| 1848 | − |
| 1849 | − |
| 1850 | + |
| 1851 | − |
| 1852 | − |
| 1853 | − |
| 1854 | − |
| 1855 | − |
| 1856 | − |
| 1857 | − |
| 1858 | − |
| 1859 | − |
| 1860 | − |
| 1861 | − |
| 1862 | + |
| 1863 | − |
| 1864 | − |
| 1865 | − |
| 1866 | − |
| 1867 | − |
| 1868 | − |
| 1869 | − |
| 1870 | − |
| 1871 | − |
| 1872 | − |
| 1873 | − |
| 1874 | − |
| 1875 | − |
| 1876 | − |
| 1877 | − |
| 1878 | − |
| 1879 | − |
| 1880 | + |
| 1881 | − |
| 1882 | − |
| 1883 | − |
| 1884 | − |
| 1885 | − |
| 1886 | − |
| 1887 | − |
| 1888 | − |
| 1889 | − |
| 1890 | − |
| 1891 | − |
| 1892 | − |
| 1893 | − |
| 1894 | − |
| 1895 | − |
| 1896 | − |
| 1897 | − |
| 1898 | − |
| 1899 | − |
| 1900 | − |
| 1901 | − |
| 1902 | − |
| 1903 | − |
| 1904 | − |
| 1905 | − |
| 1906 | − |
| 1907 | − |
| 1908 | + |
| 1909 | − |
| 1910 | − |
| 1911 | + |
| 1912 | − |
| 1913 | − |
| 1914 | − |
| 1915 | + |
| 1916 | − |
| 1917 | − |
| 1918 | − |
| 1919 | − |
| 1920 | − |
| 1921 | − |
| 1922 | − |
| 1923 | − |
| 1924 | − |
| 1925 | − |
| 1926 | − |
| 1927 | − |
| 1928 | − |
| 1929 | − |
| 1930 | − |
| 1931 | − |
| 1932 | − |
| 1933 | − |
| 1934 | − |
| 1935 | − |
| 1936 | + |
| 1937 | − |
| 1938 | − |
| 1939 | − |
| 1940 | − |
| 1941 | − |
| 1942 | − |
| 1943 | − |
| 1944 | − |
| 1945 | − |
| 1946 | + |
| 1947 | − |
| 1948 | − |
| 1949 | − |
| 1950 | − |
| 1951 | − |
| 1952 | − |
| 1953 | − |
| 1954 | − |
| 1955 | − |
| 1956 | − |
| 1957 | − |
| 1958 | − |
| 1959 | − |
| 1960 | − |
| 1961 | − |
| 1962 | − |
| 1963 | − |
| 1964 | − |
| 1965 | − |
| 1966 | − |
| 1967 | − |
| 1968 | − |
| 1969 | − |
| 1970 | − |
| 1971 | − |
| 1972 | − |
| 1973 | − |
| 1974 | + |
| 1975 | + |
| 1976 | − |
| 1977 | − |
| 1978 | − |
| 1979 | − |
| 1980 | − |
| 1981 | − |
| 1982 | − |
| 1983 | + |
| 1984 | − |
| 1985 | − |
| 1986 | − |
| 1987 | + |
| 1988 | + |
| 1989 | − |
| 1990 | − |
| 1991 | − |
| 1992 | + |
| 1993 | − |
| 1994 | + |
| 1995 | − |
| 1996 | − |
| 1997 | − |
| 1998 | − |
| 1999 | − |
| 2000 | + |
| 2001 | + |
| 2002 | + |
| 2003 | − |
| 2004 | − |
| 2005 | − |
| 2006 | − |
| 2007 | − |
| 2008 | + |
| 2009 | + |
| 2010 | + |
| 2011 | − |
| 2012 | − |
| 2013 | − |
| 2014 | − |
| 2015 | − |
| 2016 | − |
| 2017 | − |
| 2018 | − |
| 2019 | − |
| 2020 | − |
| 2021 | − |
| 2022 | − |
| 2023 | − |
| 2024 | + |
| 2025 | − |
| 2026 | + |
| 2027 | + |
| 2028 | + |
| 2029 | + |
| 2030 | − |
| 2031 | + |
| 2032 | + |
| 2033 | + |
| 2034 | − |
| 2035 | − |
| 2036 | + |
| 2037 | − |
| 2038 | + |
| 2039 | + |
| 2040 | − |
| 2041 | + |
| 2042 | + |
| 2043 | + |
| 2044 | + |
| 2045 | + |
| 2046 | − |
| 2047 | − |
| 2048 | − |
| 2049 | − |
| 2050 | − |
| 2051 | + |
| 2052 | − |
| 2053 | − |
| 2054 | − |
| 2055 | − |
| 2056 | − |
| 2057 | − |
| 2058 | − |
| 2059 | − |
| 2060 | − |
| 2061 | + |
| 2062 | − |
| 2063 | − |
| 2064 | + |
| 2065 | − |
| 2066 | − |
| 2067 | − |
| 2068 | − |
| 2069 | − |
| 2070 | − |
| 2071 | − |
| 2072 | − |
| 2073 | − |
| 2074 | − |
| 2075 | − |
| 2076 | − |
| 2077 | − |
| 2078 | − |
| 2079 | − |
| 2080 | − |
| 2081 | − |
| 2082 | + |
| 2083 | − |
| 2084 | − |
| 2085 | − |
| 2086 | − |
| 2087 | − |
| 2088 | − |
| 2089 | − |
| 2090 | − |
| 2091 | − |
| 2092 | − |
| 2093 | − |
| 2094 | − |
| 2095 | − |
| 2096 | − |
| 2097 | − |
| 2098 | − |
| 2099 | − |
| 2100 | − |
| 2101 | − |
| 2102 | + |
| 2103 | − |
| 2104 | − |
| 2105 | − |
| 2106 | − |
| 2107 | − |
| 2108 | − |
| 2109 | − |
| 2110 | − |
| 2111 | − |
| 2112 | − |
| 2113 | − |
| 2114 | − |
| 2115 | − |
| 2116 | − |
| 2117 | − |
| 2118 | − |
| 2119 | − |
| 2120 | − |
| 2121 | − |
| 2122 | − |
| 2123 | − |
| 2124 | − |
| 2125 | − |
| 2126 | − |
| 2127 | − |
| 2128 | − |
| 2129 | − |
| 2130 | − |
| 2131 | − |
| 2132 | − |
| 2133 | − |
| 2134 | − |
| 2135 | − |
| 2136 | − |
| 2137 | − |
| 2138 | − |
| 2139 | − |
| 2140 | − |
| 2141 | − |
| 2142 | − |
| 2143 | − |
| 2144 | − |
| 2145 | − |
| 2146 | − |
| 2147 | − |
| 2148 | − |
| 2149 | − |
| 2150 | + |
| 2151 | − |
| 2152 | − |
| 2153 | − |
| 2154 | − |
| 2155 | − |
| 2156 | − |
| 2157 | − |
| 2158 | + |
| 2159 | − |
| 2160 | + |
| 2161 | − |
| 2162 | − |
| 2163 | + |
| 2164 | |
| 2165 | + |
| 2166 | + |
| 2167 | + |
| 2168 | + |
| 2169 | + |
| 2170 | − |
| 2171 | − |
| 2172 | + |
| 2173 | + |
| 2174 | + |
| 2175 | + |
| 2176 | + |
| 2177 | + |
| 2178 | + |
| 2179 | + |
| 2180 | + |
| 2181 | − |
| 2182 | − |
| 2183 | − |
| 2184 | + |
| 2185 | + |
| 2186 | − |
| 2187 | − |
| 2188 | − |
| 2189 | − |
| 2190 | + |
| 2191 | + |
| 2192 | − |
| 2193 | − |
| 2194 | + |
| 2195 | − |
| 2196 | − |

### In Vivo Assay

TNF-α Production by LPS-Stimulated Mice

Male Lewis rats (180-200 g) were injected intraperitoneally with lipopolysaccharide (LPS) (50 μg/kg of E. coli strain 0111:B4, Sigma) suspended in sterile saline. Ninety minutes later, mice were sedated by CO₂:O₂ inhalation and a blood sample was obtained. Serum was separated and analyzed for TNF-α concentrations by commercial ELISA assay per the manufacturer's instructions (R&D Bioscience). Test compounds were administered orally at various times before LPS injection. The compounds were dosed either as suspensions or as solutions in various vehicles or solubilizing agents.

Compounds were dosed 0.5 to 3 hours before LPS stimulation. Rats were anaesthetized with Isofluor and injected i.v. with 0.3 mg/kg of LPS* in a volume of 0.3 ml sterile saline. Ninety minutes after the LPS injection, blood samples were collected into heparin tubes for preparation of plasma samples. Repression of TNFα production is assessed by commercial ELISA and reported below in Table 2.

EC₅₀ and percent inhibition data were obtained for the compounds provided herein. The compounds screened afforded inhibition of TNFα production as EC₅₀ values of less than 10 mg/kg in vivo. Percent inhibition data for selected compounds is shown in the Table 2 below.

In Table 2 below, the repective EC₅₀ data (+) indicates that a compound had an EC₅₀ of ≦10 mg/kg, whereas a (−) indicates that a compound had an EC₅₀ of >10 mg/kg (but were not necessarily inactive). Furthermore, % inihibition values were reported as (+) which afforded percent inihibition of >15%, and (−) to give % Inihibition ≦15%. The ND value indicates that the data was not determined for a

TABLE 2
In Vivo Activity

| | | LPS Induced TNF | LPS Induced TNF alpha |
|---|---|---|---|
| | | alpha (−1 hr) In Vivo | (−1 hr) In Vivo |
| | | (+) = ED50 ≦ 10 | (+) = >15% Inhibition |
| Example | Dose | (−) = ED50 > 10 | (−) = ≦15% Inhibition |
| 1 | 30 mg/kg | ND | + |
| 17 | 30 mg/kg | ND | + |
| 18 | 30 mg/kg | ND | + |
| 26 | 10 mg/kg | + | + |
| 27 | 10 mg/kg | − | + |
| 35 | 10 mg/kg | ND | − |
| 38 | 10 mg/kg | + | + |
| 41 | 10 mg/kg | ND | + |
| 44 | 10 mg/kg | ND | − |
| 46 | 30 mg/kg | ND | + |
| 62 | 10 mg/kg | ND | + |
| 64 | 10 mg/kg | ND | − |
| 66 | 10 mg/kg | ND | − |
| 68 | 10 mg/kg | ND | + |
| 71 | 10 mg/kg | ND | − |
| 73 | 10 mg/kg | ND | − |
| 78 | 10 mg/kg | ND | − |
| 91 | 10 mg/kg | ND | + |
| 93 | 10 mg/kg | ND | + |
| 811 | 10 mg/kg | ND | + |
| 2026 | 10 mg/kg | ND | + |
| 2027 | 10 mg/kg | ND | + |
| 2028 | 10 mg/kg | ND | − |

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of inhibition of p38 kinase comprising contacting P38 with a compound of Formula I: 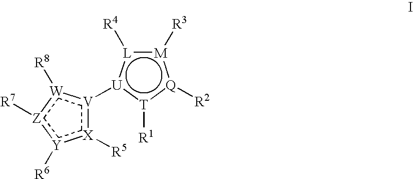
or a salt, ester, tautomer or prodrug thereof, wherein:
L, M, T, X and Y are each independently selected from the group consisting of N, C, O and S;
Q, U, V and W are each independently selected from the group consisting of N and C;
Z is selected from the group consisting of N, C(O), C, O and S;
R¹ is selected from the group consisting of alkoxy, lower alkyl, lower alkylacyl, lower alkylalkoxy, lower alkylether, amide, amino, lower aminoalkyl, halo, hydrogen, hydroxy and null, any of which may be optionally substituted;
R² is selected from the group consisting of —C(O)R⁹, —C(S)(NR¹⁰R¹¹), —C[N(OR¹²)]R¹³, —C(NR¹⁴)(NR¹⁰R¹¹) and —S(O)ₙR¹⁵;
n is 0, 1 or 2;
R³ is selected from the group consisting of alkoxy, lower alkyl, lower alkylether, amino, lower aminoalkyl, halo, haloalkyl, hydrogen, hydroxy and null, any of which may be optionally substituted;
R⁴ is selected from the group consisting of lower alkyl, halo, haloalkyl, hydrogen and null, any of which may be optionally substituted;
R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null, O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;
R⁷ is selected from the group consisting of lower alkylacyl, lower alkyl, lower alkylether, halo, hydrogen, hydroxy, lower hydroxyalkyl and null, any of which may be optionally substituted;
R⁸ is selected from the group consisting of aryl and heteroaryl, either of which may be optionally substituted;
R⁹ is selected from the group consisting of NR¹⁶R¹⁷, OR¹⁸, SR¹⁹, lower alkyl, lower alkenyl, alkynyl, amino, lower aminoalkyl, aralkyl, aryl, arylamino, arylcarbonyl, arylthio, arylsulfonyl, carbonylalkyl, carboxy, cycloalkyl, cycloalkylalkyl, cycloalkenyl, cycloalkylamino, haloalkyl, heteroaralkyl, heterocycloalkyl, heterocycloalkylalkyl, hydrogen, hydroxyalkyl, O-carbamoyl and N-carbamoyl, any of which may be optionally substituted;
R¹⁰, R¹¹, R¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted;
R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;
R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and
R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

2. The method as recited in claim 1 wherein, the compound has the Formula II: 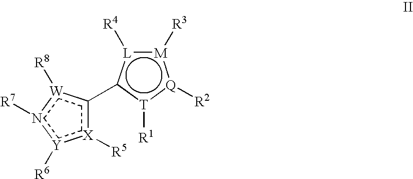
wherein:
R¹ is selected from the group consisting of lower alkyl, lower acylalkyl, lower alkoxy, amide, amino, lower aminoalkyl, lower alkylether, halo, hydrogen, hydroxy, hydroxyalkyl and null, any of which may be optionally substituted;
R² is selected from the group consisting of —C(O)R⁹, —C[N(OR¹²)]R¹³ and —S(O)ₙR¹⁵;
n is 0, 1 or 2;
R³ is selected from the group consisting of lower alkyl, lower aminoalkyl, halo, lower haloalkyl, hydrogen, hydroxy and null, any of which may be optionally substituted;
R⁴ is selected from the group consisting of lower alkyl, halo, hydrogen and null, any of which may be optionally substituted;
R⁷ is selected from the group consisting of acyl, lower alkyl, lower alkylether, hydrogen, hydroxy, hydroxyalkyl and null, any of which may be optionally substituted;
R⁹ is selected from the group consisting of NR¹⁶R¹⁷, OR¹⁸, SR¹⁹, lower alkyl, lower alkenyl, lower alkynyl, lower aminoalkyl, aralkyl, aryl, arylamino, arylcarbonyl, lower carbonylalkyl, heteroaralkyl, hydrogen and thioalkyl, any of which may be optionally substituted.

3. The method as recited in claim 2 wherein, the compound has the Formula III: 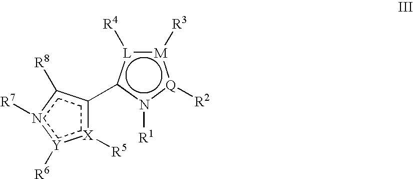
wherein:
R¹ is selected from the group consisting of lower alkoxy, lower alkyl, halo, hydrogen, hydroxy and null, any of which may be optionally substituted;
R² is selected from the group consisting of —C(O)R⁹ and —C(O)NR¹⁶R¹⁷;
n is 0, 1 or 2;
R³ is selected from the group consisting of lower alkoxy, lower alkyl, halo, hydrogen, hydroxy and null, any of which may be optionally substituted;
R⁴ is selected from the group consisting of lower alkyl, halo, haloalkyl, hydrogen and null, any of which may be optionally substituted; and
R⁷ is selected from the group consisting of lower acyl, lower alkyl, halo, hydrogen, hydroxyl and null, any of which may be optionally substituted.

4. The method as recited in claim 3, wherein R² is selected from the group consisting of —C(O)R⁹ and —C(O)NR¹⁶R¹⁷.

5. The method as recited in claim 4, wherein R⁸ is optionally substituted phenyl.

6. The method as recited in claim 5, wherein R¹⁶ is optionally substituted lower alkyl, heteroarylalkyl, cycloalkyl, cycloalkylalkyl, alkynyl or hydrogen.

7. The method as recited in claim 6, wherein R⁹ is OR¹⁸.

8. The method as recited in claim 7, wherein R¹⁸ is optionally substituted lower alkyl or hydrogen.

9. The method as recited in claim 8, wherein L is S.

10. The method as recited in claim 8, wherein Q is N.

11. The method as recited in claim 1 selected from the group consisting of Examples 1-2196.

12. A method of treatment of a p38-mediated disease in a patient in need thereof comprising the administration of a therapeutically effective amount of a compound of Formula I: 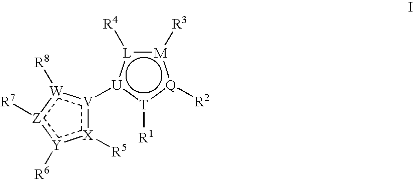
or a salt, ester, tautomer or prodrug thereof, wherein:
L, M, T, X and Y are each independently selected from the group consisting of N, C, O and S;
Q, U, V and W are each independently selected from the group consisting of N and C;
Z is selected from the group consisting of N, C(O), C, O and S;
R¹ is selected from the group consisting of alkoxy, lower alkyl, lower alkylacyl, lower alkylalkoxy, lower alkylether, amide, amino, lower aminoalkyl, halo, hydrogen, hydroxy and null, any of which may be optionally substituted;
R² is selected from the group consisting of —C(O)R⁹, —C(S)(NR¹⁰R¹¹), —C[N(OR¹²)]R¹³, —C(NR¹⁴)(NR¹⁰R¹¹) and —S(O)ₙR¹⁵;
n is 0, 1 or 2;
R³ is selected from the group consisting of alkoxy, lower alkyl, lower alkylether, amino, lower aminoalkyl, halo, haloalkyl, hydrogen, hydroxy and null, any of which may be optionally substituted;
R⁴ is selected from the group consisting of lower alkyl, halo, haloalkyl, hydrogen and null, any of which may be optionally substituted;
R⁵ and R⁶ are each independently selected from the group consisting of acyl, alkanoyl, alkoxy, alkoxyaryl, lower alkyl, alkylene, amido, amino, aminoalkyl, aryl, aralkyl, carboxy, cyano, cycloalkyl, cycloalkylalkyl, cycloalkyloxy, ester, guanidino, halo, haloalkoxy, haloalkyl, heteroaralkyl, heterocycloalkyl, heterocycloalkylalkyl, hydrogen, hydroxy, imino, iminohydroxy, nitro, null, O-carbamoyl, N-carbamoyl, S-sulfonamido, thio and ureido, any of which may be optionally substituted, or R⁵ and R⁶ may combine to form heteroaryl or heterocycloalkyl, either of which may be optionally substituted;
R⁷ is selected from the group consisting of lower alkylacyl, lower alkyl, lower alkylether, halo, hydrogen, hydroxy, lower hydroxyalkyl and null, any of which may be optionally substituted;
R⁸ is selected from the group consisting of aryl and heteroaryl, either of which may be optionally substituted;
R⁹ is selected from the group consisting of NR¹⁶R¹⁷, OR¹⁸, SR¹⁹, lower alkyl, lower alkenyl, alkynyl, amino, lower aminoalkyl, aralkyl, aryl, arylamino, arylcarbonyl, arylthio, arylsulfonyl, carbonylalkyl, carboxy, cycloalkyl, cycloalkylalkyl, cycloalkenyl, cycloalkylamino, haloalkyl, heteroaralkyl, heterocycloalkyl, heterocycloalkylalkyl, hydrogen, hydroxyalkyl, O-carbamoyl and N-carbamoyl, any of which may be optionally substituted;
R¹⁰, R¹¹, R¹⁴, R¹⁶ and R¹⁷ are each independently selected from the group consisting of acyl, lower alkenyl, alkynyl, lower alkoxy, lower alkoxyalkyl, lower alkyl, alkylthio, amino, aminoalkyl, aminocarbonyl, aralkyl, arylamino, arylcarbonyl, arylsulfonyl, cycloalkyl, cycloalkylalkyl, carboxy, cycloalkenyl, cycloalkyl, haloalkyl, hydroxyalkyl, heteroaryl, heteroaralkyl, heterocycloalkyl, heterocycloalkylalkyl and hydrogen, any of which may be optionally substituted, or either pair of R¹⁰ and R¹¹ or R¹⁶ and R¹⁷ may combine to form heterocycloalkyl, which may be optionally substituted;
R¹² and R¹³ are each independently selected from the group consisting of lower alkenyl, lower alkyl, lower alkynyl, aralkyl, aryl, cycloalkyl, cycloalkylalkyl, haloalkyl, heteroaralkyl, heterocycloalkyl, heterocycloalkylalkyl and hydrogen, any of which may be optionally substituted;
R¹⁵ is selected from the group consisting of lower alkenyl, lower alkoxy, lower alkoxyalkyl, lower alkyl, lower alkylamino, alkynyl, amino, aminocarbonylalkyl, aralkyl, arylaminocarbonyl, arylcarbonyl, arylsulfonyl, cycloalkyl, carbonylalkyl, cycloalkenyl, cycloalkyl, haloalkyl, hydroxyl, hydroxyalkyl, heteroaralkyl, heterocycloalkyl, hydrogen, thio and lower thioalkyl, any of which may be optionally substituted; and
R¹⁸ and R¹⁹ are each independently selected from the group consisting of lower alkenyl, lower alkyl, lower alkynyl, aralkyl, cycloalkyl, haloalkyl, heteroaralkyl, heterocycloalkyl and hydrogen, any of which may be optionally substituted.

13. The method as recited in claim 12 wherein said disease is selected from the group consisting of: inflammatory pain, psoriasis, acute dermatitis, arthritis, and rheumatoid arthritis.

14. A compound of Formula VII: 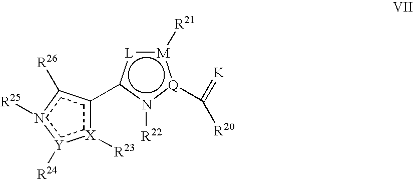
or a salt, ester, tautomer or prodrug thereof, wherein:
K is selected from the group consisting of O, S and NR²⁷;
L is selected from the group consisting of CR²⁸, NR²⁹, S and O;
Y and X are each independently selected from the group consisting of N, C, O and S;
M is selected from the group consisting of C, O and S;
Q is selected from the group consisting of C, N and S;
R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;
R²¹ is selected from the group consisting of acyl, acylalkyl, alkoxy, alkoxyalkyl, alkyl, amide, amino, aminoalkyl, hydrogen, hydroxy and null, any of which may be optionally substituted;
R²² is selected from the group consisting of alkoxy, alkyl, ether, halo, lower haloalkyl, amino, hydroxyl, lower aminoalkyl, halo, hydrogen and null, any of which may be optionally substituted;
R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;
R²⁵ is selected from the group consisting of acyl, alkyl, carboxyalkyl, ether, halo, hydrogen, hydroxy, hydroxyalkyl and null, any of which may be optionally substituted;
R²⁶ is selected from the group consisting of aryl and heteroaryl, either of which may be optionally substituted;
R²⁷ is selected from the group consisting of alkoxy, alkyl, halo and hydrogen, any of which may be optionally substituted;
R²⁸ is selected from the group consisting of alkyl, alkoxy, alkynyl, halo, haloalkyl and hydrogen, any of which may be optionally substituted;
R²⁹ is selected from the group consisting of alkoxy, alkyl, amino, hydrogen and hydroxy, any of which may be optionally substituted;
R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl, heterocycloalkyl and thioalkyl, any of which may be optionally substituted;
R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and
R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

15. The compound as recited in claim 14 having structural Formula VIII: 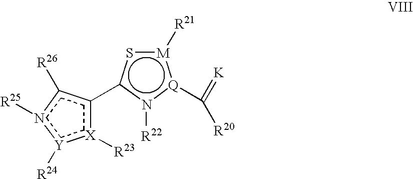
or a salt, ester, tautomer or prodrug thereof, wherein:
K is selected from the group consisting of O and NR²⁵;
Y and X are each independently selected from the group consisting of N, C, O and S;
M is selected from the group consisting of C and O;
Q is selected from the group consisting of C and N;
R²⁰ is selected from the group consisting of NR³⁰R³¹, OR³², SR³³, alkoxy, alkyl, alkenyl, alkynyl, amino, aralkyl, cycloalkyl, cycloalkenyl, haloalkyl, heteroaralkyl, heterocycloalkyl, heterocycloalkylalkyl, heterocycloalkylamino, hydrogen, O-carbamoyl, N-carbamoyl, null and thioalkyl, any of which may be optionally substituted; and
R³¹ is selected from the the group consisting of C₂₋₆ alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted.

16. The compound as recited in claim 15 having structural Formula IX: 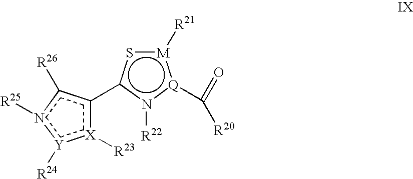
or a salt, ester, tautomer or prodrug thereof, wherein:
Y and X are each independently selected from the group consisting of N, C, O and S;
M is selected from the group consisting of C and O; and
Q is selected from the group consisting of C and N.

17. The compound as recited in claim 16 having structural Formula X: 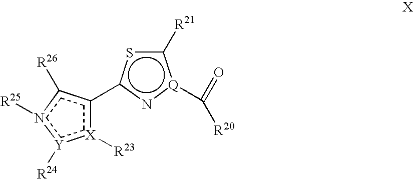
or a salt, ester, tautomer or prodrug thereof, wherein:
Y and X are each independently selected from the group consisting of N, C, O and S; and
Q is selected from the group consisting of C and N.

18. The compound as recited in claim 17 having structural Formula XI: 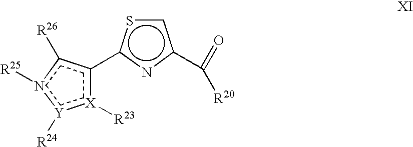
or a salt, ester, tautomer or prodrug thereof, wherein:
Y and X are each independently selected from the group consisting of N, C, O and S.

19. The compound as recited in claim 18, wherein R²⁶ is optionally substituted phenyl.

20. The compound as recited in claim 19, wherein R²³ or R²⁴ is optionally substituted alkyl, alkoxyalkyl, aminoalkyl, heterocycloalkyl, hydrogen or null.

21. The compound as recited in claim 20, wherein R²⁰ is optionally substituted amine, alkylamine, heteroarylalkyl or OR³².

22. The compound as recited in claim 14 having structural Formula XII: 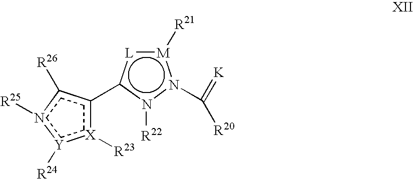
or a salt, ester, tautomer or prodrug thereof, wherein:
K is selected from the group consisting of O, S and NR²⁷;
L is selected from the group consisting of CR²⁸, NR²⁹, S and O;
Y and X are each independently selected from the group consisting of N, C, O and S;
M is selected from the group consisting of C, O and S;
Q is selected from the group consisting of C, N and S;
R²⁰ is selected from the group consisting of NR³⁰R³¹, OR³², SR³³, alkoxy, alkyl, alkenyl, alkynyl, amino, aralkyl, carbonylalkyl, cycloalkyl, cycloalkenyl, cycloalkylamino, arylamino, arylcarbonyl, arylsulfonyl, haloalkyl, heteroaralkyl, heterocycloalkyl, heterocycloalkylalkyl, heterocycloalkylamino, hydrogen, hydroxyalkyl, O-carbamoyl, N-carbamoyl, null and thioalkyl, any of which may be optionally substituted;
R²¹ is selected from the group consisting of acyl, acylalkyl, alkoxy, alkoxyalkyl, alkyl, amide, amino, aminoalkyl, hydrogen, hydroxy and null, any of which may be optionally substituted;
R²² is selected from the group consisting of alkoxy, alkyl, ether, halo, lower haloalkyl, amino, hydroxyl, lower aminoalkyl, halo, hydrogen and null, any of which may be optionally substituted;
R²³ and R²⁴ are each independently selected from the group consisting of acyl, alkanoyl, alkoxy, lower alkyl, alkylene, amido, amino, aminoalkyl, annulenyl, anthracenyl, arylalkoxy, azulenyl, benzyl, biphenyl, carboxy, cyano, cycloalkyl, cycloalkyloxy, ester, guanidino, halo, haloalkoxy, haloalkyl, heteroaryl, heterocycloalkyl, heterocycloalkylalkyl, hydrogen, hydroxy, imino, iminohydroxy, indanyl, indenyl, naphthyl, nitro, null, O-carbamoyl, N-carbamoyl, phenanthryl, tetrahydronaphthyl, thio and ureido, any of which may be optionally substituted, or R²³ and R²⁴ may combine to form heteroaryl or heterocycloalkyl, either of which may be optionally substituted;
R²⁵ is selected from the group consisting of acyl, alkyl, carboxyalkyl, ether, halo, hydrogen, hydroxy, hydroxyalkyl and null, any of which may be optionally substituted;
R²⁶ is selected from the group consisting of aryl and heteroaryl, either of which may be optionally substituted;
R²⁷ is selected from the group consisting of alkoxy, alkyl, halo and hydrogen, any of which may be optionally substituted;
R²⁸ is selected from the group consisting of alkyl, alkoxy, alkynyl, halo, haloalkyl and hydrogen, any of which may be optionally substituted;
R²⁹ is selected from the group consisting of alkoxy, alkyl, amino, hydrogen and hydroxy, any of which may be optionally substituted;
R³⁰ is selected from the group consisting of alkenyl, alkoxy, alkyl, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, hydroxyalkyl, heterocycloalkyl, and thioalkyl, any of which may be optionally substituted;
R³¹ is selected from the the group consisting of alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted; and
R³² and R³³ are each independently selected from the group consisting of alkenyl, alkyl, alkynyl, aralkyl, cycloalkyl, haloalkyl, heteroaralkyl, heterocycloalkyl and hydrogen, any of which may be optionally substituted.

23. The compound as recited in claim 22 having structural Formula XIII: 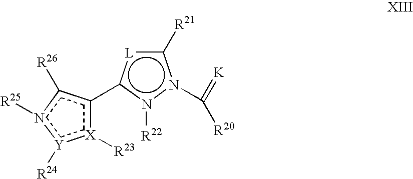
or a salt, ester, tautomer or prodrug thereof, wherein:
K is selected from the group consisting of O and NR²⁷;
L is selected from the group consisting of CR²⁸, NR²⁹, S and O;
Y and X are each selected from the group consisting of N, C, O and S;
R²⁰ is selected from the group consisting of NR³⁰R³¹, OR³², SR³³, alkoxy, alkyl, alkenyl, alkynyl, amino, aralkyl, cycloalkyl, cycloalkenyl, haloalkyl, heteroaralkyl, heterocycloalkyl, heterocycloalkylalkyl, heterocycloalkylamino, hydrogen, O-carbamoyl, N-carbamoyl, null and thioalkyl, any of which may be optionally substituted; and
R³¹ is selected from the the group consisting of C₂₋₆ alkyl, alkenyl, alkoxy, alkoxyalkyl, alkyl, alkylthio, aminoalkyl, aminocarbonylalkyl, arylaminocarbonyl, arylcarbonyl, arylsulfonyl, cycloalkyl, alkynyl, aralkyl, carbonylalkyl, cycloalkenyl, cycloalkyl, haloalkyl, heterocycloalkyl, hydroxyalkyl and hydrogen, any of which may be optionally substituted, or R³⁰ and R³¹ may combine to form heterocycloalkyl, which may be optionally substituted.

24. The compound as recited in claim 23 having structural Formula XIV: 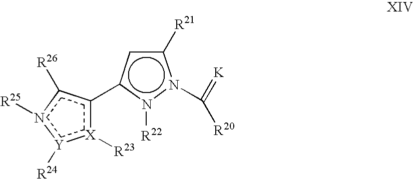
or a salt, ester, tautomer or prodrug thereof, wherein:
K is selected from the group consisting of O and NR²⁷; and
Y and X are each selected from the group consisting of N, C, O and S.

25. The compound as recited in claim 24 having structural Formula XV: 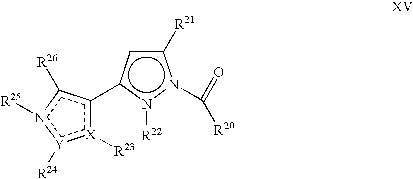
or a salt, ester, tautomer or prodrug thereof, wherein:
Y and X are each selected from the group consisting of N, C, O and S.

26. The compound as recited in claim 25, wherein R²⁶ is optionally substituted phenyl.

27. The compound as recited in claim 26, wherein R²³ or R²⁴ is optionally substituted alkyl, heterocycloalkyl, hydrogen or null.

28. The compound as recited in claim 27, wherein R²⁰ is optionally substituted alkyl, alkylamine, cycloalkylalkyl, heteroarylalkyl or arylamine.

29. The compound as recited in claim 14 selected from the group consisting of Examples 1-26, 27-33, 35-36, 38-44, 46-78, 80-97, 98-2159, 2161-2179, 2184-2185 and 2189-2194.

30. A compound as recited in claim 14 for use in the manufacture of a medicament for the prevention or treatment of a disease or condition ameliorated by the inhibition of p38 kinase.

31. A pharmaceutical composition comprising a compound as recited in claim 14 together with a pharmaceutically acceptable carrier.

32. The pharmaceutical composition as recited in claim 31, useful for the treatment or prevention of a p38-mediated disease.

33. The pharmaceutical composition as recited in claim 32, formulated for topical administration.

34. A pharmaceutical composition comprising
a) a compound as recited in claim 14, and
b) another therapeutic agent,
together with a pharmaceutically acceptable carrier.

35. The pharmaceutical composition as recited in claim 34, formulated for topical administration.

36. The pharmaceutical composition as recited in claim 35, for the treatment of inflammatory pain.
